SEQUENCE LISTING (169 sequences)

SEQ ID NO: 1 - protein, 234 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 2 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADSS

SEQ ID NO: 3 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAESS

SEQ ID NO: 4 - protein, 229 aa, synthetic construct
QVQLVQSGAEVKKPGASVKVSCKASGYTFISYTMHWVRQAPGQGLEWMGYINPRSGYTHYNQKLKDKATLTADKSASTAYMELSSLRSEDTAVYYCARSAYYDYDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 5 - protein, 235 aa, synthetic construct
MDMRVPAQLLGLLLLWLPGAKCDIQMTQSPSSLSASVGDRVTITCSASSSVSYMNWYQQKPGKAPKRLIYDTSKLASGVPSRFSGSGSGTDFTLTISSLQPEDFATYYCQQWSSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 6 - protein, 230 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 7 - protein, 213 aa, synthetic construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 8 - protein, 227 aa, synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGYGYYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 9 - protein, 214 aa, synthetic construct
DIVMTQSPSSLSASVGDRVTITCRTSRSISQYLAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 10 - protein, 231 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 11 - protein, 219 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 12 - protein, 227 aa, synthetic construct
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 13 - protein, 214 aa, synthetic construct
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 14 - protein, 226 aa, synthetic construct
EVQLVESGGGLVKPGGSLKLSCAASGLTFSSYAMSWVRQTPEKRLEWVASISSGGFTYYPDSVKGRFTISRDNARNILYLQMSSLRSEDTAMYYCARDEVRGYLDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 15 - protein, 214 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 226 aa, synthetic construct
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 17 - protein, 214 aa, synthetic construct
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 18 - protein, 227 aa, synthetic construct
QVQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 19 - protein, 219 aa, synthetic construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 20 - protein, 229 aa, synthetic construct
QVQLQQPGTELVRPGSSVKLSCKASGYTFTSYWVNWVKQRPDQGLEWIGRIDPYDSETHYNQKFTDKAISTIDTSSNTAYMQLSTLTSDASAVYYCSRSPRDSSTNLADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 21 - protein, 214 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 22 - protein, 227 aa, synthetic construct
QVQLVQSGAEDKKPGASVKVSCKASGFNIKDTYIHWVRQAPGQGLEWMGRIDPANDNTLYASKFQGRVTITADTSSNTAYMELSSLRSEDTAVYYCGRGAGAYVFDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTC

SEQ ID NO: 23 - protein, 214 aa, synthetic construct
DIVMTQSPSSLSASVGDRVTITCRTSRSISAALAWYQEKPGKAPKLLIYSGSTLQSGVPSRFSGSGSGTDFTLTISSLQPEDFATYYCQQHNENPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 24 - protein, 248 aa, synthetic construct
QVQLQQSGPGLVKPSQTLSLTCAISGDSVSSNSAAWNWIRQSPSNWIRQSPSGLEWLGRTYYRSKWYNDYAVSVKSRITINPDTSKNQFSLQLNSVTPEDTAVYYCARENIAAWTWAFDIWGQGTMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 25 - protein, 214 aa, synthetic construct
EIVMTQSPGTLSLSPGERATLSCRASQSVSSSYLAWYQQKPGLAPRLLIYDTSLRATDIPDRFSGSGSGTAFTLTISRLEPEDFAVYYCQQYGSSPTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 26 - protein, 238 aa, synthetic construct
EVQLVQSGAEVKKPGSSVKVSCKASGGTFTGYYMHWVRQAPGQGLEWMGWINPNSGGTNYAQKFQGRVTMTRDTSISTAYMELSRLRSDDTAVYYCARDLSSGYSGYFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 27 - protein, 219 aa, synthetic construct
DVVMTQSPLSLPVTPGEPASISCRSSQSLLHSNGYNYLNWYLQKPGQSPQLLIYLGSNRASGVPDRFSGSGSGTDFTLKISRVEGEDVGDYYCMQALQSPFTFGQGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 28 - protein, 238 aa, synthetic construct; identical to SEQ ID NO: 26

SEQ ID NO: 29 - protein, 219 aa, synthetic construct
DVVMTQSPLSLPVTPGEPASISCRSSQSLLHSNGYNFLDWYLQKPGQSPQLLIYLGSNRASGVPDRFSGSGSGTDFTLKISRVEADDVGVYYCMQSLQTPWTFGHGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 30 - protein, 233 aa, synthetic construct
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 31 - protein, 219 aa, synthetic construct
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 32 - protein, 232 aa, synthetic construct
EVKLVESGGGLVKPGGSLKLSCAASGFTFSSYDMSWVRQTPEKRLEWVASISGGGFLYYLDSVKGRFTISRDNARNILYLHMTSLRSEDTAMYYCARSSYGEIMDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 33 - protein, 214 aa, synthetic construct
DILLTQSPAILSVSPGERVSFSCRASQRIGTSIHWYQQRTTGSPRLLIKYASESISGIPSRFSGSGSGTDFTLSINSVESEDVADYYCQQSHGWPFTFGGGTKLEIERTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 34 - protein, 233 aa, synthetic construct
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 35 - protein, 214 aa, synthetic construct
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 36 - protein, 234 aa, synthetic construct; identical to SEQ ID NO: 1

SEQ ID NO: 37 - protein, 215 aa, synthetic construct; identical to SEQ ID NO: 3

SEQ ID NO: 38 - protein, 234 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 39 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 40 - protein, 226 aa, synthetic construct
EVQLVESGGGLVMPGGSLKLSCAASGFAFSSYDMSWVRQTPEKRLEWVAYISGGGFTYYPDTVKGRFTLSRDNAKNTLYLQMSSLKSEDTAMYYCARQGANWELVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 41 - protein, 214 aa, synthetic construct
DIVMTQSPATLSVTPGDRVFLSCRASQSISDFLHWYQQKSHESPRLLIKYASQSISGIPSRFSGSGSGSDFTLSINSVEPEDVGVYFCQNGHNFPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 227 aa, synthetic construct
QVQLQQPGAELVRPGSSVKLSCKASGYTFTRYWIHWVKQRPIQGLEWIGNIDPSDSETHYNQKFKDKATLTVDKSSGTAYMQLSSLTSEDSAVYYCATEDLYYAMEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTC

SEQ ID NO: 43 - protein, 219 aa, synthetic construct
NIMMTQSPSSLAVSAGEKVTMTCKSSQSVLYSSNQKNYLAWYQQKPGQSPKLLIYWASTRESGVPDRFTGSGSGTDFTLTISSVQPEDLAVYYCHQYLSSHTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 240 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 45 - protein, 215 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 46 - protein, 234 aa, synthetic construct
QVQLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDNYSLDYWGQGTTLTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 47 - protein, 213 aa, synthetic construct
QIVLTQSPSSLSASLGEKVTMTCRASSSVSYMNWYQQKPGTSPKRWIYDTSKVASGVPDRFSGSGSGTSYSLTISSMEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 48 - protein, 234 aa, synthetic construct
EVQLLESGGGLVQPGGSLRLSCAASGFTFSSFPMAWVRQAPGKGLEWVSTISTSGGRTYYRDSVKGRFTISRDNSKNTLYLQMNSLRAEDTAVYYCAKFRQYSGGFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 49 - protein, 216 aa, synthetic construct
DIQLTQPNSVSTSLGSTVKLSCTLSSGNIENNYVHWYQLYEGRSPTTMIYDDDKRPDGVPDRFSGSIDRSSNSAFLTIHNVAIEDEAIYFCHSYVSSFNVFGGGTKLTVLGQPKANPTVTLFPPSSEELQANKATLVCLISDFYPGAVTVAWKADGSPVKAGVETTKPSKQSNNKYAACSYLSLTPEQWKSHRSYSCQVTHEGSTVEKTVAPTESS

SEQ ID NO: 50 - protein, 237 aa, synthetic construct
EVQLVESGGGLVQPGGSLRLSCAASGYSFTGYTMNWVRQAPGKGLEWVALINPTKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 51 - protein, 214 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 52 - protein, 234 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 53 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 54 - protein, 230 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 55 - protein, 213 aa, synthetic construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 56 - protein, 226 aa, synthetic construct
EVQLVESGGGLVQPGASLKLSCVASGFTFSDYWMSWVRQTPGKPMEWIGHIKYDGSYTNYAPSLKNRFTISRDNAKTTLYLQMSNVRSEDSATYYCAREAPGAASYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 57 - protein, 219 aa, synthetic construct
DVVLTQTPVAQPVTLGDQASISCRSSQSLVHSNGNTYLEWFLQKPGQSPQLLIYKVSNRFSGVPDRFIGSGSGSDFTLKISRVEPEDWGVYYCFQGTHDPYTFGAGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 58 - protein, 229 aa, synthetic construct
EVQLQQSGAELVKPGASVKLSCRTSGFNIKDTYIHWVKQRPEQGLKWIGRIDPANGNTKYDPKFQDKATVTADTSSNTAYLQLSSLTSEDTAVYYCVTYAYDGNWYFDVWGAGTAVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 59 - protein, 214 aa, synthetic construct
DIKMTQSPSSMYVSLGERVTITCKASQDINSFLSWFQQKPGKSPKTLIYRANRLVDGVPSRFSGSGSGQDYSLTISSLEYEDMEIYYCLQYDEFPYTFGGGTKLEMKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 60 - protein, 230 aa, synthetic construct
QVQLQQPGAELVRPGTSVKLSCKASGYTFTSYWMHWIKQRPEQGLEWIGRIDPYDSETHYNEKFKDKAILSVDKSSSTAYIQLSSLTSDDSAVYYCSRRDAKYDGYALDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTC

SEQ ID NO: 61 - protein, 219 aa, synthetic construct
DIVMTQAAPSVPVTPGESVSISCRSSKTLLHSNGNTYLYWFLQRPGQSPQVLIYRMSNLASGVPNRFSGSGSETTFTLRISRVEAEDVGIYYCMQHLEYPYTFGGGTKLEIERTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 62 - protein, 236 aa, synthetic construct
EVQLVESGGGLVQPGGSLRLSCAASGYSFTGHWMNWVRQAPGKGLEWVGMIHPSDSETRYNQKFKDRFTISVDKSKNTLYLQMNSLRAEDTAVYYCARGIYFYGTTYFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 63 - protein, 214 aa, synthetic construct
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 64 - protein, 235 aa, synthetic construct
EVQLVESGGDLVQPGRSLRLSCAASGFTFSDYYMSWVRQAPGKGLEWVAAIDNDGGSISYPDTVKGRFTISRDNAKNSLYLQMNSLRVEDTALYYCARQGRLRRDYFDYWGQGTLVTVSTASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 65 - protein, 218 aa, synthetic construct
DIQMTQSPSSLSASVGDRVTITCRASESVDSYGNSFMHWYQQKPGKAPKLLIYRASNLESGVPSRFSGSGSGTDFTFTISSLQPEDIATYYCQQSNEDPLTFGQGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 66 - protein, 239 aa, synthetic construct
EVQLVESGGGLVQPGGSLRLSCATSGYTFTEYTMHWMRQAPGKGLEWVAGINPKNGGTSHNQRFMDRFTISVDKSTSTAYMQMNSLRAEDTAVYYCARWRGLNYGFDVRYFDVWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 67 - protein, 214 aa, synthetic construct
DIQMTQSPSSLSASVGDRVTITCRASQDINNYLNWYQQKPGKAPKLLIYYTSTLHSGVPSRFSGSGSGTDYTLTISSLQPEDFATYYCQQGNTLPPTFGQGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 68 - protein, 237 aa, synthetic construct
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 69 - protein, 481 aa, synthetic construct
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGESGGGGSGGGGSGGGGSQVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSGGGGSGGGGSGGGGSGGGGSDIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 70 - protein, 231 aa, synthetic construct; identical to SEQ ID NO: 10

SEQ ID NO: 71 - protein, 219 aa, synthetic construct; identical to SEQ ID NO: 11

SEQ ID NO: 72 - protein, 235 aa, synthetic construct
EVQLVESGGGLVQPGGSLRLSCAASGFTFSNYAMSWVRQAPGKRLEWVSAISDHSTNTYYPDSVKGRFTISRDNAKNTLYLQMNSLRAEDTAVYYCARKYGGDYDPFDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCHHHHHH

SEQ ID NO: 73 - protein, 214 aa, synthetic construct
DIQMTQSPSSLSASVGDRVTITCQASQDINNYIAWYQHKPGKGPKLLIHYTSTLQPGIPSRFSGSGSGRDYTLTISSLQPEDFATYYCLQYDNLLFTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 74 - protein, 136 aa, synthetic construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTLVTVSSGGCGGHHHHHH

SEQ ID NO: 75 - protein, 132 aa, synthetic construct
EVQLVESGGGLVQAGGSLRLSCAASGSTFSDYGVGWFRQAPGKGREFVADIDWNGEHTSYADSVKGRFATSRDNAKNTAYLQMNSLKPEDTAVYYCAADALPYTVRKYNYWGQGTQVTVSSGGCGGHHHHHH

SEQ ID NO: 76 - protein, 138 aa, synthetic construct
EVQLVESGGGPVQAGGSLRLSCAASGRTYRGYSMGWFRQAPGKEREFVAAIVWSGGNTYYEDSVKGRFTISRDNAKNIMYLQMTSLKPEDSATYYCAAKIRPYIFKIAGQYDYWGQGTLVTVSSAGGGSGGHHHHHHC

SEQ ID NO: 77 - protein, 133 aa, synthetic construct
EVQLVESGGGLVQPGGSLRLSCAASGDIYKSFDMGWYRQAPGKQRDLVAVIGSRGNNRGRTNYADSVKGRFTISRDGTGNTVYLLMNKLRPEDTAIYYCNTAPLVAGRPWGRGTLVTVSSGGGSGGHHHHHHC

SEQ ID NO: 78 - protein, 138 aa, synthetic construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITGGGCHHHHHHHH

SEQ ID NO: 79 - protein, 128 aa, synthetic construct
EVQLVESGGGVVQPGGSLRLSCVASGYVHKINFYGWYRQAPGKEREKVAHISIGDQTDYADSAKGRFTISRDESKNTVYLQMNSLRPEDTAAYYCRALSRIWPYDYWGQGTLVTVSSGGCGGHHHHHH

SEQ ID NO: 80 - protein, 132 aa, synthetic construct
EVQLVESGGGLVQPGGSLRLSCAASGSIFRLHTMEWYRRTPETQREWVATITSGGTTNYPDSVKGRFTISRDDTKKTVYLQMNSLKPEDTAVYYCHAVATEDAGFPPSNYWGQGTLVTVSSGGCGGHHHHHH

SEQ ID NO: 81 - protein, 135 aa, synthetic construct
EVQLVESGGGPVQAGGSLRLSCAASGRTYRGYSMGWFRQAPGREREFVAAIVWSDGNTYYEDSVKGRFTISRDNAKNTMYLQMTSLKPEDSATYYCAAKIRPYIFKIAGQYDYWGQGTLVTVSSGGCGGHHHHHH

SEQ ID NO: 82 - protein, 235 aa, synthetic construct
EVKLVESGGGLVQPGRSLRLSCAASGFNFYAYWMGWVRQAPGKGLEWIGEIKKDGTTINYTPSLKDRFTISRDNAQNTLYLQMTKLGSEDTALYYCAREERDGYFDYWGQGVMVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 83 - protein, 215 aa, synthetic construct
QFVLTQPNSVSTNLGSTVKLSCKRSTGNIGSNYVNWYQQHEGRSPTTMIYRDDKRPDGVPDRFSGSIDRSSNSALLTINNVQTEDEADYFCQSYSSGIVFGGGTKLTVLSQPKAAPSVTLFPPSSEELQANKATLVCLVSDFYPGAVTVAWKADGSPVKVGVETTKPSKQSNNKYAACSYLSLTPEQWKSHRSYSCRVTHEGSTVEKTVAPAESS

SEQ ID NO: 84 - protein, 229 aa, synthetic construct
EVQLQQSGPELVKPGASVKMSCKASGYTFTSYVIQWVKQKPGQGLEWIGSINPYNDYTKYNEKFKGKATLTSDKSSITAYMEFSLTSEDSALYCARWGDGNYWGRGTLTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 85 - protein, 211 aa, synthetic construct
DIEMTQSPAIMSASLGERVTMTCTASSSVSSSYFHWYQKPGSSPKLCIYSTSNLASGVPPRFSGSGSTSYSLTISMEAEDAATYFCHQYHRSPTFGGGTKLETKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 86 - protein, 238 aa, synthetic construct
QVQLKESGPGLVAPSQSLSITCTVSGFSLTGYGVNWVRQPPGKGLEWLGMIWGDGSTDYNSALKSRLSITKDNSKSQVFLKMNSLQTDDTARYYCARDGYSNFHYYVMDYWGQGTSVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 87 - protein, 219 aa, synthetic construct
DIVLTQSPASLAVSLGQRATISCRASESVEYYVTSLMQWYQQKPGQPPKLLISAASNVESGVPARFSGSGSGTDFSLNIHPVEEDDIAMYFCQQSRKVPWTFGGGTKLEIKRRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 88 - protein, 237 aa, synthetic construct
QVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 89 - protein, 218 aa, synthetic construct
DIVLTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 90 - protein, 234 aa, synthetic construct
QVQLQESGPGLVKPSETLSLTCAVSGFSLTSYGVHWIRQPGKGLEWLGVIWPGTNFNSALMSRLTISEDTSKNQVSLKLSSVTAADTAVYCARDRAYGNYLYAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 91 - protein, 210 aa, synthetic construct
DIQMTQSPSLSASVGDRVTITCRASESVEYVTSLMQWYQKPGKAPKLLIYAASNVDSGVPSRFSGSGTDFTLTISLQPEDIATYCQSRKVPFTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 92 - protein, 233 aa, synthetic construct
QVQLVQSGAEVKKPGASVKVSCKASGYTFTDYKIHWVRQAPGQGLEWIGYIYPYSGSSDYNQKFKSRATLTVDNSISTAYMELSRLRSDDTAVYYCARGGDAMDYWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 93 - protein, 213 aa, synthetic construct
DIQMTQSPSSLSASVGDRVTITCGASENIYGALNWYQRKPGKAPKLLIYGATNLADGVPSRFSGSGSGRDYTLTISSLQPEDFATYFCQNILGTWTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 94 - protein, 234 aa, synthetic construct
EVQLVESGGGLVQPGGSLRLSCAASGFTFNIYYMSWVRQAPGKGLELVAAINPDGGNTYYPDTVKGRFTISRDNAKNSLYLQMNSLRAEDTAVYYCARYGGPGFDSWGQGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 95 - protein, 215 aa, synthetic construct
ENVLTQSPATLSLSPGERATLSCSASSSVSYMHWYQQKPGQAPRLWIYDTSKLASGIPARFSGSGSRNDYTLTISSLEPEDFAVYYCFPGSGFPFMYTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 96 - protein, 262 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSGGGGSGGGGSGGGGSGGGGSDIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKGGGSGGCGGHHHHHH

SEQ ID NO: 97 - protein, 243 aa, synthetic construct
DVQLQESGGGLVQAGGSLRLSCAVSGYPYSSYCMGWFRQAPGKEREGVAAIDSDGRTRYADSVKGRFTISQDNAKNTLYLQMNRMKPEDTAMYYCAARFGPMGCVDLSTLSFGHWGQGTQVTVSITASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 98 - protein, 494 aa, synthetic construct
METDTLLLWVLLLWVPGSTGDYKAKEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGSGGGSGGGGSSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - DNA, 1491 bp, synthetic construct
  source 1..1491: mol_type = other DNA
atggagaccgacaccctgttgctttgggtactgttactttgggtgcccggatctaccggtgattacaaggccaaggaggtgcagctgcagcagagcggagccgagctggtgaagccaggcgcttccgtgaagatgtcttgtaaggcctccggctacacattcaccagctacaatatgcactgggtaaagcagactccggggcagggcctggagtggataggtgccatctaccctggcaacggcgacaccagctacaaccagaagtttaaggggaaggctactctaacagcggacaagtcgtcctctaccgcctacatgcaactcagctccctgacgagcgaggactccgcggactactactgtgcccgctccaactactacggctctagctattggttcttcgacgtgtggggcgctggaacgaccgtgaccgtgtcttccggtggaggttccgggggcggaagcggcggtggcggcagttcggacatcgtgctgacccagagccctgccatcctgtccgcttccccgggggagaaagttacgatgacctgccgagcgagctccagtgtcaactacatggattggtaccagaagaagcccggcagcagtcccaagccgtggatttacgctactagcaacctggcgtccggtgtcccggctcgcttctcaggttctggctcgggtactagttattcattaaccatttctcgcgtggaggctgaggacgctgccacctactactgccaacagtggtctttcaaccctcccactttcggaggcggcaccaagctcgagatcaagggcgggggtggctccgcagcagccattgaggtgatgtatcctcctccctatttggacaacgagaagtcaaatggcaccatcatccacgttaagggcaagcacctgtgcccatctcccctgttcccaggcccctctaagcccttctgggtcctggtggtggtcggcggcgtcctggcatgttactctctgctggtgaccgtcgcgttcatcatcttttgggtccggtccaagcgcagccgcctgctccactccgactacatgaatatgactcctcgtaggcccggtccaacccgcaagcactaccagccgtacgcgccgcccagagactttgctgcttaccgatccagagtgaaattttctaggtcggccgaacctcccgcatatcagcagggccagaaccagctgtacaacgaactcaacttgggacggcgcgaggaatacgatgtgctggataaacgccgtggccgcgatcccgagatgggcgggaagccacgtcgcaaaaaccctcaggagggcctttacaacgagttgcagaaggacaaaatggcggaggcctactccgagatcggaatgaagggggagcgccggcgcggcaaagggcatgacggcctctaccagggcctgtccacagccacgaaagacacctatgacgccctgcatatgcaggccctgcccccgcgctgataatga

SEQ ID NO: 100 - protein, 8 aa, synthetic construct
GSTFSDYG

SEQ ID NO: 101 - protein, 8 aa, synthetic construct
IDWNGEHT

SEQ ID NO: 102 - protein, 14 aa, synthetic construct
AADALPYTVRKYNY

SEQ ID NO: 103 - protein, 4 aa, synthetic construct
KERE

SEQ ID NO: 104 - protein, 4 aa, synthetic construct
KQRE

SEQ ID NO: 105 - protein, 4 aa, synthetic construct
GLEW

SEQ ID NO: 106 - protein, 5 aa, synthetic construct
KEREL

SEQ ID NO: 107 - protein, 5 aa, synthetic construct
KEREF

SEQ ID NO: 108 - protein, 5 aa, synthetic construct
KQREL

SEQ ID NO: 109 - protein, 5 aa, synthetic construct
KQREF

SEQ ID NO: 110 - protein, 5 aa, synthetic construct
KEREG

SEQ ID NO: 111 - protein, 5 aa, synthetic construct
KQREW

SEQ ID NO: 112 - protein, 5 aa, synthetic construct
KQREG

SEQ ID NO: 113 - protein, 4 aa, synthetic construct
TERE

SEQ ID NO: 114 - protein, 5 aa, synthetic construct
TEREL

SEQ ID NO: 115 - protein, 4 aa, synthetic construct
TQRE

SEQ ID NO: 116 - protein, 5 aa, synthetic construct
TQREL

SEQ ID NO: 117 - protein, 4 aa, synthetic construct
KECE

SEQ ID NO: 118 - protein, 5 aa, synthetic construct
KECEL

SEQ ID NO: 119 - protein, 5 aa, synthetic construct
KECER

SEQ ID NO: 120 - protein, 4 aa, synthetic construct
KQCE

SEQ ID NO: 121 - protein, 5 aa, synthetic construct
KQCEL

SEQ ID NO: 122 - protein, 4 aa, synthetic construct
RERE

SEQ ID NO: 123 - protein, 5 aa, synthetic construct
REREG

SEQ ID NO: 124 - protein, 4 aa, synthetic construct
RQRE

SEQ ID NO: 125 - protein, 5 aa, synthetic construct
RQREL

SEQ ID NO: 126 - protein, 5 aa, synthetic construct
RQREF

SEQ ID NO: 127 - protein, 5 aa, synthetic construct
RQREW

SEQ ID NO: 128 - protein, 4 aa, synthetic construct
QERE

SEQ ID NO: 129 - protein, 5 aa, synthetic construct
QEREG

SEQ ID NO: 130 - protein, 4 aa, synthetic construct
QQRE

SEQ ID NO: 131 - protein, 5 aa, synthetic construct
QQREW

SEQ ID NO: 132 - protein, 5 aa, synthetic construct
QQREL

SEQ ID NO: 133 - protein, 5 aa, synthetic construct
QQREF

SEQ ID NO: 134 - protein, 4 aa, synthetic construct
KGRE

SEQ ID NO: 135 - protein, 5 aa, synthetic construct
KGREG

SEQ ID NO: 136 - protein, 4 aa, synthetic construct
KDRE

SEQ ID NO: 137 - protein, 5 aa, synthetic construct
KDREV

SEQ ID NO: 138 - protein, 5 aa, synthetic construct
DECKL

SEQ ID NO: 139 - protein, 5 aa, synthetic construct
NVCEL

SEQ ID NO: 140 - protein, 4 aa, synthetic construct
GVEW

SEQ ID NO: 141 - protein, 4 aa, synthetic construct
EPEW

SEQ ID NO: 142 - protein, 4 aa, synthetic construct
GLER

SEQ ID NO: 143 - protein, 4 aa, synthetic construct
DQEW

SEQ ID NO: 144 - protein, 4 aa, synthetic construct
DLEW

SEQ ID NO: 145 - protein, 4 aa, synthetic construct
GIEW

SEQ ID NO: 146 - protein, 4 aa, synthetic construct
ELEW

SEQ ID NO: 147 - protein, 4 aa, synthetic construct
GPEW

SEQ ID NO: 148 - protein, 4 aa, synthetic construct
EWLP

SEQ ID NO: 149 - protein, 4 aa, synthetic construct
GPER

SEQ ID NO: 150-151 - skipped (2 entries)

SEQ ID NO: 152 - protein, 5 aa, synthetic construct
  VARIANT 1..5: note = can be present in repeats of 1, 2, 3, 4, 5, 6 or more
GGGGS

SEQ ID NO: 153 - skipped

SEQ ID NO: 154 - protein, 5 aa, synthetic construct
GGGGS

SEQ ID NO: 155 - protein, 7 aa, synthetic construct
SGGSGGS

SEQ ID NO: 156 - protein, 8 aa, synthetic construct
GGGGSGGS

SEQ ID NO: 157 - protein, 9 aa, synthetic construct
GGGGSGGGS

SEQ ID NO: 158 - protein, 10 aa, synthetic construct
GGGGSGGGGS

SEQ ID NO: 159 - protein, 15 aa, synthetic construct
GGGGSGGGGSGGGGS

SEQ ID NO: 160 - protein, 18 aa, synthetic construct
GGGGSGGGGSGGGGSGGS

SEQ ID NO: 161 - protein, 20 aa, synthetic construct
GGGGSGGGGSGGGGSGGGGS

SEQ ID NO: 162 - protein, 25 aa, synthetic construct
GGGGSGGGGSGGGGSGGGGSGGGGS

SEQ ID NO: 163 - protein, 30 aa, synthetic construct
GGGGSGGGGSGGGGSGGGGSGGGGSGGGGS

SEQ ID NO: 164 - protein, 35 aa, synthetic construct
GGGGSGGGGSGGGGSGGGGSGGGGSGGGGSGGGGS

SEQ ID NO: 165 - protein, 40 aa, synthetic construct
GGGGSGGGGSGGGGSGGGGSGGGGSGGGGSGGGGSGGGGS

SEQ ID NO: 166 - protein, 15 aa, synthetic construct
EPKSCDKTHTCPPCP

SEQ ID NO: 167 - protein, 24 aa, synthetic construct
GGGGSGGGSEPKSCDKTHTCPPCP

SEQ ID NO: 168 - protein, 12 aa, synthetic construct
EPKTPKPQPAAA

SEQ ID NO: 169 - protein, 62 aa, synthetic construct
ELKTPLGDTTHTCPRCPEPKSCDTPPPCPRCPEPKSCDTPPPCPRCPEPKSCDTPPPCPRCP

(19) United States
(12) Patent Application Publication
(10) Pub. No.: US 2026/0183418 A1
(43) Pub. Date: Jul. 2, 2026

(54) IONIZABLE CATIONIC LIPIDS AND LIPID NANOPARTICLES, AND METHODS OF SYNTHESIS AND USE THEREOF

(71) Applicant: Genzyme Corporation, Cambridge, MA (US)

(72) Inventors: Zhaohua Richard HUANG, Braintree, MA (US); Feng LIANG, Summit, NJ (US); Hong WANG, West Newton, MA (US); Sheryl WANG, Cambridge, MA (US)

(21) Appl. No.: 19/337,179

(22) Filed: Sep. 23, 2025

Related U.S. Application Data
(60) Provisional application No. 63/698,530, filed on Sep. 24, 2024.
(60) Provisional application No. 63/876,814, filed on Sep. 5, 2025.

(51) Int. Cl. A61K 47/69; A61K 47/68; C07D 317/30; C07D 407/12; C07D 409/12; C07D 409/14; C12N 15/88

(52) U.S. Cl. CPC A61K 47/6913; A61K 47/6849; C07D 317/30; C07D 407/12; C07D 409/12; C07D 409/14; C12N 15/88

(57) ABSTRACT

The invention provides ionizable cationic lipids and lipid nanoparticles for the delivery of nucleic acids to target cells, such as immune cells and hematopoietic stem cells, and methods of making and using, such lipids and targeted lipid nanoparticles.

### CROSS REFERENCE TO RELATED APPLICATIONS

This application claims priority benefit to U.S. Provisional Application No. 63/698,530, filed Sep. 24, 2024, and U.S. Provisional Application No. 63/876,814, filed Sep. 5, 2025, the disclosures of each of which are hereby incorporated herein by reference in their entireties for all purposes.

### REFERENCE TO AN ELECTRONIC SEQUENCE LISTING

The contents of the electronic sequence listing (183952035900SEQLIST.xml; Size: 168,383 bytes; and Date of Creation: Sep. 15, 2025) is herein incorporated by reference in its entirety.

### FIELD OF THE INVENTION

The invention provides ionizable cationic lipids and lipid nanoparticles for the delivery of nucleic acids to target cells, such as immune cells and hematopoietic cells, and methods of making and using, such lipids and targeted lipid nanoparticles.

### BACKGROUND

In recent years, a number of therapeutic modalities have been developed that involve the delivery of one or more nucleic acids to a subject. Treatment modalities include, for example, gene therapies where a gene of interest in the form of deoxyribose nucleic acid (DNA) is introduced into a cell, which is then expressed to produce a gene product, for example, protein, for treating a disorder caused by or associated with a deficiency or absence of the gene product. In this approach, the gene is transcribed into a messenger ribonucleic acid (mRNA), whereupon the mRNA is translated to produce the gene product. In another approach, mRNA rather than a gene of interest can be delivered to the cell. The resulting expression product can ameliorate the deficiency or absence of a particular protein in a subject (for example, a protein deficiency occurring in certain forms of cystic fibrosis or lysosomal storage disorders), or can be used to modulate a cellular function, for example, reprogramming immune cells to initiate or otherwise modulate an immune response in the subject (for example, as a therapeutic agent for treating cancer or as a prophylactic vaccine for preventing or minimizing the risk or severity of a microbial or viral infection).

However, the delivery of mRNA to a cell for translation within the cell has been challenging for a variety of factors, such as nuclease degradation of the mRNA prior to entry into the cell and then after introduction into the cell but prior to translation.

RNA may be delivered to a subject using different delivery vehicles, for example, based on cationic polymers or lipids which, together with the RNA, form nanoparticles. The nanoparticles are intended to protect the RNA from degradation, enable delivery of the RNA to the target site and facilitate cellular uptake and processing by the target cells. For delivery efficacy, in addition to the molecular composition, parameters like particle size, charge, or grafting with molecular moieties, such as polyethylene glycol (PEG) or ligands, play a role. Grafting with PEG is believed to reduce serum interactions, increase serum stability and increase time in circulation, which can be helpful for certain targeting approaches.

Compared with DNA delivery technologies used in certain gene therapies, mRNA-based gene treatment has a number of superior features, for example, ease in manipulation, rapid and transient expression, and adaptive convertibility without mutagenesis.

However, the delivery of therapeutic RNAs to cells is difficult in view of the relative instability and low cell permeability of RNAs. Thus, there exists a need to develop methods and compositions to facilitate the delivery of RNAs such as mRNA to cells.

### SUMMARY

The invention provides ionizable cationic lipids, lipid-cell targeting group conjugates, and lipid nanoparticle compositions comprising such ionizable cationic lipids and/or lipid-cell (e.g., hematopoietic stem cells or immune cells, such as T-cell, macrophage, monocytes, or dendritic cells) targeting group conjugates, medical kits comprising such lipids and/or conjugates, and methods of making and using, such lipids and conjugates.

The lipid nanoparticle compositions provided herein may further comprise a nucleic acid, such as an RNA, e.g., a messenger RNA or mRNA. The lipid nanoparticle compositions may be used for mRNA delivery to a cell (e.g., an immune cell, such as T-cell) in a subject. Messenger RNA based gene therapy requires efficient delivery of mRNA to circulating cells (e.g., immune cells, such as T-cells or NK cells) in plasma or to cells in a given tissue. The main challenges associated with efficient mRNA delivery to attain robust levels of protein expression include: (a) ability to protect the mRNA payload against prevalent serum nucleases upon administration to a subject; (b) the ability to specifically target mRNA delivery to, and thereby maximize protein expression in the target cell (e.g., T-cell, macrophage, monocytes, or dendritic cells) population; and (c) the ability to maximally deliver the mRNA payload to the cytosolic compartment of cells (e.g., T-cells) for translation into proteins within the cytoplasm.

The invention provides ionizable cationic lipids for producing lipid nanoparticle compositions that facilitate the delivery of a payload (e.g., a nucleic acid, such as a DNA or RNA, such as an mRNA) encapsulated therein to cells, for example, mammalian cells, for example, human cells, for example, immune cells. The lipids are designed to enable intracellular delivery of a nucleic acid, e.g., mRNA, to the cytosolic compartment of a target cell type and rapidly degrade into non-toxic components. These complex functionalities are achieved by the interplay between chemistry and geometry of the ionizable lipid head group, the hydrophobic “acyl-tail” groups and the linker connecting the head group and the acyl tail groups in the ionizable cationic lipids.

Also provided herein is a lipid nanoparticle (LNP) comprising a lipid blend comprising an ionizable cationic lipid and/or lipid-immune cell targeting group conjugate (e.g., a lipid-T-cell targeting group conjugate) provided herein.

In another aspect, provided herein is a method of delivering a nucleic acid to an immune cell (e.g., a T-cell), the method comprising exposing the immune cell to an LNP described herein comprising the nucleic acid under conditions that permit the nucleic acid to enter the immune cell.

In another aspect, provided herein is a method of delivering a nucleic acid to an immune cell (e.g., a T-cell) in a subject in need thereof, the method comprising administering to the subject a composition comprising an LNP described herein comprising a nucleic acid thereby to deliver the nucleic acid to the immune cell.

In another aspect, provided herein is a method of targeting the delivering of a nucleic acid (e.g., mRNA) to an immune cell (e.g., a T-cell) in a subject, the method comprising administering to the subject an LNP described herein comprising the nucleic acid so as to facilitate targeted delivery of the nucleic acid to the immune cell.

In one aspect, provided herein are lipid nanoparticles (LNPs) comprising a lipid blend for targeted delivery of a nucleic acid into an immune cell. In some embodiments, the lipid blend comprises a lipid-immune cell targeting group conjugate comprising the compound of Formula (II): [Lipid]-[optional linker]-[immune cell targeting group]. In some embodiments, the lipid blend comprises an ionizable cationic lipid. In some embodiments, the ionizable cationic lipid comprises an ionizable cationic lipid as described herein. In some embodiments, the LNP comprises a nucleic acid, wherein the nucleic acid is encapsulated in the LNP.

In some embodiments, the immune cell targeting group comprises an antibody that binds a T cell antigen. In some embodiments, the T cell antigen is CD3, CD4, CD7, or CD8, or a combination thereof (e.g., both CD3 and CD8, both CD4 and CD8, or both CD7 and CD8). In some embodiments, the immune cell targeting group comprises an antibody that binds a Natural Killer (NK) cell antigen. In some embodiments, the NK cell antigen is CD7, CD8, or CD56, or a combination thereof (e.g., both CD7 and CD8). In some embodiments, the antibody is a human or humanized antibody.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the PEG is PEG 2000.

In some embodiments, the lipid-immune cell targeting group conjugate is present in the lipid blend in a range of 0.002-0.2 mole percent. In some embodiments, the lipid blend comprises one or more of a structural lipid (e.g., a sterol), a neutral phospholipid, and a free PEG-lipid. In some embodiments, the ionizable cationic lipid is present in the lipid blend in a range of 40-60 mole percent. In some embodiments, the sterol is present in the lipid blend in a range of 30-50 mole percent. In some embodiments, the sterol is present in the lipid blend in a range of 20-70 mole percent. In some embodiments, the sterol is cholesterol.

In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), 1,2-dioleoyl-sn-glycero-3-phosphocholine (DOPC), sphingomyelin (SM). In some embodiments, the neutral phospholipid is present in the lipid blend in a range of 1-15 mole percent, such as about 5-15 mole percent.

In some embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl [methoxy (polyethylene glycol)] (PEG-ceramide), and DSPE-PEG-cysteine, or a derivative thereof. In some embodiments, the free PEG-lipid comprises a diacylphosphatidylethanolamines comprising Dipalmitoyl (C16) chain or Distearoyl (C18) chain. In some embodiments the free PEG-lipid is a mixture of two or more unique free PEG-lipids. In some embodiments, the free PEG-lipid is present in the lipid blend in a range of 1-4 mole percent, such as about 1-2 mole percent, or about 2-4 mole percent, or about 1.5 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about +5 mV to about +50 m V at pH 5, such as about +10 m V to about +30 mV at pH 5. In some embodiments, the LNP has a zeta potential of from about −10 m V to about +10 mV at pH 7.4.

In some embodiments, the nucleic acid is DNA or RNA. In some embodiments, the RNA is an mRNA, tRNA, siRNA, gRNA (guide RNA), circRNA (circular RNA), ribozymes, decoy RNA, or microRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR). In some embodiments, the CAR is TTR-023 anti-CD20 (Leu-16). In some embodiments, the CAR comprises the amino acid sequence of SEQ ID NO: 24. In some embodiments, the mRNA encoding the CAR comprises the polynucleotide sequence of 25. TTR-023 anti-CD20 (Leu-16) CAR sequence (including leader) (SEQ ID NO: 24):

(SEQ ID NO: 24)
METDTLLLWVLLLWVPGSTGDYKAKEVQLQQSGAELVKPGASVKM
SCKASGYTFTSYNMHWVKQTPGQGLEWIGAIYPGNGDTSYNQKFK
GKATLTADKSSSTAYMQLSSLTSEDSADYYCARSNYYGSSYWFFD
VWGAGTTVTVSSGGGSGGGSGGGGSSDIVLTQSPAILSASPGEKV
TMTCRASSSVNYMDWYQKKPGSSPKPWIYATSNLASGVPARFSGS
GSGTSYSLTISRVEAEDAATYYCQQWSFNPPTFGGGTKLEIKGGG
GSAAAIEVMYPPPYLDNEKSNGTIIHVKGKHLCPSPLFPGPSKPF
WVLVVVGGVLACYSLLVTVAFIIFWVRSKRSRLLHSDYMNMTPRR
PGPTRKHYQPYAPPRDFAAYRSRVKFSRSAEPPAYQQGQNQLYNE
LNLGRREEYDVLDKRRGRDPEMGGKPRRKNPQEGLYNELQKDKMA
EAYSEIGMKGERRRGKGHDGLYQGLSTATKDTYDALHMQALPPR

### Corresponding Nucleic Acid Sequence (SEQ ID NO: 25):

(SEQ ID NO: 25)
atggagaccgacaccctgttgctttgggtactgttactttgggtg
cccggatctaccggtgattacaaggccaaggaggtgcagctgcag
cagagcggagccgagctggtgaagccaggcgcttccgtgaagatg
tcttgtaaggcctccggctacacattcaccagctacaatatgcac
tgggtaaagcagactccggggcagggcctggagtggataggtgcc
atctaccctggcaacggcgacaccagctacaaccagaagittaag
gggaaggctactctaacagcggacaagtcgtcctctaccgcctac
atgcaactcagctccctgacgagcgaggactccgcggactactac
tgtgcccgctccaactactacggctctagctattggttcttcgac
gtgtggggcgctggaacgaccgtgaccgtgtcttccggtggaggt
tccgggggcggaagcggcggtggcggcagttcggacatcgtgctg
acccagagccctgccatcctgtccgcttccccgggggagaaagtt
acgatgacctgccgagcgagctccagtgtcaactacatggattgg
taccagaagaagcccggcagcagtcccaagccgtggatttacgct
actagcaacctggcgtccggtgtcccggctcgcttctcaggttct
ggctcgggtactagttattcattaaccatttctcgcgtggaggct
gaggacgctgccacctactactgccaacagtggtctttcaaccct
cccactttcggaggcggcaccaagctcgagatcaaggggggggtg
gctccgcagcagccattgaggtgatgtatcctcctccctatttgg
acaacgagaagtcaaatggcaccatcatccacgttaagggcaagc
acctgtgcccatctcccctgttcccaggcccctctaagcccttct
gggtcctggtggtggtcggcggcgtcctggcatgttactctctgc
tggtgaccgtcgcgttcatcatcttttgggtccggtccaagcgca
gccgcctgctccactccgactacatgaatatgactcctcgtaggc
ccggtccaacccgcaagcactaccagccgtacgcgccgcccagag
actttgctgcttaccgatccagagtgaaattttctaggtcggccg
aacctcccgcatatcagcagggccagaaccagctgtacaacgaac
tcaacttgggacggcgcgaggaatacgatgtgctggataaacgcc
gtggccgcgatcccgagatgggcgggaagccacgtcgcaaaaacc
ctcaggagggcctttacaacgagttgcagaaggacaaaatggcgg
aggcctactccgagatcggaatgaagggggagcgccggcgcggca
aagggcatgacggcctctaccagggcctgtccacagccacgaaag
acacctatgacgccctgcatatgcaggccctgcccccgcgctgat
aatga

In some embodiments, the immune cell targeting group comprises an antibody, and the antibody is a Fab or an immunoglobulin single variable domain, such as a Nanobody. In some embodiments, the immune cell targeting group comprises a Fab, F(ab′)₂, Fab′-SH, Fv, or scFv fragment. In some embodiments, the immune cell targeting group comprises a Fab that is engineered to knock out one or more natural interchain disulfide bonds. For example, in some embodiments, the Fab comprises a heavy chain fragment that comprises C233S substitution, numbering according to Kabat, and/or a light chain fragment that comprises C214S substitution, numbering according to Kabat. In some embodiments, the immune cell targeting group comprises a Fab that is engineered to introduce one or more buried interchain disulfide bonds. For example, in some embodiments, the Fab antibody comprises a heavy chain fragment that comprises F174C substitution, numbering according to Kabat, and/or a light chain fragment that comprises S176C substitution, numbering according to Kabat. In some embodiments, the immune cell targeting group comprises a Fab that is engineered to knock out one or more natural interchain disulfide bonds, and to introduce one or more buried interchain disulfide bonds. In some embodiments, the immune cell targeting group comprises a Fab that comprises a cysteine at the C-terminus of the heavy or light chain fragment. In some embodiments, the Fab further comprises one or more amino acids between the heavy chain fragment of the Fab and the C-terminal cysteine. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker. In some embodiments, the Fab antibody is a DS Fab (Fab with wild type (natural) interchain disulfide bond), a NoDS Fab (Fab with natural disulfide bond knocked out, such as a Fab with C233S substitution on the heavy chain, and/or C214S substitution on the light chain, numbering according to Kabat), a bDS Fab (Fab without natural disulfide bond, and with introduced non-natural interchain buried disulfide bond, such as a Fab with F174C and C233S on the heavy chain, and/or S176C and C214S substitution on the light chain, numbering according to Kabat), or a bDS Fab-ScFv (a bDS Fab linked to a ScFv through a linker, such as (G4S)x), as demonstrated in FIG. 7.

In some embodiments, the immune cell targeting group comprises an immunoglobulin single variable domain, such as a Nanobody. In some embodiments, the immunoglobulin single variable domain comprises a cysteine at the C-terminus. In some embodiments, the Nanobody further comprises a spacer comprising one or more amino acids between the Vun domain and the C-terminal cysteine. In some embodiments, the immune cell targeting group comprises two or more Van domains. In some embodiments, the two or more Vun domains are linked by an amino acid linker. In some embodiments, the immune cell targeting group comprises a first V_{HH} domain linked to an antibody CH1 domain and a second Vun domain linked to an antibody light chain constant domain, and wherein the antibody CH1 domain and the antibody light chain constant domain are linked by one or more disulfide bonds. In some embodiments, the immune cell targeting group comprises a VIH domain linked to an antibody CH1 domain, and wherein the antibody CH1 domain is linked to an antibody light chain constant domain by one or more disulfide bonds. In some embodiments, the CH1 domain comprises F174C and C233S substitutions, and/or the light chain constant domain comprises S176C and C214S substitutions, numbering according to Kabat. In some embodiments, the antibody is a ScFv, a V_{HH}(Nb), a 2×V_{HH}, a V_{HH}—CH1/empty Vk, or a V_{HH}1-CH1V_{HH}-2-Nb bDS, as demonstrated in FIG. 7.

In some embodiments, the immune cell targeting group comprises a Fab that comprises a heavy chain fragment comprising the amino acid sequence of SEQ ID NO: 1 and a light chain fragment comprising the amino acid sequence of SEQ ID NO: 2 or 3. In some embodiments, the immune cell targeting group comprises a Fab that comprises a heavy chain fragment comprising the amino acid sequence of SEQ ID NO: 6 and a light chain fragment comprising the amino acid sequence of SEQ ID NO: 7. In some embodiments, the antibody is an antibody described in the examples.

In some embodiments, the immune cell targeting group comprises a Fab that comprises:
(a) a heavy chain fragment comprising the amino acid sequence of SEQ ID NO: 1 and a light chain fragment comprising the amino acid sequence of SEQ ID NO: 2 or 3;
(b) a heavy chain fragment comprising the amino acid sequence of SEQ ID NO: 4 and a light chain fragment comprising the amino acid sequence of SEQ ID NO: 5;
(c) a heavy chain fragment comprising the amino acid sequence of SEQ ID NO: 6 and a light chain fragment comprising the amino acid sequence of SEQ ID NO: 7;
(d) a heavy chain fragment comprising the amino acid sequence of SEQ ID NO: 8 and a light chain fragment comprising the amino acid sequence of SEQ ID NO: 9;
(e) a heavy chain fragment comprising the amino acid sequence of SEQ ID NO: 10 and a light chain fragment comprising the amino acid sequence of SEQ ID NO: 11;
(f) a heavy chain fragment comprising the amino acid sequence of SEQ ID NO: 12 and a light chain fragment comprising the amino acid sequence of SEQ ID NO: 13;
(g) a heavy chain fragment comprising the amino acid sequence of SEQ ID NO: 14 and a light chain fragment comprising the amino acid sequence of SEQ ID NO: 15;
(h) a heavy chain fragment comprising the amino acid sequence of SEQ ID NO: 16 and a light chain fragment comprising the amino acid sequence of SEQ ID NO: 17;
(i) a heavy chain fragment comprising the amino acid sequence of SEQ ID NO: 18 and a light chain fragment comprising the amino acid sequence of SEQ ID NO: 19;
(j) a heavy chain fragment comprising the amino acid sequence of SEQ ID NO: 20 and a light chain fragment comprising the amino acid sequence of SEQ ID NO: 21; or
(k) a heavy chain fragment comprising the amino acid sequence of SEQ ID NO: 22 and a light chain fragment comprising the amino acid sequence of SEQ ID NO: 23.

In some embodiments, the immune cell targeting group comprises a Fab, F(ab′)₂, Fab′-SH, Fv, or scFv fragment. In some embodiments, the immune cell targeting group comprises a Fab that is engineered to knock out the natural interchain disulfide bond at the C-terminus. In some embodiments, the Fab comprises a heavy chain fragment that comprises C233S substitution, and a light chain fragment that comprises C214S substitution, numbering according to Kabat. In some embodiments, the immune cell targeting group comprises a Fab that has a non-natural interchain disulfide bond (e.g., an engineered, buried interchain disulfide bond). In some embodiments, the Fab comprises F174C substitution in the heavy chain fragment, and S176C substitution in the light chain fragment, numbering according to Kabat. In some embodiments, the immune cell targeting group comprises a Fab that comprises a cysteine at the C-terminus of the heavy or light chain fragment. In some embodiments, the Fab further comprises one or more amino acids between the heavy chain fragment of the Fab and the C-terminal cysteine.

In some embodiments, the immune cell targeting group comprises an immunoglobulin single variable domain. In some embodiments, the immunoglobulin single variable domain comprises a cysteine at the C-terminus. In some embodiments, the immunoglobulin single variable domain comprises a VHH domain and further comprises a spacer comprising one or more amino acids between the VHH domain and the C-terminal cysteine. In some embodiments, the immune cell targeting group comprises two or more VHH domains. In some embodiments, the two or more V_{HH} domains are linked by an amino acid linker. In some embodiments, the immune cell targeting group comprises a first V_{HH} domain linked to an antibody CH1 domain and a second V_{HH} domain linked to an antibody light chain constant domain. In some embodiments, the antibody CH1 domain and the antibody light chain constant domain are linked by one or more disulfide bonds. In some embodiments, the immune cell targeting group comprises a VHH domain linked to an antibody CH1 domain. In some embodiments, the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat.

In some embodiments, the immune cell targeting group comprises a Fab that comprises: (a) a heavy chain fragment comprising the amino acid sequence of SEQ ID NO: 1 and a light chain fragment comprising the amino acid sequence of SEQ ID NO: 2 or 3; or (b) a heavy chain fragment comprising the amino acid sequence of SEQ ID NO: 6 and a light chain fragment comprising the amino acid sequence of SEQ ID NO: 7. In some embodiments, the immune cell targeting group comprises a Fab that comprises: a heavy chain fragment comprising the amino acid sequence of SEQ ID NO: 1 and a light chain fragment comprising the amino acid sequence of SEQ ID NO: 2 or 3. In some embodiments, the immune cell targeting group comprises a Fab that comprises a heavy chain fragment comprising the amino acid sequence of SEQ ID NO: 6 and a light chain fragment comprising the amino acid sequence of SEQ ID NO: 7.

In another aspect, provided herein are methods of targeting the delivery of a nucleic acid to an immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the compound of the following formula (II): [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, an aspect of the disclosure relates to an LNP or a pharmaceutical composition comprising the LNP, as disclosed herein, for use in a method of targeting the delivery of a nucleic acid to an immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP). In some embodiments, the LNP is an LNP as described herein in the present disclosure.

In some aspects, provided are methods of expressing a polypeptide of interest in a targeted immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the following structure of Formula (II): [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition comprising the LNP, as disclosed herein, for use in a method of expressing a polypeptide of interest in a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some aspects, provided are methods of modulating cellular function of a target immune cell of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure of Formula (II): [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the immune cell. In some embodiments, an aspect of the disclosure relates to an LNP or a pharmaceutical composition comprising the LNP, as disclosed herein, for use in a method of modulating cellular function of a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the modulation of cell function comprises reprogramming the immune cells to initiate an immune response. In some embodiments, the modulation of cell function comprises modulating antigen specificity of the immune cell.

In some aspects, provided are methods of treating, ameliorating, or preventing a symptom of a disorder or disease in a subject in need thereof. In any of the embodiments described herein concerning a method of treating, ameliorating, and/or preventing a symptom of a disorder or disease by administration of, e.g., a LNP of the invention, it is intended that said disclosures are also interpreted as providing the, e.g., LNP for use in said methods of treating, ameliorating, and/or preventing a symptom of a disorder or disease. In some embodiments, the method comprises administering to the subject a lipid nanoparticle (LNP) for delivering a nucleic acid into an immune cell of the subject. In some embodiments, the LNP comprises an ionizable cationic lipid. In some embodiments, the LNP comprises a conjugate comprising the following structure of Formula (II): [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the nucleic acid modulates the immune response of the immune cell, therefore to treat or ameliorate the symptom. In some embodiments, an aspect of the disclosure relates to an LNP or a pharmaceutical composition comprising the LNP, as disclosed herein, for use in a method of treating, ameliorating, or preventing a symptom of a disorder or disease in a subject in need thereof. A disease or disorder may be as disclosed hereafter. In some embodiments, a method as disclosed herein can comprise contacting in vitro or ex vivo the immune cell of a subject with a lipid nanoparticle (LNP).

In some embodiments, the disorder is an immune disorder, an inflammatory disorder, or cancer. In some embodiments, the nucleic acid encodes an antigen for use in a therapeutic or prophylactic vaccine for treating or preventing an infection by a pathogen. In some embodiments, the Fab antibody comprises a heavy chain fragment that comprises F174C substitution, numbering according to Kabat, and/or a light chain fragment that comprises S176C substitution, numbering according to Kabat.

The invention provides ionizable cationic lipids and lipid nanoparticles for the delivery of nucleic acids to immune cells, and methods of making and using, such lipids and lipid nanoparticles. In some embodiments, the immune cells are macrophages, for instance M2a macrophages, M2b macrophages, and/or M2c macrophages. In some embodiments, the immune cells are B cells. In some embodiments, the immune cells are NK cells. In some embodiments, the immune cells are T cells, for example CD4+ T cells and/or CD8+ T cells. In some embodiments, the immune cells are NK cells and T cells, for example NK cells and CD4+ T cells and/or CD8+ T cells.

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In one aspect, provided are lipid nanoparticles (LNPs) for delivering a nucleic acid into NK cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody that binds CD56.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody that binds CD7 or CD8, and the free PEG-lipid is DMG-PEG or DPG-PEG.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody, and the antibody is a Fab or an immunoglobulin single variable domain. In some embodiments, the Fab is engineered to knock out the natural interchain disulfide at the C-terminus. In some embodiments, the Fab has a buried interchain disulfide. In some embodiments, the antibody is an immunoglobulin single variable (ISV) domain, and the ISV domain an Nanobody® ISV. In some embodiments, the free PEG-lipid comprise a PEG having a molecular weight of at least 2000 daltons. In some embodiments, the PEG has a molecular weight of about 3000 to 5000 daltons. In some embodiments, the Fab is an anti-CD3 antibody, and the free PEG-lipid in the LNP comprises a PEG having a molecular weight of about 2000 daltons. In some embodiments, the Fab is an anti-CD4 antibody, and the free PEG-lipid in the LNP comprises a PEG having a molecular weight of about 3000 to 3500 daltons.

In some embodiments, the free PEG-lipid comprises a diacylphosphatidylethanolamine, a dialkylphosphatidylethanolamine, a diacylglycerol, a ceramide, a dialkylglycerol, or a dialkylacetamide. In some embodiments, the alkyl chain is myristic acid, palmitic acid, oleic acid, linoleic acid, or stearic acid. In some embodiments, the free PEG-lipid is DMG-PEG. In some embodiments, free PEG-lipid is DPG-PEG.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody that binds CD3, and an antibody that binds CD11a or CD18.

In one aspect, provided are lipid nanoparticles (LNPs) for delivering a nucleic acid into immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprises an antibody that binds CD7, and an antibody that binds CD8.

In one aspect, provided are lipid nanoparticles (LNPs) for delivering a nucleic acid into two different types of immune cells of the subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid.

In some embodiments, the immune cell targeting group comprise a bispecific targeting moiety. In some embodiments, the bispecific targeting moiety binds to the two different types of immune cells. In some embodiments, the two different types of immune cells are CD4+ T cells and CD8+ T cell. In some embodiments, the bispecific targeting moiety is a bispecific antibody. In some embodiments, the bispecific antibody is a Fab-ScFv.

In one aspect, provided are lipid nanoparticles (LNPs) for delivering a nucleic acid into both CD4+ and CD8+ T cells of a subject. The LNP comprises (a) An ionizable cationic lipid, (b) A conjugate comprising the following structure: [Lipid]-[optional linker]-[immune cell targeting group]; (c) A sterol or other structural lipid; (d) A neutral phospholipid; (e) A free Polyethylene glycol (PEG) lipid, and (f) the nucleic acid. In some embodiments, the immune cell targeting group comprise a single antibody that binds to CD3 or CD7.

Further provided is a lipid nanoparticle (LNP) for delivering a nucleic acid into an immune cell of a subject, wherein the LNP comprises: (a) an ionizable cationic lipid, (b) a conjugate comprising the following structure: [Lipid]-[optional linker]-[immune cell targeting group]; (c) a sterol or other structural lipid; (d) a neutral phospholipid; (e) a free Polyethylene glycol (PEG) lipid, and (f) the nucleic acid, wherein the immune cell targeting group comprises a Fab lacking the native interchain disulfide bond. In some embodiments, the Fab is engineered to replace one or both cysteines on the native constant light chain and the native constant heavy chain that form the native interchain disulfide with a non-cysteine amino acid, therefor to remove the native interchain disulfide bond in the Fab.

Also provided is an immunoglobulin single variable domain (ISVD) that binds to human CD8. In some embodiments, the ISVD comprises three complementarity determining domains CDR1, CDR2, and CDR3, wherein
(a) the CDR1 comprises GSTFSDYG (SEQ ID NO: 100),
(b) the CDR2 comprises IDWNGEHT (SEQ ID NO: 101), and
(c) the CDR3 comprises AADALPYTVRKYNY (SEQ ID NO: 102).

In some embodiments, the ISVD is humanized.

In some embodiments, the ISVD comprises, consists of, or consists essentially of SEQ ID NO: 77.

Also provided is a polypeptide comprising GSTFSDYG (SEQ ID NO: 100), IDWNGEHT (SEQ ID NO: 101), and AADALPYTVRKYNY (SEQ ID NO: 102).

In some embodiments, the polypeptide comprises the ISVD as described herein.

In some embodiments, the polypeptide further comprises a second binding moiety, wherein the second binding moiety binds to CD8 or another different target. In some embodiments, the second binding moiety is also an ISVD.

In some embodiments, the polypeptide further comprises a detectable marker, or a therapeutic agent.

Also provided is a composition comprising the ISVD or the polypeptide as described herein.

Further provided is a pharmaceutical composition comprising the ISVD or the polypeptide as described herein, and a pharmaceutically acceptable carrier.

Further provided is a method of treating a disease or disorder related to CD8 in a subject, comprising administering the pharmaceutical composition as described herein to the subject.

In some embodiments, the disease is cancer. In some embodiments, the disorder is an immune disorder, an inflammatory disorder, or cancer.

In some embodiments, the nucleic acid encodes an antigen for use in a therapeutic or prophylactic vaccine for treating or preventing an infection by a pathogen. In some embodiments, the ionizable cationic lipid is an ionizable cationic lipid as disclosed herein, such as those in Table 1.

In some embodiments, the immune cell targeting group comprises an antibody that binds a T cell antigen. In some embodiments, the T cell antigen is CD3, CD8, or both CD3 and CD8.60. In some embodiments, the immune cell targeting group comprises an antibody that binds a Natural Killer (NK) cell antigen. In some embodiments, the NK cell antigen is CD56. In some embodiments, the antibody is a human or humanized antibody.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the PEG is PEG 2000. In some embodiments, the PEG is PEG 3400.

In some embodiments, the lipid-immune cell targeting group conjugate is present in the lipid blend in a range of 0.002-0.2 mole percent. In some embodiments, the ionizable cationic lipid is present in the lipid blend in a range of 40-60 mole percent.

In some embodiments, the sterol is cholesterol. In some embodiments, the sterol is present in the lipid blend in a range of 30-50 mole percent. In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), 1,2-dioleoyl-sn-glycero-3-phosphocholine (DOPC), sphingomyelin (SM).

In some embodiments, the neutral phospholipid is present in the lipid blend in a range of 1-15 mole percent, such as about 5 to 15 mole percent, or about 5 to 10 mole percent.

In some embodiments, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

In some embodiments, the free PEG-lipid comprises a diacylphosphatidylethanolamines comprising Dipalmitoyl (C16) chain or Distearoyl (C18) chain. In some embodiments, the free PEG-lipid is present in the lipid blend in about 0.1-4 mole percent, such as 0.5 to 2.5 mole percent, or about 1 to 2 mole percent. In some embodiments, the free PEG-lipid is present in the lipid blend in about 1.5 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about +5 mV to about +50 m V at pH 5, such as about +10 m V to about +30 mV at pH 5. In some embodiments, the LNP has a zeta potential of from about −10 m V to about +10 m V at pH 7.4.

In some embodiments, the nucleic acid is DNA or RNA. In some embodiments, the RNA is an mRNA, IRNA, siRNA, gRNA (guide RNA), circRNA (circular RNA), ribozymes, decoy RNA, or microRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

In some embodiments, the immune cell targeting group comprises an antibody, and the antibody is a Fab or an immunoglobulin single variable domain. In some embodiments, the immune cell targeting group comprises an antibody fragment selected from the group consisting of a Fab, F(ab′)₂, Fab′-SH, Fv, and scFv fragment. In some embodiments, the immune cell targeting group comprises a Fab that comprises one or more interchain disulfide bonds. In some embodiments, the Fab comprises a heavy chain fragment that comprises F174C and C233S substitutions, and a light chain fragment that comprises S176C and C214S substitutions, numbering according to Kabat. In some embodiments, the immune cell targeting group comprises a Fab that comprises a cysteine at the C-terminus of the heavy or light chain fragment.

In some embodiments, the Fab further comprises one or more amino acids between the heavy chain fragment of the Fab and the C-terminal cysteine. In some embodiments, the Fab comprises a heavy chain variable domain linked to an antibody CH1 domain and a light chain variable domain linked to an antibody light chain constant domain. In some embodiments, the CH1 domain and the light chain constant domain are linked by one or more interchain disulfide bonds. In some embodiments, the immune cell targeting group further comprises a single chain variable fragment (scFv) linked to the C-terminus of the light chain constant domain by an amino acid linker.

In some embodiments, the immune cell targeting group comprises an immunoglobulin single variable domain. In some embodiments, the immunoglobulin single variable domain comprises a cysteine at the C-terminus. In some embodiments, the immunoglobulin single variable domain comprises a VHH domain and further comprises a spacer comprising one or more amino acids between the VHH domain and the C-terminal cysteine. In some embodiments, the immune cell targeting group comprises two or more VHH domains. In some embodiments, the two or more VHH domains are linked by an amino acid linker. In some embodiments, the immune cell targeting group comprises a first VHH domain linked to an antibody CH1 domain and a second VHH domain linked to an antibody light chain constant domain. In some embodiments, the antibody CH1 domain and the antibody light chain constant domain are linked by one or more disulfide bonds. In some embodiments, the immune cell targeting group comprises a VHH domain linked to an antibody CH1 domain. In some embodiments, the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat.

In some embodiments, the immune cell targeting group comprises a Fab that comprises: a heavy chain fragment comprising the amino acid sequence of SEQ ID NO: 1 and a light chain fragment comprising the amino acid sequence of SEQ ID NO: 2 or 3.

In some embodiments, the immune cell targeting group comprises a Fab that comprises: a heavy chain fragment comprising the amino acid sequence of SEQ ID NO: 6 and a light chain fragment comprising the amino acid sequence of SEQ ID NO: 7.

In some embodiments, no more than 5% of non-immune cells are transfected by the LNP. In some embodiments, the half-life of the nucleic acid delivered by the LNP or a polypeptide encoded by the nucleic acid delivered by the LNP is at least 10% longer than the half-life of a nucleic acid delivered by a reference LNP or a polypeptide encoded by the nucleic acid delivered by a reference LNP. In some embodiments, at least 10% of immune cells are transfected by the LNP. In some embodiments, expression level of the nucleic acid delivered by the LNP is at least 10% higher than expression level of a nucleic acid delivered by a reference LNP.

In some aspects, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises free Polyethylene glycol (PEG) lipid. In some embodiments, the LNP comprises the nucleic acid. In some embodiments, the immune cell is an NK cell. In some embodiments, the immune cell targeting group comprises an antibody that binds CD56.

In some aspect, provided herein are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group comprises an antibody that binds CD7 or CD8. In some embodiments, the free PEG-lipid is DMG-PEG or DPG-PEG.

In some aspects, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises neutral phospholipid. In some embodiments, the LNP comprises a free Polyethylene glycol (PEG) lipid. In some embodiments, the LNP comprises the nucleic acid. In some embodiments, the immune cell targeting group comprises an antibody. In some embodiments, the antibody is a Fab or an immunoglobulin single variable domain.

In some embodiments, the Fab is engineered to knock out the natural interchain disulfide at the C-terminus. In some embodiments, the Fab comprises a heavy chain fragment that comprises C233S substitutions, and a light chain fragment that comprises C214S substitutions. In some embodiments, the Fab comprises a non-natural interchain disulfide. In some embodiments, the Fab comprises F174C substitution in the heavy chain fragment, and S176C substitution in the light chain fragment. In some embodiments, the antibody is an immunoglobulin single variable (ISV) domain, and the ISV domain is an Nanobody® ISV. In some embodiments, the free PEG-lipid comprises a PEG having a molecular weight of at least 2000 daltons. In some embodiments, the PEG has a molecular weight of about 3000 to 5000 daltons. In some embodiments, the antibody is a Fab. In some embodiments, the Fab binds CD3, and the free PEG-lipid in the LNP comprises a PEG having a molecular weight of about 2000 daltons. In some embodiments, the Fab is an anti-CD4 antibody, and the free PEG-lipid in the LNP comprises a PEG having a molecular weight of about 3000 to 3500 daltons.

In some embodiments, the immune cell targeting group comprises two or more VHH domains. In some embodiments, the two or more VHH domains are linked by an amino acid linker. In some embodiments, the immune cell targeting group comprises a first VHH domain linked to an antibody CH1 domain and a second VHH domain linked to an antibody light chain constant domain.

In some aspects, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the LNP binds CD3, and also binds CD11a or CD18. In some embodiments, the LNP comprises two conjugates. In some embodiments, the first conjugate comprises an antibody that binds CD3. In some embodiments, the second conjugate comprises an antibody that binds CD11a or CD18. In some embodiments, the LNP comprises one conjugate. In some embodiments, the conjugate comprises a bispecific antibody that binds both CD3 and CD11a. In some embodiments, the conjugate comprises a bispecific antibody that binds both CD3 and CD18. In some embodiments, the bispecific antibody is an immunoglobulin single variable domain or Fab-ScFv. In some aspects, provided are lipid nanoparticles (LNPs) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the LNP binds CD7 and CD8 of the immune cell.

In some embodiments, the LNP comprises two conjugates. In some embodiments, the first conjugate comprises an antibody that binds CD7, and a second conjugate that binds CD8. In some embodiments, the LNP comprises one conjugate. In some embodiments, the conjugate comprises a bispecific antibody that binds CD7 and CD8. In some embodiments, the bispecific antibody is an immunoglobulin single variable domain or Fab-ScFv.

In some aspects, provided are lipid nanoparticles (LNPs) for delivering a nucleic acid into two different types of immune cells of the subject. In some embodiments, the LNP comprises: an ionizable cationic lipid. In some embodiments, the LNP comprises a conjugate comprising the following structure: [Lipid]-[optional linker]-[immune cell targeting group]. In some embodiments, the LNP comprises sterol or other structural lipid. In some embodiments, the LNP comprises neutral phospholipid. In some embodiments, the LNP comprises a free Polyethylene glycol (PEG) lipid. In some embodiments, the LNP comprises the nucleic acid. In some embodiments, the LNP binds to a first antigen on the surface of the first type of immune cell, and also binds to a second antigen on the surface of the second type of immune cell. In some embodiments, the two different types of immune cells are CD4+ T cells and CD8+ T cell. In some embodiments, the LNP comprises two conjugates. In some embodiments, the first conjugate comprises a first antibody that binds to the first antigen of the first type of immune cell, and the second conjugate comprises a second antibody that binds to the second antigen of the second type of immune cell. In some embodiments, the LNP comprises one conjugate. In some embodiments, the conjugate comprises a bispecific antibody. In some embodiments, the bispecific antibody binds to both the first antigen on the first type of immune cell, and the second antigen on the second type of immune cells. In some embodiments, the bispecific antibody is an immunoglobulin single variable domain or a Fab-ScFv.

In some aspects, provided are lipid nanoparticles (LNPs) for delivering a nucleic acid into both CD4+ and CD8+ T cells of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group comprises a single antibody that binds to CD3 or CD7.

In some aspects, provided are lipid nanoparticles (LNPs) for delivering a nucleic acid into both T cells and NK cells of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group binds to CD7, CD8, or both CD7 and CD8.

In some aspects, provided are lipid nanoparticles (LNPs) for delivering a nucleic acid into both T cells and NK cells of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or another structural lipid. In some embodiments, the LNP comprises a neutral phospholipid. In some embodiments, the LNP comprises a free Polyethylene glycol (PEG) lipid. In some embodiments, the LNP comprises the nucleic acid. In some embodiments, the immune cell targeting group binds to (i) both CD3 and CD56; (ii) both CD8 and CDS6; or (iii) both CD7 and CD56.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), dialkylacetamide, or ceramide. In some embodiments, the lipid-immune cell targeting group conjugate is present in the lipid blend in a range of 0.002-0.2 mole percent. In some embodiments, the lipid blend further comprises one or more of a structural lipid (e.g., a sterol), a neutral phospholipid, and a free PEG-lipid.

In some embodiments, the ionizable cationic lipid is present in the lipid blend in a range of 40-60 mole percent.

In some embodiments, the sterol is present in the lipid blend in a range of 30-50 mole percent. In some embodiments, the sterol is cholesterol.

In some embodiments, the neutral phospholipid is selected from the group consisting of phosphatidylcholine, phosphatidylethanolamine, distearoyl-sn-glycero-3-phosphoethanolamine (DSPE), hydrogenated soy phosphatidylcholine (HSPC), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC). In some embodiments, the neutral phospholipid is present in the lipid blend in a range of 1-15 mole percent, such as about 5-15 mole percent.

In some embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl [methoxy (polyethylene glycol)] (PEG-ceramide), and DSPE-PEG-cysteine, or a derivative thereof. In some embodiments, the free PEG-lipid comprises a diacylphosphatidylethanolamines comprising Dipalmitoyl (C16) chain or Distearoyl (C18) chain. In some embodiments, the free PEG-lipid is present in the lipid blend in a range of 0.1 to 4 mole percent, such as about 0.5-2.5 mole percent. In some embodiment, the free PEG-lipid is about 1.5 mole percent. In some embodiments, the free PEG-lipid comprises the same or a different lipid as the lipid in the lipid-immune cell targeting group conjugate.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about −5 mV to 50 mV at pH 5, such as about +10 m V to about +30 m V at pH 5. In some embodiments, the LNP has a zeta potential of from about −10 m V to about +10 m V at pH 7.4.

In some embodiments, the nucleic acid is DNA or RNA. In some embodiments, the RNA is an mRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

In some aspects, provided are lipid nanoparticles (LNPs) for delivering a nucleic acid into an immune cell of a subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the immune cell targeting group comprises a Fab lacking the native interchain disulfide bond. In some embodiments, the Fab is engineered to replace one or both cysteines on the native constant light chain and the native constant heavy chain that form the native interchain disulfide with a non-cysteine amino acid, therefor to remove the native interchain disulfide bond in the Fab.

In some aspect, provided are methods of targeting the delivery of a nucleic acid to an immune cell of a subject. In some embodiments, the method comprises contacting the immune cell with a lipid nanoparticle (LNP) provided herein. In some embodiments, the method is for targeting NK cells. In some embodiments, the immune cell targeting group binds to CD56. In some embodiments, the method is for targeting both T cells and NK cells simultaneously. In some embodiments, the immune cell targeting group binds to CD7, CD8, or both CD7 and CD8. In some embodiments, the method is for targeting both CD4+ and CD8+ T cells simultaneously. In some embodiments, the immune cell targeting group comprises a polypeptide that binds to CD3 or CD7.

In some aspect, provided are methods of expressing a polypeptide of interest in a targeted immune cell of a subject. In some embodiments, the method comprises contacting the immune cell with a lipid nanoparticle (LNP) provided herein.

In some aspect, provided are methods of modulating cellular function of a target immune cell of a subject. In some embodiments, the methods comprise administering to the subject a lipid nanoparticle (LNP) provided herein.

In some aspect, provided are methods of treating, ameliorating, or preventing a symptom of a disorder or disease in a subject in need thereof. In some embodiments, the methods comprise administering to the subject a lipid nanoparticle (LNP) provided herein.

In some aspects, provided are immunoglobulin single variable domains (ISVDs) that bind to human CD8. In some embodiments, the ISVD comprises three complementarity determining domains CDR1, CDR2, and CDR3. In some embodiments, the CDR1 comprises GSTFSDYG (SEQ ID NO: 100). In some embodiments, the CDR2 comprises IDWNGEHT (SEQ ID NO: 101). In some embodiments, the CDR3 comprises AADALPYTVRKYNY (SEQ ID NO: 102). In some embodiments, the ISVD is humanized. In some embodiments, the ISVD comprises SEQ ID NO: 77.

In some aspects, provided are polypeptides comprising GSTFSDYG (SEQ ID NO: 100), IDWNGEHT (SEQ ID NO: 101), and AADALPYTVRKYNY (SEQ ID NO: 102). In another aspect, provided are polypeptides comprising the ISVD provided herein. In some embodiments, the polypeptide comprises a second binding moiety. In some embodiments, the second binding moiety binds to CD8 or another different target. In some embodiments, the second binding moiety is also an ISVD. In some embodiments, the polypeptide comprises a detectable marker. In some embodiments, the polypeptide comprises a therapeutic agent.

In some aspects, provided are compositions comprising the ISVD provided herein or the polypeptide provided herein.

In some aspects, provided are pharmaceutical compositions comprising the ISVD provided herein or the polypeptide provided herein, and a pharmaceutically acceptable carrier.

In some aspects, provided are methods of treating a disease or disorder related to CD8 in a subject. In some embodiments, the method comprises administering a pharmaceutical composition described herein to the subject. In some embodiments, the disease or disorder is cancer,

In some aspects, provided is a compound of Formula (I):

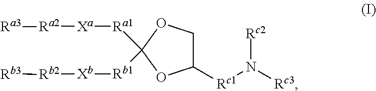

or a salt thereof, wherein: R^{a1} and R^{b1} are each independently C₁₋₁₂ alkylene; X^{a} and X^{b} are each independently —C(O)O—* or —OC(O)—*, wherein * indicates the point of attachment to R^{a1} or R^{b1}; R^{a2} and R^{b2} are each independently a bond or C₁₋₃ alkylene; R^{a3} is

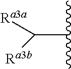

### and R^{b3} is

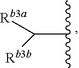

wherein R^{a3a}, R^{a3b}, R^{b3a}, and R^{b3b} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3; R^{c1} is C₁₋₆ alkylene; R^{c2} is H or C16 alkyl; and R^{c3} is C₁₋₆ alkyl

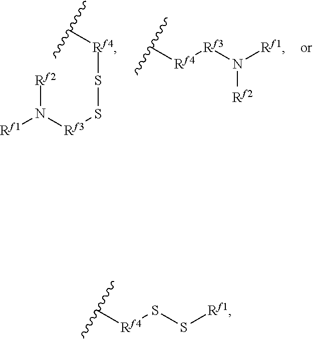

wherein: R^{f1} is H, C₁₋₆ alkyl, or

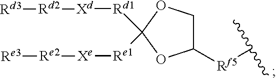

R^{f2} is H, C₁₋₆ alkyl, or —C(O)O—C₂₋₆ alkenyl; R^{f3}, R^{f4}, and R^{f5} are each independently C₁₋₆ alkylene; and R^{d1} and R^{e1} are each independently C₁₋₁₂ alkylone; X^{d} and X^{e} are each independently —C(O)O—* or —OC(O)—*, wherein * indicates the point of attachment to R^{d1} or Rel; R^{d2} and R^{e2} are each independently a bond or C₁₋₃ alkylene; and R^{d3} is

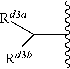

### and R^{c3} is

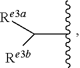

wherein R^{d3a}, R^{d3b}, R^{c3a}, and R^{c3b} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3; with the proviso that when R^{c1} is —CH₂— and R^{c2} and R^{c3} are each methyl, then none of R^{a3a}, R^{a3b}, R^{b3a}, and R^{b3b} is H; when R^{c1} is —(CH₂)₂— and R^{c2} and R^{c3} are each methyl, then R^{b3a} is not H and R^{b3b} is ethyl; when R^{c1} is —(CH₂)₂— and R^{c2}, R^{c3}, or both R^{c2} and R^{c3} are not methyl, then none of R^{a3a}, R^{a3b}, R^{b3a}, and R^{b3b} is H; when R^{c1} is —(CH₂)₃—, R^{c2} and R^{c3} are each methyl, and one of R^{a3a}, R^{a3b}, R^{b3a}, and R^{b3b} is H, then at least one of R^{a3a}, R^{a3b}, R^{b3a}, and R^{b3b} that is not His substituted with a heterocylyl; and when R^{c1} is —(CH₂)₄—, R^{c2} and R^{c3} are each methyl, then none of R^{a3a}, R^{a3b}, R^{b3a}, and R^{b3b} is H.

In some embodiments, R^{a1} and R^{b1} are each independently a linear C₁₋₁₂ alkylene. In some embodiments, R^{a1} and R^{b1} are each independently C₅₋₁₀ alkylene. In some embodiments, R^{a1} and R^{b1} are each —(CH₂)₇—.

In some embodiments, X^{a} and X^{b} are each —C(O)O—*. In some embodiments, X^{a} and X are each —OC(O)—*.

In some embodiments, R^{a2} and R^{b2} are each a bond. In some embodiments, R^{a2} and R^{b2} are each —CH₂—.

In some embodiments, no more than one of R^{a3a}, R^{a3b}, R^{b3a}, and R^{b3b} is H. In some embodiments, R^{a3a}, R^{a3b}, R^{b3a}, and R^{b3b} are each independently a linear C₁₋₁₂ alkyl. In some embodiments, R^{a3a}, R^{a3b}, R^{53a}, and R^{b3b} are each independently C₂₋₁₀ alkyl. In some embodiments, R^{a3a}, R^{a3b}, R^{b3a}, and R^{53b} are each independently H or optionally substituted with a heterocyclyl comprising a disulfide bond. In some embodiments, R^{a3a}, R^{a3b}, R^{b3a}, and R^{b3b} are each independently H or optionally substituted with 1,2-dithiolanyl. In some embodiments, none of R^{a3a}, R^{a3b}, R^{b3a}, and R^{b3b} is H. In some embodiments, at least one of R^{a3a}, R^{a3b}, R^{b3a}, and R^{b3b} is substituted with a heterocyclyl. In some embodiments, at least one of R^{a3a}, R^{a3b}, R^{b3a}, and R^{b3b} is C₁₋₁₂ alkyl substituted with a 5- to 10-membered heterocyclyl comprising a disulfide bond or —(C₁₋₁₀ alkylene)-Sₙ—(C₁₋₁₀ alkyl). In some embodiments, the heterocyclyl comprising a disulfide bond is 1,2-dithiolanyl.

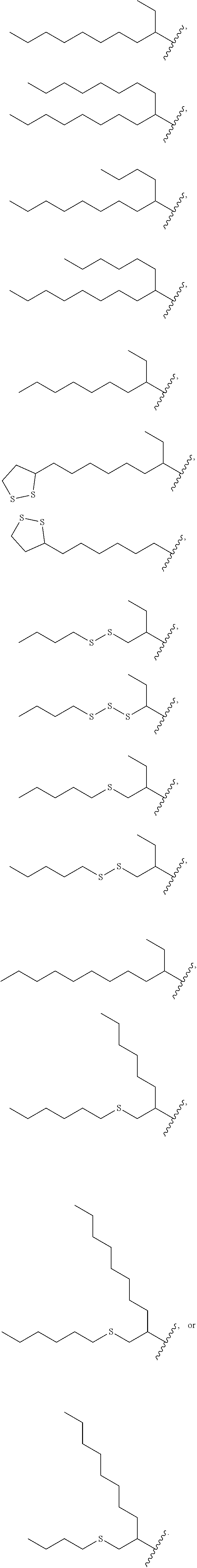

In some embodiments, R^{a3} and R^{b3} are each independently

In some embodiments, R^{a3} and R^{b3} are the same.

In some embodiments, R^{c1} is —(CH₂)₂—. In some embodiments, R^{c1} is —(CH₂)₃—. In some embodiments, R^{c1} is —(CH₂)₄—.

In some embodiments, R^{c2} is methyl. In some embodiments, R^{c2} is ethyl.

In some embodiments, R^{c3} is C₁₋₆ alkyl. In some embodiments, R^{c3} is methyl. In some embodiments, R^{c3} is ethyl.

In some embodiments, when R^{c1} is —(CH₂)₂— and R^{c2} is methyl, then R^{c3} is not methyl.

In some embodiments, R^{c3} is

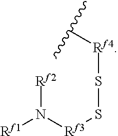

In some embodiments, R^{c3} is

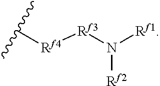

In some embodiments, R^{c3} is

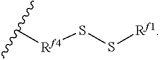

In some embodiments, R^{f1} is H. In some embodiments, R^{f1} is methyl. In some embodiments, R^{f1} is C₁₋₆ alkyl. In some embodiments, R¹¹ is n-butyl.

In some embodiments, R^{f1} is

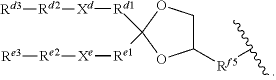

In some embodiments, R^{f2} is H. In some embodiments, R¹² is methyl. In some embodiments, R^{f2} is ethyl. In some embodiments, R¹² is —C(O)O—C₂₋₆ alkenyl. In some embodiments, R¹² is —C(O)O—CH₂CH═CH₂. In some embodiments, R¹² is C₁₋₆ alkyl.

In some embodiments, R^{f3} and R^{f4} are each —(CH₂)₂—. In some embodiments, R^{f3} and R^{f4} are each —(CH₂)₃—.

In some embodiments, R^{f4} is —(CH₂)₂—.

In some embodiments, R^{f5} is —(CH₂)₂—. In some embodiments, R^{f3} is —(CH₂)₃—. In some embodiments, R^{f5} is —(CH₂)₄—.

In some embodiments, R^{d1} and R^{e1} are each independently a linear C₁₋₁₂ alkyelene. In some embodiments, R^{d1} and R^{e1} are each independently C₅₋₁₀ alkylene. In some embodiments, R^{d1} and R^{c1} are each —(CH₂)₇—.

In some embodiments, X^{d} and X^{e} are each —C(O)O—*. In some embodiments, X^{d} and X^{e} are each —OC(O)—*.

In some embodiments, R^{d2} and R^{e2} are each a bond. In some embodiments, R^{d2} and R^{e2} are each —CH₂—.

In some embodiments, no more than one of R^{d3a}, R^{d3b}, R^{e3a}, and R^{e3b} is H. In some embodiments, R^{d3a}, R^{d3b}, R^{e3a}, and R^{e3b} are each independently a linear C₁₋₁₂ alkyl. In some embodiments, R^{d3a}, R^{d3b}, R^{e3a}, and R^{e3b} are each independently C₂₋₁₀ alkyl. In some embodiments, R^{d3a}, R^{d3b}, R^{e3a}, and R^{e3b} are each independently H or optionally substituted with a heterocyclyl comprising a disulfide bond. In some embodiments, R^{d3a}, R^{d3b}, R^{e3a}, and R^{e3b} are each independently H or optionally substituted with 1,2-dithiolanyl. In some embodiments, none of R^{d3a}, R^{d3b}, R^{e3a}, and R^{e3b} is H. In some embodiments, at least one of R^{d3} R^{d3b}, R^{e3d}, and R^{e3b} is substituted with a heterocyclyl. In some embodiments, at least one of R^{d3a}, R^{d3b}, R^{e3a}, and R^{e3b} is C₁₋₁₂ alkyl substituted with a 5- to 10-membered heterocyclyl comprising a disulfide bond or —(C₁₋₁₀ alkylene)-Sₙ—(C₁₋₁₀ alkyl).

In some embodiments, R^{d3} and R^{e3} are each independently

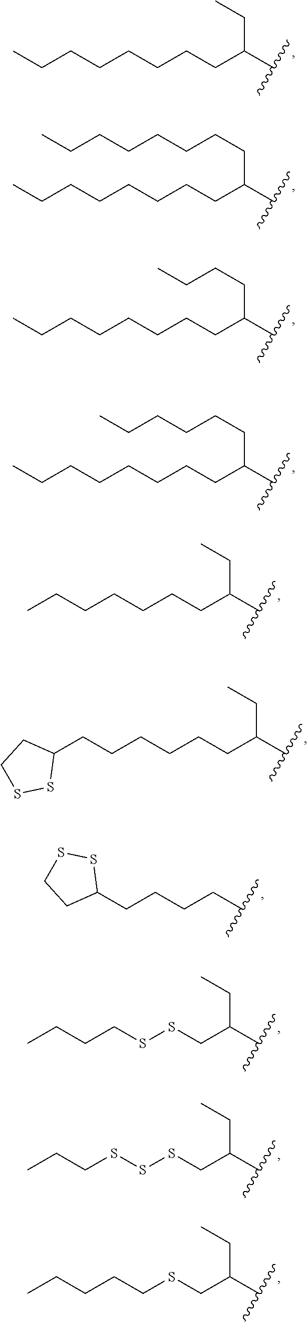

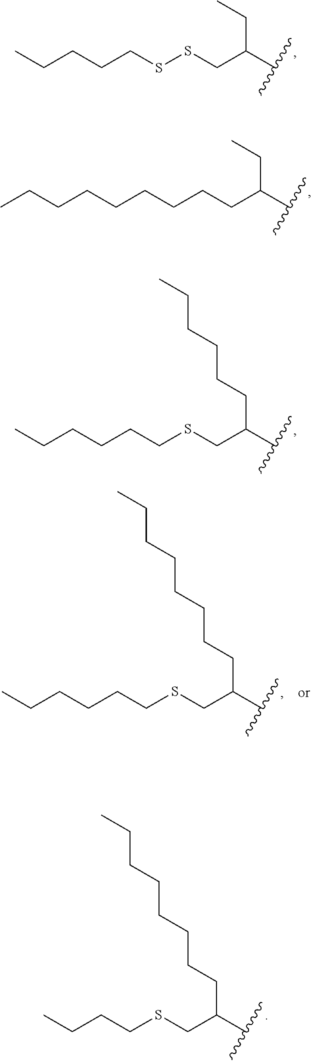 [SMILES: *C(CC)CSSCCCCC ; *C(CC)CCCCCCCCC ; *C(CCCCCC)CSCCCCCC ; *C(CCCCCCCC)CSCCCCCC ; *C(CCCCCCCC)CSCCCC]

In some embodiments, R^{d3} and R^{e3} are the same.

In some embodiments, the compound or the salt thereof is selected from the group consisting of the compounds of Table 1 and salts thereof.

In some aspects, provided is a lipid nanoparticle (LNP) comprising a lipid blend for targeted delivery of a nucleic acid into an immune cell, the lipid blend comprising a lipid-immune cell targeting group conjugate comprising the compound of Formula (II): [Lipid]-[optional linker]-[immune cell targeting group].

In some embodiments, the lipid blend further comprises an ionizable cationic lipid. In some embodiments, the ionizable cationic lipid comprises the compound described herein, or a salt thereof.

In some embodiments, the LNP further comprises a nucleic acid, wherein the nucleic acid is encapsulated in the LNP.

In some embodiments, the immune cell targeting group comprises an antibody that binds a T cell antigen. In some embodiments, the T cell antigen is CD3, CD4, CD7, CD8, or a combination thereof (e.g., both CD3 and CD8, both CD4 and CD8, or both CD7 and CD8).

In some embodiments, the immune cell targeting group comprises an antibody that binds a Natural Killer (NK) cell antigen. In some embodiments, the NK cell antigen is CD7, CD8, CD56, or a combination thereof (e.g., both CD7 and CD8).

In some embodiments, the immune cell targeting group comprises an antibody that binds a macrophage antigen, a monocyte antigen, and/or a dendritic antigen. In some embodiments, the macrophage comprises an M1 macrophage, an M2 macrophage, or both. In some embodiments, the macrophage comprises an M2a macrophage, an M2b macrophage, an M2c macrophage, or any combination thereof. In some embodiments, the macrophage antigen comprises CDIIB, CD68, CD80, CD86, TRL-2, TRL-4, INOS, MHC-II, CD163, CD206, CD209, FIZZ1, or Ym1/2, or any combination thereof. In some embodiments, the macrophage antigen comprises CD206.

In some embodiments, the immune cell targeting group is covalently coupled to a lipid in the lipid blend via a polyethylene glycol (PEG) containing linker. In some embodiments, the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the immune cell targeting group is covalently coupled to a lipid in the lipid blend via a distearoyl-phosphatidylethanolamine (DSPE).

In some embodiments, the PEG is PEG 2000 or PEG 3400. In some embodiments, the PEG is PEG 3400.

In some embodiments, the lipid-immune cell targeting group conjugate is present in the lipid blend in a range of 0.001 to 0.5 mole percent (e.g., 0.002-0.2 mole percent).

In some embodiments, the lipid blend further comprises one or more of a structural lipid, a neutral phospholipid, and a free PEG-lipid.

In some embodiments, the ionizable cationic lipid is present in the lipid blend in a range of 30-70 (e.g., 40-60) mole percent. In some embodiments, the ionizable cationic lipid is present in the lipid blend in a range of about 48 mol % to about 50 mol %. In some embodiments, the ionizable cationic lipid is present in the lipid blend in about 49.2 mol %.

In some embodiments, the structural lipid is sterol. In some embodiments, the sterol is cholesterol, fecosterol, β-sitosterol, ergosterol, campesterol, stigmasterol, stigmastanol, or brassicasterol. In some embodiments, the sterol is present in the lipid blend in a range of 20-70 (e.g., 30-50) mole percent. In some embodiments, the sterol is cholesterol. In some embodiments, the sterol is present in the lipid blend in a range of about 27 mol % to about 29 mol %. In some embodiments, the sterol is present in the lipid blend in about 28.3 mol %.

In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin. In some embodiments, the neutral phospholipid is 1,2-distearoyl-sn-glycero-3-phosphocholine (DSPC).

In some embodiments, the neutral phospholipid is present in the lipid blend in a range of 5-15 mol %. In some embodiments, the neutral phospholipid is present in the lipid blend in a range of about 16 mol % to about 40 mol %. In some embodiments, the neutral phospholipid is present in the lipid blend in a range of about 16 mol % to about 30 mol %. In some embodiments, the neutral phospholipid is present in the lipid blend in a range of about 16 mol % to about 25 mol %. In some embodiments, the neutral phospholipid is present in the lipid blend in a range of about 19 mol % to about 21 mol %. In some embodiments, the neutral phospholipid is present in the lipid blend in about 20 mol %.

In some embodiments, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid. In some embodiments, the free PEG-lipid comprises a diacylphosphatidylethanolamine comprising dimyristoyl (C14) chain, Dipalmitoyl (C16) chain or Distearoyl (C18) chain, and optionally the free PEG-lipid comprises PEG-DPG and PEG-DMG. In some embodiments, the free PEG-lipid is present in the lipid blend in a range of about 1 to about 4 mole percent, such as about 0.5 to about 2.5 mole percent. In some embodiments, the free PEG-lipid comprises the same or a different lipid as the lipid in the lipid-immune cell targeting group conjugate. In some embodiments, the free PEG-lipid is DPG-PEG2K. In some embodiments, the free PEG-lipid is present in the lipid blend in a range of about 2.4 mol % to about 2.6 mol %. In some embodiments, the free PEG-lipid is present in the lipid blend in about 2.5 mol %.

In some embodiments, the LNP has a mean diameter in the range of 50-200 nm. In some embodiments, the LNP has a mean diameter of between about 75 nm and about 80 nm. In some embodiments, the LNP has a polydispersity index in a range from about 0.01 to about 0.5. In some embodiments, the LNP has a pKa of between about 5.0 and about 8.0.

In some embodiments, the nucleic acid is DNA or RNA. In some embodiments, the RNA is an mRNA. In some embodiments, the mRNA encodes a receptor, a growth factor, a hormone, a cytokine, an antibody, an antigen, an enzyme, or a vaccine. In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR). In some embodiments, the mRNA encodes polypeptide capable of reprogramming an M2 macrophage to an M1 macrophage. In some embodiments, the RNA is ERNA, siRNA, gRNA, or microRNA.

In some embodiments, the immune cell targeting group comprises an antibody, and the antibody is a Fab or an immunoglobulin single variable (ISV) domain (e.g., a Nanobody). In some embodiments, the antibody is a human or humanized antibody. In some embodiments, the immune cell targeting group comprises a Fab, F(ab′)₂, Fab′-SH, Fv, or scFv fragment, or any combination thereof. In some embodiments, the immune cell targeting group comprises a Fab. In some embodiments, the Fab is engineered to knock out the natural interchain disulfide bond at the C-terminus. In some embodiments, the Fab comprises a heavy chain fragment that comprises C233S substitution, and a light chain fragment that comprises C214S substitution, numbering according to Kabat. In some embodiments, the Fab has a non-natural interchain disulfide bond (e.g., an engineered, buried interchain disulfide bond). In some embodiments, the Fab comprises F174C substitution in the heavy chain fragment, and S176C substitution in the light chain fragment, numbering according to Kabat. In some embodiments, the Fab comprises a cysteine at the C-terminus of the heavy or light chain fragment. In some embodiments, the Fab further comprises one or more amino acids between the heavy chain fragment of the Fab and the C-terminal cysteine. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker.

In some embodiments, the immune cell targeting group comprises an ISV domain. In some embodiments, the ISV domain is Nanobody® ISV. In some embodiments, the ISV domain comprises a cysteine at the C-terminus. In some embodiments, the ISV domain comprises a V_{HH} domain and further comprises a spacer comprising one or more amino acids between the V_{HH} domain and the C-terminal cysteine. In some embodiments, the immune cell targeting group comprises two or more V_{HH} domains. In some embodiments, the two or more V_{HH} domains are linked by an amino acid linker. In some embodiments, the immune cell targeting group comprises a first V_{HH} domain linked to an antibody CH1 domain and a second V_{HH} domain linked to an antibody light chain constant domain. In some embodiments, the antibody CH1 domain and the antibody light chain constant domain are linked by one or more disulfide bonds. In some embodiments, the immune cell targeting group comprises a Van domain linked to an antibody CH1 domain, and wherein the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat.

In some embodiments, the immune cell targeting group comprises a Fab that comprises:
(a) a heavy chain fragment comprising the amino acid sequence of SEQ ID NO: 1 and a light chain fragment comprising the amino acid sequence of SEQ ID NO: 2 or 3; or
(b) a heavy chain fragment comprising the amino acid sequence of SEQ ID NO: 6 and a light chain fragment comprising the amino acid sequence of SEQ ID NO: 7.

In some embodiments, the LNP is for delivering a nucleic acid into an immune cell, and wherein the immune cell is an NK cell, and the immune cell targeting group comprises an antibody that binds CD56. In some embodiments, the LNP is for delivering a nucleic acid into an immune cell, and wherein the immune cell targeting group comprises an antibody that binds CD7 or CD8, and the free PEG-lipid is DMG-PEG or PEG-DPG.

In some embodiments, the LNP is for delivering a nucleic acid into an immune cell, and wherein the immune cell is a macrophage, and the immune cell targeting group comprises an antibody that binds CD206. In some embodiments, the LNP is for delivering a nucleic acid into an immune cell, and wherein the immune cell targeting group comprises an antibody that binds CD206, and the free PEG-lipid is DMG-PEG or PEG-DPG.

In some embodiments, the free PEG-lipid comprises a PEG having a molecular weight of at least 2000 daltons. In some embodiments, the PEG has a molecular weight of about 3000 to 5000 daltons.

In some embodiments, the antibody is a Fab. In some embodiments, the Fab binds CD3, and the free PEG-lipid in the LNP comprises a PEG having a molecular weight of about 2000 daltons. In some embodiments, the Fab is an anti-CD4 antibody, and the free PEG-lipid in the LNP comprises a PEG having a molecular weight of about 3000 to 3500 daltons.

In some embodiments, the Fab binds CD206, and the free PEG-lipid in the LNP comprises a PEG having a molecular weight of about 2000 daltons. In some embodiments, the Fab is an anti-CD206 antibody, and the free PEG-lipid in the LNP comprises a PEG having a molecular weight of about 3000 to 3500 daltons.

In some embodiments, the LNP is for delivering a nucleic acid into an immune cell, and wherein the LNP binds CD3, and also binds CD11a or CD18 of the immune cell. In some embodiments, the LNP comprises two conjugates, wherein the first conjugate comprises an antibody that binds CD3, and the second conjugate comprises an antibody that binds CD11a or CD18. In some embodiments, the LNP comprises one conjugate, and the conjugate comprises a bispecific antibody that binds both CD3 and CD11a. In some embodiments, the LNP comprises one conjugate, and the conjugate comprises a bispecific antibody that binds both CD3 and CD18. In some embodiments, the bispecific antibody is an immunoglobulin single variable domain or Fab-ScFv.

In some embodiments, the LNP is for delivering a nucleic acid into an immune cell, and wherein the LNP binds CD7 and CD8 of the immune cell. In some embodiments, the LNP comprises two conjugates, wherein the first conjugate comprises an antibody that binds CD7, and a second conjugate that binds CD8. In some embodiments, the LNP comprises one conjugate, wherein the conjugate comprises a bispecific antibody that binds CD7 and CD8. In some embodiments, the bispecific antibody is an immunoglobulin single variable domain or Fab-ScFv.

In some embodiments, the LNP is for delivering a nucleic acid into an immune cell, and wherein the LNP binds a first macrophage antigen, and also binds a second macrophage antigen. In some embodiments, the LNP comprises two conjugates, wherein the first conjugate comprises an antibody that binds the first macrophage antigen, and the second conjugate comprises an antibody that binds the second macrophage antigen. In some embodiments, the LNP comprises one conjugate, and the conjugate comprises a bispecific antibody that binds both the first macrophage antigen and the second macrophage antigen. In some embodiments, the bispecific antibody is an immunoglobulin single variable domain or Fab-ScFv.

In some embodiments, the LNP binds to a first antigen on the surface of the first type of immune cell, and also binds to a second antigen on the surface of the second type of immune cell. In some embodiments, the two different types of immune cells are CD4+ T cells and CD8+ T cell. In some embodiments, the first type of immune cell is a first macrophage, and the second type of immune cell is a second macrophage, a T-cell, or an NK cell. In some embodiments, the LNP comprises two conjugates, and the first conjugate comprises a first antibody that binds to the first antigen of the first type of immune cell, and the second conjugate comprises a second antibody that binds to the second antigen of the second type of immune cell.

In some embodiments, the LNP comprises one conjugate, and the conjugate comprises a bispecific antibody, and the bispecific antibody binds to both the first antigen on the first type of immune cell, and the second antigen on the second type of immune cells. In some embodiments, the bispecific antibody is an immunoglobulin single variable domain or a Fab-ScFv.

In some embodiments, the LNP is for delivering a nucleic acid into an immune cell, and wherein the immune cell targeting group comprises a single antibody that binds to CD3 or CD7. In some embodiments, the LNP is for delivering a nucleic acid into an immune cell, and wherein the immune cell targeting group binds to CD7, CD8, or both CD7 and CD8.

In some embodiments, the LNP is for delivering a nucleic acid into both T cells and NK cells, wherein the immune cell targeting group binds to
(a) both CD3 and CD56;
(b) both CD8 and CD56; or
(c) both CD7 and CD56.

In some embodiments, the LNP is for delivering a nucleic acid into an immune cell, and wherein the immune cell targeting group comprises a Fab lacking the native interchain disulfide bond.

In some embodiments, the Fab is engineered to replace one or both cysteines on the native constant light chain and the native constant heavy chain that form the native interchain disulfide with a non-cysteine amino acid, therefor to remove the native interchain disulfide bond in the Fab.

In some aspects, provided is a method of targeting the delivery of a nucleic acid to an immune cell of a subject, the method comprising contacting the immune cell with the LNP described herein, wherein the LNP comprises the nucleic acid.

In some aspects, provided is a method of expressing a polypeptide of interest in a targeted immune cell of a subject, the method comprising contacting the immune cell with the LNP described herein, wherein the LNP comprises a nucleic acid encoding the polypeptide.

In some aspects, provided is a method of modulating cellular function of a target immune cell of a subject, the method comprising administering to the subject the LNP described herein, wherein the LNP comprises a nucleic acid modulates the cellular function of the immune cell.

In some aspects, provided is a method of treating, ameliorating, or preventing a symptom of a disorder or disease in a subject, the method comprising administering to the subject the LNP described herein for delivering a nucleic acid into an immune cell of the subject, wherein the LNP comprises the nucleic acid. In some aspects, provided is a method of treating, ameliorating, or preventing a symptom of a disorder or disease in a subject in need thereof, the method comprising administering to the subject an LNP described herein, wherein the LNP comprises a nucleic acid and delivers the nucleic acid into an immune cell of the subject.

In some embodiments, the disorder is an immune disorder, an inflammatory disorder, or cancer. In some embodiments, the nucleic acid encodes an antigen for use in a therapeutic or prophylactic vaccine for treating or preventing cancer or an infection by a pathogen. In some embodiments, no more than 5% non-immune cells are transfected by the LNP. In some embodiments, half-life of the nucleic acid delivered by the LNP or a polypeptide encoded by the nucleic acid delivered by the LNP is at least 10% longer than half-life of nucleic acid delivered by a reference LNP or a polypeptide encoded by the nucleic acid delivered by the reference LNP. In some embodiments, at least 10% immune cells are transfected by the LNP. In some embodiments, expression level of the nucleic acid delivered by the LNP is at least 10% higher than expression level of nucleic acid delivered by a reference LNP.

In some embodiments, the LNP comprises the compound described herein, or a salt thereof. In some embodiments, (i) R^{c3} is

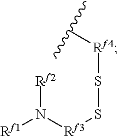

or (ii) R^{a3} and R^{b3} are each independently

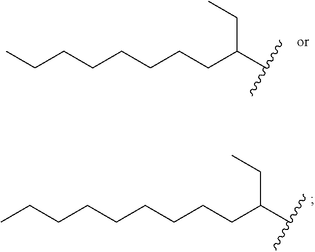 [SMILES: *C(CC)CCCCCCCC ; *C(CC)CCCCCCCCC]

or both (i) and (ii). In some embodiments, the LNP comprises Lipid 28, Lipid 6, Lipid 12, Lipid 1, or Lipid 7 of Table 1, or a salt of thereof, or any combination thereof. In some embodiments, (i) R^{a3} and R^{b3} are each independently

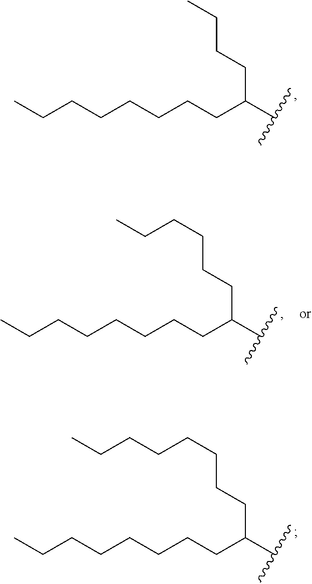 [SMILES: *C(CCCC)CCCCCCCC ; *C(CCCCCC)CCCCCCCC ; *C(CCCCCCCC)CCCCCCCC]

or (ii) R^{a3} and R^{b3} are each

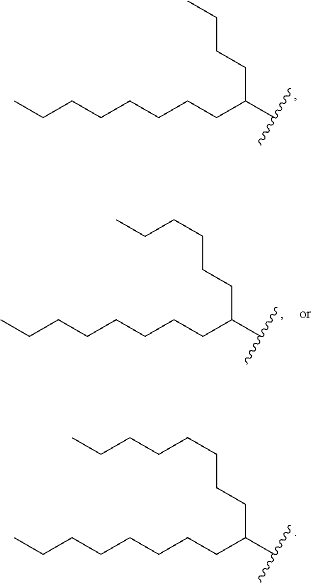 [SMILES: *C(CCCC)CCCCCCCC ; *C(CCCCCC)CCCCCCCC ; *C(CCCCCCCC)CCCCCCCC]

In some embodiments, the LNP comprises Lipid 9 or Lipid 10 of Table 1, or a salt thereof, or any combination thereof.

In some aspects, provided is a method of targeting the delivery of a nucleic acid to a non-liver cell, the method comprising contacting the non-liver cell with an LNP comprising the compound described herein, or a salt thereof, wherein (i) R^{c3} is

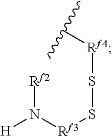

or (ii) R^{a3} and R^{b3} are each independently

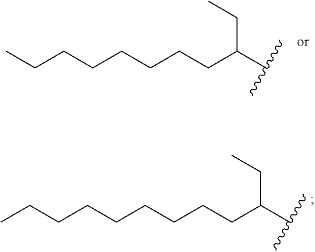 [SMILES: *C(CC)CCCCCCCC ; *C(CC)CCCCCCCCC]

or both (i) and (ii). In some embodiments, the LNP comprises Lipid 28, Lipid 6, Lipid 12, Lipid 1, or Lipid 7 of Table 1, or a salt of thereof, or any combination thereof.

In some aspects, provided is a method of targeting the delivery of a nucleic acid to a liver cell, the method comprising contacting the liver cell with an LNP comprising the compound described herein, or a salt thereof, wherein (i) R^{a3} and R^{b3} are each independently

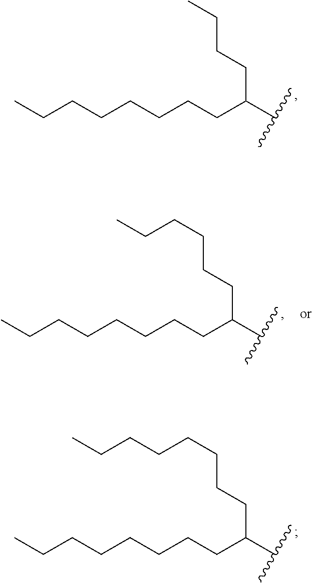 [SMILES: *C(CCCC)CCCCCCCC ; *C(CCCCCC)CCCCCCCC ; *C(CCCCCCCC)CCCCCCCC]

or at least one of R^{a3} and R^{b3} is

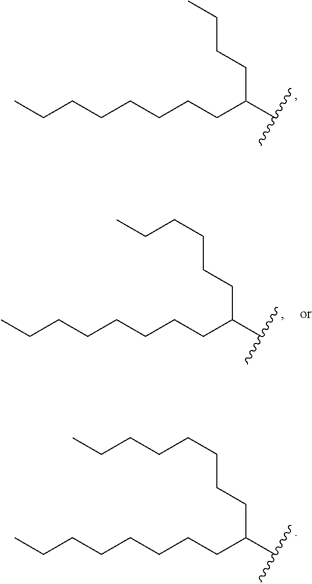 [SMILES: *C(CCCC)CCCCCCCC ; *C(CCCCCC)CCCCCCCC ; *C(CCCCCCCC)CCCCCCCC]

In some embodiments, the LNP comprises Lipid 9 or Lipid 10 of Table 1, or a salt thereof.

In some aspects, provided herein is a method of targeting the delivery of a nucleic acid to a placental cell, the method comprising contacting the placental cell with an LNP comprising the compound described herein.

In some aspects, provided herein is a method of targeting the delivery of a nucleic acid to a hematopoietic stem cell (HSC), the method comprising contacting the HSC with an LNP comprising the compound described herein. In some embodiments, the LNP comprises Lipid 1. In some embodiments, the LNP comprises Lipid 1, Lipid 12, or Lipid 53 of Table 1, or a salt thereof, or any combination thereof. Various aspects and embodiments of the invention are described in further detail below.

In some aspects, provided herein is a lipid nanoparticle (LNP) comprising a lipid blend for targeted delivery of a nucleic acid into a hematopoietic stem cell (HSC). In some embodiments, the lipid blend comprises a lipid-cell targeting group conjugate comprising the compound of Formula (V): [Lipid]-[optional linker]-[cell targeting group] and an ionizable cationic lipid described herein. In some embodiments, the cell targeting group is an antibody that binds to an antigen on the HSC. In some embodiments, the nucleic acid is encapsulated in the LNP.

In some embodiments, the antigen on the hematopoietic stem cell is selected from the group consisting of CD34, CD105, and CD117.

In some embodiments, the ionizable cationic lipid is present in the lipid blend in a range of 30-70 (e.g., 40-60) mol %. In some embodiments, the ionizable cationic lipid is present in the lipid blend in a range of about 48 mol % to about 50 mol %.

In some embodiments, the cell targeting group is covalently coupled to a lipid in the lipid blend via a polyethylene glycol (PEG) containing linker. In some embodiments, the lipid covalently coupled to the cell targeting group via a PEG containing linker is distearoylglycerol (DSG), distearoyl-phosphatidylethanolamine (DSPE), dimyristoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide. In some embodiments, the PEG is PEG 3400. In some embodiments, the lipid covalently coupled to the cell targeting group via a PEG containing linker is distearoyl-phosphatidylethanolamine (DSPE). In some embodiments, the [Lipid]-[optional linker]-[cell targeting group] conjugate is present in the lipid blend in a range of 0.001 to 0.5 mole percent (e.g., 0.002-0.2 mole percent).

In some embodiments, the lipid blend further comprises one or more of a structural lipid, a neutral phospholipid, and a free PEG-lipid.

In some embodiments, the structural lipid is present in the lipid blend in a range of 20-70 (e.g., 30-50) mol %. In some embodiments, the structural lipid is present in the lipid blend in a range of about 27 mol % to about 29 mol %. In some embodiments, the structural lipid is sterol. In some embodiments, the sterol is cholesterol, fecosterol, β-sitosterol, ergosterol, campesterol, stigmasterol, stigmastanol, or brassicasterol. In some embodiments, the sterol is cholesterol.

In some embodiments, the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-diolcoyl-sn-glycero-3-phosphocholine (DOPC), and sphingomyelin. In some embodiments, the neutral phospholipid is 1,2-distearoyl-sn-glycero-3-phosphocholine (DSPC). In some embodiments, the neutral phospholipid is present in the lipid blend in a range of 5-15 mol %. In some embodiments, the neutral phospholipid is present in the lipid blend in a range of about 16 mol % to about 40 mol %. In some embodiments, the neutral phospholipid is present in the lipid blend in a range of about 16 mol % to about 30 mol %. In some embodiments, the neutral phospholipid is present in the lipid blend in a range of about 16 mol % to about 25 mol %. In some embodiments, the neutral phospholipid is present in the lipid blend in a range of about 19 mol % to about 21 mol %. In some embodiments, the neutral phospholipid is present in the lipid blend in about 20 mol %.

In some embodiments, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, for example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyristoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid. In some embodiments, the free PEG-lipid is DPG-PEG2K. In some embodiments, the free PEG-lipid is present in the lipid blend in a range of about 1 to about 4 mol %, such as about 0.5 to about 3 mol %. In some embodiments, the free PEG-lipid is present in the lipid blend in a range of about 2.4 mol % to about 2.6 mol %.

In some embodiments, the cell targeting group comprises an antibody, and the antibody is a Fab or an immunoglobulin single variable (ISV) domain (e.g., a Nanobody). In some embodiments, the cell targeting group comprises a Fab, F(ab′)₂, Fab′-SH, Fv, or scFv fragment, or any combination thereof. In some embodiments, the cell targeting group comprises an ISV domain. In some embodiments, the cell targeting group comprises a Fab.

In some aspects, provided is a method of targeting the delivery of a nucleic acid to a hematopoietic stem cell (HSC). In some embodiments, the method comprises contacting the HSC with an LNP described herein. In some aspects, provided is a method of genetically modifying a hematopoietic stem cell (HSC), the method comprising contacting the HSC with an LNP described herein. In some aspects, provided is a method of treating a disease in a subject in need thereof, the method comprising administering to the subject an LNP described herein. In some embodiments, the LNP comprises a compound described herein. In some embodiments, the LNP comprises Lipid 1.

### BRIEF DESCRIPTION OF THE DRAWINGS

The present application can be understood by reference to the following description taken in conjunction with the accompanying figures.

FIG. 1 depicts the particle sizes of each formulation pre- and post-freeze/thaw. Medium for frozen samples was 25 mM HEPES, 75 mM NaCl, 5% sucrose, pH 7.4. Freeze/thaw test was performed by storing fresh 100 μg/mL of LNPs in −80° C. freezer at least overnight, and then thawing at room temperature right before measurement.

FIG. 2 depcits pKa values of LNPs of novel lipids measured by the acid-base titration method with 2(p-toluidino)-6-naphthalene sulfonic acid (TNS) method.

FIG. 3A depicts in vivo imaging of luciferase expression in mice that received LNPs in Example 7 after 6 hours and 24 hours.

FIG. 3B depicts in vivo absolute expression value of luciferase in liver in mice that received LNPs in Example 7 after 6 hours and 24 hours.

FIG. 3C depicts in vivo absolute expression value of luciferase in in spleen in mice that received LNPs in Example 7 after 6 hours and 24 hours.

FIG. 4A depicts ex vivo luciferase expression images in selected tissues 24 hours after dosing of LNPs.

FIG. 4B depicts ex vivo luciferase expression in average radiance 24 hours after dosing.

FIG. 4C depicts ex vivo spleen relative to liver expression 24 hours after dosing of LNPs.

FIG. 4D depicts in vivo luciferase expression in liver 24 hours after LNPs injection through tail vein (dotted line is the luciferase expression of control LNPs in liver, to which all liver expressions were normalized).

FIG. 5A depicts in vivo transfection efficiency as measured by % mCherry positive human hematopoietic stem cells (HSCs) in humanized mice that are untreated or treated with HSC-targeted LNPs coated with anti-CD117 Fab and comprising Formulation A (10 mol % DSPC) or Formulation B (20 mo % DSPC) by intravenous injection (1 mg/kg).

FIG. 5B depicts mCherry median fluorescence intensity of human hematopoietic stem cells (HSCs) in humanized mice that are untreated or treated with HSC-targeted LNPs coated with anti-CD117 Fab and comprising Formulation A (10 mol % DSPC) or Formulation B (20 mo % DSPC) by intravenous injection (1 mg/kg).

FIG. 5C depicts off-tissue targeting signal measured in the liver, spleen, and lung were lower in mice treated with LNPs comprising Formulation B (10 mol % DPSC) or Formulation B (20 mol % DPSC).

FIG. 6 depicts in vivo luciferase expression in average radiance in placenta and fetus 24 hours after dosing.

FIG. 7 depicts structures of various Fab, VHH(Nb), ScFv, Fab-ScFv and Fab-VHH hybrids.

### DETAILED DESCRIPTION

The invention provides ionizable cationic lipids, lipid-cell targeting group conjuates (e.g., lipid-immune cell targeting group conjugates), and lipid nanoparticle compositions comprising such ionizable cationic lipids and/or lipid-cell (e.g., T-cell or hematopoetic stem cell) targeting group conjugates, medical kits comprising such lipids and/or conjugates, and methods of making and using, such lipids and conjugates.

The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

Where individual embodiments are disclosed, it should be appreciated that this disclosure is not limiting and that all embodiments may be combined. It should also be noted that references to methods or methods of treatment herein should be read as equivalent to compounds and/or compositions for use in said methods or methods of treatment.

Throughout this application, unless the context indicates otherwise, references to a compound of Formula (I) includes all subgroups of Formula (I) defined herein, such as Formula (I-P1) and (I-P2), including all substructures, subgenera, preferences, embodiments, examples and particular compounds defined and/or described herein.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts. In addition, when a chemical group is a diradical, for example, it is understood a that the chemical groups can be bonded to their adjacent atoms in the remainder of the structure in one or both orientations, for example, —OC(O)— is interchangeable with —C(O)O— or —OC(S)— is interchangeable with —C(S)O—.

The terms “a” and “an” as used herein mean “one or more” and include the plural unless the context is inappropriate. In some embodiments, “one or more” is 1 or 2. In some embodiments, “one or more” is 1, 2, or 3. In some embodiments, “one or more” is 1, 2, 3, or 4. In some embodiments, “one or more” is 1, 2, 3, 4, or 5. In some embodiments, “one or more” is 1, 2, 3, 4, 5, or more.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, or C₁-C₆alkyl, respectively. In some embodiments, alkyl is optionally substituted. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. In some embodiments, alkylene is optionally substituted. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

“Alkenyl” refers to an unsaturated branched or straight-chain alkyl group having the indicated number of carbon atoms (e.g., 2 to 8, or 2 to 6 carbon atoms) and at least one carbon-carbon double bond. The group may be in either the cis or trans configuration (Z or E configuration) about the double bond(s). Alkenyl groups include, but are not limited to, ethenyl, propenyl (e.g., prop-1-en-1-yl, prop-1-en-2-yl, prop-2-en-1-yl(allyl), prop-2-en-2-yl), and butenyl (e.g., but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl).

“Alkynyl” refers to an unsaturated branched or straight-chain alkyl group having the indicated number of carbon atoms (e.g., 2 to 8 or 2 to 6 carbon atoms) and at least one carbon-carbon triple bond. Alkynyl groups include, but are not limited to, ethynyl, propynyl (e.g., prop-1-yn-1-yl, prop-2-yn-1-yl) and butynyl (e.g., but-1-yn-1-yl, but-1-yn-3-yl, but-3-yn-1-yl).

The term “oxo” is art-recognized and refers to a “═O” substituent. For example, a cyclopentane substituted with an oxo group is cyclopentanone.

The term “morpholinyl” refers to a substituent having the structure of:

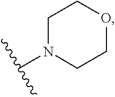 [SMILES: *N1CCOCC1]

which is optionally substituted.

The term “piperidinyl” refers to a substituent having a structure of:

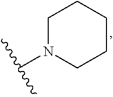 [SMILES: *N1CCCCC1]

which is optionally substituted.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, “optionally substituted” is equivalent to “unsubstituted or substituted.” In some embodiments, “optionally substituted” indicates that the designated atom or group is optionally substituted with one or more substituents independently selected from optional substituents provided herein. In some embodiments, optional substituent may be selected from the group consisting of: C₁₋₆alkyl, cyano, halogen, —O—C₁₋₆alkyl, C₁₋₆haloalkyl, C₃₋₇cycloalkyl, 3- to 7-membered heterocyclyl, 5- to 6-membered heteroaryl, and phenyl. In some embodiments, optional substituent is alkyl, cyano, halogen, halo, azide, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl, or heteroaryl. In some embodiments, optional substituent is —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R¹¹, —NR^{s12}C(O)R^{s13}, or —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

The term “haloalkyl” refers to an alkyl group that is substituted with at least one halogen. For example, —CH₂F, —CHF₂, —CF₃, —CH₂CF₃, —CF₂CF₃, and the like.

The term “cycloalkyl” refers to a monovalent saturated cyclic, bicyclic, bridged cyclic (e.g., adamantyl), or spirocyclic hydrocarbon group of 3-12, 3-10, 3-8, 4-8, or 4-6 carbons, referred to herein, e.g., as “C₄₋₈cycloalkyl,” derived from a cycloalkane. In some embodiments, cycloalkyl is optionally substituted. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. In some embodiments, heterocyclyl is optionally substituted. The number of ring atoms in the heterocyclyl group can be specified using Cₓ-Cₓ nomenclature where x is an integer specifying the number of ring atoms. For example, a C₃₋₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may be, for example, mono-, bi-, or other multi-cyclic ring systems (e.g., fused, spiro, bridged bicyclic). A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. In certain embodiments, the heterocyclyl group is 1,2-dithiolanyl. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “aryl” is art-recognized and refers to a carbocyclic aromatic group. In some embodiments, aryl is optionally substituted. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6- to 10-membered ring structure. In some embodiments, the aryl group is a C₆-C₁₄ aryl.

The term “heteroaryl” is art-recognized and refers to aromatic groups that include at least one ring heteroatom. In some embodiments, heteroaryl is optionally substituted. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula-N(R₁₀)(R¹¹), wherein R¹⁰ and R¹¹ each independently represent hydrogen, alkyl, cycloalkyl, heterocyclyl, alkenyl, aryl, aralkyl, or (CH₂)ₘ—R¹²; or R¹⁰ and R¹¹, taken together with the N atom to which they are attached complete a heterocycle having from 4 to 8 atoms in the ring structure; R¹² represents an aryl, a cycloalkyl, a cycloalkenyl, a heterocycle or a polycycle; and m is zero or an integer in the range of 1 to 8. In certain embodiments, R¹⁰ and R¹¹ each independently represent hydrogen, alkyl, alkenyl, or —(CH₂)ₘ—R¹².

The terms “alkoxyl” or “alkoxy” are art-recognized and refer to an alkyl group, as defined above, having an oxygen radical attached thereto. In some embodiments, alkoxyl is optionally substituted. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, O-alkynyl, —O—(CH₂)ₘ—R¹², where m and R¹² are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise,

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol “==” denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with 3H and 14C) are useful in compound and/or substrate tissue distribution assays. Tritiated (i.e., 3H) and carbon-14 (i.e., 14C) isotopes are particularly preferred for their ease of preparation and detectability. Further, substitution with heavier isotopes such as deuterium (i.e., 2H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable excipient” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Remington's The Science and Practice of Pharmacy, 21st Edition, A. R. Gennaro; Lippincott, Williams & Wilkins, Baltimore, MD, 2006.

As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

Abbreviations as used herein include diisopropylethylamine (DIPEA); 4-dimethylaminopyridine (DMAP); tetrabutylammonium iodide (TBAI); 1-ethyl-3-(3-dimethylaminopropyl) carbodiimide (EDC); benzotriazol-1-yl-oxytripyrrolidinophosphonium hexafluorophosphate (PyBOP), 9-Fluorenylmethoxycarbonyl (Fmoc), tetrabutyldimethylsilyl chloride (TBDMSCl), hydrogen fluoride (HF), phenyl (Ph), bis(trimethylsilyl)amine (HMDS), dimethylformamide (DMF); methylene chloride (DCM); tetrahydrofuran (THF); high-performance liquid chromatography (HPLC); mass spectrometry (MS), evaporative light scattering detector (ELSD), electrospray (ES)); nuclear magnetic resonance spectroscopy (NMR).

As used herein, the term “effective amount” refers to the amount of a compound (e.g., a nucleic acid, e.g., an mRNA) sufficient to effect beneficial or desired results. An effective amount can be administered in one or more administrations, applications or dosages and is not intended to be limited to a particular formulation or administration route. The term effective amount can be considered to include therapeutically and/or prophylactically effective amounts of a compound.

The phrase “therapeutically effective amount” as used herein means that amount of a compound (e.g., a nucleic acid, e.g., an mRNA), material, or composition comprising a compound (e.g., a nucleic acid, e.g., an mRNA) which is effective for producing some desired therapeutic effect in at least a sub-population of cells in a mammal, for example, a human, or a subject (e.g., a human subject) at a reasonable benefit/risk ratio applicable to any medical treatment.

The phrase “prophylactically effective amount” as used herein means that amount of a compound (e.g., a nucleic acid, e.g., an mRNA), material, or composition comprising a compound (e.g., a nucleic acid, e.g., an mRNA) which is effective for producing some desired prophylactic effect in at least a sub-population of cells in a mammal, for example, a human, or a subject (e.g., a human subject) by reducing, minimizing or eliminating the risk of developing a condition or the reducing or minimizing severity of a condition at a reasonable benefit/risk ratio applicable to any medical treatment.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As used herein, unless otherwise indicated, the term “antibody” means any antigen-binding molecule or molecular complex comprising at least one complementarity determining region (CDR) that specifically binds to or interacts with a particular antigen. It is understood the term encompasses an intact antibody (e.g., an intact monoclonal antibody), or a fragment thereof, such as an Fc fragment of an antibody (e.g., an Fe fragment of a monoclonal antibody), or an antigen-binding fragment of an antibody (e.g., an antigen-binding fragment of a monoclonal antibody), including an intact antibody, antigen-binding fragment, or Fc fragment that has been modified or engineered. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). The term also encompasses an immunoglobulin single variable domain, such as a Nanobody (e.g., a V_{HH}). The numbering of amino acid residues in antibodies disclosed herein is according to Kabat, unless otherwise explicitly stated.

As used here, an “antibody that binds to X” (i.e., X being a particular antigen), or “an anti-X antibody”, is an antibody that specifically recognizes the antigen X.

As used herein, a “buried interchain disulfide bond” or an “interchain buried disulfide bond” refers to a disulfide bond on a polypeptide which is not readily accessible to water soluble reducing agents, or is effectively “buried” in the hydrophobic regions of the polypeptide, such that it is unavailable to both reducing agents and for conjugation to other hydrophilic PEGs. Buried interchain disulfide bonds are further described in WO2017096361A1, which is incorporated by reference in its entirety.

As used herein, specificity of the targeted delivery by an LNP is defined by the ratio between % of a desired cell type (e.g., immune cell type or hematopoietic stem cell) that receives the delivered nucleic acid (e.g., on-target delivery), and % of an undesired cell type that is not meant to be the destination of the delivery, but receives the delivered nucleic acid (e.g., off-target delivery). For example, the specificity is higher when more desired cells receive the delivered nucleic acid, while less undesired cells receive the delivered nucleic acid. Specificity of the targeted delivery by an LNP can also be defined the ratio of amount of nucleic acid being delivered to the desired cells (e.g., on-target delivery) and amount of nucleic acid being delivered to the undesired cells (e.g., off-target delivery). Specificity of the delivery can be determined using any suitable method. As a non-limiting example, expression level of the nucleic acid in the desired cell type can be measured and compared to that of a different cell type that is not meant to be the destination of the delivery.

As used herein, in some embodiments, a reference LNP is an LNP that does not have the cell targeting group but is otherwise the same as the tested LNP. In some other embodiments, a reference LNP is an LNP that has a different ionizable cationic lipid but is otherwise the same as the tested LNP. In some embodiments, a reference LNP comprises D-Lin-MC3-DMA as the ionizable cationic lipid which is different from the ionizable cationic lipid in a tested LNP, but is otherwise the same as the tested LNP.

As used herein, a humanized antibody is an antibody which is wholly or partially of non-human origin and whose protein sequence has been modified to replace certain amino acids, for instance that occur at the corresponding position(s) in the framework regions of the VH and VL domains in a sequence of antibody from a human being, to increase its similarity to antibodies produced naturally in humans, in order to avoid or minimize an immune response in humans. For example, using techniques of genetic engineering, the variable domains of a non-human antibodies of interest may be combined with the constant domains of human antibodies. The constant domains of a humanized antibody are most of the time human CH and CL domains.

As used herein, the term “structural lipid” refers to sterols and also to lipids containing sterol moieties.

The “percent identity” between two sequences is a function of the number of identical positions shared by the sequences (i.e., % identity=number of identical positions/total number of positions×100), taking into account the number of gaps, and the length of each gap, which need to be introduced for optimal alignment of the two sequences. The percent identity between two amino acid sequences may be determined using the algorithm of E. Meyers and W. Miller (Comput. Appl. Biosci. 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (Mol. Biol. 48:444-453 (1970)) algorithm which has been incorporated into the GAP program in the GCG software package (available at www geg com), using either a Blossum 62 matrix or a PAM250 matrix, and a gap weight of 16, 14, 12, 10, 8, 6, or 4 and a length weight of 1, 2, 3, 4, 5, or 6.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### Immunoglobulin Single Variable Domain

In some embodiments, the cell targeting group of the LNPs as described herein comprise an immunoglobulin single variable domain, such as an Nanobody.

The term “immunoglobulin single variable domain” (ISV), interchangeably used with “single variable domain,” defines immunoglobulin molecules wherein the antigen binding site is present on, and formed by, a single immunoglobulin domain. This sets immunoglobulin single variable domains apart from “conventional” immunoglobulins (e.g., monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}—V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single V_{HH} or single V_{L} domain. Hence, the antigen binding site of an immunoglobulin single variable domain is formed by no more than three CDRs.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof, as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISV) can for example be a heavy chain ISV, such as a V_{H}, V_{HH}, including a camelized V_{H} or humanized V_{HH}. In one embodiment, it is a V_{HH}, including a camelized V_{H} or humanized V_{HH}. Heavy chain ISVs can be derived from a conventional four-chain antibody or from a heavy chain antibody.

For example, the immunoglobulin single variable domain may be a (single) domain antibody (or an amino acid sequence that is suitable for use as a single domain antibody), a “dAb” or dAb (or an amino acid sequence that is suitable for use as a dAb) or a Nanobody® ISV (as defined herein and including but not limited to a V_{HH}); other single variable domains, or any suitable fragment of any one thereof.

In particular, the immunoglobulin single variable domain may be a Nanobody® ISV (such as a V_{HH}, including a humanized V_{HH} or camelized V_{H}) or a suitable fragment thereof. [Note: Nanobody® is a registered trademark of Ablynx N.V.].

“Van domains”, also known as V_{HH}S, VAR antibody fragments, and VAR antibodies, have originally been described as the antigen binding immunoglobulin variable domain of “heavy chain antibodies” (i.e., of “antibodies devoid of light chains”; Hamers-Casterman et al. 1993 (Nature 363:446-448). The term “V_{HH} domain” has been chosen in order to distinguish these variable domains from the heavy chain variable domains that are present in conventional 4-chain antibodies (which are referred to herein as “Vu domains”) and from the light chain variable domains that are present in conventional 4-chain antibodies (which are referred to herein as “V_{L} domains”). For a further description of V_{HH}'s, reference is made to the review article by Muyldermans 2001 (Reviews in Molecular Biotechnology 74:277-302).

For the term “dAb's” and “domain antibody”, reference is for example made to Ward et al. 1989 (Nature 341:544), to Holt et al. 2003 (Trends Biotechnol. 21:484); as well as to for example WO 2004/068820, WO 2006/030220, WO 2006/003388 and other published patent applications of Domantis Ltd. It should also be noted that, although less preferred in the context of the present invention because they are not of mammalian origin, single variable domains can be derived from certain species of shark (for example, the so-called “IgNAR domains”, see for example WO 2005/18629).

Typically, the generation of immunoglobulins involves the immunization of experimental animals, fusion of immunoglobulin producing cells to create hybridomas and screening for the desired specificities. Alternatively, immunoglobulins can be generated by screening of naïve, immune or synthetic libraries, e.g., by phage display.

The generation of immunoglobulin sequences, such as VHHs, has been described extensively in various publications, among which WO 1994/04678, Hamers-Casterman et al. 1993 (Nature 363:446-448) and Muyldermans et al. 2001 (Reviews in Molecular Biotechnology 74:277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be used herein. Also, fully human, humanized or chimeric sequences can be used in the method described herein. For example, camelid immunoglobulin sequences and humanized camelid immunoglobulin sequences, or camelized domain antibodies, e.g., camelized dAb as described by Ward et al. 1989 (Nature 341:544), WO 1994/04678, and Davis and Riechmann (1994, Febs Lett., 339:285-290; and 1996, Prot. Eng., 9:531-537) can be used herein. Moreover, the ISVs are fused forming a multivalent and/or multispecific construct (for multivalent and multispecific polypeptides comprising one or more V_{HH} domains and their preparation, reference is also made to Conrath et al. 2001 (J. Biol. Chem., Vol. 276, 10. 7346-7350) as well as to for example WO 1996/34103 and WO 1999/23221).

A “humanized Van” comprises an amino acid sequence that corresponds to the amino acid sequence of a naturally occurring V_{HH} domain, but that has been “humanized”, i.e. by replacing one or more amino acid residues in the amino acid sequence of said naturally occurring VAR sequence (and in particular in the framework sequences) by one or more of the amino acid residues that occur at the corresponding position(s) in a Vu domain from a conventional 4-chain antibody from a human being (e.g., indicated above). This can be performed in a manner known per se, which will be clear to the skilled person, for example on the basis of the prior art (e.g., WO 2008/020079). Again, it should be noted that such humanized V_{HH}s can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a (camelid) heavy chain antibody. This can be performed in a manner known per se, which will be clear to the skilled person, for example on the basis of the description in the prior art (e.g., Davies and Riechman 1994, FEBS 339:285; 1995, Biotechnol. 13:475; 1996, Prot. Eng. 9:531; and Riechman 1999, J. Immunol. Methods 231:25). Such “camelizing” substitutions are inserted at amino acid positions that form and/or are present at the V_{H}—V_{L} interface, and/or at the so-called Camelidae hallmark residues, as defined herein (see for example WO 1994/04678 and Davies and Riechmann (1994 and 1996, supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g., a V_{L}-sequence) and/or from a heavy chain variable domain (e.g., a V_{H}-sequence or V_{HH} sequence). In one particular aspect, the framework sequences are either framework sequences that have been derived from a V_{HH}-sequence (in which said framework sequences may optionally have been partially or fully humanized) or are conventional V_{H} sequences that have been camelized (as defined herein).

In particular, the framework sequences present in the ISV sequence described herein may comprise one or more of hallmark residues (as defined herein), such that the ISV sequence is a Nanobody® ISV, such as, e.g., a V_{HH}, including a humanized V_{HH} or camelized V_{H}. Non-limiting examples of (suitable combinations of) such framework sequences will become clear from the further disclosure herein.

The total number of amino acid residues in a V_{H} domain and a V_{HH} domain will usually be in the range of from 110 to 120, often between 112 and 115. It should however be noted that smaller and longer sequences may also be suitable for the purposes described herein.

However, it should be noted that the ISVs described herein is not limited as to the origin of the ISV sequence (or of the nucleotide sequence used to express it), nor as to the way that the ISV sequence or nucleotide sequence is (or has been) generated or obtained. Thus, the ISV sequences may be naturally occurring sequences (from any suitable species) or synthetic or semi-synthetic sequences. In a specific but non-limiting aspect, the ISV sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g., DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, Nanobody® ISVs (in particular Vau sequences, including (partially) humanized V_{HH} sequences and camelized V_{H} sequences) can be characterized by the presence of one or more “Hallmark residues” (as described herein) in one or more of the framework sequences (again as further described herein). Thus, generally, a Nanobody® ISV can be defined as an immunoglobulin sequence with the (general) structure

### FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISV can be an immunoglobulin sequence with the (general) structure

### FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody® ISV can be an immunoglobulin sequence with the (general) structure

### FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and in which: one or more of the amino acid residues at positions 11, 37, 44, 45, 47, 83, 84, 103, 104 and 108 according to the Kabat numbering are chosen from the Hallmark residues mentioned in Table A below.

TABLE A
Hallmark Residues in Nanobody ® ISVs

| Position | Human V_{H}3 | Hallmark Residues |
|---|---|---|
| 11 | L, V; predominantly L | L, S, V, M, W, F, T, Q, E, A, R, G, K, Y, N, P, I; |
| | | preferably L |
| 37 | V, I, F; usually V | F⁽¹⁾, Y, V, L, A, H, S, I, W, C, N, G, D, T, P, preferably |
| | | F⁽¹⁾ or Y |
| 44⁽⁸⁾ | G | E⁽³⁾, Q⁽³⁾, G⁽²⁾, D, A, K, R, L, P, S, V, H, T, N, W, M, I; |
| | | preferably G⁽²⁾, E⁽³⁾ or Q⁽³⁾; most preferably G⁽²⁾ or Q⁽³⁾. |
| 45⁽⁸⁾ | L | L⁽²⁾, R⁽³⁾, P, H, F, G, Q, S, E, T, Y, C, I, D, V; |
| | | preferably L⁽²⁾ or R⁽³⁾ |
| 47⁽⁸⁾ | W, Y | F⁽¹⁾, L⁽¹⁾ or W⁽²⁾ G, I, S, A, V, M, R, Y, E, P, T, C, H, |
| | | K, Q, N, D; preferably W^{(2),} L⁽¹⁾ or F⁽¹⁾ |
| 83 | R or K; usually R | R, K⁽⁵⁾, T, E⁽⁵⁾, Q, N, S, I, V, G, M, L, A, D, Y, H; |
| | | preferably K or R; most preferably K |
| 84 | A, T, D; predominantly A | P⁽⁵⁾, S, H, L, A, V, I, T, F, D, R, Y, N, Q, G, E; |
| | | preferably P |
| 103 | W | W⁽⁴⁾, R⁽⁶⁾, G, S, K, A, M, Y, L, F, T, N, V, Q, P⁽⁶⁾, E, |
| | | C; preferably W |
| 104 | G | G, A, S, T, D, P, N, E, C, L; preferably G |
| 108 | L, M or T; predominantly | Q, L⁽⁷⁾, R, P, E, K, S, T, M, A, H; preferably Q or L⁽⁷⁾ |
| | L | |

Notes:
In particular, but not exclusively, in combination with KERE (SEQ ID NO: 103) or KQRE (SEQ ID NO: 104) at positions 43-46.
Usually as GLEW (SEQ ID NO: 105) at positions 44-47.
Usually as KERE (SEQ ID NO: 103) or KQRE (SEQ ID NO: 104) at positions 43-46, e.g., as KEREL (SEQ ID NO: 106), KEREF (SEQ ID NO: 107), KQREL (SEQ ID NO: 108), KQREF (SEQ ID NO: 109), KEREG (SEQ ID NO: 110), KQREW (SEQ ID NO: 111) or KQREG (SEQ ID NO: 112) at positions 43-47. Alternatively, also sequences such as TERE (SEQ ID NO: 113) (for example TEREL (SEQ ID NO: 114)), TQRE (SEQ ID NO: 115) (for example TQREL (SEQ ID NO: 116)), KECE (SEQ ID NO: 117) (for example KECEL (SEQ ID NO: 118) or KECER (SEQ ID NO: 119)), KQCE (SEQ ID NO: 120) (for example KQCEL (SEQ ID NO: 121)), RERE (SEQ ID NO: 122) (for example REREG (SEQ ID NO: 123)), RQRE (SEQ ID NO: 124) (for example RQREL (SEQ ID NO: 125), RQREF (SEQ ID NO: 126) or RQREW (SEQ ID NO: 127)), QERE (SEQ ID NO: 128) (for example QEREG (SEQ ID NO: 129)), QQRE (SEQ ID NO: 130), (for example QQREW (SEQ ID NO: 131), QQREL (SEQ ID NO: 132) or QQREF (SEQ ID NO: 133)), KGRE (SEQ ID NO: 134) (for example KGREG (SEQ ID NO: 135)), KDRE (SEQ ID NO: 136) (for example KDREV (SEQ ID NO: 137)) are possible. Some other possible, but less preferred sequences include for example DECKL (SEQ ID NO: 138) and NVCEL (SEQ ID NO: 139).
With both GLEW (SEQ ID NO: 105) at positions 44-47 and KERE (SEQ ID NO: 103) or KQRE (SEQ ID NO: 104) at positions 43-46.
Often as KP or EP at positions 83-84 of naturally occurring V_{HH} domains.
In particular, but not exclusively, in combination with GLEW (SEQ ID NO: 105) at positions 44-47.
With the proviso that when positions 44-47 are GLEW (SEQ ID NO: 105), position 108 is always Q in (non-humanized) V_{HH} sequences that also contain a W at 103.
The GLEW group also contains GLEW-like sequences at positions 44-47, such as for example GVEW (SEQ ID NO: 140), EPEW (SEQ ID NO: 141), GLER (SEQ ID NO: 142), DQEW (SEQ ID NO: 143), DLEW (SEQ ID NO: 144), GIEW (SEQ ID NO: 145), ELEW (SEQ ID NO: 146), GPEW (SEQ ID NO: 147), EWLP (SEQ ID NO: 148), GPER (SEQ ID NO: 149), GLER (SEQ ID NO: 142) and ELEW (SEQ ID NO: 146).

In one embodiment, the immunoglobulin single variable domain has certain amino acid substitutions in the framework regions effective in preventing or reducing binding of so-called “pre-existing antibodies” to the polypeptides. ISVs in which (i) the amino acid residue at position 112 is one of K or Q; and/or (ii) the amino acid residue at position 89 is T; and/or (iii) the amino acid residue at position 89 is L and the amino acid residue at position 110 is one of K or Q; and (iv) in each of cases (i) to (iii), the amino acid at position 11 is preferably V have been described in WO2015/173325.

### Polypeptides

The immunoglobulin single variable domains may form part of a protein or polypeptide, which may comprise or essentially consist of one or more (at least one) immunoglobulin single variable domains and which may optionally further comprise one or more further amino acid sequences (all optionally linked via one or more suitable linkers). The term “immunoglobulin single variable domain” may also encompass such polypeptides. The one or more immunoglobulin single variable domains may be used as a binding unit in such a protein or polypeptide, which may optionally comprise one or more further amino acids that can serve as a binding unit, so as to provide a monovalent, multivalent or multispecific polypeptide of the invention, respectively (for multivalent and multispecific polypeptides comprising one or more VHH domains and their preparation, reference is also made to Conrath et al. 2001 (J. Biol. Chem. 276:7346), as well as to for example WO 1996/34103, WO 1999/23221 and WO 2010/115998).

The polypeptides may comprise or essentially consist of one immunoglobulin single variable domain, as outlined above. Such polypeptides are also referred to herein as monovalent polypeptides.

The term “multivalent” indicates the presence of multiple ISVs in a polypeptide. In one embodiment, the polypeptide is “bivalent”, i.e., comprises or consists of two ISVs. In one embodiment, the polypeptide is “trivalent”, i.e., comprises or consists of three ISVs. In another embodiment, the polypeptide is “tetravalent”, i.e. comprises or consists of four ISVDs. The polypeptide can thus be “bivalent”, “trivalent”, “tetravalent”, “pentavalent”, “hexavalent”, “heptavalent”, “octavalent”, “nonavalent”, etc., i.e., the polypeptide comprises or consists of two, three, four, five, six, seven, eight, nine, etc., ISVs, respectively. In one embodiment the multivalent ISV polypeptide is trivalent. In another embodiment the multivalent ISV polypeptide is tetravalent. In still another embodiment, the multivalent ISV polypeptide is pentavalent.

In one embodiment, the multivalent ISV polypeptide can also be multispecific. The term “multispecific” refers to binding to multiple different target molecules (also referred to as antigens). The multivalent ISV polypeptide can thus be “bispecific”, “trispecific”, “tetraspecific”, etc., i.e., can bind to two, three, four, etc., different target molecules, respectively.

For example, the polypeptide may be bispecific-trivalent, such as a polypeptide comprising or consisting of three ISVs, wherein two ISVs bind to a first target and one ISV binds to a second target different from the first target. In another example, the polypeptide may be trispecific-tetravalent, such as a polypeptide comprising or consisting of four ISVs, wherein one ISV binds to a first target, two ISVs bind to a second target different from the first target and one ISV binds to a third target different from the first and the second target. In still another example, the polypeptide may be trispecific-pentavalent, such as a polypeptide comprising or consisting of five ISVs, wherein two ISVs bind to a first target, two ISVs bind to a second target different from the first target and one ISV binds to a third target different from the first and the second target.

In one embodiment, the multivalent ISV polypeptide can also be multiparatopic. The term “multiparatopic” refers to binding to multiple different epitopes on the same target molecules (also referred to as antigens). The multivalent ISV polypeptide can thus be “biparatopic”, “triparatopic”, etc., i.e., can bind to two, three, etc., different epitopes on the same target molecules, respectively.

In another aspect, the polypeptide of the invention that comprises or essentially consists of one or more immunoglobulin single variable domains (or suitable fragments thereof), may further comprise one or more other groups, residues, moieties or binding units. Such further groups, residues, moieties, binding units or amino acid sequences may or may not provide further functionality to the immunoglobulin single variable domain (and/or to the polypeptide in which it is present) and may or may not modify the properties of the immunoglobulin single variable domain.

For example, such further groups, residues, moieties or binding units may be one or more additional amino acids, such that the compound, construct or polypeptide is a (fusion) protein or (fusion) polypeptide. In a preferred but non-limiting aspect, said one or more other groups, residues, moieties or binding units are immunoglobulins. Even more preferably, said one or more other groups, residues, moieties or binding units are chosen from the group consisting of domain antibodies, amino acids that are suitable for use as a domain antibody, single domain antibodies, amino acids that are suitable for use as a single domain antibody, “dAb” s, amino acids that are suitable for use as a dAb, or Nanobodies.

Alternatively, such groups, residues, moieties or binding units may for example be chemical groups, residues, moieties, which may or may not by themselves be biologically and/or pharmacologically active. For example, and without limitation, such groups may be linked to the one or more immunoglobulin single variable domain so as to provide a “derivative” of the immunoglobulin single variable domain.

In another embodiment, said further residues may be effective in preventing or reducing binding of so-called “pre-existing antibodies” to the polypeptides. For this purpose, the polypeptides and constructs may comprise a C-terminal extension (X)n (in which n is 1 to 10, preferably 1 to 5, such as 1, 2, 3, 4 or 5 (and preferably 1 or 2, such as 1); and each X is an (preferably naturally occurring) amino acid residue that is independently chosen, and preferably independently chosen from the group consisting of alanine (A), glycine (G), valine (V), leucine (L) or isoleucine (I), for which reference is made to WO 2012/175741. Accordingly, the polypeptide may further comprise a C-terminal extension (X)n, in which n is 1 to 5, such as 1, 2, 3, 4 or 5, and in which X is a naturally occurring amino acid, preferably no cysteine.

In the polypeptides described above, the one or more immunoglobulin single variable domains and the one or more groups, residues, moieties or binding units may be linked directly to each other and/or via one or more suitable linkers or spacers. For example, when the one or more groups, residues, moieties or binding units are amino acids, the linkers may also be an amino acid, so that the resulting polypeptide is a fusion protein or fusion polypeptide.

As used herein, the term “linker” denotes a peptide that fuses together two or more ISVs into a single molecule. The use of linkers to connect two or more (poly) peptides is well known in the art. Further exemplary peptidic linkers are shown in Table B. One often used class of peptidic linker are known as the “Gly-Ser” or “GS” linkers. These are linkers that essentially consist of glycine (G) and serine(S) residues, and usually comprise one or more repeats of a peptide motif such as the GGGGS (SEQ ID NO: 154) motif (for example, having the formula (Gly-Gly-Gly-Gly-Ser) n (SEQ ID NO: 152) in which n may be 1, 2, 3, 4, 5, 6, 7 or more). Some often-used examples of such GS linkers are 9GS linkers (GGGGSGGGS, SEQ ID NO: 157), 15GS linkers (n=3) and 3SGS linkers (n=7). Reference is for example made to Chen et al. 2013 (Adv. Drug Deliv. Rev. 65 (10): 1357-1369) and Klein et al. 2014 (Protein Eng. Des. Sel. 27 (10): 325-330).

TABLE B
Linker sequences (“ID” refers to the SEQ ID NO as used herein)

| Name | | Amino acid sequence |
|---|---|---|
| 3A linker | ID | AAA |
| 5GS linker | 154 | GGGGS |
| 7GS linker | 155 | SGGSGGS |
| 8GS linker | 156 | GGGGSGGS |
| 9GS linker | 157 | GGGGSGGGS |
| 10GS linker | 158 | GGGGSGGGGS |
| 15GS linker | 159 | GGGGGGGGSGGGGS |
| 18GS linker | 160 | GGGGSGGGGSGGGGSGGS |
| 20GS linker | 161 | GGGGSGGGGSGGGGSGGGGS |
| 25GS linker | 162 | GGGGSGGGGSGGGGSGGGGSGGGGS |
| 30GS linker | 163 | GGGGSGGGGSGGGGSGGGGSGGGGSGGGGS |
| 35GS linker | 164 | GGGGSGGGGSGGGGSGGGGSGGGGSGGGGSGGGGS |
| 40GS linker | 165 | GGGGSGGGGSGGGGSGGGGGGGGSGGGGSGGGGSGGGGS |
| Gl hinge | 166 | EPKSCDKTHTCPPCP |
| 9GS-G1 hinge | 167 | GGGGSGGGSEPKSCDKTHTCPPCP |
| Llama upper long | 168 | EPKTPKPQPAAA |
| hinge region | | |
| G3 hinge | 169 | ELKTPLGDTTHTCPRCPEPKSCDTPPPCPRCPEPKSCDTPPPCP |
| | | RCPEPKSCDTPPPCPRCP |

In one aspect, the disclosure also relates to such amino acid sequences and/or Nanobodies that can bind to and/or are directed against CD8 and that comprise CDR sequences that are generally as further defined herein, to suitable fragments thereof, as well as to polypeptides that comprise or essentially consist of one or more of such Nanobodies and/or suitable fragments. In some aspect, the disclosure relates to Nanobodies with SEQ ID NO: 77. In particular, the disclosure in some specific aspects provides:
I) amino acid sequences that are directed against CD8 and that have at least 80%, preferably at least 85%, such as 90% or 95% or more sequence identity with SEQ ID NO: 77;
II) amino acid sequences that cross-block the binding of the amino acid sequence of SEQ ID NO: 77 to CD8 and/or that compete with at least the amino acid sequence of SEQ ID NO: 77 for binding to CD8;

Such amino acid sequences may be as further described herein (and may for example be Nanobodies); as well as polypeptides of the disclosure that comprise one or more of such amino acid sequences (which may be as further described herein), and particularly bispecific (or multispecific) polypeptides as described herein, and nucleic acid sequences that encode such amino acid sequences and polypeptides. Such amino acid sequences and polypeptides do not include any naturally occurring ligands.

In some embodiments, the CD8 is derived from a mammalian animal, such as a human being. In one specific, but non-limiting aspect, the disclosure relates to an amino acid sequence directed against CD8, that comprises:
a) the amino acid sequence of SEQ ID NO: 77;
b) amino acid sequences that have at least 80% amino acid identity with a SEQ ID NO: 77, or
c) amino acid sequences that have 3, 2, or 1 amino acid difference with SEQ ID NO: 77;
   or any suitable combination thereof.

In some embodiments, disclosed is a Nanobody against CD8, which consist of 4 framework regions (FR1 to FR4 respectively) and 3 complementarity determining regions (CDR1 to CDR3 respectively). In some embodiments, in such a Nanobody:
(I) CDR1 comprises or essentially consists of an amino acid sequence of GSTFSDYG (SEQ ID NO: 100),
or amino acid sequences that have at least 80%, at least 90%, at least 95%, at least 99% or more sequence identity with GSTFSDYG (SEQ ID NO: 100), in which (1) any amino acid substitution is a conservative amino acid substitution; and/or (2) said amino acid sequence only comprises amino acids substitutions, and no amino acid deletions or insertions, compared to GSTFSDYG (SEQ ID NO: 100);
and/or from the group consisting of amino acids sequences that have 2 or only 1 amino acid difference(s) with GSTFSDYG (SEQ ID NO: 100), in which
any amino acid substitution is a conservative amino acid substitution; and/or
said amino acid sequence only comprises amino acid substitutions, and no amino acid deletions or insertions, compared to GSTFSDYG (SEQ ID NO: 100).
(II) CDR2 comprises or essentially consists of an amino acid sequence of IDWNGEHT (SEQ ID NO: 101),
or amino acid sequences that have at least 80%, at least 90%, at least 95%, at least 99% or more sequence identity with IDWNGEHT (SEQ ID NO: 101), in which (1) any amino acid substitution is a conservative amino acid substitution; and/or (2) said amino acid sequence only comprises amino acids substitutions, and no amino acid deletions or insertions, compared to IDWNGEHT (SEQ ID NO: 101);
and/or from the group consisting of amino acids sequences that have 2 or only 1 amino acid difference(s) with IDWNGEHT (SEQ ID NO: 101), in which
any amino acid substitution is a conservative amino acid substitution; and/or
said amino acid sequence only comprises amino acid substitutions, and no amino acid deletions or insertions, compared to IDWNGEHT (SEQ ID NO: 101).
(III) CDR3 comprises or essentially consists of an amino acid sequence of AADALPYTVRKYNY (SEQ ID NO: 102),
or amino acid sequences that have at least 80%, at least 90%, at least 95%, at least 99% or more sequence identity with AADALPYTVRKYNY (SEQ ID NO: 102), in which (1) any amino acid substitution is a conservative amino acid substitution; and/or (2) said amino acid sequence only comprises amino acids substitutions, and no amino acid deletions or insertions, compared to AADALPYTVRKYNY (SEQ ID NO: 102);
and/or from the group consisting of amino acids sequences that have 2 or only 1 amino acid difference(s) with AADALPYTVRKYNY (SEQ ID NO: 102), in which
any amino acid substitution is a conservative amino acid substitution; and/or
said amino acid sequence only comprises amino acid substitutions, and no amino acid deletions or insertions, compared to AADALPYTVRKYNY (SEQ ID NO: 102).

CD8 Nanobodies as disclosed herein may comprise one, two or all three of the CDRs explicitly listed above. In some embodiments, the CD8 Nanobody comprises:

CDR1:
(SEQ ID NO: 100)
GSTFSDYG,
based on IMGT designation;
CDR2:
(SEQ ID NO: 101)
IDWNGEHT,
based on IMGT designation;
and
CDR3:
(SEQ ID NO: 102)
AADALPYTVRKYNY,
based on IMGT designation.

In the Nanobodies of the disclosure that comprise the combinations of CDRs mentioned above, each CDR can be replaced by a CDR chosen from the group consisting of amino acid sequences that have at least 80%, preferably at least 90%, more preferably at least 95%, even more preferably at least 99% sequence identity with the mentioned CDRs; in which:
(1) any amino acid substitution is preferably a conservative amino acid substitution; and/or
(2) said amino acid sequence preferably only comprises amino acid substitutions, and no amino acid deletions or insertions, compared to the above amino acid sequence(s);
and/or chosen from the group consisting of amino acid sequences that have 3, 2 or only 1 (as indicated in the preceding paragraph) “amino acid difference(s)” with the mentioned CDR(s) one of the above amino acid sequences, in which:
(1) any amino acid substitution is preferably a conservative amino acid substitution; and/or
(2) said amino acid sequence preferably only comprises amino acid substitutions, and no amino acid deletions or insertions, compared to the above amino acid sequence(s).

In one embodiment, the CD8 Nanobody is BDSn:

(SEQ ID NO: 77)
Anti-CD8 BDSn Nb sequence (CDR1, CDR2, CDR3
underlined based on IMGT designation):
EVQLVESGGGLVQAGGSLRLSCAASGSTFSDYGVGWFRQAPG
KGREFVADIDWNGEHTSYADSVKGRFATSRDNAKNTAYLQMN
SLKPEDTAVYYCAADALPYTVRKYNYWGQGTQVTVSSGGCGG
HHHHHH

In some embodiments, a CD8 Nanobody of the present disclosure binds to CD8 with a dissociation constant (KD) of 10⁻⁵ to 10⁻¹² moles/liter (M) or less, and preferably 10⁻⁷ to 10⁻¹² moles/liter (M) or less and more preferably 10⁻⁸ to 10⁻¹² moles/liter (M), and/or with an association constant (KA) of at least 107 M−1, preferably at least 10⁸ M⁻¹, more preferably at least 10⁹ M⁻¹, such as at least 10¹² M⁻¹; and in particular with a KD less than 500 nM, preferably less than 200 nM, more preferably less than 10 nM, such as less than 5 nM. The KD and KA values of the Nanobody of the disclosure against vWF can be determined in a manner known per se, for example using the assay described herein. More generally, the Nanobodies described herein preferably have a dissociation constant with respect to vWF that is as described in this paragraph.

Generally, it should be noted that the term Nanobody as used herein in its broadest sense is not limited to a specific biological source or to a specific method of preparation. For example, as will be discussed in more detail below, the Nanobodies can be obtained (1) by isolating the VHH domain of a naturally occurring heavy chain antibody; (2) by expression of a nucleotide sequence encoding a naturally occurring VHH domain; (3) by “humanization” (as described below) of a naturally occurring VHH domain or by expression of a nucleic acid encoding a such humanized VHH domain; (4) by “camelization” (as described below) of a naturally occurring VH domain from any animal species, in particular a species of mammal, such as from a human being, or by expression of a nucleic acid encoding such a camelized VH domain; (5) by “camelisation” of a “domain antibody” or “Dab” as described by Ward et al (supra), or by expression of a nucleic acid encoding such a camelized VH domain; (6) using synthetic or semi-synthetic techniques for preparing proteins, polypeptides or other amino acid sequences; (7) by preparing a nucleic acid encoding a Nanobody using techniques for nucleic acid synthesis, followed by expression of the nucleic acid thus obtained; and/or (8) by any combination of the foregoing. Suitable methods and techniques for performing the foregoing will be clear to the skilled person based on the disclosure herein and for example include the methods and techniques described in more detail hereinbelow.

In some embodiments, the CD8 Nanobodies of the present disclosure do not have an amino acid sequence that is exactly the same as (i.e. as a degree of sequence identity of 100% with) the amino acid sequence of a naturally occurring VH domain, such as the amino acid sequence of a naturally occurring VH domain from a mammal, and in particular from a human being.

One class of CD8 Nanobodies of the disclosure comprises Nanobodies with an amino acid sequence that corresponds to the amino acid sequence of a naturally occurring VHH domain, but that has been “humanized”, i.e. by replacing one or more amino acid residues in the amino acid sequence of said naturally occurring VHH sequence by one or more of the amino acid residues that occur at the corresponding position(s) in a VH domain from a conventional 4-chain antibody from a human being (e.g., indicated above). It should be noted that such humanized CD8 Nanobodies of the present disclosure can be obtained in any suitable manner known per se (i.e. as indicated under points (1)-(8) above) and thus are not strictly limited to polypeptides that have been obtained using a polypeptide that comprises a naturally occurring VHH domain as a starting material.

Another class of CD8 Nanobodies of the present disclosure comprises Nanobodies with an amino acid sequence that corresponds to the amino acid sequence of a naturally occurring VH domain that has been “camelized”, i.e. by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. This can be performed in a manner known per se, which will be clear to the skilled person, for example on the basis of the further description below. Reference is also made to WO 94/04678. Such camelization may preferentially occur at amino acid positions which are present at the VH-VL interface and at the so-called Camelidae hallmark residues (see for example also WO 94/04678), as also mentioned below. In some embodiments, the VH domain or sequence that is used as a starting material or starting point for generating or designing the camelized Nanobody is a VH sequence from a mammal, e.g., VH sequence of a human being. It should be noted that such camelized Nanobodies of the present disclosure can be obtained in any suitable manner known per se and thus are not strictly limited to polypeptides that have been obtained using a polypeptide that comprises a naturally occurring VH domain as a starting material.

For example, both “humanization” and “camelization” can be performed by providing a nucleotide sequence that encodes such a naturally occurring VHH domain or VH domain, respectively, and then changing, in a manner known per se, one or more codons in said nucleotide sequence such that the new nucleotide sequence encodes a humanized or camelized Nanobody of the present disclosure, respectively, and then expressing the nucleotide sequence thus obtained in a manner known per se so as to provide the desired Nanobody. Alternatively, based on the amino acid sequence of a naturally occurring VHH domain or VH domain, respectively, the amino acid sequence of the desired humanized or camelized Nanobody of the present disclosure, respectively, can be designed and then synthesized de novo using techniques for peptide synthesis known per se. Also, based on the amino acid sequence or nucleotide sequence of a naturally occurring VHH domain or VH domain, respectively, a nucleotide sequence encoding the desired humanized or camelized Nanobody can be designed and then synthesized de novo using techniques for nucleic acid synthesis known per se, after which the nucleotide sequence thus obtained can be expressed in a manner known per se so as to provide the desired Nanobody.

Other suitable ways and techniques for obtaining Nanobodies and/or nucleotide sequences and/or nucleic acids encoding the same, starting from (the amino acid sequence of) naturally occurring VH domains or preferably VHH domains and/or from nucleotide sequences and/or nucleic acid sequences encoding the same will be clear from the skilled person, and may for example comprising combining one or more amino acid sequences and/or nucleotide sequences from naturally occurring VH domains (such as one or more FR's and/or CDR's) with one or more one or more amino acid sequences and/or nucleotide sequences from naturally occurring VHH domains (such an one or more FR's or CDR's), in a suitable manner so as to provide (a nucleotide sequence or nucleic acid encoding) a Nanobody. Also provided are compounds and constructs, and in particular proteins and polypeptides that comprise or essentially consists of at least one such amino acid sequence and/or Nanobody of the disclosure (or suitable fragments thereof), and optionally further comprises one or more other groups, residues, moieties or binding units. In some embodiments, such further groups, residues, moieties, binding units or amino acid sequences may or may not provide further functionality to the amino acid sequence and/or Nanobody (and/or to the compound or construct in which it is present) and may or may not modify the properties of the amino acid sequence and/or Nanobody.

The disclosure also encompasses any polypeptide of the present disclosure that has been glycosylated at one or more amino acid positions, usually depending on the host used to express the polypeptide. a polypeptide can comprise an amino acid sequence of a CD8 Nanobody of the present disclosure, which is fused at its amino terminal end, at its carboxy terminal end, or both at its amino terminal end and at its carboxy terminal end with at least one further amino acid sequence. Such further amino acid sequence may comprise at least one further Nanobody, so as to provide a polypeptide that comprises at least two, such as three, four or five, Nanobodies, in which said Nanobodies may optionally be linked via one or more linker sequences (as defined herein). Polypeptides of comprising CD8 Nanobody of the present disclosure and one or more another Nanobodies are multivalent polypeptides. In a multivalent polypeptide, the two or more Nanobodies may be the same or different. For example, the two or more Nanobodies in a multivalent polypeptide:
may be directed against the same antigen, i.e. against the same parts or epitopes of said antigen or against two or more different parts or epitopes of said antigen; and/or:
may be directed against the different antigens;
or a combination thereof.
   Thus, a bivalent polypeptide, for example:
may comprise two identical Nanobodies;
may comprise a first Nanobody directed against a first part or epitope of an antigen and a second Nanobody directed against the same part or epitope of said antigen or against another part or epitope of said antigen;
   or may comprise a first Nanobody directed against a first antigen and a second Nanobody directed against a second antigen different from said first antigen;
   whereas a trivalent Polypeptide of the Invention for example:
may comprise three identical or different Nanobodies directed against the same or different parts or epitopes of the same antigen;
may comprise two identical or different Nanobodies directed against the same or different parts or epitopes on a first antigen and a third Nanobody directed against a second antigen different from said first antigen; or
may comprise a first Nanobody directed against a first antigen, a second Nanobody directed against a second antigen different from said first antigen, and a third Nanobody directed against a third antigen different from said first and second antigen.

The CD8 Nanobodies and polypeptides as disclosed herein can also be introduced and expressed in one or more cells, tissues or organs of a multicellular organism, for example for prophylactic and/or therapeutic purposes (e.g., as a gene therapy). For this purpose, the nucleotide sequences encoding the CD8 Nanobodies or polypeptides as disclosed herein can be introduced into the cells or tissues in any suitable way, for example as such (e.g., using liposomes) or after they have been inserted into a suitable gene therapy vector (for example derived from retroviruses such as adenovirus, or parvoviruses such as adeno-associated virus). As will also be clear to the skilled person, such gene therapy may be performed in vivo and/or in situ in the body of a patent by administering a nucleic acid of the invention or a suitable gene therapy vector encoding the same to the patient or to specific cells or a specific tissue or organ of the patient; or suitable cells (often taken from the body of the patient to be treated, such as explanted lymphocytes, bone marrow aspirates or tissue biopsies) may be treated in vitro with a nucleotide sequence of the invention and then be suitably (re-)introduced into the body of the patient. All this can be performed using gene therapy vectors, techniques and delivery systems which are well known to the skilled person, for Culver, K. W., “Gene Therapy”, 1994, p. xii, Mary Ann Liebert, Inc., Publishers, New York, N.Y.). Giordano, Nature F Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91; (1998), 30-36; Verma, Gene Ther. 5 (1998), 692-699; Nabel, Ann. N.Y. Acad. Sci.: 811 (1997), 289-292; Verzeletti, Hum. Gene Ther. 9 (1998), 2243-51; Wang, Nature Medicine 2 (1996), 714-716; WO 94/29469; WO 97/00957, U.S. Pat. No. 5,580,859; 1 U.S. Pat. No. 5,589,5466; or Schaper, Current Opinion in Biotechnology 7 (1996), 635-640. For example, in situ expression of ScFv fragments (Afanasieva et al., Gene Ther., 10, 1850-1859 (2003)) and of diabodies (Blanco et al., J. Immunol, 171, 1070-1077 (2003)) has been described in the art.

Accordingly, nucleic acid sequences encoding the CD8 Nanobodies as described herein, and expression construct and host cells comprising the nucleic acid sequence are also provided.

Also disclosed are methods of using CD8 Nanobodies and polypeptides of the present disclosure.

In some embodiments, a polypeptide comprising a CD8 Nanobody can be used in the lipid nanoparticles of the present disclosure for delivering a nucleic acid into an immune cell, as described herein. In some embodiments, CD8 Nanobodies and polypeptides of the present disclosure can be used to treat a condition or a disease in a subject in need thereof. In some embodiments, such conditions or diseases include, but are not limited to, cancer, infections, immune disorders, autoimmune diseases.

In some embodiments, a polypeptide comprising a CD8 Nanobody can be used in an imaging agent. In some embodiments, the imaging agent allows for the detection of human CD8 which is a specific biomarker found on the surface of a subset of T-cell for diagnostic imaging of the immune system. Imaging of CD8 allows for the in vivo detection of T-cell localization. Changes in T-cell localization can reflect the progression of an immune response and can occur over time as a result of various therapeutic treatments or even disease states. In some embodiments, it is used for imaging T-cell localization for immunotherapy.

In addition, CD8 plays a role in activating downstream signaling pathways that are important for the activation of cytolytic T cells that function to clear viral pathogens and provide immunity to tumors. CD8 positive T cells can recognize short peptides presented within the MHCl protein of antigen presenting cells. In some embodiments, a polypeptide comprising a CD8 Nanobody can potentiate signaling through the T cell receptor and enhance the ability of a subject to clear viral pathogens and respond to tumor antigens. Thus, in some embodiments, the antigen binding constructs provided herein can be agonists and can activate the CD8 target.

### II. Ionizable Cationic Lipids

Provided herein are ionizable cationic lipids that can be used to produce lipid nanoparticle compositions to facilitate the delivery of a payload (e.g., a nucleic acid, such as a DNA or RNA, such as an mRNA) encapsulated therein to cells, e.g., mammalian cells, e.g., human cells, e.g., immune cells or hematopoietic cells. The ionizable cationic lipids have been designed to enable intracellular delivery of a nucleic acid, e.g., mRNA, to the cytosolic compartment of a target cell type and rapidly degrade into non-toxic components. The complex functionalities of the ionizable cationic lipids are facilitated by the interplay between the chemistry and geometry of the ionizable lipid head group, the hydrophobic “acyl-tail” groups and the linkers connecting the head group and the acyl tail groups. Typically, the pKₐ of the ionizable amine head group is designed to be in the range of 6-8, such as between 6.2-7.4, or between 6.7-7.2, such that it remains strongly cationic under acidic formulation conditions (e.g., pH 4-pH 5.5), neutral or slightly anionic in physiological pH (7.4) and cationic in the early and late endosomal compartments (e.g., pH 5.5-pH 7). The acyl-tail groups play a key role in fusion of the lipid nanoparticle with endosomal membranes and membrane destabilization through structural perturbation. The three-dimensional structure of the acyl-tail (determined by its length, and degree and site of unsaturation) along with the relative sizes of the head group and tail group are thought to play a role in promoting membrane fusion, and hence lipid nanoparticle endosomal escape (a key requirement for cytosolic delivery of a nucleic acid payload). The linker connecting the head group and acyl tail groups is designed to degrade by physiologically prevalent enzymes (e.g., esterases, or proteases) or by acid catalyzed hydrolysis.

In one aspect, provided is a compound of Formula (I):

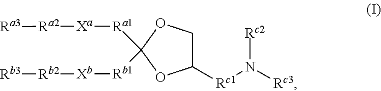

or a salt thereof, wherein:
R^{a1} and R^{b1} are each independently C₁₋₁₂ alkylene;
X^{a} and X^{b} are each independently —C(O)O—* or —OC(O)—*, wherein * indicates the point of attachment to R^{a1} or R^{b1};
R^{a2} and R^{b2} are each independently a bond or C₁₋₃ alkylene;
R^{a3} is

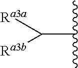

and R^{b3} is

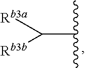

wherein R^{a3a}, R^{a3b}, R^{b3a}, and R^{b3b} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3;
R^{c1} is C₁₋₆ alkylene;
R^{c2} is H or C₁₋₆ alkyl; and
R^{c3} is C₁₋₆ alkyl,

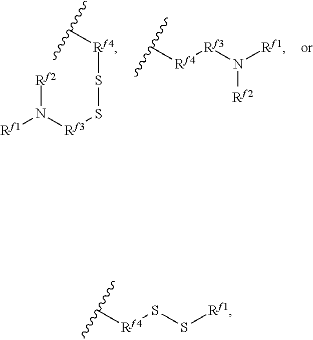

wherein:
   R^{f1} is H, C₁₋₆ alkyl, or

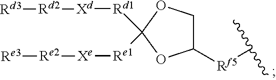

R^{f2} is H, C₁₋₆ alkyl, or —C(O)O—C₂₋₆ alkenyl;
R^{f3}, R^{f4}, and R^{f5} are each independently C₁₋₆ alkylene; and
R^{d1} and R^{e1} are each independently C₁₋₁₂ alkylene;
X^{d} and X^{e} are each independently —C(O)O—* or —OC(O)—*, wherein * indicates the point of attachment to R^{d1} or R^{e1};
R^{d2} and R^{e2} are each independently a bond or C₁₋₃ alkylene; and
R^{d3} is

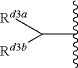

and R^{c3} is

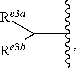

wherein R^{d3a}, R^{d3b}, R^{c3a}, and R^{c3b} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3;
with the proviso that when R^{c1} is —CH₂— and R^{c2} and R^{c3} are each methyl, then none of R^{a3a}, R^{a3b}, R^{b3a}, and R^{b3b} is H; when Rol is —(CH₂)₂— and R^{c2} and R^{c3} are each methyl, then R^{b3a} is not H and R^{b3b} is ethyl; when R^{c1} is —(CH₂)₂— and R^{c2}, R^{c3}, or both R^{c2} and R^{c3} are not methyl, then none of R^{a3a}, R^{a3b}, R^{b3a}, and R^{b3b} is H; when R^{c1} is —(CH₂)₃—, R^{c2} and R^{c3} are each methyl, and one of R^{a3a}, R^{a3b}, R^{b3a}, and R^{b3b} is H, then at least one of R^{a3a}, R^{a35}, R^{b3a}, and R^{b3b} that is not His substituted with a heterocylyl; and when R^{c1} is —(CH₂)₄—, R^{c2} and R^{c3} are each methyl, then none of R^{a3a}, R^{a3b}, R^{b3a}, and R^{b3b} is H.

In one aspect, provided is a compound of Formula (I-P2):

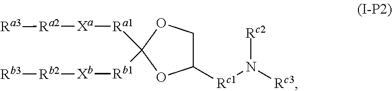

or a salt thereof, wherein:
R^{a1} and R^{b1} are each independently C₁₋₁₂ alkylene;
X^{a} and X^{b} are each independently —C(O)O—* or —OC(O)—*, wherein * indicates the point of attachment to R^{a1} or R^{b1};
R^{a2} and R^{b2} are each independently a bond or C₁₋₃ alkylene;
R^{a3} is

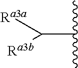

and R^{b3} is

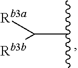

wherein R^{a3a}, R^{a3b}, R^{b3a}, and R^{b3b} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3;
R^{c1} is C₁₋₆ alkylene;
R^{c2} is H or C₁₋₆ alkyl; and
R^{c3} is C₁₋₆ alkyl or

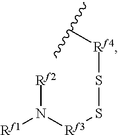

wherein:
   R^{f1} is H, C₁₋₆ alkyl, or

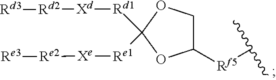

R^{f2} is H, C₁₋₆ alkyl, or —C(O)O—C₂₋₆ alkenyl;
R^{f3} and R^{f4} are each independently C₁₋₆ alkylene; and
R^{d1} and R^{e1} are each independently C₁₋₁₂ alkylene;
x^{d} and X^{e} are each independently —C(O)O—* or —OC(O)—*, wherein * indicates the point of attachment to R^{d1} or R^{e1},
R^{d2} and R^{e2} are each independently a bond or C₁₋₃ alkylene; and
R^{d3} is

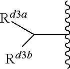

and R^{c3} is

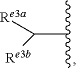

wherein R^{d3a}, R^{d3b}, R^{c3a}, and R^{c3b} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3;
   with the proviso that when Rol is —CH₂— and R^{c2} and R^{c3} are each methyl, then none of R^{a3a}, R^{a3b} R^{b3a}, and R^{b3b} is H; when R^{c1} is —(CH₂)₂— and R^{c2} and R^{c3} are each methyl, then R^{b3a} is not H and R^{b3b} is ethyl; when R^{c1} is —(CH₂)₂— and R^{c2}, R^{c3}, or both R^{c2} and R^{c3} are not methyl, then none of R^{a3a}, R^{a3b}, R^{b3a}, and R^{b3b} is H; when R^{c1} is —(CH₂)₃—, R^{c2} and R^{c3} are each methyl, and one of R^{a3a}, R^{a3b}, R^{b3a}, and R^{b3b} is H, then at least one of R^{a3a}, R^{a3b}, R^{b3a}, and R^{b3b} that is not His substituted with a heterocylyl; and when R^{c1} is —(CH₂)₄—, R^{c2} and R^{c3} are each methyl, then none of R^{a3a}, R^{a3b}, R^{b3a}, and R^{b3b} is H.

In one aspect, provided is a compound of Formula (I-P1);

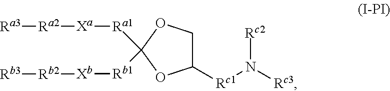

or a salt thereof, wherein:
R^{a1} and R^{b1} are each independently C₁₋₁₂ alkylene;
X^{a} and X^{b} are each independently —C(O)O—* or —OC(O)—*, wherein * indicates the point of attachment to R^{a1} or R^{b1};
R^{a2} and R^{b2} are each independently a bond or C₁₋₃ alkylene;
R^{a3} is

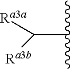

and R^{b3} is

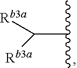

wherein R^{a3a}, R^{a3b}, R^{b3a}, and R^{b3b} are each independently H or C₁₋₁₂ alkyl optionally substituted with heterocylyl;
R^{c1} is C₁₋₆ alkylene;
R^{c2} is C₁₋₆ alkyl; and
R^{c3} is C₁₋₆ alkyl or

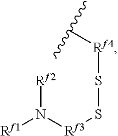

wherein:
   R^{f1} is H or

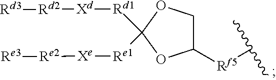

R^{f2} is C₁₋₆ alkyl;
R^{f3} and R^{f4} are each independently C₁₋₆ alkylene; and
R^{d1} and R^{e1} are each independently C₁₋₁₂ alkylene;
X^{d} and X^{e} are each independently —C(O)O—* or —OC(O)—*, wherein * indicates the point of attachment to R^{d1} or R^{e1},
R^{d2} and R^{e2} are each independently a bond or C₁₋₃ alkylene; and
R^{d3} is

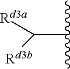

and R^{E3} is

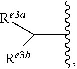

wherein R^{d3a}, R^{d3b}, R^{e3a}, and R^{e3b} are each independently H or C₁₋₁₂ alkyl optionally substituted with heterocylyl;
with the proviso that when R^{c1} is —CH₂— and R^{c2} and R^{c3} are each methyl, then none of R^{a3a}, R^{a3b}, R^{b3a}, and R^{b3b} is H; when R^{c1} is —(CH₂)₂— and R^{c2} and R^{c3} are each methyl, then R^{b3a} is not H and R^{b3b} is ethyl; when R^{c1} is —(CH₂)₂— and R^{c2}, R^{c3}, or both R^{c2} and R^{c3} are not methyl, then none of R^{a3a}, R^{a3b}, R^{b3a}, and R^{b3b} is H; when R^{c1} is —(CH₂)₃—, R^{c2} and R^{c3} are each methyl, and one of R^{a3a}, R^{a3b}, R^{b3a}, and R^{b3b} is H, then at least one of R^{a3a}, R^{a3b}, R^{b3a}, and R^{b3b} that is not H is substituted with a heterocylyl; and when R^{c1} is —(CH₂)₄—, R^{c2} and R^{c3} are each methyl, then none of R^{a3a}, Rabb, R^{b3a}, and R^{b3b} is H.

In some embodiments, R^{a1} and R^{b1} are each independently a linear alkylene. In some embodiments, R^{a1} and R^{b1} are each independently C₅₋₁₀ alkylene. In some embodiments, R^{a1} and R^{b1} are each —(CH₂)₇—.

In some embodiments, X^{a} and X^{b} are each —C(O)O—*. In some embodiments, X^{a} and X^{b} are each —OC(O)—*.

In some embodiments, R^{a2} is C₁₋₃ alkylene. In some embodiments, R^{b2} is C₁₋₃ alkylene. In some embodiments, R^{a2} is a bond. In some embodiments, R^{b2} is a bond. In some embodiments, R^{a2} and R^{b2} are each a bond. In some embodiments, R^{a2} and R^{b2} are each —CH₂—.

In some embodiments, no more than one of R^{a3a}, R^{a3b}, R^{b3a}, and R^{b3b} is H. In some embodiments, R^{a3a}, R^{a3b}, R^{b3a}, and R^{b3b} are each independently a linear alkyl. In some embodiments, R^{a3a}, R^{a3b}, R^{b3a}, and R^{b3b} are each independently C₂₋₁₀ alkyl. In some embodiments, R^{a3a}, R^{a3b}, R^{b3a}, and R^{b3b} are each independently C₂₋₈ alkyl. In some embodiments, R^{a3a}, R^{a3b}, R^{b3a}, and R^{b3b} are each independently H or optionally substituted with a heterocyclyl comprising a disulfide bond. In some embodiments, R^{a3a}, R^{a3b}, R^{b3a}, and R^{b3b} are each independently H or optionally substituted with 1,2-dithiolanyl. In some embodiments, none of R^{a3b}, Rabb, R^{b3b}, and R^{b3b} is H. In some embodiments, at least one of R^{a3a}, R^{a3b}, R^{b3a}, and R^{b3b} is substituted with a heterocyclyl. In some embodiments, at least one of R^{a3a}, R^{a3b}, R^{b3a}, and R^{b3b} is C₁₋₁₂ alkyl substituted with a 5- to 10-membered heterocyclyl comprising a disulfide bond or —(C₁₋₁₀ alkylene)-Sₙ—(C₁₋₁₀ alkyl). In some embodiments, at least one of R^{a3a}, R^{a3b}, R^{b3a}, and R^{b3b} is C₁₋₁₂ alkyl substituted with a 5- to 10-membered heterocyclyl comprising a disulfide bond. In some embodiments, at least one of R^{a3a}, R^{a3b}, R^{b3a}, and R^{b3b} is —(C₁₋₁₀ alkylene)-Sₙ—(C₁₋₁₀ alkyl). In some embodiments, the heterocyclyl comprising a disulfide bond is 1,2-dithiolanyl.

In some embodiments, R^{a3} and R^{b3} are each independently

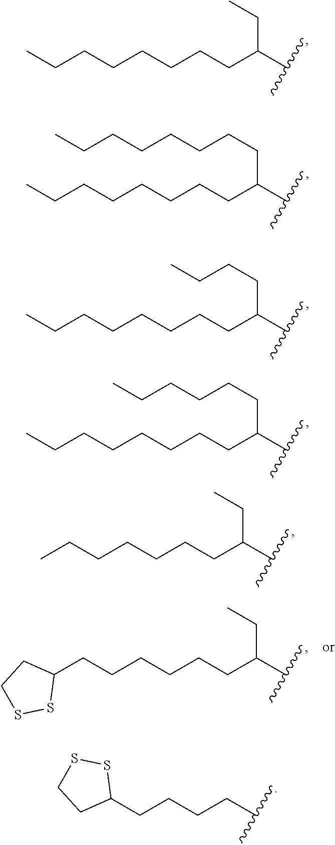

In some embodiments, R^{a3} and R^{b3} are each independently

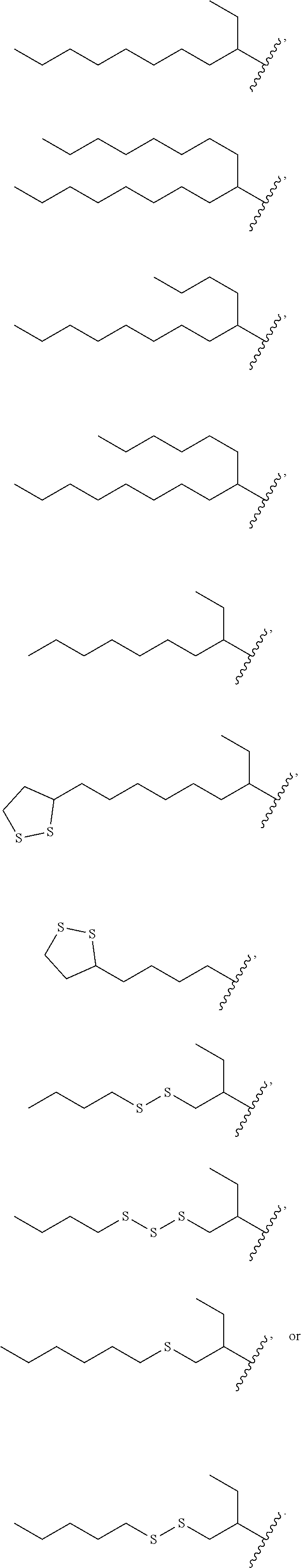

In some embodiments, R^{a3} and R^{b3} are each independently

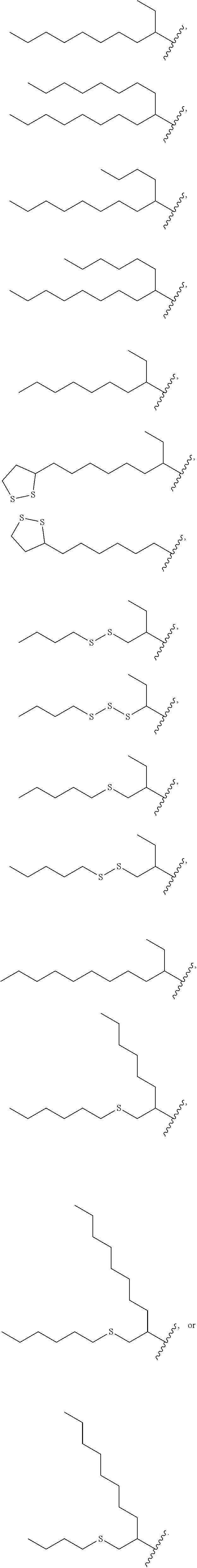

In some embodiments, R^{a3} and R^{b3} are the same.

In some embodiments, R^{c1} is —(CH₂)₂—. In some embodiments, R^{c1} is —(CH₂)₃—. In some embodiments, R^{c1} is —(CH₂)₄—.

In some embodiments, R^{c2} is methyl. In some embodiments, R^{c2} is ethyl.

In some embodiments, R^{c3} is C₁₋₆ alkyl. In some embodiments, R^{c3} is methyl. In some embodiments, R^{c3} is ethyl.

In some embodiments, when R^{c1} is —(CH₂)₂— and R^{c2} is methyl, then R^{c3} is not methyl. In some embodiments, when R^{c2} is methyl and Ref is —(CH₂)₂—, then R^{c3} is not methyl.

In some embodiments, R^{c3} is

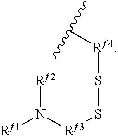

In some embodiments, R^{c3} is

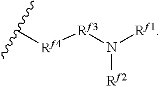

In some embodiments, R^{c3} is

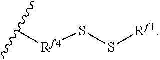

In some embodiments, R^{f1} is H. In some embodiments, R^{f1} is methyl. In some embodiments, R^{f1} is n-butyl. In some embodiments, R^{f1} is C₁₋₆ alkyl.

In some embodiments, R^{f1} is

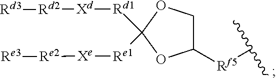

In some embodiments, R^{f2} is H. In some embodiments, R^{e2} is methyl. In some embodiments, R^{f2} is ethyl. In some embodiments, R^{f2} is C₁₋₆ alkyl. In some embodiments, R^{f2} is —C(O)O—C₂₋₆ alkenyl. In some embodiments, R^{f2} is —C(O)O—CH₂CH═CH₂.

In some embodiments, R^{f3} and R^{f4} are each —(CH₂)₂—. In some embodiments, R^{f3} and R^{f4} are each —(CH₂)₃—.

In some embodiments, R^{f4} is —(CH₂)₂—.

In some embodiments, R^{f5} is —(CH₂)₂—. In some embodiments, R^{f5} is —(CH₂)₃—. In some embodiments, R^{f5} is —(CH₂)₄—.

In some embodiments, R^{d1} and R^{e1} are each independently a linear alkyelene. In some embodiments, R^{d1} and R^{E1} are each independently C₅₋₁₀ alkylene. In some embodiments, R^{d1} and R^{E1} are each —(CH₂) 7˜

In some embodiments, X^{d} and X^{e} are each —C(O)O—*. In some embodiments, X^{d} and X^{e} are each —OC(O)—*

In some embodiments, R^{d2} is C₁₋₃ alkylene. In some embodiments, R^{c2} is C₁₋₃ alkylene. In some embodiments, R^{a2} is a bond. In some embodiments, R^{c2} is a bond. In some embodiments, R^{d2} and R^{e2} are each a bond. In some embodiments, R^{d2} and R^{e2} are each —CH₂—.

In some embodiments, no more than one of R^{d3a}, R^{d3b}, R^{c3a}, and R^{c3b} is H. In some embodiments, R^{d3a}, R^{d3b}, R^{c3a}, and R^{c3b} are each independently a linear alkyl. In some embodiments, R^{d3a}, R^{d3b}, R^{c3a}, and R^{c3b} are each independently C₂₋₁₀ alkyl. In some embodiments, R^{D3a}, R^{d3b}, R^{c3a}, and R^{c3b} are each independently C₂₋₈ alkyl. In some embodiments, R^{d3a}, R^{d3b}, R^{c3a} and R^{c3b} are each independently H or optionally substituted with a heterocyclyl comprising a disulfide bond. In some embodiments, R^{d3a}, R^{d3b}, R^{c3a}, and R^{c3b} are each independently H or optionally substituted with 1,2-dithiolanyl. In some embodiments, none of R^{d3a}, R^{d3b}, R^{c3a}, and R^{c3b} is H. In some embodiments, at least one of R^{d3a}, R^{d3b}, R^{c3a}, and R^{c3b} is substituted with a heterocyclyl. In some embodiments, at least one of R^{d3a}, R^{d3b}, R^{c3a}, and R^{c3b} is C₁₋₁₂ alkyl substituted with a 5- to 10-membered heterocyclyl comprising a disulfide bond or —(C₁₋₁₀ alkylene)-Sₙ—(C₁₋₁₀ alkyl). In some embodiments, at least one of R^{d3a}, R^{d3b}, R^{c3a}, and R^{c3b} is C₁₋₁₂ alkyl substituted with a 5- to 10-membered heterocyclyl comprising a disulfide bond. In some embodiments, at least one of R^{d3}a, R^{d3b}, R^{c3a}, and R^{c3b} is —(C₁₋₁₀ alkylene)-Sₙ—(C₁₋₁₀ alkyl). In some embodiments, the heterocyclyl comprising a disulfide bond is 1,2-dithiolanyl.

In some embodiments, R^{d3} and R^{e3} are each independently

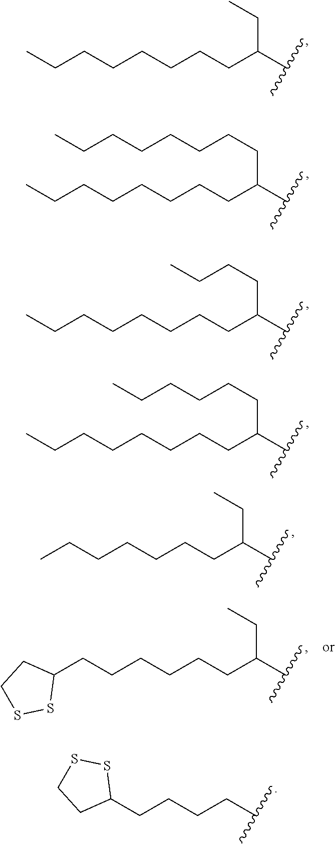

In some embodiments, R^{d3} and R^{e3} are each independently

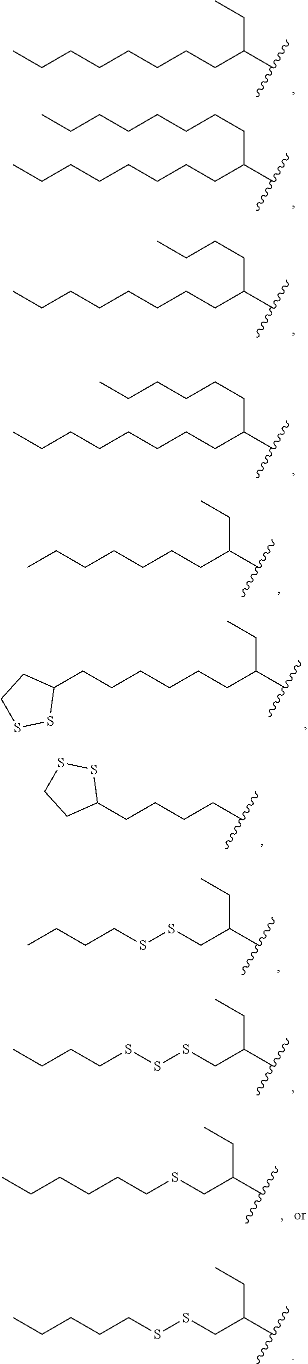

In some embodiments, R^{d3} and R^{e3} are each independently

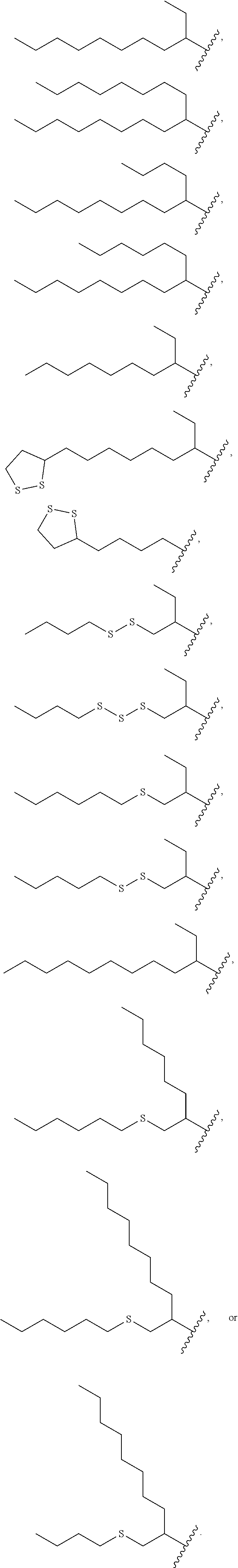

In some embodiments, R^{d3} and R^{e3} are the same.

In some embodiments, the compound or the salt thereof is selected from the group consisting of the compounds of Table 1 and salts thereof.

TABLE 1

| # | Lipid structure |
|---|---|
| 1 | 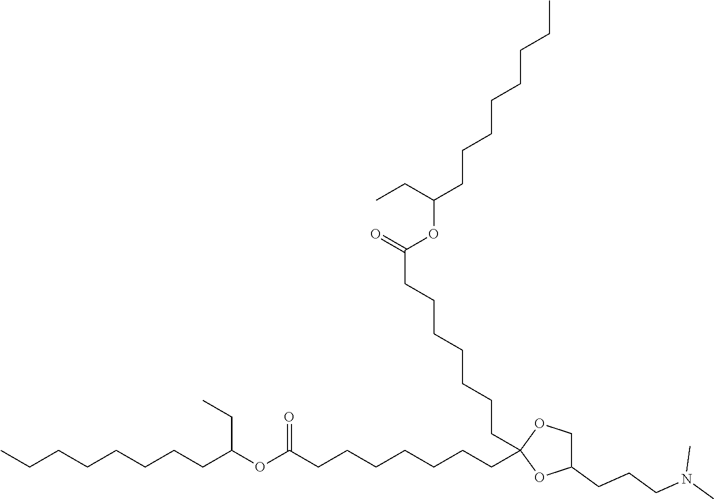 [SMILES: CCCCCCCCC(CC)OC(=O)CCCCCCCC1(CCCCCCCC(=O)OC(CC)CCCCCCCC)OCC(CCCN(C)C)O1] |
| 2 | 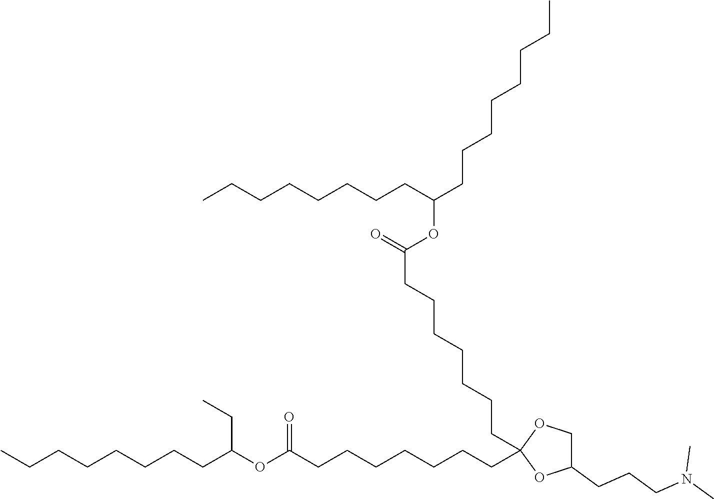 [SMILES: CCCCCCCCC(CC)OC(=O)CCCCCCCC1(CCCCCCCC(=O)OC(CCCCCCCC)CCCCCCCC)OCC(CCCN(C)C)O1] |
| 3 | 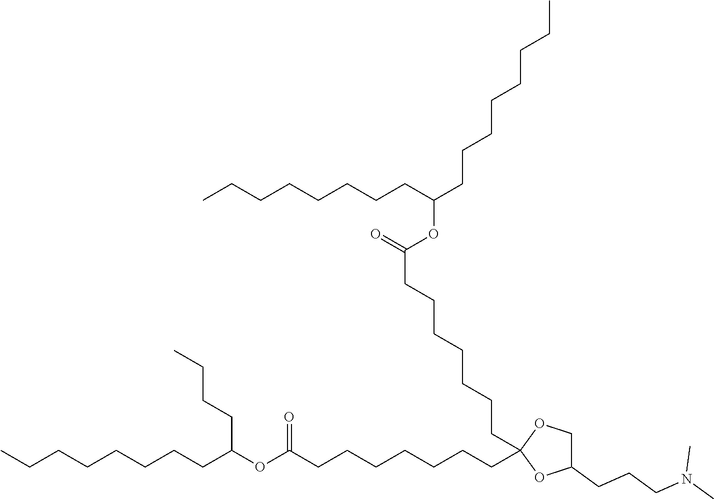 [SMILES: CCCCCCCCC(CCCC)OC(=O)CCCCCCCC1(CCCCCCCC(=O)OC(CCCCCCCC)CCCCCCCC)OCC(CCCN(C)C)O1] |
| 4 | 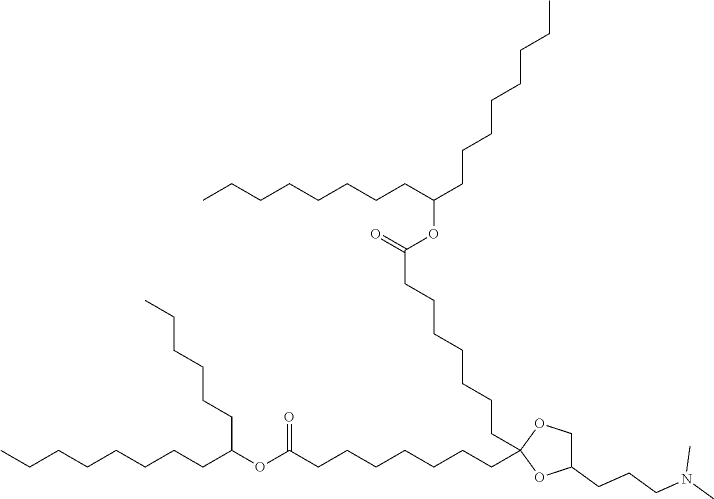 [SMILES: CCCCCCCCC(CCCCCC)OC(=O)CCCCCCCC1(CCCCCCCC(=O)OC(CCCCCCCC)CCCCCCCC)OCC(CCCN(C)C)O1] |
| 5 | 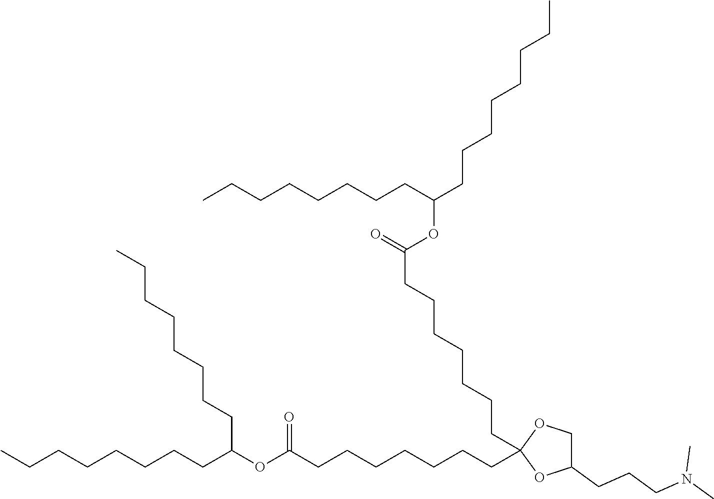 [SMILES: CCCCCCCCC(CCCCCCCC)OC(=O)CCCCCCCC1(CCCCCCCC(=O)OC(CCCCCCCC)CCCCCCCC)OCC(CCCN(C)C)O1] |
| 6 | 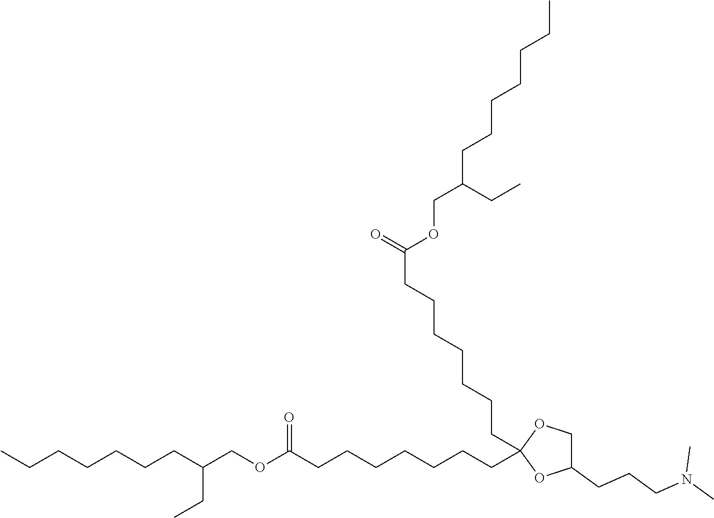 [SMILES: CCCCCCCC(CC)COC(=O)CCCCCCCC1(CCCCCCCC(=O)OCC(CC)CCCCCCC)OCC(CCCN(C)C)O1] |
| 7 | 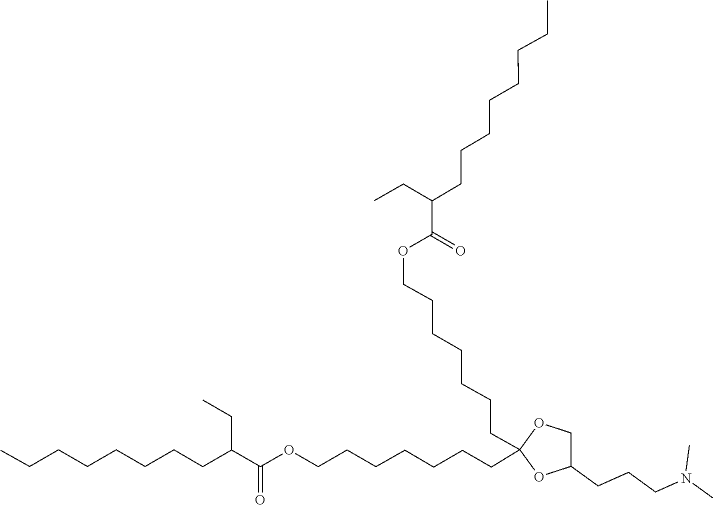 [SMILES: CCCCCCCCC(CC)C(=O)OCCCCCCCC1(CCCCCCCOC(=O)C(CC)CCCCCCCC)OCC(CCCN(C)C)O1] |
| 8 | 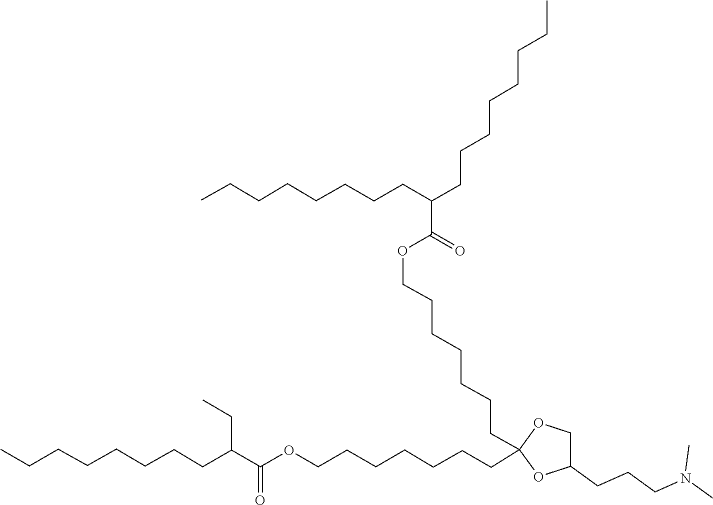 [SMILES: CCCCCCCCC(CC)C(=O)OCCCCCCCC1(CCCCCCCOC(=O)C(CCCCCCCC)CCCCCCCC)OCC(CCCN(C)C)O1] |
| 9 | 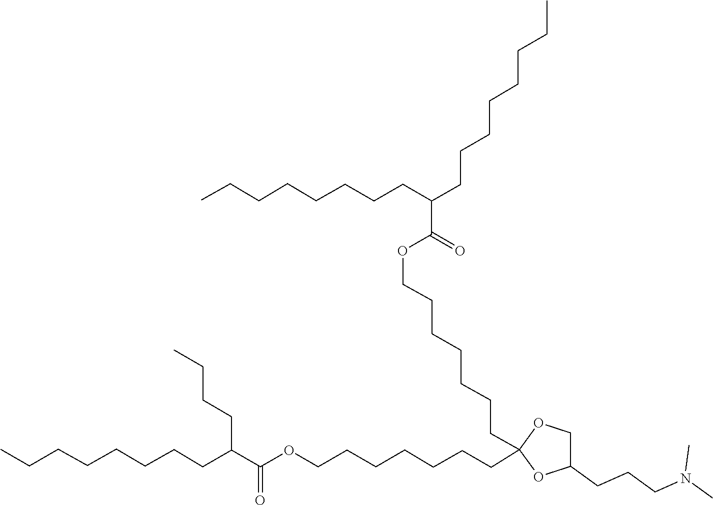 [SMILES: CCCCCCCCC(CCCC)C(=O)OCCCCCCCC1(CCCCCCCOC(=O)C(CCCCCCCC)CCCCCCCC)OCC(CCCN(C)C)O1] |
| 10 | 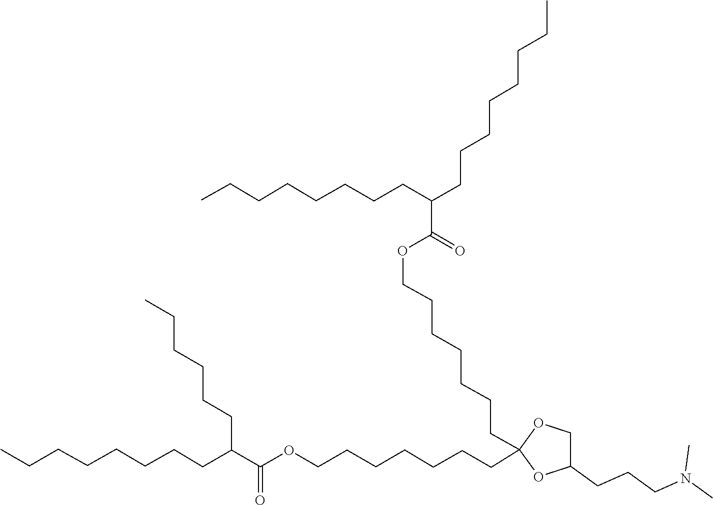 [SMILES: CCCCCCCCC(CCCCCC)C(=O)OCCCCCCCC1(CCCCCCCOC(=O)C(CCCCCCCC)CCCCCCCC)OCC(CCCN(C)C)O1] |
| 11 | 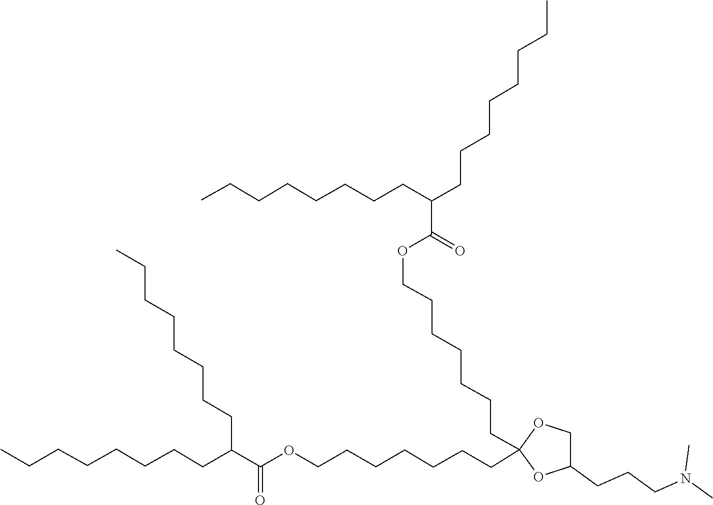 [SMILES: CCCCCCCCC(CCCCCCCC)C(=O)OCCCCCCCC1(CCCCCCCOC(=O)C(CCCCCCCC)CCCCCCCC)OCC(CCCN(C)C)O1] |
| 12 | 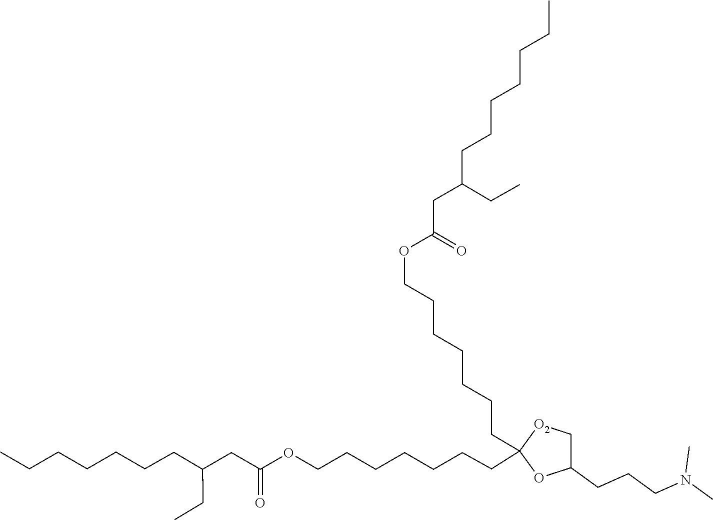 |
| 13 | 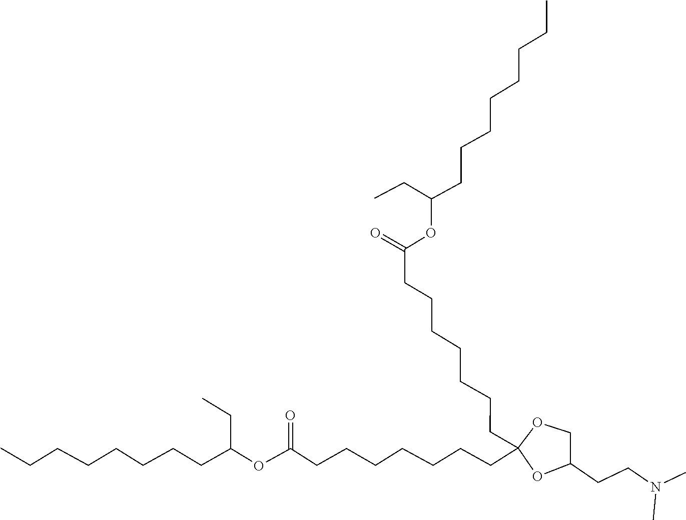 [SMILES: CCCCCCCCC(CC)OC(=O)CCCCCCCC1(CCCCCCCC(=O)OC(CC)CCCCCCCC)OCC(CCN(C)C)O1] |
| 14 | 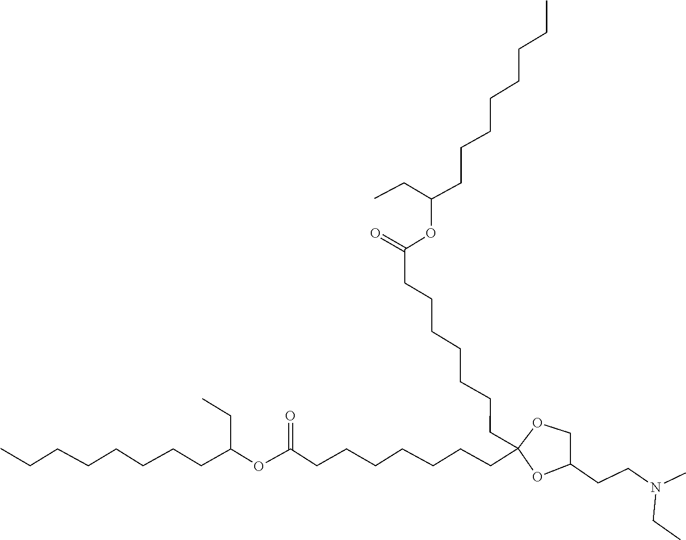 [SMILES: CCCCCCCCC(CC)OC(=O)CCCCCCCC1(CCCCCCCC(=O)OC(CC)CCCCCCCC)OCC(CCN(C)CC)O1] |
| 15 | 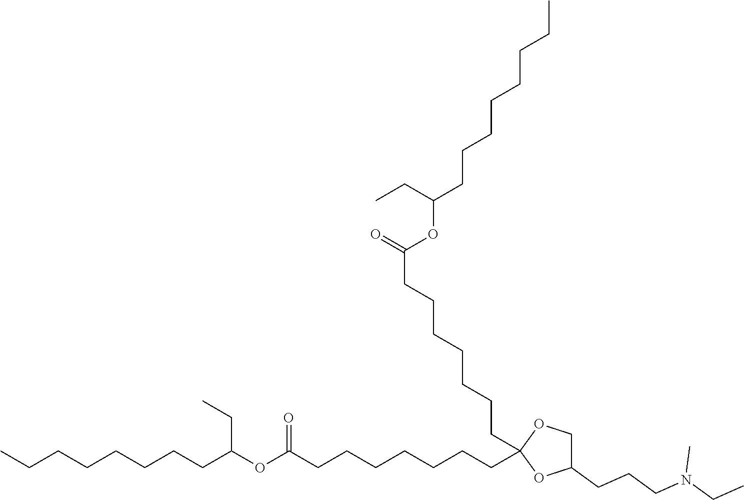 [SMILES: CCCCCCCCC(CC)OC(=O)CCCCCCCC1(CCCCCCCC(=O)OC(CC)CCCCCCCC)OCC(CCCN(C)CC)O1] |
| 16 | 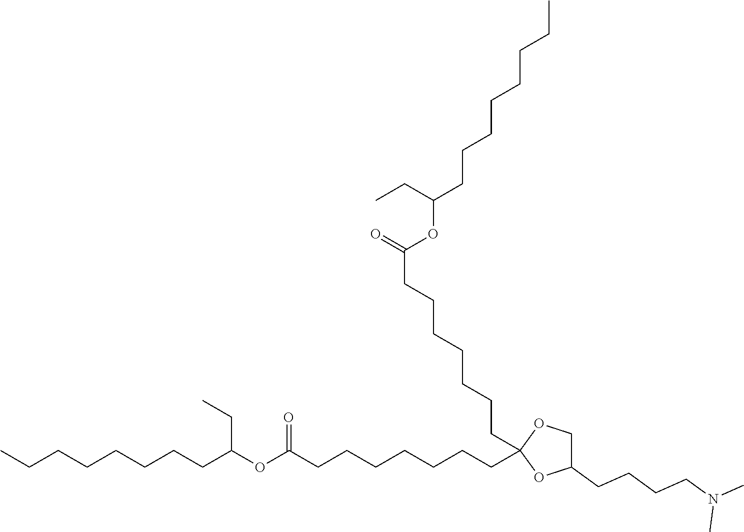 [SMILES: CCCCCCCCC(CC)OC(=O)CCCCCCCC1(CCCCCCCC(=O)OC(CC)CCCCCCCC)OCC(CCCCN(C)C)O1] |
| 17 | 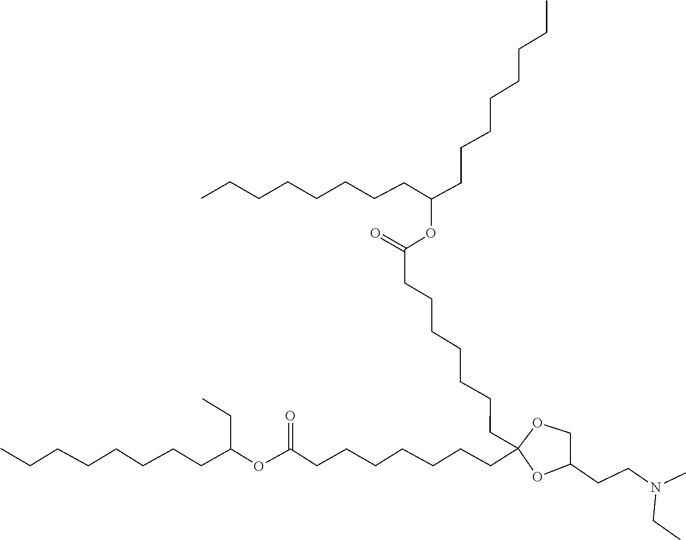 [SMILES: CCCCCCCCC(CC)OC(=O)CCCCCCCC1(CCCCCCCC(=O)OC(CCCCCCCC)CCCCCCCC)OCC(CCN(C)CC)O1] |
| 18 | 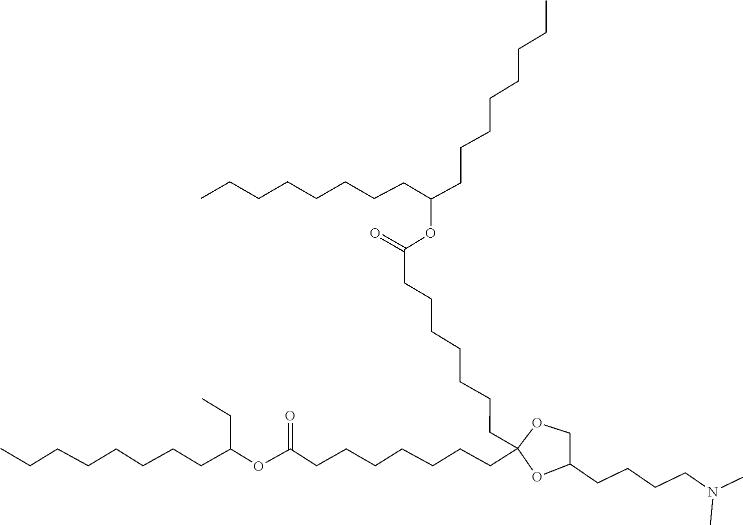 [SMILES: CCCCCCCCC(CC)OC(=O)CCCCCCCC1(CCCCCCCC(=O)OC(CCCCCCCC)CCCCCCCC)OCC(CCCCN(C)C)O1] |
| 19 | 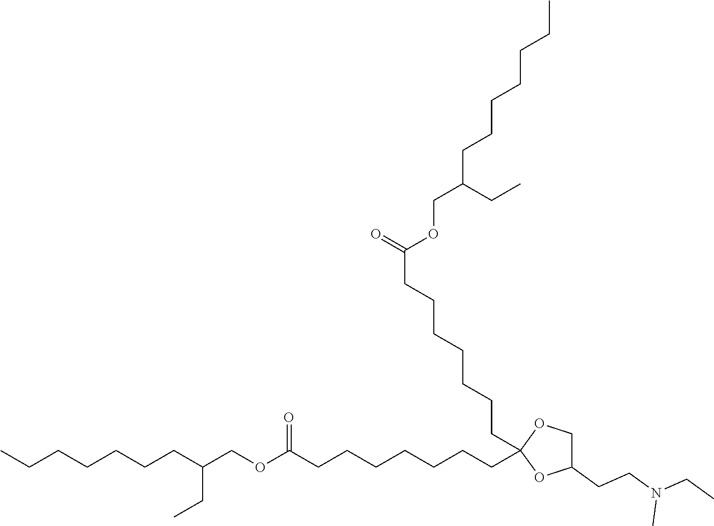 [SMILES: CCCCCCCC(CC)COC(=O)CCCCCCCC1(CCCCCCCC(=O)OCC(CC)CCCCCCC)OCC(CCN(C)CC)O1] |
| 20 | 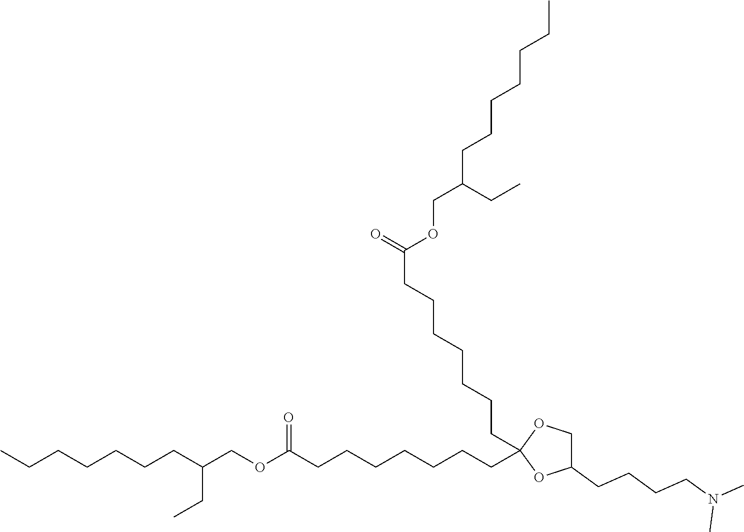 [SMILES: CCCCCCCC(CC)COC(=O)CCCCCCCC1(CCCCCCCC(=O)OCC(CC)CCCCCCC)OCC(CCCCN(C)C)O1] |
| 21 | 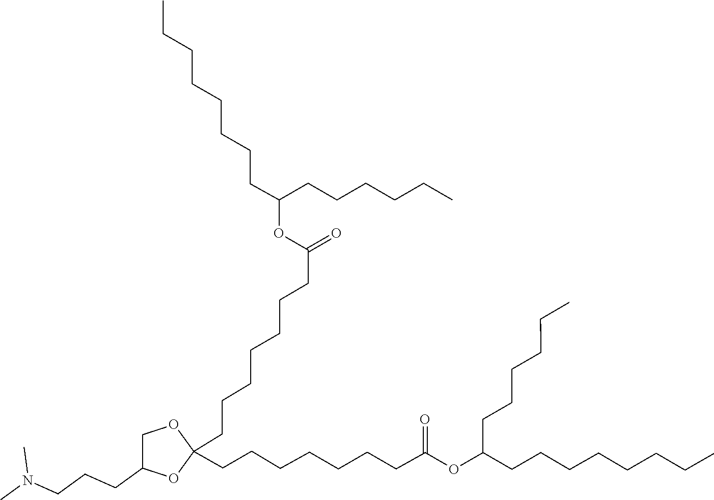 [SMILES: CCCCCCCCC(CCCCCC)OC(=O)CCCCCCCC1(CCCCCCCC(=O)OC(CCCCCC)CCCCCCCC)OCC(CCCN(C)C)O1] |
| 22 | 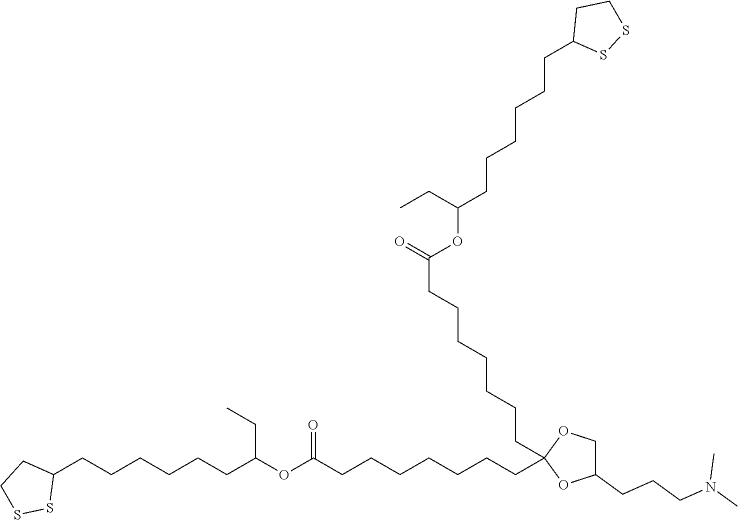 [SMILES: CCC(CCCCCCC1CCSS1)OC(=O)CCCCCCCC1(CCCCCCCC(=O)OC(CC)CCCCCCC2CCSS2)OCC(CCCN(C)C)O1] |
| 23 | 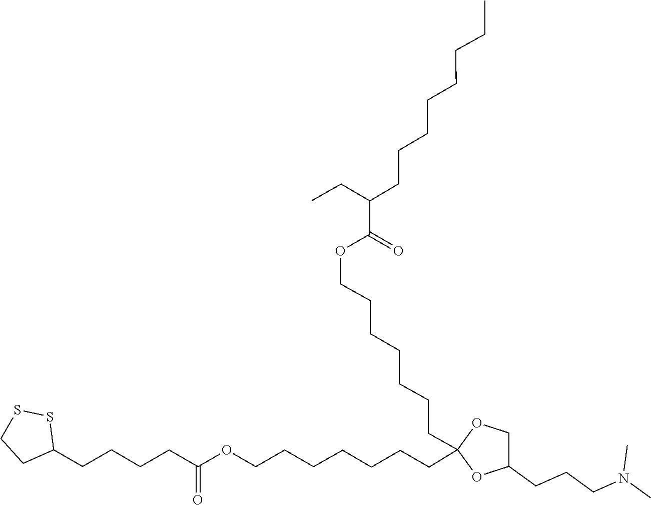 [SMILES: CCCCCCCCC(CC)C(=O)OCCCCCCCC1(CCCCCCCOC(=O)CCCCC2CCSS2)OCC(CCCN(C)C)O1] |
| 24 | 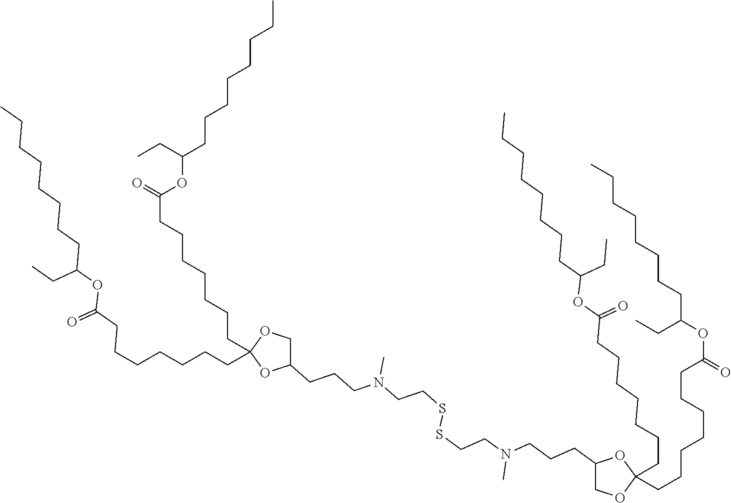 [SMILES: CCCCCCCCC(CC)OC(=O)CCCCCCCC1(CCCCCCCC(=O)OC(CC)CCCCCCCC)OCC(CCCN(C)CCSSCCN(C)CCCC2COC(CCCCCCCC(=O)OC(CC)CCCCCCCC)(CCCCCCCC(=O)OC(CC)CCCCCCCC)O2)O1] |
| 25 | 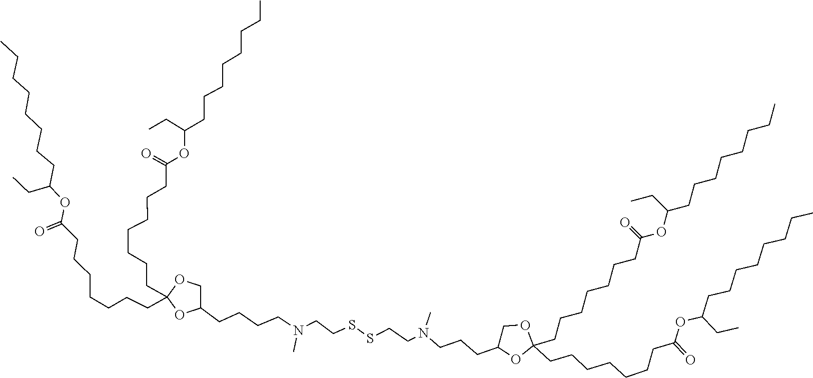 [SMILES: CCCCCCCCC(CC)OC(=O)CCCCCCCC1(CCCCCCCC(=O)OC(CC)CCCCCCCC)OCC(CCCCN(C)CCSSCCN(C)CCCC2COC(CCCCCCCC(=O)OC(CC)CCCCCCCC)(CCCCCCCC(=O)OC(CC)CCCCCCCC)O2)O1] |
| 26 | 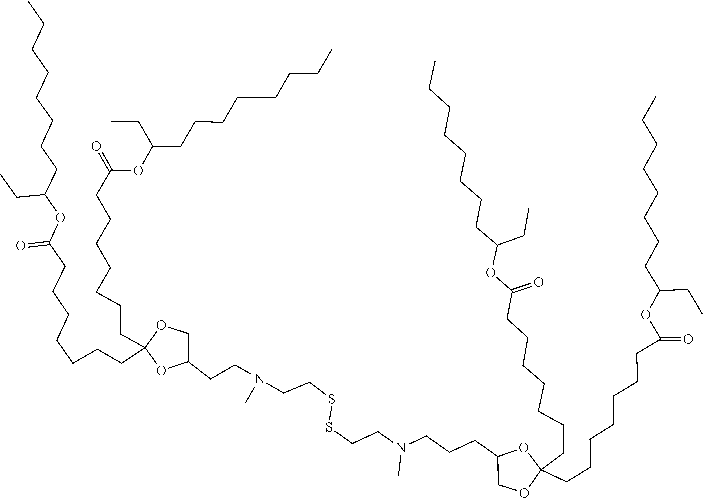 [SMILES: CCCCCCCCCC(CC)OC(=O)CCCCCCCC1(CCCCCCCC(=O)OC(CC)CCCCCCCC)OCC(CCN(C)CCSSCCN(C)CCCC2COC(CCCCCCCC(=O)OC(CC)CCCCCCCC)(CCCCCCCC(=O)OC(CC)CCCCCCCC)O2)O1] |
| 27 | 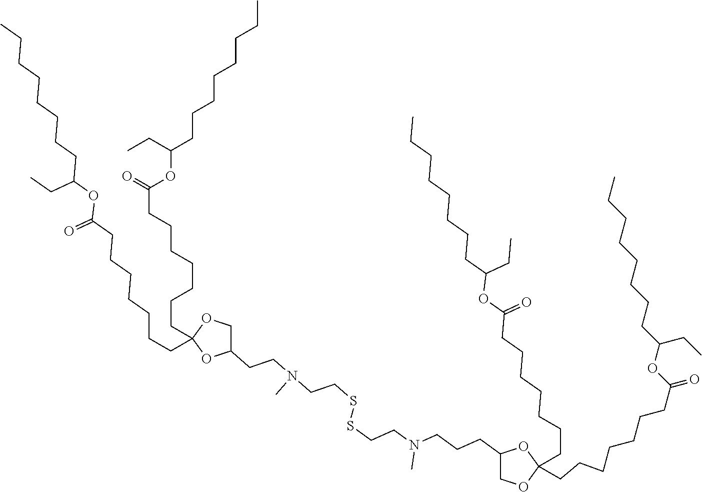 [SMILES: CCCCCCCCC(CC)OC(=O)CCCCCCCC1(CCCCCCCC(=O)OC(CC)CCCCCCCC)OCC(CCCN(C)CCSSCCN(C)CCC2COC(CCCCCCCC(=O)OC(CC)CCCCCCCC)(CCCCCCCC(=O)OC(CC)CCCCCCCC)O2)O1] |
| 28 | 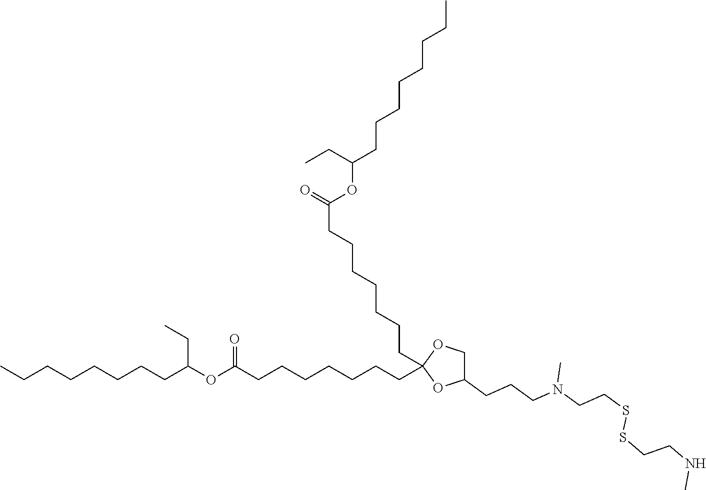 [SMILES: CCCCCCCCC(CC)OC(=O)CCCCCCCC1(CCCCCCCC(=O)OC(CC)CCCCCCCC)OCC(CCCN(C)CCSSCCNC)O1] |
| 29 | 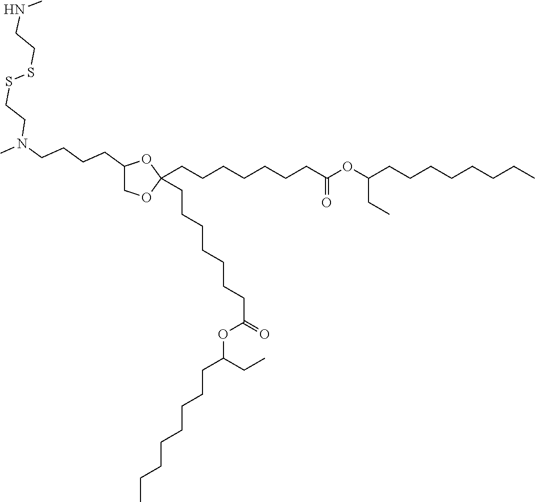 [SMILES: CCCCCCCCC(CC)OC(=O)CCCCCCCC1(CCCCCCCC(=O)OC(CC)CCCCCCCC)OCC(CCCCN(C)CCSSCCNC)O1] |
| 30 | 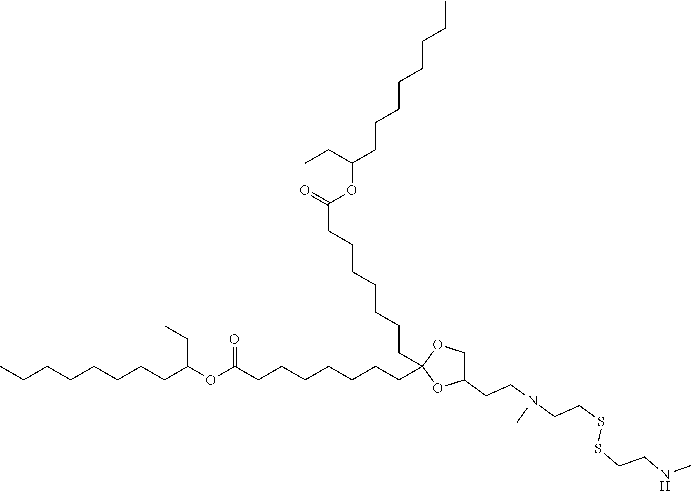 [SMILES: CCCCCCCCC(CC)OC(=O)CCCCCCCC1(CCCCCCCC(=O)OC(CC)CCCCCCCC)OCC(CCN(C)CCSSCCNC)O1] |
| 31 | 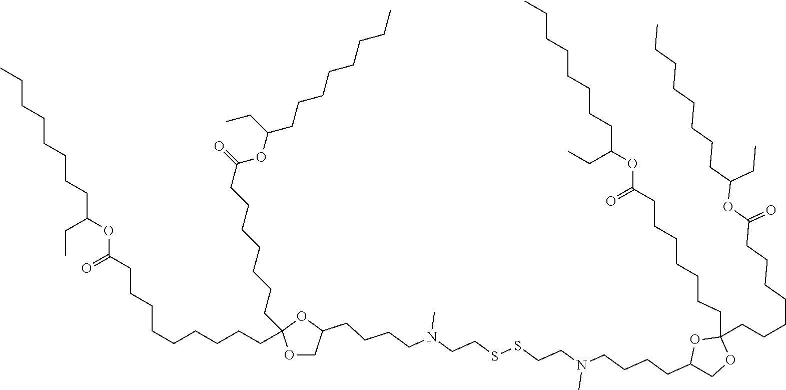 [SMILES: CCCCCCCCC(CC)OC(=O)CCCCCCCCCC1(CCCCCCCC(=O)OC(CC)CCCCCCCC)OCC(CCCCN(C)CCSSCCN(C)CCCCC2COC(CCCCCCCC(=O)OC(CC)CCCCCCCC)(CCCCCCCC(=O)OC(CC)CCCCCCCC)O2)O1] |
| 32 | 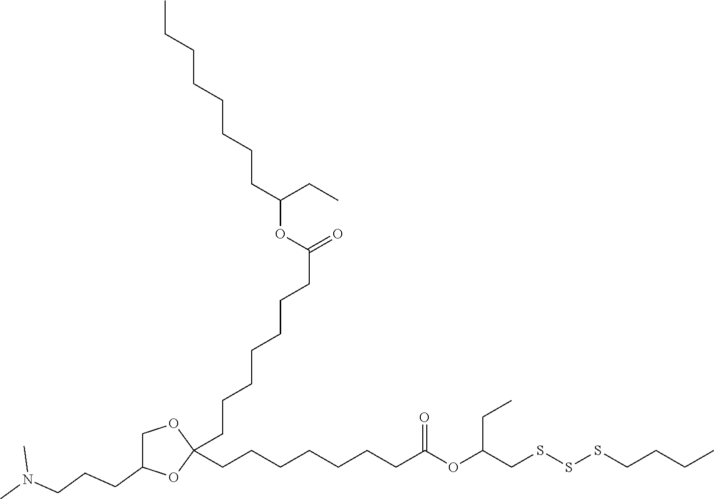 [SMILES: CCCCCCCCC(CC)OC(=O)CCCCCCCC1(CCCCCCCC(=O)OC(CC)CSSSCCCC)OCC(CCCN(C)C)O1] |
| 33 | 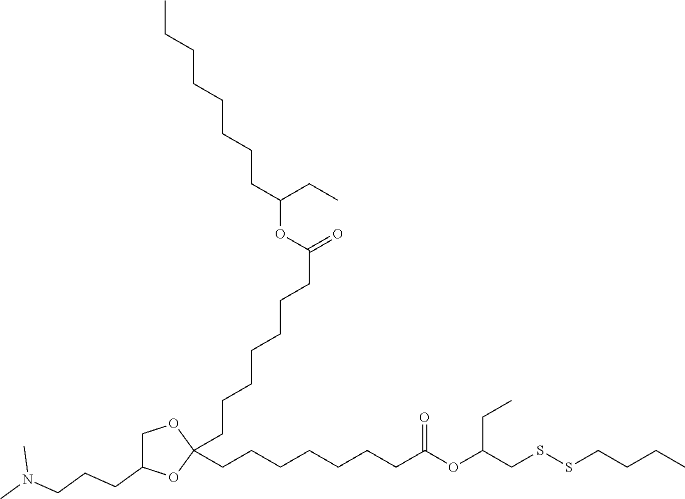 [SMILES: CCCCCCCCC(CC)OC(=O)CCCCCCCC1(CCCCCCCC(=O)OC(CC)CSSCCCC)OCC(CCCN(C)C)O1] |
| 34 | 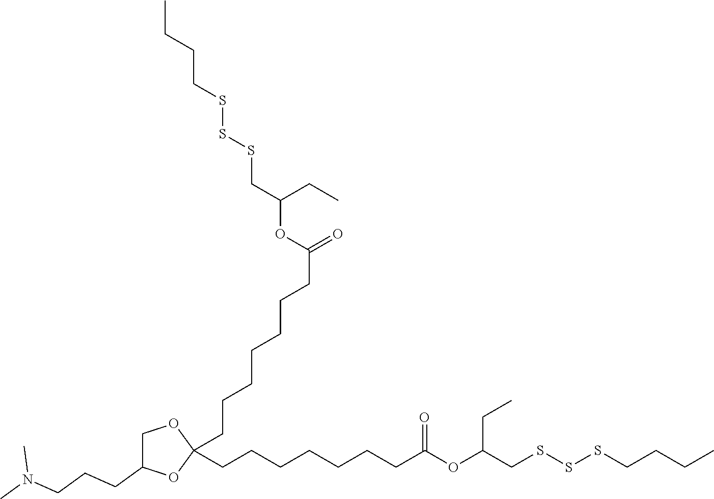 [SMILES: CCCCSSSCC(CC)OC(=O)CCCCCCCC1(CCCCCCCC(=O)OC(CC)CSSSCCCC)OCC(CCCN(C)C)O1] |
| 35 | 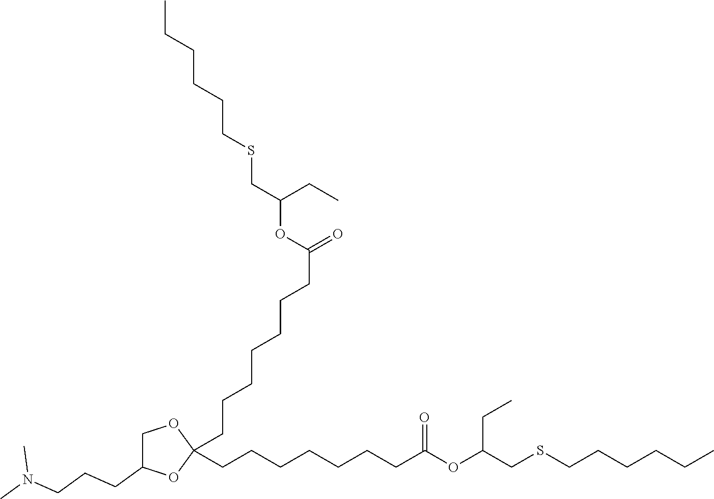 [SMILES: CCCCCCSCC(CC)OC(=O)CCCCCCCC1(CCCCCCCC(=O)OC(CC)CSCCCCCC)OCC(CCCN(C)C)O1] |
| 36 | 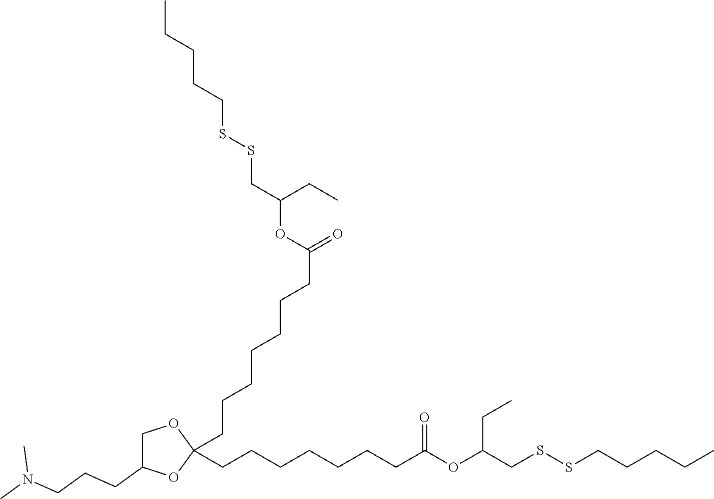 [SMILES: CCCCCSSCC(CC)OC(=O)CCCCCCCC1(CCCCCCCC(=O)OC(CC)CSSCCCCC)OCC(CCCN(C)C)O1] |
| 37 | 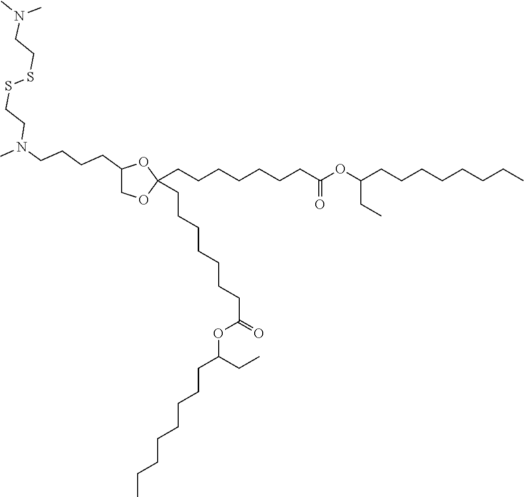 [SMILES: CCCCCCCCC(CC)OC(=O)CCCCCCCC1(CCCCCCCC(=O)OC(CC)CCCCCCCC)OCC(CCCCN(C)CCSSCCN(C)C)O1] |
| 38 | 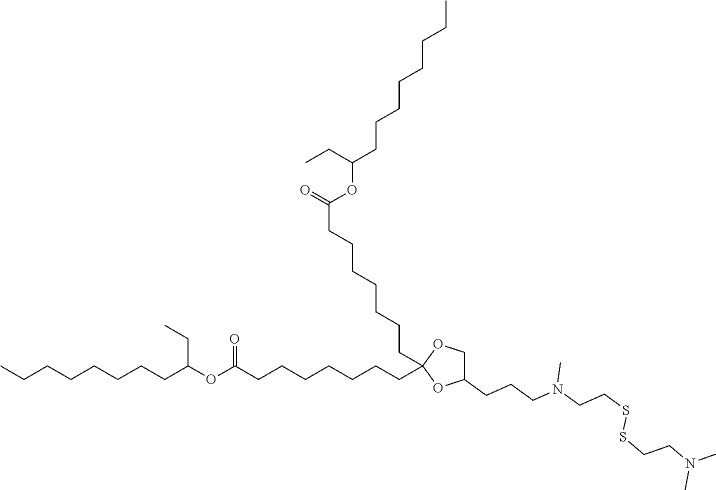 [SMILES: CCCCCCCCC(CC)OC(=O)CCCCCCCC1(CCCCCCCC(=O)OC(CC)CCCCCCCC)OCC(CCCN(C)CCSSCCN(C)C)O1] |
| 39 | 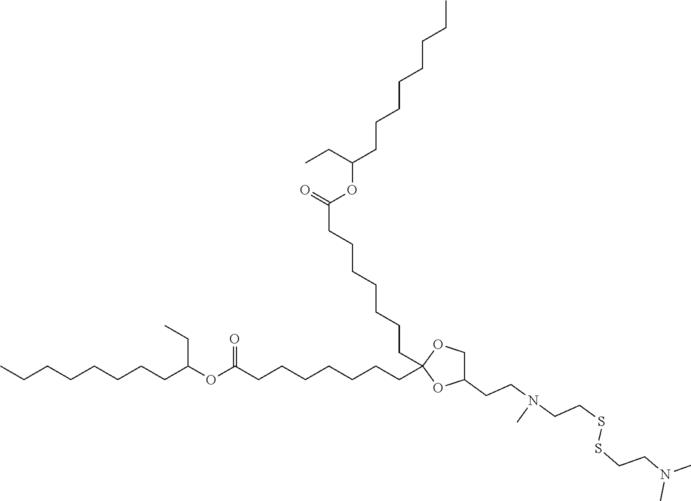 [SMILES: CCCCCCCCC(CC)OC(=O)CCCCCCCC1(CCCCCCCC(=O)OC(CC)CCCCCCCC)OCC(CCN(C)CCSSCCN(C)C)O1] |
| 40 | 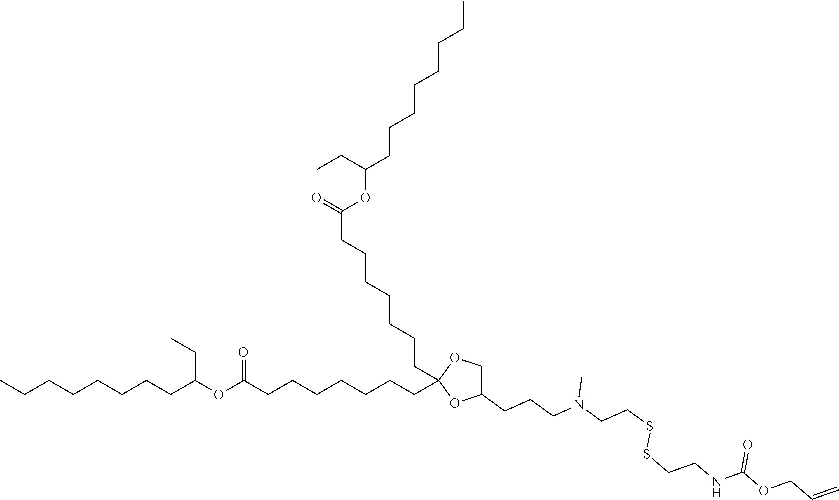 [SMILES: C=CCOC(=O)NCCSSCCN(C)CCCC1COC(CCCCCCCC(=O)OC(CC)CCCCCCCC)(CCCCCCCC(=O)OC(CC)CCCCCCCC)O1] |
| 41 | 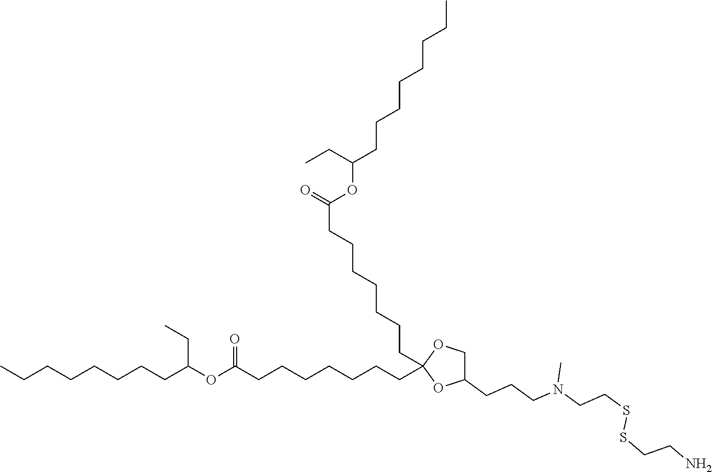 [SMILES: CCCCCCCCC(CC)OC(=O)CCCCCCCC1(CCCCCCCC(=O)OC(CC)CCCCCCCC)OCC(CCCN(C)CCSSCCN)O1] |
| 42 | 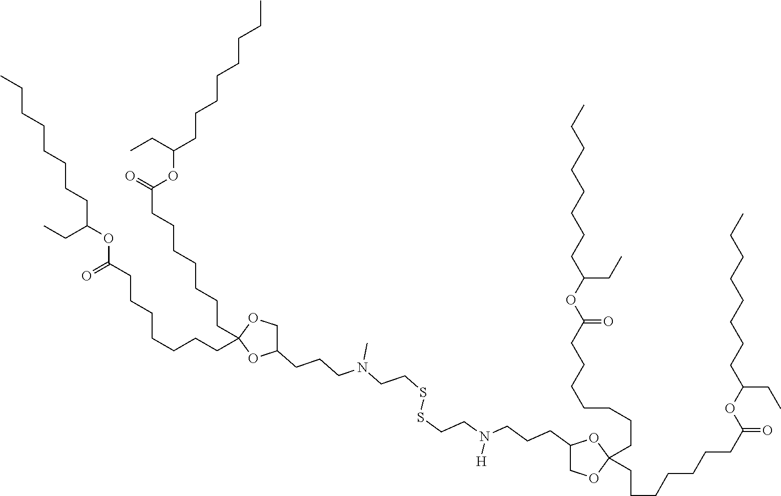 [SMILES: CCCCCCCCC(CC)OC(=O)CCCCCCCC1(CCCCCCCC(=O)OC(CC)CCCCCCCC)OCC(CCCNCCSSCCN(C)CCCC2COC(CCCCCCCC(=O)OC(CC)CCCCCCCC)(CCCCCCCC(=O)OC(CC)CCCCCCCC)O2)O1] |
| 43 | 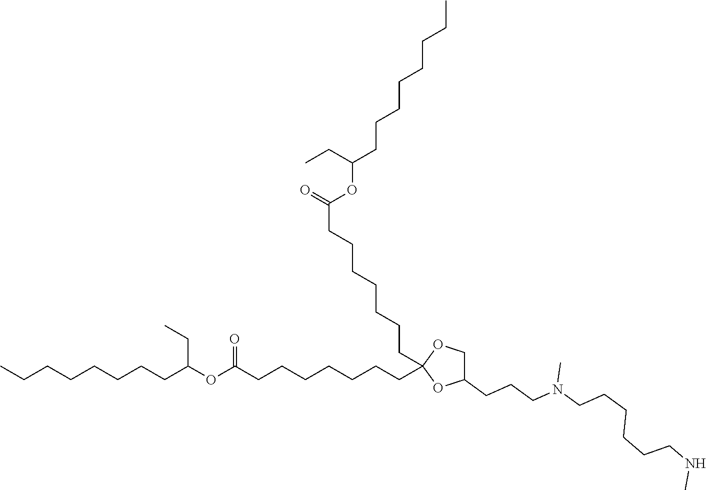 [SMILES: CCCCCCCCC(CC)OC(=O)CCCCCCCC1(CCCCCCCC(=O)OC(CC)CCCCCCCC)OCC(CCCN(C)CCCCCCNC)O1] |
| 44 | 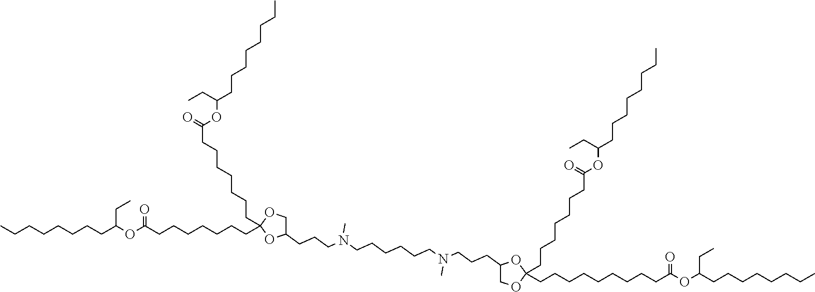 [SMILES: CCCCCCCCC(CC)OC(=O)CCCCCCCCCC1(CCCCCCCC(=O)OC(CC)CCCCCCCC)OCC(CCCN(C)CCCCCCN(C)CCCC2COC(CCCCCCCC(=O)OC(CC)CCCCCCCC)(CCCCCCCC(=O)OC(CC)CCCCCCCC)O2)O1] |
| 45 | 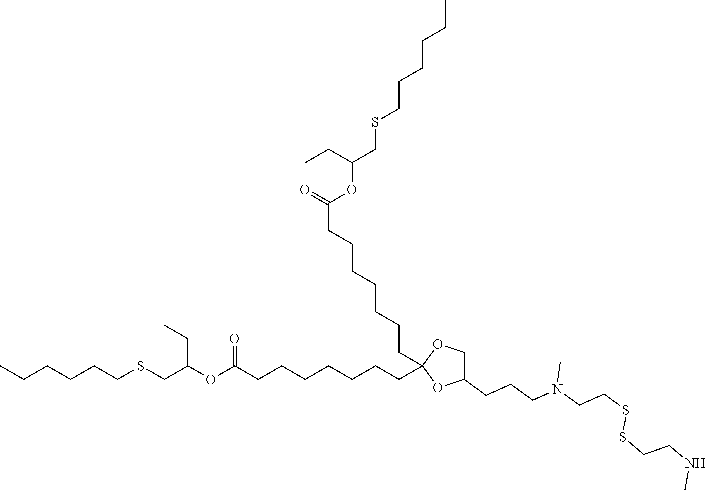 [SMILES: CCCCCCSCC(CC)OC(=O)CCCCCCCC1(CCCCCCCC(=O)OC(CC)CSCCCCCC)OCC(CCCN(C)CCSSCCNC)O1] |
| 46 | 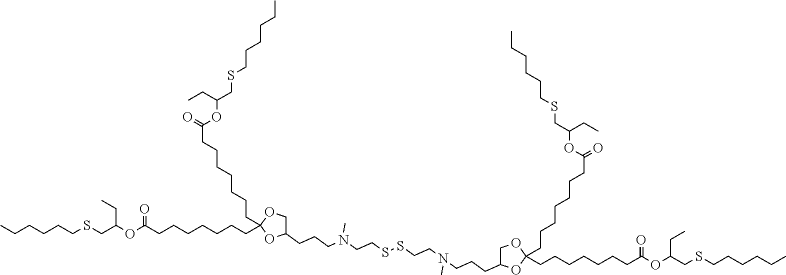 [SMILES: CCCCCCSCC(CC)OC(=O)CCCCCCCC1(CCCCCCCC(=O)OC(CC)CSCCCCCC)OCC(CCCN(C)CCSSCCN(C)CCCC2COC(CCCCCCCC(=O)OC(CC)CSCCCCCC)(CCCCCCCC(=O)OC(CC)CSCCCCCC)O2)O1] |
| 47 | 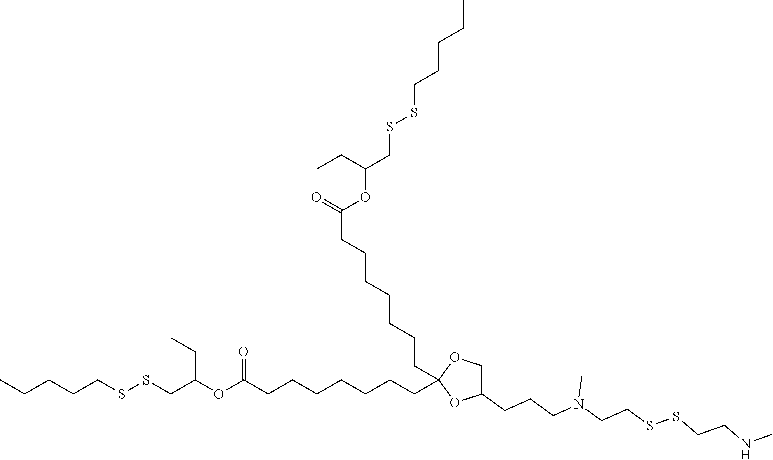 [SMILES: CCCCCSSCC(CC)OC(=O)CCCCCCCC1(CCCCCCCC(=O)OC(CC)CSSCCCCC)OCC(CCCN(C)CCSSCCNC)O1] |
| 48 | 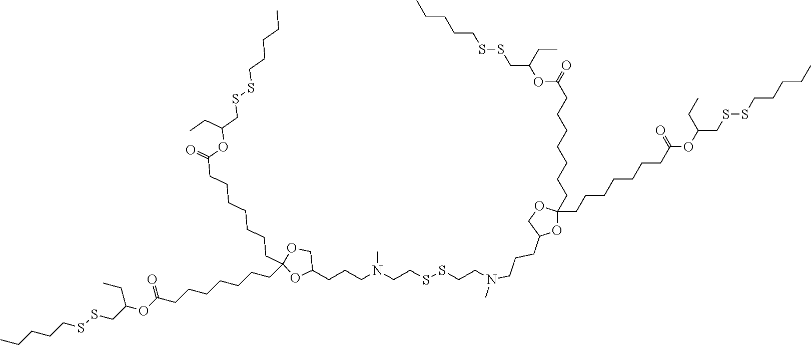 [SMILES: CCCCCSSCC(CC)OC(=O)CCCCCCCC1(CCCCCCCC(=O)OC(CC)CSSCCCCC)OCC(CCCN(C)CCSSCCN(C)CCCC2COC(CCCCCCCC(=O)OC(CC)CSSCCCCC)(CCCCCCCC(=O)OC(CC)CSSCCCCC)O2)O1] |
| 49 | 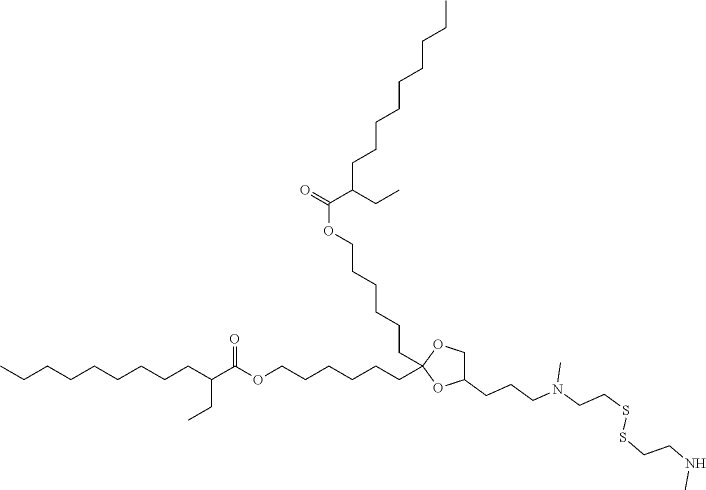 [SMILES: CCCCCCCCCC(CC)C(=O)OCCCCCCC1(CCCCCCOC(=O)C(CC)CCCCCCCCC)OCC(CCCN(C)CCSSCCNC)O1] |
| 50 | 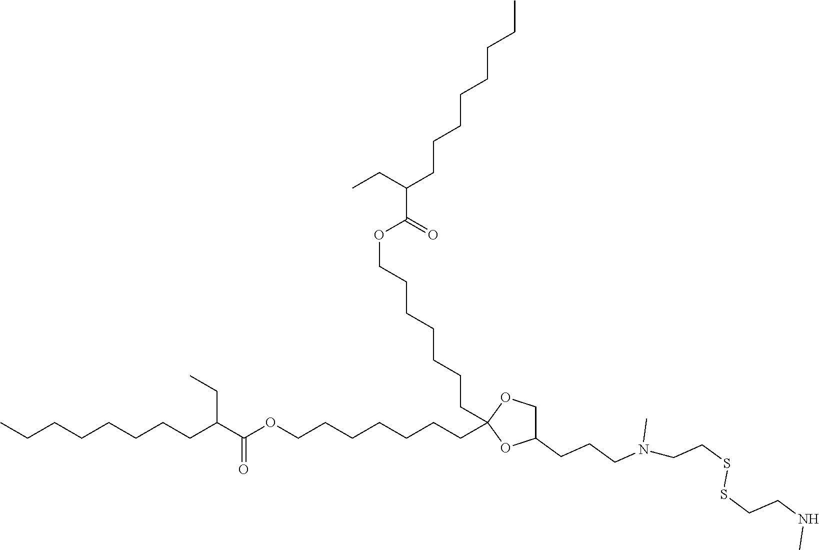 [SMILES: CCCCCCCCC(CC)C(=O)OCCCCCCCC1(CCCCCCCOC(=O)C(CC)CCCCCCCC)OCC(CCCN(C)CCSSCCNC)O1] |
| 51 | 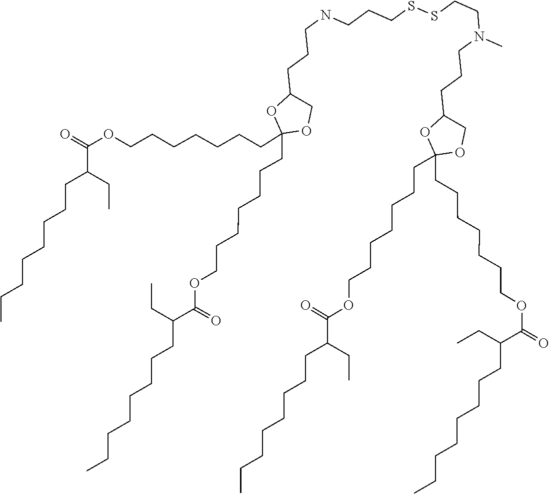 [SMILES: CCCCCCCCC(CC)C(=O)OCCCCCCCC1(CCCCCCCOC(=O)C(CC)CCCCCCCC)OCC(CCCNCCCSSCCN(C)CCCC2COC(CCCCCCCOC(=O)C(CC)CCCCCCCC)(CCCCCCCOC(=O)C(CC)CCCCCCCC)O2)O1] |
| 52 | 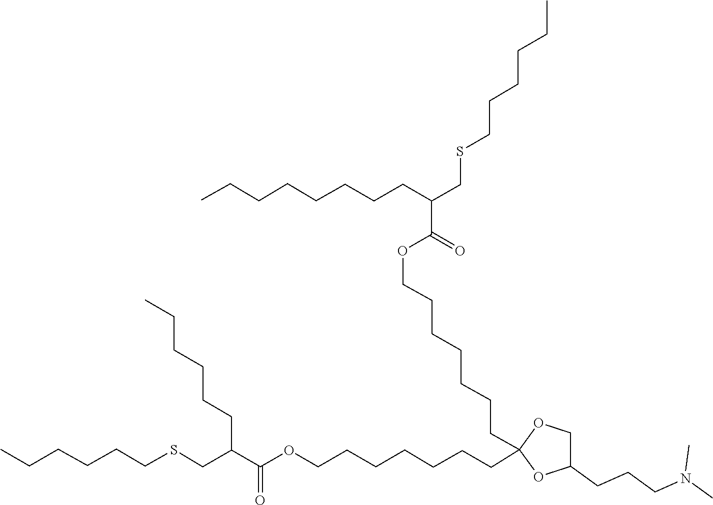 [SMILES: CCCCCCCCC(CSCCCCCC)C(=O)OCCCCCCCC1(CCCCCCCOC(=O)C(CCCCCC)CSCCCCCC)OCC(CCCN(C)C)O1] |
| 53 | 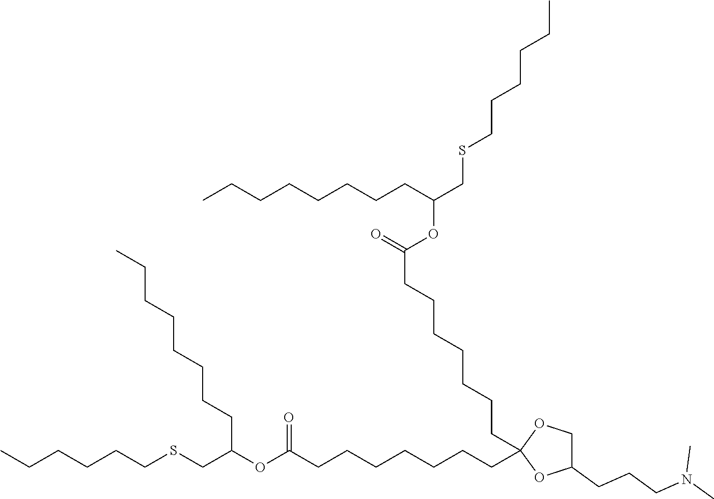 [SMILES: CCCCCCCCC(CSCCCCCC)OC(=O)CCCCCCCC1(CCCCCCCC(=O)OC(CCCCCCCC)CSCCCCCC)OCC(CCCN(C)C)O1] |
| 54 | 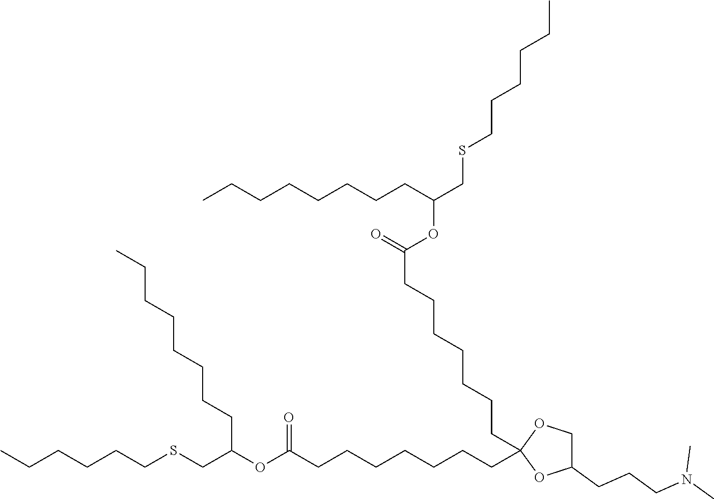 [SMILES: CCCCCCCCC(CSCCCCCC)OC(=O)CCCCCCCC1(CCCCCCCC(=O)OC(CCCCCCCC)CSCCCCCC)OCC(CCCN(C)C)O1] |
| 55 | 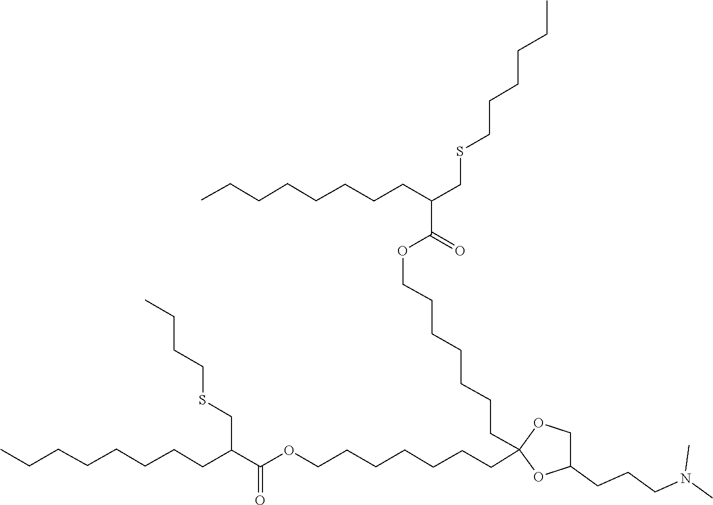 [SMILES: CCCCCCCCC(CSCCCC)C(=O)OCCCCCCCC1(CCCCCCCOC(=O)C(CCCCCCCC)CSCCCCCC)OCC(CCCN(C)C)O1] |

Any variation or embodiment of R^{a1}, R^{b1}, X^{a}, X^{b}, R^{a2}, R^{b2}, R^{a3}, R^{b3}, R^{a3a}, R^{a3b}, R^{b3a}, R^{b3b}, R^{c1}, R^{c2}, R^{c3}, R^{d1}, R^{e1}, X^{d}, X^{e}, R^{d2}, R^{e2}, R^{d3}, R^{e3}, R^{d3a}, R^{d3b}, R^{e3a}, R^{e3b}, R^{f1}, R^{f2}, R^{f3}, R^{f4}, or R^{f5} provided herein can be combined with every other variation or embodiment of R^{a1}, R^{b1}, X^{a}, X^{b}, R^{a2}, R^{b2}, R^{a3}, R^{b3}, R^{a3a}, R^{a3b}, R^{b3a}, R^{b3b}, R^{c1}, R^{c2}, R^{c3}, R^{d1}, R^{e1}, X^{d}, X^{e}, R^{d2}, R^{e2}, R^{d3}, R^{e3}, R^{d3a}, R^{d3b}, R^{c3a}, R^{c3b}, R^{f1}, R^{f2}, R^{f3}, R^{f4}, or R^{f5} as if each combination bad been individually and specifically described.

### III. Lipid-Cell Targeting Group Conjugates

As discussed herein, the LNPs may be targeted to a particular cell type, e.g., an immune cell, e.g., a T cell, B cell, natural killer (NK) cell, macrophages, monocytes, or dendritic cells, or a hematopoietic stem cell. This can be accomplished by using one or more of the lipids described herein. Furthermore, targeting can be enhanced by including a targeting group at a solvent accessible surface of an LNP particle. For example, targeting groups may include a member of a specific binding pair, e.g., an antibody-antigen pair, a ligand-receptor pair, etc. In certain embodiments, the targeting group is an antibody. Targeting can be implemented, for example, by using lipid-cell targeting group conjugates described herein.

Optionally, the targeting moiety is an antibody fragment without an Fc component. Previous attempts to target circulating immune cells with LNPs have employed full antibodies (WO 2016/189532 A1). Liposomes or lipid based particles with conjugated full antibodies clear more quickly from the circulation due to engagement of the Fc, reducing their potential for reaching the target cell of interest (Harding et al. (1997) Biochim Biophys. Acta 1327, 181-192; Sapra et al. (2004) Clin Cancer Res 10, 1100-1111; Aragnol et al., (1986) Proc Natl Acad Sci USA 83, 2699-2703). Liposomes targeted with antibody fragments retain their long circulating properties, like those targeted to EGFR (Mamot et al., (2005) Cancer Res 65, 11631-11638), ErbB2 (Park et al. (2002) Clin Cancer Res 8, 1172-1181), or EphA2 (Kamoun et al., 2019 Nat. Biomed. Eng 3, 264-280). In addition, lipid based carriers can be prepared using a micellar insertion process that allows for the nearly quantitative incorporation of the antibody conjugation following its separate manufacturing (Nellis et al. (2005) Biotechnol Prog 21, 221-232), compared to a highly inefficient insertion when conjugating full IgGs (Ishida et al. (1999) FEBS Lett. 460, 129-133) or the need to complete conjugation directly on an intact LNP (WO 2016/189532 A1). scFv, Fab, or V_{HH} fragments can also be directly conjugated to activated PEG-lipids to make insertable conjugates.

In some embodiments, PEG-(lipid) is equivalent to (lipid)-PEG.

In certain embodiments, a targeting group may be a surface-bound antibody or surface bound antigen binding fragment thereof, which can permit tuning of cell targeting specificity. This is especially useful since highly specific antibodies can be raised against an epitope of interest for the desired targeting site. In one embodiment, multiple different antibodies can be incorporated into, and presented at the surface of an LNP, where each antibody binds to different epitopes on the same antigen or different epitopes on different antigens. Such approaches can increase the avidity and specificity of targeting interactions to a particular target cell.

A targeting group or combination of targeting groups can be selected based on the desired localization, function, or structural features of a given target cell. For example, in order to target a T-cell, T-cell population or T-cell subpopulation, one or more antibodies or antigen binding fragments or antigen binding derivatives thereof may be selected that target a T-cell, such as via a T-cell surface antigen. Exemplary T-cell surface antigens include, but are not limited to, for example, CD2, CD3, CD4, CD5, CD7, CD8, CD28, CD39, CD69, CD103, CD137, CD45, T-cell receptor (TCR) β, TCR-a, TCR-a/b,TCR-g/d, PD1, CTLA4, TIM3, LAG3, CD18, IL-2 receptor, CD11a, GL7, TLR2, TLR4, TLR5 and IL-15 receptor. In order to target an NK cell, or NK cell population, one or more antibodies, antigen binding fragments or antigen binding derivatives thereof may be selected that target an NK cell such as via a NK cell surface antigen. Exemplary NK cell surface antigens include, but are not limited to, CD48, CD56, CD85a, CD85c, CD85d, CD85e, CD85f, CD85i, CD85j, CD158b2, CD161, CD244, CD16a, CD16b, IL-2 receptor, CD27, CD28, CD48, CD69, CD70, CD86, CD112, CD122, CD155, CD161, CD244, CD266, CD314/NKG2D, CD336/NKP44, CD337/NKP30. In order to target a B cell or B cell population, one or more antibodies, antigen binding fragments or antigen binding derivatives thereof may be selected that target a B cell such as via a B cell antigen. Exemplary B cell antigens include, but are not limited to, CD19 for all B cells except plasma cells, CD19, CD25, and CD30 for activated B cells, CD27, CD38, CD78, CD138, and CD319 for plasma cells, CD20, CD27, CD40, CD80 and PDL-2 for memory cells, Notch2, CD1, CD21, and CD27 for marginal zone B cells, CD21, CD22, and CD23 for follicular B cells, and CD1, CD5, CD21, CD24, and TLR4 for regulatory B cells.

In order to target a macrophage, macrophage polulation or macrophage subpopulation, one or more antibodies or antigen binding fragments or antigen binding derivatives thereof may be selected that target a macrophage, such as via a macrophage surface antigen. In some embodiments, the antigen is a M1 macrophage specific antigen. In some embodiments, the antigen is a M2 macrophage specific antigen. Exemplary macrophage surface antigens include, but are not limited to, for example, CDIIB, CD80, CD86, HLA, CD68, CD163, CD206. In some embodiments, tumor macrophages are targeted, and the antigen is CD206.

In order to target a monocyte, monocyte population or monocyte subpopulation, one or more antibodies or antigen binding fragments or antigen binding derivatives thereof may be selected that target a monocyte, such as via a monocyte surface antigen. Exemplary monocyte surface antigens include, but are not limited to, for example, CD14, CCR2, CCR5, CD62L, HLA, CD68, CXCR1, CXCR3, and CD11c.

In order to target a dendritic cell, dendritic cell population or dendritic subpopulation, one or more antibodies or antigen binding fragments or antigen binding derivatives thereof may be selected that target a dendritic cell, such as via a dendritic surface antigen. Exemplary dendritic surface antigens include, but are not limited to, for example, DEC205 (see Katakowski, 2016:24(1):146-155, Molecular Therapy).

In order to target a hematopoietic stem cell, one or more antibodies or antigen binding fragments or antigen binding derivatives thereof may be selected that target a hematopoietic stem cell, such as via a hematopoietic stem cell surface antigen. Exemplary hematopoietic stem cell surface antigens include, but are not limited to, CD34, CD105 (also known as endoglin), or CD117 (also known as c-kit, tyrosine-protein kinase KIT, or mast/stem cell growth factor receptor (SCFR)).

In certain embodiments, targeting can be implemented, for example, by using lipid-immune cell targeting group conjugates described herein. Exemplary lipid-immune cell targeting group conjugates can include compounds of Formula (II),

$$[ Lipid ] - [ optional linker ] - [ immune cell targeting group , e . g . , T - cell or macrophage targeting molecule , e . g . , anti - CD 2 antibody , anti - CD 3 antibody , anti - CD 7 antibody , anti - CD 8 antibody , anti - CD IIB antibody , anti - CD 80 antibody , anti - CD 86 antibody , anti - CD 68 antibody , anti - CD 163 antibody , and / or anti - CD 206 antibody ] . \qquad ( Formula II )$$

In some embodiments, the immune cell targeting group is a polypeptide, and the lipid is conjugated to the N-terminus, C-terminus, or anywhere in the middle part of the polypeptide.

In certain embodiments, the targeting group or targeting molecule is a T-cell targeting agent, for example, an antibody, that binds to a T-cell antigen selected from the group consisting of CD2, CD3, CD4, CD5, CD7, CD8, CD28, CD137, CD45, T-cell receptor (TCR)β, TCR-a, TCR-a/b, TCR-g/d, PD1, CTLA4, TIM3, LAG3, CD18, IL-2 receptor, CD11a, TLR2, TLR4, TLR5, IL-7 receptor, or IL-15 receptor. In certain embodiments, the T cell antigen may be CD2, and the targeting group can be, for example, an anti-CD2 antibody. In certain embodiments, the T cell antigen may be CD3, and the targeting group can be, for example, an anti-CD3 antibody. In certain embodiments, the T cell antigen may be CD4, and the targeting group can be, for example, an anti-CD4 antibody. In certain embodiments, the T cell antigen may be CD5, and the targeting group can be, for example, an anti-CDS antibody. In certain embodiments, the T cell antigen may be CD7, and the targeting group can be, for example, an anti-CD7 antibody. In certain embodiments, the T cell antigen may be CD8, and the targeting group can be, for example, an anti-CD8 antibody. In certain embodiments, the T cell antigen may be TCR β, and the targeting group can be, for example, an anti-TCR. B antibody. In some embodiments, the antibody is a human or humanized antibody. In some embodiments, the antibody is an antibody fragments, such as a Fab, a VHH, or an scFv.

In certain embodiments, targeting can be implemented, for example, by using lipid-cell targeting group conjugates described herein. Exemplary lipid-cell targeting group conjugates can include compounds of Formula (V),

[Lipid]-[optional linker]-[cell targeting group], wherein the cell targeting group, binds to a molecule on a hematopoietic stem cell. In some embodiments, the cell targeting group is a polypeptide, and the lipid is conjugated to the N-terminus, C-terminus, or anywhere in the middle part of the polypeptide. In certain embodiments, the hematopoietic cell targeting group is an antibody that binds to a hematopoietic stem cell antigen selected from the group consisting of CD34, CD105, or CD117. In certain embodiments, the hematopoietic stem cell antigen may be CD34, and the targeting group can be, for example, an anti-CD34 antibody. In certain embodiments, the hematopoietic stem cell antigen may be CD105, and the targeting group can be, for example, an anti-CD105 antibody. In certain embodiments, the hematopoietic stem cell antigen may be CD117, and the targeting group can be, for example, an anti-CD117 antibody. In some embodiments, the antibody is a human or humanized antibody. In some embodiments, the antibody is an antibody fragments, such as a Fab, a VHH, or an scFv.

An exemplary CD2 binding agent can be an antibody selected from the group consisting of 9.6 (https://academic.oup.com/intimm/article/10/12/1863/744536; Connelly et al., International Immunology, Volume 10, Issue 12, December 1998, pages 1863-1872), 9-1 (https://academic.oup.com/intimm/article/10/12/1863/744536; Connelly et al., International Immunology, Volume 10, Issue 12, December 1998, pages 1863-1872), TS2/18.1.1 (ATCC HB-195), Lo-CD2b (ATCC PTA-802), Lo-CD2a/BTI-322 (U.S. Pat. No. 6,849,258B1), Sipilzumab/MEDI-507 (U.S. Pat. No. 6,849,258B1/en), 35.1 (ATCC HB-222), OKT11 (ATCC CRL-8027), RPA-2.1 (PCT Publication WO2020023559A1), AF1856 (R&D Systems), MAB18562 (R&D Systems), MAB18561 (R&D Systems), MAB1856 (R&D Systems), PAB30359 (Abnova Corporation), 10299-1 (Abnova Corporation), and antigen binding fragments thereof. In certain embodiments, the binding agent comprises a heavy chain variable domain (V_{H}) and a light chain variable domain (V_{L}) of an antibody selected from the group consisting of AF1856 (R&D Systems), MAB18562 (R&D Systems), MAB18561 (R&D Systems), MAB1856 (R&D Systems), PAB30359 (Abnova Corporation), and 10299-1 (Abnova Corporation). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL. BIOL. 262:732-745), or any other CDR determination method known in the art, of the V_{H} and V_{L} sequences of an antibody selected from the group consisting of AF1856 (R&D Systems), MAB18562 (R&D Systems), MAB18561 (R&D Systems), MAB1856 (R&D Systems), PAB30359 (Abnova Corporation), and 10299-1 (Abnova Corporation).

An exemplary CD2 binding agent can also be selected from antibodies or antibody fragments employing CDRs of clones 9.6, 9-1, TS2/18.1.1, Lo-CD2b, Lo-CD2a, BTI-322, sipilzumab, 35.1, OKT11, RPA-2.1, SQB-3.21, LT2, TS1/8, UT329, 4F22, OX-34, UQ2/42, MU3, U7.4, NFN-76, or MOM-181-4-F (E).

An exemplary CD3 binding agent (CD3γ/δ/ε, CD3γ, CD3δ, CD3γ/ε, CD3δ/ε, or CD3ε) can be an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3δ, Santa Cruz Biotechnology, Inc.), NBP1-31250 (CD3γ, Novus Biologicals), 16669-1-AP (CD3δ, Invitrogen) and antigen binding fragments thereof. In certain embodiments, the binding agent comprises a V_{H} domain and a V_{L} domain of an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3δ, Invitrogen). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL, BIOL. 262:732-745), or any other CDR determination method known in the art, of the V_{H} and V_{L} sequences of an antibody selected from the group consisting of MEM-57 (CD3γ/δ/ε, EnzoLife Sciences), MAB100 (CD3ε, R&D Systems), CD3-H5 (CD3ε, Abnova Corporation), CD3-12 (CD3ε, Cell Signaling Technology), LE-CD3 (CD3ε, Santa Cruz Biotechnology, Inc.), NBP1-31250 (CD3γ, Novus Biologicals), and 16669-1-AP (CD3δ, Invitrogen).

An exemplary CD3 binding agent can also be selected from antibodies or antibody fragments employing CDRs of clones hsp34, OKT-3, UCHT1, 38.1, HIT3a, RFT8, SK7, BC3, SP34-2, HU291, TRX4, Catumaxomab, teplizumab, 3-106, 3-114, 3-148, 3-190, 3-271, 3-550, 4-10, 4-48, H2C, F12Q, I2C, SP7, 3F3A1, CD3-12, 301, RIV9, JB38-29, JE17-74, GT0013, 4E2, 7A4, 4D10A6, SPV-T3b, M2AB, ICO-90, 30A1 or Hu38E4.v1 (US patent application 20200299409A1), REGN5458 (US patent application 20200024356A1), Blinatumomab (https://go.drugbank.com/drugs/DB09052/polypeptide sequences fasta). In some embodiments, the conjugate comprises a Fab, wherein the Fab comprises (a) a heavy chain fragment comprising the amino acid sequence of SEQ ID NO: 1 and a light chain fragment comprising the amino acid sequence of SEQ ID NO: 2 or 3.

An exemplary CD4 binding agent can be an antibody selected from the group consisting of Ibalizumab (https://www.genome.jp/dbget-bin/www_bget?D09575), AF1856 (R&D Systems), MAB554 (R&D Systems), BF0174 (Affinity Biosciences), PAB31115 (Abnova Corporation), CAL4 (Abcam), and antigen binding fragments thereof. In certain embodiments, the binding agent comprises a V_{H} domain and a V_{L} domain of an antibody selected from the group consisting of AF1856 (R&D Systems), MAB554 (R&D Systems), BF0174 (Affinity Biosciences), PAB31115 (Abnova Corporation), and CAL4 (Abcam). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL. BIOL. 262:732-745), or any other CDR determination method known in the art, of the V_{H} and V_{L} sequences of an antibody selected from the group consisting of AF1856 (R&D Systems), MAB554 (R&D Systems), BF0174 (Affinity Biosciences), PAB31115 (Abnova Corporation), and CAL4 (Abcam).

An exemplary CD4 binding agent can also be selected from antibodies or antibody fragments employing CDRs of clones Ibalizumab, OKT4, RPA-T4, S3.5, SK3, NIUGO, RIV6, OTI18E3, MEM-241, B486A1, RFT-4g, 7E14, MDX.2, MEM-115, MEM-16, ICO-86, Edu-2, or ilbalizumab.

An exemplary CDS binding agent can be an antibody selected from the group consisting of He3, MAB1636 (R&D Systems), AF1636 (R&D Systems), MAB115 (R&D Systems), C5/473+CD5/54/F6 (Abcam), CD5/54/F6 (Abcam), 65152 (Proteintech), and antigen binding fragments thereof. In some embodiments, the binding agent comprises a V_{H} domain and a V_{L} of an antibody selected from the group consisting of MAB1636 (R&D Systems), AF1636 (R&D Systems), MAB115 (R&D Systems), C5/473+CD5/54/F6 (Abcam), CD5/54/F6 (Abcam), and 65152 (Proteintech). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL. BIOL. 262:732-745), or any other CDR determination method known in the art, of the V_{H} and V_{L} sequences of an antibody selected from the group consisting of MAB1636 (R&D Systems), AF1636 (R&D Systems), MAB115 (R&D Systems), C5/473+CD5/54/F6 (Abcam), CD5/54/F6 (Abcam), and 65152 (Proteintech).

An exemplary CDS binding agent can also be selected from antibodies or antibody fragments employing CDRs of clones of zolimomab, 5D7, L17F12, and UCHT2, 1D8, 3121, 4H10, 8J23, 504, 4H2, 5G2, 8G8, 6M4, 2E3, 4E24, 4F10, 7J9, 7P9, 8E24, 6L18, 7H7, 1E7, 8J21, 7111, 8M9, 1P21, 2H11, 3M22, 5M6, 5H8, 7119, 1A2, 8E15, 8C10, 3P16, 4F3, 5M24, 5024, 7B16, 1E8, 2H16, BLal, 1804, DK23, Cris1, MEM-32, H65, 4C7, OX-19, Leu-1, 53-7.3, 4H8E6, T101, EP2952, D-9, H-3, HK231, N-20, Y2/178, H-300, CD5/54/F6, Q-20, CC17, MOM-18539-S(P), or MOM-18885-S(P).

An exemplary CD7 binding agent can be an antibody selected from the group consisting of MAB7579 (R&D Systems), AF7579 (R&D Systems), EPR22065 (Abcam), 1G10D8 (Proteintech), NBP2-32097 (Novus Biologicals), NBP2-38440 (Novus Biologicals), and antigen binding fragments thereof. In certain embodiments, the binding agent comprises a VH domain and a VL of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL. BIOL. 262:732-745), or any other CDR determination method known in the art, of the V_{H} and V_{L} sequences of an antibody selected from the group consisting of MAB7579 (R&D Systems), AF7579 (R&D Systems), EPR22065 (Abcam), 1G10D8 (Proteintech), NBP2-32097 (Novus Biologicals), and NBP2-38440 (Novus Biologicals).

An exemplary CD7 binding agent can also be selected from antibodies or antibody fragments employing CDRs of clones TH-69, 3Afl1, T3-3A1, 124-1D1, 3Alf, CD7-6B7, or VHH6.

An exemplary CD8 (CD8α, CD8α/α, CD8α/β or CD8β) binding agent can be an antibody selected from the group consisting of 2.43 (Invitrogen), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), YTS105.18 (CD8α, Novus Biologicals), TRX2 (https://patents.justia.com/patent/20170198045), and antigen binding fragments thereof. In certain embodiments, the binding agent comprises a V_{H} domain and a V_{L} domain of an antibody selected from the group consisting of 2.43 (Invitrogen), 51.1 (ATCC HB-230), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), and YTS105.18 (CD8α, Novus Biologicals). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL. BIOL. 262:732-745), or any other CDR determination method known in the art, of the V_{H} and V_{L} sequences of an antibody selected from the group consisting of 2.43 (Invitrogen), Du CD8-1 (CD8α, Invitrogen), 9358-CD (CD8α/β, R&D Systems), MAB116 (CD8α, R&D Systems), ab4055 (CD8α, Abcam), C8/144B (CD8α, Novus Biologicals), and YTS105.18 (CD8α, Novus Biologicals).

An exemplary CD8 binding agent can also be selected from antibodies or antibody fragments employing CDRs of clones OKT-8, 51.1, S6F1, TRX2, and UCHT4, SP16, 3B5, C8-144B, HIT8a, RAVB3, LT8, 17D8, MEM-31, MEM-87, RIV11, DK-25, YTC141.1HL, or YTC182.20. In some embodiments, the conjugate comprises a Fab, wherein the Fab comprises a heavy chain fragment comprising the amino acid sequence of SEQ ID NO: 6 and a light chain fragment comprising the amino acid sequence of SEQ ID NO: 7.

An exemplary CD137 binding agent can be selected from antibodies or antibody fragments employing CDRs of clones 4B4-1, P566, or Urelumab. An exemplary CD28 binding agent can be selected from antibodies or antibody fragments employing CDRs of clone TAB08. An exemplary CD45 binding agent can be selected from antibodies or antibody fragments employing CDRs of clones BC8, 9.4, 4B2, Tu116, or GAP8.3. An exemplary CD18 binding agent can be selected from antibodies or antibody fragments employing CDRs of clones 1B4, TS1/18, MEM-48, YFC118-3, TA-4, MEM-148, or R³-3, 24. An exemplary CD11a binding agent can be selected from antibodies or antibody fragments employing CDRs of clone MHM24 or Efalizumab. An exemplary IL-2 receptor binding agent can be selected from of antibodies or antibody fragments employing CDRs of clones YTH 906.9HL, IL2R.1, BC96, B-B10, 216, MEM-181, ITYV, MEM-140, ICO-105, Daclizumab, or from the group consisting of IL2 or fragments of IL2. An exemplary IL-15R binding agent can be selected from antibodies or antibody fragments employing CDRs of clones JM7A4, or OTI3D5, or from the group consisting of IL15 or fragments of IL15. An exemplary TLR2 binding agent can be selected from antibodies or antibody fragments employing CDRs of clones JM22-41, TL2.1, 11G7, or TLR2.45. An exemplary TLR4 binding agent can be selected from antibodies or antibody fragments employing CDRs of clones HTA125, or 76B357-1. An exemplary TLR5 binding agent can be selected from antibodies or antibody fragments employing CDRs of clones 85B152-5, or 9D759-2. An exemplary GL7 binding agent can be selected from antibodies or antibody fragments employing CDRs of clone GL7.

An exemplary PD1 binding agent can be selected from antibodies or antibody fragments employing CDRs of clones MIH4, J116, J150, OTIB11, OTI17B10, OTI3A1, or OTI16D4. In addition, exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, for example, atezolizumab (Tecentriq®, Genentech), durvalumab (AstraZeneca), MEDI4736, avelumab, and BMS 936559 (Bristol Myers Squibb Co.).

An exemplary CTLA-4 binding agent can be selected from antibodies or antibody fragments employing CDRs of clones ER4.7G.11 [7G11], OTI9G4, OTI9F3, OTI3A5, A3.4H2.H12, 14D3, OTI3A12, OTI1A11, OTI1E8, OTI3B11, OTI3D2, OTI10C8, OTI2E9, OTI6F1, OTI7D3, OTI85B, OTI12C6. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

An exemplary TCR β binding agent can be an antibody selected from the group consisting of H57-597 (Invitrogen), 8A3 (Novus Biologicals), R73 (TCRα/β, Abcam), E6Z3S (TRBC1/TCRβ, Cell Signaling Technology), and antigen binding fragments thereof. In certain embodiments, the binding agent comprises a V_{H} domain and a V_{L} of an antibody selected from the group consisting of H57-597 (Invitrogen), 8A3 (Novus Biologicals), R73 (TCRα/β, Abcam), and E6Z3S (TRBC1/TCRβ, Cell Signaling Technology). In certain embodiments, the binding agent comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Chothia C & Lesk A M, (1987), J. MOL. BIOL. 196:901-917), MacCallum (see, MacCallum R M et al., (1996) J. MOL. BIOL. 262:732-745), or any other CDR determination method known in the art, of the V_{H} and V_{L} sequences of an antibody selected from the group consisting of H57-597 (Invitrogen), 8A3 (Novus Biologicals), R73 (TCRα/β, Abcam), and E6Z3S (TRBC1/TCRβ, Cell Signaling Technology).

An exemplary CD137 binding agent can be selected from antibodies or antibody fragments employing CDRs of clones 4B4-1, P566, or Urelumab.

In some embodiments, the cell targeting group comprises an antibody selected from the group consisting of a Fab, F(ab′)2, Fab′-SH, Fv, and scFv fragment. In some embodiments, the antibody is a human or humanized antibody. In some embodiments, the cell targeting group comprises a Fab or an immunoglobulin single variable domain, such as a Nanobody. In some embodiments, the cell targeting group comprises a Fab that does not comprise a natural interchain disulfide bond. For example, in some embodiments, the Fab comprises a heavy chain fragment that comprises a C233S substitution, and/or a light chain fragment that comprises a C214S substitution, numbering according to Kabat. In some embodiments, the cell targeting group comprises a Fab that comprises one or more non-native interchain disulfide bonds. In some embodiments, the interchain disulfide bonds are between two non-native cysteine residues on the light chain fragment and heavy chain fragment, respectively. For example, in some embodiments, the Fab comprises a heavy chain fragment that comprises F174C substitution, and/or a light chain fragment that comprises S176C substitution, numbering according to Kabat. In some embodiments, the Fab comprises a heavy chain fragment that comprises F174C and C233S substitutions, and/or a light chain fragment that comprises S176C and C214S substitutions, numbering according to Kabat. In some embodiments, the cell targeting group comprises a C-terminal cysteine residue. In some embodiments, the cell targeting group comprises a Fab that comprises a cysteine at the C-terminus of the heavy or light chain fragment. In some embodiments, the Fab further comprises one or more amino acids between the heavy chain of the Fab and the C-terminal cysteine. For example, in some embodiments, the Fab comprises two or more amino acids derived from an antibody hinge region (e.g., a partial hinge sequence) between the C-terminus of the Fab and the C-terminal cysteine. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the cell targeting group further comprises a single chain variable fragment (scFv) linked to the C-terminus of the light chain constant domain by an amino acid linker. In some embodiments, the Fab antibody is a DS Fab, a NoDS Fab, a bDS Fab, a bDS Fab-ScFv.

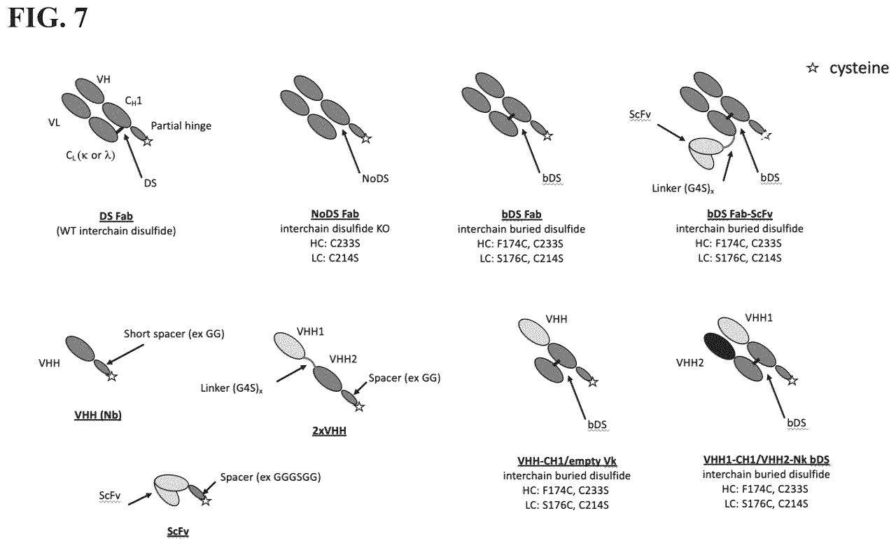

In some embodiments, the cell targeting group comprises an immunoglobulin single variable domain, such as a Nanobody (e.g., a V_{HH}). In some embodiments, the Nanobody comprises a cysteine at the C-terminus. In some embodiments, the Nanobody further comprises a spacer comprising one or more amino acids between the V_{HH} domain and the C-terminal cysteine. In some embodiments, the spacer comprises one or more glycine residues, e.g., two glycine residues. In some embodiments, the cell targeting group comprises two or more V_{HH} domains. In some embodiments, the two or more V_{HH} domains are linked by an amino acid linker. In some embodiments, the amino acid linker comprises one or more glycine and/or serine residues (e.g., one or more repeats of the sequence GGGGS). In some embodiments, the cell targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds (e.g., interchain disulfide bonds). In some embodiments, the cell targeting group comprises a V_{HH} domain linked to an antibody CH1 domain, and wherein the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat. In some embodiments, the antibody is a ScFv, a V_{HH}, a 2×V_{HH}, a VAR-CH1/empty Vk, or a V_{HH}1-CH1/V_{HH}-2-Nb bDS, as demonstrated in FIG. 7.

An exemplary targeting moiety may have an amino sequence as set forth below:

Anti-CD3 hSP34-Fab sequences:
hSP34 heavy chain (HC) sequence (SEQ ID NO: 1):
EVQLVESGGGLVQPGGSLKLSCAASGFTFNKYAMNWVRQAPGKGLEWVARIRSKYNN
YATYYADSVKDRFTISRDDSKNTAYLQMNNLKTEDTAVYYCVRHGNFGNSYISYWAY
WGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSSDKTHT
C
hSP34-mlam light chain (LC) sequence (mouse lambda) (SEQ ID NO: 2):
QTVVTQEPSLTVSPGGTVTLTCGSSTGAVTSGNYPNWVQQKPGQAPRGLIGGTKFLAPG
TPARFSGSLLGGKAALTLSGVQPEDEAEYYCVLWYSNRWVFGGGTKLTVLGQPKSSPS
VTLFPPSSEELETNKATLVCTITDFYPGVVTVDWKVDGTPVTQGMETTQPSKQSNNKY
MASSYLTLTARAWERHSSYSCQVTHEGHTVEKSLSRADSS
SP34-hlam LC (human lambda) (SEQ ID NO: 3):
QTVVTQEPSLTVSPGGTVTLTCGSSTGAVTSGNYPNWVQQKPGQAPRGLIGGTKFLAPG
TPARFSGSLLGGKAALTLSGVQPEDEAEYYCVLWYSNRWVFGGGTKLTVLSQPKAAPS
VTLFPPSSEELQANKATLVCLVSDFYPGAVTVAWKADGSPVKVGVETTKPSKQSNNKY
AASSYLSLTPEQWKSHRSYSCRVTHEGSTVEKTVAPAESS
Anti-CD3 Hu291-Fab sequences:
Hu291 HC (SEQ ID NO: 4):
QVQLVQSGAEVKKPGASVKVSCKASGYTFISYTMHWVRQAPGQGLEWMGYINPRSGY
THYNQKLKDKATLTADKSASTAYMELSSLRSEDTAVYYCARSAYYDYDGFAYWGQGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSSDKTHTC
Hu 291 LC (SEQ ID NO: 5):
MDMRVPAQLLGLLLLWLPGAKCDIQMTQSPSSLSASVGDRVTITCSASSSVSYMNWYQ
QKPGKAPKRLIYDTSKLASGVPSRFSGSGSGTDFTLTISSLQPEDFATYYCQQWSSNPPTF
GGGTKVEIKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGES
Anti-CD8 TRX2-Fab sequences:
TRX2 HC (SEQ ID NO: 6):
QVQLVESGGGVVQPGRSLRLSCAASGFTFSDFGMNWVRQAPGKGLEWVALIYYDGSN
KFYADSVKGRFTISRDNSKNTLYLQMNSLRAEDTAVYYCAKPHYDGYYHFFDSWGQG
TLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSSDKTHTC
TRX2 LC (SEQ ID NO: 7):
DIQMTQSPSSLSASVGDRVTITCKGSQDINNYLAWYQQKPGKAPKLLIYNTDILHTGVPS
RFSGSGSGTDFTFTISSLQPEDIATYYCYQYNNGYTFGQGTKVEIKRTVAAPSVFIFPPSD
EQLKSGTASVVCLLNNFYPREAKVQWKVDNALQSGNSQESVTEQDSKDSTYSLSSTLTL
SKADYEKHKVYACEVTHQGLSSPVTKSFNRGES
Anti-CD8 OKT8-Fab sequences:
OKT8 HC (SEQ ID NO: 8):
QVQLVQSGAEDKKPGASVKVSCKASGFNIKDTYIHWVRQAPGQGLEWMGRIDPANDN
TLYASKFQGRVTITADTSSNTAYMELSSLRSEDTAVYYCGRGYGYYVFDHWGQGTTVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSSDKTHTC
OKT8 LC (SEQ ID NO: 9):
DIVMTQSPSSLSASVGDRVTITCRTSRSISQYLAWYQEKPGKAPKLLIYSGSTLQSGVPSR
FSGSGSGTDFTLTISSLQPEDFATYYCQQHNENPLTFGQGTKVEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGES
Anti-CD4 Ibalizumab-Fab sequences:
Ibalizumab HC (SEQ ID NO: 10):
QVQLQQSGPEVVKPGASVKMSCKASGYTFTSYVIHWVRQKPGQGLDWIGYINPYNDGT
DYDEKFKGKATLTSDTSTSTAYMELSSLRSEDTAVYYCAREKDNYATGAWFAYWGQG
TLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSSDKTHTC
Ibalizumab LC (SEQ ID NO: 11):
DIVMTQSPDSLAVSLGERVTMNCKSSQSLLYSTNQKNYLAWYQQKPGQSPKLLIYWAS
TRESGVPDRFSGSGSGTDFTLTISSVQAEDVAVYYCQQYYSYRTFGGGTKLEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGES
anti-CDS He3-Fab sequences:
He3 HC (SEQ ID NO: 12):
EIQLVQSGGGL VKPGGSVRISCAASGYTFTNYGMNWVRQAPGKGLEWMGWINTHTGEP
TYADSFKGRFTFSLDDSKNTAYLQINSLRAEDTAVYFCTRRGYDWYFDVWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSSDKTHTC
He3 LC (SEQ ID NO: 13):
DIQMTQSPSSLSASVGDRVTITCRASQDINSYLSWFQQKPGKAPKTLIYRANRLESGVPSR
FSGSGSGTDYTLTISSLQYEDFGIYYCQQYDESPWTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKFNRGES
anti-CD7 TH-69-Fab sequences:
TH-69 HC (SEQ ID NO: 14):
EVQLVESGGGLVKPGGSLKLSCAASGLTFSSYAMSWVRQTPEKRLEWVASISSGGFTYY
PDSVKGRFTISRDNARNILYLQMSSLRSEDTAMYYCARDEVRGYLDVWGAGTTVTVSS
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTC
TH-69 LC (SEQ ID NO: 15):
DIQMTQTTSSLSASLGDRVTISCSASQGISNYLNWYQQKPDGTVKLLIYYTSSLHSGVPSR
FSGSGSGTDYSLTISNLEPEDIATYYCQQYSKLPYTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
anti-CD2 TS2/18.1-Fab sequences:
TS2/18.1 HC (SEQ ID NO: 16):
EVQLVESGGGLVMPGGSLKLSCAASGFAFSSYDMSWVRQTPEKRLEWVAYISGGGFTY
YPDTVKGRFTLSRDNAKNTLYLQMSSLKSEDTAMYYCARQGANWELVYWGQGTLVTV
SAASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSSDKTHTC
TS2/18.1 LC (SEQ ID NO: 17):
DIVMTQSPATLSVTPGDRVFLSCRASQSISDFLHWYQQKSHESPRLLIKYASQSISGIPSRF
SGSGSGSDFTLSINSVEPEDVGVYFCQNGHNFPPTFGGGTKLEIKRTVAAPSVFIFPPSDEQ
LKSGTASVVCLLNNFYPREAKVQWKVDNALQSGNSQESVTEQDSKDSTYSLSSTLTLSK
ADYEKHKVYACEVTHQGLSSPVTKSFNRGES
anti-CD2 9.6-Fab sequences:
9.6 HC (SEQ ID NO: 18):
QVQLQQPGAELVRPGSSVKLSCKASGYTFTRYWIHWVKQRPIQGLEWIGNIDPSDSETH
YNQKFKDKATLTVDKSSGTAYMQLSSLTSEDSAVYYCATEDLYYAMEYWGQGTSVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSSDKTHTC
9.6 LC (SEQ ID NO: 19):
NIMMTQSPSSLAVSAGEKVTMTCKSSQSVLYSSNQKNYLAWYQQKPGQSPKLLIYWAS
TRESGVPDRFTGSGSGTDFTLTISSVQPEDLAVYYCHQYLSSHTFGGGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGES
anti-CD2 9-1-Fab sequences:
9-1 HC (SEQ ID NO: 20):
QVQLQQPGTELVRPGSSVKLSCKASGYTFTSYWVNWVKQRPDQGLEWIGRIDPYDSETH
YNQKFTDKAISTIDTSSNTAYMQLSTLTSDASAVYYCSRSPRDSSTNLADWGQGTLVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSSDKTHTC
9-1 LC (SEQ ID NO: 21):
DIVMTQSPATLSVTPGDRVSLSCRASQSISDYLHWYQQKSHESPRLLIKYASQSISGIPSRF
SGSGSGSDFTLSINSVEPEDVGVYYCQNGHSFPLTFGAGTKLELRRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGES
mutOKT8-Fab sequences:
mutOKT8 HC (SEQ ID NO: 22):
QVQLVQSGAEDKKPGASVKVSCKASGFNIKDTYIHWVRQAPGQGLEWMGRIDPANDNT
LYASKFQGRVTITADTSSNTAYMELSSLRSEDTAVYYCGRGAGAYVFDHWGQGTTVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSSDKTHTC
mutOKT8 LC (SEQ ID NO: 23):
DIVMTQSPSSLSASVGDRVTITCRTSRSISAALAWYQEKPGKAPKLLIYSGSTLQSGVPSR
FSGSGSGTDFTLTISSLQPEDFATYYCQQHNENPLTFGQGTKVEIKRTVAAPSVFIFPPSDE
QLKSGTASVVCLLNNFYPREAKVQWKVDNALQSGNSQESVTEQDSKDSTYSLSSTLILS
KADYEKHKVYACEVTHQGLSSPVTKSFNRGES.
Anti-CD56 A1 Fab sequence
A1 bDS HC (SEQ ID NO: 26):
QVQLQQSGPGLVKPSQTLSLTCAISGDSVSSNSAAWNWIRQSPSNWIRQSPSGLEWLGR
TYYRSKWYNDYAVSVKSRITINPDTSKNQFSLQLNSVTPEDTAVYYCARENIAAWTWA
FDIWGQGTMVTVSSASTKGPSVFPLAPSSKSTSGGTAALGCLVKDYFPEPVTVSWNSGA
LTSGVHTCPAVLQSSGLYSLSSVVTVPSSSLGTQTYICNVNHKPSNTKVDKKVEPKSSDK
THTCGGHHHHHH
A1 bDS LC (SEQ ID NO: 27):
EIVMTQSPGTLSLSPGERATLSCRASQSVSSSYLAWYQQKPGLAPRLLIYDTSLRATDIPD
RFSGSGSGTAFTLTISRLEPEDFAVYYCQQYGSSPTFGQGTKVEIKRTVAAPSVFIFPPSDE
QLKSGTASVVCLLNNFYPREAKVQWKVDNALQSGNSQESVTEQDSKDSTYSLCSTLTL
SKADYEKHKVYACEVTHQGLSSPVTKSFNRGES
Anti-CD56 A2 Fab sequence
A2 bDS HC (SEQ ID NO: 28):
EVQLVQSGAEVKKPGSSVKVSCKASGGTFTGYYMHWVRQAPGQGLEWMGWINPNSG
GTNYAQKFQGRVTMTRDTSISTAYMELSRLRSDDTAVYYCARDLSSGYSGYFDYWGQ
GTLVTVSSASTKGPSVFPLAPSSKSTSGGTAALGCLVKDYFPEPVTVSWNSGALTSGVHT
CPAVLQSSGLYSLSSVVTVPSSSLGTQTYICNVNHKPSNTKVDKKVEPKSSDKTHTCGG
HHHHHH
A2 bDS LC (SEQ ID NO: 29):
DVVMTQSPLSLPVTPGEPASISCRSSQSLLHSNGYNYLNWYLQKPGQSPQLLIYLGSNRA
SGVPDRFSGSGSGTDFTLKISRVEGEDVGDYYCMQALQSPFTFGQGTKLEIKRTVAAPSV
FIFPPSDEQLKSGTASVVCLLNNFYPREAKVQWKVDNALQSGNSQESVTEQDSKDSTYS
LCSTLTLSKADYEKHKVYACEVTHQGLSSPVTKSFNRGES
Anti-CD56 A3 Fab sequence
A3 bDS HC (SEQ ID NO: 30):
EVQLVQSGAEVKKPGSSVKVSCKASGGTFTGYYMHWVRQAPGQGLEWMGWINPNSG
GTNYAQKFQGRVTMTRDTSISTAYMELSRLRSDDTAVYYCARDLSSGYSGYFDYWGQ
GTLVTVSSASTKGPSVFPLAPSSKSTSGGTAALGCLVKDYFPEPVTVSWNSGALTSGVHT
CPAVLQSSGLYSLSSVVTVPSSSLGTQTYICNVNHKPSNTKVDKKVEPKSSDKTHTCGG
HHHHHH
A3 bDS LC (SEQ ID NO: 31):
DVVMTQSPLSLPVTPGEPASISCRSSQSLLHSNGYNFLDWYLQKPGQSPQLLIYLGSNRA
SGVPDRFSGSGSGTDFTLKISRVEADDVGVYYCMQSLQTPWTFGHGTKVEIKRTVAAPS
VFIFPPSDEQLKSGTASVVCLLNNFYPREAKVQWKVDNALQSGNSQESVTEQDSKDSTY
SLCSTLTLSKADYEKHKVYACEVTHQGLSSPVTKSFNRGES
Anti-CD56 Lorvotuzumab Fab sequence
Lorvotuzumab bDS HC (SEQ ID NO: 32):
QVQLVESGGG VVQPGRSLRL SCAASGFTFS SFGMHWVRQA
PGKGLEWVAYISSGSFTIYY ADSVKGRFTI SRDNSKNTLY LQMNSLRAED
TAVYYCARMR KGYAMDYWGQ
GTLVTVSSASTKGPSVFPLAPSSKSTSGGTAALGCLVKDYFPEPVTVSWNSGALTSGVHT
CPAVLQSSGLYSLSSVVTVPSSSLGTQTYICNVNHKPSNTKVDKKVEPKSSDKTHTCHH
HHHH
Lorvotuzumab bDS LC (SEQ ID NO: 33):
DVVMTQSPLSLPVTLGQPASISCRSSQIIIHSDGNTYLEWFQQRPGQSPRRLIYKVSNRFS
GVPDRFSGSGSGTDFTLKISRVEAEDVGVYYCFQGSHVPHTFGQGTKVEIKRTVAAPSV
FIFPPSDEQLKSGTASVVCLLNNFYPREAKVQWKVDNALQSGNSQESVTEQDSKDSTYS
LCSTLTLSKADYEKHKVYACEVTHQGLSSPVTKSFNRGES
Anti-CD2 RPA-2.10v1 Fab sequence
RPA-2.10v1 bDS HC (SEQ ID NO: 34):
EVKLVESGGGLVKPGGSLKLSCAASGFTFSSYDMSWVRQTPEKRLEWVASISGGGFLY
YLDSVKGRFTISRDNARNILYLHMTSLRSEDTAMYYCARSSYGEIMDYWGQGTSVTVSS
ASTKGPSVFPLAPSSKSTSGGTAALGCLVKDYFPEPVTVSWNSGALTSGVHTCPAVLQSS
GLYSLSSVVTVPSSSLGTQTYICNVNHKPSNTKVDKKVEPKSSDKTHTCHHHHHH
RPA-2.10v1 bDS LC (SEQ ID NO: 35):
DILLTQSPAILSVSPGERVSFSCRASQRIGTSIHWYQQRTTGSPRLLIKYASESISGIPSRFSG
SGSGTDFTLSINSVESEDVADYYCQQSHGWPFTFGGGTKLEIERTVAAPSVFIFPPSDEQL
KSGTASVVCLLNNFYPREAKVQWKVDNALQSGNSQESVTEQDSKDSTYSLCSTLTLSK
ADYEKHKVYACEVTHQGLSSPVTKSFNRGES
Anti-CD137 4B4-1 Fab sequence
4B4-1 bDS HC (SEQ ID NO: 36):
QVQLQQPGAELVKPGASVKLSCKASGYTFSSYWMHWVKQRPGQVLEWIGEINPGNGH
TNYNEKFKSKATLTVDKSSSTAYMQLSSLTSEDSAVYYCARSFTTARGFAYWGQGTLV
TVSASTKGPSVFPLAPSSKSTSGGTAALGCLVKDYFPEPVTVSWNSGALTSGVHTCPAV
LQSSGLYSLSSVVTVPSSSLGTQTYICNVNHKPSNTKVDKKVEPKSSDKTHTCHHHHHH
4B4-1 bDS LC (SEQ ID NO: 37):
DIVMTQSPATQSVTPGDRVSLSCRASQTISDYLHWYQQKSHESPRLLIKYASQSISGIPSR
FSGSGSGSDFTLSINSVEPEDVGVYYCQDGHSFPPTFGGGTKLEIKRTVAAPSVFIFPPSDE
QLKSGTASVVCLLNNFYPREAKVQWKVDNALQSGNSQESVTEQDSKDSTYSLCSTLTL
SKADYEKHKVYACEVTHQGLSSPVTKSFNRGES
hSP34-hlam NoDS HC (SEQ ID NO: 38):
EVQLVESGGGLVQPGGSLKLSCAASGFTFNKYAMNWVRQAPGKGLEWVARIRSKYNN
YATYYADSVKDRFTISRDDSKNTAYLQMNNLKTEDTAVYYCVRHGNFGNSYISYWAY
WGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSSDKTHT
C
hSP34-hlam NoDS LC (SEQ ID NO: 39):
QTVVTQEPSLTVSPGGTVTLTCGSSTGAVTSGNYPNWVQQKPGQAPRGLIGGTKFLAPG
TPARFSGSLLGGKAALTLSGVQPEDEAEYYCVLWYSNRWVFGGGTKLTVLSQPKAAPS
VTLFPPSSEELQANKATLVCLVSDFYPGAVTVAWKADGSPVKVGVETTKPSKQSNNKY
AASSYLSLTPEQWKSHRSYSCRVTHEGSTVEKTVAPAESS
hSP34-hlam DS HC (SEQ ID NO: 40):
EVQLVESGGGLVQPGGSLKLSCAASGFTFNKYAMNWVRQAPGKGLEWVARIRSKYNN
YATYYADSVKDRFTISRDDSKNTAYLQMNNLKTEDTAVYYCVRHGNFGNSYISYWAY
WGQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTH
TC
hSP34-hlam DS LC (SEQ ID NO: 41):
QTVVTQEPSLTVSPGGTVTLTCGSSTGAVTSGNYPNWVQQKPGQAPRGLIGGTKFLAPG
TPARFSGSLLGGKAALTLSGVQPEDEAEYYCVLWYSNRWVFGGGTKLTVLSQPKAAPS
VTLFPPSSEELQANKATLVCLVSDFYPGAVTVAWKADGSPVKVGVETTKPSKQSNNKY
AASSYLSLTPEQWKSHRSYSCRVTHEGSTVEKTVAPAECS
Anti-CD2 TS2/18.1 DS Fab
TS2/18.1 DS HC (SEQ ID NO: 42):
EVQLVESGGGLVMPGGSLKLSCAASGFAFSSYDMSWVRQTPEKRLEWVAYISGGGFTY
YPDTVKGRFTLSRDNAKNTLYLQMSSLKSEDTAMYYCARQGANWELVYWGQGTLVT
VSAASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTC
TS2/18.1 DS LC (SEQ ID NO: 43):
DIVMTQSPATLSVTPGDRVFLSCRASQSISDFLHWYQQKSHESPRLLIKYASQSISGIPSRF
SGSGSGSDFTLSINSVEPEDVGVYFCQNGHNFPPTFGGGTKLEIKRTVAAPSVFIFPPSDE
QLKSGTASVVCLLNNFYPREAKVQWKVDNALQSGNSQESVTEQDSKDSTYSLSSTLTLS
KADYEKHKVYACEVTHQGLSSPVTKSFNRGEC
Anti-CD2 9.6 DS Fab
9.6 DS HC (SEQ ID NO: 44):
QVQLQQPGAELVRPGSSVKLSCKASGYTFTRYWIHWVKQRPIQGLEWIGNIDPSDSETH
YNQKFKDKATLTVDKSSGTAYMQLSSLTSEDSAVYYCATEDLYYAMEYWGQGTSVTV
SSASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTC
9.6 DS LC (SEQ ID NO: 45):
NIMMTQSPSSLAVSAGEKVTMTCKSSQSVLYSSNQKNYLAWYQQKPGQSPKLLIYWAS
TRESGVPDRFTGSGSGTDFTLTISSVQPEDLAVYYCHQYLSSHTFGGGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
hSP34-hlam bDS HC (SEQ ID NO: 46):
EVQLVESGGGLVQPGGSLKLSCAASGFTFNKYAMNWVRQAPGKGLEWVARIRSKYNN
YATYYADSVKDRFTISRDDSKNTAYLQMNNLKTEDTAVYYCVRHGNFGNSYISYWAY
WGQGTLVTVSSASTKGPSVFPLAPSSKSTSGGTAALGCLVKDYFPEPVTVSWNSGALTS
GVHTCPAVLQSSGLYSLSSVVTVPSSSLGTQTYICNVNHKPSNTKVDKKVEPKSSDKTH
TCHHHHHH
hSP34-hlam bDS LC (SEQ ID NO: 47):
QTVVTQEPSLTVSPGGTVTLTCGSSTGAVTSGNYPNWVQQKPGQAPRGLIGGTKFLAPG
TPARFSGSLLGGKAALTLSGVQPEDEAEYYCVLWYSNRWVFGGGTKLTVLSQPKAAPS
VTLFPPSSEELQANKATLVCLVSDFYPGAVTVAWKADGSPVKVGVETTKPSKQSNNKY
AACSYLSLTPEQWKSHRSYSCRVTHEGSTVEKTVAPAESS
Anti-CD3 TR66 bDS Fab sequence
TR66 bDS HC (SEQ ID NO: 48):
QVQLQQSGAELARPGASVKMSCKTSGYTFTRYTMHWVKQRPGQGLEWIGYINPSRGY
TNYNQKFKDKATLTTDKSSSTAYMQLSSLTSEDSAVYYCARYYDDNYSLDYWGQGTT
LTVSSASTKGPSVFPLAPSSKSTSGGTAALGCLVKDYFPEPVTVSWNSGALTSGVHTCPA
VLQSSGLYSLSSVVTVPSSSLGTQTYICNVNHKPSNTKVDKKVEPKSSDKTHTCHHHHH
H
TR66 bDS LC (SEQ ID NO: 49):
QIVLTQSPSSLSASLGEKVTMTCRASSSVSYMNWYQQKPGTSPKRWIYDTSKVASGVPD
RFSGSGSGTSYSLTISSMEAEDAATYYCQQWSSNPLTFGAGTKLELKRTVAAPSVFIFPPS
DEQLKSGTASVVCLLNNFYPREAKVQWKVDNALQSGNSQESVTEQDSKDSTYSLCSTL
TLSKADYEKHKVYACEVTHQGLSSPVTKSFNRGES
Anti-CD3 TRX4 bDS Fab sequence
TRX4 bDS HC (SEQ ID NO: 50):
EVQLLESGGGLVQPGGSLRLSCAASGFTFSSFPMAWVRQAPGKGLEWVSTISTSGGRTY
YRDSVKGRFTISRDNSKNTLYLQMNSLRAEDTAVYYCAKFRQYSGGFDYWGQGTLVT
VSSASTKGPSVFPLAPSSKSTSGGTAALGCLVKDYFPEPVTVSWNSGALTSGVHTCPAVL
QSSGLYSLSSVVTVPSSSLGTQTYICNVNHKPSNTKVDKKVEPKSSDKTHTCHHHHHH
TRX4 bDS LC (SEQ ID NO: 51):
DIQLTQPNSVSTSLGSTVKLSCTLSSGNIENNYVHWYQLYEGRSPTTMIYDDDKRPDGVP
DRFSGSIDRSSNSAFLTIHNVAIEDEAIYFCHSYVSSFNVFGGGTKLTVLGQPKANPTVTL
FPPSSEELQANKATLVCLISDFYPGAVTVAWKADGSPVKAGVETTKPSKQSNNKYAACS
YLSLTPEQWKSHRSYSCQVTHEGSTVEKTVAPTESS
Anti-CD3 HzUCHT1 bDS Fab sequence
HzUCHT1(Y59T) bDS HC (SEQ ID NO: 52):
EVQLVESGGGLVQPGGSLRLSCAASGYSFTGYTMNWVRQAPGKGLEWVALINPTKGVS
TYNQKFKDRFTISVDKSKNTAYLQMNSLRAEDTAVYYCARSGYYGDSDWYFDVWGQ
GTLVTVSSASTKGPSVFPLAPSSKSTSGGTAALGCLVKDYFPEPVTVSWNSGALTSGVHT
CPAVLQSSGLYSLSSVVTVPSSSLGTQTYICNVNHKPSNTKVDKKVEPKSSDKTHTCHH
HHHH
HzUCHT1 bDS LC (SEQ ID NO: 53):
DIQMTQSPSSLSASVGDRVTITCRASQDIRNYLNWYQQKPGKAPKLLIYYTSRLESGVPS
RFSGSGSGTDYTLTISSLQPEDFATYYCQQGNTLPWTFGQGTKVEIKRTVAAPSVFIFPPS
DEQLKSGTASVVCLLNNFYPREAKVQWKVDNALQSGNSQESVTEQDSKDSTYSLCSTL
TLSKADYEKHKVYACEVTHQGLSSPVTKSFNRGES
Anti-CD3 Teplizumab bDS Fab sequence
Teplizumab bDS HC (SEQ ID NO: 54):
QVQLVQSGGGVVQPGRSLRLSCKASGYTFTRYTMHWVRQAPGKGLEWIGYINPSRGYT
NYNQKVKDRFTISRDNSKNTAFLQMDSLRPEDTGVYFCARYYDDHYCLDYWGQGTPV
TVSSASTKGPSVFPLAPSSKSTSGGTAALGCLVKDYFPEPVTVSWNSGALTSGVHTCPAV
LQSSGLYSLSSVVTVPSSSLGTQTYICNVNHKPSNTKVDKKVEPKSSDKTHTCHHHHHH
Teplizumab bDS LC (SEQ ID NO: 55):
DIQMTQSPSSLSASVGDRVTITCSASSSVSYMNWYQQTPGKAPKRWIYDTSKLASGVPS
RFSGSGSGTDYTFTISSLQPEDIATYYCQQWSSNPFTFGQGTKLQITRTVAAPSVFIFPPSD
EQLKSGTASVVCLLNNFYPREAKVQWKVDNALQSGNSQESVTEQDSKDSTYSLCSTLT
LSKADYEKHKVYACEVTHQGLSSPVTKSFNRGES
Anti-CD8 TRX2 bDS Fab sequence
TRX2 bDS HC (SEQ ID NO: 56):
QVQLVESGGGVVQPGRSLRLSCAASGFTFSDFGMNWVRQAPGKGLEWVALIYYDGSN
KFYADSVKGRFTISRDNSKNTLYLQMNSLRAEDTAVYYCAKPHYDGYYHFFDSWGQG
TLVTVSSASTKGPSVFPLAPSSKSTSGGTAALGCLVKDYFPEPVTVSWNSGALTSGVHTC
PAVLQSSGLYSLSSVVTVPSSSLGTQTYICNVNHKPSNTKVDKKVEPKSSDKTHTC
TRX2 bDS LC (SEQ ID NO: 57):
DIQMTQSPSSLSASVGDRVTITCKGSQDINNYLAWYQQKPGKAPKLLIYNTDILHTGVPS
RFSGSGSGTDFTFTISSLQPEDIATYYCYQYNNGYTFGQGTKVEIKRTVAAPSVFIFPPSD
EQLKSGTASVVCLLNNFYPREAKVQWKVDNALQSGNSQESVTEQDSKDSTYSLCSTLT
LSKADYEKHKVYACEVTHQGLSSPVTKSFNRGES
Anti-CD2 Lo-CD2b bDS Fab sequence
Lo-CD2b bDS HC (SEQ ID NO: 58);
EVQLVESGGGLVQPGASLKLSCVASGFTFSDYWMSWVRQTPGKPMEWIGHIKYDGSYT
NYAPSLKNRFTISRDNAKTTLYLQMSNVRSEDSATYYCAREAPGAASYWGQGTLVTVS
SASTKGPSVFPLAPSSKSTSGGTAALGCLVKDYFPEPVTVSWNSGALTSGVHTCPAVLQS
SGLYSLSSVVTVPSSSLGTQTYICNVNHKPSNTKVDKKVEPKSSDKTHTC
Lo-CD2b bDS LC (SEQ ID NO: 59):
DVVLTQTPVAQPVTLGDQASISCRSSQSLVHSNGNTYLEWFLQKPGQSPQLLIYKVSNRF
SGVPDRFIGSGSGSDFTLKISRVEPEDWGVYYCFQGTHDPYTFGAGTKLELKRTVAAPS
VFIFPPSDEQLKSGTASVVCLLNNFYPREAKVQWKVDNALQSGNSQESVTEQDSKDSTY
SLCSTLTLSKADYEKHKVYACEVTHQGLSSPVTKSFNRGES
Anti-CD2 35.1 bDS Fab sequence
35.1 bDS HC (SEQ ID NO: 60):
EVQLQQSGAELVKPGASVKLSCRTSGFNIKDTYIHWVKQRPEQGLKWIGRIDPANGNTK
YDPKFQDKATVTADTSSNTAYLQLSSLTSEDTAVYYCVTYAYDGNWYFDVWGAGTAV
TVSSASTKGPSVFPLAPSSKSTSGGTAALGCLVKDYFPEPVTVSWNSGALTSGVHTCPAV
LQSSGLYSLSSVVTVPSSSLGTQTYICNVNHKPSNTKVDKKVEPKSSDKTHTC
35.1 bDS LC (SEQ ID NO: 61):
DIKMTQSPSSMYVSLGERVTITCKASQDINSFLSWFQQKPGKSPKTLIYRANRLVDGVPS
RFSGSGSGQDYSLTISSLEYEDMEIYYCLQYDEFPYTFGGGTKLEMKRTVAAPSVFIFPPS
DEQLKSGTASVVCLLNNFYPREAKVQWKVDNALQSGNSQESVTEQDSKDSTYSLCSTL
TLSKADYEKHKVYACEVTHQGLSSPVTKSFNRGES
Anti-CD2 OKT11 bDS Fab sequence
OKT11 bDS HC (SEQ ID NO: 62):
QVQLQQPGAELVRPGTSVKLSCKASGYTFTSYWMHWIKQRPEQGLEWIGRIDPYDSET
HYNEKFKDKAILSVDKSSSTAYIQLSSLTSDDSAVYYCSRRDAKYDGYALDYWGQGTS
VTVSSASTKGPSVFPLAPSSKSTSGGTAALGCLVKDYFPEPVTVSWNSGALTSGVHTCPA
VLQSSGLYSLSSVVTVPSSSLGTQTYICNVNHKPSNTKVDKKVEPKSSDKTHTC
OKT11 bDS LC (SEQ ID NO: 63):
DIVMTQAAPSVPVTPGESVSISCRSSKTLLHSNGNTYLYWFLQRPGQSPQVLIYRMSNLA
SGVPNRFSGSGSETTFTLRISRVEAEDVGIYYCMQHLEYPYTFGGGTKLEIERTVAAPSVF
IFPPSDEQLKSGTASVVCLLNNFYPREAKVQWKVDNALQSGNSQESVTEQDSKDSTYSL
CSTLTLSKADYEKHKVYACEVTHQGLSSPVTKSFNRGES
Anti-CD11a HzMHM24 bDS Fab sequence
HzMHM24 bDS HC (SEQ ID NO: 64):
EVQLVESGGGLVQPGGSLRLSCAASGYSFTGHWMNWVRQAPGKGLEWVGMIHPSDSE
TRYNQKFKDRFTISVDKSKNTLYLQMNSLRAEDTAVYYCARGIYFYGTTYFDYWGQGT
LVTVSSASTKGPSVFPLAPSSKSTSGGTAALGCLVKDYFPEPVTVSWNSGALTSGVHTCP
AVLQSSGLYSLSSVVTVPSSSLGTQTYICNVNHKPSNTKVDKKVEPKSSDKTHTCHHHH
HH
HzMHM24 bDS LC (SEQ ID NO: 65):
DIQMTQSPSSLSASVGDRVTITCRASKTISKYLAWYQQKPGKAPKLLIYSGSTLQSGVPS
RFSGSGSGTDFTLTISSLQPEDFATYYCQQHNEYPLTFGQGTKVEIKRTVAAPSVFIFPPS
DEQLKSGTASVVCLLNNFYPREAKVQWKVDNALQSGNSQESVTEQDSKDSTYSLCSTL
TLSKADYEKHKVYACEVTHQGLSSPVTKSFNRGES
Anti-CD18 h1B4 bDS Fab sequence
h1B4 bDS HC (SEQ ID NO: 66):
EVQLVESGGDLVQPGRSLRLSCAASGFTFSDYYMSWVRQAPGKGLEWVAAIDNDGGSI
SYPDTVKGRFTISRDNAKNSLYLQMNSLRVEDTALYYCARQGRLRRDYFDYWGQGTL
VTVSTASTKGPSVFPLAPSSKSTSGGTAALGCLVKDYFPEPVTVSWNSGALTSGVHTCP
AVLQSSGLYSLSSVVTVPSSSLGTQTYICNVNHKPSNTKVDKKVEPKSSDKTHTCHHHH
HH
h1B4 bDS LC (SEQ ID NO: 67):
DIQMTQSPSSLSASVGDRVTITCRASESVDSYGNSFMHWYQQKPGKAPKLLIYRASNLE
SGVPSRFSGSGSGTDFTFTISSLQPEDIATYYCQQSNEDPLTFGQGTKLEIKRTVAAPSVFI
FPPSDEQLKSGTASVVCLLNNFYPREAKVQWKVDNALQSGNSQESVTEQDSKDSTYSL
CSTLTLSKADYEKHKVYACEVTHQGLSSPVTKSFNRGES
Anti-CD18 Erlizumab bDS Fab sequence
Erlizumab bDS HC (SEQ ID NO: 68):
EVQLVESGGGLVQPGGSLRLSCATSGYTFTEYTMHWMRQAPGKGLEWVAGINPKNGG
TSHNQRFMDRFTISVDKSTSTAYMQMNSLRAEDTAVYYCARWRGLNYGFDVRYFDVW
GQGTLVTVSSASTKGPSVFPLAPSSKSTSGGTAALGCLVKDYFPEPVTVSWNSGALTSG
VHTCPAVLQSSGLYSLSSVVTVPSSSLGTQTYICNVNHKPSNTKVDKKVEPKSSDKTHTC
HHHHHH
Erlizumab bDS LC (SEQ ID NO: 69):
DIQMTQSPSSLSASVGDRVTITCRASQDINNYLNWYQQKPGKAPKLLIYYTSTLHSGVPS
RFSGSGSGTDYTLTISSLQPEDFATYYCQQGNTLPPTFGQGTKVEIKRTVAAPSVFIFPPS
DEQLKSGTASVVCLLNNFYPREAKVQWKVDNALQSGNSQESVTEQDSKDSTYSLCSTL
TLSKADYEKHKVYACEVTHQGLSSPVTKSFNRGES
Anti-CD4/CD8 Ibalizumab/TRX2 bDS Fab-ScFv sequence
Ibalizumab/TRX2 bDS Fab-ScFv HC (SEQ ID NO: 70):
QVQLQQSGPEVVKPGASVKMSCKASGYTFTSYVIHWVRQKPGQGLDWIGYINPYNDGT
DYDEKFKGKATLTSDTSTSTAYMELSSLRSEDTAVYYCAREKDNYATGAWFAYWGQG
TLVTVSSASTKGPSVFPLAPSSKSTSGGTAALGCLVKDYFPEPVTVSWNSGALTSGVHTC
PAVLQSSGLYSLSSVVTVPSSSLGTQTYICNVNHKPSNTKVDKKVEPKSSDKTHTCHHH
HHH
Ibalizumab/TRX2 bDS Fab-ScFv LC (SEQ ID NO: 71):
DIVMTQSPDSLAVSLGERVTMNCKSSQSLLYSTNQKNYLAWYQQKPGQSPKLLIYWAS
TRESGVPDRFSGSGSGTDFTLTISSVQAEDVAVYYCQQYYSYRTFGGGTKLEIKRTVAAP
SVFIFPPSDEQLKSGTASVVCLLNNFYPREAKVQWKVDNALQSGNSQESVTEQDSKDST
YSLCSTLTLSKADYEKHKVYACEVTHQGLSSPVTKSFNRGESGGGGSGGGGSGGGGSQ
VQLVESGGGVVQPGRSLRLSCAASGFTFSDFGMNWVRQAPGKGLEWVALIYYDGSNK
FYADSVKGRFTISRDNSKNTLYLQMNSLRAEDTAVYYCAKPHYDGYYHFFDSWGQGT
LVTVSSGGGGSGGGGSGGGGSGGGGSDIQMTQSPSSLSASVGDRVTITCKGSQDINNYL
AWYQQKPGKAPKLLIYNTDILHTGVPSRFSGSGSGTDFTFTISSLQPEDIATYYCYQYNN
GYTFGQGTKVEIK
Anti-CD4 Ibalizumab NoDS Fab sequence
Ibalizumab NoDS LC (SEQ ID NO: 72):
QVQLQQSGPEVVKPGASVKMSCKASGYTFTSYVIHWVRQKPGQGLDWIGYINPYNDGT
DYDEKFKGKATLTSDTSTSTAYMELSSLRSEDTAVYYCAREKDNYATGAWFAYWGQG
TLVTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSSDKTHTC
Ibalizumab NoDS HC (SEQ ID NO: 73):
DIVMTQSPDSLAVSLGERVTMNCKSSQSLLYSTNQKNYLAWYQQKPGQSPKLLIYWAS
TRESGVPDRFSGSGSGTDFTLTISSVQAEDVAVYYCQQYYSYRTFGGGTKLEIKRTVAAP
SVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGES
Anti-CD4 OKT4 bDS Fab sequence
OKT4 bDS LC (SEQ ID NO: 74):
EVQLVESGGGLVQPGGSLRLSCAASGFTFSNYAMSWVRQAPGKRLEWVSAISDHSTNT
YYPDSVKGRFTISRDNAKNTLYLQMNSLRAEDTAVYYCARKYGGDYDPFDYWGQGTL
VTVSSASTKGPSVFPLAPSSKSTSGGTAALGCLVKDYFPEPVTVSWNSGALTSGVHTCPA
VLQSSGLYSLSSVVTVPSSSLGTQTYICNVNHKPSNTKVDKKVEPKSSDKTHTCHHHHH
H
OKT4 bDS HC (SEQ ID NO: 75):
DIQMTQSPSSLSASVGDRVTITCQASQDINNYIAWYQHKPGKGPKLLIHYTSTLQPGIPSR
FSGSGSGRDYTLTISSLQPEDFATYYCLQYDNLLFTFGGGTKVEIKRTVAAPSVFIFPPSD
EQLKSGTASVVCLLNNFYPREAKVQWKVDNALQSGNSQESVTEQDSKDSTYSLCSTLT
LSKADYEKHKVYACEVTHQGLSSPVTKSFNRGES
Anti-CD4 T023200008 Nb sequence (SEQ ID NO: 76)
CDR1, CDR2, CDR3 underlined based on IMGT designation:
EVQLVESGGGSVQPGGSLTLSCGTSGRTFNVMGWFRQAPGKEREFVAAVRWSSTGIYY
TQYADSVKSRFTISRDNAKNTVYLEMNSLKPEDTAVYYCAADTYNSNPARWDGYDER
GQGTLVTVSSGGCGGHHHHHH
Anti-CD8 BDSn Nb sequence (SEQ ID NO: 77)
CDR1, CDR2, CDR3 underlined based on IMGT designation:
EVQLVESGGGLVQAGGSLRLSCAASGSTFSDYGVGWFRQAPGKGREFVADIDWNGEHT
SYADSVKGRFATSRDNAKNTAYLQMNSLKPEDTAVYYCAADALPYTVRKYNYWGQGT
QVTVSSGGCGGHHHHHH
Anti-CD3 T0170117G03-A Nb sequence (SEQ ID NO: 78)
EVQLVESGGGPVQAGGSLRLSCAASGRTYRGYSMGWFRQAPGKEREFVAAIVWSGGN
TYYEDSVKGRFTISRDNAKNIMYLQMTSLKPEDSATYYCAAKIRPYIFKIAGQYDYWGQ
GTLVTVSSAGGGSGGHHHHHHC
Anti-CD3 T0170060E11 Nb sequence (SEQ ID NO: 79)
EVQLVESGGGLVQPGGSLRLSCAASGDIYKSFDMGWYRQAPGKQRDLVAVIGSRGNNR
GRTNYADSVKGRFTISRDGTGNTVYLLMNKLRPEDTAIYYCNTAPLVAGRPWGRGTLV
TVSSGGGSGGHHHHHHC
Anti-CD7 V1 Nb sequence (SEQ ID NO: 80)
DVQLQESGGGLVQAGGSLRLSCAVSGYPYSSYCMGWFRQAPGKEREGVAAIDSDGRTR
YADSVKGRFTISQDNAKNTLYLQMNRMKPEDTAMYYCAARFGPMGCVDLSTLSFGHW
GQGTQVTVSITGGGCHHHHHHHH
Anti-TCR T017000700 Nb sequence (SEQ ID NO: 81)
CDR1, CDR2, CDR3 underlined based on IMGT designation:
EVQLVESGGGVVQPGGSLRLSCVASGYVHKINFYGWYRQAPGKEREKVAHISIGDQTD
YADSAKGRFTISRDESKNTVYLQMNSLRPEDTAAYYCRALSRIWPYDYWGQGTLVTVS
SGGCGGHHHHHH
Anti-CD28 28CD065G01 Nb sequence (SEQ ID NO: 82)
EVQLVESGGGLVQPGGSLRLSCAASGSIFRLHTMEWYRRTPETQREWVATITSGGTTNY
PDSVKGRFTISRDDTKKTVYLQMNSLKPEDTAVYYCHAVATEDAGFPPSNYWGQGTLV
TVSSGGCGGHHHHHH
Anti-CD3 T0170061C09 Nb sequence (SEQ ID NO: 83)
EVQLVESGGGPVQAGGSLRLSCAASGRTYRGYSMGWFRQAPGREREFVAAIVWSDGN
TYYEDSVKGRFTISRDNAKNTMYLQMTSLKPEDSATYYCAAKIRPYIFKIAGQYDYWG
QGTLVTVSSGGCGGHHHHHH
Anti-CD3 12D2 bDS Fab sequence
12D2 bDS HC (SEQ ID NO: 84):
EVKLVESGGGLVQPGRSLRLSCAASGFNFYAYWMGWVRQAPGKGLEWIGEIKKDGTTI
NYTPSLKDRFTISRDNAQNTLYLQMTKLGSEDTALYYCAREERDGYFDYWGQGVMVT
VSSASTKGPSVFPLAPSSKSTSGGTAALGCLVKDYFPEPVTVSWNSGALTSGVHTCPAVL
QSSGLYSLSSVVTVPSSSLGTQTYICNVNHKPSNTKVDKKVEPKSSDKTHTCGGHHHHH
H
12D2 bDS LC (SEQ ID NO: 85):
QFVLTQPNSVSTNLGSTVKLSCKRSTGNIGSNYVNWYQQHEGRSPTTMIYRDDKRPDG
VPDRFSGSIDRSSNSALLTINNVQTEDEADYFCQSYSSGIVFGGGTKLTVLSQPKAAPSVT
LFPPSSEELQANKATLVCLVSDFYPGAVTVAWKADGSPVKVGVETTKPSKQSNNKYAA
CSYLSLTPEQWKSHRSYSCRVTHEGSTVEKTVAPAESS
Anti-CD28 8G8A Fab sequence
8G8A bDS HC (SEQ ID NO: 86):
EVQLQQSGPELVKPGASVKMSCKASGYTFTSYVIQWVKQKPGQGLEWIGSINPYNDYT
KYNEKFKGKATLTSDKSSITAYMEFSLTSEDSALYCARWGDGNYWGRGTLTVSSASTK
GPSVFPLAPSSKSTSGGTAALGCLVKDYFPEPVTVSWNSGALTSGVHTCPAVLQSSGLYS
LSSVVTVPSSSLGTQTYICNVNHKPSNTKVDKKVEPKSSDKTHTCGGHHHHHH
8G8A bDS LC (SEQ ID NO: 87):
DIEMTQSPAIMSASLGERVTMTCTASSSVSSSYFHWYQKPGSSPKLCIYSTSNLASGVPPR
FSGSGSTSYSLTISMEAEDAATYFCHQYHRSPTFGGGTKLETKRTVAAPSVFIFPPSDEQL
KSGTASVVCLLNNFYPREAKVQWKVDNALQSGNSQESVTEQDSKDSTYSLCSTLTLSK
ADYEKHKVYACEVTHQGLSSPVTKSFNRGES
Anti-CD28 2E12 Fab sequence
2E12 bDS HC (SEQ ID NO: 88):
QVQLKESGPGLVAPSQSLSITCTVSGFSLTGYGVNWVRQPPGKGLEWLGMIWGDGSTD
YNSALKSRLSITKDNSKSQVFLKMNSLQTDDTARYYCARDGYSNFHYYVMDYWGQGT
SVTVSSASTKGPSVFPLAPSSKSTSGGTAALGCLVKDYFPEPVTVSWNSGALTSGVHTCP
AVLQSSGLYSLSSVVTVPSSSLGTQTYICNVNHKPSNTKVDKKVEPKSSDKTHTCGGHH
HHHH
2E12 bDS LC (SEQ ID NO: 89):
DIVLTQSPASLAVSLGQRATISCRASESVEYYVTSLMQWYQQKPGQPPKLLISAASNVES
GVPARFSGSGSGTDFSLNIHPVEEDDIAMYFCQQSRKVPWTFGGGTKLEIKRRTVAAPSV
FIFPPSDEQLKSGTASVVCLLNNFYPREAKVQWKVDNALQSGNSQESVTEQDSKDSTYS
LCSTLTLSKADYEKHKVYACEVTHQGLSSPVTKSFNRGES
Anti-CD28 CD28.9.3 Fab sequence
CD28.9.3 bDS HC (SEQ ID NO: 90):
QVKLQQSGPGLVTPSQSLSITCTVSGFSLSDYGVHWVRQSPGQGLEWLGVIWAGGGTN
YNSALMSRKSISKDNSKSQVELKMNSLQADDTAVYYCARDKGYSYYYSMDYWGQGT
TVTVSSASTKGPSVFPLAPSSKSTSGGTAALGCLVKDYFPEPVTVSWNSGALTSGVHTCP
AVLQSSGLYSLSSVVTVPSSSLGTQTYICNVNHKPSNTKVDKKVEPKSSDKTHTCGGHH
HHHH
CD28.9.3 bDS LC (SEQ ID NO: 91):
DIVLTQSPAS LAVSLGQRAT ISCRASESVEYYVTSLMQWY QQKPGQPPKL
LIFAASNVES GVPARFSGSG SGTNFSLNIHPVDEDDVAMY FCQQSRKVPY
TFGGGTKLEIKRTVAAPSVFIFPPSDEQLKSGTASVVCLLNNFYPREAKVQWKVDNALQ
SGNSQESVTEQDSKDSTYSLCSTLTLSKADYEKHKVYACEVTHQGLSSPVTKSFNRGES
Anti-CD28 HzTN228 Fab sequence
HzTN228 bDS HC (SEQ ID NO: 92):
QVQLQESGPGLVKPSETLSLTCAVSGFSLTSYGVHWIRQPGKGLEWLGVIWPGTNFNSA
LMSRLTISEDTSKNQVSLKLSSVTAADTAVYCARDRAYGNYLYAMDYWGQGTLVTVS
SASTKGPSVFPLAPSSKSTSGGTAALGCLVKDYFPEPVTVSWNSGALTSGVHTCPAVLQS
SGLYSLSSVVTVPSSSLGTQTYICNVNHKPSNTKVDKKVEPKSSDKTHTCGGHHHHHH
HzTN228 bDS LC (SEQ ID NO: 93):
DIQMTQSPSLSASVGDRVTITCRASESVEYVTSLMQWYQKPGKAPKLLIYAASNVDSGV
PSRFSGSGTDFTLTISLQPEDIATYCQSRKVPFTFGGGTKVEIKRTVAAPSVFIFPPSDEQL
KSGTASVVCLLNNFYPREAKVQWKVDNALQSGNSQESVTEQDSKDSTYSLCSTLTLSK
ADYEKHKVYACEVTHQGLSSPVTKSFNRGES
Anti-CD28 TGN2122.C Fab sequence
TGN2122.C bDS HC (SEQ ID NO: 94):
QVQLVQSGAEVKKPGASVKVSCKASGYTFTDYKIHWVRQAPGQGLEWIGYIYPYSGSS
DYNQKFKSRATLTVDNSISTAYMELSRLRSDDTAVYYCARGGDAMDYWGQGTLVTVS
SASTKGPSVFPLAPSSKSTSGGTAALGCLVKDYFPEPVTVSWNSGALTSGVHTCPAVLQS
SGLYSLSSVVTVPSSSLGTQTYICNVNHKPSNTKVDKKVEPKSSDKTHTCGGHHHHHH
TGN2122.C bDS LC (SEQ ID NO: 95);
DIQMTQSPSSLSASVGDRVTITCGASENIYGALNWYQRKPGKAPKLLIYGATNLADGVP
SRFSGSGSGRDYTLTISSLQPEDFATYFCQNILGTWTFGGGTKVEIKRTVAAPSVFIFPPSD
EQLKSGTASVVCLLNNFYPREAKVQWKVDNALQSGNSQESVTEQDSKDSTYSLCSTLT
LSKADYEKHKVYACEVTHQGLSSPVTKSFNRGES
Anti-CD28 TGN2122.H Fab sequence
TGN2122.H bDS HC (SEQ ID NO: 96):
EVQLVESGGGLVQPGGSLRLSCAASGFTFNIYYMSWVRQAPGKGLELVAAINPDGGNT
YYPDTVKGRFTISRDNAKNSLYLQMNSLRAEDTAVYYCARYGGPGFDSWGQGTLVTV
SSASTKGPSVFPLAPSSKSTSGGTAALGCLVKDYFPEPVTVSWNSGALTSGVHTCPAVLQ
SSGLYSLSSVVTVPSSSLGTQTYICNVNHKPSNTKVDKKVEPKSSDKTHTCGGHHHHHH
TGN2122.H bDS LC (SEQ ID NO: 97):
ENVLTQSPATLSLSPGERATLSCSASSSVSYMHWYQQKPGQAPRLWIYDTSKLASGIPAR
FSGSGSRNDYTLTISSLEPEDFAVYYCFPGSGFPFMYTFGGGTKVEIKRTVAAPSVFIFPPS
DEQLKSGTASVVCLLNNFYPREAKVQWKVDNALQSGNSQESVTEQDSKDSTYSLCSTL
TLSKADYEKHKVYACEVTHQGLSSPVTKSFNRGES
Anti-CD8 TRX2 ScFv sequence (SEQ ID NO: 98):
QVQLVESGGGVVQPGRSLRLSCAASGFTFSDFGMNWVRQAPGKGLEWVALIYYDGSN
KFYADSVKGRFTISRDNSKNTLYLQMNSLRAEDTAVYYCAKPHYDGYYHFFDSWGQG
TLVTVSSGGGGSGGGGSGGGGSGGGGSDIQMTQSPSSLSASVGDRVTITCKGSQDINNY
LAWYQQKPGKAPKLLIYNTDILHTGVPSRFSGSGSGTDFTFTISSLQPEDIATYYCYQYN
NGYTFGQGTKVEIKGGGSGGCGGHHHHHH
V1 VHH-CH1 bDS HC (SEQ ID NO: 99):
DVQLQESGGGLVQAGGSLRLSCAVSGYPYSSYCMGWFRQAPGKEREGVAAIDSDGRTR
YADSVKGRFTISQDNAKNTLYLQMNRMKPEDTAMYYCAARFGPMGCVDLSTLSFGHW
GQGTQVTVSITASTKGPSVFPLAPSSKSTSGGTAALGCLVKDYFPEPVTVSWNSGALTSG
VHTCPAVLQSSGLYSLSSVVTVPSSSLGTQTYICNVNHKPSNTKVDKKVEPKSSDKTHTC
GGHHHHHH

In some embodiments, the targeting moiety comprises a polypeptide sequence as disclosed herein. In some embodiments, the targeting moiety comprises all six CDRs of a polypeptide sequence as disclosed herein. In some embodiments, the targeting moiety comprises CDR₁, CDR₂, and CDR₃ of an immunoglobulin single variable domain (ISVD) as disclosed herein. In further embodiments, the targeting moiety binds to the same epitope on the targeting molecule that a polypeptide sequence as disclosed herein binds to. In further embodiments, the targeting moiety competes with a polypeptide sequence as disclosed herein to bind to the same epitope on the targeting molecule.

In certain embodiments, the targeting group or cell targeting group (e.g., a hematopoietic stem cell targeting agent or an immune cell targeting agents such as a T cell-targeting agent, B cell-targeting agent, NK-cell targeting agent, or macrophage-targeting agent) may be covalently coupled to a lipid via a polyethylene glycol (PEG) containing linker.

In other embodiments, the lipid used to create a conjugate may be selected from distearoyl-phosphatidylethanolamine (DSPE):

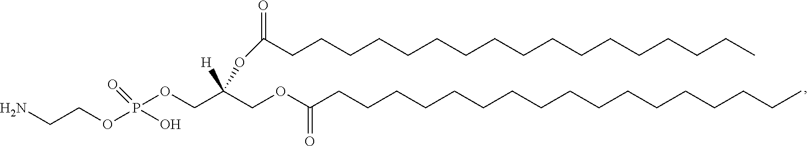 [SMILES: CCCCCCCCCCCCCCCCCC(=O)OC[C@H](COP(=O)(O)OCCN)OC(=O)CCCCCCCCCCCCCCCCC]

dipalmitoyl-phosphatidylethanolamine (DPPE):

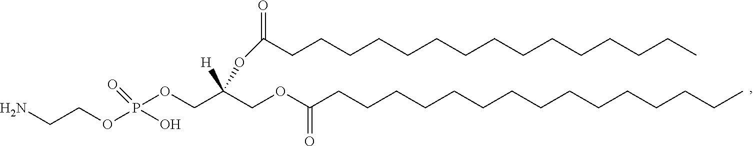 [SMILES: CCCCCCCCCCCCCCCC(=O)OC[C@H](COP(=O)(O)OCCN)OC(=O)CCCCCCCCCCCCCCC]

dimyrstoyl-phosphatidylethanolamine (DMPE):

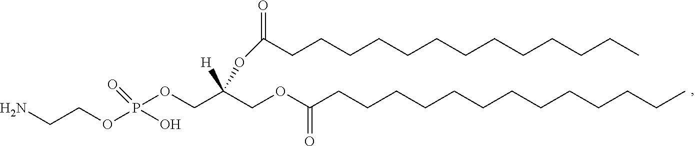 [SMILES: CCCCCCCCCCCCCC(=O)OC[C@H](COP(=O)(O)OCCN)OC(=O)CCCCCCCCCCCCC]

distearoyl-glycero-phosphoglycerol (DSPG):

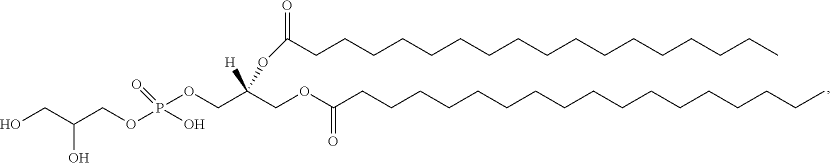 [SMILES: CCCCCCCCCCCCCCCCCC(=O)OC[C@H](COP(=O)(O)OCC(O)CO)OC(=O)CCCCCCCCCCCCCCCCC]

dimyristoyl-glycerol (DMG):

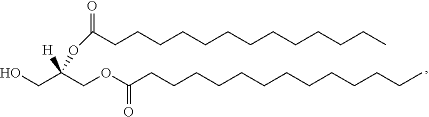 [SMILES: CCCCCCCCCCCCCC(=O)OC[C@H](CO)OC(=O)CCCCCCCCCCCCC]

distearoylglycerol (DSG):

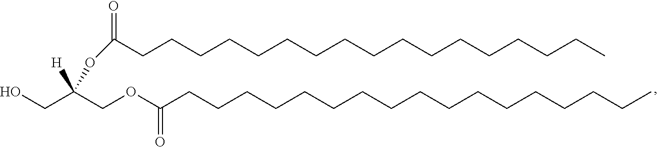 [SMILES: CCCCCCCCCCCCCCCCCC(=O)OC[C@H](CO)OC(=O)CCCCCCCCCCCCCCCCC]

and N-palmitoyl-sphingosine (C16-ceramide)

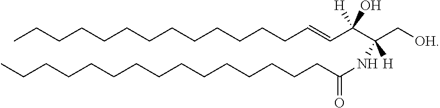 [SMILES: CCCCCCCCCCCCC/C=C/[C@@H](O)[C@H](CO)NC(=O)CCCCCCCCCCCCCCC]

The cell targeting group can be covalently coupled to a lipid either directly or via a linker, for example, a polyethylene glycol (PEG) containing linker. In certain embodiments, the PEG is PEG 1000, PEG 2000, PEG 3400, PEG 3000, PEG 3450, PEG 4000, or PEG 5000. In certain, embodiments, the PEG is PEG 2000.

In some embodiments, the lipid-cell targeting group conjugate is present in the lipid blend in a range of 0.001-0.5 mole percent, 0.001-0.3 mole percent, 0.002-0.2 mole percent, 0.01-0.1 mole percent, 0.1-0.3 mole percent, or 0.1-0.2 mole percent.

In certain embodiments, the lipid-cell targeting agent conjugate comprises DSPE, a PEG component and a targeting antibody. In certain embodiments, the antibody is an immune cell targeting agent, such as a T-cell targeting agent, for example, an anti-CD2 antibody, an anti-CD3 antibody, an anti-CD4 antibody, an anti-CDS antibody, an anti-CD7 antibody, an anti CD8 antibody, or an anti-TCR β antibody. In certain embodiments, the antibody is an hematopoietic stem cell targeting agent, such as, an anti-CD34 antibody, an anti-CD105 antibody, or an anti-CD117 antibody.

An exemplary lipid-cell targeting group conjugate comprises DSPE and PEG 2000, for example, as described in Nellis et al. (2005) BIOTECHNOL. PROG. 21, 205-220. An exemplary conjugate comprises the structure of Formula (III), where the scFv represents an engineered antibody binding site that binds to a target of interest. In certain embodiments, the engineered antibody binding site binds to any of the targets described hereinabove. In certain embodiments, the engineered antibody binding site can be, for example, an engineered anti-CD3 antibody or an engineered anti-CD8 antibody. In certain embodiments, the engineered antibody binding site can be, for example, an engineered anti-CD2 antibody or an engineered anti-CD7 antibody.

An example of a compound of Formula (III) is as shown below:

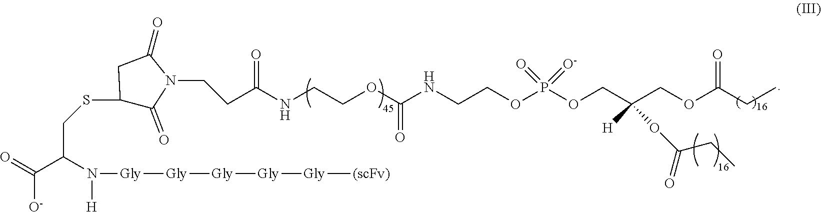

It is contemplated that the scFv in Formula (III) may be replaced with an intact antibody or an antigen fragment thereof (e.g., a Fab).

Another example of a compound of Formula (IV) is as shown below:

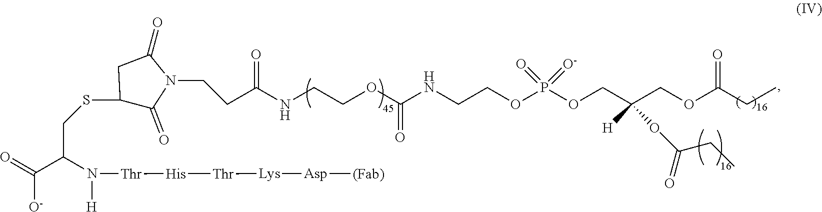

the production of which is described in Nellis et al. (2005) supra, or U.S. Pat. No. 7,022,336. It is contemplated that the Fab in Formula (IV) may be replaced with an intact antibody or an antigen fragment thereof (e.g., an (Fab′)₂ fragment) or an engineering antibody binding site (e.g., an scFv).

Other lipid immune cell target group conjugates are described, for example, in U.S. Pat. No. 7,022,336, where the targeting group may be replaced with a targeting group of interest, for example, a targeting group that binds a T-cell or NK cell surface antigen as described hereinabove.

In certain embodiments, the lipid component of an exemplary conjugate of Formula (II) can be any of the lipids described herein. In some embodiments, the lipid component of a conjugate of Formula (II) is based on an ionizable, cationic lipid described herein, for example, an ionizable, cationic lipid of Formula (I), Formula (I-P1), Formula (I-P2), or a slat thereof. For example, an exemplary ionizable, cationic lipid can be selected from Table 1, or a salt thereof.

In certain embodiments, the conjugate based on a lipid of the present disclosure may include:

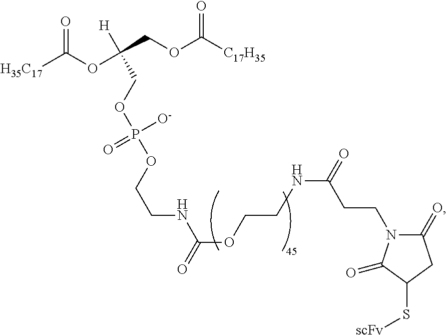

where scFv represents an engineered antibody binding site that binds a target described hereinabove, e.g., CD2, CD3, CD7, or CD8.

In certain embodiments, the lipid blend may further comprise free PEG-lipid so as to reduce the amount of non-specific binding via the targeting group. The free PEG-lipid can be the same or different from the PEG-lipid included in the conjugate. In certain embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl [methoxy (polyethylene glycol)] (PEG-ceramide), DSPE-PEG-cysteine, or a derivative thereof, all with average PEG lengths between 2000-5000, with 2000, 3400, or 5000. A final composition may comprise a mixture of two or more of these pegylated lipids. In certain embodiments, the LNP composition comprises a mixture of PEG-lipids with myristoyl and stearic acyl chains. In certain embodiments, the LNP composition comprises a mixture of PEG-lipids with palmitoyl and stearoyl acyl chains.

In certain embodiments, the derivative of the PEG-lipid has a methyoxy, hydroxyl or a carboxylic acid end group at the PEG terminus.

The lipid-cell targeting group conjugate can be incorporated into LNPs as described below, for example, in LNPs comprising, for example, an ionizable cationic lipid, a sterol, a neutral phospholipid and a PEG-lipid. It is contemplated that, in certain embodiments, the LNPs comprising the lipid-cell targeting group can comprise an ionizable cationic lipid described herein or a cationic lipid described, for example, in U.S. Pat. Nos. 10,221,127, 10,653,780 or U.S. Published application No. US2018/0085474, US2016/0317676, International Publication No. WO2009/086558, or Miao et al. (2019) NATURE BIOTECH 37:1174-1185, or Jayaraman et al. (2012) ANGEW CHEM INT. 51:8529-8533.

The LNPs can be formulated using the methods and other components described below in the following sections.

### IV. Lipid Nanoparticle Compositions

The invention provides a lipid nanoparticle (LNP) composition comprising a lipid blend that comprises an ionizable cationic lipid described herein and/or a lipid-cell targeting agent (e.g., a lipid-immune cell targeting agent) conjugate described herein. In certain embodiments, the lipid blend may comprise an ionizable, cationic lipid described herein and one or more of a sterol, a neutral phospholipid, a PEG-lipid, and a lipid-cell targeting group conjugate.

In certain embodiments, the ionizable, cationic lipid described herein may be present in the lipid blend in a range of 30-70 mole percent, 30-60 mole percent 30-50 mole percent, 40-70 mole percent, 40-60 mole percent, 40-50 mole percent, 50-70 mole percent, 50-60 mole percent, or of about 30 mole percent, about 35 mole percent, about 40 mole percent, about 45 mole percent, about 50 mole percent, about 55 mole percent, about 60 mole percent, about 65 mole percent, or about 70 mole percent.

### Sterol

In certain embodiments, the lipid blend of the lipid nanoparticle may comprise a sterol component, for example, one or more sterols selected from the group consisting of cholesterol, fecosterol, β-sitosterol, ergosterol, campesterol, stigmasterol, stigmastanol, brassicasterol. In certain embodiments, the sterol is cholesterol.

The sterol (e.g., cholesterol) may be present in the lipid blend in a range of 20-70 mole percent, 20-60 mole percent, 20-50 mole percent, 30-70 mole percent, 30-60 mole percent, 30-50 mole percent, 40-70 mole percent, 40-60 mole percent, 40-50 mole percent, 50-70 mole percent, 50-60 mole percent, or about 20 mole percent, about 25 mole percent, about 30 mole percent, about 35 mole percent, about 40 mole percent, about 45 mole percent, about 50 mole percent, about 55 mole percent, about 60 mole percent or about 65 mole percent.

### Neutral Phospholipid

In certain embodiments, the lipid blend of the lipid nanoparticle may comprise one or more neutral phospholipids. The neutral phospholipid can be selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), 1,2-dioleoyl-sn-glycero-3-phosphocholine (DOPC), sphingomyelin (SM).

Other neutral phospholipids can be selected from the group consisting of distearoyl-phosphatidylethanolamine (DSPE), dimyrstoyl-phosphatidylethanolamine (DMPE), distearoyl-glycero-phosphocholine (DSPC), hydrogenated soy phosphatidylcholine (HSPC), dioleoyl-glycero-phosphoethanolamine (DOPE), dilinoleoyl-glycero-phosphocholine (DLPC), dimyristoyl-glycero-phosphocholine (DMPC), dioleoyl-glycero-phosphocholine (DOPC), dipalmitoyl-glycero-phosphocholine (DPPC), diundecanoyl-glycero-phosphocholine (DUPC), palmitoyl-oleoyl-glycero-phosphocholine (POPC), dioctadecenyl-glycero-phosphocholine, oleoyl-cholesterylhemisuccinoyl-glycero-phosphocholine, hexadecyl-glycero-phosphocholine, dilinolenoyl-glycero-phosphocholine, diarachidonoyl-glycero-3-phosphocholine, didocosahexaenoyl-glycero-phosphocholine, or sphingomyelin.

The neutral phospholipid may be present in the lipid blend in a range of 1-10 mole percent, 1-15 mole percent, 1-12 mole percent, 1-10 mole percent, 3-15 mole percent, 3-12 mole percent, 3-10 mole percent, 4-15 mole percent, 4-12 mole percent, 4-10 mole percent, 4-8 mole percent, 5-15 mole percent, 5-12 mole percent, 5-10 mole percent, 6-15 mole percent, 6-12 mole percent, 6-10 more percent, or about 1 mole percent, about 2 mole percent, about 3 mole percent, about 4 mole percent, about 5 mole percent, about 6 mole percent, about 7 mole percent, about 8 mole percent, about 9 mole percent, about 10 mole percent, about 11 mole percent, about 12 mole percent, about 13 mole percent, about 14 mole percent, or about 15 mole percent.

### PEG-Lipid

The lipid blend of the lipid nanoparticle may include one or more PEG or PEG-modified lipids. Such species may be alternately referred to as PEGylated lipids. A PEG lipid is a lipid modified with polyethylene glycol. As noted above, free PEG-lipids can be included in the lipid blend to reduce or eliminate non-specific binding via a targeting group when a lipid-cell targeting group is included in the lipid blend.

A PEG lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

In certain embodiments, the blend may comprise a free PEG-lipid that can be selected from the group consisting of PEG-distearoylglycerol (PEG-DSG), PEG-diacylglycerol (PEG-DAG, e.g., PEG-DMG, PEG-DPG, and PEG-DSG), PEG-dimyristoyl-glycerol (PEG-DMG), PEG-distearoyl-phosphatidylethanolamine (PEG-DSPE) and PEG-dimyrstoyl-phosphatidylethanolamine (PEG-DMPE). In some embodiments, the free PEG-lipid comprises a diacylphosphatidylcholines comprising Dipalmitoyl (C16) chain or Distearoyl (C18) chain.

The PEG-lipid may be present in the lipid blend in a range of 1-10 mole percent, 1-8 mole percent, 1-7 mole percent, 1-6 mole percent, 1-5 mole percent, 1-4 mole percent, 1-3 mole percent, 2-8 mole percent, 2-7 mole percent, 2-6 mole percent, 2-5 mole percent, 2-4 mole percent, 2-3 mole percent, or about 1 mole percent, about 2 mole percent, about 3 mole percent, about 4 mole percent, or about 5 mole percent. In some embodiments, the PEG-lipid is a free PEG-lipid.

In some embodiments, the PEG-lipid may be present in the lipid blend in the range of 0.01-10 mole percent, 0.01-5 mole percent, 0.01-4 mole percent, 0.01-3 mole percent, 0.01-2 mole percent, 0.01-1 mole percent, 0.1-10 mole percent, 0.1-5 mole percent, 0.1-4 mole percent, 0.1-3 mole percent, 0.1-2 mole percent, 0.1-1 mole percent, 0.5-10 mole percent, 0.5-5 mole percent, 0.5-4 mole percent, 0.5-3 mole percent, 0.5-2 mole percent, 0.5-1 mole percent, 1-2 mole percent, 3-4 mole percent, 4-5 mole percent, 5-6 mole percent, or 1.25-1.75 mole percent. In some embodiments, the PET-lipid may be about 0.5 mole percent, about 1 mole percent, about 1.5 mole percent, about 2 mole percent, about 2.5 mole percent, about 3 mole percent, about 3.5 mole percent, about 4 mole percent, about 4.5 mole percent, about 5 mole percent, or about 5.5 mole percent of the lipid blend. In some embodiments, the PEG-lipid is a free PEG-lipid.

In some embodiments, the lipid anchor length of PEG-lipid is C14 (as in PEG-DMG). In some embodiments, the lipid anchor length of PEG-lipid is C16 (as in DPG). In some embodiments, the lipid anchor length of PEG-lipid is C18 (as in PEG-DSG). In some embodiments, the back bone or head group of PEG-lipid is diacyl glycerol or phosphoethanolamine. In some embodiments, the PEG-lipid is a free PEG-lipid.

A LNP of the present disclosure may comprise one or more free PEG-lipid that is not conjugated to an cell targeting group, and a PEG-lipid that is conjugated to cell targeting group. In some embodiments, the free PEG-lipid comprises the same or a different lipid as the lipid in the lipid-cell targeting group conjugate.

### Cell Targeting Group Conjugate

In certain embodiments, the lipid blend can also include a lipid-cell targeting group conjugate.

The lipid-cell targeting group conjugate may be present in the lipid blend in a range of 0.001-0.5 mol percent, 0.001-0.1 mole percent, 0.01-0.5 mole percent, 0.05-0.5 mole percent, 0.1-0.5 mole percent, 0.1-0.3 mole percent, 0.1-0.2 mole percent, 0.2-0.3 mole percent, of about 0.01 mole percent, about 0.05 mole percent, about 0.1 mole percent, about 0.15 mole percent, about 0.2 mole percent, about 0.25 mole percent, about 0.3 mole percent, about 0.35 mole percent, about 0.4 mole percent, about 0.45 mole percent, or about 0.5 mole percent.

In addition to the lipids present in the lipid blend, the LNP compositions may further comprise a payload, for example, a payload described hereinbelow. In certain embodiments, the payload is a nucleic acid, for example, DNA or RNA, for example, an mRNA, transfer RNA (IRNA), a microRNA, or small interfering RNA (siRNA).

In certain embodiments, the number of the nucleotides in the nucleic acid is from about 400 to about 6000.

### Production of Lipid Nanoparticles

In some embodiments, the LNPs are produced by using either rapid mixing by an orbital vortexer or by microfluidic mixing. Orbital vortexer mixing is accomplished by rapid addition of lipids solution in ethanol to the aqueous solution of a nucleic acid of interest followed immediately by vortexing at 2,500 rpm. In some embodiments, the LNPs are produced using a microfluidic mixing step. In some embodiments, microfluidic mixing is achieved mixing the aqueous and organic streams at a controlled flow rates in a microfluidic channel using, e.g., a NanoAssemblr device and microfluidic chips featuring optimized mixing chamber geometry (Precision Nanosystems, Vancouver, BC). In some embodiments, the LNPs are produced using a microfluidic mixing step to rapidly mix the ethanolic lipid solution and aqueous nucleic acid solution, resulting in encapsulation of the nucleic acid in the solid lipid nanoparticles. The nanoparticle suspension is then buffer exchanged into an all aqueous buffer using membrane filtration device of choice for ethanol removal and nanoparticle maturation.

In certain embodiments, the resulting LNP compositions comprise a lipid blend comprising, for example, from about 40 mole percent to about 60 mole percent of one or more ionizable cationic lipids described herein, from about 35 mole percent to about 50 mole percent of one or more sterols, from about 5 mole percent to about 15 mole percent of one or more neutral lipids, and from about 0.5 mole percent to about 5 mole percent of one or more PEG-lipids.

### Physical Properties of Lipid Nanoparticles

The characteristics of an LNP composition may depend on the components, their absolute or relative amounts, contained in a lipid nanoparticle (LNP) composition. Characteristics may also vary depending on the method and conditions of preparation of the LNP composition.

LNP compositions may be characterized by a variety of methods. For example, microscopy (e.g., transmission electron microscopy or scanning electron microscopy) may be used to examine the morphology and size distribution of an LNP composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of an LNP composition, such as particle size, polydispersity index, and zeta potential. RNA encapsulated efficiency is determined by a combination of methods relying on RNA binding dyes (ribogreen, cybergreen to determine dye accessible RNA fraction) and LNP de-formulation followed by HPLC analysis for total RNA content.

In some embodiments, the LNP may have a mean diameter in the range of 1-250 nm, 1-200 nm, 1-150 nm, 1-100 nm, 50-250 nm, 50-200 nm, 50-150 nm, 50-100 nm, 75-250 nm, 75-200 nm, 75-150 nm, 75-100 nm, 100-250 nm, 100-200 nm, 100-150 nm. In certain embodiments, the LNP compositions may have a mean diameter of about Inm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the LNP has a mean diameter of about 100 nm.

In some embodiments, LNPs comprising an ionizable cationic lipid described herein, prepared and characterized using methods described herein, show average diameter change after a freeze-thaw of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change after a freeze-thaw of less than 30%. In some embodiments, the freeze-thaw and diameter measurements are conducted with 10% sucrose in MES pH 6.5 buffer.

In some embodiments, LNPs comprising an ionizable cationic lipid described herein, prepared and characterized using methods described herein, show average diameter change upon targeting antibody insertion of less than 1, 2, 3, 4, 5, 6, 7, 8, 9, 10, 15, 20, 25, 30, 35, or 40%. In some embodiments, LNPs comprising an ionizable cationic lipid described herein, prepared and characterized using methods described herein, show average diameter change upon targeting antibody insertion of less than 15%. In some embodiments, the diameter change upon targeting antibody insertion is measured in pH 6.5 MES using a 37° C. incubation for 4 hours.

In some embodiments, LNPs comprising an ionizable cationic lipid described herein, prepared and characterized using methods described herein, have average LNP diameter of less than 50, 60, 70, 80, 90, 100, 110, 120, 130, 140, 150, 160, 170, 180, 190, or 200 nm. In some embodiments, LNPs comprising an ionizable cationic lipid described herein, prepared and characterized using methods described herein, have average LNP diameter of less than 100 nm.

Alternatively or in addition, the LNP compositions may have a polydispersity index in a range from 0.05-1, 0.05-0.75, 0.05-0.5, 0.05-0.4, 0.05-0.3, 0.05-0.2, 0.08-1, 0.08-0.75, 0.08-0.5, 0.08-0.4, 0.08-0.3, 0.08-0.2, 0.1-1, 0.1-0.75, 0.1-0.5, 0.1-0.4, 0.1-0.3, 0.1-0.2. In certain embodiments, the polydispersity index is in the range of 0.1-0.25, 0.1-0.2, 0.1-0.19, 0.1-0.18, 0.1-0.17, 0.1-0.16, or 0.1-0.15.

In some embodiments, the LNP compositions or LNPs comprising an ionizable cationic lipid described herein, prepared and characterized using methods described herein, have polydispersity of less than 0.4, 0.3, 0.25, 0.2, 0.15, 0.1, or 0.05. In some embodiments, LNPs comprising an ionizable cationic lipid described herein, prepared and characterized using methods described herein, have polydispersity of less than 0.25.

Alternatively or in addition, the LNP compositions may have a zeta potential of about-30 mV to about +30 mV. In certain embodiments, the LNP composition has a zeta potential of about −10 mV to about +20 mV. The zeta potential may vary as a function of pH. As a result, in certain embodiments, the LNP compositions may have a zeta potential of about 0 m V to about +30 m V or about +10 m V to +30 m V or about +20 m V to about +30 mV at pH 5.5 or pH 5, and/or a zeta potential of about −30 m V to about +5 mV or about −20 m V to about +15 m V at pH 7.4.

In some embodiments, the LNP compositions or LNPs comprising an ionizable cationic lipid described herein, prepared and characterized using methods described herein, have Zeta Potential at pH 7.4 greater than −10, −9, −8, −7, −6, −5.5, −5, −4.5, −4, −3.5, −3, −2.5, −2, −1.5, −1, or −0.5 mV. In some embodiments, the LNP compositions LNPs comprising an ionizable cationic lipid described herein, prepared and characterized using methods described herein, have Zeta Potential at pH 7.4 greater than −10 mV. In some embodiments, the LNP compositions LNPs comprising an ionizable cationic lipid described herein, prepared and characterized using methods described herein, have Zeta Potential at pH 7.4 greater than −1 mV. In some embodiments, the LNP compositions LNPs comprising an ionizable cationic lipid described herein, prepared and characterized using methods described herein, have Zeta Potential at pH 5.5 greater than −1, 0, 1, 2, 3, 4, 4.5, 5, 7.5, 10, 12.5, 15, 17.5, 20, 22.5, or 25 mV. In some embodiments, the LNP compositions LNPs comprising an ionizable cationic lipid described herein, prepared and characterized using methods described herein, have Zeta Potential at pH 5.5 greater than 5 mV. In some embodiments, the LNP compositions LNPs comprising an ionizable cationic lipid described herein, prepared and characterized using methods described herein, have Zeta Potential at pH 5.5 greater than 15 mV.

### Selective Organ Delivery

In some embodiments, the LNP described herein has high liver avoidance. In some embodiments, the LNP comprises Lipid 1, Lipid 2, Lipid 4, Lipid 6, Lipid 7, or Lipid 28, or a salt of any of the foregoing, or any combination of the foregoing. In some embodiments, the LNP comprises Lipid 28, Lipid 6, Lipid 12, Lipid 1, or Lipid 7, or a salt of any of the foregoing, or any combination of the foregoing. In some embodiments, the LNP comprising Lipid 1, Lipid 2, Lipid 4, Lipid 6, Lipid 7, or Lipid 28, or a salt of any of the foregoing, or any combination of the foregoing, has high liver avoidance (e.g., compared to the LNP comprising Lipid C). In some embodiments, the LNP comprising Lipid 28, Lipid 6, Lipid 12, Lipid 1, or Lipid 7, or a salt of any of the foregoing, or any combination of the foregoing, has high liver avoidance (e.g., compared to the LNP comprising Lipid C). In some embodiments, the LNP comprising a lipid of Formula (I), or a salt thereof, or any combination of the foregoing, where R^{c3} is

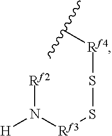

has high liver avoidance. In some embodiments, the LNP comprising a lipid of Formula (I), or a salt thereof, or any combination of the foregoing, where R^{a3} and R^{b3} are each independently

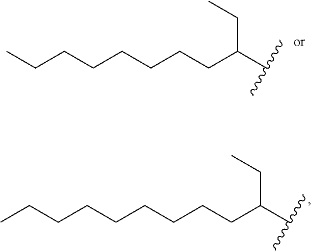 [SMILES: *C(CC)CCCCCCCC ; *C(CC)CCCCCCCCC]

has high liver avoidance.

In some embodiments, liver avoidance is measured with imaging (e.g., ex vivo luciferase imaging). In some embodiments, liver avoidance is measured as a non-liver/liver ratio. In some embodiments, the non-liver/liver ratio is the level of LNP accumulation, cargo delivery, or cargo expression in a non-liver organ (e.g., spleen) relative to that in the liver. In some embodiments, liver avoidance is measured certain period of time (e.g., 24 hours) after dosing the subject with the LNP. In some embodiments, the non-liver/liver ratio is greater than about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1000. In some embodiments, the non-liver/liver ratio is more than about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1000 times higher than the non-liver/liver ratio of the LNP comprising Lipid C. In some embodiments, the LNP comprises Lipid 28, and the non-liver/liver ratio is greater than about 500. In some embodiments, the LNP comprises Lipid 28, and the non-liver/liver ratio is more than about 500 times higher than the non-liver/liver ratio of the LNP comprising Lipid C. In some embodiments, the non-liver/liver ratio is about 1, 2, 3, 4, 5, 6, 7, 8, 9, 10, 11, 12, 13, 14, 15, 16, 17, 18, 19, or 20. In some embodiments, the non-liver/liver ratio is more than about 1, 2, 3, 4, 5, 6, 7, 8, 9, 10, 11, 12, 13, 14, 15, 16, 17, 18, 19, or 20 times higher than the non-liver/liver ratio of the LNP comprising Lipid C.

In some embodiments, the LNP described herein has high liver targeting. In some embodiments, the LNP comprises Lipid 9 or Lipid 10 of Table 1, or a salt of any of the foregoing, or any combination of the foregoing. In some embodiments, the LNP comprising Lipid 9 or Lipid 10, or a salt of any of the foregoing, or any combination of the foregoing, has high liver targeting (e.g., compared to the LNP comprising Lipid C). In some embodiments, the LNP comprising a lipid of Formula (I), or a salt thereof, or any combination of the foregoing, where R^{a3} and R^{b3} are each independently

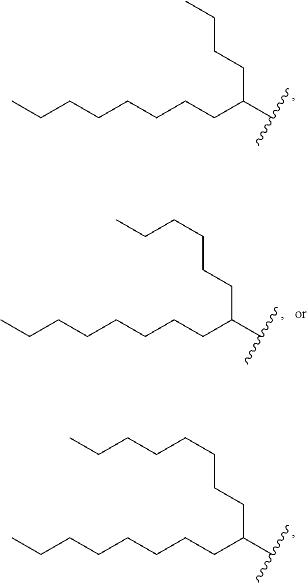 [SMILES: *C(CCCC)CCCCCCCC ; *C(CCCCCC)CCCCCCCC ; *C(CCCCCCCC)CCCCCCCC]

has high liver targeting. In some embodiments, the LNP comprising a lipid of Formula (I), or a salt thereof, or any combination of the foregoing, where at least one of R^{a3} and R^{b3} is

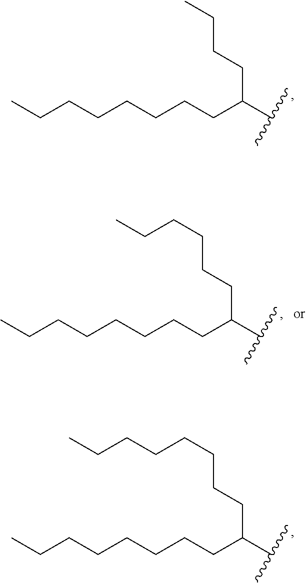 [SMILES: *C(CCCC)CCCCCCCC ; *C(CCCCCC)CCCCCCCC ; *C(CCCCCCCC)CCCCCCCC]

has high liver targeting.

In some embodiments, liver targeting is measured with imaging (e.g., ex vivo luciferase imaging). In some embodiments, liver targeting is measured as a liver/non-liver ratio. In some embodiments, the liver/non-liver ratio is the level of LNP accumulation, cargo delivery, or cargo expression in the liver relative to that in a non-liver organ (e.g., spleen). In some embodiments, liver targeting is measured certain period of time (e.g., 24 hours) after dosing the subject with the LNP. In some embodiments, the liver/non-liver ratio is greater than about 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20. In some embodiments, the liver/non-liver ratio is more than about 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, or 20 times higher than the liver/non-liver ratio of the LNP comprising Lipid C. In some embodiments, the liver/non-liver ratio is more than about 1.5 to 2 times higher than the liver/non-liver ratio of the LNP comprising Lipid C.

### V. Payloads

The LNP compositions may comprise an agent, for example, a nucleic acid molecule for delivery to a cell (e.g., an immune cell) or tissue, for example, a cell (e.g., an immune cell) or tissue in a subject.

The LNP compositions of the present invention may include a nucleic acid, for example, a DNA or RNA, such as an mRNA, IRNA, microRNA, siRNA, gRNA (guide RNA), circRNA (circular RNA), ribozymes, decoy RNA or dicer substrate siRNA. It is contemplated that nucleic acids can comprise naturally occurring components, such as, naturally occurring bases, sugars or linkage groups (e.g., phosphodiester linkage groups) or may comprise non-naturally occurring components or modifications, (e.g., thioester linkage groups). For example, the nucleic acid can be synthesized to comprise base, sugar, and/or linker modifications known to those skilled in the art. Furthermore, the nucleic acids can be linear or circular, or have any desired configuration. The LNP compositions can include multiple nucleic acid molecules, for example, multiple RNA molecules, which can be the same or different.

In certain embodiments, the payload is an mRNA. In certain embodiments, a particular LNP composition may comprise a number of mRNA molecules that can be the same or different. In certain embodiments, one or more LNP compositions including one or more different mRNAs may be combined, and/or simultaneously contacted, with a cell. It is contemplated that an mRNA may include one or more of a stem loop, a chain terminating nucleoside, a polyA sequence, a polyadenylation signal, and/or a 5′ cap structure. The mRNA may encode a receptor, such as a chimeric antigen receptor (CAR), for use in for example, an immune disorder, inflammatory disorder or cancer. In addition, the mRNA may encode an antigen for use in a therapeutic or prophylactic vaccine, for example, for treating or preventing an infection by a pathogen, for example, a microbial or viral pathogen, or for reducing or ameliorating the side effects caused directly or indirectly by such an infection.

In certain embodiments, the LNP composition may include one or more other components including, but not limited to, one or more pharmaceutically acceptable excipients, small hydrophobic molecules, therapeutic agents, carbohydrates, polymers, permeability enhancing molecules, and surface altering agents.

In some embodiments, the wt/wt ratio of the lipid component to the payload (e.g., mRNA) in the resulting LNP composition is from about 1:1 to about 50:1. In certain embodiments, the wt/wt ratio of the lipid component to the payload (e.g., mRNA) in the resulting composition is from about 5:1 to about 50:1. In certain embodiments, the wt/wt ratio is from about 5:1 to about 40:1. In certain embodiments, the wt/wt ratio is from about 10:1 to about 40:1. In certain embodiments, the wt/wt ratio is from about 15:1 to about 25:1.

In certain embodiments, the encapsulation efficiency of the payload (e.g., mRNA) in the lipid nanoparticles is at least 50%. In certain embodiments, the encapsulation efficiency is at least 80%, at least 90% or greater than 90%.

In some embodiments, LNPs comprising an ionizable cationic lipid described herein, prepared and characterized using methods described herein, exhibit encapsulation efficiency of greater than 50, 55, 60, 65, 70, 75, 80, 82.5, 85, 87.5, 90, 92.5, 95, 97.5, or 99%. In some embodiments, LNPs comprising an ionizable cationic lipid described herein, prepared and characterized using methods described herein, exhibit encapsulation efficiency of greater than 87.5%. In some embodiments, LNPs comprising an ionizable cationic lipid described herein, prepared and characterized using methods described herein, exhibit dye accessible RNA of less than 50, 45, 40, 35, 30, 25, 20, 17.5, 15, 12.5, 10, 7.5, 5, 2.5, or 1%. In some embodiments, LNPs comprising an ionizable cationic lipid described herein, prepared and characterized using methods described herein, exhibit dye accessible RNA of less than 12.5%.

In some embodiments, LNPs comprising an ionizable cationic lipid described herein, prepared and characterized using methods described herein, exhibit total mRNA recovery of greater than 50, 55, 60, 65, 70, 75, 80, 85, 90, or 95%. In some embodiments, LNPs comprising an ionizable cationic lipid described herein, prepared and characterized using methods described herein, exhibit total mRNA recovery of greater than 80%.

### RNA Payload

In certain embodiments, the RNA payload is an mRNA, IRNA, microRNA, or siRNA payload.

In certain embodiments, the lipid nanoparticle compositions are optimized for the delivery of RNA, e.g., mRNA, to a target cell for translation within the cell. An mRNA may be a naturally or non-naturally occurring mRNA. An mRNA may include one or more modified nucleobases, nucleosides, or nucleotides.

The nucleobases may be selected from the non-limiting group consisting of adenine, guanine, uracil, cytosine, 7-methylguanine, 5-methylcytosine, 5-hydroxymethylcytosine, thymine, pseudouracil, dihydrouracil, N1-methylpseudouracil, hypoxanthine, and xanthine. In some embodiments, nucleobase is N1-methylpseudouracil.

A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications.

A nucleotide of an mRNA is a compound comprising a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation, and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein.

An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine. In certain embodiments, one or more or all uridine bases may be N1-methylpseudouridines.

In certain embodiments, an mRNA may include a 5′ cap structure, a chain terminating nucleotide, a stem loop, a polyA sequence, and/or a polyadenylation signal.

A cap structure or cap species is a compound including two nucleoside moieties joined by a linker and may be selected from a naturally occurring cap, a non-naturally occurring cap or a cap analog. A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G (5′) ppp (5′) G, commonly written as m7GpppG. A cap species may also be an anti-reverse cap analog. A non-limiting list of possible cap species includes m7GpppG, m7Gpppm7G, m73′dGpppG, m7Gpppm7G, m73′dGpppG, and m27 02′GppppG.

Alternatively or in addition, an mRNA may include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

Alternatively or in addition, an mRNA may include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

Alternatively or in addition, an mRNA may include a polyA sequence and/or polyadenylation signal. A polyA sequence may be comprised entirely or mostly of adenine nucleotides or analogs or derivatives thereof. A poly A sequence may be a tail located adjacent to a 3′ untranslated region of an mRNA.

An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell. In some embodiments, the mRNA may encode an antibody, enzyme, growth factor, hormone, cytokine, viral protein (e.g., a viral capsid protein), antigen, vaccine, or receptor. In some embodiments, the mRNA may encode an engineered receptor such as a CAR or an antigen for use in a therapeutic vaccine (e.g., a cancer vaccine) or a prophylactic vaccine (e.g., a vaccine for minimizing the risk or severity of an infection by a microbial or viral pathogen). In some embodiments, the mRNA encodes a polypeptide capable of regulating immune response in the immune cell. In some embodiments, the mRNA encodes a polypeptide capable of reprogramming the immune cell. In some embodiments, the mRNA encodes a synthetic T cell receptor (synTCR) or a Chimeric Antigen Receptor (CAR).

A lipid composition may be designed for one or more specific applications or targets. For example, an LNP composition may be designed to deliver mRNA to a particular cell, tissue, organ, or system or group thereof in a mammal's body, such as the renal system. Physiochemical properties of LNP compositions may be altered in order to increase selectivity for particular target site within a subject. For instance, particle sizes may be adjusted based on the fenestration sizes of different organs. The mRNA included in an LNP composition may also depend on the desired delivery target or targets. For example, an mRNA may be selected for a particular indication, condition, disease, or disorder and/or for delivery to a particular cell, tissue, organ, or system or group thereof (e.g., localized or specific delivery).

The amount of mRNA in a lipid composition may depend on the size, sequence, and other characteristics of the mRNA. The amount of mRNA in an LNP may also depend on the size, composition, desired target, and other characteristics of the LNP composition. The relative amounts of mRNA and other elements (e.g., lipids) may also vary. The amount of mRNA in an LNP composition may, for example, be measured using absorption spectroscopy (e.g., ultraviolet-visible spectroscopy).

In some embodiments, the one or more mRNAs, lipids, and polymers and amounts thereof may be selected to provide a specific N:P ratio (the ratio of positively-chargeable lipid or polymer amine (N=nitrogen) groups to negatively-charged nucleic acid phosphate (P) groups). The N:P ratio of the composition refers to the molar ratio of nitrogen atoms in one or more lipids to the number of phosphate groups in an mRNA. In general, a lower N:P ratio is preferred. A N:P ratio may be dependent on a specific lipid and its pKa. In certain embodiments, the mRNA and LNP composition, and/or their relative amounts may be selected to provide an N:P ratio from about 1:1 to about 30:1, or from about 1:1 to about 20:1. In certain embodiments, the N:P ratio can be, for example, 1:1, 2:1, 3:1, 4:1, 5:1, 6:1, 7:1, or 8:1. In certain embodiments, the N:P ratio may be from about 2:1 to about 5:1. In certain embodiments, the N:P ratio may be about 4:1. In other embodiments, the N:P ratio is from about 4:1 to about 8:1. For example, the N:P ratio may be about 4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 6.0:1, about 6.5:1, or about 7.0:1.

The amount of mRNA in a nanoparticle composition may depend on the size, sequence, and other characteristics of the mRNA. The amount of mRNA in a nanoparticle composition may also depend on the size, composition, desired target, and other characteristics of the nanoparticle composition. The relative amounts of mRNA and other elements (e.g., lipids) may also vary. In some embodiments, the wt/wt ratio of the lipid component to an mRNA in a nanoparticle composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of the lipid component to an mRNA may be from about 10:1 to about 40:1. The amount of mRNA in a nanoparticle composition may, for example, be measured using absorption spectroscopy (e.g., ultraviolet-visible spectroscopy).

The efficiency of encapsulation of an mRNA describes the amount of mRNA that is encapsulated or otherwise associated with a lipid composition after preparation, relative to the initial amount provided. The encapsulation efficiency is desirably high (e.g., close to 100%). The encapsulation efficiency may be measured, for example, by comparing the amount of mRNA in a solution comprising the lipid composition before and after breaking up the LNP composition with one or more organic solvents or detergents. Fluorescence may be used to measure the amount of free mRNA in a solution. For the LNP compositions of the invention, the encapsulation efficiency of an mRNA may be at least 50%, for example 50%, 55%, 60%, 65%, 70%, 75%, 80%, 85%, 90%, 91%, 92%, 93%, 94%, 95%, 96%, 97%, 98%, 99%, or 100%. In certain embodiments, the encapsulation efficiency may be at least 80%.

### VI. Formulation and Mode of Delivery

LNP compositions of the invention may be formulated in whole or in part as a pharmaceutical composition. The pharmaceutical compositions may further include one or more pharmaceutically acceptable excipients or accessory ingredients such as those described herein. General guidelines for the formulation and manufacture of pharmaceutical compositions and agents are available, for example, in Remington's (2006) supra. Conventional excipients and accessory ingredients may be used in any pharmaceutical composition of the invention, except insofar as any conventional excipient or accessory ingredient may be incompatible with one or more components of an LNP composition of the invention. An excipient or accessory ingredient may be incompatible with a component of an LNP composition if its combination with the component may result in any undesirable biological effect or otherwise deleterious effect.

In some embodiments, one or more excipients or accessory ingredients may make up greater than 50% of the total mass or volume of a pharmaceutical composition including an LNP composition of the invention. For example, the one or more excipients or accessory ingredients may make up 30%, 40%, 50%, 60%, 70%, 80%, 90%, or more of a pharmaceutical composition. In certain embodiments, the excipient is approved for use in humans and for veterinary use, for example, by United States Food and Drug Administration. In certain embodiments, the excipient is pharmaceutical grade. In certain embodiments, an excipient meets the standards of the United States Pharmacopoeia (USP), the European Pharmacopoeia (EP), the British Pharmacopoeia, and/or the International Pharmacopoeia.

Relative amounts of the one or more lipids or LNPs, one or more pharmaceutically acceptable excipients, and/or any additional ingredients in a pharmaceutical composition will vary, depending upon the identity, size, and/or condition of the subject treated and further depending upon the route by which the composition is to be administered.

Lipid compositions and/or pharmaceutical compositions including one or more LNP compositions may be administered to any subject, including a human patient that may benefit from a therapeutic effect provided by the delivery of a nucleic acid, e.g., an RNA (e.g., mRNA, RNA or siRNA) to one or more particular cells, tissues, organs, or systems or groups thereof, such as the renal system. Although the descriptions provided herein of LNP compositions and pharmaceutical compositions including LNP compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is understood.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., the payload).

Pharmaceutical compositions of the invention may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, com, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

### Other Components

In addition, it is contemplated that the pharmaceutical compositions may include one or more components in addition to those described hereinabove.

The pharmaceutical compositions may also include one or more permeability enhancer molecules, carbohydrates, polymers, therapeutic agents, surface altering agents, or other components. A permeability enhancer molecule may be a molecule described, for example, in U.S. patent application publication No. 2005/0222064. Carbohydrates may include simple sugars (e.g., glucose) and polysaccharides (e.g., glycogen and derivatives and analogs thereof).

The pharmaceutical compositions may also comprise a surface altering agent, including for example, anionic proteins (e.g., bovine serum albumin), surfactants (e.g., cationic surfactants such as dimethyldioctadecyl-ammonium bromide), sugars or sugar derivatives (e.g., cyclodextrin), polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin B4, dornase alfa, neltenexine, and erdosteine), and DNases (e.g., rhDNase). A surface altering agent may be disposed within and/or upon the surface of a composition described herein.

In addition to these components, a pharmaceutical composition comprising an LNP composition of the invention may include any substance useful in pharmaceutical compositions. For example, the pharmaceutical composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see, e.g., Remington's (2006) supra).

Dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In certain embodiments, the lipid nanoparticle compositions and formulations thereof are adapted for administration intravenously, intramuscularly, intradermally, subcutaneously, intra-arterially, intra-tumor, or by inhalation. In certain embodiments, a dose of about 0.001 mg/kg to about 10 mg/kg is administered to a subject. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

The specific therapeutically effective, prophylactically effective, or otherwise appropriate dose level (e.g., for imaging) for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

### VII. Methods

The present disclosure provides methods of delivering a payload to a target cell or tissue, for example, a target cell or tissue in a subject, and LNPs or pharmaceutical compositions comprising the LNPs for use in such methods. Any disclosure herein of a method of, e.g., treating a disease or disorder or, e.g., delivering a nucleic acid to a cell or, e.g., producing a polypeptide of interest in a cell should be interpreted also as a disclosure of an LNP or pharmaceutical composition comprising said LNP for use in such methods.

In certain embodiments, the invention provides a method of producing a polypeptide of interest (e.g., a protein of interest) in a mammalian cell, and LNPs or pharmaceutical compositions comprising the LNPs for use in such methods. Methods of producing polypeptides in such a cell involve contacting a cell with an LNP composition comprising an RNA of interest (e.g., an mRNA encoding the polypeptide of interest (e.g., a protein of interest). Upon contacting the cell with the LNP composition, the mRNA may be taken up and translated in the cell to produce the polypeptide of interest.

In general, the step of contacting a mammalian cell with an LNP composition including an mRNA encoding a polypeptide of interest may be performed in vivo, ex vivo, or in vitro. The amount of an LNP composition contacted with a cell, and/or the amount of mRNA therein, may depend on the type of cell or tissue being contacted, the means of administration, the physiochemical characteristics of the LNP composition and the mRNA (e.g., size, charge, and chemical composition) therein, and other factors. In general, an effective amount of the LNP composition will allow for efficient polypeptide production in the cell. Metrics for efficiency may include polypeptide translation (indicated by polypeptide expression), level of mRNA degradation, and immune response indicators.

The step of contacting an LNP composition including an mRNA with a cell may involve or cause transfection where the LNP composition may fuse with the membrane of cell to permit the delivery of the mRNA into the cell. Upon introduction into the cytoplasm of the cell, the mRNA is then translated into a protein or peptide via the protein synthesis machinery within the cytoplasm of the cell.

In certain embodiments, the LNP compositions described herein may be used to deliver therapeutic or prophylactic agents to a subject. For example, an mRNA included in an LNP composition may encode a polypeptide and produce the therapeutic or prophylactic polypeptide upon contacting and/or entry (e.g., transfection) into a cell. In certain embodiments, an mRNA included in an LNP composition of the invention may encode a polypeptide that may improve or increase the immunity of a subject.

In certain embodiments, contacting a cell with an LNP composition including an mRNA may reduce the innate immune response of a cell to an exogenous nucleic acid. A cell may be contacted with a first LNP composition including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times.

Additionally, efficiency of polypeptide production in the cell may be optionally determined, and the cell may be re-contacted with the first and/or second composition repeatedly until a target protein production efficiency is achieved.

The present disclosure provides methods of delivering a nucleic acid (e.g., an mRNA) to a mammalian cell or tissue, for example, a mammalian cell or tissue in a subject. Delivery of an mRNA to such a cell or tissue involves administering an LNP composition including the mRNA to a subject, for example, by injection, e.g., via intramuscular injection or intravascular delivery into the subject. After administration, the LNP can target and/or contact a cell, for example, an immune cell, such as a T-cell. Upon contacting the cell with the LNP composition, a translatable mRNA may be translated in the cell to produce a polypeptide of interest.

In certain embodiments, an LNP composition of the invention may target a particular type or class of cells. This targeting may be facilitated using the lipids described herein to form LNPs, which may also include a targeting group for targeting cells of interest. In certain, embodiments, specific delivery may result in a greater than 2 fold, 5 fold, 10 fold, 15 fold, or 20 fold increase in the amount of mRNA to the targeted destination (e.g., cells that express or express at high levels the receptor of interest which binds to the cell targeting group of the LNPs) as compared to another destinations (e.g., cells that either do not express or only express at low levels the receptor of interest).

LNP compositions of the invention may be useful for treating a disease, disorder, or condition characterized by missing or aberrant protein or polypeptide activity. Upon delivery of an mRNA encoding the missing or aberrant polypeptide to a cell, translation of the mRNA may produce the polypeptide, thereby reducing or eliminating an issue caused by the absence of or aberrant activity caused by the polypeptide. Because translation may occur rapidly, the methods and compositions of the invention may be useful in the treatment of acute diseases, disorders, or conditions such as sepsis, stroke, and myocardial infarction. An mRNA included in an LNP composition of the invention may also be capable of altering the rate of transcription of a given species, thereby affecting gene expression.

Diseases, disorders, and/or conditions characterized by dysfunctional or aberrant protein or polypeptide activity for which a composition of the invention may be administered include, but are not limited to, cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis. The present disclosure provides a method for treating such diseases, disorders, and/or conditions in a subject by administering an LNP composition including an mRNA and a lipid component including KL10, a phospholipid (optionally unsaturated), a PEG lipid, and a structural lipid, wherein the mRNA encodes a polypeptide that antagonizes or otherwise overcomes an aberrant protein activity present in the cell of the subject.

The therapeutic and/or prophylactic compositions described herein may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject, the purpose of the administration, the particular composition, the mode of administration, and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

A LNP composition including one or more mRNAs may be administered by a variety of routes, for example, orally, intravenously, intramuscularly, intra-arterially, intramedullary, intrathecally, subcutaneously, intraventricularly, trans- or intra-dermally, intradermally, rectally, intravaginally, intraperitoneally, topically, mucosally, nasally, intratumorally. In certain embodiments, an LNP composition may be administered intravenously, intramuscularly, intradermally, intra-arterially, intratumorally, or subcutaneously. However, the present disclosure encompasses the delivery of LNP compositions of the invention by any appropriate route taking into consideration likely advances in the sciences of drug delivery. In general, the most appropriate route of administration will depend upon a variety of factors including the nature of the LNP composition including one or more mRNAs (e.g., its stability in various bodily environments such as the bloodstream and gastrointestinal tract), the condition of the patient (e.g., whether the patient is able to tolerate particular routes of administration), etc.

LNP compositions including one or more mRNAs may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more LNP compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

In some embodiments, no more than 1%, no more than 2%, no more than 3%, no more than 4%, no more than 5%, no more than 6%, no more than 7%, no more than 8%, no more than 9%, no more than 10%, no more than 15%, no more than 20%, no more than 25%, no more than 30%, no more than 35%, no more than 40%, no more than 45%, or no more than 50% of cells that are not meant to be the destination of the delivery are transfected by the LNP. In some embodiments, the cells that are not meant to be the destination of the delivery are subject's non-immune cells. In some embodiments, the cells that are not meant to be the destination of the delivery are cells not targeted by the method. In some embodiments, the cells that are not meant to be the destination of the delivery are subject's cells not targeted by the method.

In some embodiments, the half-life of the nucleic acid delivered by the LNP described herein to the cell or a polypeptide encoded by the nucleic acid delivered by the LNP and expressed in the cell is at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, or at least 5 times longer than the half-life of the nucleic acid delivered by a reference LNP to the cells or a polypeptide encoded by the nucleic acid delivered by the reference LNP and expressed in the cell.

In some embodiments, the composition of the LNP differs from the composition of the reference LNP in the type of ionizable cationic lipid, relative amount of ionizable cationic lipid, length of the lipid anchor in PEG lipid, back bone or head group of the PEG lipid, relative amount of PEG lipid, or type of cell targeting group, or any combination thereof. In some embodiments, the composition of the LNP differs from the composition of the reference LNP only in the type of ionizable cationic lipid. In some embodiments, the composition of the LNP differs from the composition of the reference LNP only in the amount of PEG lipid. In some embodiments, the reference LNP comprises cationic Lipid DLin-KC3-DMA, but otherwise as the same as a tested LNP. In some embodiments, the reference LNP comprises cationic Lipid DLin-KC2-DMA, but otherwise as the same as a tested LNP. In some embodiments, the reference LNP comprises cationic Lipid ALC-0315, but otherwise as the same as a tested LNP. In some embodiments, the reference LNP comprises cationic Lipid SM-102, but otherwise as the same as a tested LNP. In some embodiments, PEG lipid is a free PEG-lipid.

In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the target cells (e.g., immune cells) are transfected by the LNP. In some embodiments, the target cells are subject's immune cells. In some embodiments, the immune cells are immune cells targeted by the method. In some embodiments, the immune cells are subject's immune cells targeted by the method. In some embodiments, the immune cells are macrophages, for instance M2a macrophages, M2b macrophages, and/or M2c macrophages. In some embodiments, the immune cells are B cells. In some embodiments, the immune cells are NK cells. In some embodiments, the immune cells are T cells, for example CD4+ T cells and/or CD8+ T cells. In some embodiments, the immune cells are NK cells and T cells, for example NK cells and CD4+ T cells and/or CD8+ T cells. In some embodiments, the immune cells are monocytes. In some embodiments, the immune cells are dendritic cells.

In some embodiments, the expression level of the nucleic acid delivered by the LNP is at least at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 2 times, at least 3 times, at least 4 times, at least 5 times, at least 6 times, at least 7 times, at least 8 times, at least 9 times, or at least 10 times higher than the expression level of the nucleic acid delivered by a reference LNP. In some embodiments, the expression level is measured and compared with a method described herein. In some embodiments, the expression level is measured by the ratio of cells expressing the encoded polypeptide. In some embodiments, the expression level is measured with FACS. In some embodiments, the expression level is measured by the average amount of the encoded polypeptide expressed in cells. In some embodiments, the expression level is measured as mean fluorescence intensity. In some embodiments, the expression level is measured by the amount of the encoded polypeptide or other materials secreted by cells.

In another aspect, provided herein are methods of targeting the delivery of a nucleic acid to an immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the compound of the following formula: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, an aspect of the disclosure relates to an LNP or a pharmaceutical composition comprising the LNP, as disclosed herein, for use in a method of targeting the delivery of a nucleic acid to an immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP). In some embodiments, the LNP is an LNP as described herein in the present disclosure.

In some embodiments, the LNP provides at least one of the following benefits:
(i) increased specificity of targeted delivery to the immune cell compared to a reference LNP;
(ii) increased half-life of the nucleic acid or a polypeptide encoded by the nucleic acid in the immune cell compared to a reference LNP;
(iii) increased transfection rate compared to a reference LNP; and
(iv) a low level of dye accessible mRNA (<15%) and high RNA encapsulation efficiencies, wherein at least 80% mRNA was recovered in final formulation relative to the total RNA used in LNP batch preparation.

In some aspect, provided are methods of expressing a polypeptide of interest in a targeted immune cell of a subject. In some embodiments, the method comprises contacting the immune cell with a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition comprising the LNP, as disclosed herein, for use in a method of expressing a polypeptide of interest in a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP; and
(v) a low level of dye accessible mRNA (<15%) and high RNA encapsulation efficiencies, wherein at least 80% mRNA was recovered in final formulation relative to the total RNA used in LNP batch preparation.

In some aspects, provided are methods of modulating cellular function of a target immune cell of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the immune cell. In some embodiments, an aspect of the disclosure relates to an LNP or a pharmaceutical composition comprising the LNP, as disclosed herein, for use in a method of modulating cellular function of a targeted immune cell of a subject. Such a method may be for the treatment of a disease or disorder as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:
(i) increased expression level in the immune cell compared to a reference LNP;
(ii) increased specificity of expression in the immune cell compared to a reference LNP;
(iii) increased half-life of the nucleic acid or a polypeptide encoded by the nucleic acid in the immune cell compared to a reference LNP;
(iv) increased transfection rate compared to a reference LNP;
(v) the LNP can be administered at a lower dose compared to a reference LNP to reach the same biologic effect in the immune cell; and
(vi) a low level of dye accessible mRNA (<15%) and high RNA encapsulation efficiencies, wherein at least 80% mRNA was recovered in final formulation relative to the total RNA used in LNP batch preparation.

In some embodiments, the modulation of cell function comprises reprogramming the immune cells to initiate an immune response. In some embodiments, the modulation of cell function comprises modulating antigen specificity of the immune cell.

In some aspect, provided are methods of treating, ameliorating, or preventing a symptom of a disorder or disease in a subject in need thereof. In some embodiments, the method comprises administering to the subject a lipid nanoparticle (LNP) for delivering a nucleic acid into an immune cell of the subject. In some embodiments, the LNP comprises an ionizable cationic lipid. In some embodiments, the LNP comprises a conjugate comprising the following structure: [Lipid]-[optional linker]-[immune cell targeting group]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises the nucleic acid.

In some embodiments, the nucleic acid modulates the immune response of the immune cell, therefore to treat or ameliorate the symptom. In some embodiments, an aspect of the disclosure relates to an LNP or a pharmaceutical composition comprising the LNP, as disclosed herein, for use in a method of treating, ameliorating, or preventing a symptom of a disorder or disease in a subject in need thereof. A disease or disorder may be as disclosed hereafter. In some embodiments, a method as disclosed herein can comprise contacting in vitro or ex vivo the immune cell of a subject with a lipid nanoparticle (LNP).

In some embodiments, the LNP provides at least one of the following benefits:
(i) increased specificity of delivery of the nucleic acid into the immune cell compared to a reference LNP;
(ii) increased half-life of the nucleic acid or a polypeptide encoded by the nucleic acid in the immune cell compared to a reference LNP;
(iii) increased transfection rate compared to a reference LNP;
(iv) the LNP can be administered at a lower dose compared to a reference LNP to reach the same treatment efficacy;
(v) increased level of gain of function by an immune cell compared to a reference LNP; and (vi) a low level of dye accessible mRNA (<15%) and high RNA encapsulation efficiencies, wherein at least 80% mRNA was recovered in final formulation relative to the total RNA used in LNP batch preparation.

In some embodiments, the disorder is an immune disorder, an inflammatory disorder, or cancer. In some embodiments, the nucleic acid encodes an antigen for use in a therapeutic or prophylactic vaccine for treating or preventing an infection by a pathogen.

In some embodiments, no more than 1%, 2%, 3%, 4%, 5%, 6%, 7%, 8%, 9%, or 10% of non-immune cells are transfected by the LNP. In some embodiments, no more than 1%, 2%, 3%, 4%, 5%, 6%, 7%, 8%, 9%, or 10% of undesired immune cells that are not meant to be the destination of the delivery are transfected by the LNP. In some embodiments, the half-life of the nucleic acid delivered by the LNP to the immune cell or a polypeptide encoded by the nucleic acid delivered by the LNP is at least 5%, 10%, 15%, 20%, 25%, 30%, 40%, 50%, 60%, 70%, 80%, 90%, 100%, 1.5 times, 2 times, 3 times, 4 times, 5 times, 10 times, or longer than the half-life of nucleic acid delivered by a reference LNP to the immune cell or a polypeptide encoded by the nucleic acid delivered by the reference LNP.

In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more immune cells that are meant to be the destination of the delivery are transfected by the LNP.

In some embodiments, expression level of the nucleic acid delivered by the LNP is at least 5%, at least 10%, at least 10%, at least 10%, at least 10%, at least 10%, at least 10%, at least 10%, at least 10%, at least 10%, at least 10%, at least 10%, at least 10%, at least 10%, at least 10%, 1.5 time, 2 times, 3 times, 4 times, 5 times, 10 times, 15 times, 20 times or more higher than expression level of nucleic acid in the same immune cells delivered by a reference LNP.

In some aspects, provided are methods of targeting the delivery of a nucleic acid to an immune cell of a subject. In some embodiments, the method comprises contacting the immune cell with a lipid nanoparticle (LNP) provided herein. In some embodiments, the method is for targeting NK cells. In some embodiments, the immune cell targeting group binds to CD56. In some embodiments, the method is for targeting both T cells and NK cells simultaneously. In some embodiments, the immune cell targeting group binds to CD7, CD8, or both CD7 and CD8. In some embodiments, the method is for targeting both CD4+ and CD8+ T cells simultaneously. In some embodiments, the immune cell targeting group comprises a polypeptide that binds to CD3 or CD7.

In some aspects, provided are methods of expressing a polypeptide of interest in a targeted immune cell of a subject. In some embodiments, the method comprises contacting the immune cell with a lipid nanoparticle (LNP) provided herein.

In some aspect, provided are method of modulating cellular function of a target immune cell of a subject. In some embodiments, the method comprises administering to the subject a lipid nanoparticle (LNP) provided herein.

In some aspects, provided are method of treating, ameliorating, or preventing a symptom of a disorder or disease in a subject in need thereof. In some embodiments, the method comprises administering to the subject a lipid nanoparticle (LNP) provided herein.

In some aspects, provided are methods of treating a disease or disorder related to CD8 in a subject. In some embodiments, the method comprises administering a pharmaceutical composition described herein to the subject. In some embodiments, the disease or disorder is cancer.

LNPs disclosed in the present disclosure and as claimed are suitable for the methods described above.

### VIII. Kits for Use in Medical Applications

Another aspect of the invention provides a kit for treating a disorder. The kit comprises: an ionizable cationic lipid, a lipid-cell targeting group conjugate (e.g., a lipid-immune cell targeting group conjugate), a lipid nanoparticle composition comprising an ionizable cationic lipid and/or a lipid-cell targeting group conjugate with or without an encapsulated payload (e.g., an mRNA), and instructions for treating a medical disorder, such as, cancer or a microbial or viral infection.

### ENUMERATED EMBODIMENTS

The following enumerated embodiments are representative of some aspects of the invention.

### Enumerated Embodiments Set A

The embodiment numbers refenced in this set refers to those within set A. For example, “embodiment 1” recited in embodiment A2 refers to embodiment A1.
A1. A compound of Formula (I-P1):

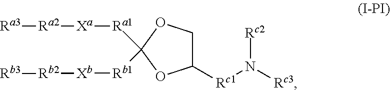

or a salt thereof, wherein:
   R^{a1} and R^{b1} are each independently C₁₋₁₂ alkylene;
   X^{a} and X^{b} are each independently —C(O)O—* or —OC(O)—*, wherein * indicates the point of attachment to R^{a1} or Rbi;
   R^{a2} and R^{b2} are each independently a bond or C₁₋₃ alkylene;
   R^{a3} is

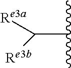

and R^{b3} is

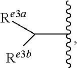

wherein R^{a3a}, R^{a3b}, R^{b3a}, and R^{b3b} are each independently H or C₁₋₁₂ alkyl optionally substituted with heterocylyl;
R^{c1} is C₁₋₆ alkylene;
R^{c2} is C₁₋₆ alkyl; and
R^{c3} is C₁₋₆ alkyl or

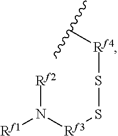

wherein:
   R^{f1} is H or

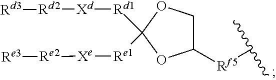

R^{f2} is C₁₋₆ alkyl;
R^{f3} and R^{f4} are each independently C₁₋₆ alkylene; and
R^{d1} and R^{e1} are each independently C₁₋₁₂ alkylene;
X^{d} and X^{e} are each independently —C(O)O—* or —OC(O)—*, wherein * indicates the point of attachment to R^{d1} or R^{e1}.
R^{d2} and R^{e2} are each independently a bond or C₁₋₃ alkylene; and
R^{d3} is

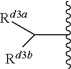

and R^{e3} is

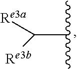

wherein R^{d3a}, R^{d3b}, R^{e3a}, and R^{e35} are each independently H or C₁₋₁₂ alkyl optionally substituted with heterocylyl;
with the proviso that when R^{c1} is —CH₂— and R^{c2} and R^{c3} are each methyl, then none of R^{a3a}, R^{a3b}, R^{b3a}, and R^{b3b} is H; when R^{c1} is —(CH₂)₂— and R^{c2} and R^{c3} are each methyl, then R^{b3a} is not H and R^{b3b} is ethyl; when R^{c1} is —(CH₂)₂— and R^{c2}, R^{c3}, or both R^{c2} and R^{c3} are not methyl, then none of R^{a3a}, R^{a3b}, R^{b3a}, and R^{b3b} is H; when R^{c1} is —(CH₂)₃—, R^{c2} and R^{c3} are each methyl, and one of R^{a3a}, R^{a3b}, R^{b3a}, and R^{b3b} is H, then at least one of R^{a3a}, R^{a3b}, R^{b3a}, and R^{b3b} that is not H is substituted with a heterocylyl; and when R^{c1} is —(CH₂)₄—, R^{c2} and R^{c3} are each methyl, then none of R^{a3a}, R^{a3b}, R^{b3a}, and R^{b3b} is H.
A2. The compound of embodiment 1, or a salt thereof, wherein R^{a1} and R^{b1} are each independently a linear C₁₋₁₂ alkylene.
A3. The compound of embodiment 1 or 2, or a salt thereof, wherein R^{a1} and R^{b1} are each independently C5-10 alkylene.
A4. The compound of embodiment 1, or a salt thereof, wherein R^{a1} and R^{b1} are each —(CH₂)₇—.
A5. The compound of any one of embodiments 1-4, or a salt thereof, wherein X^{a} and X^{b} are each —C(O)O—*.
A6. The compound of any one of embodiments 1-4, or a salt thereof, wherein X^{a} and X^{b} are each —OC(O)—*.
A7. The compound of any one of embodiments 1-6, or a salt thereof, wherein R^{a2} and R^{b2} are each a bond.
A8. The compound of any one of embodiments 1-6, or a salt thereof, wherein R^{a2} and R^{b2} are each —CH₂—.
A9. The compound of any one of embodiments 1-8, or a salt thereof, wherein no more than one of R^{a3a}, R^{a3b}, R^{b3a}, and R^{b3b} is H.
A10. The compound of any one of embodiments 1-9, or a salt thereof, wherein R^{a3a}, R^{a3b}, R^{b3} and R^{b3b} are each independently a linear C₁₋₁₂ alkyl.
A11. The compound of any one of embodiments 1-10, or a salt thereof, wherein R^{a3a}, R^{a3b} R^{b3a}, and R^{b3b} are each independently C₂₋₁₀ alkyl.
A12. The compound of any one of embodiments 1-11, or a salt thereof, wherein Raba R^{a3b} R^{b3a}, and R^{b30} are each independently H or optionally substituted with a heterocyclyl comprising a disulfide bond.
A13. The compound of any one of embodiments 1-12, or a salt thereof, wherein R^{a3a}, R^{a3b} R^{b3a}, and R^{b3b} are each independently H or optionally substituted with 1,2-dithiolanyl.
A14. The compound of any one of embodiments 1-13, or a salt thereof, wherein none of Rasa R^{a3b}, R^{b3a}, and R^{b3b} is H.
A15. The compound of any one of embodiments 1-14, or a salt thereof, wherein at least one of R^{a3a}, R^{a3b}, R^{b3a}, and R^{b3b} is substituted with a heterocyclyl.
A16. The compound of any one of embodiments 1-8, or a salt thereof, wherein R^{a3} and R^{b3} are each independently

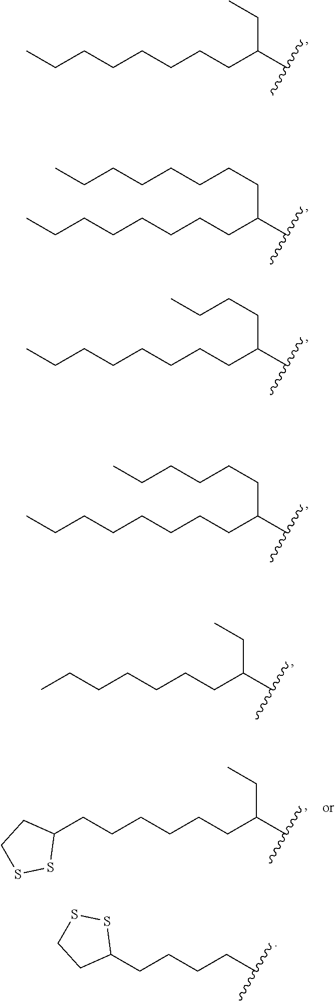

A17. The compound of any one of embodiments 1-16, or a salt thereof, wherein R^{a3} and R^{b3} are the same.
A18. The compound of any one of embodiments 1-17, or a salt thereof, wherein R^{c1} is —(CH₂)₂—.
A19. The compound of any one of embodiments 1-17, or a salt thereof, wherein R^{c1} is —(CH₂)₃—.
A20. The compound of any one of embodiments 1-17, or a salt thereof, wherein R^{c1} is —(CH₂)₄—.
A21. The compound of any one of embodiments 1-20, or a salt thereof, wherein R^{c2} is methyl.
A22. The compound of any one of embodiments 1-20, or a salt thereof, wherein R^{c2} is ethyl.
A23. The compound of any one of embodiments 1-22, or a salt thereof, wherein R^{c3} is C₁₋₆ alkyl.
A24. The compound of any one of embodiments 1-23, or a salt thereof, wherein R^{c3} is methyl.
A25. The compound of any one of embodiments 1-23, or a salt thereof, wherein R^{c3} is ethyl.
A26. The compound of any one of embodiments 1-17, or a salt thereof, wherein when R^{c1} is —(CH₂)₂— and R^{c2} is methyl, then R^{c3} is not methyl.
A27. The compound of any one of embodiments 1-22, or a salt thereof, wherein R^{c3} is

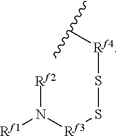

A28. The compound of embodiment 27, or a salt thereof, wherein R^{f1} is H.
A29. The compound of embodiment 27, or a salt thereof, wherein R^{f1} is

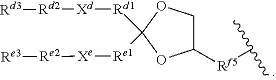

A30. The compound of any one of embodiments 27-29, or a salt thereof, wherein R^{f2} is methyl.
A31. The compound of any one of embodiments 27-29, or a salt thereof, wherein R^{f2} is ethyl.
A32. The compound of any one of embodiments 27-31, or a salt thereof, wherein R^{f3} and R^{f4} are each —(CH₂)₂—.
A33. The compound of any one of embodiments 29-32, or a salt thereof, wherein R^{f5} is —(CH₂)₂—.
A34. The compound of any one of embodiments 29-32, or a salt thereof, wherein R^{f5} is —(CH₂)₃—.
A35. The compound of any one of embodiments 29-32, or a salt thereof, wherein R^{f5} is —(CH₂)₄—.
A36. The compound of any one of embodiments 29-35, or a salt thereof, wherein R^{d1} and R^{e1} are each independently a linear C₁₋₁₂ alkyelene.
A37. The compound of any one of embodiments 29-36, or a salt thereof, wherein R^{d1} and R^{e1} are each independently C₅₋₁₀ alkylene.
A38. The compound of embodiment 37, or a salt thereof, wherein R^{d1} and R^{e1} are each —(CH₂)₇—.
A39. The compound of any one of embodiments 29-38, or a salt thereof, wherein X^{d} and X^{e} are each —C(O)O—*.
A40. The compound of any one of embodiments 29-38, or a salt thereof, wherein X^{d} and X^{e} are each —OC(O)—*.
A41. The compound of any one of embodiments 29-40, or a salt thereof, wherein R^{d2} and R^{e2} are each a bond.
A42. The compound of any one of embodiments 29-40, or a salt thereof, wherein R^{d2} and R^{e2} are each —CH₂—.
A43. The compound of any one of embodiments 29-42, or a salt thereof, wherein no more than one of R^{d3a}, R^{d3b}, R^{e3a}, and R^{e3b} is H.
A44. The compound of any one of embodiments 29-43, or a salt thereof, wherein R^{d3a}, R^{d3b} R^{e3a}, and R^{e3b} are each independently a linear C₁₋₁₂ alkyl.
A45. The compound of any one of embodiments 29-44, or a salt thereof, wherein R^{d3a}, R^{d3b} R^{e3a}, and R^{e3b} are each independently C₂₋₁₀ alkyl.
A46. The compound of any one of embodiments 29-45, or a salt thereof, wherein R^{d3}a, R^{d3b} R^{e3a}, and R^{e3b} are each independently H or optionally substituted with a heterocyclyl comprising a disulfide bond.
A47. The compound of any one of embodiments 29-46, or a salt thereof, wherein R^{d3}a, R^{d3b}, R^{e3a}, and R^{e3b} are each independently H or optionally substituted with 1,2-dithiolanyl.
A48. The compound of any one of embodiments 29-47, or a salt thereof, wherein none of R^{d3a} R^{d3b}, R^{e3a}, and R^{e3b} is H.
A49. The compound of any one of embodiments 29-48, or a salt thereof, wherein at least one of R^{d3a}, R^{d3b}, R^{c3a}, and R^{c3b} is substituted with a heterocyclyl.
A50. The compound of any one of embodiments 29-42, or a salt thereof, wherein R^{d3} and R^{e3} are each independently

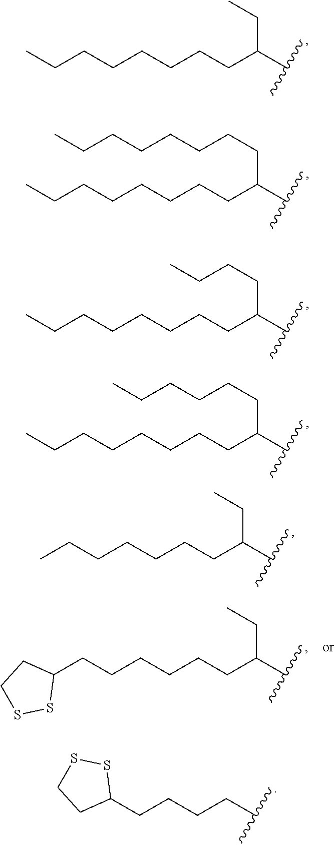

A51. The compound of any one of embodiments 29-50, or a salt thereof, wherein R^{d3} and R^{e3} are the same.
A52. The compound of embodiment 1, or a salt thereof, wherein the compound or the salt thereof is selected from the group consisting of the compounds of Table 1 and salts thereof.
A53. A lipid nanoparticle (LNP) comprising a lipid blend for targeted delivery of a nucleic acid into an immune cell, the lipid blend comprising a lipid-immune cell targeting group conjugate comprising the compound of Formula (II): [Lipid]-[optional linker]-[immune cell targeting group].
A54. The LNP of embodiment 53, further comprising an ionizable cationic lipid.
A55. The LNP of embodiment 54, wherein the ionizable cationic lipid comprises the compound of any one of embodiments 1 to 52, or a salt thereof.
A56. The LNP of any one of embodiments 53 to 55, further comprising a nucleic acid, wherein the nucleic acid is encapsulated in the LNP.
A57. The LNP of any one of embodiments 53 to 56, wherein the immune cell targeting group comprises an antibody that binds a T cell antigen.
A58. The LNP of embodiment 57, wherein the T cell antigen is CD3, CD4, CD7, CD8, or a combination thereof (e.g., both CD3 and CD8, both CD4 and CD8, or both CD7 and CD8).
A59. The LNP of any one of embodiments 53 to 58, wherein the immune cell targeting group comprises an antibody that binds a Natural Killer (NK) cell antigen.
A60. The LNP of embodiment 59, wherein the NK cell antigen is CD7, CD8, CD56, or a combination thereof (e.g., both CD7 and CD8).
A61. The LNP of any one of embodiments 53 to 60, wherein the immune cell targeting group comprises an antibody that binds a macrophage antigen, a monocyte antigen, and/or a dendritic antigen.
A62. The LNP of embodiment 61, wherein the macrophage comprises an M1 macrophage, an M2 macrophage, or both.
A63. The LNP of embodiment 61 or 62, wherein the macrophage comprises an M2a macrophage, an M2b macrophage, an M2c macrophage, or any combination thereof.
A64. The LNP of embodiment 61, wherein the macrophage antigen comprises CDIIB, CD68, CD80, CD86, TRL-2, TRL-4, INOS, MHC-II, CD163, CD206, CD209, FIZZ1, or Ym1/2, or any combination thereof.
A65. The LNP of embodiment 64, wherein the macrophage antigen comprises CD206.
A66. The LNP of any one of embodiments 53 to 65, wherein the immune cell targeting group is covalently coupled to a lipid in the lipid blend via a polyethylene glycol (PEG) containing linker.
A67. The LNP of embodiment 66, wherein the lipid covalently coupled to the immune cell targeting group via a PEG containing linker is distearoylglycerol (DSG), distearoyl-phosphatidylethanolamine (DSPE), dimyrstoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.
A68. The LNP of embodiment 66 or 67, wherein the PEG is PEG 2000 or PEG 3400.
A69. The LNP of any one of embodiments 53 to 68, wherein the lipid-immune cell targeting group conjugate is present in the lipid blend in a range of 0.001 to 0.5 mole percent (e.g., 0.002-0.2 mole percent).
A70. The LNP of any one of embodiments 53 to 69, wherein the lipid blend further comprises one or more of a structural lipid, a neutral phospholipid, and a free PEG-lipid.
A71. The LNP of any one of embodiments 53 to 70, wherein the ionizable cationic lipid is present in the lipid blend in a range of 30-70 (e.g., 40-60) mole percent.
A72. The LNP of embodiment 70 or 71, wherein the structural lipid is sterol.
A73. The LNP of embodiment 72, wherein the sterol is present in the lipid blend in a range of 20-70 (e.g., 30-50) mole percent.
A74. The LNP of embodiment 72 or 73, wherein the sterol is cholesterol.
A75. The LNP of any one of embodiments 70 to 74, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin.
A76. The LNP of any one of embodiments 70 to 75, wherein the neutral phospholipid is present in the lipid blend in a range of 5-15 mole percent.
A77. The LNP of any one of embodiments 70 to 76, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.
A78. The LNP of any one of embodiments 70 to 77, wherein the free PEG-lipid comprises a diacylphosphatidylethanolamine comprising dimyristoyl (C14) chain, Dipalmitoyl (C16) chain or Distearoyl (C18) chain, and optionally the free PEG-lipid comprises PEG-DPG and PEG-DMG.
A79. The LNP of any one of embodiments 70 to 78, wherein the free PEG-lipid is present in the lipid blend in a range of about 1 to about 4 mole percent, such as about 0.5 to about 2.5 mole percent.
A80. The LNP of any one of embodiments 70 to 79, wherein the free PEG-lipid comprises the same or a different lipid as the lipid in the lipid-immune cell targeting group conjugate.
A81. The LNP of any one of embodiments 53 to 80, wherein the LNP has a mean diameter in the range of 50-200 nm.
A82. The LNP of embodiment 81, wherein the LNP has a mean diameter of between about 75 nm and about 80 nm.
A83. The LNP of any one of embodiments 53 to 82, wherein the LNP has a polydispersity index in a range from about 0.01 to about 0.5.
A84. The LNP of any one of embodiments 53 to 83, wherein the LNP has a pKa of between about 5.0 and about 8.0.
A85. The LNP of any one of embodiments 53 to 84, wherein the nucleic acid is DNA or RNA.
A86. The LNP of embodiment 85, wherein the RNA is an mRNA.
A87. The LNP of embodiment 86, wherein the mRNA encodes a receptor, a growth factor, a hormone, a cytokine, an antibody, an antigen, an enzyme, or a vaccine.
A88. The LNP of embodiment 86, wherein the mRNA encodes a polypeptide capable of regulating immune response in the immune cell.
A89. The LNP of embodiment 86, wherein the mRNA encodes a polypeptide capable of reprogramming the immune cell.
A90. The LNP of embodiment 86, wherein the mRNA encodes a synthetic T cell receptor (synTCR) or a Chimeric Antigen Receptor (CAR).
A91. The LNP of embodiment 86, wherein the mRNA encodes polypeptide capable of reprogramming an M2 macrophage to an M1 macrophage.
A92. The LNP of embodiment 86, wherein the RNA is tRNA, siRNA, gRNA, or microRNA.
A93. The LNP of any one of embodiments 53 to 92, wherein the immune cell targeting group comprises an antibody, and the antibody is a Fab or an immunoglobulin single variable (ISV) domain (e.g., a Nanobody)
A94. The LNP of embodiment 93, wherein the antibody is a human or humanized antibody.
A95. The LNP of any one of embodiments 53 to 94, wherein the immune cell targeting group comprises a Fab, F(ab′)₂, Fab′-SH, Fv, or scFv fragment, or any combination thereof.
A96. The LNP of any one of embodiments 53 to 95, wherein the immune cell targeting group comprises a Fab.
A97. The LNP of embodiment 96, wherein the Fab is engineered to knock out the natural interchain disulfide bond at the C-terminus.
A98. The LNP of embodiment 97, wherein the Fab comprises a heavy chain fragment that comprises C233S substitution, and a light chain fragment that comprises C214S substitution, numbering according to Kabat.
A99. The LNP of embodiment 96, wherein the Fab has a non-natural interchain disulfide bond (e.g., an engineered, buried interchain disulfide bond).
A100. The LNP of embodiment 99, wherein the Fab comprises F174C substitution in the heavy chain fragment, and S176C substitution in the light chain fragment, numbering according to Kabat.
A101. The LNP of embodiment 96, wherein the Fab comprises a cysteine at the C-terminus of the heavy or light chain fragment.
A102. The LNP of embodiment 101, wherein the Fab further comprises one or more amino acids between the heavy chain fragment of the Fab and the C-terminal cysteine.
A103. The LNP of embodiment 96, wherein the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the immune cell targeting group further comprises a single chain variable fragment (scFv) linked to the C-terminus of the light chain constant domain by an amino acid linker.
A104. The LNP of embodiment 93 or 94, wherein the immune cell targeting group comprises an ISV domain.
A105. The LNP of embodiment 104, wherein the ISV domain is Nanobody® ISV.
A106. The LNP of embodiment 104 or 105, wherein the ISV domain comprises a cysteine at the C-terminus.
A107. The LNP of embodiment 106, wherein the ISV domain comprises a VIII domain and further comprises a spacer comprising one or more amino acids between the V_{HH} domain and the C-terminal cysteine.
A108. The LNP of any one of embodiments 104 to 107, wherein the immune cell targeting group comprises two or more V_{HH} domains.
A109. The LNP of embodiment 108, wherein the two or more Van domains are linked by an amino acid linker.
A110. The LNP of embodiment 108, wherein the immune cell targeting group comprises a first V_{HH} domain linked to an antibody CH1 domain and a second V_{HH} domain linked to an antibody light chain constant domain.

A111. The LNP of embodiment 110, wherein the antibody CH₁ domain and the antibody light chain constant domain are linked by one or more disulfide bonds.
A112. The LNP of any one of embodiments 104 to 108, wherein the immune cell targeting group comprises a V_{HH} domain linked to an antibody CH1 domain, and wherein the antibody CH1 domain is linked to an antibody light chain constant domain by one or more disulfide bonds.
A113. The LNP of embodiment 111 or 112, wherein the CH1 domain comprises F174C and C233S substitutions, and the light chain constant domain comprises S176C and C214S substitutions, numbering according to Kabat.
A114. The LNP of any one of embodiments 53 to 113, wherein the immune cell targeting group comprises a Fab that comprises:
   (a) a heavy chain fragment comprising the amino acid sequence of SEQ ID NO: 1 and a light chain fragment comprising the amino acid sequence of SEQ ID NO: 2 or 3; or
   (b) a heavy chain fragment comprising the amino acid sequence of SEQ ID NO: 6 and a light chain fragment comprising the amino acid sequence of SEQ ID NO: 7.
A115. The LNP of any one of embodiments 53 to 114, wherein the LNP is for delivering a nucleic acid into an immune cell, and wherein the immune cell is an NK cell, and the immune cell targeting group comprises an antibody that binds CD56.
A116. The LNP of any one of embodiments 70 to 114, wherein the LNP is for delivering a nucleic acid into an immune cell, and wherein the immune cell targeting group comprises an antibody that binds CD7 or CD8, and the free PEG-lipid is DMG-PEG or PEG-DPG.
A117. The LNP of any one of embodiments 53 to 114, wherein the LNP is for delivering a nucleic acid into an immune cell, and wherein the immune cell is a macrophage, and the immune cell targeting group comprises an antibody that binds CD206.
A118. The LNP of any one of embodiments 70 to 114, wherein the LNP is for delivering a nucleic acid into an immune cell, and wherein the immune cell targeting group comprises an antibody that binds CD206, and the free PEG-lipid is DMG-PEG or PEG-DPG.
A119. The LNP of any one of embodiments 70 to 118, wherein the free PEG-lipid comprises a PEG having a molecular weight of at least 2000 daltons
A120. The LNP of embodiment 119, wherein the PEG has a molecular weight of about 3000 to 5000 daltons.
A121. The LNP of any one of embodiments 93 to 120, wherein the antibody is a Fab.
A122. The LNP of embodiment 121, wherein the Fab binds CD3, and the free PEG-lipid in the LNP comprises a PEG having a molecular weight of about 2000 daltons.
A123. The LNP of embodiment 121, wherein the Fab is an anti-CD4 antibody, and the free PEG-lipid in the LNP comprises a PEG having a molecular weight of about 3000 to 3500 daltons.
A124. The LNP of embodiment 121, wherein the Fab binds CD206, and the free PEG-lipid in the LNP comprises a PEG having a molecular weight of about 2000 daltons.
A125. The LNP of embodiment 121, wherein the Fab is an anti-CD206 antibody, and the free PEG-lipid in the LNP comprises a PEG having a molecular weight of about 3000 to 3500 daltons.
A126. The LNP of any one of embodiments 53 to 114, wherein the LNP is for delivering a nucleic acid into an immune cell, and wherein the LNP binds CD3, and also binds CD11a or CD18 of the immune cell.
A127. The LNP of embodiment 126, wherein the LNP comprises two conjugates, wherein the first conjugate comprises an antibody that binds CD3, and the second conjugate comprises an antibody that binds CD11a or CD18.
A128. The LNP of embodiment 126, wherein the LNP comprises one conjugate, and the conjugate comprises a bispecific antibody that binds both CD3 and CD11a.
A129. The LNP of embodiment 126, wherein the LNP comprises one conjugate, and the conjugate comprises a bispecific antibody that binds both CD3 and CD18.
A130. The LNP of embodiment 128 or 129, wherein the bispecific antibody is an immunoglobulin single variable domain or Fab-ScFv.
A131. The LNP of any one of embodiments 53 to 114, wherein the LNP is for delivering a nucleic acid into an immune cell, and wherein the LNP binds CD7 and CD8 of the immune cell.
A132. The LNP of embodiment 131, wherein the LNP comprises two conjugates, wherein the first conjugate comprises an antibody that binds CD7, and a second conjugate that binds CD8.
A133. The LNP of embodiment 131, wherein the LNP comprises one conjugate, wherein the conjugate comprises a bispecific antibody that binds CD7 and CD8.
A134. The LNP of embodiment 133, wherein the bispecific antibody is an immunoglobulin single variable domain or Fab-ScFv.
A135. The LNP of any one of embodiments 53 to 114, wherein the LNP is for delivering a nucleic acid into an immune cell, and wherein the LNP binds a first macrophage antigen, and also binds a second macrophage antigen.
A136. The LNP of embodiment 135, wherein the LNP comprises two conjugates, wherein the first conjugate comprises an antibody that binds the first macrophage antigen, and the second conjugate comprises an antibody that binds the second macrophage antigen.
A137. The LNP of embodiment 135, wherein the LNP comprises one conjugate, and the conjugate comprises a bispecific antibody that binds both the first macrophage antigen and the second macrophage antigen.
A138. The LNP of embodiment 137, wherein the bispecific antibody is an immunoglobulin single variable domain or Fab-ScFv.
A139. The LNP of any one of embodiments 53 to 114, wherein the LNP binds to a first antigen on the surface of the first type of immune cell, and also binds to a second antigen on the surface of the second type of immune cell.
A140. The LNP of embodiment 139, wherein the two different types of immune cells are CD4+ T cells and CD8+ T cell.
A141. The LNP of embodiment 139, wherein the first type of immune cell is a first macrophage, and the second type of immune cell is a second macrophage, a T-cell, or an NK cell.
A142. The LNP of embodiment 139, wherein the LNP comprises two conjugates, and the first conjugate comprises a first antibody that binds to the first antigen of the first type of immune cell, and the second conjugate comprises a second antibody that binds to the second antigen of the second type of immune cell.
A143. The LNP of embodiment 139, wherein the LNP comprises one conjugate, and the conjugate comprises a bispecific antibody, and the bispecific antibody binds to both the first antigen on the first type of immune cell, and the second antigen on the second type of immune cells.
A144. The LNP of any one of embodiments 53 to 114, wherein the bispecific antibody is an immunoglobulin single variable domain or a Fab-ScFv.
A145. The LNP of any one of embodiments 53 to 114, wherein the LNP is for delivering a nucleic acid into an immune cell, and wherein the immune cell targeting group comprises a single antibody that binds to CD3 or CD7.
A146. The LNP of any one of embodiments 53 to 114, wherein the LNP is for delivering a nucleic acid into an immune cell, and wherein the immune cell targeting group binds to CD7, CD8, or both CD7 and CD8.
A147. The LNP of any one of embodiments 53 to 114, wherein the LNP is for delivering a nucleic acid into both T cells and NK cells, wherein the immune cell targeting group binds to
   (a) both CD3 and CD56;
   (b) both CD8 and CD56; or
   (c) both CD7 and CD56.
A148. The LNP of any one of embodiments 53 to 114, wherein the LNP is for delivering a nucleic acid into an immune cell, and wherein the immune cell targeting group comprises a Fab lacking the native interchain disulfide bond.
A149. The LNP of embodiment 148, wherein the Fab is engineered to replace one or both cysteines on the native constant light chain and the native constant heavy chain that form the native interchain disulfide with a non-cysteine amino acid, therefor to remove the native interchain disulfide bond in the Fab.
A150. A method of targeting the delivery of a nucleic acid to an immune cell of a subject, the method comprising contacting the immune cell with the LNP of any one of embodiments 53 to 149, wherein the LNP comprises the nucleic acid.
A151. A method of expressing a polypeptide of interest in a targeted immune cell of a subject, the method comprising contacting the immune cell with the LNP of any one of embodiments 53 to 149, wherein the LNP comprises a nucleic acid encoding the polypeptide.
A152. A method of modulating cellular function of a target immune cell of a subject, the method comprising administering to the subject the LNP of any one of embodiments 53 to 149, wherein the LNP comprises a nucleic acid modulates the cellular function of the immune cell.
A153. A method of treating, ameliorating, or preventing a symptom of a disorder or disease in a subject, the method comprising administering to the subject an LNP for delivering a nucleic acid into an immune cell of the subject, wherein the LNP is any one of embodiments 53 to 149, wherein the LNP comprises the nucleic acid.
A154. The method of embodiment 153, wherein the disorder is an immune disorder, an inflammatory disorder, or cancer.
A155. The method of embodiment 153, wherein the nucleic acid encodes an antigen for use in a therapeutic or prophylactic vaccine for treating or preventing cancer or an infection by a pathogen.
A156. The method of any one of embodiments 150 to 155, wherein no more than 5% non-immune cells are transfected by the LNP.
A157. The method of any one of embodiments 150 to 156, wherein half-life of the nucleic acid delivered by the LNP or a polypeptide encoded by the nucleic acid delivered by the LNP is at least 10% longer than half-life of nucleic acid delivered by a reference LNP or a polypeptide encoded by the nucleic acid delivered by the reference LNP.
A158. The method of any one of embodiments 150 to 157, wherein at least 10% immune cells are transfected by the LNP.
A159. The method of any one of embodiments 150 to 158, wherein expression level of the nucleic acid delivered by the LNP is at least 10% higher than expression level of nucleic acid delivered by a reference LNP.
A160. The method of any one of embodiments 150 to 159, wherein the LNP comprises the compound of any one of embodiments 1 to 52, or a salt thereof.
A161. The method of embodiment 160, wherein (i) R^{c3} is

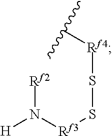

or (ii) R^{a3} and R^{b3} are each independently

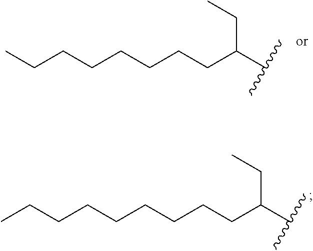 [SMILES: *C(CC)CCCCCCCC ; *C(CC)CCCCCCCCC]

or both (i) and (ii).
A162. The method of embodiment 160, wherein the LNP comprises Lipid 28, Lipid 6, Lipid 12, Lipid 1, or Lipid 7 of Table 1, or a salt of thereof.
A163. The method of embodiment 160, wherein (i) R^{a3} and R^{b3} are each independently

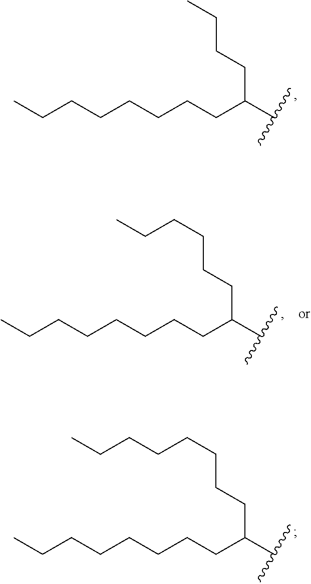 [SMILES: *C(CCCC)CCCCCCCC ; *C(CCCCCC)CCCCCCCC ; *C(CCCCCCCC)CCCCCCCC]

or at least one of R^{a3} and R^{b3} is

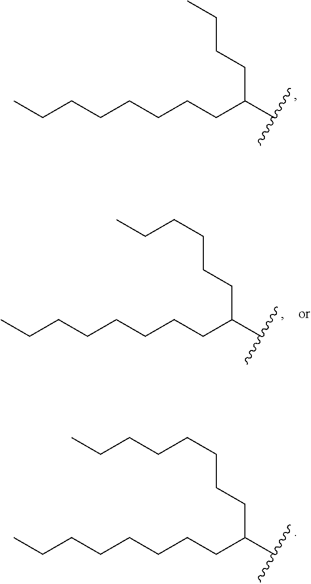 [SMILES: *C(CCCC)CCCCCCCC ; *C(CCCCCC)CCCCCCCC ; *C(CCCCCCCC)CCCCCCCC]

A164. The method of embodiment 160, wherein the LNP comprises Lipid 9 or Lipid 10, or a salt thereof.
A165. A method of targeting the delivery of a nucleic acid to a non-liver cell, the method comprising contacting the non-liver cell with an LNP comprising the compound of any one of embodiments 1-52, or a salt thereof, wherein (i) R^{c3} is

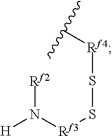

or (ii)
R^{a3} and R^{b3} are each independently

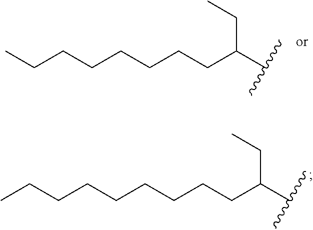 [SMILES: *C(CC)CCCCCCCC ; *C(CC)CCCCCCCCC]

or both (i) and (ii).
A166. The method of embodiment 165, wherein the LNP comprises Lipid 28, Lipid 6, Lipid 12, Lipid 1, or Lipid 7 of Table 1, or a salt of thereof.
A167. A method of targeting the delivery of a nucleic acid to a liver cell, the method comprising contacting the liver cell with an LNP comprising the compound of any one of embodiments 1-52, or a salt thereof, wherein (i) R^{a3} and R^{b3} are each independently

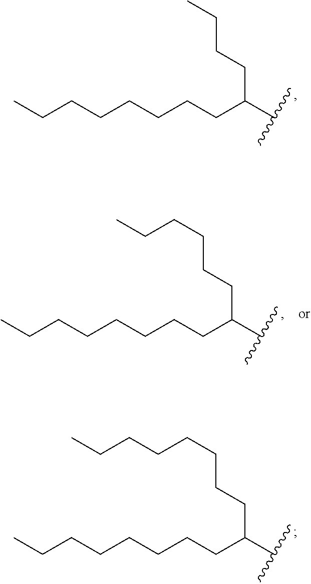 [SMILES: *C(CCCC)CCCCCCCC ; *C(CCCCCC)CCCCCCCC ; *C(CCCCCCCC)CCCCCCCC]

or at least one of R^{a3} and R^{b3} is

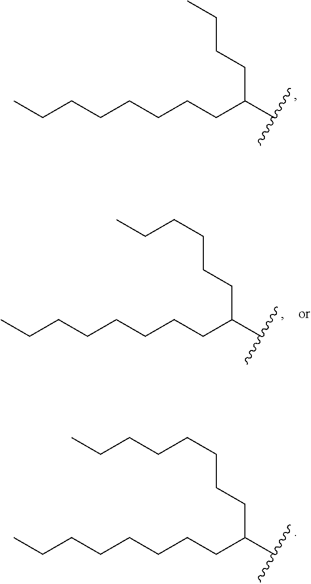 [SMILES: *C(CCCC)CCCCCCCC ; *C(CCCCCC)CCCCCCCC ; *C(CCCCCCCC)CCCCCCCC]

A168. The method of embodiment 167, wherein the LNP comprises Lipid 9 or Lipid 10 of Table 1, or a salt thereof.

### Enumerated Embodiments Set B

The embodiment numbers refenced in this set refers to those within set B. For example, “embodiment 1” recited in embodiment B2 refers to embodiment B1.
B1. A compound of Formula (I):

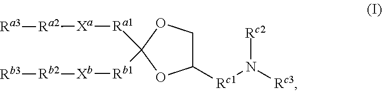

or a salt thereof, wherein:
   R^{a1} and R^{b1} are each independently C₁₋₁₂ alkylene;
   X^{a} and X″ are each independently —C(O)O—* or —OC(O)—*, wherein * indicates the point of attachment to R^{a1} or R^{b1};
   R^{a2} and R^{b2} are each independently a bond or C₁₋₃ alkylene;
   R^{a3} is

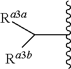

and R^{b3} is

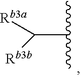

wherein R^{a3a}, R^{a3b}, R^{b3a}, and R^{b3} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3;
R^{c1} is C₁₋₆ alkylene;
R^{c2} is H or C16 alkyl; and
R^{c3} is C₁₋₆ alkyl,

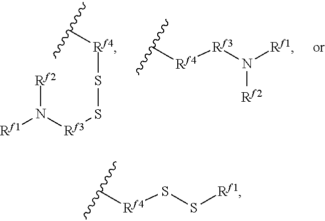

wherein:
R^{f1} is H, C₁₋₆ alkyl, or

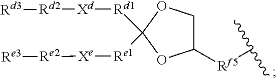

R^{f2} is H, C₁₋₆ alkyl, or —C(O)O—C₂₋₆ alkenyl;
R^{f3}, R^{f4}, and R^{f5} are each independently C₁₋₆ alkylene; and
R^{d1} and R^{e1} are each independently C₁₋₁₂ alkylene;
X^{d} and X^{e} are each independently —C(O)O—* or —OC(O)—*, wherein * indicates the point of attachment to R^{d1} or Rel;
R^{d2} and R^{e2} are each independently a bond or C₁₋₃ alkylene; and
R^{d3} is

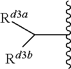

and R^{c3} is

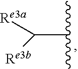

wherein R^{d3a}, R^{d3b}, R^{e3a}, and R^{e3b} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3;
with the proviso that when R^{c1} is —CH₂— and R^{c2} and R^{c3} are each methyl, then none of R^{a3b}, R^{a3b}, R^{b3a}, and R^{b3b} is H; when Rol is —(CH₂)₂— and R^{c2} and R^{c3} are each methyl, then R^{b3a} is not H and R^{b3b} is ethyl; when R^{c1} is —(CH₂)₂— and R^{c2}, R^{c3}, or both R^{c2} and R^{c3} are not methyl, then none of R^{a32}, R^{a3b}, R^{b3a}, and R^{b3b} is H; when R^{c1} is —(CH₂)₃—, R^{c2} and R^{c3} are each methyl, and one of R^{a3a}, R^{a3b}, R^{b3b}, and R^{b3b} is H, then at least one of R^{a3a}, R^{a3b}, R^{b3a}, and R^{b3b} that is not His substituted with a heterocylyl; and when R^{c1} is —(CH₂)₄—, R^{c2} and R^{o3} are each methyl, then none of R^{a3a}, R^{a3b}, R^{b3a}, and R^{b3b} is H.
B2. The compound of embodiment 1, or a salt thereof, wherein R^{a1} and R^{b1} are each independently a linear C₁₋₁₂ alkylene.
B3. The compound of embodiment 1 or 2, or a salt thereof, wherein R^{a1} and R^{b1} are each independently C₅₋₁₀ alkylene.
B4. The compound of embodiment 1, or a salt thereof, wherein R^{a1} and R^{b1} are each —(CH₂)₇—,
B5. The compound of any one of embodiments 1-4, or a salt thereof, wherein X^{a} and X^{b} are each —C(O)O—*.
B6. The compound of any one of embodiments 1-4, or a salt thereof, wherein X^{a} and X^{b} are each —OC(O)—*.
B7. The compound of any one of embodiments 1-6, or a salt thereof, wherein R^{a2} and R^{b2} are each a bond.
B8. The compound of any one of embodiments 1-6, or a salt thereof, wherein R^{a2} and R^{b2} are each —CH₂—.
B9. The compound of any one of embodiments 1-8, or a salt thereof, wherein no more than one of R^{a3a}, R^{a3b}, R^{b3a}, and R^{b3b} is H.
B10. The compound of any one of embodiments 1-9, or a salt thereof, wherein R^{a3a}, R^{a3b}, R^{b3a} and R^{b3b} are each independently a linear C₁₋₁₂ alkyl.
B11. The compound of any one of embodiments 1-10, or a salt thereof, wherein Raba R^{a3b} R^{b3a}, and R^{b3b} are each independently C₂₋₁₀ alkyl.
B12. The compound of any one of embodiments 1-11, or a salt thereof, wherein none of R^{a3a} R^{a3b}, R^{b3a}, and R^{b3b} is H.
B13. The compound of any one of embodiments 1-12, or a salt thereof, wherein at least one of R^{a3a}, R^{a3b}, R^{b3a}, and R^{b3b} is C₁₋₁₂ alkyl substituted with a 5- to 10-membered heterocyclyl comprising a disulfide bond or —(C₁₋₁₀ alkylene)-Sₙ—(C₁₋₁₀ alkyl).
B14. The compound of embodiment 13, or a salt thereof, wherein the heterocyclyl comprising a disulfide bond is 1,2-dithiolanyl.
B15. The compound of any one of embodiments 1-8, or a salt thereof, wherein R^{a3} and R^{b3} are each independently

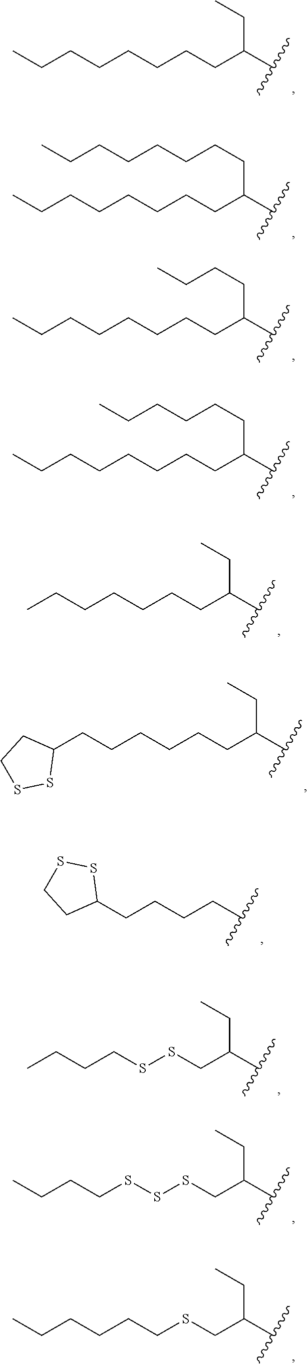

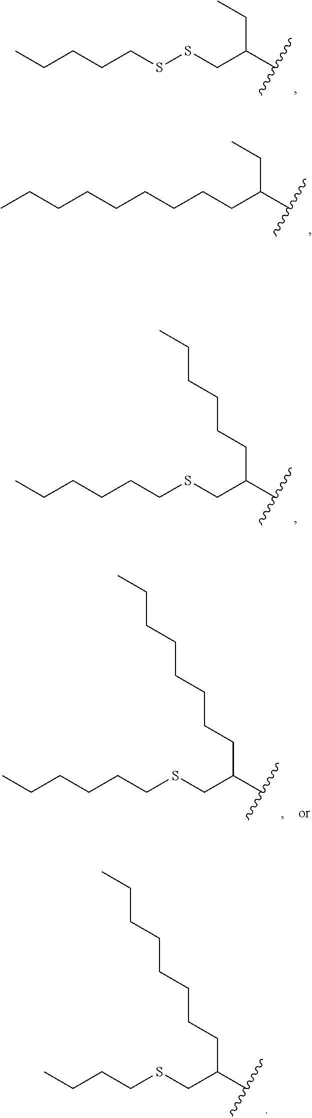 [SMILES: *C(CC)CSSCCCCC ; *C(CC)CCCCCCCCC ; *C(CCCCCC)CSCCCCCC ; *C(CCCCCCCC)CSCCCCCC ; *C(CCCCCCCC)CSCCCC]

B16. The compound of any one of embodiments 1-15, or a salt thereof, wherein R^{a3} and R^{b3} are the same.
B17. The compound of any one of embodiments 1-16, or a salt thereof, wherein R^{c1} is —(CH₂)₂—.
B18. The compound of any one of embodiments 1-16, or a salt thereof, wherein R^{c1} is —(CH₂)₃—.
B19. The compound of any one of embodiments 1-16, or a salt thereof, wherein R^{c1} is —(CH₂)₄—.
B20. The compound of any one of embodiments 1-19, or a salt thereof, wherein R^{c2} is methyl.
B21. The compound of any one of embodiments 1-19, or a salt thereof, wherein R^{c2} is ethyl.
B22. The compound of any one of embodiments 1-21, or a salt thereof, wherein R^{c3} is C₁₋₆ alkyl.
B23. The compound of any one of embodiments 1-21, or a salt thereof, wherein R^{c3} is methyl.
B24. The compound of any one of embodiments 1-21, or a salt thereof, wherein R^{c3} is ethyl.
B25. The compound of any one of embodiments 1-16, or a salt thereof, wherein when R^{c1} is —(CH₂)₂— and R^{c2} is methyl, then R^{c3} is not methyl.
B26. The compound of any one of embodiments 1-21, or a salt thereof, wherein R^{c3} is

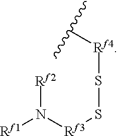

B27. The compound of any one of embodiments 1-21, or a salt thereof, wherein R^{c3} is

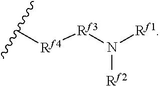

B28. The compound of any one of embodiments 1-21, or a salt thereof, wherein R^{c3} is

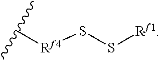

B29. The compound of any one of embodiments 26-28, or a salt thereof, wherein R^{f1} is H.
B30. The compound of any one of embodiments 26-28, or a salt thereof, wherein R^{f1} is C₁₋₆ alkyl.
B31. The compound of any one of embodiments 26-28, or a salt thereof, wherein R^{f1} is methyl.
B32. The compound of any one of embodiments 26-28, or a salt thereof, wherein R^{f1} is n-butyl.
B33. The compound of any one of embodiments 26-28, or a salt thereof, wherein Rf is

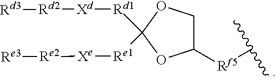

B34. The compound of any one of embodiments 26, 27, and 29-33, or a salt thereof, wherein R^{f2} is H.
B35. The compound of any one of embodiments 26, 27, and 29-33, or a salt thereof, wherein R^{f2} is C₁₋₆ alkyl.
B36. The compound of any one of embodiments 26, 27, and 29-33, or a salt thereof, wherein R² is methyl.
B37. The compound of any one of embodiments 26, 27, and 29-33, or a salt thereof, wherein R^{f2} is ethyl.
B38. The compound of any one of embodiments 26, 27, and 29-33, or a salt thereof, wherein R^{f2} is —C(O)O—C₂₋₆ alkenyl.
B39. The compound of embodiment 38, or a slat thereof, wherein R^{f2} is —C(O)O—CH₂CH═CH₂.
B40. The compound of any one of embodiments 26, 27, and 29-39, or a salt thereof, wherein R^{f3} and R^{f4} are each —(CH₂)₂—.
B41. The compound of any one of embodiments 26, 27, and 29-39, or a salt thereof, wherein R^{f3} and R^{f4} are each —(CH₂)₃—.
B42. The compound of any one of embodiments 26-39, or a salt thereof, wherein R^{f4} is —(CH₂)₂—.
B43. The compound of any one of embodiments 33-42, or a salt thereof, wherein R^{f5} is —(CH₂)₂—.
B44. The compound of any one of embodiments 33-42, or a salt thereof, wherein R^{f5} is —(CH₂)₃—.
B45. The compound of any one of embodiments 33-42, or a salt thereof, wherein R^{f5} is —(CH₂)₄—.
B46. The compound of any one of embodiments 33-45, or a salt thereof, wherein R^{d1} and Rel are each independently a linear C₁₋₁₂ alkyelene.
B47. The compound of any one of embodiments 33-46, or a salt thereof, wherein R^{d1} and Rel are each independently C5-10 alkylene.
B48. The compound of embodiment 47, or a salt thereof, wherein R^{d1} and R^{e1} are each —(CH₂)₇—.
B49. The compound of any one of embodiments 33-48, or a salt thereof, wherein X^{d} and X^{e} are each —C(O)O—*.
B50. The compound of any one of embodiments 33-48, or a salt thereof, wherein X^{d} and X^{e} are each —OC(O)—*.
B51. The compound of any one of embodiments 33-50, or a salt thereof, wherein R^{d2} and R^{e2} are each a bond.
B52. The compound of any one of embodiments 33-50, or a salt thereof, wherein R^{d2} and R^{e2} are each —CH₂—.
B53. The compound of any one of embodiments 33-52, or a salt thereof, wherein no more than one of R^{d3a}, R^{d3b}, R^{c3a}, and R^{c3b} is H.
B54. The compound of any one of embodiments 33-53, or a salt thereof, wherein R^{d3a}, R^{d3b} R^{c3a}, and R^{c3b} are each independently a linear C₁₋₁₂ alkyl.
B55. The compound of any one of embodiments 33-54, or a salt thereof, wherein R^{d3a}, R^{d3b} R^{c3a}, and R^{c3b} are each independently C₂₋₁₀ alkyl.
B56. The compound of any one of embodiments 33-55, or a salt thereof, wherein none of R^{d3a} R^{d3b}, R^{c3a}, and R^{c3b} is H.
B57. The compound of any one of embodiments 33-56, or a salt thereof, wherein at least one of R^{d3}a, R^{d3b}, R^{e3a}, and R^{e3b} is C₁₋₁₂ alkyl substituted with a 5- to 10-membered heterocyclyl comprising a disulfide bond or —(C₁₋₁₀ alkylene)-Sₙ—(C₁₋₁₀ alkyl).
B58. The compound of embodiment 57, or a salt thereof, wherein the heterocyclyl comprising a disulfide bond is 1,2-dithiolanyl.
B59. The compound of any one of embodiments 33-52, or a salt thereof, wherein R^{d3} and R^{e3} are each independently

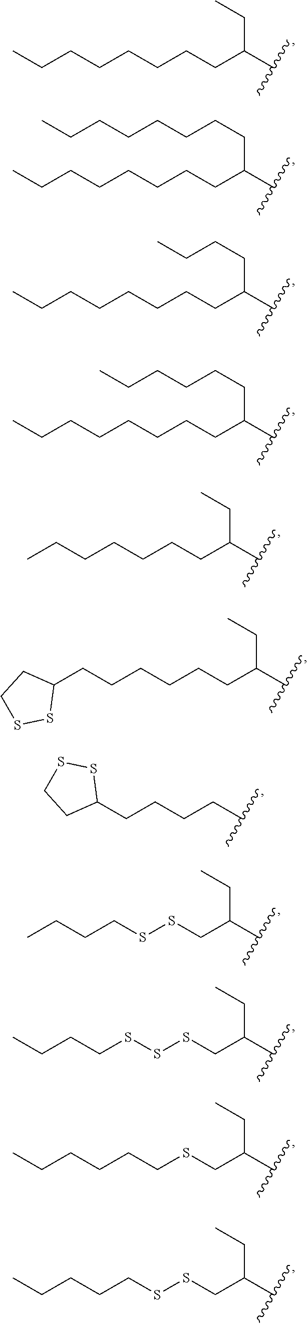

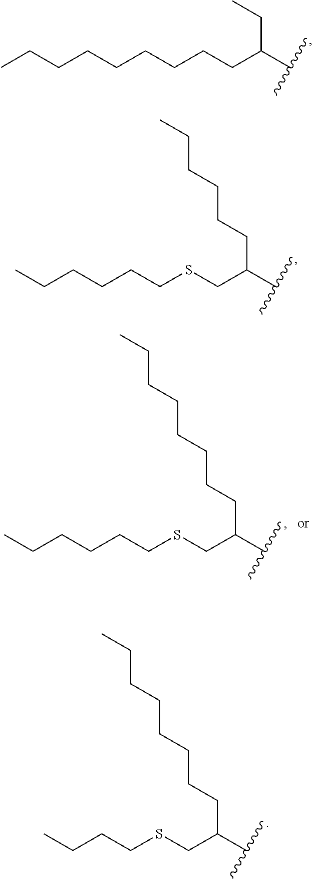 [SMILES: *C(CC)CCCCCCCCC ; *C(CCCCCC)CSCCCCCC ; *C(CCCCCCCC)CSCCCCCC ; *C(CCCCCCCC)CSCCCC]

B60. The compound of any one of embodiments 33-59, or a salt thereof, wherein R^{d3} and R^{e3} are the same.
B61. The compound of embodiment 1, or a salt thereof, wherein the compound or the salt thereof is selected from the group consisting of the compounds of Table 1 and salts thereof.
B62. A lipid nanoparticle (LNP) comprising a lipid blend for targeted delivery of a nucleic acid into an immune cell, the lipid blend comprising a lipid-immune cell targeting group conjugate comprising the compound of Formula (II): [Lipid]-[optional linker]-[immune cell targeting group].
B63. The LNP of embodiment 62, the lipid blend further comprising an ionizable cationic lipid.
B64. The LNP of embodiment 63, wherein the ionizable cationic lipid comprises the compound of any one of embodiments 1 to 61, or a salt thereof.
B65. The LNP of any one of embodiments 62 to 64, further comprising a nucleic acid, wherein the nucleic acid is encapsulated in the LNP.
B66. The LNP of any one of embodiments 62 to 65, wherein the immune cell targeting group comprises an antibody that binds a T cell antigen.
B67. The LNP of embodiment 66, wherein the T cell antigen is CD3, CD4, CD7, CD8, or a combination thereof (e.g., both CD3 and CD8, both CD4 and CD8, or both CD7 and CD8).
B68. The LNP of any one of embodiments 62 to 67, wherein the immune cell targeting group comprises an antibody that binds a Natural Killer (NK) cell antigen.
B69. The LNP of embodiment 68, wherein the NK cell antigen is CD7, CD8, CD56, or a combination thereof (e.g., both CD7 and CD8).
B70. The LNP of any one of embodiments 62 to 69, wherein the immune cell targeting group comprises an antibody that binds a macrophage antigen, a monocyte antigen, and/or a dendritic antigen.
B71. The LNP of embodiment 70, wherein the macrophage comprises an M1 macrophage, an M2 macrophage, or both.
B72. The LNP of embodiment 70 or 71, wherein the macrophage comprises an M2a macrophage, an M2b macrophage, an M2c macrophage, or any combination thereof.
B73. The LNP of embodiment 70, wherein the macrophage antigen comprises CDIIB, CD68, CD80, CD86, TRL-2, TRL-4, iNOS, MHC-II, CD163, CD206, CD209, FIZZ1, or Ym1/2, or any combination thereof.
B74. The LNP of embodiment 73, wherein the macrophage antigen comprises CD206.
B75. The LNP of any one of embodiments 62 to 74, wherein the immune cell targeting group is covalently coupled to a lipid in the lipid blend via a polyethylene glycol (PEG) containing linker.
B76. The LNP of embodiment 75, wherein the lipid covalently coupled to the immune cell targeting group via a PEG containing linker is distearoylglycerol (DSG), distearoyl-phosphatidylethanolamine (DSPE), dimyristoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.
B77. The LNP of embodiment 75 or 76, wherein the PEG is PEG 2000 or PEG 3400.
B78. The LNP of embodiment 77, wherein the PEG is PEG 3400.
B79. The LNP of any one of embodiments 62 to 78, wherein the lipid covalently coupled to the immune cell targeting group via a PEG containing linker is distearoyl-phosphatidylethanolamine (DSPE).
B80. The LNP of any one of embodiments 62 to 79, wherein the lipid-immune cell targeting group conjugate is present in the lipid blend in a range of 0.001 to 0.5 mole percent (e.g., 0.002-0.2 mole percent).
B81. The LNP of any one of embodiments 62 to 80, wherein the lipid blend further comprises one or more of a structural lipid, a neutral phospholipid, and a free PEG-lipid.
B82. The LNP of any one of embodiments 62 to 81, wherein the ionizable cationic lipid is present in the lipid blend in a range of 30-70 (e.g., 40-60) mol %.
B83. The LNP of embodiment 82, wherein the ionizable cationic lipid is present in the lipid blend in a range of about 48 mol % to about 50 mol %.
B84. The LNP of any one of embodiments 81 to 83, wherein the structural lipid is sterol.
B85. The LNP of embodiment 84, wherein the sterol is present in the lipid blend in a range of 20-70 (e.g., 30-50) mol %.
B86. The LNP of embodiment 85, wherein the sterol is present in the lipid blend in a range of about 27 mol % to about 29 mol %.
B87. The LNP of any one of embodiments 84 to 86, wherein the sterol is cholesterol, fecosterol, β-sitosterol, ergosterol, campesterol, stigmasterol, stigmastanol, or brassicasterol.
B88. The LNP of embodiment 87, wherein the sterol is cholesterol.
B89. The LNP of any one of embodiments 81 to 88, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin.
B90. The LNP of any one of embodiments 81 to 88, wherein the neutral phospholipid is 1,2-distearoyl-sn-glycero-3-phosphocholine (DSPC).
B91. The LNP of any one of embodiments 81 to 90, wherein the neutral phospholipid is present in the lipid blend in a range of 5-15 mol %.
B92. The LNP of any one of embodiments 81 to 90, wherein the neutral phospholipid is present in the lipid blend in a range of about 16 mol % to about 40 mol %.
B93. The LNP of any one of embodiments 81 to 90, wherein the neutral phospholipid is present in the lipid blend in a range of about 16 mol % to about 30 mol %.
B94. The LNP of any one of embodiments 81 to 90, wherein the neutral phospholipid is present in the lipid blend in a range of about 16 mol % to about 25 mol %.
B95. The LNP of any one of embodiments 81 to 90, wherein the neutral phospholipid is present in the lipid blend in a range of about 19 mol % to about 21 mol %.
B96. The LNP of any one of embodiments 81 to 90, wherein the neutral phospholipid is present in the lipid blend in about 20 mol %.
B97. The LNP of any one of embodiments 81 to 96, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, for example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyristoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.
B98. The LNP of any one of embodiments 81 to 96, wherein the free PEG-lipid is DPG-PEG2K
B99. The LNP of any one of embodiments 81 to 96, wherein the free PEG-lipid comprises a diacylphosphatidylethanolamine comprising dimyristoyl (C14) chain, Dipalmitoyl (C16) chain or Distearoyl (C18) chain, and optionally the free PEG-lipid comprises PEG-DPG and PEG-DMG.
B100. The LNP of any one of embodiments 81 to 99, wherein the free PEG-lipid is present in the lipid blend in a range of about 1 to about 4 mol %, such as about 0.5 to about 3 mol %.
B101. The LNP of embodiment 100, wherein the free PEG-lipid is present in the lipid blend in a range of about 2.4 mol % to about 2.6 mol %.
B102. The LNP of any one of embodiments 81 to 101, wherein the free PEG-lipid comprises the same or a different lipid as the lipid in the lipid-immune cell targeting group conjugate.
B103. The LNP of any one of embodiments 62 to 102, wherein the LNP has a mean diameter in the range of 50-200 nm.
B104. The LNP of embodiment 103, wherein the LNP has a mean diameter of between about 75 nm and about 80 nm.
B105. The LNP of any one of embodiments 62 to 104, wherein the LNP has a polydispersity index in a range from about 0.01 to about 0.5.
B106. The LNP of any one of embodiments 62 to 105, wherein the LNP has a pKa of between about 5.0 and about 8.0.
B107. The LNP of any one of embodiments 62 to 106, wherein the nucleic acid is DNA or RNA.
B108. The LNP of embodiment 107, wherein the RNA is an mRNA.
B109. The LNP of embodiment 108, wherein the mRNA encodes a receptor, a growth factor, a hormone, a cytokine, an antibody, an antigen, an enzyme, or a vaccine.
B110. The LNP of embodiment 108, wherein the mRNA encodes a polypeptide capable of regulating immune response in the immune cell.
B111. The LNP of embodiment 108, wherein the mRNA encodes a polypeptide capable of reprogramming the immune cell.
B112. The LNP of embodiment 108, wherein the mRNA encodes a synthetic T cell receptor (synTCR) or a Chimeric Antigen Receptor (CAR).
B113. The LNP of embodiment 108, wherein the mRNA encodes polypeptide capable of reprogramming an M2 macrophage to an M1 macrophage.
B114. The LNP of embodiment 108, wherein the RNA is tRNA, siRNA, gRNA, or microRNA.
B115. The LNP of any one of embodiments 62 to 107, wherein the immune cell targeting group comprises an antibody, and the antibody is a Fab or an immunoglobulin single variable (ISV) domain (e.g., a Nanobody).
B116. The LNP of embodiment 115, wherein the antibody is a human or humanized antibody.
B117. The LNP of any one of embodiments 62 to 116, wherein the immune cell targeting group comprises a Fab, F(ab′)₂, Fab′-SH, Fv, or scFv fragment, or any combination thereof.
B118. The LNP of any one of embodiments 62 to 117, wherein the immune cell targeting group comprises a Fab.
B119. The LNP of embodiment 118, wherein the Fab is engineered to knock out the natural interchain disulfide bond at the C-terminus.
B120. The LNP of embodiment 119, wherein the Fab comprises a heavy chain fragment that comprises C233S substitution, and a light chain fragment that comprises C214S substitution, numbering according to Kabat.
B121. The LNP of embodiment 118, wherein the Fab has a non-natural interchain disulfide bond (e.g., an engineered, buried interchain disulfide bond).
B122. The LNP of embodiment 121, wherein the Fab comprises F174C substitution in the heavy chain fragment, and S176C substitution in the light chain fragment, numbering according to Kabat.
B123. The LNP of embodiment 118, wherein the Fab comprises a cysteine at the C-terminus of the heavy or light chain fragment.
B124. The LNP of embodiment 123, wherein the Fab further comprises one or more amino acids between the heavy chain fragment of the Fab and the C-terminal cysteine.
B125. The LNP of embodiment 118, wherein the Fab comprises a heavy chain variable domain linked to an antibody CH₁ domain and a light chain variable domain linked to an antibody light chain constant domain, wherein the CH₁ domain and the light chain constant domain are linked by one or more interchain disulfide bonds, and wherein the immune cell targeting group further comprises a single chain variable fragment (scFv) linked to the C-terminus of the light chain constant domain by an amino acid linker.
B126. The LNP of embodiment 115 or 116, wherein the immune cell targeting group comprises an ISV domain.
B127. The LNP of embodiment 126, wherein the ISV domain is Nanobody® ISV.
B128. The LNP of embodiment 126 or 127, wherein the ISV domain comprises a cysteine at the C-terminus.
B129. The LNP of embodiment 128, wherein the ISV domain comprises a V_{HH} domain and further comprises a spacer comprising one or more amino acids between the V_{HH} domain and the C-terminal cysteine.
B130. The LNP of any one of embodiments 126 to 129, wherein the immune cell targeting group comprises two or more Van domains.
B131. The LNP of embodiment 130, wherein the two or more V_{HH} domains are linked by an amino acid linker.
B132. The LNP of embodiment 130, wherein the immune cell targeting group comprises a first V_{HH} domain linked to an antibody CH₁ domain and a second Van domain linked to an antibody light chain constant domain.
B133. The LNP of embodiment 132, wherein the antibody CH₁ domain and the antibody light chain constant domain are linked by one or more disulfide bonds.
B134. The LNP of any one of embodiments 126 to 130, wherein the immune cell targeting group comprises a Van domain linked to an antibody CH₁ domain, and wherein the antibody CH₁ domain is linked to an antibody light chain constant domain by one or more disulfide bonds.
B135. The LNP of embodiment 133 or 134, wherein the CH₁ domain comprises F174C and C233S substitutions, and the light chain constant domain comprises S176C and C214S substitutions, numbering according to Kabat.
B136. The LNP of any one of embodiments 62 to 135, wherein the immune cell targeting group comprises a Fab that comprises:
   (a) a heavy chain fragment comprising the amino acid sequence of SEQ ID NO: 1 and a light chain fragment comprising the amino acid sequence of SEQ ID NO: 2 or 3; or
   (b) a heavy chain fragment comprising the amino acid sequence of SEQ ID NO: 6 and a light chain fragment comprising the amino acid sequence of SEQ ID NO: 7.
B137. The LNP of any one of embodiments 62 to 136, wherein the LNP is for delivering a nucleic acid into an immune cell, and wherein the immune cell is an NK cell, and the immune cell targeting group comprises an antibody that binds CD56.
B138. The LNP of any one of embodiments 81 to 136, wherein the LNP is for delivering a nucleic acid into an immune cell, and wherein the immune cell targeting group comprises an antibody that binds CD7 or CD8, and the free PEG-lipid is DMG-PEG or PEG-DPG.
B139. The LNP of any one of embodiments 62 to 136, wherein the LNP is for delivering a nucleic acid into an immune cell, and wherein the immune cell is a macrophage, and the immune cell targeting group comprises an antibody that binds CD206.
B140. The LNP of any one of embodiments 81 to 136, wherein the LNP is for delivering a nucleic acid into an immune cell, and wherein the immune cell targeting group comprises an antibody that binds CD206, and the free PEG-lipid is DMG-PEG or PEG-DPG.
B141. The LNP of any one of embodiments 81 to 140, wherein the free PEG-lipid comprises a PEG having a molecular weight of at least 2000 daltons.
B142. The LNP of embodiment 134, wherein the PEG has a molecular weight of about 3000 to 5000 daltons.
B143. The LNP of any one of embodiments 115 to 142, wherein the antibody is a Fab.
B144. The LNP of embodiment 143, wherein the Fab binds CD3, and the free PEG-lipid in the LNP comprises a PEG having a molecular weight of about 2000 daltons.
B145. The LNP of embodiment 143, wherein the Fab is an anti-CD4 antibody, and the free PEG-lipid in the LNP comprises a PEG having a molecular weight of about 3000 to 3500 daltons.
B146. The LNP of embodiment 143, wherein the Fab binds CD206, and the free PEG-lipid in the LNP comprises a PEG having a molecular weight of about 2000 daltons.
B147. The LNP of embodiment 143, wherein the Fab is an anti-CD206 antibody, and the free PEG-lipid in the LNP comprises a PEG having a molecular weight of about 3000 to 3500 daltons.
B148. The LNP of any one of embodiments 62 to 135, wherein the LNP is for delivering a nucleic acid into an immune cell, and wherein the LNP binds CD3, and also binds CD11a or CD18 of the immune cell.
B149. The LNP of embodiment 148, wherein the LNP comprises two conjugates, wherein the first conjugate comprises an antibody that binds CD3, and the second conjugate comprises an antibody that binds CD11a or CD18.
B150. The LNP of embodiment 148, wherein the LNP comprises one conjugate, and the conjugate comprises a bispecific antibody that binds both CD3 and CD11a.
B151. The LNP of embodiment 148, wherein the LNP comprises one conjugate, and the conjugate comprises a bispecific antibody that binds both CD3 and CD18.
B152. The LNP of embodiment 150 or 151, wherein the bispecific antibody is an immunoglobulin single variable domain or Fab-ScFv.
B153. The LNP of any one of embodiments 62 to 136, wherein the LNP is for delivering a nucleic acid into an immune cell, and wherein the LNP binds CD7 and CD8 of the immune cell.
B154. The LNP of embodiment 153, wherein the LNP comprises two conjugates, wherein the first conjugate comprises an antibody that binds CD7, and a second conjugate that binds CD8
B155. The LNP of embodiment 153, wherein the LNP comprises one conjugate, wherein the conjugate comprises a bispecific antibody that binds CD7 and CD8.
B156. The LNP of embodiment 155, wherein the bispecific antibody is an immunoglobulin single variable domain or Fab-ScFv.
B157. The LNP of any one of embodiments 62 to 136, wherein the LNP is for delivering a nucleic acid into an immune cell, and wherein the LNP binds a first macrophage antigen, and also binds a second macrophage antigen.
B158. The LNP of embodiment 157, wherein the LNP comprises two conjugates, wherein the first conjugate comprises an antibody that binds the first macrophage antigen, and the second conjugate comprises an antibody that binds the second macrophage antigen.
B159. The LNP of embodiment 157, wherein the LNP comprises one conjugate, and the conjugate comprises a bispecific antibody that binds both the first macrophage antigen and the second macrophage antigen.
B160. The LNP of embodiment 159, wherein the bispecific antibody is an immunoglobulin single variable domain or Fab-ScFv.
B161. The LNP of any one of embodiments 62 to 136, wherein the LNP binds to a first antigen on the surface of the first type of immune cell, and also binds to a second antigen on the surface of the second type of immune cell.
B162. The LNP of embodiment 161, wherein the two different types of immune cells are CD4+ T cells and CD8+ T cell.
B163. The LNP of embodiment 161, wherein the first type of immune cell is a first macrophage, and the second type of immune cell is a second macrophage, a T-cell, or an NK cell.
B164. The LNP of embodiment 161, wherein the LNP comprises two conjugates, and the first conjugate comprises a first antibody that binds to the first antigen of the first type of immune cell, and the second conjugate comprises a second antibody that binds to the second antigen of the second type of immune cell.
B165. The LNP of embodiment 161, wherein the LNP comprises one conjugate, and the conjugate comprises a bispecific antibody, and the bispecific antibody binds to both the first antigen on the first type of immune cell, and the second antigen on the second type of immune cells.
B166. The LNP of any one of embodiments 62 to 136, wherein the bispecific antibody is an immunoglobulin single variable domain or a Fab-ScFv.
B167. The LNP of any one of embodiments 62 to 136, wherein the LNP is for delivering a nucleic acid into an immune cell, and wherein the immune cell targeting group comprises a single antibody that binds to CD3 or CD7.
B168. The LNP of any one of embodiments 62 to 136, wherein the LNP is for delivering a nucleic acid into an immune cell, and wherein the immune cell targeting group binds to CD7, CD8, or both CD7 and CD8.
B169. The LNP of any one of embodiments 62 to 136, wherein the LNP is for delivering a nucleic acid into both T cells and NK cells, wherein the immune cell targeting group binds to
   (a) both CD3 and CD56;
   (b) both CD8 and CD56; or
   (c) both CD7 and CD56.
B170. The LNP of any one of embodiments 62 to 136, wherein the LNP is for delivering a nucleic acid into an immune cell, and wherein the immune cell targeting group comprises a Fab lacking the native interchain disulfide bond.
B171. The LNP of embodiment 170, wherein the Fab is engineered to replace one or both cysteines on the native constant light chain and the native constant heavy chain that form the native interchain disulfide with a non-cysteine amino acid, therefor to remove the native interchain disulfide bond in the Fab.
B172. A method of targeting the delivery of a nucleic acid to an immune cell of a subject, the method comprising contacting the immune cell with the LNP of any one of embodiments 62 to 171, wherein the LNP comprises the nucleic acid.
B173. A method of expressing a polypeptide of interest in a targeted immune cell of a subject, the method comprising contacting the immune cell with the LNP of any one of embodiments 62 to 171, wherein the LNP comprises a nucleic acid encoding the polypeptide.
B174. A method of modulating cellular function of a target immune cell of a subject, the method comprising administering to the subject the LNP of any one of embodiments 62 to 171, wherein the LNP comprises a nucleic acid modulates the cellular function of the immune cell.
B175. A method of treating, ameliorating, or preventing a symptom of a disorder or disease in a subject, the method comprising administering to the subject an LNP of any one of embodiments 62 to 171, wherein the LNP comprises a nucleic acid and delivers the nucleic acid into an immune cell of the subject.
B176. The method of embodiment 175, wherein the disorder is an immune disorder, an inflammatory disorder, or cancer.
B177. The method of embodiment 175, wherein the nucleic acid encodes an antigen for use in a therapeutic or prophylactic vaccine for treating or preventing cancer or an infection by a pathogen.
B178. The method of any one of embodiments 172 to 177, wherein no more than 5% non-immune cells are transfected by the LNP.
B179. The method of any one of embodiments 172 to 178, wherein half-life of the nucleic acid delivered by the LNP or a polypeptide encoded by the nucleic acid delivered by the LNP is at least 10% longer than half-life of nucleic acid delivered by a reference LNP or a polypeptide encoded by the nucleic acid delivered by the reference LNP.
B180. The method of any one of embodiments 172 to 179, wherein at least 10% immune cells are transfected by the LNP.
B181. The method of any one of embodiments 172 to 180, wherein expression level of the nucleic acid delivered by the LNP is at least 10% higher than expression level of nucleic acid delivered by a reference LNP.
B182. The method of any one of embodiments 172 to 181, wherein the LNP comprises the compound of any one of embodiments 1 to 61, or a salt thereof.
B183. The method of embodiment 182, wherein (i) R^{c3} is

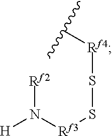

or (ii) R^{a3} and R^{b3} are each independently

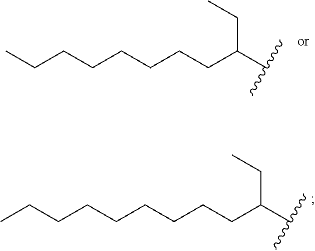 [SMILES: *C(CC)CCCCCCCC ; *C(CC)CCCCCCCCC]

or both (i) and (ii).
B184. The method of embodiment 182, wherein the LNP comprises Lipid 28, Lipid 6, Lipid 12, Lipid 1, or Lipid 7 of Table 1, or a salt thereof, or any combination thereof.
B185. The method of embodiment 182, wherein (i) R^{a3} and R⁵³ are each independently

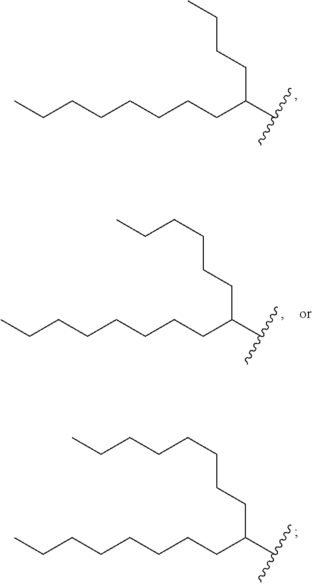 [SMILES: *C(CCCC)CCCCCCCC ; *C(CCCCCC)CCCCCCCC ; *C(CCCCCCCC)CCCCCCCC]

or at least one of R^{a3} and R^{b3} is

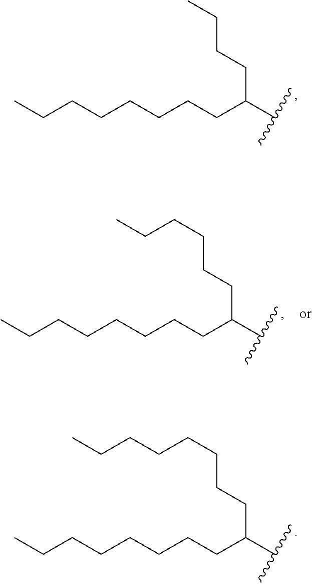 [SMILES: *C(CCCC)CCCCCCCC ; *C(CCCCCC)CCCCCCCC ; *C(CCCCCCCC)CCCCCCCC]

B186. The method of embodiment 182, wherein the LNP comprises Lipid 9 or Lipid 10 of Table 1, or a salt thereof, or any combination thereof.
B187. A method of targeting the delivery of a nucleic acid to a non-liver cell, the method comprising contacting the non-liver cell with an LNP comprising the compound of any one of embodiments 1-61, or a salt thereof, wherein (i) R^{c3} is

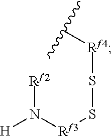

or (ii) R^{a3} and R^{b3} are each independently

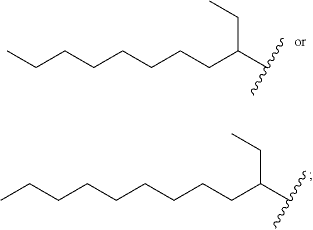 [SMILES: *C(CC)CCCCCCCC ; *C(CC)CCCCCCCCC]

or both (i) and (ii).
B188. The method of embodiment 187, wherein the LNP comprises Lipid 28, Lipid 6, Lipid 12, Lipid 1, or Lipid 7 of Table 1, or a salt of thereof, or any combination thereof.
B189. A method of targeting the delivery of a nucleic acid to a liver cell, the method comprising contacting the liver cell with an LNP comprising the compound of any one of embodiments 1-61, or a salt thereof, wherein (i) R^{a3} and R^{b3} are each independently

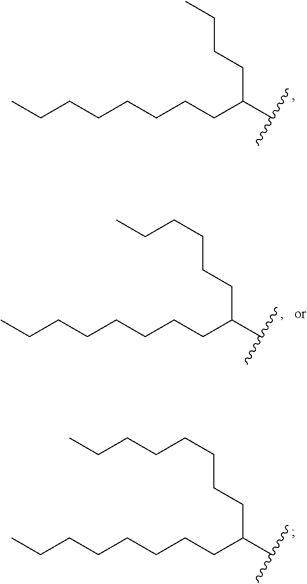 [SMILES: *C(CCCC)CCCCCCCC ; *C(CCCCCC)CCCCCCCC ; *C(CCCCCCCC)CCCCCCCC]

or at least one of R³³ and R^{b3} is

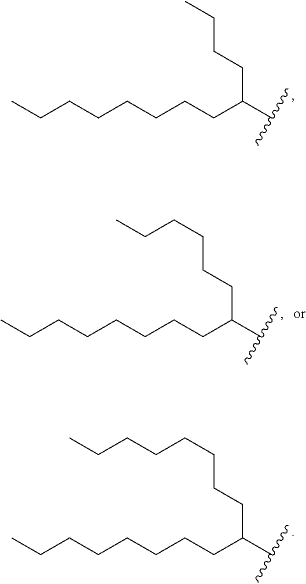 [SMILES: *C(CCCC)CCCCCCCC ; *C(CCCCCC)CCCCCCCC ; *C(CCCCCCCC)CCCCCCCC]

B190. The method of embodiment 189, wherein the LNP comprises Lipid 9 or Lipid 10 of Table 1, or a salt thereof, or any combination thereof.
B191. A method of targeting the delivery of a nucleic acid to a placental cell, the method comprising contacting the placental cell with an LNP comprising the compound of any one of embodiments 1-61, or a salt thereof.
B192. A lipid nanoparticle (LNP) comprising a lipid blend for targeted delivery of a nucleic acid into a hematopoietic stem cell (HSC), the lipid blend comprising a lipid-cell targeting group conjugate comprising the compound of Formula (V): [Lipid]-[optional linker]-[cell targeting group] and an ionizable cationic lipid selected from a compound of any one of embodiments 1-61, or a salt thereof, wherein the cell targeting group is an antibody that binds to an antigen on the HSC, and wherein the nucleic acid is encapsulated in the LNP.
B193. The LNP of embodiment 192, wherein the antigen on the hematopoietic stem cell is selected from the group consisting of CD34, CD105, and CD117.
B194. The LNP of embodiment 192 or 193, wherein the LNP comprises Lipid 1, Lipid 12, or Lipid 53 of Table 1, or a salt thereof, or any combination thereof.
B195. The LNP of any one of embodiments 192 to 194, wherein the ionizable cationic lipid is present in the lipid blend in a range of 30-70 (e.g., 40-60) mol %.
B196. The LNP of embodiment 195, wherein the ionizable cationic lipid is present in the lipid blend in a range of about 48 mol % to about 50 mol %.
B197. The LNP of any one of embodiments 192 to 196, wherein the cell targeting group is covalently coupled to a lipid in the lipid blend via a polyethylene glycol (PEG) containing linker.
B198. The LNP of embodiment 197, wherein the lipid covalently coupled to the cell targeting group via a PEG containing linker is distearoylglycerol (DSG), distearoyl-phosphatidylethanolamine (DSPE), dimyristoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide,
B199. The LNP of embodiment 197 or 198, wherein the PEG is PEG 3400.
B200. The LNP of any one of embodiments 192 to 199, wherein the lipid covalently coupled to the cell targeting group via a PEG containing linker is distearoyl-phosphatidylethanolamine (DSPE).
B201. The LNP of any one of embodiments 192 to 200, wherein the [Lipid]-[optional linker]-[cell targeting group] conjugate is present in the lipid blend in a range of 0.001 to 0.5 mole percent (e.g., 0.002-0.2 mole percent).
B202. The LNP of any one of embodiments 192 to 201, wherein the lipid blend further comprises one or more of a structural lipid, a neutral phospholipid, and a free PEG-lipid.
B203. The LNP of embodiment 202, wherein the structural lipid is present in the lipid blend in a range of 20-70 (e.g., 30-50) mol %.
B204. The LNP of embodiment 203, wherein the structural lipid is present in the lipid blend in a range of about 27 mol % to about 29 mol %.
B205. The LNP of any one of embodiments 202 to 204, wherein the structural lipid is sterol.
B206. The LNP of embodiment 205, wherein the sterol is cholesterol, fecosterol, β-sitosterol, ergosterol, campesterol, stigmasterol, stigmastanol, or brassicasterol.
B207. The LNP of embodiment 205, wherein the sterol is cholesterol.
B208. The LNP of any one of embodiments 202 to 207, wherein the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin.
B209. The LNP of any one of embodiments 202 to 207, wherein the neutral phospholipid is 1,2-distearoyl-sn-glycero-3-phosphocholine (DSPC).
B210. The LNP of any one of embodiments 202 to 209, wherein the neutral phospholipid is present in the lipid blend in a range of 5-15 mol %.
B211. The LNP of any one of embodiments 202 to 209, wherein the neutral phospholipid is present in the lipid blend in a range of about 16 mol % to about 40 mol %
B212. The LNP of any one of embodiments 202 to 209, wherein the neutral phospholipid is present in the lipid blend in a range of about 16 mol % to about 30 mol %.
B213. The LNP of any one of embodiments 202 to 209, wherein the neutral phospholipid is present in the lipid blend in a range of about 16 mol % to about 25 mol %.
B214. The LNP of any one of embodiments 202 to 209, wherein the neutral phospholipid is present in the lipid blend in a range of about 19 mol % to about 21 mol %.
B215. The LNP of any one of embodiments 202 to 209, wherein the neutral phospholipid is present in the lipid blend in about 20 mol %.
B216. The LNP of any one of embodiments 202 to 215, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, for example, a PEG lipid may be PBG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyristoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.
B217. The LNP of any one of embodiments 202 to 215, wherein the free PEG-lipid is DPG-PEG2K.
B218. The LNP of any one of embodiments 202 to 217, wherein the free PEG-lipid is present in the lipid blend in a range of about 1 to about 4 mol %, such as about 0.5 to about 3 mol %.
B219. The LNP of embodiment 218, wherein the free PEG-lipid is present in the lipid blend in a range of about 2.4 mol % to about 2.6 mol %.
B220. The LNP of any one of embodiments 192 to 219, wherein the cell targeting group comprises an antibody, and the antibody is a Fab or an immunoglobulin single variable (ISV) domain (e.g., a Nanobody).
B221. The LNP of any one of embodiments 192 to 219, wherein the cell targeting group comprises a Fab, F(ab′)₂, Fab′-SH, Fv, or scFv fragment, or any combination thereof.
B222. The LNP of embodiment 220, wherein the cell targeting group comprises an ISV domain.
B223. The LNP of embodiment 220, wherein the cell targeting group comprises a Fab.
B224. A method of targeting the delivery of a nucleic acid to a hematopoietic stem cell (HSC), the method comprising contacting the HSC with an LNP comprising the compound of any one of embodiments 1 to 61, or a salt thereof, or an LNP of any one of embodiments 192 to 223.
B225. A method of genetically modifying a hematopoietic stem cell (HSC), the method comprising contacting the HSC with an LNP comprising the compound of any one of embodiments 1 to 61 or a salt thereof, or an LNP of any one of embodiments 192 to 223.
B226. A method of treating a disease in a subject in need thereof, the method comprising administering to the subject an LNP of any one of embodiments 192 to 223.

### EXAMPLES

The invention now being generally described, will be more readily understood by reference to the following examples, which are included merely for purposes of illustration of certain aspects and embodiments of the present invention, and are not intended to limit the invention.

### Example 1. Preparation of Ionizable Cationic Lipids

This Example describes the synthesis of various cationic lpids.

### Synthesis of Lipid 1

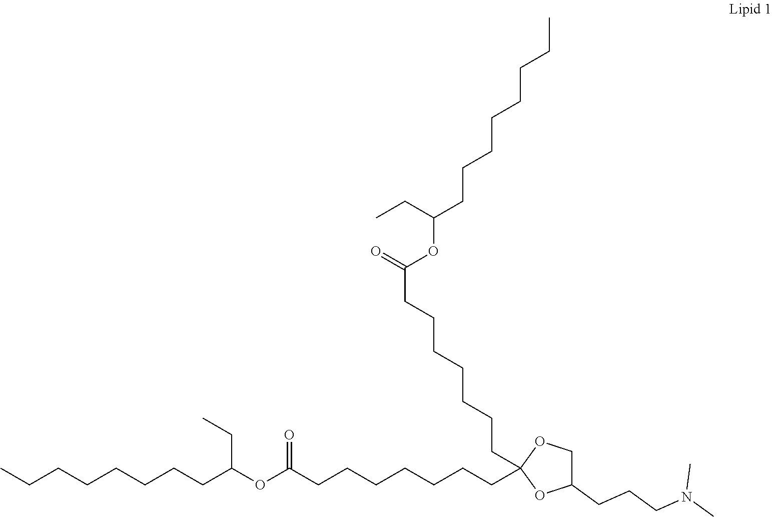 [SMILES: CCCCCCCCC(CC)OC(=O)CCCCCCCC1(CCCCCCCC(=O)OC(CC)CCCCCCCC)OCC(CCCN(C)C)O1]

A scheme for the synthesis of Lipid 1 is provided in Scheme 1 below.

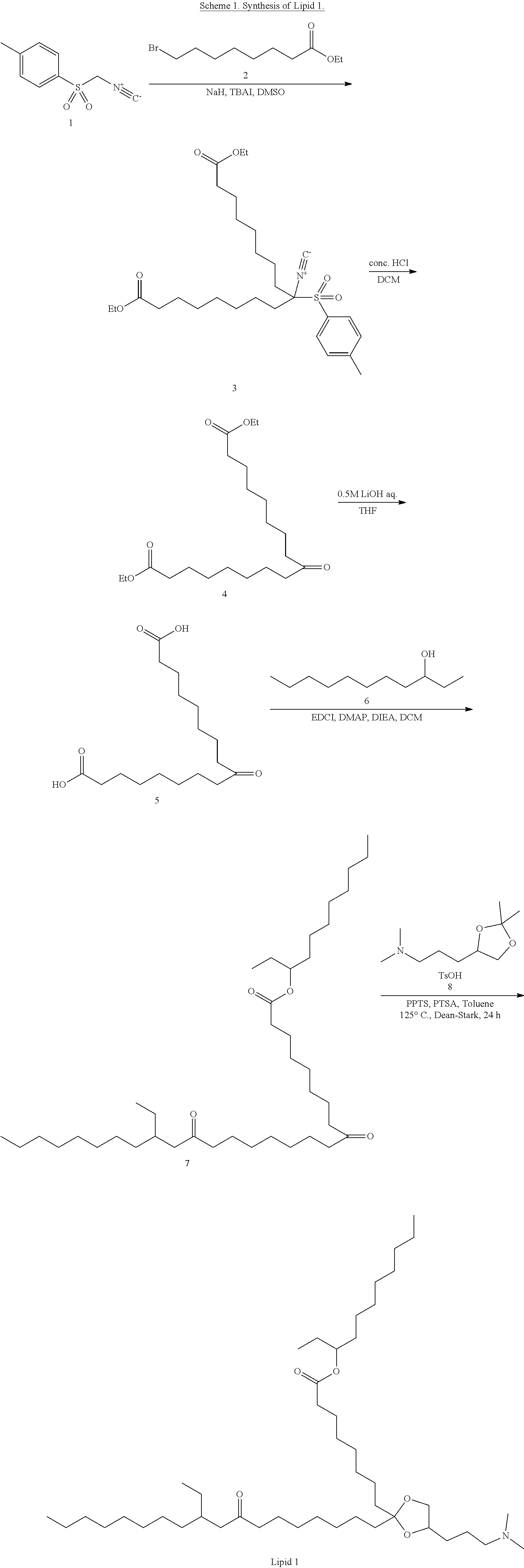

### Procedure for Preparation of Compound 3

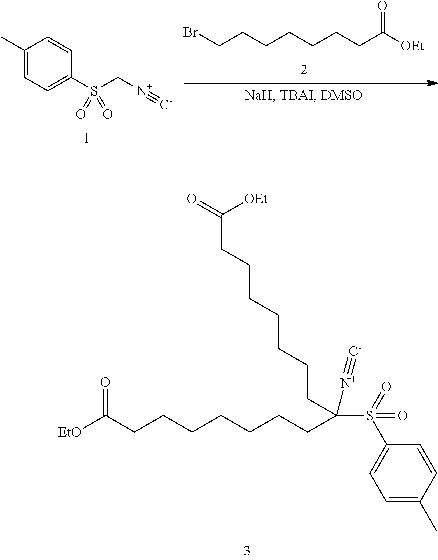

To DMSO (500 mL) was added NaH (6.40 g, 160.06 mmol, 60% purity, 2.5 eq.) in portions at 25° C. and then the mixture was stirred at 25° C. for 1 hour. And then TOSMIC (12.5 g, 64.02 mmol, 1 eq.) was added in portions, followed by TBAI (2.36 g, 6.40 mmol, 0.1 eq). After stirring at 25° C. for additional 15 mins, compound 2 (33.61 g, 133.81 mmol, 2.09 eq.) was added portionwise, and then the mixture was stirred at 25° C. for 1 hour under N₂ atmosphere. The mixture was poured into water (60 mL) and extracted with heptane (120 mL). The organic layer was washed with brine (60 mL), dried over anhydrous Na₂SO₄, filtered and concentrated in vacuo to give crude compound 3 (45 g) as a yellow oil, which was used directly for next step without further purification.

### Procedure for Preparation of Compound 4

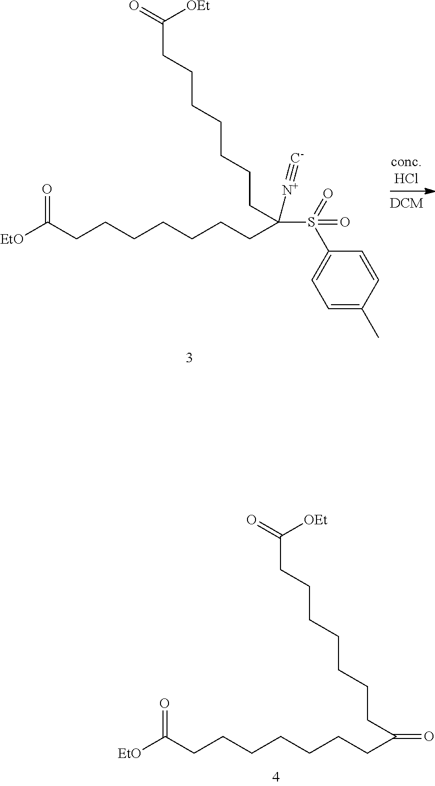 [SMILES: [C-]#[N+]C(CCCCCCCC(=O)OCC)(CCCCCCCC(=O)OCC)S(=O)(=O)c1ccc(C)cc1 ; CCOC(=O)CCCCCCCC(=O)CCCCCCCC(=O)OCC]

To a solution of compound 3 (45 g, 84.00 mmol, 1 eq.) in DCM (240 mL) was added HCl (concentrated, 60 mL) at 25° C. The mixture was stirred at 25° C. for 2 hours. The mixture was poured into water (60 mL) and extracted with DCM (120 mL). The organic layer was washed with brine (60 mL), dried over anhydrous Na₂SO₄, filtered and concentrated in vacuo to give crude compound 4 (38 g) as a yellow solid, which was used directly for next step without further purification.

### Procedure for Preparation of Compound 5

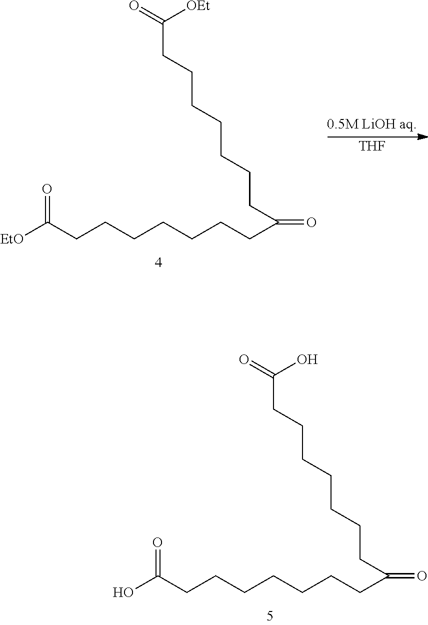 [SMILES: CCOC(=O)CCCCCCCC(=O)CCCCCCCC(=O)OCC ; O=C(O)CCCCCCCC(=O)CCCCCCCC(=O)O ; "THF"]

To a solution of compound 4 (38 g, 102.56 mmol, 1 eq.) in THF (300 mL) was added LiOH (0.5 M aqueous solution, 861.49 mL, 4.2 eq.) at 25° C. The mixture was stirred at 25° C. for 12 hours. The reaction mixture was concentrated under reduced pressure to remove the organic solvent. The remaining aqueous solution was washed with (Petroleum ether/Ethyl acetate=10:1, 125 mL), and then acidified with 1 M HCl aqueous solution (˜240 mL) to pH=4 and stirred for 30 mins. The precipitate was filtered out and dried in vacuo to give compound 5 (24 g, crude) as a white solid.

¹HNMR: (400 MHz, DMSO-d6) δ ppm 12.71-11.10 (m, 2H), 2.38 (t, J=7.3 Hz, 4H), 2.24-2.13 (m, 4H), 1.54-1.36 (m, 8H), 1.30-1.15 (m, 12H).

### Procedure for Preparation of Compound 7

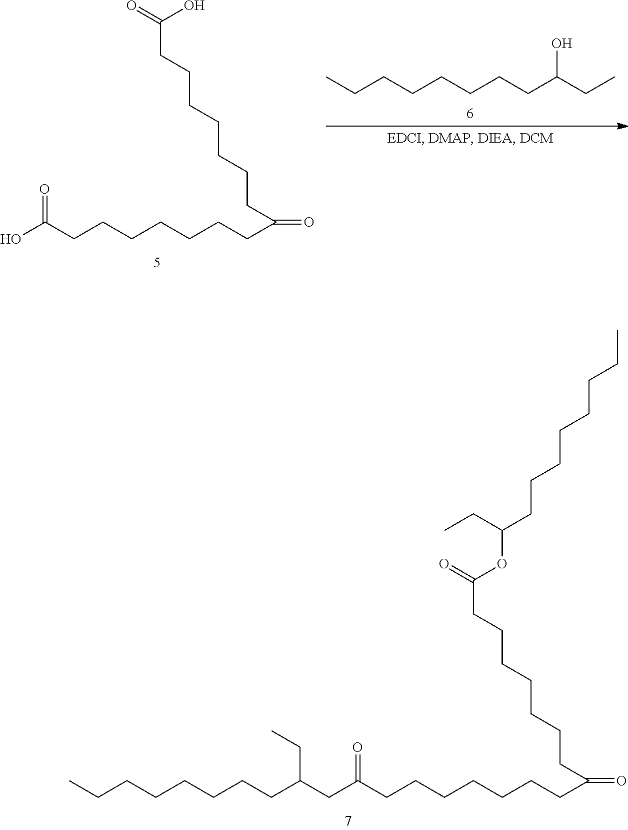 [SMILES: O=C(O)CCCCCCCC(=O)CCCCCCCC(=O)O ; CCCCCCCCC(O)CC ; CCCCCCCCC(CC)CC(=O)CCCCCCCC(=O)CCCCCCCC(=O)OC(CC)CCCCCCCC]

To a solution of compound 5 (24 g, 76.33 mmol, 1 eq.) and compound 6 (31.57 g, 183.20 mmol, 2.4 eq.) in DCM (250 mL) was added EDCI (58.53 g, 305.33 mmol, 4 eq.), DIEA (88.79 g, 686.99 mmol, 9 eq.) and DMAP (9.33 g, 76.33 mmol, 1 eq.) at 25° C. The mixture was stirred at 40° C. for 12 hours. The reaction mixture was concentrated under reduced pressure. The residue was purified by column chromatography (SiO2, Petroleum ether/Ethyl acetate=20/1 to 10/1) to give compound 7 (22.1 g, 46.47% yield) as a light-yellow oil.

¹HNMR: (400 MHz, CHLOROFORM-d) δ ppm 4.83 (m, 2H), 2.39 (t, J=7.4 Hz, 4H), 2.30 (t, J=7.5 Hz, 4H), 1.67-1.50 (m, 16H), 1.38-1.22 (m, 36H), 0.94-0.85 (m, 12H).

### Procedure for Preparation of Lipid 1

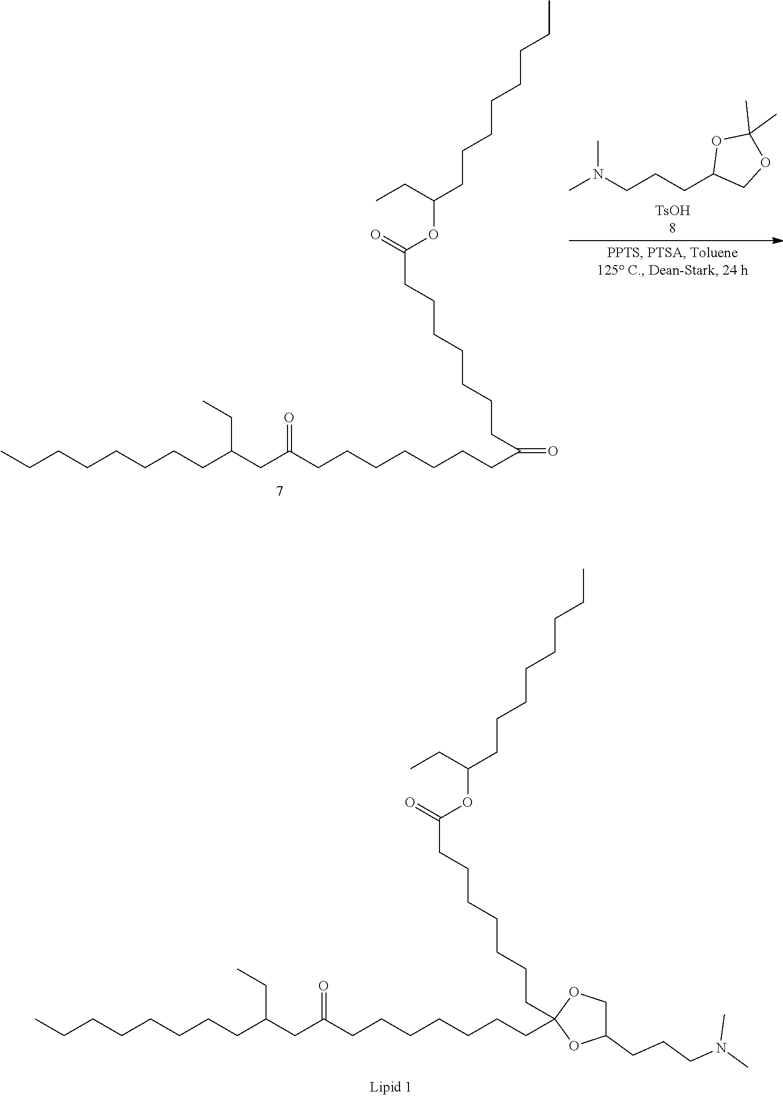

To a solution of compound 7 (3 g, 4.82 mmol, 1 eq.) and compound 8 (TsOH salt, 5.19 g, 14.45 mmol, 3 eq.) in toluene (30 mL) was added PPTS (1.21 g, 4.82 mmol, 1 eq.) and PTSA (457.99 mg, 2.41 mmol, 0.5 eq.) at 25° C. The mixture was stirred at 125° C. for 12 hours. After cooled to room temperature, the reaction mixture was poured into Sat. NaHCO₃ aqueous solution (20 mL) and stirred for 2 mins. The aqueous phase was extracted with DCM (50 mL*3). The combined organic phase was washed with brine (60 mL), dried over anhydrous Na₂SO₄, filtered and concentrated in vacuo. The residue was purified by column chromatography (SiO2, DCM/MeOH=20/1 to 10/1) to give Lipid 1 (1.53 g, 41.82% yield, 99% purity) as a yellow oil.

LCMS: m/z 752.9 [M+1]+. ¹HNMR: (400 MHz, CHLOROFORM-d) δ ppm 4.85 (m, 2H), 4.15-4.01 (m, 2H), 3.56-3.42 (m, 1H), 2.40-2.30 (m, 6H), 2.30-2.27 (m, 6H), 1.65-1.50 (m, 20H), 1.39-1.25 (m, 40H), 0.95-0.87 (m, 12H).

### Synthesis of Lipid 2

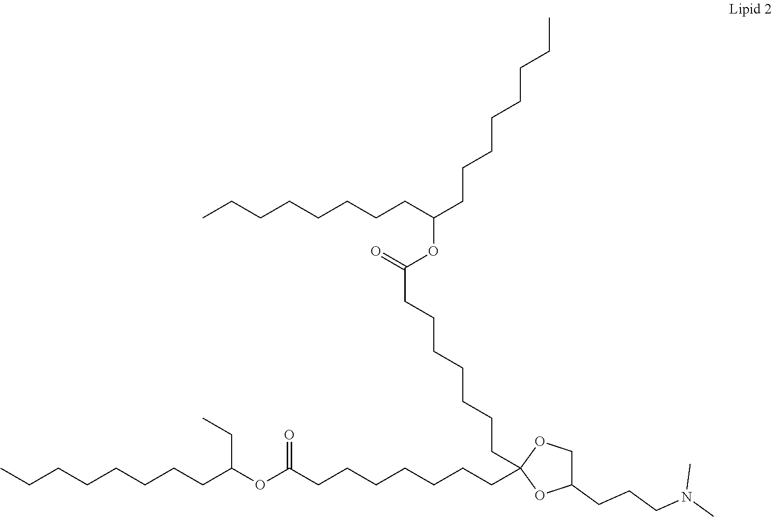 [SMILES: CCCCCCCCC(CC)OC(=O)CCCCCCCC1(CCCCCCCC(=O)OC(CCCCCCCC)CCCCCCCC)OCC(CCCN(C)C)O1]

A scheme for the synthesis of Lipid 2 is provided in Scheme 2 below.

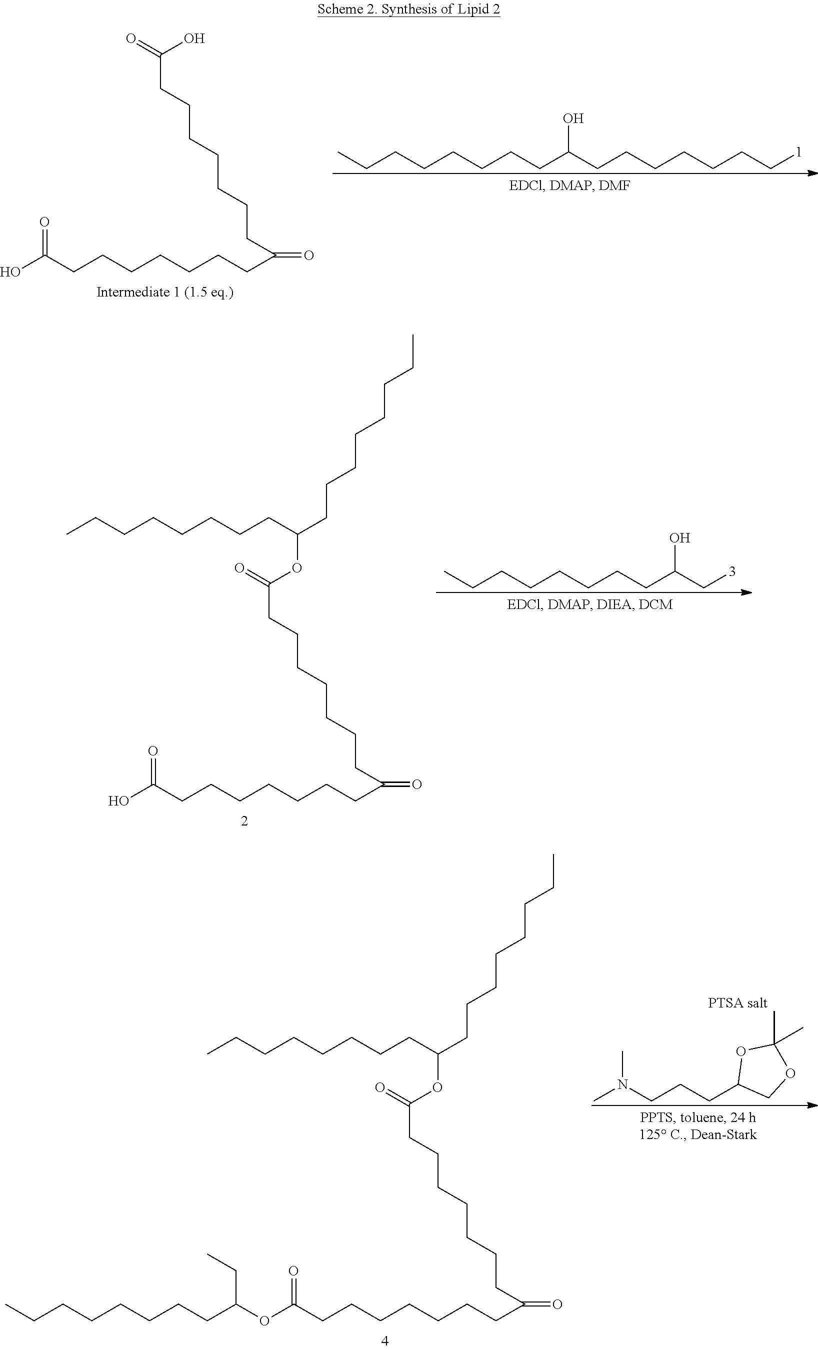

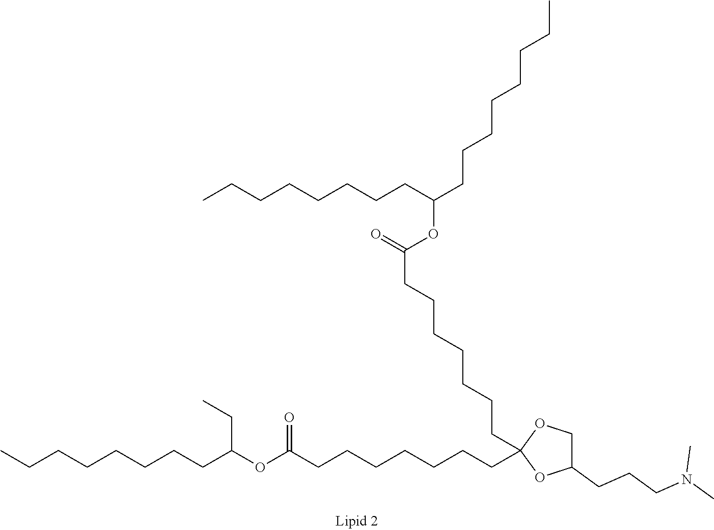 [SMILES: CCCCCCCCC(CC)OC(=O)CCCCCCCC1(CCCCCCCC(=O)OC(CCCCCCCC)CCCCCCCC)OCC(CCCN(C)C)O1]

### Procedure for Preparation of Compound 2

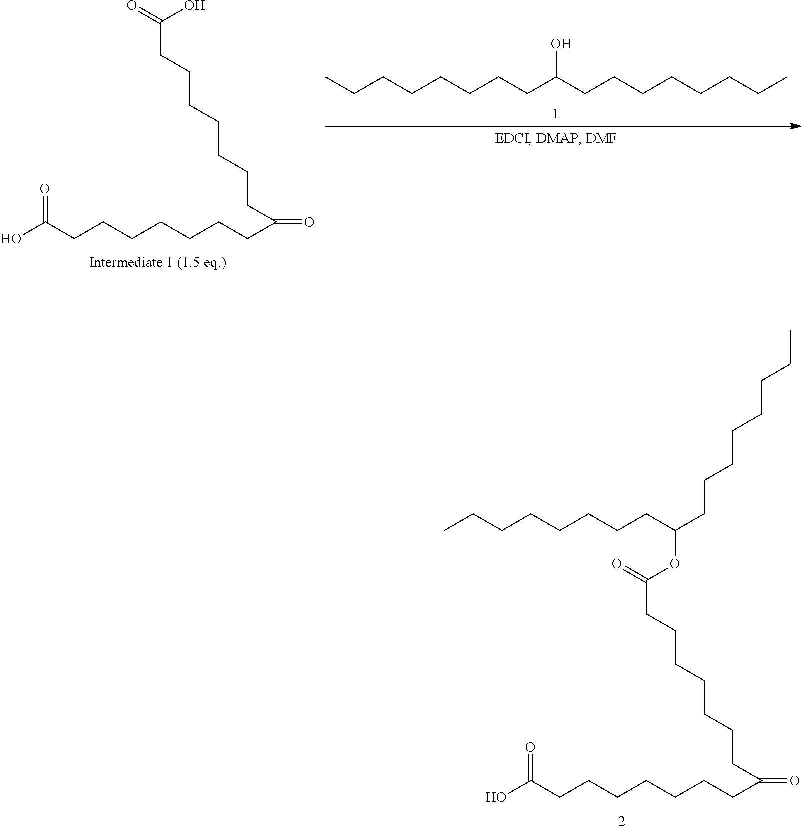 [SMILES: O=C(O)CCCCCCCC(=O)CCCCCCCC(=O)O ; CCCCCCCCC(O)CCCCCCCC ; CCCCCCCCC(CCCCCCCC)OC(=O)CCCCCCCC(=O)CCCCCCCC(=O)O]

To a solution of intermediate 1 (7.36 g, 23.39 mmol, 1.5 eq.) and compound 1 (4 g, 15.60 mmol, 1 eq.) in DMF (100 mL) was added DMAP (1.96 g, 16.06 mmol, 1.03 eq.) and EDCI (3.92 g, 20.43 mmol, 1.31 eq.) at 25° C. The mixture was stirred at 25° C. for 12 hours. The residue was poured into ice-water (w/w=1/1, 100 mL) and stirred for 2 mins. The aqueous phase was extracted with DCM (40 mL*3). The combined organic phases were washed with brine (60 mL), dried over anhydrous Na₂SO₄, filtered and concentrated under vacuum. The residue was purified by silica gel column chromatography (SiO₂, Petroleum ether/Ethyl acetate=4/1 to 2/1) to give compound 2 (4.5 g, 52.19% yield) as a white solid.

¹HNMR: (400 MHz, CHLOROFORM-d) δ ppm 4.87 (t, J=6.4 Hz, 1H), 2.47-2.15 (m, 8H), 1.69-1.46 (m, 12H), 1.38-1.21 (m, 36H), 0.88 (t, J=6.8 Hz, 6H).

### Procedure for Preparation of Compound 4

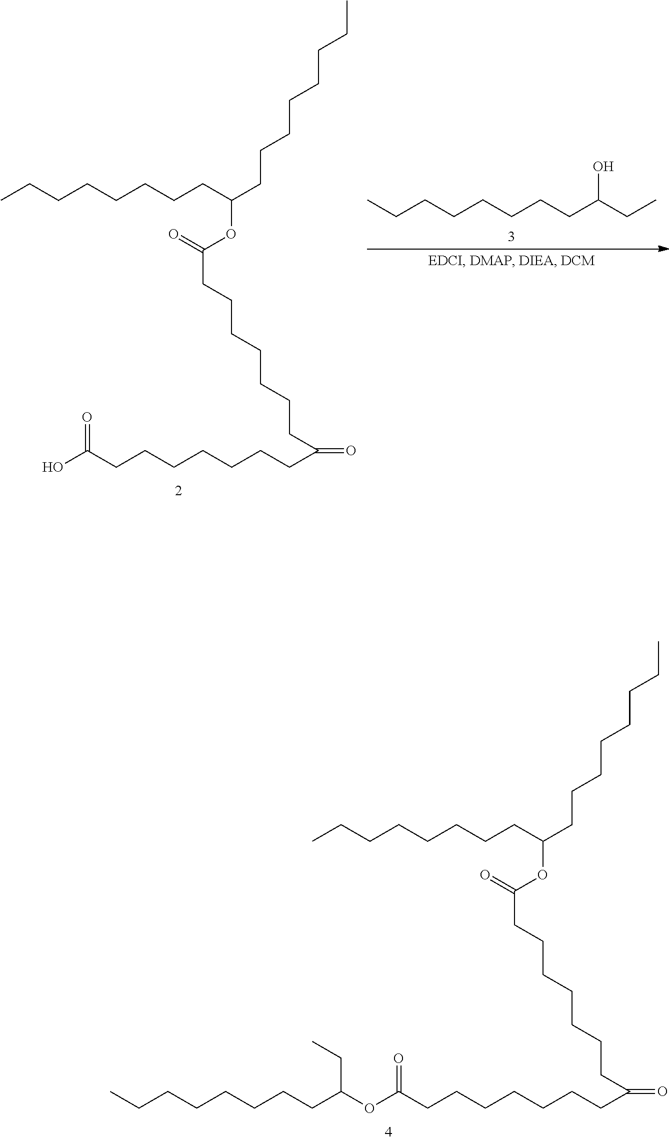 [SMILES: CCCCCCCCC(CCCCCCCC)OC(=O)CCCCCCCC(=O)CCCCCCCC(=O)O ; CCCCCCCCC(O)CC ; CCCCCCCCC(CC)OC(=O)CCCCCCCC(=O)CCCCCCCC(=O)OC(CCCCCCCC)CCCCCCCC]

To a solution of compound 2 (1.9 g, 3.44 mmol, 1 eq.), compound 3 (592.16 mg, 3.44 mmol, 1 eq.), DIEA (1.33 g, 10.31 mmol, 1.80 mL, 3 eq.) and DMAP (83.97 mg, 687.33 μmol, 0.2 eq.) in DCM (20 mL) was added EDCI (1.32 g, 6.87 mmol, 2 eq.) at 25° C. and the mixture stirred at 25° C. for 12 hours. The reaction mixture was concentrated under vacuum. The residue was purified by silica gel column chromatography (SiO₂, PE:EA=0/1˜ 20/1˜10/1) to give compound 4 (2.2 g, 86.0% yield, >95% purity) as a colorless oil.

¹HNMR: (400 MHz, CHLOROFORM-d) δ ppm 4.97-4.70 (m, 2H), 2.42-2.23 (m, 8H), 1.64-1.46 (m, 18H), 1.31-1.23 (m, 48H), 0.92-0.84 (m, 12H).

### Procedure for Preparation of Lipid 2

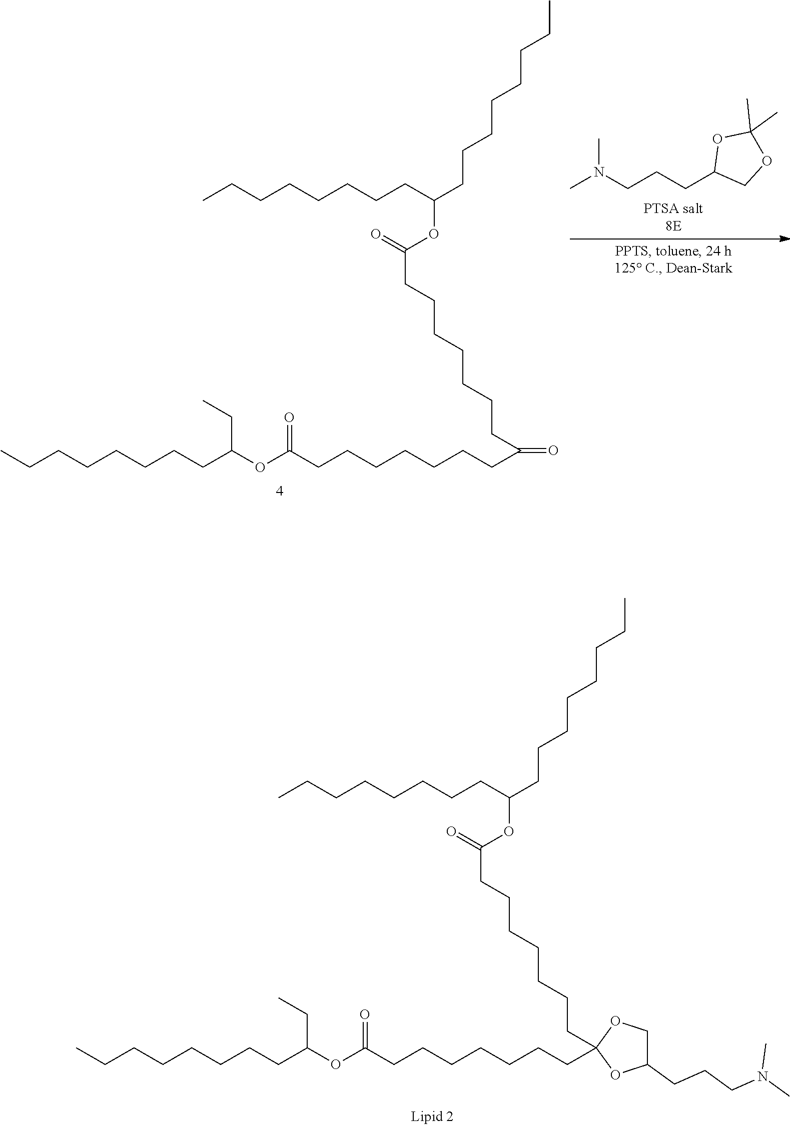

To a solution of compound 4 and compound 8E (1.02 g, 2.83 mmol, 2 eq., PTSA salt) in toluene (10 mL) was added PPTS (355.37 mg, 1.41 mmol, 1 eq.) and PTSA (53.80 mg, 282.82 μmol, 0.2 eq.) at 25° C. The mixture was stirred at 125° C. for 12 hours. The residue was poured into sat. NaHCO₃ (aqueous solution, 100 mL) and stirred for 2 mins. The aqueous phase was extracted with DCM (40 mL*3). The combined organic phases were washed with brine (50 mL), dried over anhydrous Na₂SO₄, filtered and concentrated under vacuum. The residue was purified by silica gel column chromatography (SiO₂, Petroleum ether/Ethyl acetate=10/1 to 1/1), followed by silica gel column chromatography (SiO₂, DCM/MeOH=10/1) to give Lipid 2 (472 mg, 39.51% yield, 99% purity) as a yellow oil.

LCMS: m/z 837.0 [M+1]⁺. ¹HNMR: (400 MHz, CHLOROFORM-d) δ ppm 4.97-4.79 (m, 2H), 4.16-4.01 (m, 2H), 3.56-3.42 (m, 1H), 2.46 (br s, 2H), 2.40-2.34 (m, 6H), 2.31 (td, J=3.7, 7.5 Hz, 4H), 1.69-1.51 (m, 20H), 1.40-1.24 (m, 52H), 0.99-0.85 (m, 12H).

### Synthesis of Lipid 3

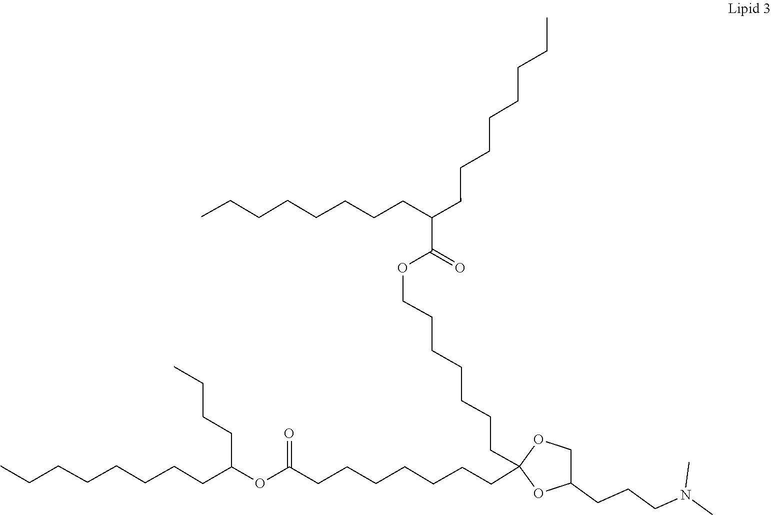 [SMILES: CCCCCCCCC(CCCC)OC(=O)CCCCCCCC1(CCCCCCCOC(=O)C(CCCCCCCC)CCCCCCCC)OCC(CCCN(C)C)O1]

A scheme for the synthesis of lipid 3 is provided in Scheme 3 below.

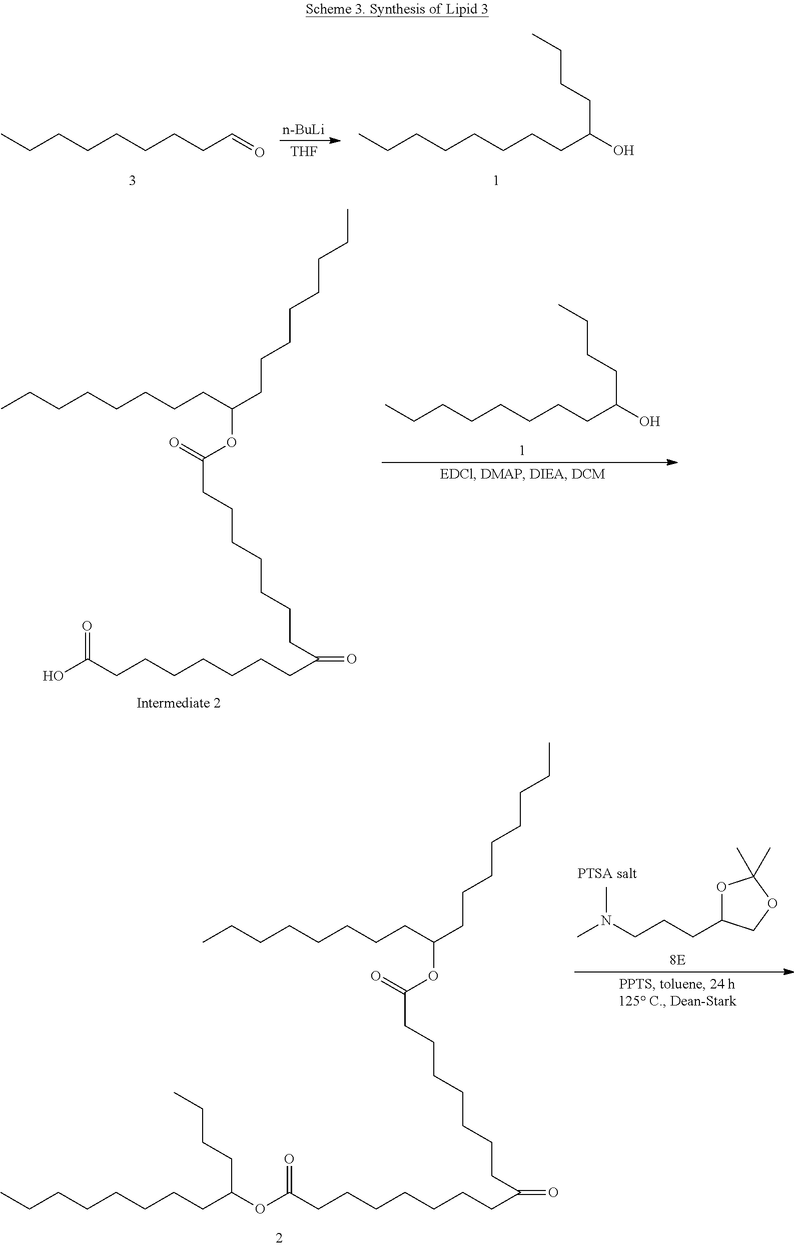

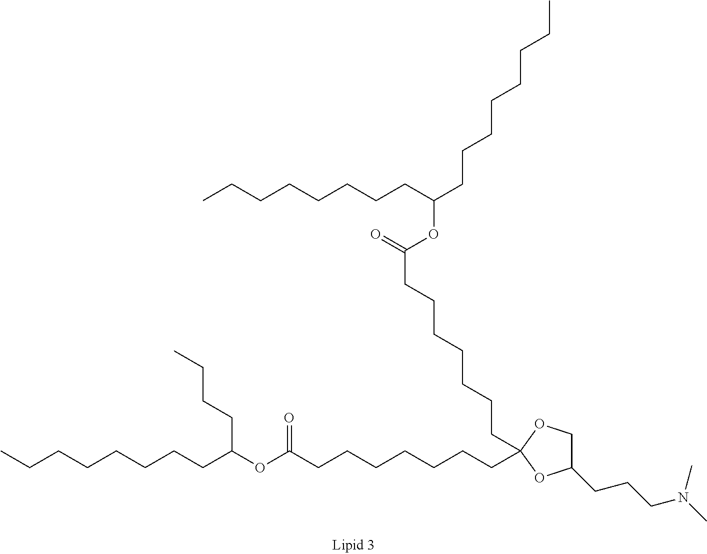 [SMILES: CCCCCCCCC(CCCC)OC(=O)CCCCCCCC1(CCCCCCCC(=O)OC(CCCCCCCC)CCCCCCCC)OCC(CCCN(C)C)O1]

### Procedure for Preparation of Compound 1

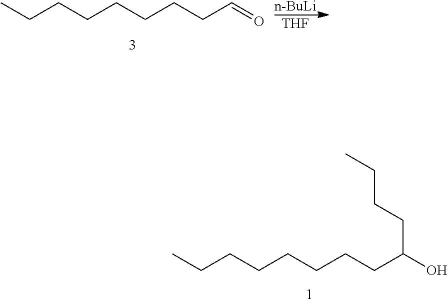

To a solution of compound 3 (1 g, 7.03 mmol, 1 eq.) in THE (10 mL) was added n-BuLi (2.5 M, 4.22 mL, 1.5 eq.) at −78° C. The mixture was stirred at −78° C. for 2 hours. Then the mixture was stirred at 25° C. for 2 hours. The mixture was poured into ice-water (w/w=1/1, 50 mL) and stirred for 2 mins. The aqueous phase was extracted with ethyl acetate (50 mL*3). The combined organic phases were washed with brine (60 mL), dried over anhydrous Na₂SO₄, filtered and concentrated under vacuum. The residue was purified by silica gel column chromatography (SiO₂, Petroleum ether/Ethyl acetate=20/1 to 10/1) to give compound 1 (1.1 g, 78.09% yield) as a colorless oil.

¹HNMR: (400 MHz, DMSO-d6) δ ppm 4.25 (br d, J=5.2 Hz, 1H), 1.45-1.19 (m, 20H), 0.90 (t, J=6.8 Hz, 6H).

### Procedure for Preparation of Compound 2

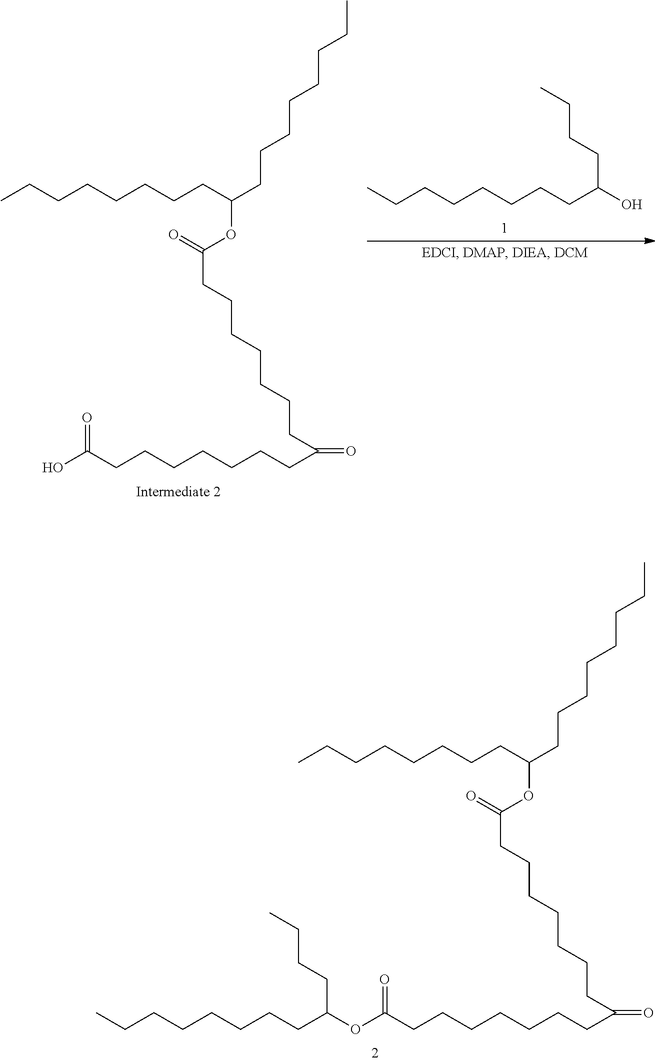 [SMILES: CCCCCCCCC(CCCCCCCC)OC(=O)CCCCCCCC(=O)CCCCCCCC(=O)O ; CCCCCCCCC(O)CCCC ; CCCCCCCCC(CCCC)OC(=O)CCCCCCCC(=O)CCCCCCCC(=O)OC(CCCCCCCC)CCCCCCCC]

To a solution of intermediate 2 (1 g, 1.81 mmol, 1 eq.) and compound 1 (543.60 mg, 2.71 mmol, 1.5 eq.) in DCM (10 mL) was added EDCI (693.48 mg, 3.62 mmol, 2 eq.), DIEA (935.08 mg, 7.24 mmol, 1.26 mL, 4 eq.) and DMAP (220.97 mg, 1.81 mmol, 1 eq.) at 25° C. The mixture was stirred at 40° C. for 12 hours. The residue was poured into ice-water (w/w=1/1, 20 mL) and stirred for 2 mins. The aqueous phase was extracted with DCM (20 mL*3). The combined organic phases were washed with brine (30 mL), dried over anhydrous Na₂SO₄, filtered and concentrated under vacuum. The residue was purified by silica gel column chromatography (SiO₂, Petroleum ether/Ethyl acetate=20/1 to 10/1) to give compound 2 (1.1 g, 82.72% yield) as a colorless oil.

¹HNMR: (400 MHz, CHLOROFORM-d) δ ppm 4.79 (t, J=6.2 Hz, 2H), 2.37-2.13 (m, 8H), 1.56-1.41 (m, 16H), 1.26-1.16 (m, 52H), 0.83-0.79 (m, 12H).

### Procedure for Preparation of Lipid 3

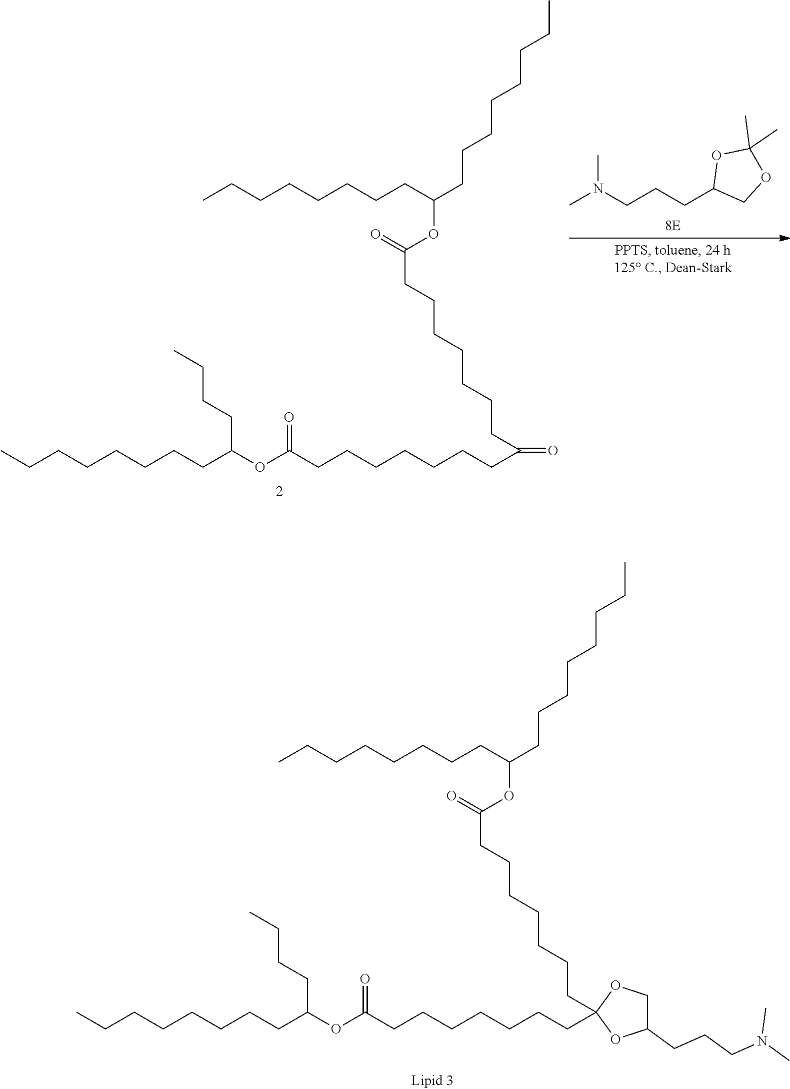

To a solution of compound 2 (1 g, 1.36 mmol, 1 eq.), PPTS (341.81 mg, 1.36 mmol, 1 eq.) and compound 8E (977.90 mg, 2.72 mmol, 3.84 eq.) in Tol. (10 mL) was added PTSA (776.17 mg, 4.08 mmol, 3 eq.) at 25° C. The mixture was stirred at 125° C. for 12 hours equipping with Dean-Stark. The reaction mixture was poured into sat. NaHCO3 (aqueous solution, 100 mL) and stirred for 2 mins. The aqueous phase was extracted with DCM (40 mL*3). The combined organic phases were washed with brine (50 mL), dried over anhydrous Na₂SO₄, filtered and concentrated under vacuum. The residue was purified by silica gel column chromatography (SiO₂, DCM/MeOH=20/1 to 10/1) to give Lipid 3 (140 mg, 11.79% yield, 99% purity) as a yellow oil.

LCMS: m/z 865.1 [M+1]⁺. ¹HNMR: (400 MHz, CHLOROFORM-d) δ ppm 4.87 (t, J=6.0 Hz, 2H), 4.15-3.95 (m, 2H), 3.53-3.40 m, 1H), 2.46-2.19 (m, 12H), 1.70-1.46 (m, 22H), 1.39-1.19 (m, 56H), 0.98-0.81 (m, 12H).

### Synthesis of Lipid 4

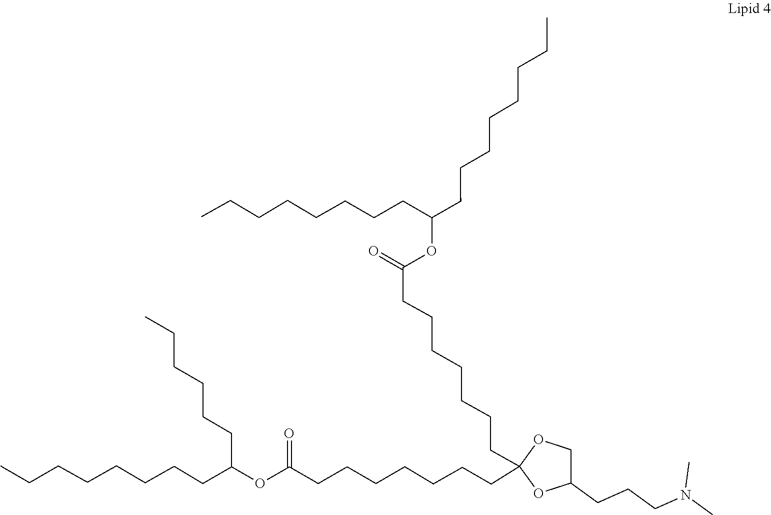 [SMILES: CCCCCCCCC(CCCCCC)OC(=O)CCCCCCCC1(CCCCCCCC(=O)OC(CCCCCCCC)CCCCCCCC)OCC(CCCN(C)C)O1]

A scheme for the synthesis of Lipid 4 is provided in Scheme 4 below.

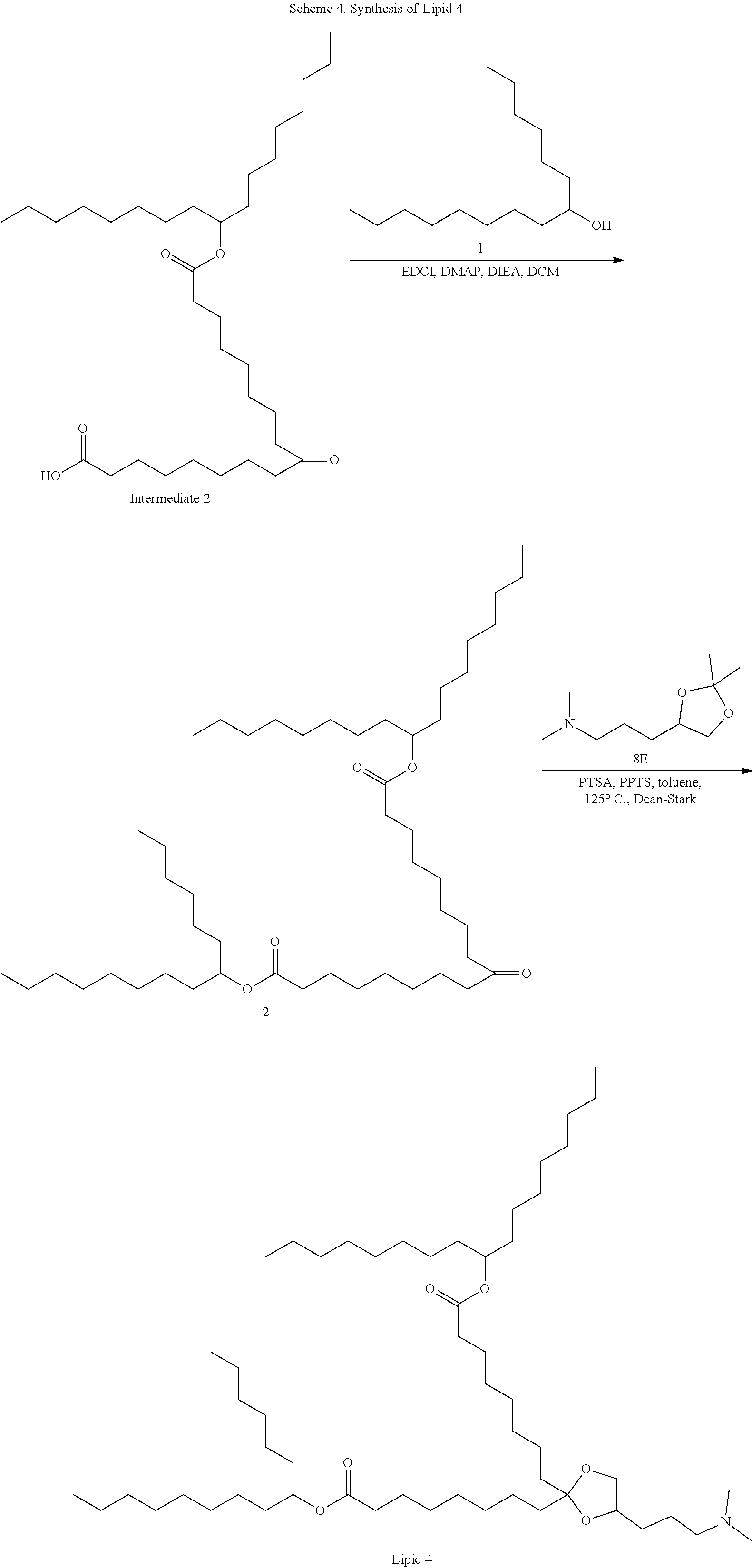

### Procedure for Preparation of Compound 2

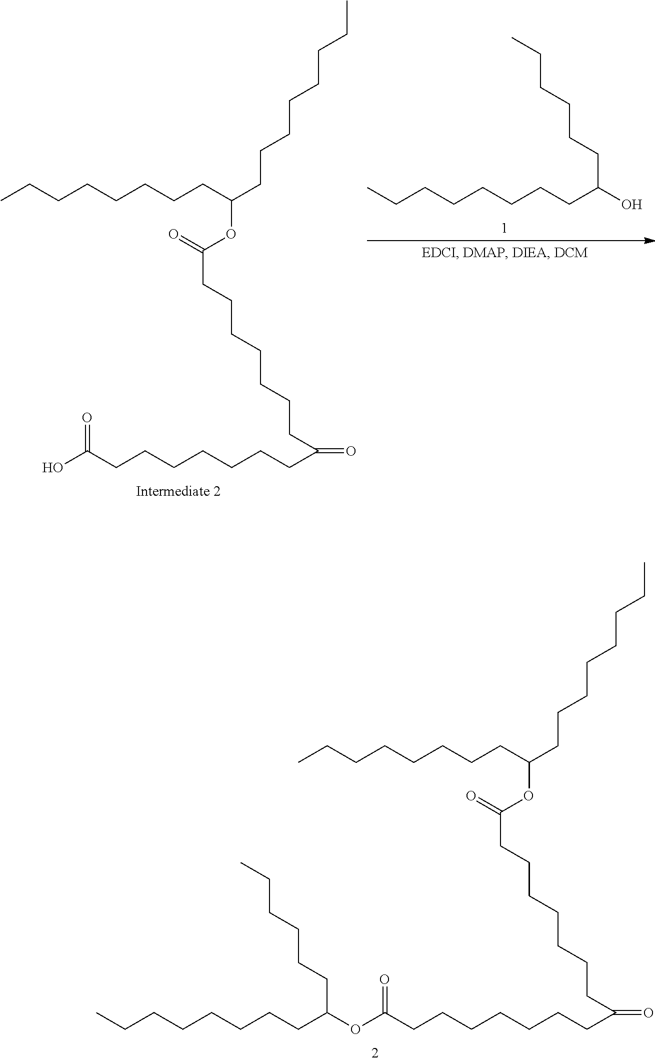 [SMILES: CCCCCCCCC(CCCCCCCC)OC(=O)CCCCCCCC(=O)CCCCCCCC(=O)O ; CCCCCCCCC(O)CCCCCC ; CCCCCCCCC(CCCCCC)OC(=O)CCCCCCCC(=O)CCCCCCCC(=O)OC(CCCCCCCC)CCCCCCCC]

To a solution of intermediate 2 (1.5 g, 2.71 mmol, 1 eq.) and compound 1 (619.71 mg, 2.71 mmol, 1 eq.) in DCM (20 mL) was added DIEA (1.40 g, 10.85 mmol, 1.89 mL, 4 eq.), DMAP (331.46 mg, 2.71 mmol, 1 eq.) and EDCI (1.04 g, 5.43 mmol, 2 eq) at 25° C. The mixture was stirred at 40° C. for 12 hours. The reaction mixture was poured into ice-water (w/w=1/1, 10 mL) and stirred for 2 mins. The aqueous phase was extracted with DCM (20 mL*3). The combined organic phases were washed with brine (30 mL), dried over anhydrous Na₂SO₄, filtered and concentrated under vacuum. The residue was purified by silica gel column chromatography (SiO₂, Petroleum ether/Ethyl acetate=20/1 to 10/1) to give compound 2 (1.7 g, 82.09% yield) as a colorless oil.

¹HNMR: (400 MHz, CHLOROFORM-d) δ ppm 4.79 (t, J=6.0 Hz, 2H), 2.41-2.09 (m, 8H), 1.56-1.41 (m, 16H), 1.31-1.10 (m, 56H), 0.81 (t, J=6.8 Hz, 12H).

### Procedure for Preparation of Lipid 4

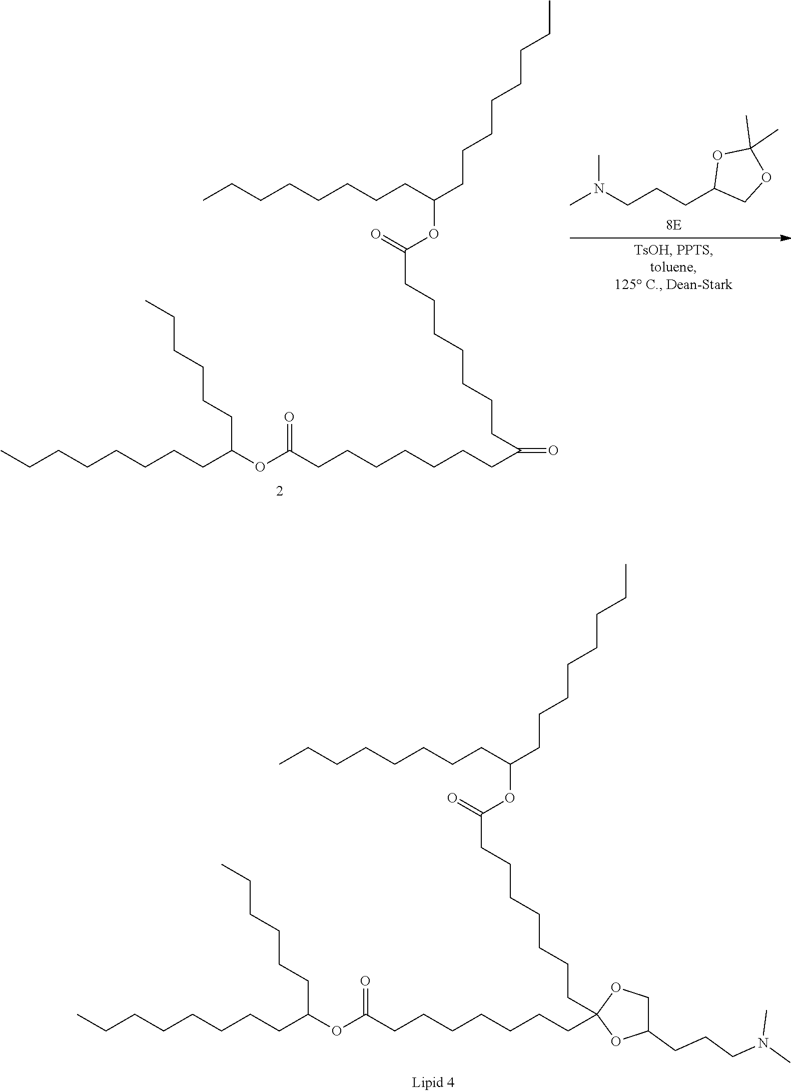

To a solution of compound 2 (1.7 g, 2.23 mmol, 1 eq.) and compound 8E (1.25 g, 6.68 mmol, 3 eq., free base) in toluene (30 mL) was added PTSA (1.34 g, 7.80 mmol, 3.5 eq.) and PPTS (559.72 mg, 2.23 mmol, 1 eq.) at 25° C. The mixture was stirred at 125° C. for 12 hours equipping with a Dean-Stark. The reaction mixture was poured into sat. NaHCO₃ (aqueous solution, 100 mL) and stirred for 2 mins. The aqueous phase was extracted with DCM (40 mL*3). The combined organic phases were washed with brine (50 mL), dried over anhydrous Na₂SO₄, filtered and concentrated under vacuum. The residue was purified by silica gel column chromatography (SiO₂, DCM/MeOH=20/1 to 10/1) to give Lipid 4 (320 mg, 15.94% yield, 99% purity) as a yellow oil.

LCMS: m/z 893.1 [M+1]⁺. ¹HNMR: (400 MHz, CHLOROFORM-d) δ ppm 4.86 (t, J=6.0 Hz, 2H), 4.12-3.97 (m, 2H), 3.51-3.39 (m, 1H), 2.33-2.24 (m, 6H), 2.22 (s, 6H), 1.67-1.42 (m, 20H), 1.41-1.13 (m, 60H), 0.87 (t, J=6.8 Hz, 12H).

### Synthesis of Lipid 5

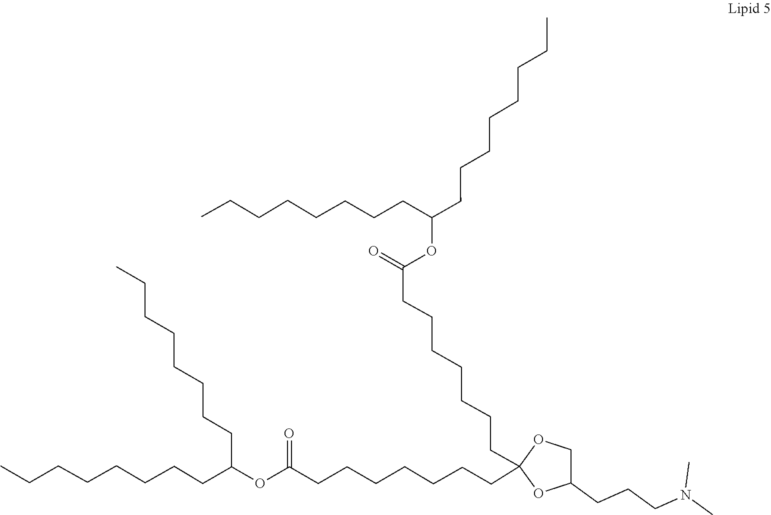 [SMILES: CCCCCCCCC(CCCCCCCC)OC(=O)CCCCCCCC1(CCCCCCCC(=O)OC(CCCCCCCC)CCCCCCCC)OCC(CCCN(C)C)O1]

A scheme for the synthesis of Lipid 5 is provided in Scheme 5 below.

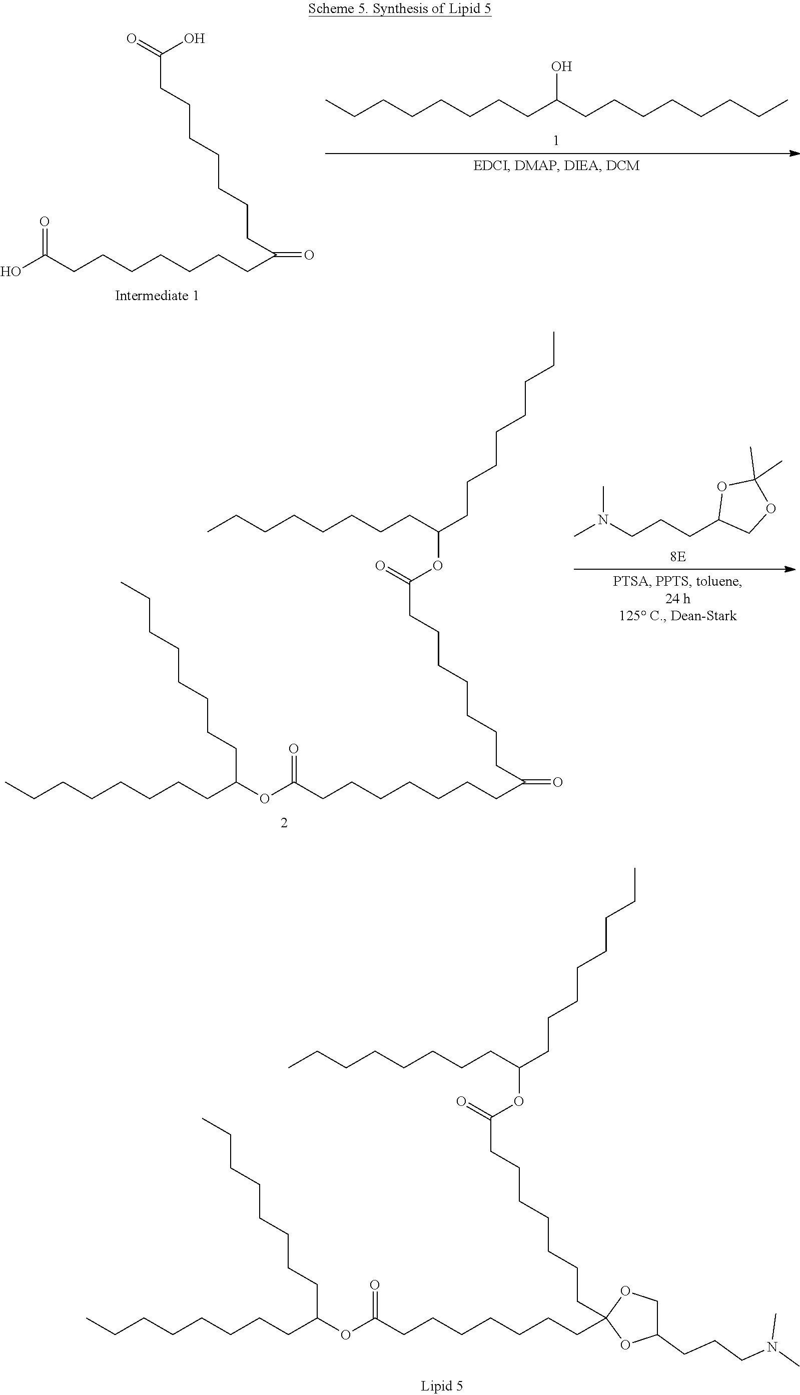

### Procedure for Preparation of Compound 2

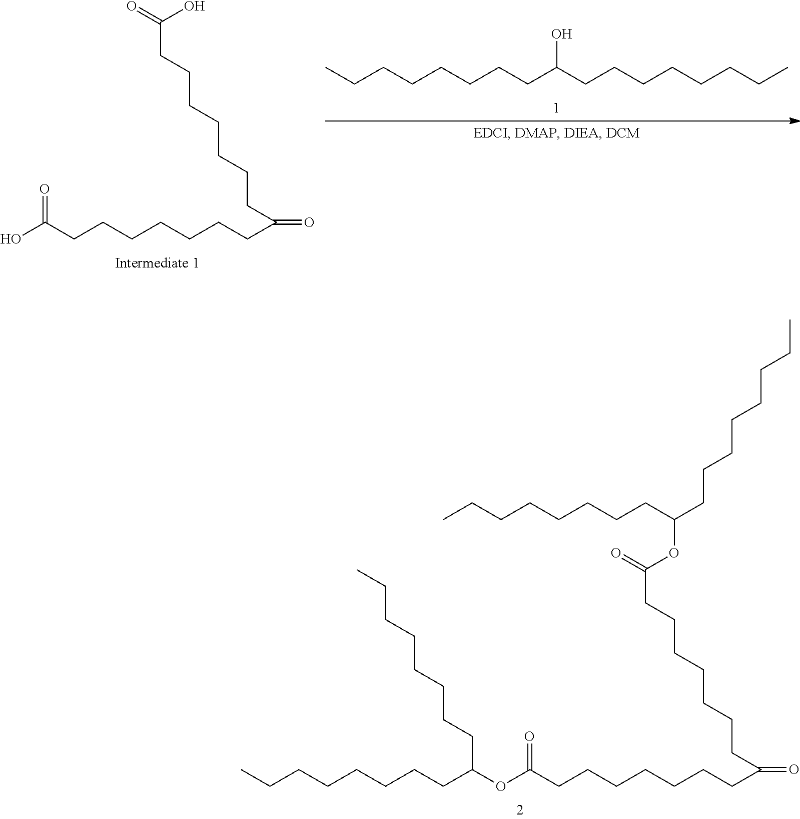 [SMILES: O=C(O)CCCCCCCC(=O)CCCCCCCC(=O)O ; CCCCCCCCC(O)CCCCCCCC ; CCCCCCCCC(CCCCCCCC)OC(=O)CCCCCCCC(=O)CCCCCCCC(=O)OC(CCCCCCCC)CCCCCCCC]

To a solution of intermediate 1 (2 g, 6.36 mmol, 1 eq.) and compound 1 (3.92 g, 15.27 mmol, 2.4 eq.) in DCM (20 mL) was added EDCI (4.88 g, 25.44 mmol, 4 eq.), DMAP (777.10 mg, 6.36 mmol, 1 eq.) and DIEA (6.58 g, 50.89 mmol, 8.86 mL, 8 eq.) at 25° C. The mixture was stirred at 40° C. for 12 hours. The reaction mixture was poured into ice-water (w/w=1/1, 40 mL) and stirred for 2 mins. The aqueous phase was extracted with DCM (40 mL*3). The combined organic phases were washed with brine (50 mL), dried over anhydrous Na₂SO₄, filtered and concentrated under vacuum. The residue was purified by silica gel column chromatography (SiO₂, Petroleum ether/Ethyl acetate=30/1 to 20/1) to give compound 2 (2.7 g, 53.64% yield) as a colorless oil.

¹HNMR: (400 MHz, CHLOROFORM-d) δ ppm 4.90 (t, J=6.2 Hz, 2H), 2.41 (t, J=7.5 Hz, 4H), 2.31 (t, J=7.6 Hz, 4H), 1.67-1.51 (m, 18H), 1.38-1.24 (m, 58H), 0.91 (t, J=6.8 Hz, 12H)

### Procedure for Preparation of Lipid 5

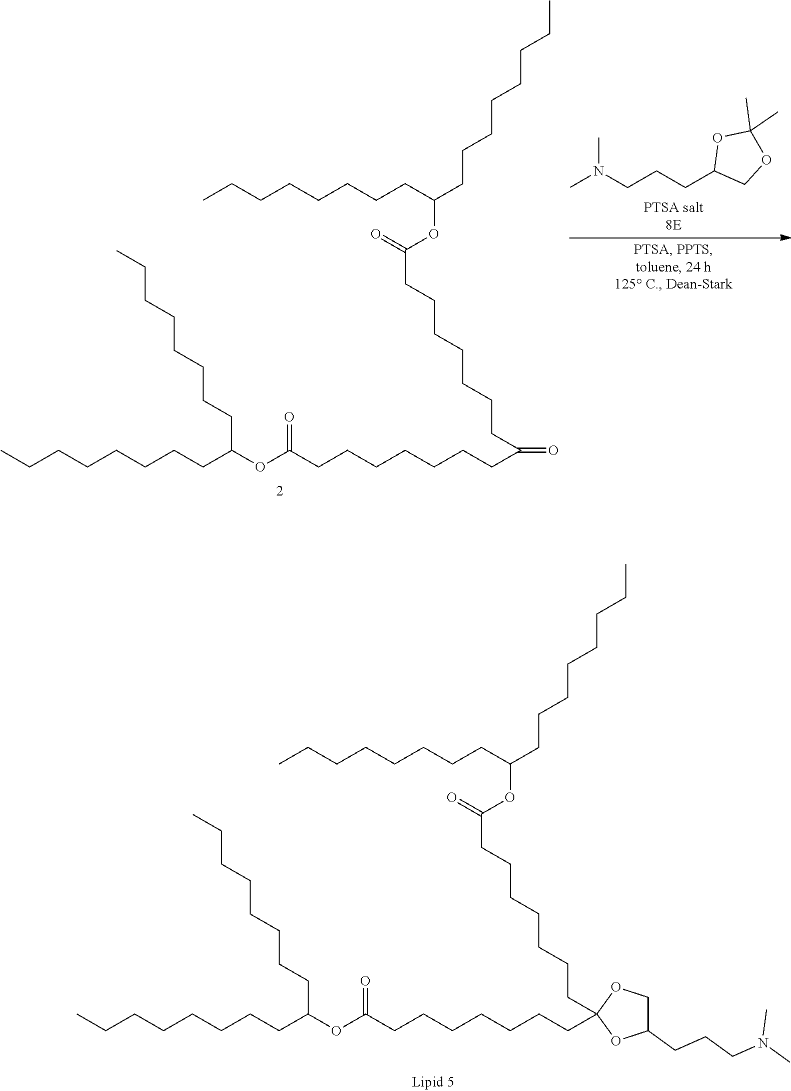

To a solution of compound 2 (1 g, 1.26 mmol, 1 eq.) and compound 8E (1.36 g, 3.79 mmol, 3 eq., PTSA salt) in toluene (15 mL) was added PTSA (120.19 mg, 631.86 μmol, 0.5 eq.) and PPTS (317.57 mg, 1.26 mmol, 1 eq.) at 25° C. The mixture was stirred at 125° C. for 12 hours equipping with a Dean-Stark. The reaction mixture was poured into sat. NaHCO₃ (aqueous solution, 100 mL) and stirred for 2 mins. The aqueous phase was extracted with DCM (40 mL*3). The combined organic phases were washed with brine (50 mL), dried over anhydrous Na₂SO₄, filtered and concentrated under vacuum. The residue was purified by silica gel column chromatography (SiO₂, DCM/MeOH=20/1 to 10/1) to give Lipid 5 (450 mg, 38.30% yield, 99% purity) as a yellow oil.

LCMS: m/z 921.1 [M+1]⁺. HNMR: (400 MHz, CHLOROFORM-d) δ ppm 4.79 (quin, J=6.2 Hz, 2H), 4.05-3.89 (m, 2H), 3.45-3.30 (m, 1H), 2.20 (t, J=7.5 Hz, 6H), 2.15 (s, 6H), 1.52-1.38 (m, 20H), 1.30-1.13 (m, 64H), 0.85-0.77 (m, 12H).

### Synthesis of Lipid 6

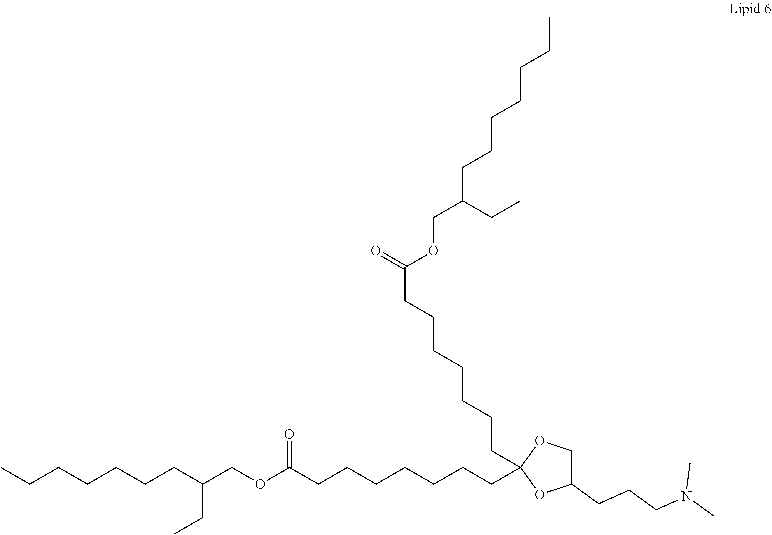 [SMILES: CCCCCCCC(CC)COC(=O)CCCCCCCC1(CCCCCCCC(=O)OCC(CC)CCCCCCC)OCC(CCCN(C)C)O1]

A scheme for the synthesis of Lipid 6 is provided in Scheme 6 below.

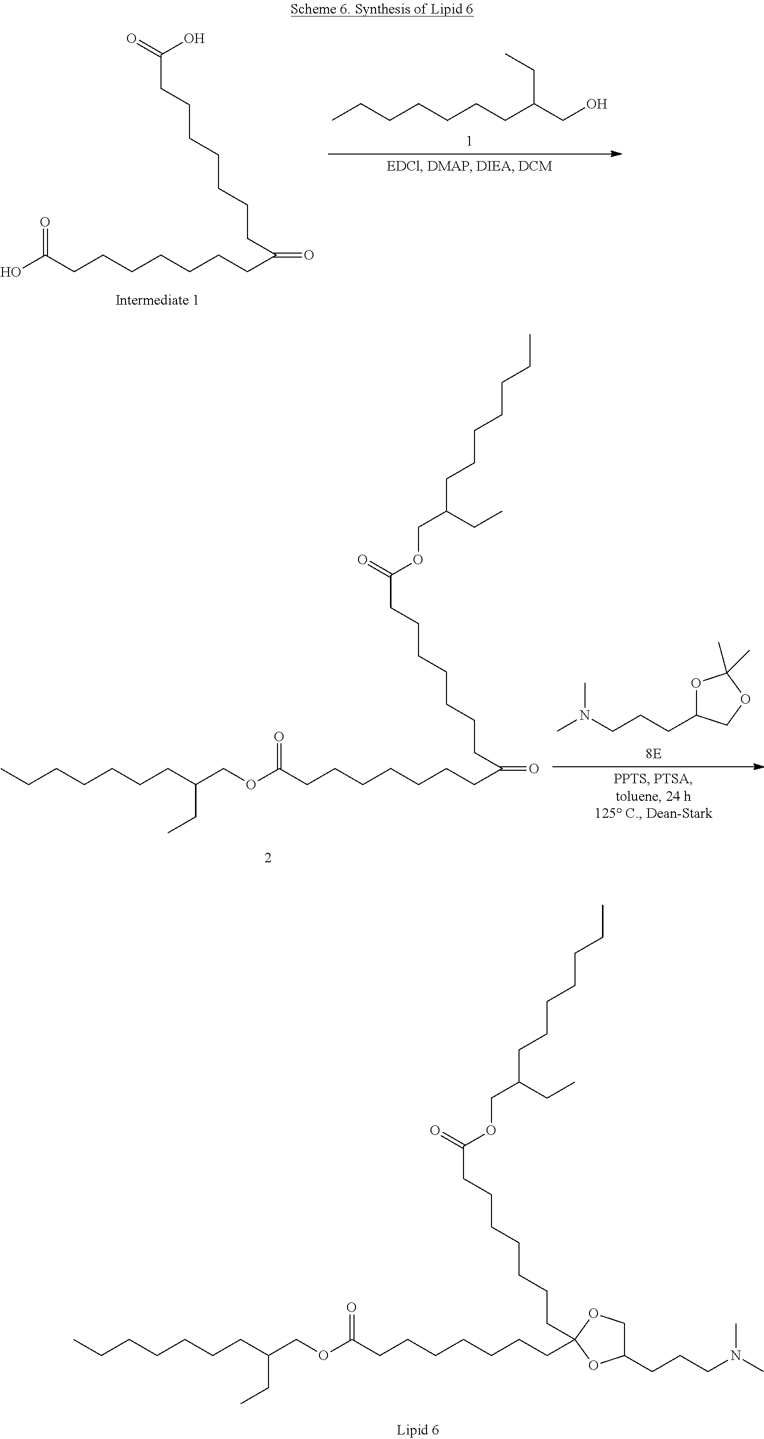

### Procedure for Preparation of Compound 2

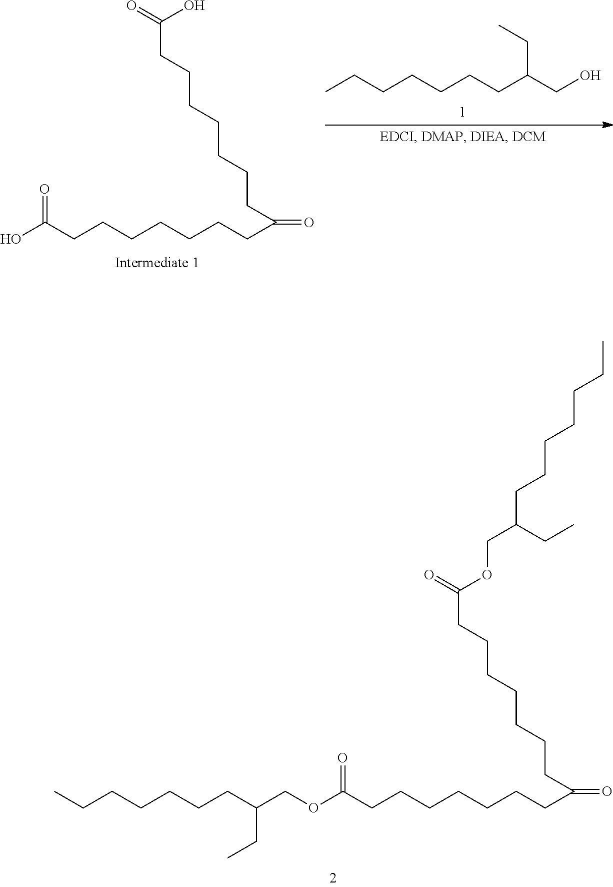 [SMILES: O=C(O)CCCCCCCC(=O)CCCCCCCC(=O)O ; CCCCCCCC(CC)CO ; CCCCCCCC(CC)COC(=O)CCCCCCCC(=O)CCCCCCCC(=O)OCC(CC)CCCCCCC]

To a solution of intermediate 1 (800 mg, 2.54 mmol, 1 eq.) and compound 1 (876.83 mg, 5.09 mmol, 2 eq.) in DCM (10 mL) was added EDCI (1.95 g, 10.18 mmol, 4 eq.), DIEA (2.63 g, 20.36 mmol, 3.55 mL, 8 eq.) and DMAP (155.42 mg, 1.27 mmol, 0.5 eq.) at 25° C. The mixture was stirred at 40° C. for 12 hours. The reaction mixture was poured into ice-water (w/w=1/1, 40 mL) and stirred for 2 mins. The aqueous phase was extracted with DCM (40 mL*3). The combined organic phases were washed with brine (50 mL), dried over anhydrous Na₂SO₄, filtered and concentrated under vacuum. The residue was purified by silica gel column chromatography (SiO₂, Petroleum ether/Ethyl acetate=30/1 to 20/1) to give compound 2 (1.1 g, 69.39% yield) as a colorless oil.

¹HNMR: (400 MHz, CHLOROFORM-d) δ ppm 4.02 (br d, J=5.6 Hz, 4H), 2.49-2.27 (m, 8H), 1.67-1.58 (m, 16H), 1.38-1.27 (m, 36H), 0.93 (br t, J=7.2 Hz, 12H).

### Procedure for Preparation of Lipid 6

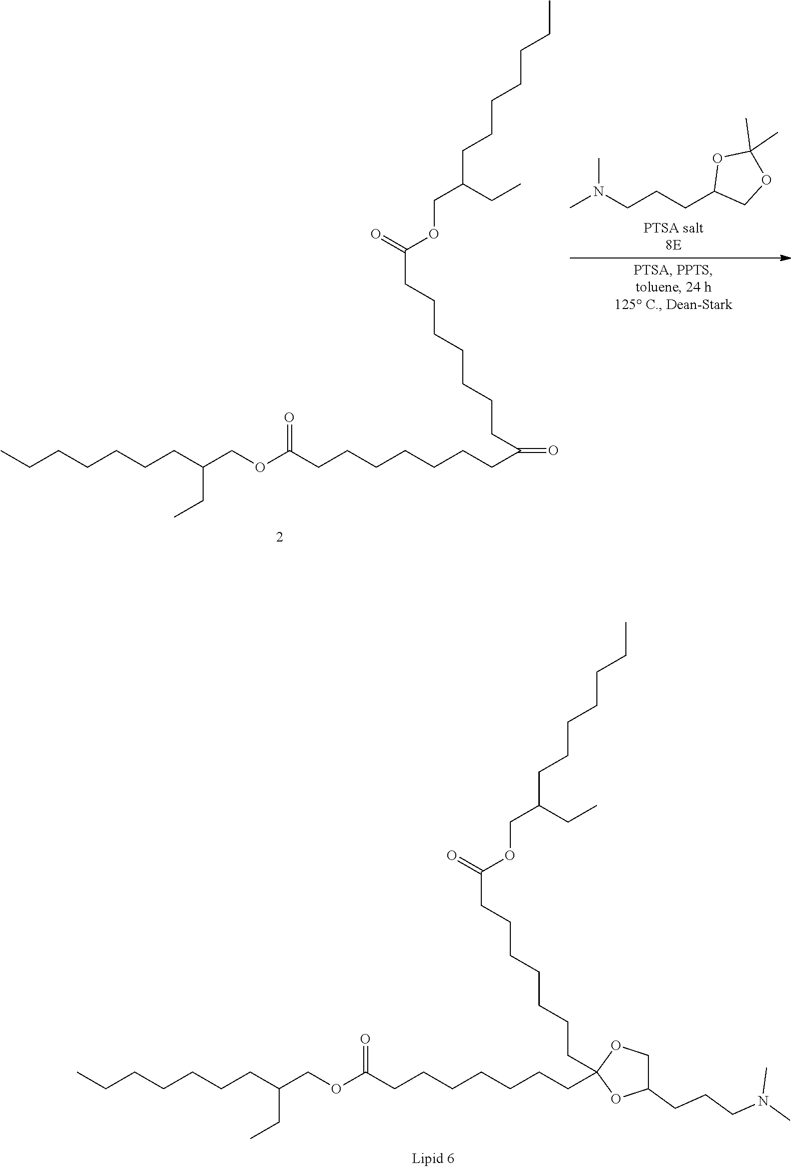

To a solution of compound 2 (1 g, 1.61 mmol, 1 eq.) and compound 8E (1.73 g, 4.82 mmol, 3 eq., PTSA salt) in toluene (10 mL) was added PPTS (403.37 mg, 1.61 mmol, 1 eq.) and PTSA (152.66 mg, 802.57 μmol, 0.5 eq.) at 25° C. The mixture was stirred at 125° C. for 12 hours equipping with a Dean-Stark. The reaction mixture was poured into sat. NaHCO₃ (aqueous solution, 100 mL) and stirred for 2 mins. The aqueous phase was extracted with DCM (40 mL*3). The combined organic phases were washed with brine (50 mL), dried over anhydrous Na₂SO₄, filtered and concentrated under vacuum. The residue was purified by silica gel column chromatography (SiO₂, DCM/MeOH=20/1 to 10/1) to give Lipid 6 (170 mg, 13.94% yield, 99% purity) as a yellow oil.

LCMS: m/z 752.9 [M+1]⁺. ¹HNMR: (400 MHz, CHLOROFORM-d) δ ppm 4.00-3.91 (m, 2H), 3.88 (dd, J=1.6, 5.6 Hz, 4H), 3.35 (s, 1H), 2.20 (t, J=7.6 Hz, 6H), 2.13 (s, 6H), 1.58-1.41 (m, 16H), 1.26-1.13 (m, 42H), 0.84-0.74 (m, 12H).

### Synthesis of Lipid 7

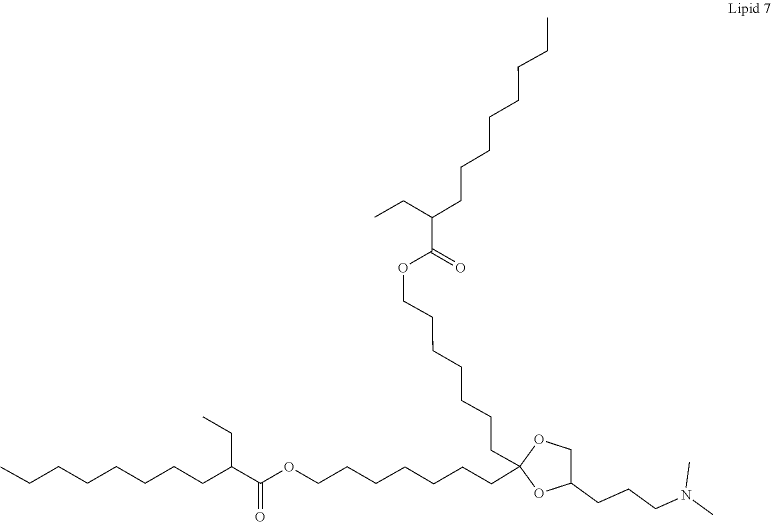 [SMILES: CCCCCCCCC(CC)C(=O)OCCCCCCCC1(CCCCCCCOC(=O)C(CC)CCCCCCCC)OCC(CCCN(C)C)O1]

A scheme for the synthesis of Lipid 7 is provided in Scheme 7 below.

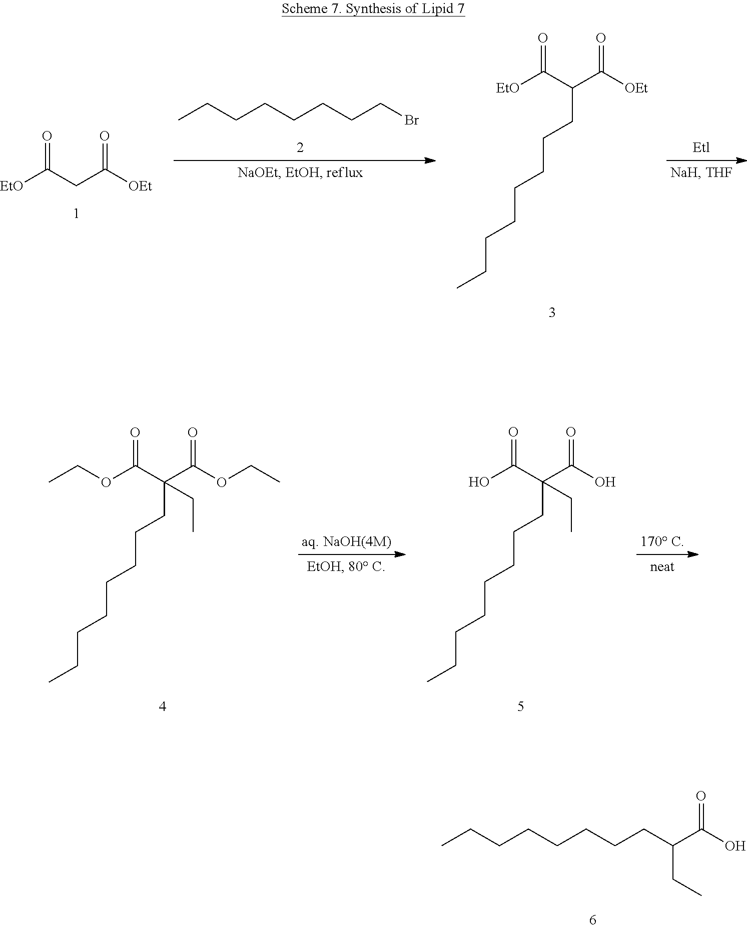

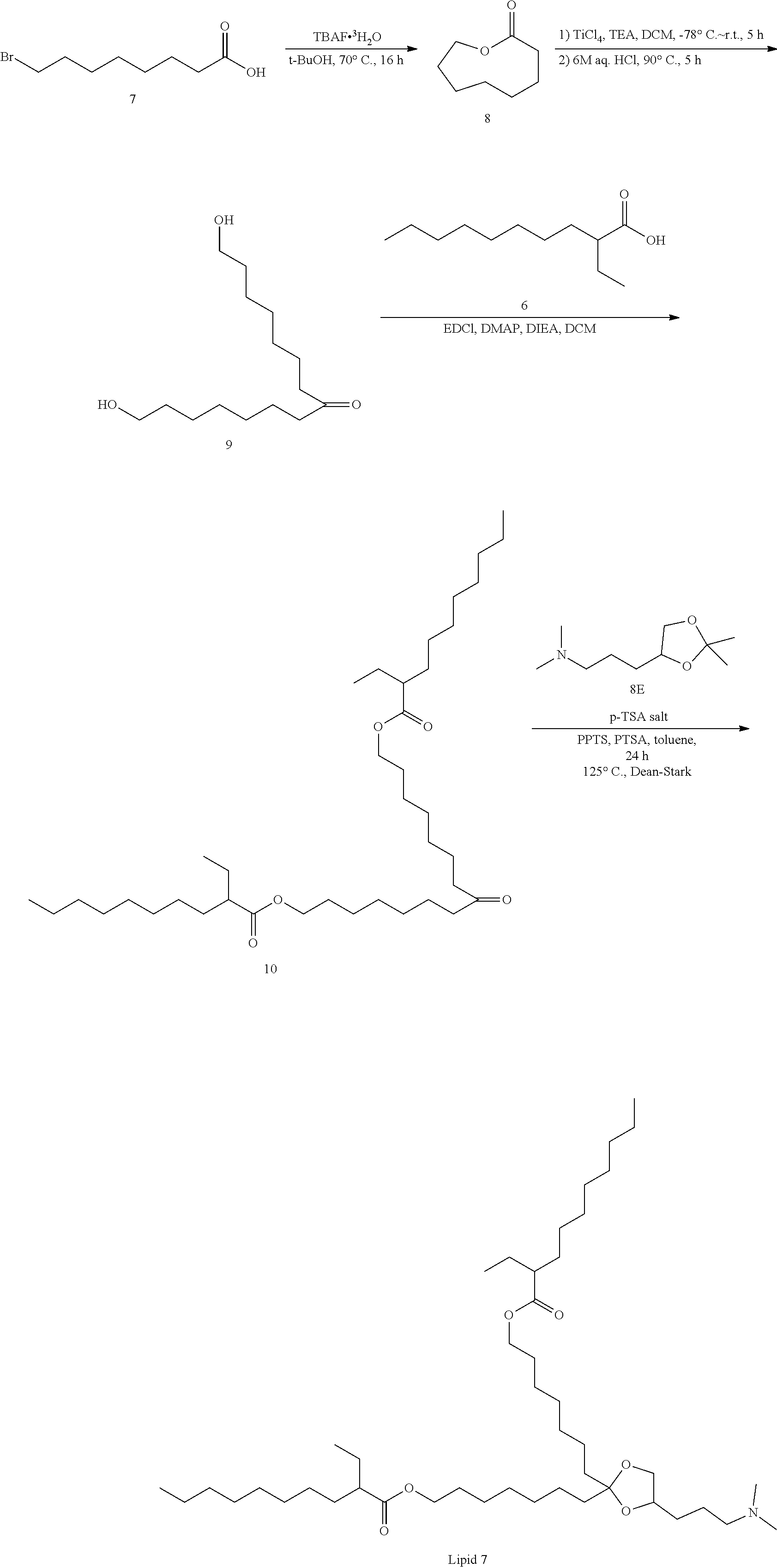

### Procedure for Preparation of Compound 3

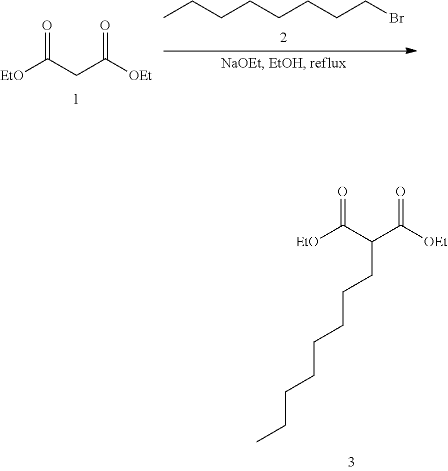

To a solution of compound 1 (5 g, 31.22 mmol, 4.74 mL, 1 eq.) in EtOH (50 mL) was added NaOEt (10.62 g, 31.22 mmol, 20% EtOH solution, 1 eq.) dropwise at 0° C. Then 1-bromooctane (6.03 g, 31.22 mmol, 5.43 mL, 1 eq.) was added at 25° C. The mixture was stirred at 60° C. for 12 hours. The reaction mixture was directly concentrated under reduced pressure to give the crude product. The residue was purified by column chromatography (SiO₂, Petroleum ether/Ethyl acetate=20/1 to 10/1) to give compound 3 (6.8 g, 79.97% yield) as a light yellow oil.

¹HNMR: (400 MHz, CHLOROFORM-d) δ ppm 4.22-4.03 (m, 4H), 3.30-3.18 (m, 1H), 1.81 (q, J=7.4 Hz, 2H), 1.38-1.09 (m, 18H), 0.86-0.76 (m, 3H)

### Procedure for Preparation of Compound 4

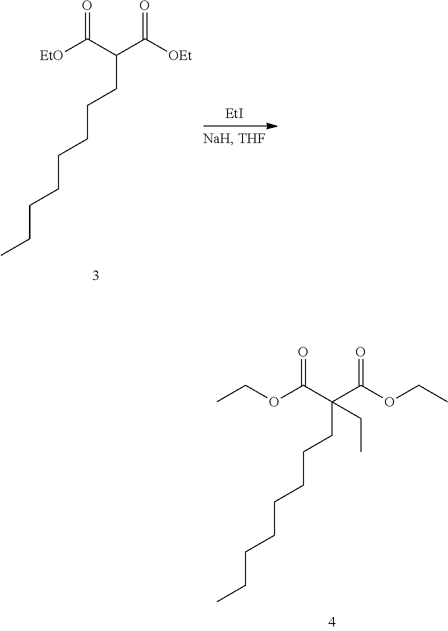 [SMILES: "EtI" ; CCCCCCCCC(C(=O)OCC)C(=O)OCC ; CCCCCCCCC(CC)(C(=O)OCC)C(=O)OCC]

To a solution of compound 3 (3 g, 11.01 mmol, 1 eq.) in THF (30 mL) was added NaH (660.78 mg, 16.52 mmol, 60% purity, 1.5 eq.) at 0° C. The mixture was stirred at 0° C. for 0.5 hour. Then EtI (2.58 g, 16.52 mmol, 1.32 mL, 1.5 eq.) was added dropwise. The mixture was stirred at 25° C. for 2 hours. The reaction mixture was quenched by addition of H₂O (50 mL) at 0° C., and then extracted with EtOAc (50 mL*3). The combined organic layers were washed with brine (60 mL), dried over anhydrous Na₂SO₄, filtered and concentrated under reduced pressure to give a residue. The residue was purified by column chromatography (SiO₂, Petroleum ether/Ethyl acetate=20/1 to 10/1) to give compound 4 (3 g, 90.66% yield) as a colorless oil.

¹HNMR: (400 MHz, DMSO-d6) δ ppm 12.71-11.10 (m, 2H), 2.38 (t, J=7.2 Hz, 4H), 2.24-2.13 (m, 4H), 1.54-1.36 (m, 8H), 1.30-1.15 (m, 12H)

### Procedure for Preparation of Compound 5

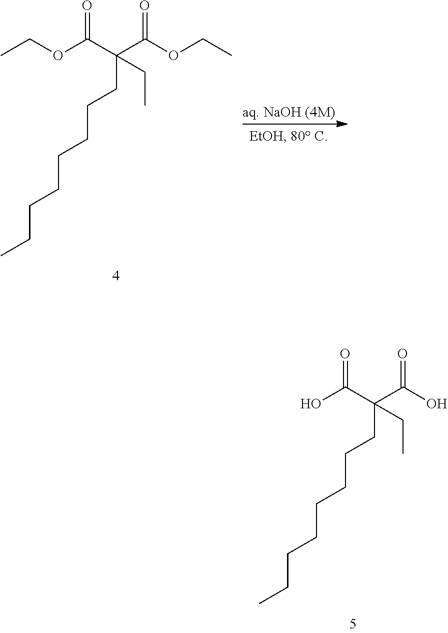 [SMILES: CCCCCCCCC(CC)(C(=O)OCC)C(=O)OCC ; "aq. NaOH (4M)" ; CCCCCCCCC(CC)(C(=O)O)C(=O)O]

To a solution of compound 4 (2.9 g, 9.65 mmol, 1 eq.) in EtOH (20 mL) was added NaOH (4 M aqueous solution, 33.78 mL, 7 eq.) at 25° C. The mixture was stirred at 80° C. for 12 hours. The reaction mixture was quenched by addition of HCl (conc., 30 mL) at 0° C., and then extracted with EtOAc (30 mL*3). The combined organic layers were washed with brine (40 ml), dried over anhydrous Na₂SO₄, filtered and concentrated under reduced pressure to give compound 5 (2.3 g, crude) as a yellow solid, which was used directly for next step without further purification.

### Procedure for Preparation of Compound 6

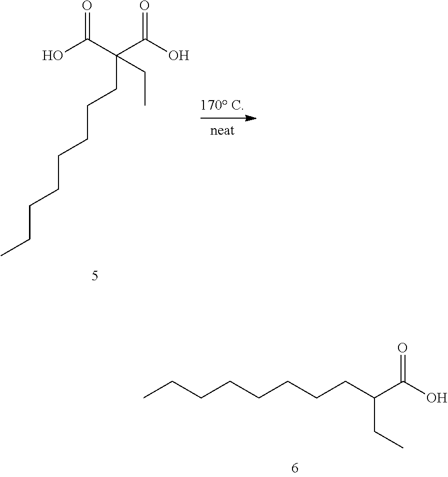 [SMILES: CCCCCCCCC(CC)(C(=O)O)C(=O)O ; CCCCCCCCC(CC)C(=O)O]

The compound 5 (5.2 g, 21.28 mmol, 1 eq.) in a 50 ml flask without solvent was heated to 170° C. for 5 hours. The residue was directly purified by column chromatography (SiO₂, Petroleum ether/Ethyl acetate=10/1 to 5/1) to give compound 6 (3.6 g, 84.44% yield) as a light-yellow oil.

¹HNMR: (400 MHz, CHLOROFORM-d) δ ppm 2.26 (tt, J=5.4, 8.6 Hz, 1H), 1.70-1.39 (m, 4H), 1.36-1.14 (m, 12H), 0.96-0.80 (m, 6H).

### Procedure for Preparation of Compound 8

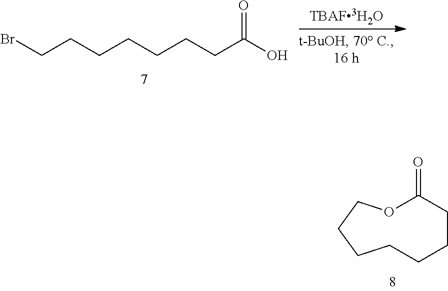 [SMILES: O=C(O)CCCCCCCBr ; O=C1CCCCCCCO1]

To a solution of compound 7 (10 g, 44.82 mmol, 1 eq.) in 1-BuOH (100 mL) was added TBAF 0.3H₂O (28.28 g, 89.64 mmol, 2 eq.) at 25° C. The mixture was stirred at 70° C. for 12 hours. The reaction mixture was concentrated under reduced pressure to give the crude product. The residue was purified by column chromatography (SiO₂, Petroleum ether/Ethyl acetate=20/1 to 10/1) to give compound 8 (3.4 g, 53.35% yield) as a white solid.

¹HNMR: (400 MHz, CHLOROFORM-d) δ ppm 4.09-3.98 (m, 2H), 2.31-2.19 (m, 2H), 1.65-1.48 (m, 4H), 1.37-1.22 (m, 6H).

### Procedure for Preparation of Compound 9

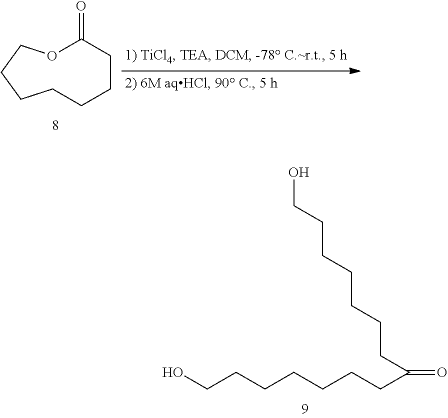 [SMILES: O=C1CCCCCCCO1 ; O=C(CCCCCCCO)CCCCCCCO]

To a solution of compound 8 in DCM (40 mL) was added TEA (3.20 g, 31.65 mmol, 4.40 mL, 1.5 eq.) and TiCl₄ (5.20 g, 27.43 mmol, 1.3 eq.) at −78° C. The mixture was stirred at 25° C. for 4 hours. The residue was poured into ice-water (w/w=1/1, 40 mL) and stirred for 2 mins. The aqueous phase was extracted with a mixed solvents of DCM:MeOH=10:1 (30 mL*3). The combined organic phases were washed with brine (3 mL), dried over anhydrous Na₂SO₄, filtered and concentrated under vacuum. To the residue was added HCl (6 M aqueous solution, 20 mL, 5.69 eq.), and then the mixture was stirred at 80° C. for 4 hours. The mixture was poured into ice-water (w/w=1/1, 50 mL) and stirred for 2 mins. The aqueous phase was extracted with a mixed solvents of DCM:MeOH=10:1 (100 mL*3). The combined organic phases were washed with brine (100 mL), dried over anhydrous Na₂SO₄, filtered and concentrated under vacuum. The residue was purified by column chromatography (SiO₂, PE/EA=5/1 to 0/1) to give compound 9 (3.5 g, 64.20% yield) as a yellow solid.

¹HNMR: (400 MHz, DMSO-d6) δ ppm 4.31 (br s, 1H), 3.99 (br t, J=6.4 Hz, 1H), 3.37 (t, J=6.4 Hz, 3H), 2.38 (br t, J=7.2 Hz, 3H), 2.27 (br t, J=7.2 Hz, 1H), 1.60-1.35 (m, 8H), 1.25 (br d, J=7.2 Hz, 12H)

### Procedure for Preparation of Compound 10

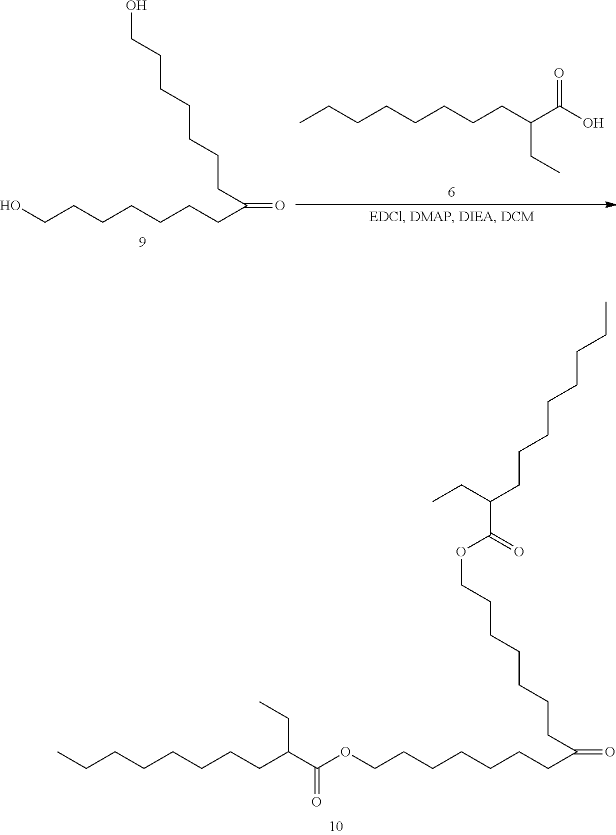 [SMILES: O=C(CCCCCCCO)CCCCCCCO ; CCCCCCCCC(CC)C(=O)O ; CCCCCCCCC(CC)C(=O)OCCCCCCCC(=O)CCCCCCCOC(=O)C(CC)CCCCCCCC]

To a solution of compound 9 (1 g, 3.87 mmol, 1 eq.) and compound 6 (1.55 g, 7.74 mmol, 2 eq.) in DCM (10 mL) was added EDCI (2.97 g, 15.48 mmol, 4 eq.), DIEA (4.50 g, 34.83 mmol, 6.07 mL, 9 eq.) and DMAP (472.79 mg, 3.87 mmol, 1 eq.) at 25° C. The mixture was stirred at 40° C. for 12 hours. The reaction mixture was concentrated under reduced pressure to give the crude product. The residue was purified by column chromatography (SiO₂, Petroleum ether/Ethyl acetate=20/1 to 10/1) to give compound 10 (920 mg, 38.16% yield) as a light yellow oil.

¹HNMR: (400 MHz, CHLOROFORM-d) δ ppm 4.07 (t, J=6.4 Hz, 4H), 2.39 (t, J=7.2 Hz, 4H), 2.32-2.21 (m, 2H), 1.68-1.58 (m, 10H), 1.56-1.40 (m, 6H), 1.39-1.20 (m, 36H), 1.00-0.80 (m, 12H)

### Procedure for Preparation of Lipid 7

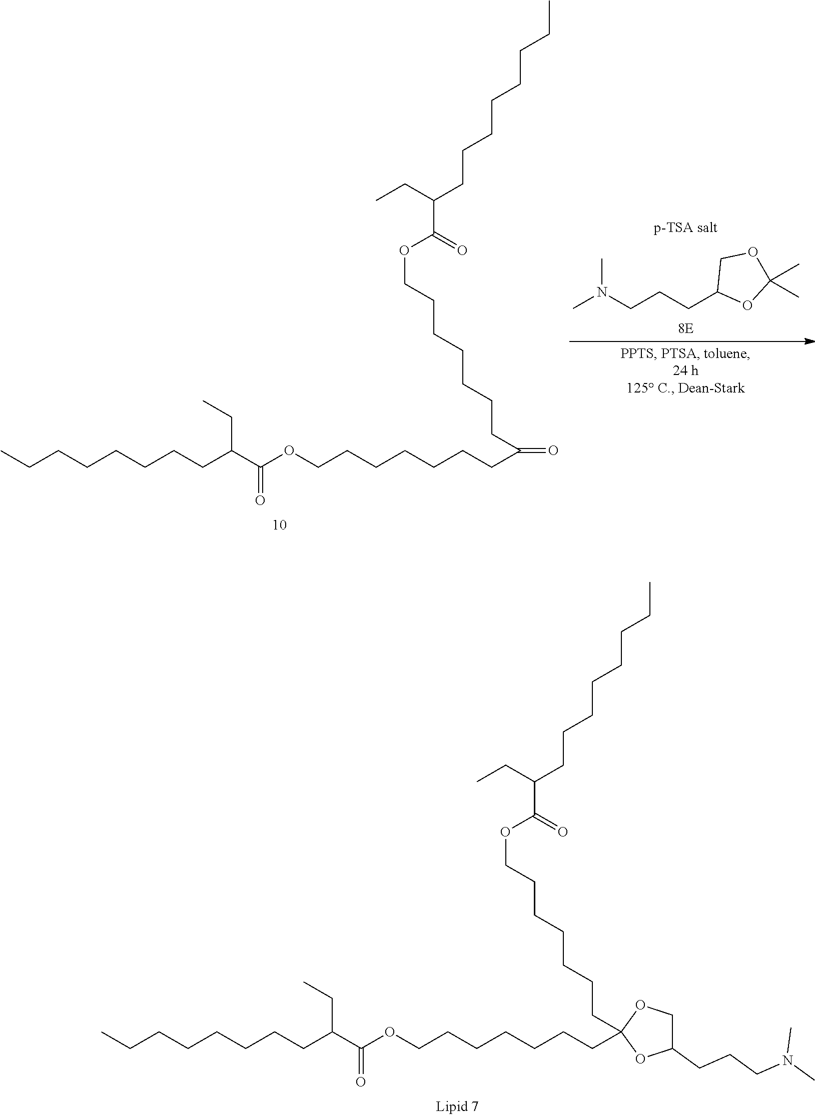

To a solution of compound 10 (900 mg, 1.44 mmol, 1 eq.) and compound 8E (992.58 mg, 2.89 mmol, 2 eq., TsOH salt) in toluene (10 mL) was added PPTS (181.52 mg, 722.31 μmol, 0.5 eq.) and PTSA (82.44 mg, 433.39 μmol, 0.3 eq.) at 25° C. The mixture was stirred at 125° C. for 12 hours equipped with Dean-Stark. The mixture was poured into sat. NaHCO₃ (aq., 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether (1% TEA)/Ethyl acetate (1% TEA)=5/1 to 3/1), followed by another column chromatography (SiO₂, A/B=100/1 to 5/1, phase A: PE:DCM=3:1, phase B: EtOAc, eluent with 0.1% TEA) to give Lipid 7 (420 mg, 38.26% yield, 99% purity) as a light yellow oil.

LCMS: m/z 752.8 [M+1]⁺. ¹HNMR: (400 MHz, CHLOROFORM-d) δ ppm 4.13-3.93 (m, 6H), 3.47-3.37 (m, 1H), 2.41-2.15 (m, 10H), 1.64-1.42 (m, 20H), 1.38-1.15 (m, 40H), 0.93-0.78 (m, 12H).

### Synthesis of Lipid 8

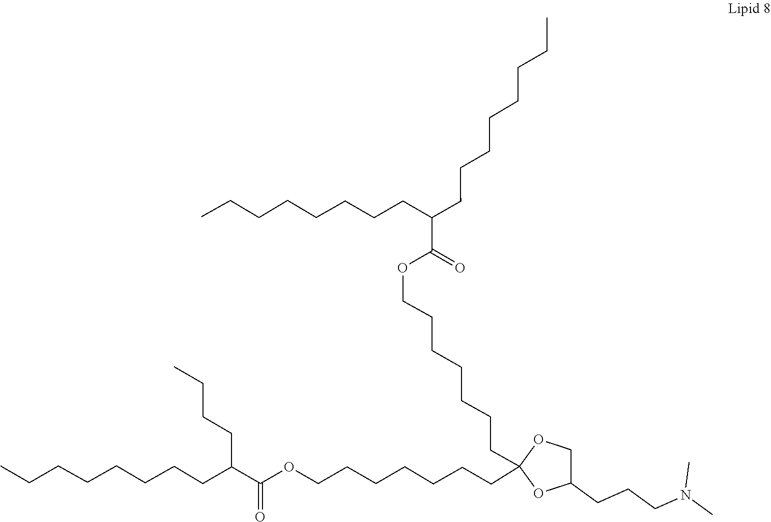 [SMILES: CCCCCCCCC(CCCC)C(=O)OCCCCCCCC1(CCCCCCCOC(=O)C(CCCCCCCC)CCCCCCCC)OCC(CCCN(C)C)O1]

A scheme for the synthesis of Lipid 8 is provided in Scheme 8 below.

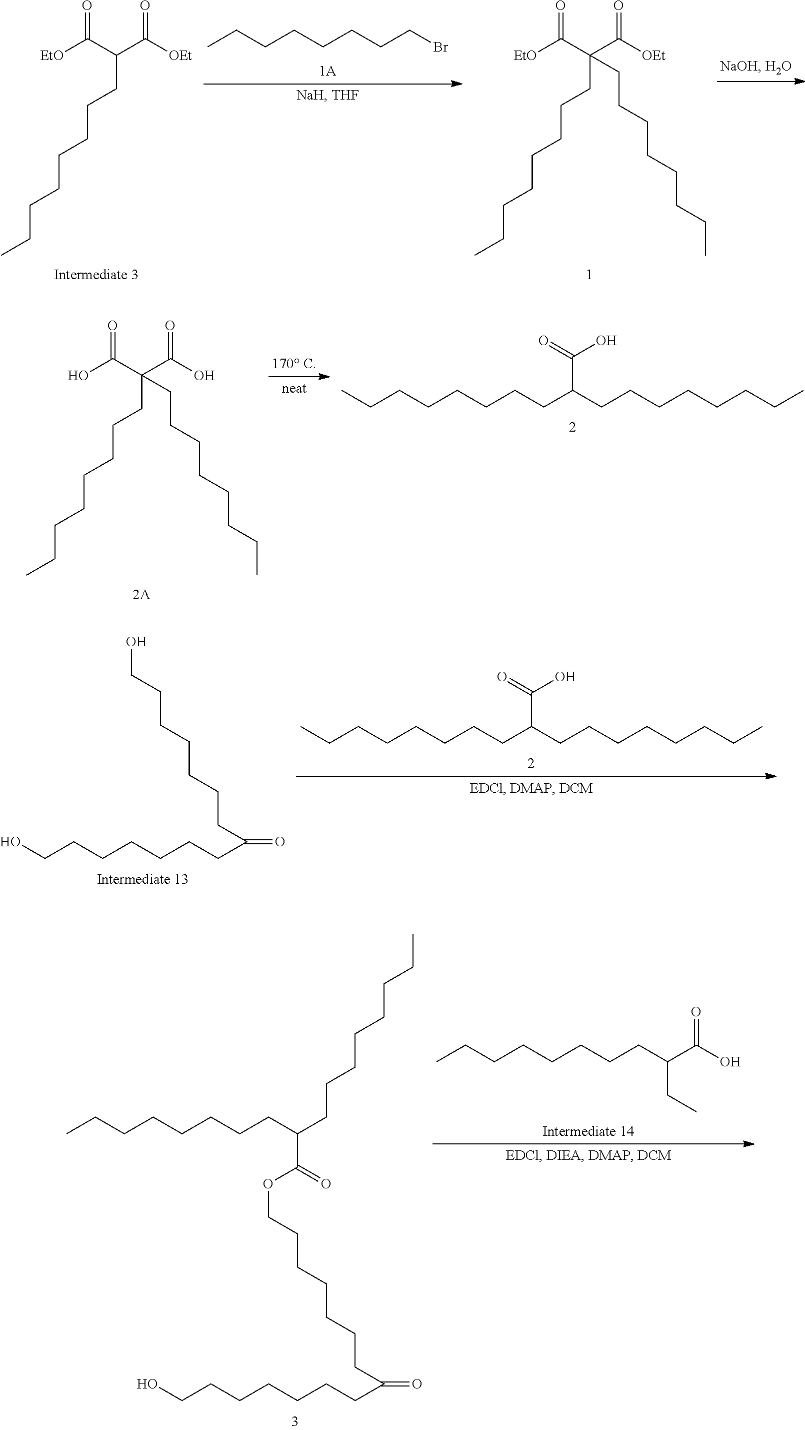

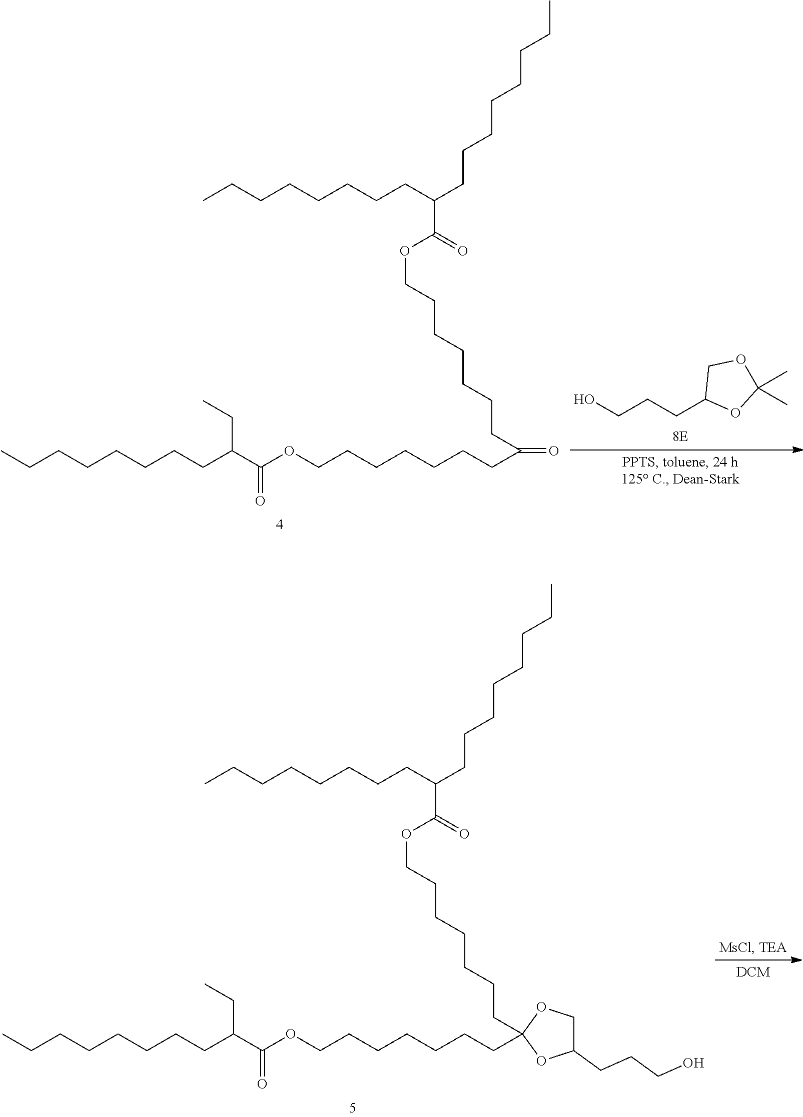 [SMILES: CCCCCCCCC(CC)C(=O)OCCCCCCCC(=O)CCCCCCCOC(=O)C(CCCCCCCC)CCCCCCCC ; CC1(C)OCC(CCCO)O1 ; CCCCCCCCC(CC)C(=O)OCCCCCCCC1(CCCCCCCOC(=O)C(CCCCCCCC)CCCCCCCC)OCC(CCCO)O1]

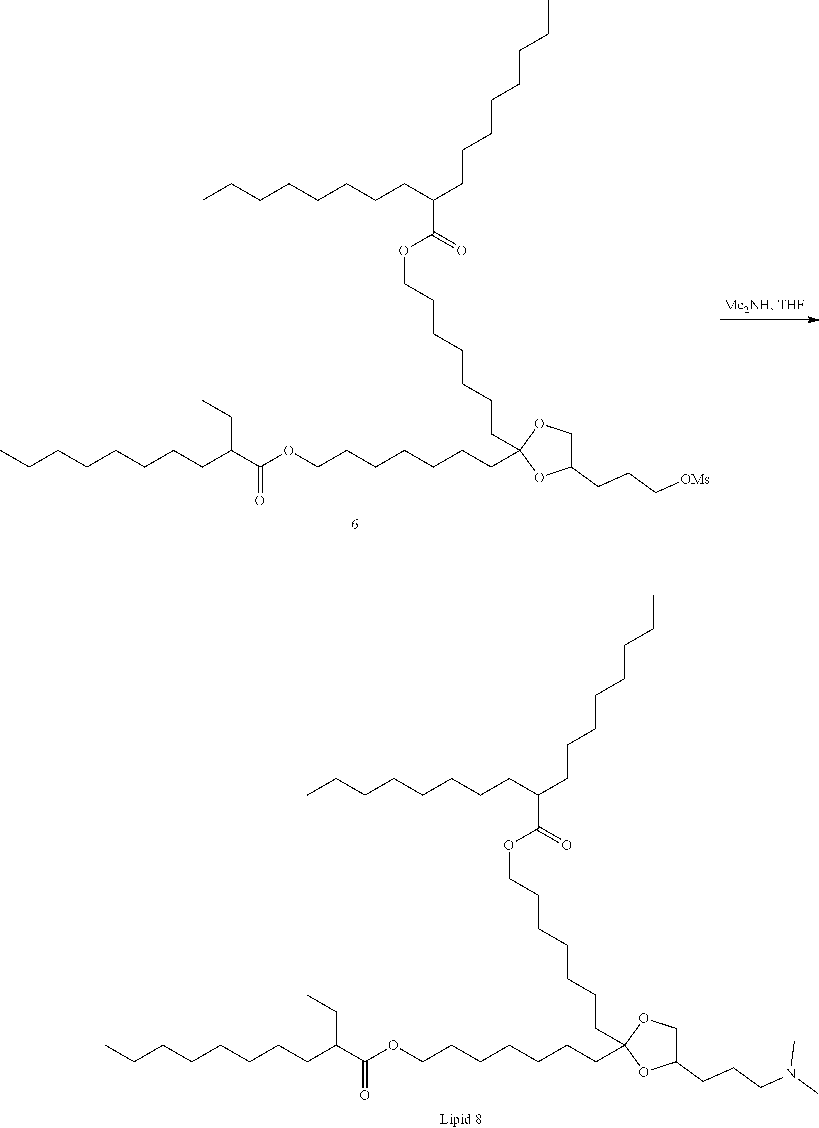 [SMILES: CCCCCCCCC(CC)C(=O)OCCCCCCCC1(CCCCCCCOC(=O)C(CCCCCCCC)CCCCCCCC)OCC(CCCOS(C)(=O)=O)O1 ; CCCCCCCCC(CC)C(=O)OCCCCCCCC1(CCCCCCCOC(=O)C(CCCCCCCC)CCCCCCCC)OCC(CCCN(C)C)O1]

### Procedure for Preparation of Compound 1

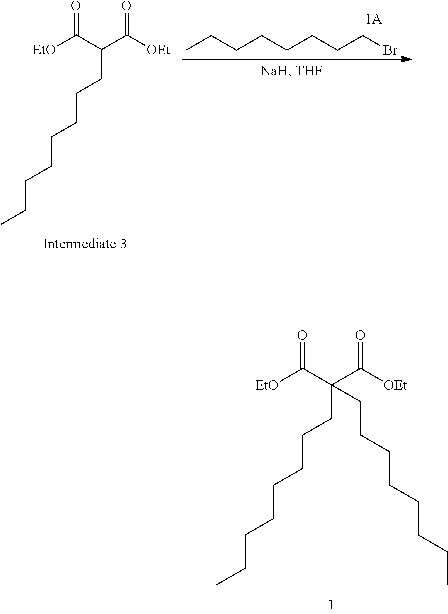

To a solution of Intermediate 3 (11 g, 40.38 mmol, 1 eq.) in THF (100 mL) added NaH (3.23 g, 80.77 mmol, 60% purity, 2 eq.) at 0° C. The mixture was stirred at 0° C. for 30 mins. Then compound 1A (15.60 g, 80.77 mmol, 14.05 mL, 2 eq.) was added at 25° C., and the mixture was stirred at 25° C. for addition 1.5 hour. The reaction mixture was quenched by addition of H₂O (100 mL) at 0° C., and then extracted with EtOAc (50 mL*3). The combined organic layers were washed with brine (60 mL), dried over anhydrous Na₂SO₄, filtered and concentrated under reduced pressure. The residue was purified by column chromatography (SiO₂, Petroleum ether/Ethyl acetate=20/1 to 10/1) to give compound 1 (8 g, 51.51% yield) as a colorless oil.

¹HNMR: (400 MHz, CHLOROFORM-d) δ ppm 4.17 (q, J=7.2 Hz, 4H), 1.96-1.77 (m, 4H), 1.34-1.07 (m, 31H), 0.88 (t, J=6.8 Hz, 6H).

### Procedure for Preparation of Compound 2A

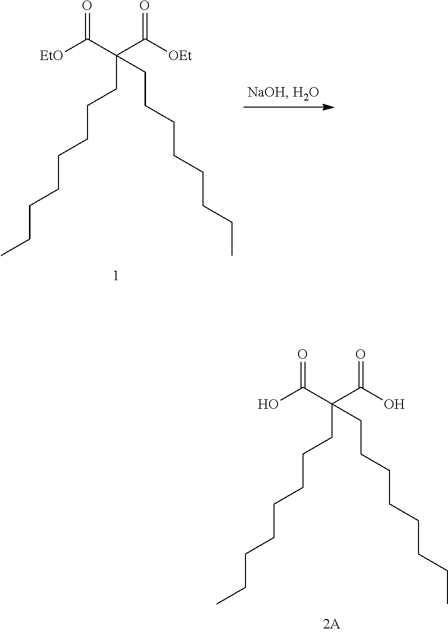 [SMILES: CCCCCCCCC(CCCCCCCC)(C(=O)OCC)C(=O)OCC ; CCCCCCCCC(CCCCCCCC)(C(=O)O)C(=O)O]

To a solution of compound 1 (8 g, 20.80 mmol, 1 eq.) in EtOH (40 mL) was added KOH aqueous solution (4 M, 36.40 mL, 7 eq.) dropwise at ambient temperature. The mixture was heated to 80° C. for 12 hours. The reaction mixture was diluted with H₂O (30 mL) at 0° C., and then extracted with EtO Ac (10 mL*3). The combined organic layers were washed with brine (20 mL), dried over anhydrous Na₂SO₄, filtered and concentrated under reduced pressure. The crude compound 2A (6.26 g) was obtained as a white solid and used directly for next step.

### Procedure for Preparation of Compound 2

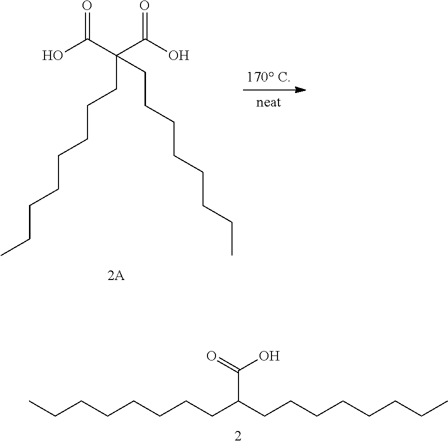 [SMILES: CCCCCCCCC(CCCCCCCC)(C(=O)O)C(=O)O ; CCCCCCCCC(CCCCCCCC)C(=O)O]

Compound 2A (6.26 g, 19.06 mmol, 1 eq.) was directly heated 170° C. for 3 hours without any solvent. The residue was directly purified by column chromatography (SiO₂, Petroleum ether/Ethyl acetate=10/1 to 1/1) to give compound 2 (4.2 g, 77.47% yield) as a colorless oil.

¹HNMR: (400 MHz, CHLOROFORM-d) δ ppm 2.35 (tt, J=5.2, 8.4 Hz, 1H), 1.69-1.57 (m, 2H), 1.54-1.42 (m, 2H), 1.39-1.20 (m, 24H), 0.93-0.84 (m, 6H).

### Procedure for Preparation of Compound 3

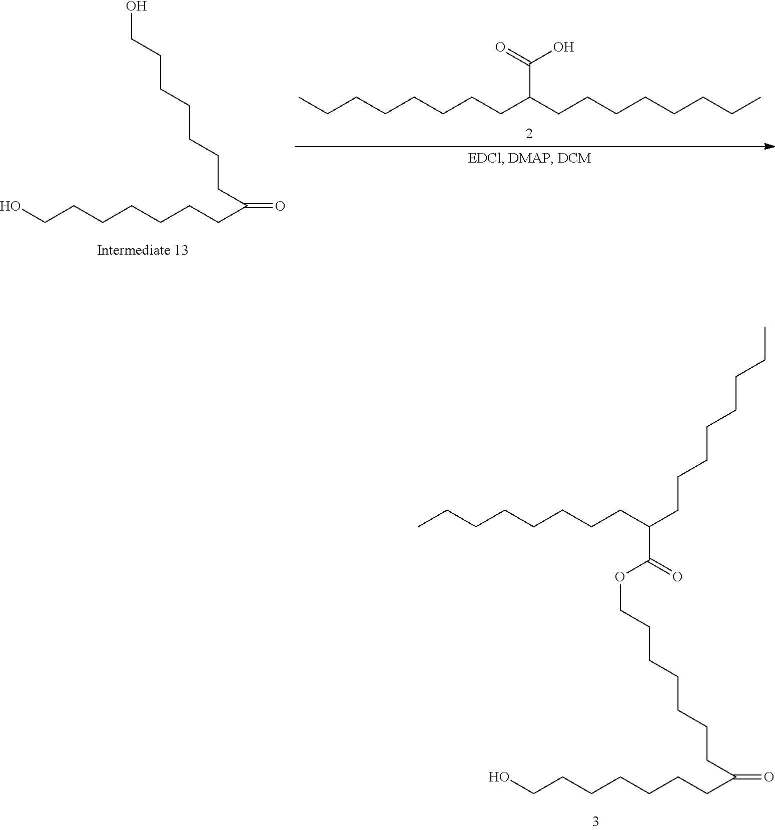

To a solution of Intermediate 13 (1.00 g, 3.88 mmol, 1.3 eq.) in DMF (15 mL) was added EDCI (750.36 mg, 3.91 mmol, 1.31 eq.), compound 2 (850 mg, 2.99 mmol, 1 eq.) and DMAP (375.98 mg, 3.08 mmol, 1.03 eq.). The mixture was stirred at 25° C. for 12 hours. The reaction mixture was poured into ice-water (w/w=1/1, 30 mL) and stirred for 2 mins, and then extracted with DCM (30 mL*3). The combined organic phases were washed with brine (50 mL), dried over anhydrous Na₂SO₄, filtered and concentrated in vacuo. The residue was purified by column chromatography (SiO₂, Petroleum ether/Ethyl acetate=5/1 to 3/1) to give compound 3 (610 mg, 38.90% yield) as colorless oil. (One spot on TLC but not very clean on HNMR)

¹HNMR: (400 MHz, CHLOROFORM-d) δ ppm 4.10-4.02 (m, 3H), 3.68-3.60 (m, 2H), 2.39 (t, J=7.2 Hz, 4H), 2.34-2.26 (m, 1H), 1.67-1.50 (m, 12H), 1.42-1.18 (m, 36H), 0.88 (t, J=6.8 Hz, 6H).

### Procedure for Preparation of Compound 4

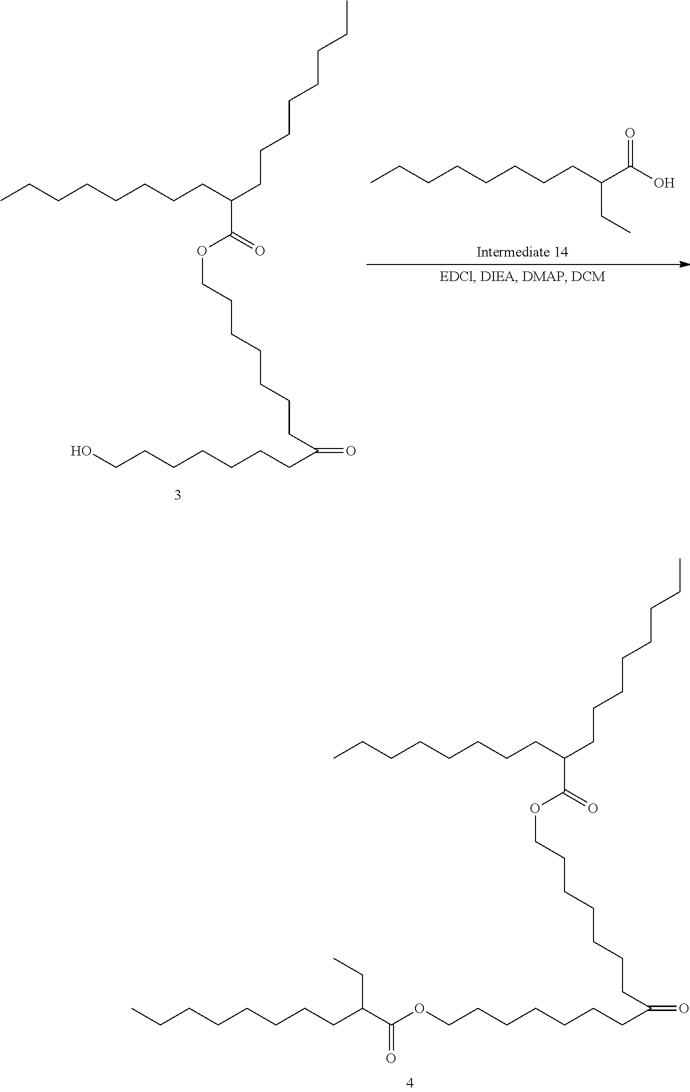 [SMILES: CCCCCCCCC(CCCCCCCC)C(=O)OCCCCCCCC(=O)CCCCCCCO ; CCCCCCCCC(CC)C(=O)O ; CCCCCCCCC(CC)C(=O)OCCCCCCCC(=O)CCCCCCCOC(=O)C(CCCCCCCC)CCCCCCCC]

To a mixture of compound 3 (510 mg, 971.69 μmol, 1 eq.) and intermediate 14 (214.11 mg, 1.07 mmol, 1.1 eq.) in DCM (15 mL) was added DMAP (118.71 mg, 971.69 μmol, 1 eg.), DIEA (502.34 mg, 3.89 mmol, 677.00 μL, 4 eq.) and EDCI (372.55 mg, 1.94 mmol, 2 eq.). The mixture was stirred at 25° C. for 12 hours. The reaction mixture was poured into ice-water (w/w=1/1, 30 mL) and stirred for 2 mins, and then extracted with DCM (30 mL*3). The combined organic phases were washed with brine (50 mL), dried over anhydrous Na₂SO₄, filtered and concentrated in vacuo. The residue was purified by column chromatography (SiO₂, Petroleum ether/Ethyl acetate=10/1 to 5/1) to give compound 4 (500 mg, 72.77% yield) as a colorless oil.

¹HNMR: (400 MHz, CHLOROFORM-d) δ ppm 4.06 (dt, J=2.8, 6.8 Hz, 4H), 2.39 (t, J=7.2 Hz, 4H), 2.33-2.21 (m, 2H), 1.69-1.49 (m, 13H), 1.46-1.19 (m, 49H), 0.94-0.79 (m, 12H).

### Procedure for Preparation of Compound 5

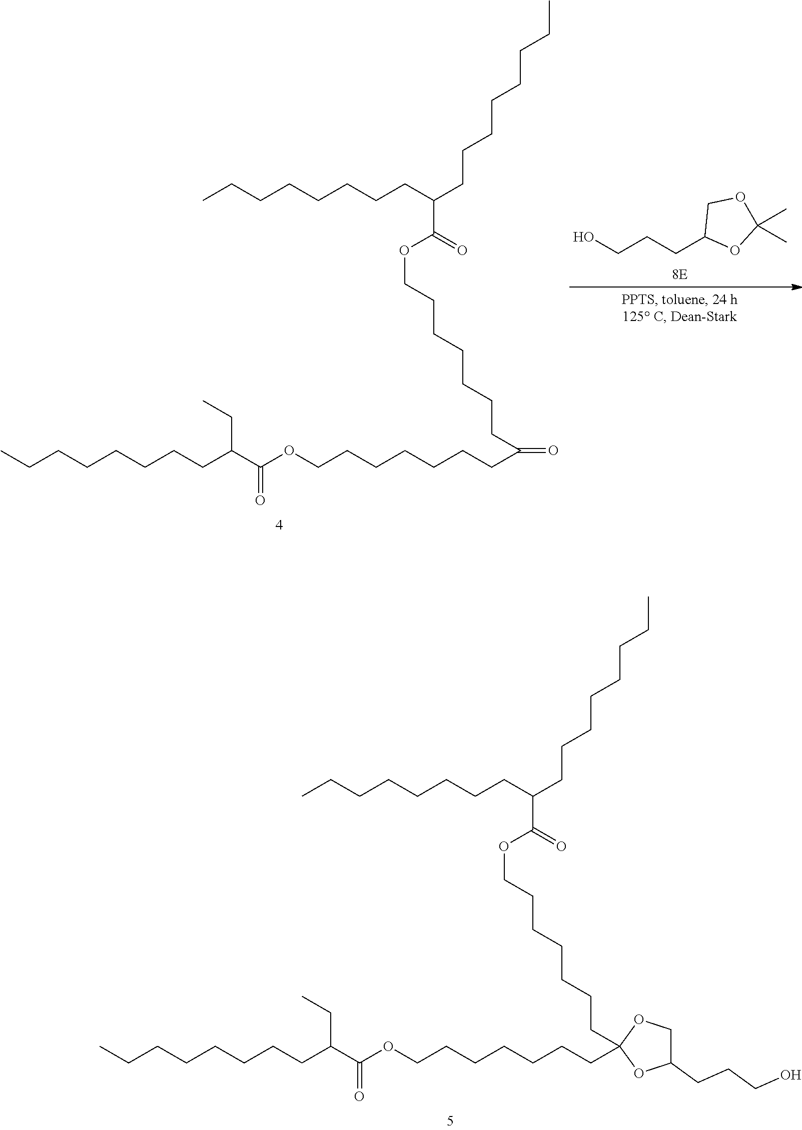 [SMILES: CCCCCCCCC(CC)C(=O)OCCCCCCCC(=O)CCCCCCCOC(=O)C(CCCCCCCC)CCCCCCCC ; CC1(C)OCC(CCCO)O1 ; CCCCCCCCC(CC)C(=O)OCCCCCCCC1(CCCCCCCOC(=O)C(CCCCCCCC)CCCCCCCC)OCC(CCCO)O1]

To a mixture of compound 4 (450 mg, 636.35 μmol, 1 eq.) and compound 8E (254.87 mg, 1.59 mmol, 2.5 eq.) in toluene (20 mL) was added PPTS (47.97 mg, 190.91 μmol, 0.3 eq.) at 25° C. The mixture was stirred at 125° C. for 24 hours equipped with Dean-stark. The reaction mixture was poured into ice-water (w/w=1/1, 50 mL) and stirred for 2 mins, and then extracted with DCM (10 mL*3). The combined organic phases were washed with brine (10 mL*3), dried over anhydrous Na₂SO₄, filtered and concentrated in vacuo. The residue was purified by column chromatography (SiO₂, Petroleum ether/Ethyl acetate=5/1 to 3/1) to give compound 5 (280 mg, 54.37% yield) as a light yellow oil.

¹HNMR: (400 MHz, CHLOROFORM-d) δ ppm 4.20-4.01 (m, 6H), 3.80-3.65 (m, 2H), 3.57-3.44 (m, 1H), 2.40-2.19 (m, 2H), 1.79-1.53 (m, 18H), 1.48-1.20 (m, 54H), 1.01-0.79 (m, 12H)

### Procedure for Preparation of Compound 6

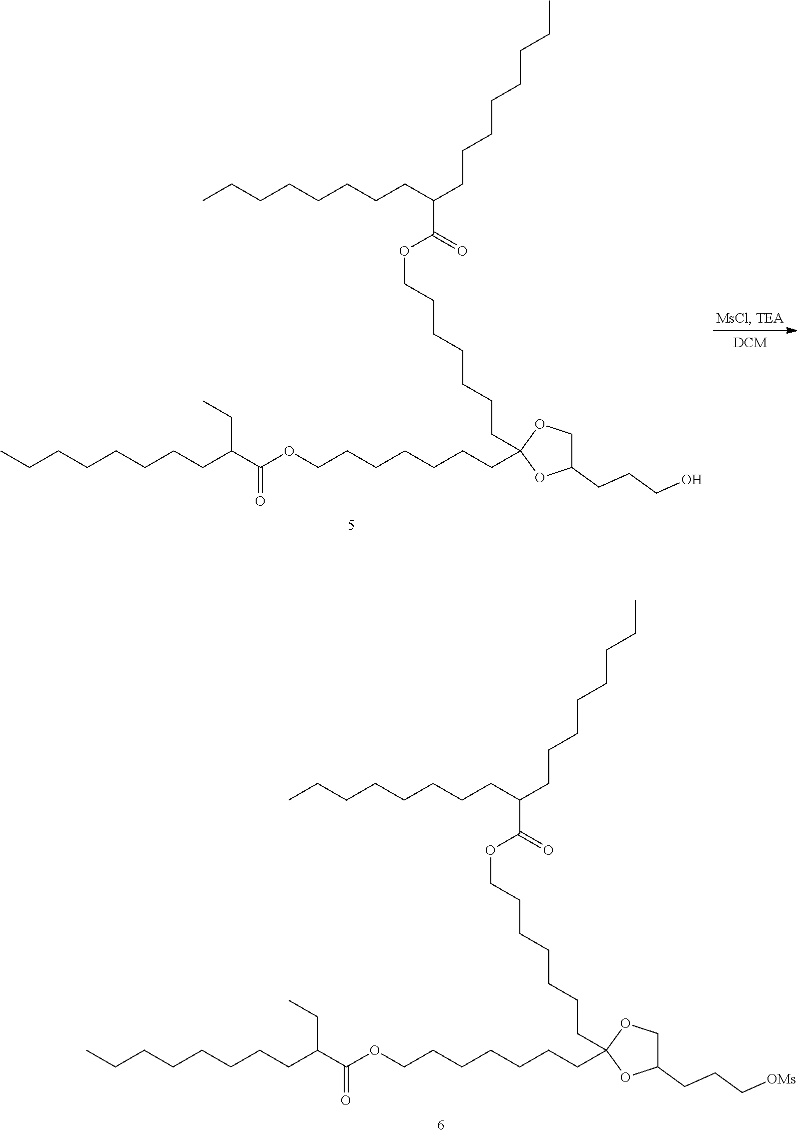 [SMILES: CCCCCCCCC(CC)C(=O)OCCCCCCCC1(CCCCCCCOC(=O)C(CCCCCCCC)CCCCCCCC)OCC(CCCO)O1 ; CCCCCCCCC(CC)C(=O)OCCCCCCCC1(CCCCCCCOC(=O)C(CCCCCCCC)CCCCCCCC)OCC(CCCOS(C)(=O)=O)O1]

To a solution of compound 5 (280 mg, 345.98 μmol, 1 eq.) in DCM (5 mL) was added TEA (175.05 mg, 1.73 mmol, 240.78 μL, 5 eq.) and MsCl (118.90 mg, 1.04 mmol, 80.34 μL, 3 eq.) at 25° C. The mixture was stirred at 25° C. for 12 hours. The reaction mixture was poured into ice-water (w/w=1/1, 30 mL) and stirred for 2 mins, and then extracted with DCM (30 mL*3). The combined organic phases were washed with brine (30 mL), dried over anhydrous Na₂SO₄, filtered and concentrated in vacuo. The residue was purified by column chromatography (SiO₂, Petroleum ether/Ethyl acetate=5/1 to 3/1) to give compound 6 (290 mg, 94.46% yield) as a light yellow oil.

¹HNMR: (400 MHz, CHLOROFORM-d) δ ppm 4.21 (q, J=6.6 Hz, 2H), 3.99 (dt, J=3.2, 6.8 Hz, 6H), 3.49-3.34 (m, 1H), 2.94 (s, 3H), 2.31-2.11 (m, 2H), 1.93-1.68 (m, 2H), 1.66-1.05 (m, 70H), 0.92-0.71 (m, 12H).

### Procedure for Preparation of Lipid 8

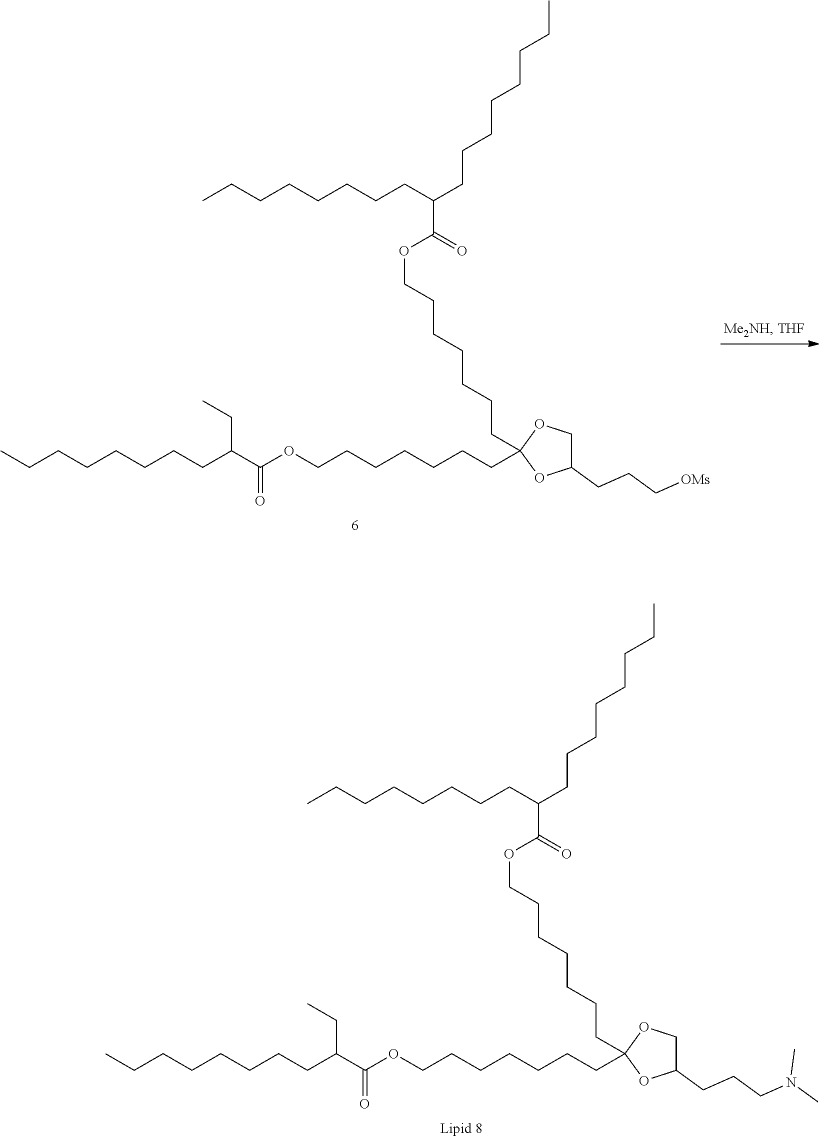 [SMILES: CCCCCCCCC(CC)C(=O)OCCCCCCCC1(CCCCCCCOC(=O)C(CCCCCCCC)CCCCCCCC)OCC(CCCOS(C)(=O)=O)O1 ; CCCCCCCCC(CC)C(=O)OCCCCCCCC1(CCCCCCCOC(=O)C(CCCCCCCC)CCCCCCCC)OCC(CCCN(C)C)O1]

To a solution of compound 6 (260 mg, 293.00 μmol, 1 eq.) in THF (1 mL) was added Me₂NH (2 M in THF, 13 mL, 88.74 eq.) at 25° C. The mixture was stirred at 40° C. for 12 hours. The reaction mixture was poured into ice-water (w/w=1/1, 30 mL) and then extracted with DCM (30 mL*3). The combined organic phases were washed with brine (30 mL), dried over anhydrous Na₂SO₄, filtered and concentrated in vacuo. The residue was purified by column chromatography (SiO₂, Petroleum ether/Ethyl acetate=10/1 to 3/1, 0.1% of TEA in the eluent) twice to give Lipid 8 (220 mg, 86.11% yield, 95.92% purity) as a light-yellow oil.

Special LCMS: m/z 836.9 [M+1]⁺. ¹HNMR: (400 MHz, CHLOROFORM-d) δ ppm 4.28-3.93 (m, 6H), 3.47 (t, J=6.8 Hz, 1H), 2.45-2.17 (m, 10H), 1.72-1.45 (m, 20H), 1.41-1.20 (m, 52H), 0.97-0.83 (m, 12H).

### Synthesis of Lipid 9

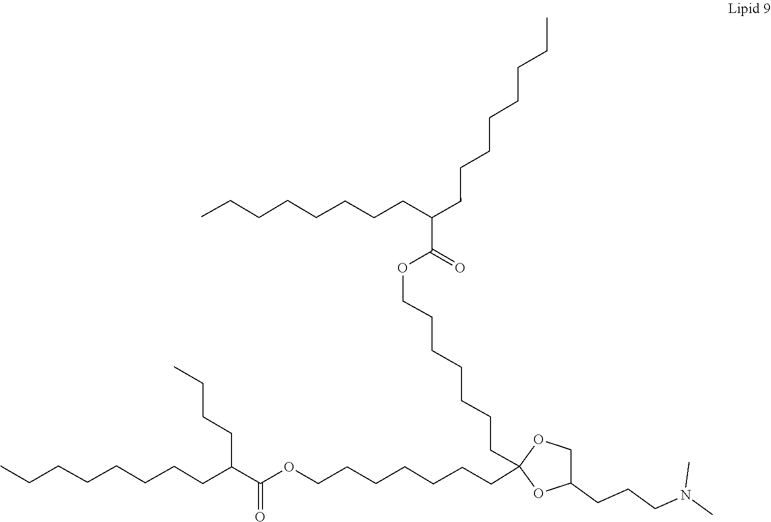 [SMILES: CCCCCCCCC(CCCC)C(=O)OCCCCCCCC1(CCCCCCCOC(=O)C(CCCCCCCC)CCCCCCCC)OCC(CCCN(C)C)O1]

A scheme for the synthesis of Lipid 9 is provided in Scheme 9 below.

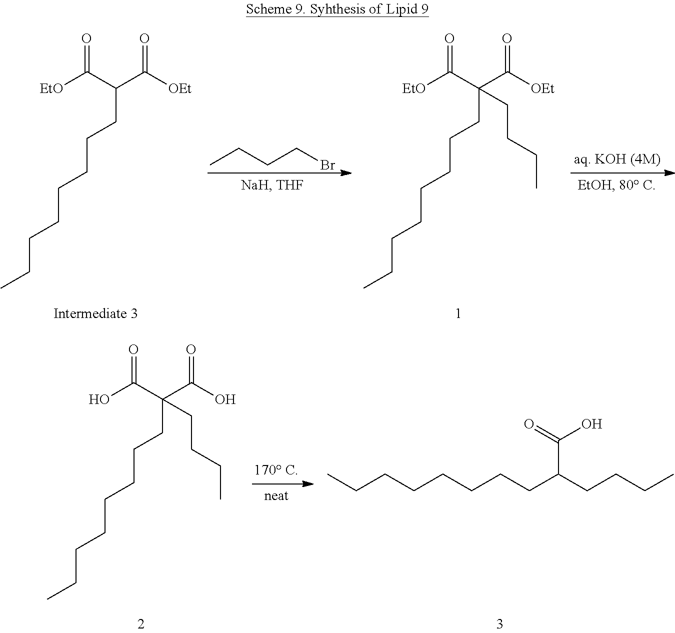

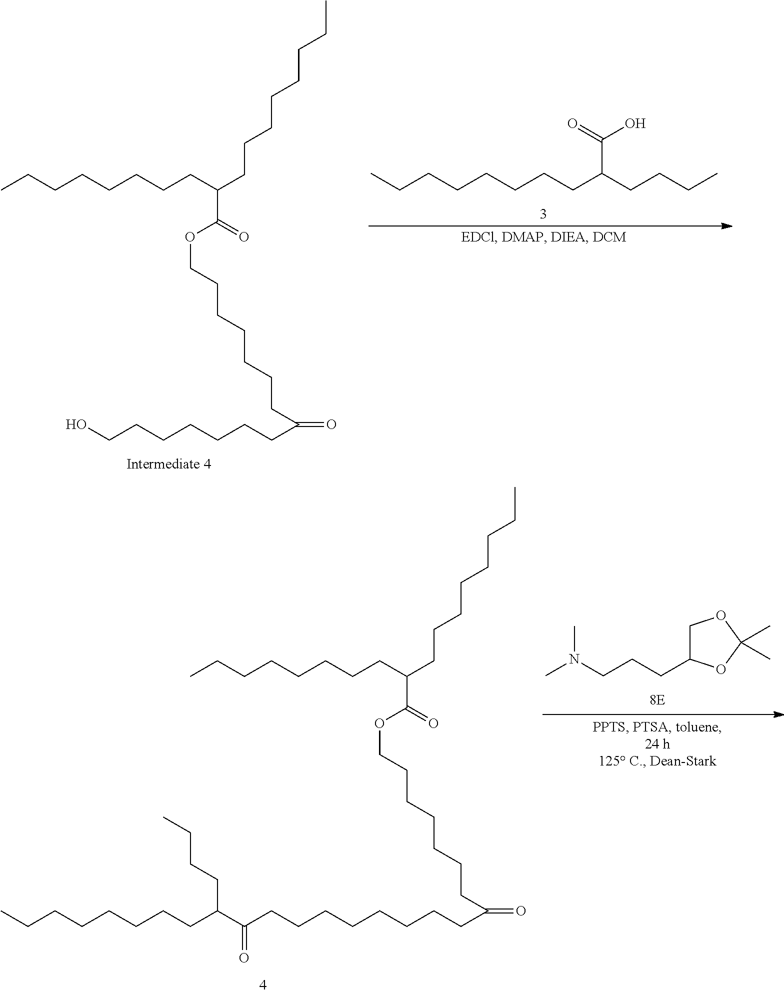

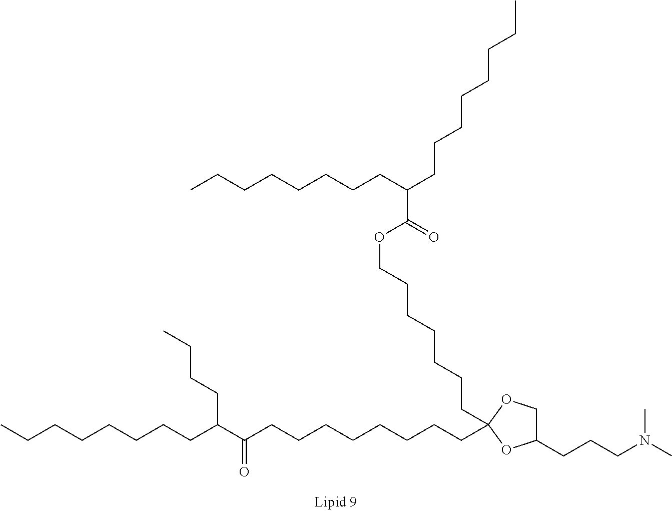 [SMILES: CCCCCCCCC(CCCC)C(=O)CCCCCCCCC1(CCCCCCCOC(=O)C(CCCCCCCC)CCCCCCCC)OCC(CCCN(C)C)O1]

### Procedure for Preparation of Compound 1

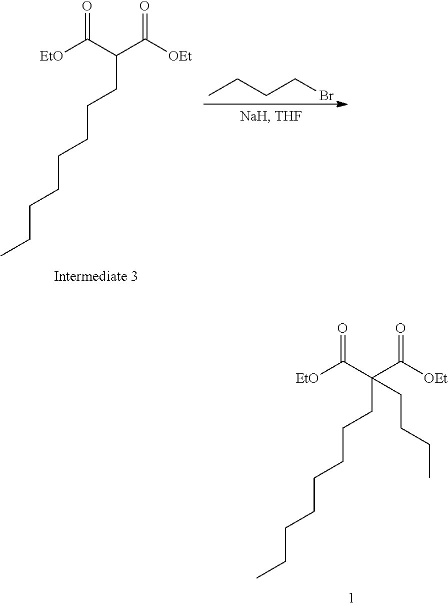

To a solution of intermediate 3 (5 g, 31.22 mmol, 4.74 mL, 1 eq.) in EtOH (50 mL) was added NaH (660.78 mg, 16.52 mmol, 60% purity, 1.5 eq.) in portions at 0° C. Then 1-bromobutane (6.03 g, 31.22 mmol, 5.43 mL, 1 eq.) was added dropwise at 25° C. The mixture was stirred at 60° C. for 2 hours. The mixture was poured into ice-water (w/w=1/1, 50 mL) and stirred for 2 mins. The aqueous phase was extracted with ethyl acetate (50 mL*3). The combined organic phases were washed with brine (60 mL), dried over anhydrous Na₂SO₄, filtered and concentrated under vacuum. The residue was purified by column chromatography (SiO₂, Petroleum ether/Ethyl acetate=20/1 to 10/1) to give compound 1 (5.2 g, 74.29% yield) as a yellow oil.

¹HNMR: (400 MHz, CHLOROFORM-d) δ ppm 4.19 (q, J=7.2 Hz, 4H), 1.94-1.83 (m, 4H), 1.38-1.23 (m, 18H), 1.21-1.09 (m, 4H), 0.91 (q, J=7.2 Hz, 6H).

### Procedure for Preparation of Compound 2

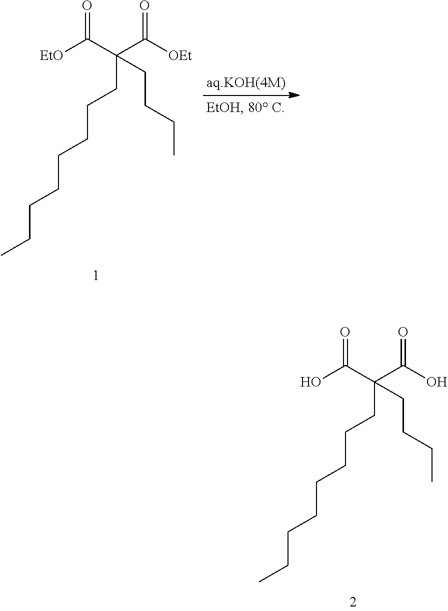 [SMILES: CCCCCCCCC(CCCC)(C(=O)OCC)C(=O)OCC ; CCCCCCCCC(CCCC)(C(=O)O)C(=O)O]

To a solution of compound 1 (5.2 g, 15.83 mmol, 1 eq.) in EtOH (25 mL) was added KOH (4 M aqueous solution, 27.70 mL, 7 eq.) at 25° C. The mixture was stirred at 80° C. for 12 hours. The reaction mixture was quenched by addition of HCl (2M aqueous solution, 10 mL) at 0° C., and then extracted with EtOAc (40 mL*3). The combined organic layers were washed with brine (60 mL), dried over anhydrous Na₂SO₄, filtered and concentrated under reduced pressure to give compound 2 (4.3 g, crude) as a yellow solid, which was used directly for next step without further purification.

### Procedure for Preparation of Compound 3

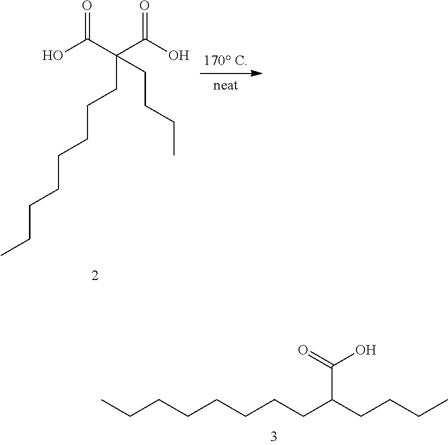 [SMILES: CCCCCCCCC(CCCC)(C(=O)O)C(=O)O ; CCCCCCCCC(CCCC)C(=O)O]

The crude compound 2 (4.3 g, 15.79 mmol, 1 eq.) in a 50 mL flask without solvent was heated to 170° C. for 4 hours. The residue was directly purified by column chromatography (SiO₂, Petroleum ether/Ethyl acetate=20/1 to 10/1) to give compound 3 (3.2 g, 88.76% yield) as a yellow oil.

¹HNMR: (400 MHz, CHLOROFORM-d) δ ppm 2.27 (ddd, J=3.2, 5.4, 8.8 Hz, 1H), 1.63-1.50 (m, 2H), 1.47-1.34 (m, 2H), 1.32-1.09 (m, 16H), 0.82 (q, J=7.2 Hz, 6H).

### Procedure for Preparation of Compound 4

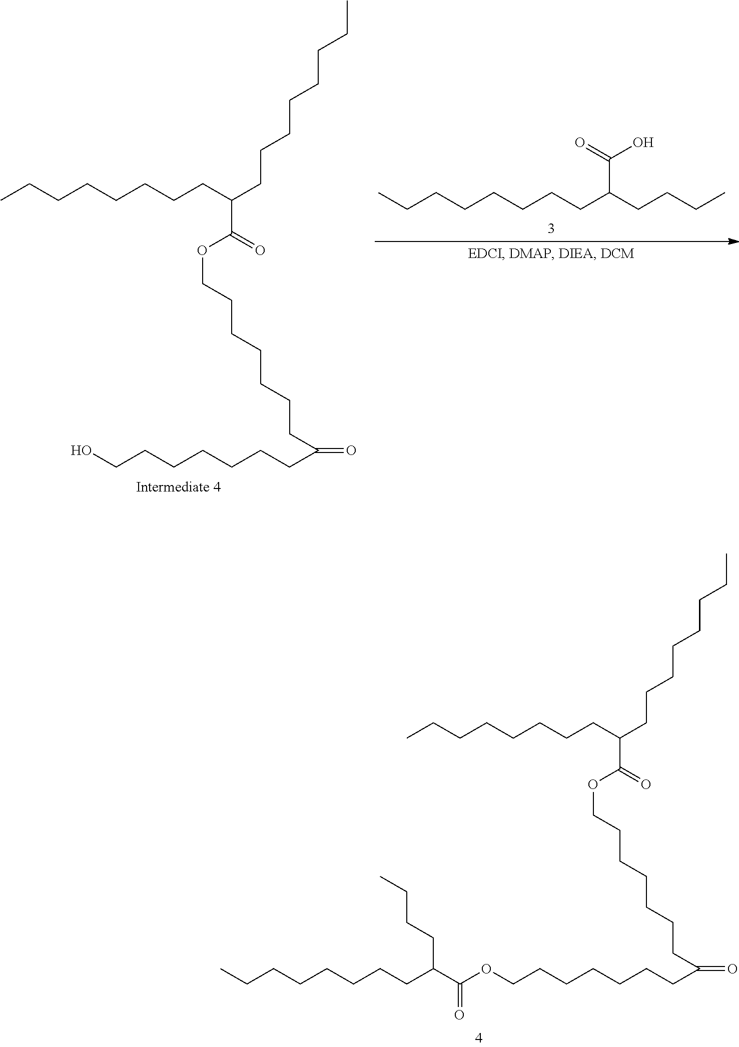 [SMILES: CCCCCCCCC(CCCCCCCC)C(=O)OCCCCCCCC(=O)CCCCCCCO ; CCCCCCCCC(CCCC)C(=O)O ; CCCCCCCCC(CCCC)C(=O)OCCCCCCCC(=O)CCCCCCCOC(=O)C(CCCCCCCC)CCCCCCCC]

To a solution of intermediate 4 (710 mg, 1.35 mmol, 1 eq.) and compound 3 (339.82 mg, 1.49 mmol, 1.1 eq.) in DCM (10 mL) was added DIEA (699.33 mg, 5.41 mmol, 942.49 μL, 4 eq.), DMAP (165.26 mg, 1.35 mmol, 1 eq.) and EDCI (518.65 mg, 2.71 mmol, 2 eq.) at 25° C. The mixture was stirred at 40° C. for 12 hours. The mixture was poured into ice-water (w/w=1/1, 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether/Ethyl acetate=20/1 to 10/1) to give compound 4 (780 mg, 78.43% yield) as a light yellow oil.

¹HNMR: (400 MHz, CHLOROFORM-d) δ ppm 4.08 (t, J=6.4 Hz, 4H), 2.40 (t, J=7.2 Hz, 4H), 2.35-2.27 (m, 2H), 1.66-1.55 (m, 16H), 1.46 (br d, J=5.4 Hz, 4H), 1.37-1.24 (m, 48H), 0.90 (t, J=6.4 Hz, 12H).

### Procedure for Preparation of Lipid 9

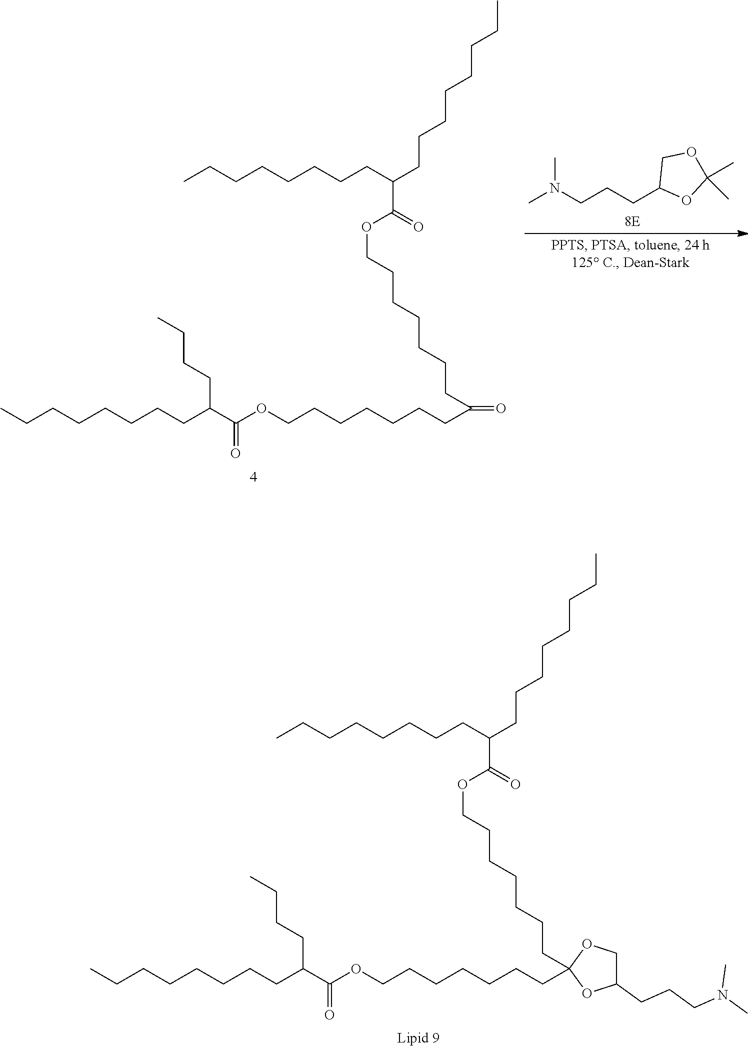

To a solution of compound 4 (764 mg, 1.04 mmol, 1 eq.) and compound 8E (497.48 mg, 2.08 mmol, 2 eq., TsOH salt) in toluene (20 mL) was added PPTS (130.57 mg, 519.58 μmol, 0.5 eq.) and PTSA (59.30 mg, 311.75 μmol, 0.3 eq.) at 25° C. The mixture was stirred at 125° C. for 12 hours equipped with Dean-Stark. The mixture was poured into sat. NaHCO₃ (aq., 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether (1% TEA)/Ethyl acetate (1% TEA)=5/1 to 3/1), followed by another column purification (SiO₂, A/B=100/1 to 5/1, phase A: PE:DCM=3:1, phase B: EtOAc, eluent with 0.1% TEA) to give Lipid 9 (350 mg, 38.11% yield, 97.8% purity) as a yellow oil.

LCMS: m/z 865.0 [M+1]⁺. ¹HNMR: (400 MHz, CHLOROFORM-d) δ ppm 4.13-3.98 (m, 6H), 3.46 (s, 1H), 2.39-2.17 (m, 10H), 1.67-1.54 (m, 16H), 1.45 (br d, J=5.4 Hz, 4H), 1.39-1.20 (m, 56H), 0.89 (t, J=6.4 Hz, 12H).

### Synthesis of Lipid 10

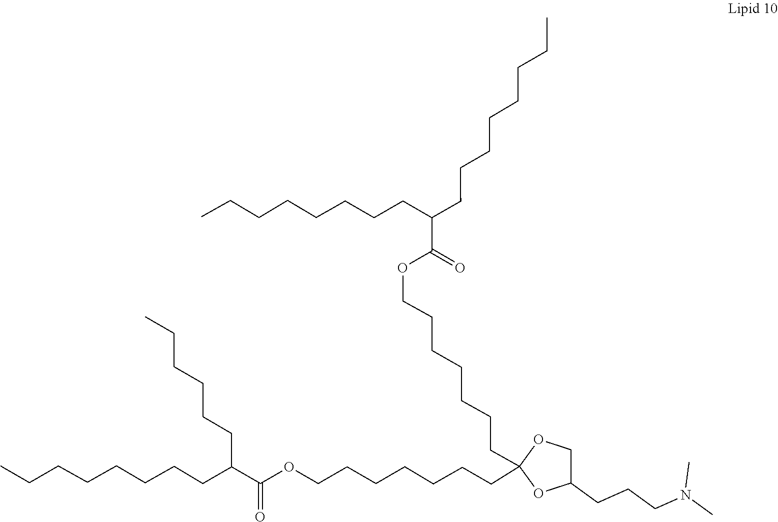 [SMILES: CCCCCCCCC(CCCCCC)C(=O)OCCCCCCCC1(CCCCCCCOC(=O)C(CCCCCCCC)CCCCCCCC)OCC(CCCN(C)C)O1]

A scheme for the synthesis of Lipid 10 is provided in Scheme 10 below.

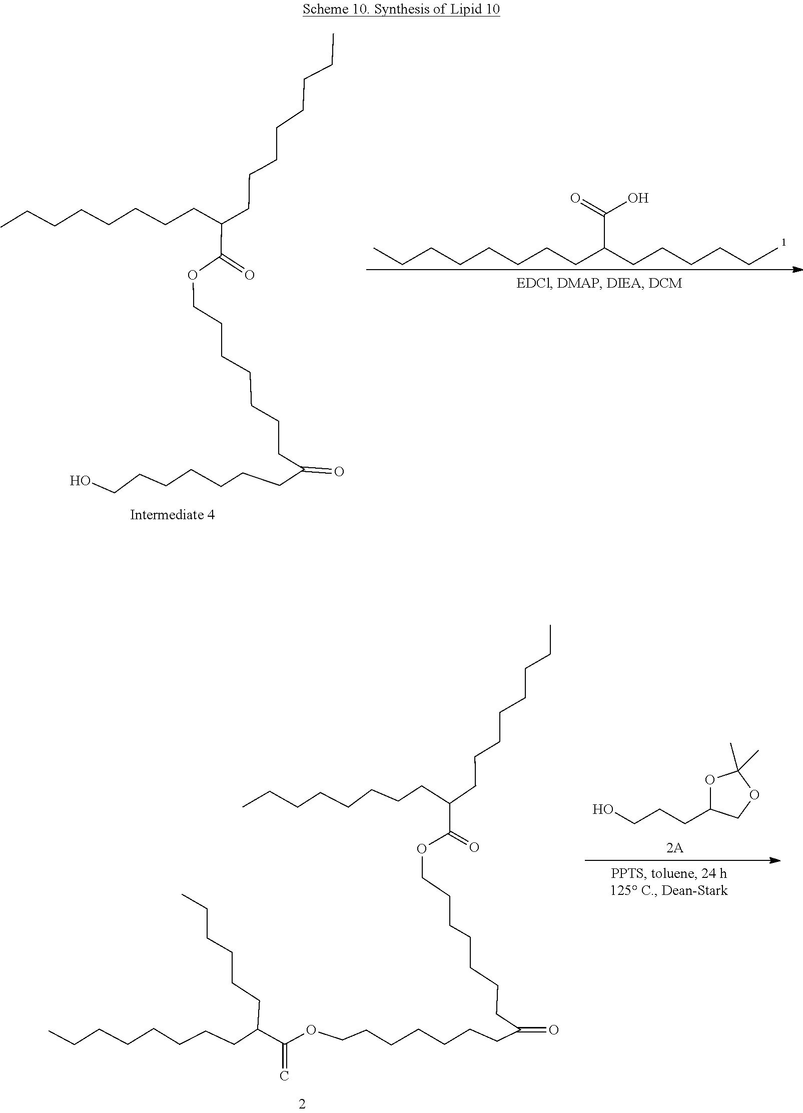

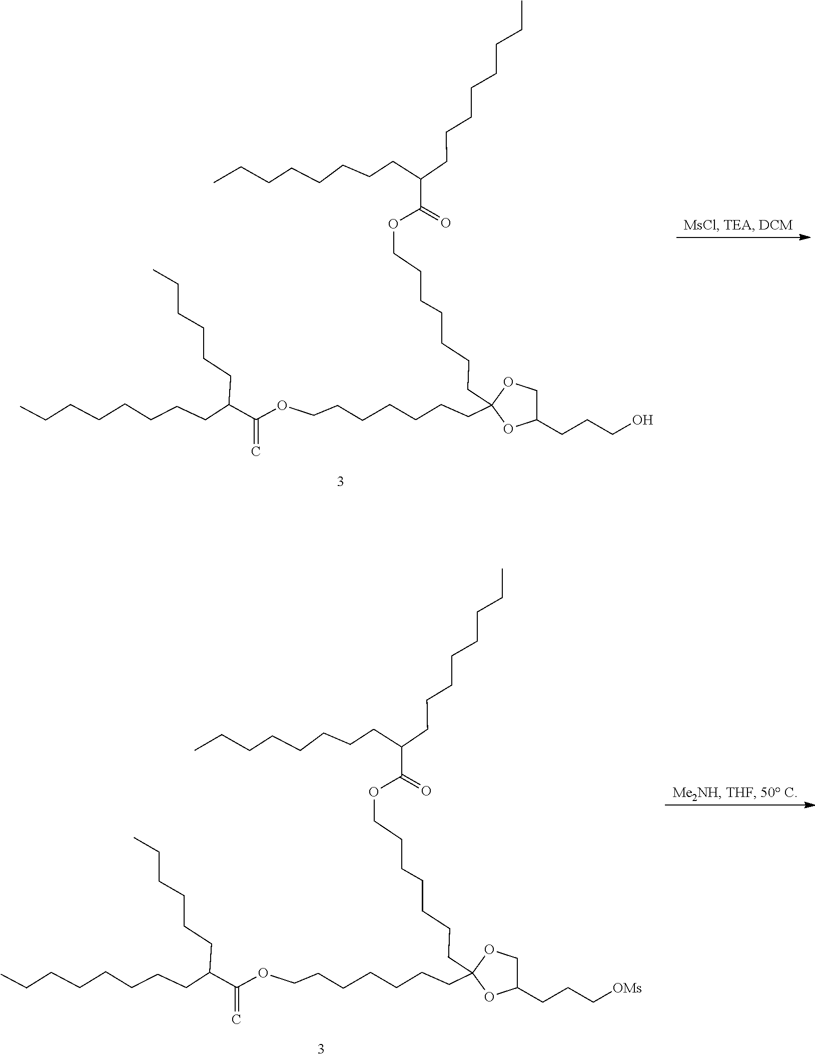

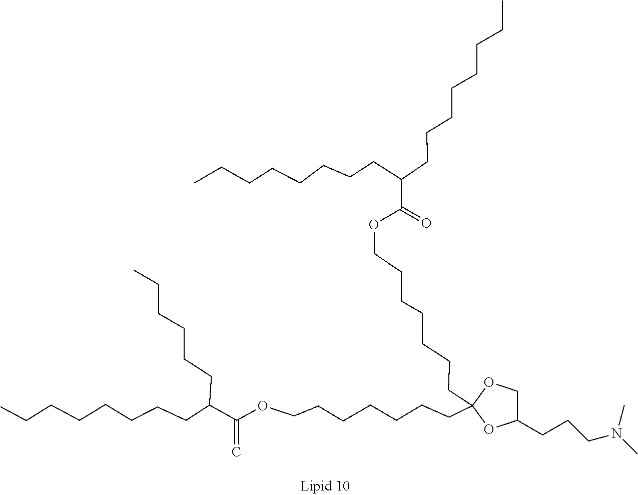

### Procedure for Preparation of Compound 2

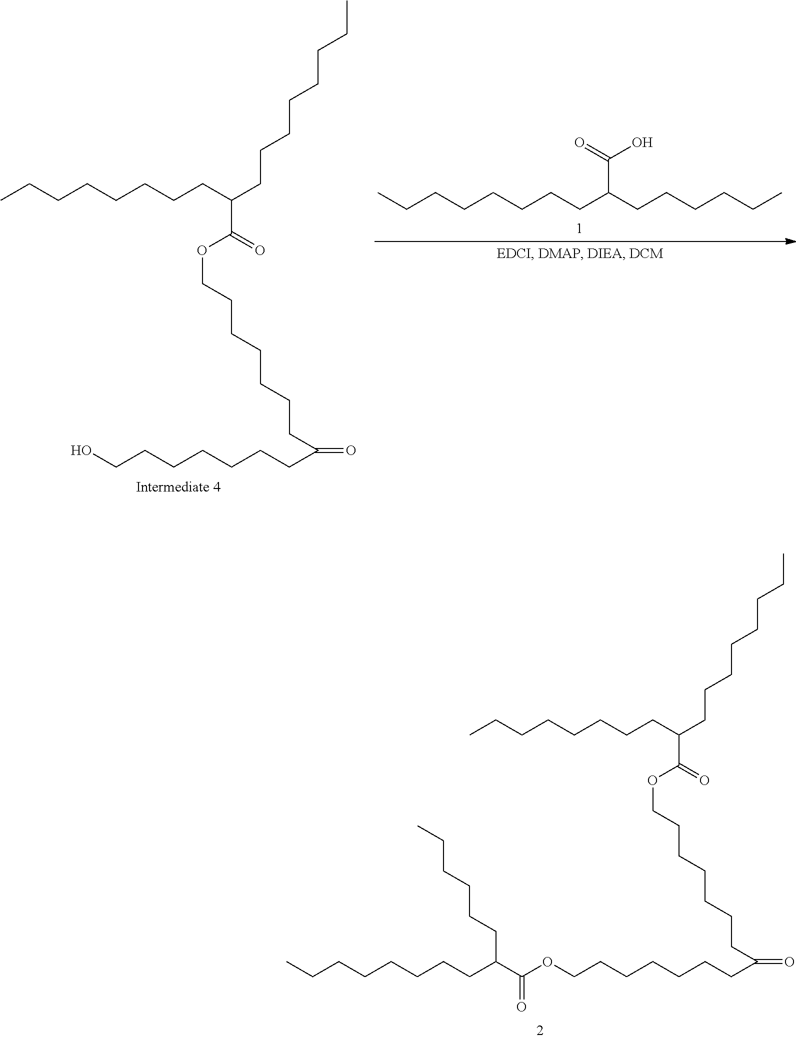 [SMILES: CCCCCCCCC(CCCCCCCC)C(=O)OCCCCCCCC(=O)CCCCCCCO ; CCCCCCCCC(CCCCCC)C(=O)O ; CCCCCCCCC(CCCCCC)C(=O)OCCCCCCCC(=O)CCCCCCCOC(=O)C(CCCCCCCC)CCCCCCCC]

To a solution of intermediate 4 (866 mg, 1.65 mmol, 1 eq.) and compound 1 (465.40 mg, 1.81 mmol, 1.1 eq.) in DCM (15 mL) was added EDCI (632.60 mg, 3.30 mmol, 2 eq.), DIEA (852.99 mg, 6.60 mmol, 1.15 mL, 4 eq.) and DMAP (201.57 mg, 1.65 mmol, 1 eq.) at 25° C. The mixture was stirred at 40° C. for 12 hours. The mixture was poured into ice-water (w/w=1/1, 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether/Ethyl acetate=20/1 to 10/1) to give compound 2 (541 mg, 42.96% yield) as a colorless oil.

¹HNMR: (400 MHz, CHLOROFORM-d) δ ppm 4.09-4.03 (m, 4H), 2.41-2.36 (s, 4H), 2.35-2.27 (m, 2H), 1.60-1.48 (s, 15H), 1.47-1.38 (m, 5H), 1.37-1.21 (m, 56H), 0.89 (t, J=6.8 Hz, 12H).

### Procedure for Preparation of Compound 3

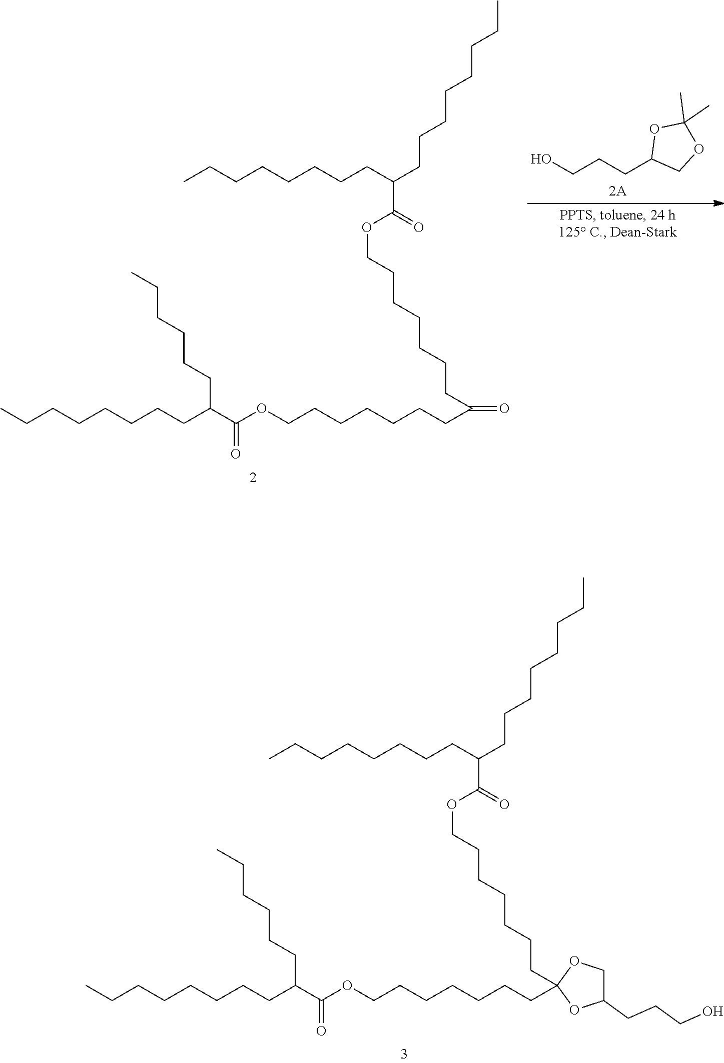 [SMILES: CCCCCCCCC(CCCCCC)C(=O)OCCCCCCCC(=O)CCCCCCCOC(=O)C(CCCCCCCC)CCCCCCCC ; CC1(C)OCC(CCCO)O1 ; CCCCCCCCC(CCCCCC)C(=O)OCCCCCCCC1(CCCCCCCOC(=O)C(CCCCCCCC)CCCCCCCC)OCC(CCCO)O1]

To a mixture of compound 2 (540 mg, 707.49 μmol, 1 eq.) and compound 2A (283.37 mg, 1.77 mmol, 2.5 eq.) in toluene (20 mL) was added PPTS (53.34 mg, 212.25 μmol, 0.3 eq.), and then the mixture was stirred at 125° C. for 12 hours equipped with Dean-Stark. The mixture was poured into ice-water (w/w=1/1, 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether/Ethyl acetate=8/1 to 5/1) to give compound 3 (483 mg, 78.89% yield) as a colorless oil.

¹HNMR: (400 MHz, CHLOROFORM-d) δ ppm 4.20-3.99 (m, 6H), 3.76-3.64 (m, 2H), 3.51-3.43 (m, 1H), 2.31 (br t, J=5.2 Hz, 2H), 2.17-1.97 (m, 1H), 1.60 (br d, J=6.4 Hz, 17H), 1.49-1.17 (m, 64H), 0.88 (t, J=6.8 Hz, 12H).

### Procedure for Preparation of Compound 4

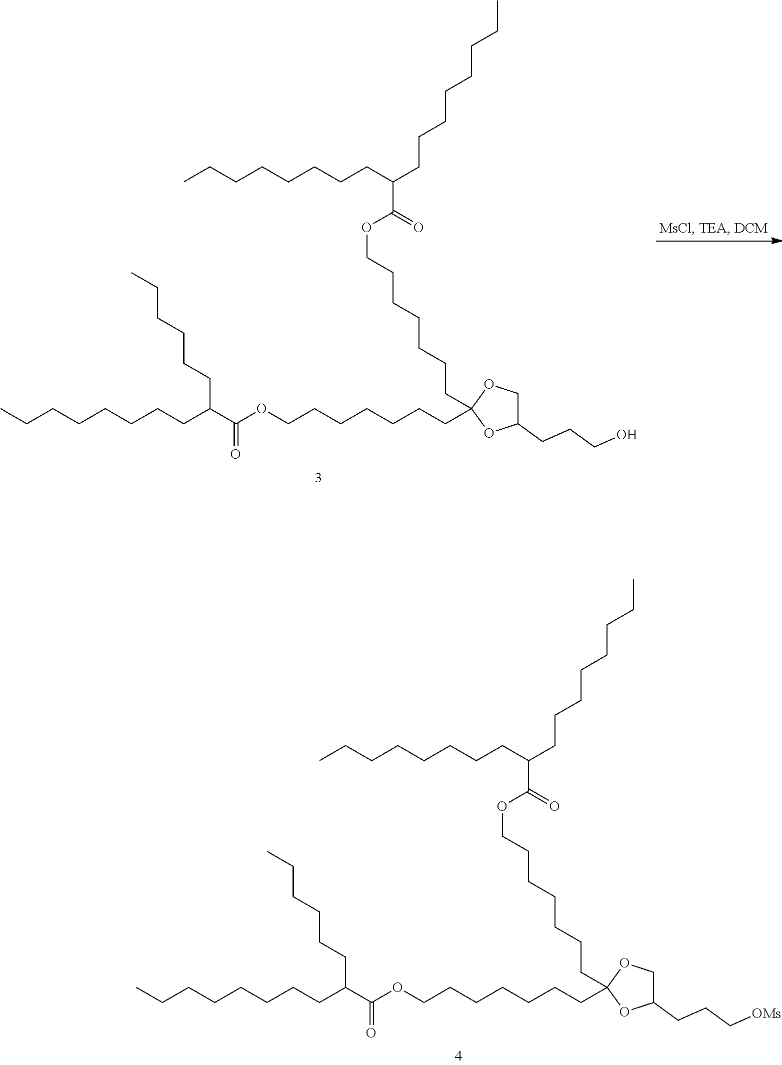 [SMILES: CCCCCCCCC(CCCCCC)C(=O)OCCCCCCCC1(CCCCCCCOC(=O)C(CCCCCCCC)CCCCCCCC)OCC(CCCO)O1 ; CCCCCCCCC(CCCCCC)C(=O)OCCCCCCCC1(CCCCCCCOC(=O)C(CCCCCCCC)CCCCCCCC)OCC(CCCOS(C)(=O)=O)O1]

To a solution of compound 3 (483 mg, 558.13 μmol, 1 eq.) in DCM (5 mL) was added MsCl (255.74 mg, 2.23 mmol, 172.79 μL, 4 eq.) and TEA (282.38 mg, 2.79 mmol, 388.42 μL, 5 eq.). The mixture was stirred at 25° C. for 2 hours. The mixture was poured into ice-water (10 mL), and then extracted with DCM (10 mL*3). The combined organic layers were washed with brine (10 mL), dried over anhydrous Na₂SO₄, filtered and concentrated under reduced pressure to give compound 4 (581 mg, crude) as a yellow oil, which was used directly for next step without further purification.

¹HNMR: (400 MHz, CHLOROFORM-d) δ ppm 4.36-4.20 (m, 2H), 4.15-4.01 (m, 6H), 3.68 (s, 1H), 3.51-3.37 (m, 2H), 3.15 (s, 2H), 3.02 (s, 3H), 2.37-2.23 (m, 2H), 2.01-1.76 (m, 2H), 1.72-1.52 (m, 15H), 1.42 (td, J=7.2, 14.4 Hz, 6H), 1.26 (s, 58H), 0.88 (s, 12H)

### Procedure for Preparation of Lipid 10

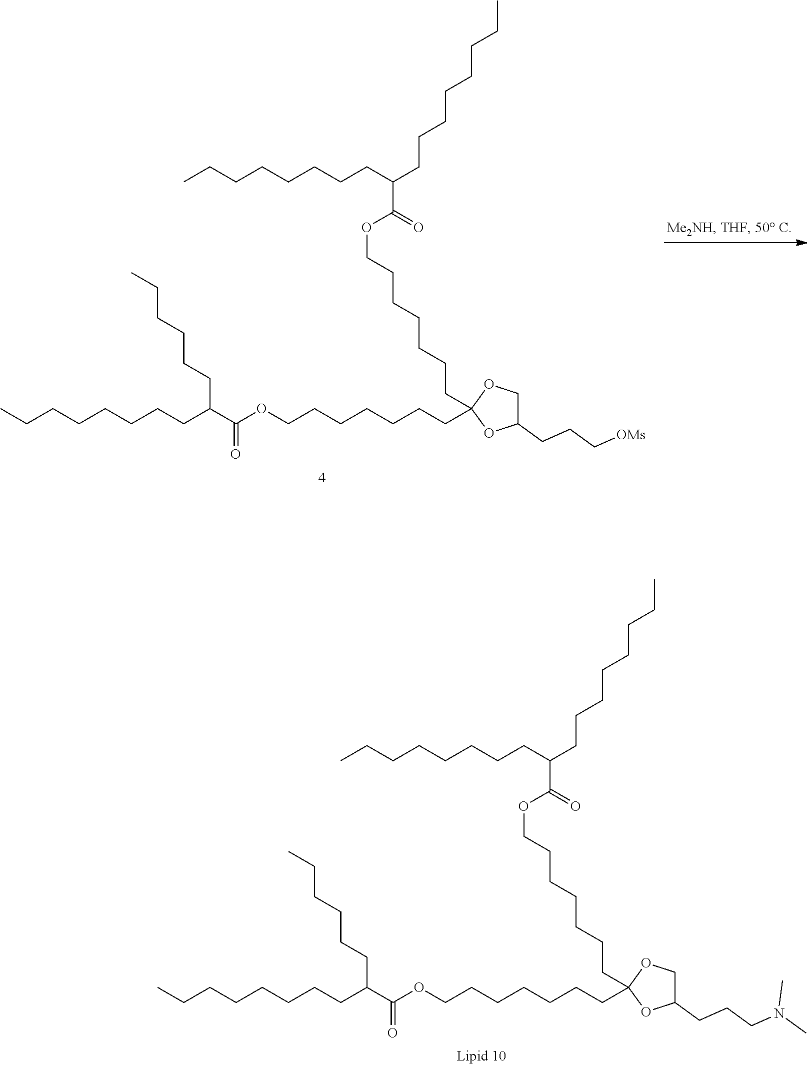 [SMILES: CCCCCCCCC(CCCCCC)C(=O)OCCCCCCCC1(CCCCCCCOC(=O)C(CCCCCCCC)CCCCCCCC)OCC(CCCOS(C)(=O)=O)O1 ; CCCCCCCCC(CCCCCC)C(=O)OCCCCCCCC1(CCCCCCCOC(=O)C(CCCCCCCC)CCCCCCCC)OCC(CCCN(C)C)O1]

To a solution of compound 4 (580 mg, 614.74 μmol, 1 eq.) in THE (10 mL) was added Me₂NH (2 M THF solution, 49.35 mL, 160.56 eq.). The mixture was stirred at 50° C. for 12 hours. The reaction mixture was concentrated under reduced pressure. The residue was purified by column chromatography (SiO₂, PE/EA=10/1 to 5/1, eluent with 0.1% TEA), followed by another column purification (SiO₂, A/B=100/1 to 5/1, phase A: PE:DCM=3:1, phase B: EtOAc, eluent with 0.1% TEA) to give Lipid 10 (360 mg, 65.62% yield) as a colorless oil.

LCMS: m/z 893.1 [M+1]⁺. ¹HNMR: (400 MHz, CHLOROFORM-d) δ ppm 4.00-4.13 (m, 6H), 3.41-3.53 (m, 1H), 2.18-2.37 (m, 10H), 1.51-1.78 (m, 18H), 1.26 (s, 63H), 0.92-0.83 (m, 12H)

### Synthesis of Lipid 11

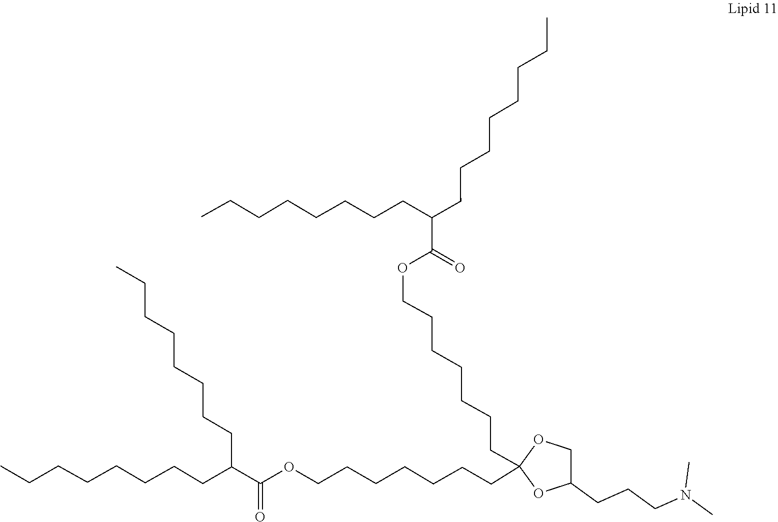 [SMILES: CCCCCCCCC(CCCCCCCC)C(=O)OCCCCCCCC1(CCCCCCCOC(=O)C(CCCCCCCC)CCCCCCCC)OCC(CCCN(C)C)O1]

A scheme for the synthesis of Lipid 11 is provided in Scheme 11 below.

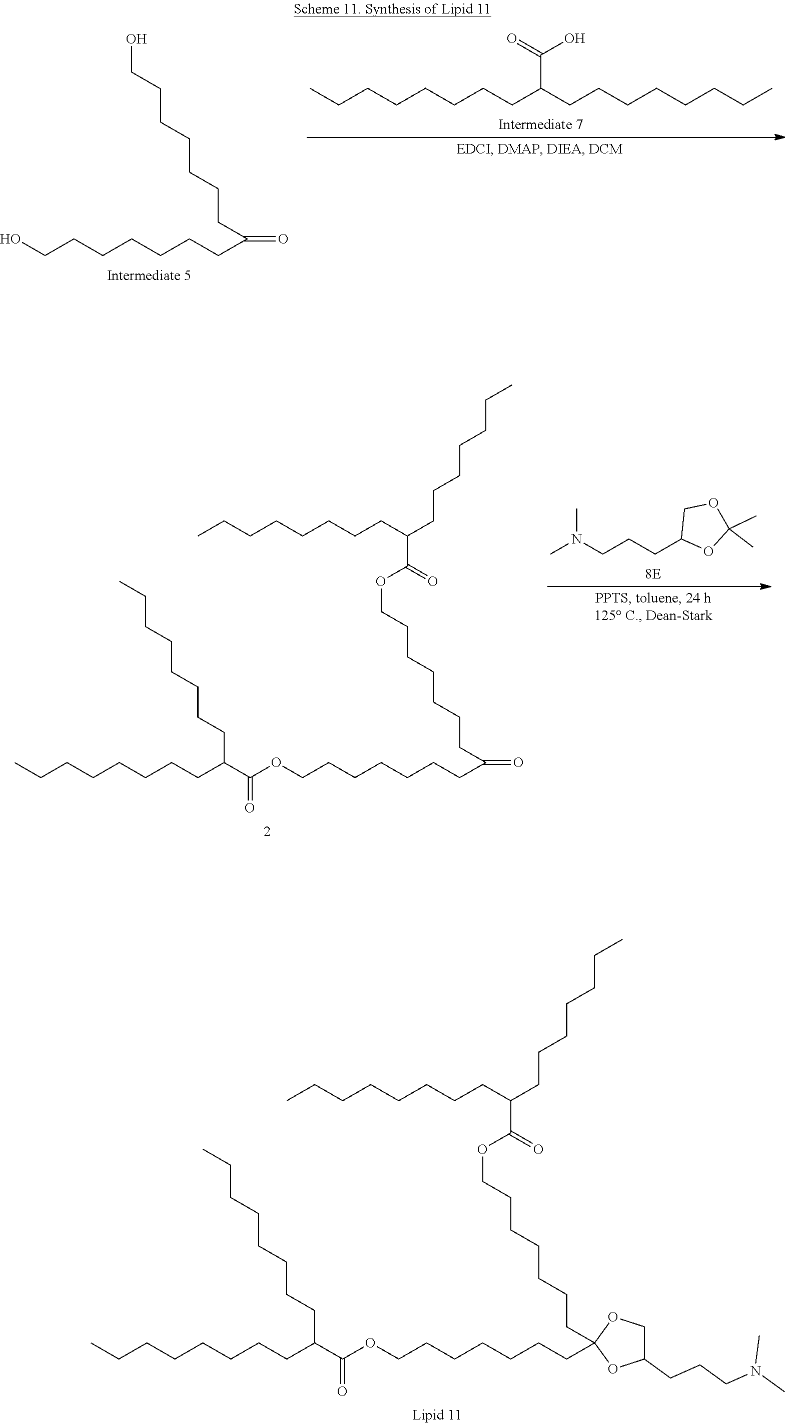

### Procedure for Preparation of Compound 2

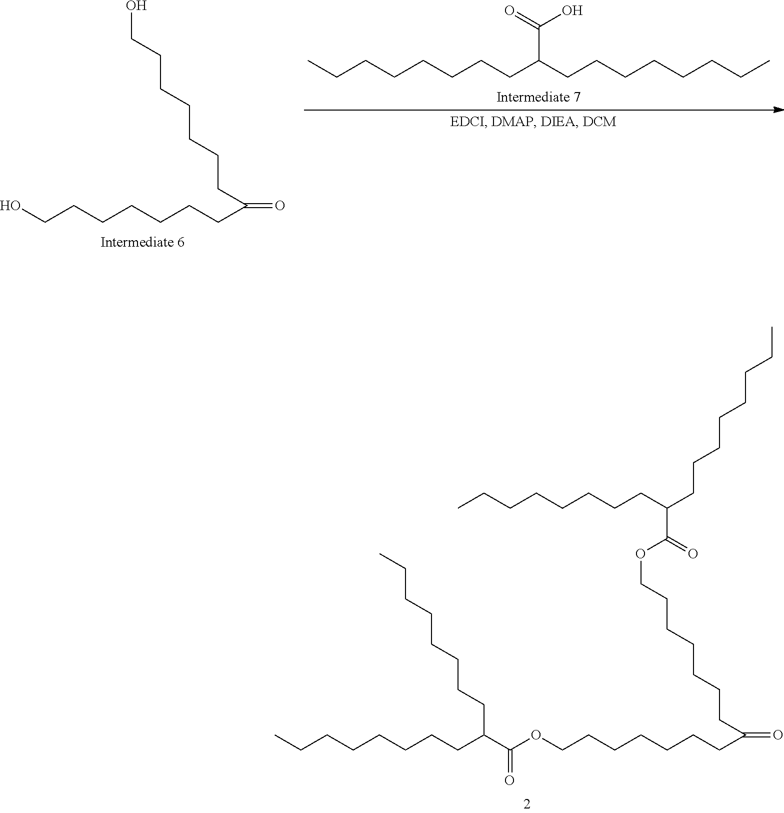

To a solution of intermediate 6 (1 g, 3.87 mmol, 1 eq.) and intermediate 7 (2.31 g, 8.13 mmol, 2.1 eq.) in DCM (10 mL) was added EDCI (2.97 g, 15.48 mmol, 4 eq.), DIEA (4.50 g, 34.83 mmol, 6.07 mL, 9 eq.) and DMAP (472.79 mg, 3.87 mmol, 1 eq.) at 25° C. The mixture was stirred at 40° C. for 12 hours. The reaction mixture was concentrated under reduced pressure. The residue was purified by column chromatography (SiO₂, PE/EA=20/1 to 10/1) to give compound 2 (1.17 g, 38.21% yield) as a white solid.

¹HNMR: (400 MHz, CHLOROFORM-d) δ ppm 4.06 (s, 4H), 2.39 (s, 6H), 1.58 (br s, 14H), 1.48-1.19 (m, 65H), 0.95-0.83 (m, 1H), 0.88 (t, J=6.8 Hz, 11H).

### Procedure for Preparation of Lipid 11

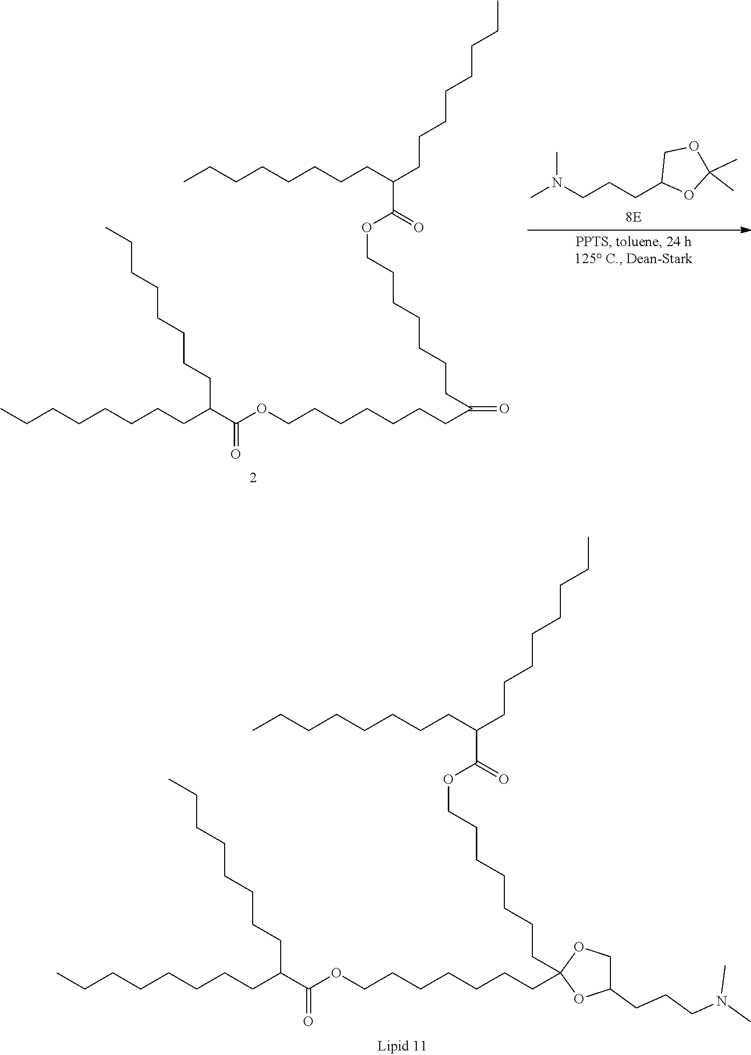

To a solution of compound 2 (1.15 g, 1.45 mmol, 1 eq.) in toluene (20 mL) was added PPTS (182.60 mg, 726.64 μmol, 0.5 eq.), PTSA (82.93 mg, 435.98 μmol, 0.3 eq.) and compound 8E (783.63 mg, 2.18 mmol, 1.5 eq., TsOH salt) at 25° C. The mixture was stirred at 125° C. for 12 hours equipped with Dean-Stark. The reaction mixture was concentrated under reduced pressure. The residue was purified by column chromatography (SiO₂, PE/EA=10/1 to 5/1, eluent with 0.1% TEA), followed by another column purification (SiO₂, A/B=100/1 to 5/1, phase A: PE:DCM=3:1, phase B: EtOAc, eluent with 0.1% TEA) to give Lipid 11 (230 mg, 17.19% yield) as a white oil.

LCMS: m/z 921.0 [M+1]⁺. ¹HNMR: (400 MHz, CHLOROFORM-d) δ ppm 4.07 (br t, J=6.8 Hz, 6H), 3.51-3.41 (m, 1H), 2.50-2.21 (m, 8H), 1.67-1.53 (m, 20H), 1.26 (s, 66H), 0.93-0.82 (m, 12H).

### Synthesis of Lipid 12

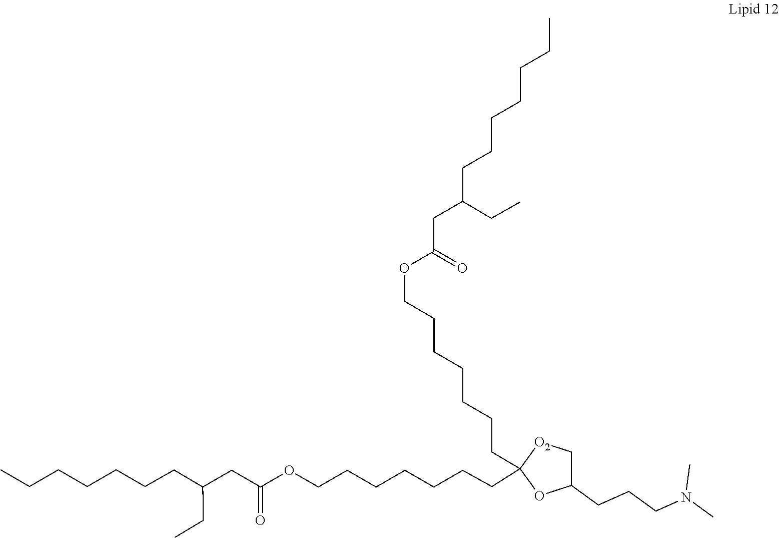

A scheme for the synthesis of Lipid 12 is provided in Scheme 12 below.

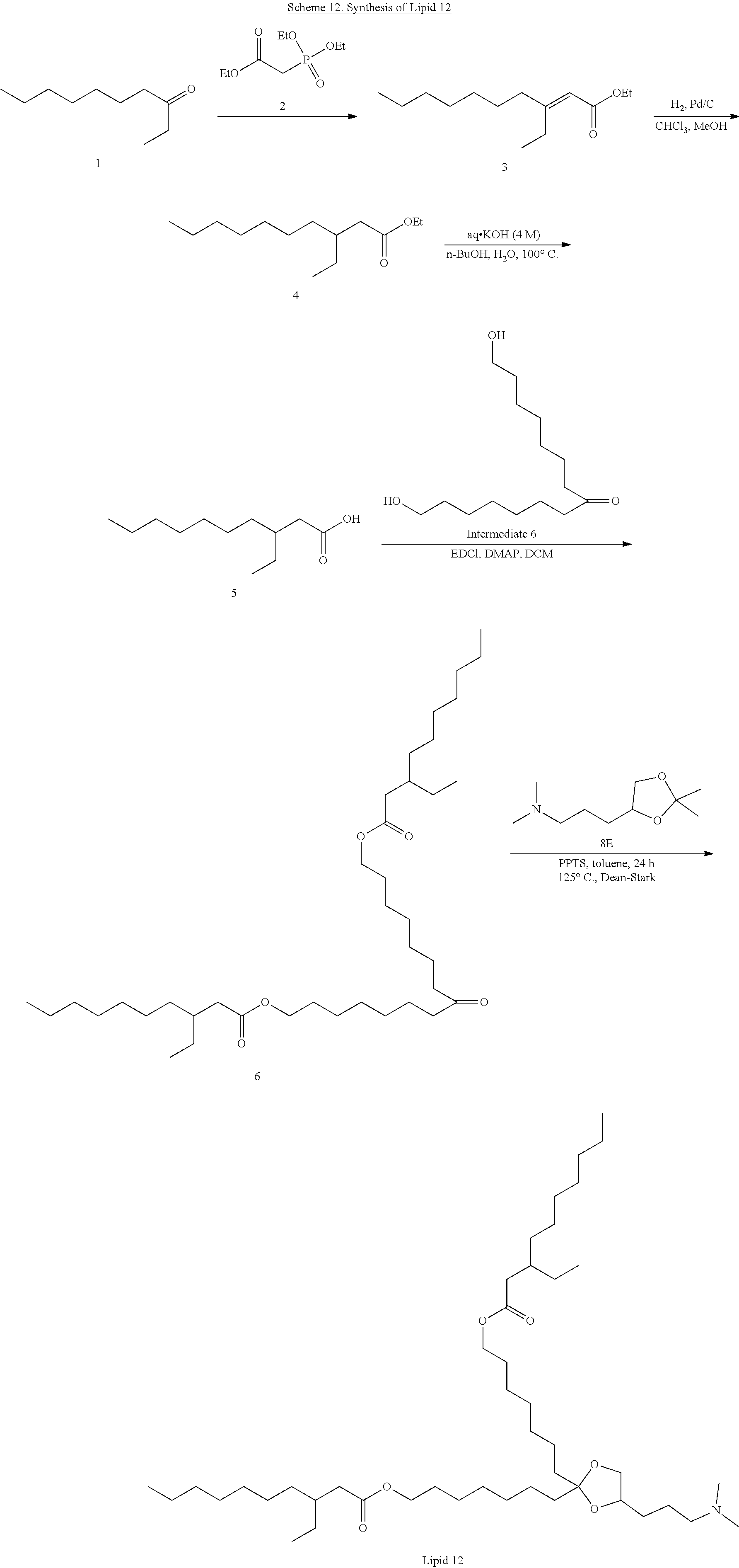

### Procedure for Preparation of Compound 3

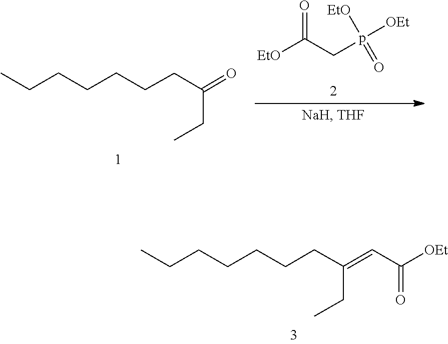

To a solution of compound 1 (2.79 g, 17.84 mmol, 0.8 eq.) and compound 2 (5 g, 22.30 mmol, 4.42 mL, 1 eq.) in THF (50 mL) was added NaH (981.31 mg, 24.53 mmol, 60% purity, 1.1 eq.) at 0° C. The mixture was stirred at 25° C. for 2 hours. The residue was poured into ice-water (w/w=1/1, 20 mL) and stirred for 2 mins. The aqueous phase was extracted with ethyl acetate (30 mL*3). The combined organic phases were washed with brine (40 mL), dried over anhydrous Na₂SO₄, filtered and concentrated under vacuum. The residue was purified by column chromatography (SiO₂, Petroleum ether/Ethyl acetate=I/O to 50/1) to give compound 3 (3.8 g, 75.27% yield) as a yellow oil.

¹HNMR: (400 MHz, CHLOROFORM-d) δ ppm 5.64 (d, J=4.8 Hz, 1H), 4.18 (dd, J=0.8, 7.2 Hz, 2H), 2.73-2.59 (m, 2H), 2.25-2.15 (m, 2H), 1.53-1.44 (m, 2H), 1.33-1.27 (m, 11H), 1.10 (dt, J=1.6, 7.5 Hz, 3H), 0.94-0.91 (m, 3H).

### Procedure for Preparation of Compound 4

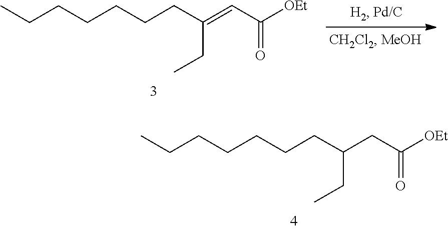 [SMILES: CCCCCCC/C(=C/C(=O)OCC)CC ; CCCCCCCC(CC)CC(=O)OCC]

To a solution of compound 3 (2.8 g, 12.37 mmol, 1 eq.) in a mixed solvents of MeOH (18 mL) and CH₂Cl₂ (2 mL) was added Pd/C (1.5 g, 10% on carbon) under N₂ atmosphere. The suspension was degassed under vacuum and purged with Hz for several times. The mixture was stirred under H₂ (15 Psi) atmosphere at 25° C. for 2 hours. The reaction mixture was filtered and the filtrate was concentrated under reduced pressure to give compound 4 (2.2 g, crude) as a yellow oil, which was used directly for next step without further purification.

### Procedure for Preparation of Compound 5

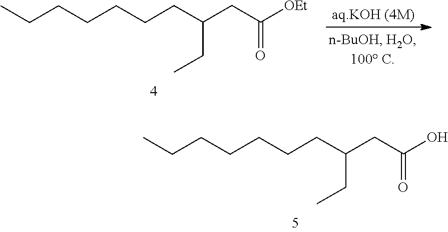 [SMILES: CCCCCCCC(CC)CC(=O)OCC ; CCCCCCCC(CC)CC(=O)O]

To a solution of compound 4 (3 g, 13.14 mmol, 1 eq.) in EtOH (20 mL) was added KOH (4 M aqueous solution, 22.99 mL, 7 eq.) at 25° C. The mixture was stirred at 80° C. for 12 hours. The reaction mixture was quenched by addition of HCl (2 M aqueous solution, 10 mL) at 0° C., and then extracted with EtOAc (40 mL*3). The combined organic layers were washed with brine (60 mL), dried over anhydrous Na₂SO₄, filtered and concentrated under reduced pressure. The residue was purified by column chromatography (SiO₂, Petroleum ether/Ethyl acetate=I/O to 20/1) to give compound 5 (2.5 g, 95% yield) as a yellow oil.

¹HNMR: (400 MHz, CHLOROFORM-d) δ ppm 2.30 (d, J=7.2 Hz, 2H), 1.90-1.77 (m, 1H), 1.46-1.24 (m, 14H), 0.97-0.85 (m, 6H).

### Procedure for Preparation of Compound 6

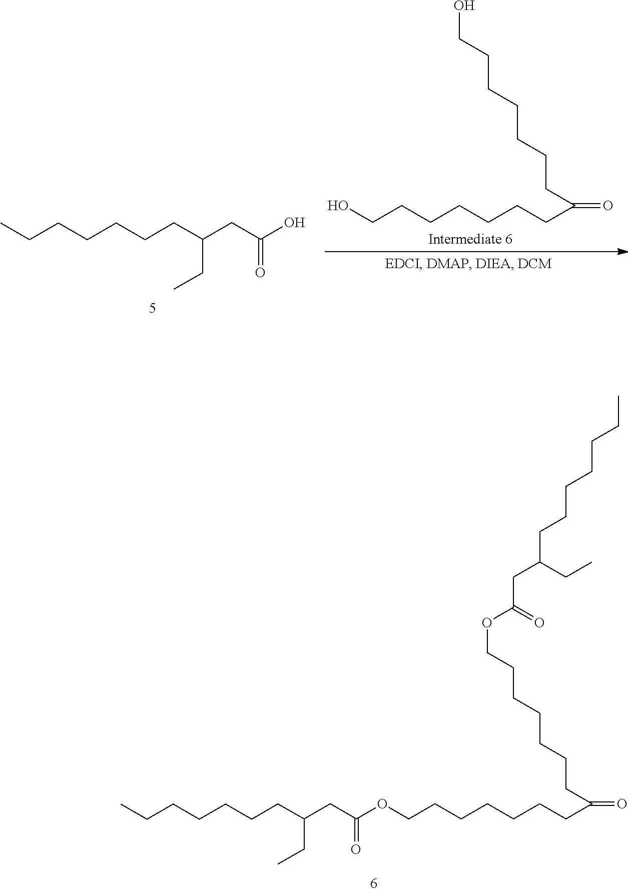 [SMILES: O=C(CCCCCCCO)CCCCCCCO ; CCCCCCCC(CC)CC(=O)O ; CCCCCCCC(CC)CC(=O)OCCCCCCCC(=O)CCCCCCCOC(=O)CC(CC)CCCCCCC]

To a solution of intermediate 6 (1 g, 3.87 mmol, 1 eq.) and compound 5 (1.55 g, 7.74 mmol, 2 eq.) in DCM (10 mL) was added DIEA (4.50 g, 34.83 mmol, 6.07 mL, 9 eq.), DMAP (472.79 mg, 3.87 mmol, 1 eq.) and EDCI (2.97 g, 15.48 mmol, 4 eq.) at 25° C. The mixture was stirred at 40° C. for 12 hours. The mixture was poured into ice-water (w/w=1/1, 20 mL) and stirred for 2 mins. The aqueous phase was extracted with ethyl acetate (30 mL*3). The combined organic phases were washed with brine (40 mL), dried over anhydrous Na₂SO₄, filtered and concentrated under vacuum. The residue was purified by column chromatography (SiO₂, Petroleum ether/Ethyl acetate=I/O to 20/1) to give compound 6 (1.01 g, 41.89% yield) as a yellow oil.

¹HNMR: (400 MHz, CHLOROFORM-d) δ ppm 4.08 (t, J=6.4 Hz, 4H), 2.42 (t, J=7.2 Hz, 4H), 2.26 (d, J=7.2 Hz, 4H), 1.88-1.77 (m, 2H), 1.69-1.59 (m, 8H), 1.44-1.24 (m, 40H), 0.98-0.86 (m, 12H).

### Procedure for Preparation of Lipid 12

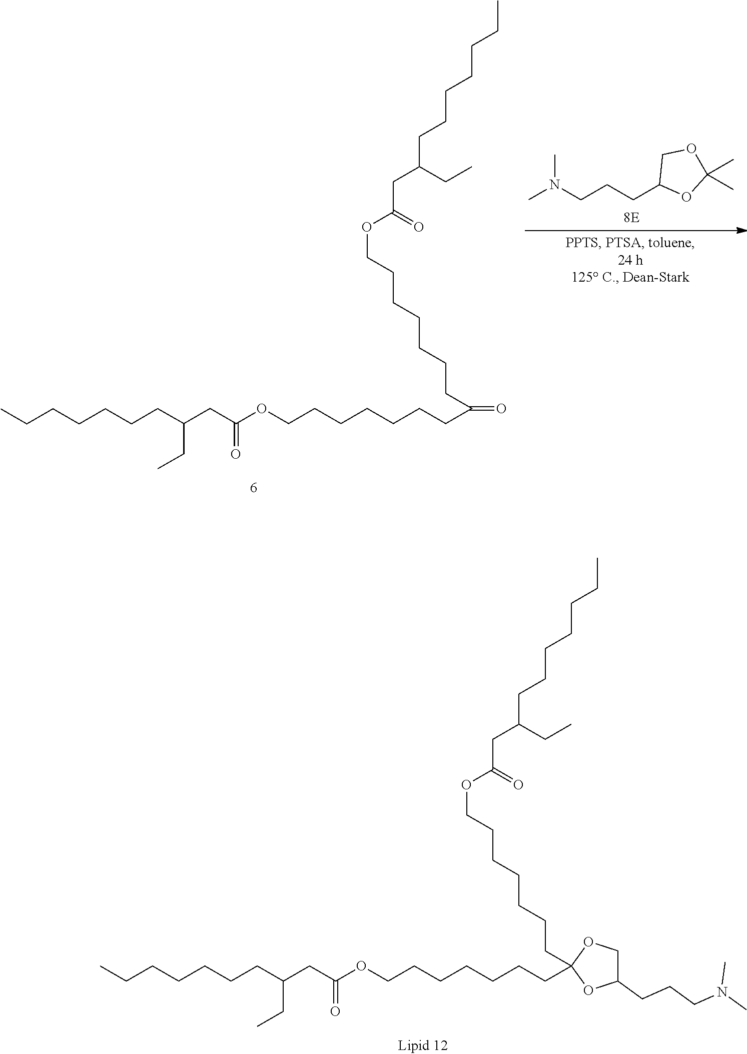

To a solution of compound 6 (1 g, 1.61 mmol, 1 eq.) and compound 8E (1.15 g, 3.21 mmol, 2 eq., TsOH salt) in toluene (10 mL) was added PPTS (201.69 mg, 802.57 μmol, 0.5 eq.) and PTSA (91.60 mg, 481.54 μmol, 0.3 eq.) at 25° C. The mixture was stirred at 125° C. for 12 hours equipped with Dean-Stark. The mixture was poured into sat. NaHCO₃ (aq., 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether (1% TEA)/Ethyl acetate (1% TEA)=15/1 to 3/1), followed by another column purification (SiO₂, A/B=100/1 to 5/1, phase A: PE:DCM=3:1, phase B: EtOAc, eluent with 0.1% TEA) to give Lipid 12 (450 mg, 36.90% yield, 99% purity) as a yellow oil.

LCMS: m/z 752.9 [M+1]⁺. ¹HNMR: (400 MHz, CHLOROFORM-d) δ ppm 4.07 (t, J=7.2 Hz, 6H), 3.47 (s, 1H), 2.47-2.18 (m, 12H), 1.86-1.77 (m, 2H), 1.69-1.53 (m, 12H), 1.40-1.24 (m, 44H), 0.95-0.85 (m, 12H).

### Synthesis of Lipid 13 and Lipid 14

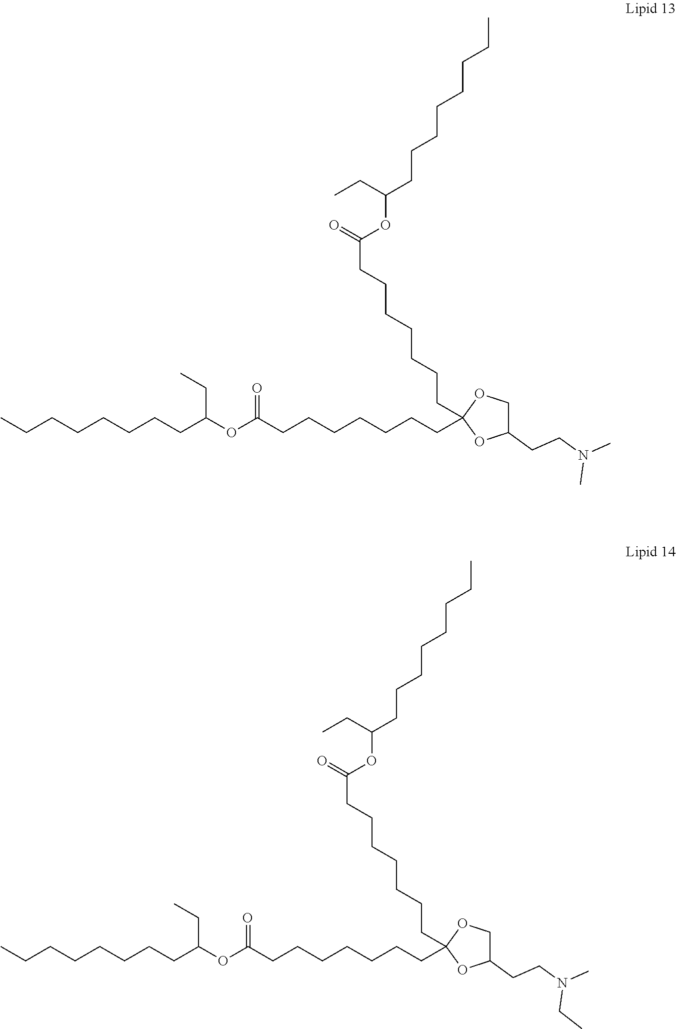

A scheme for the synthesis of Lipid 13 and Lipid 14 is provided in Scheme 13 below.

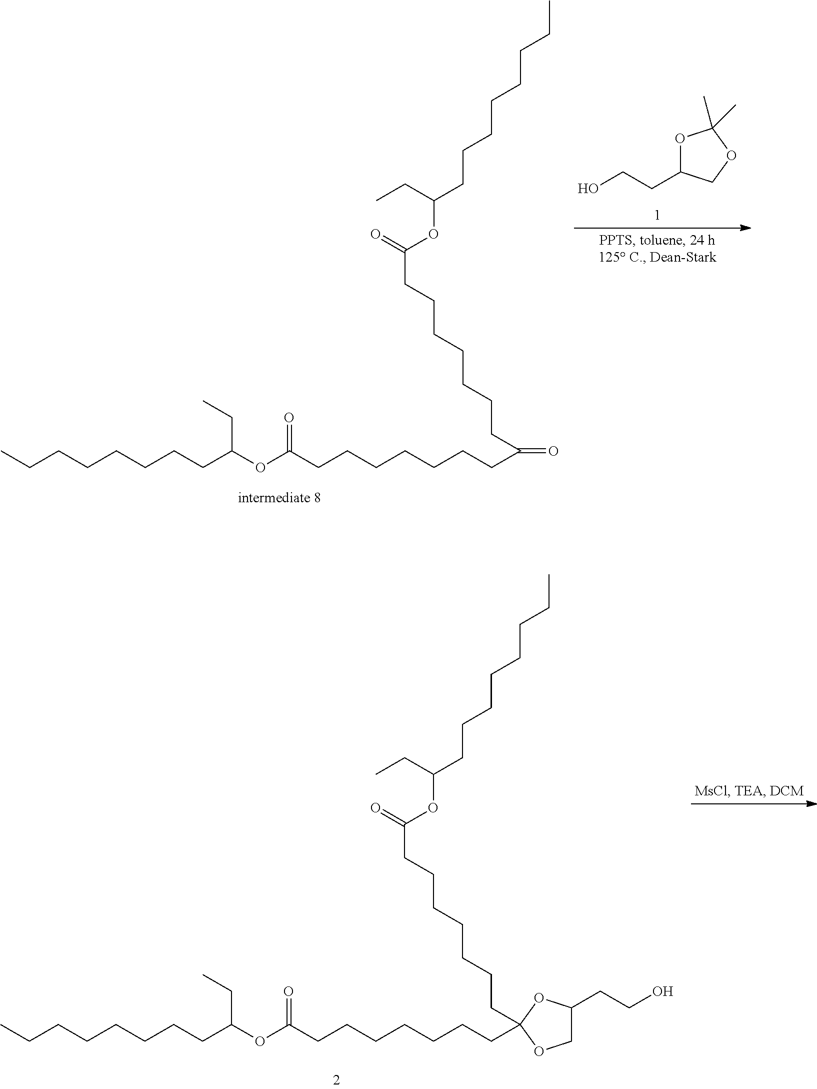

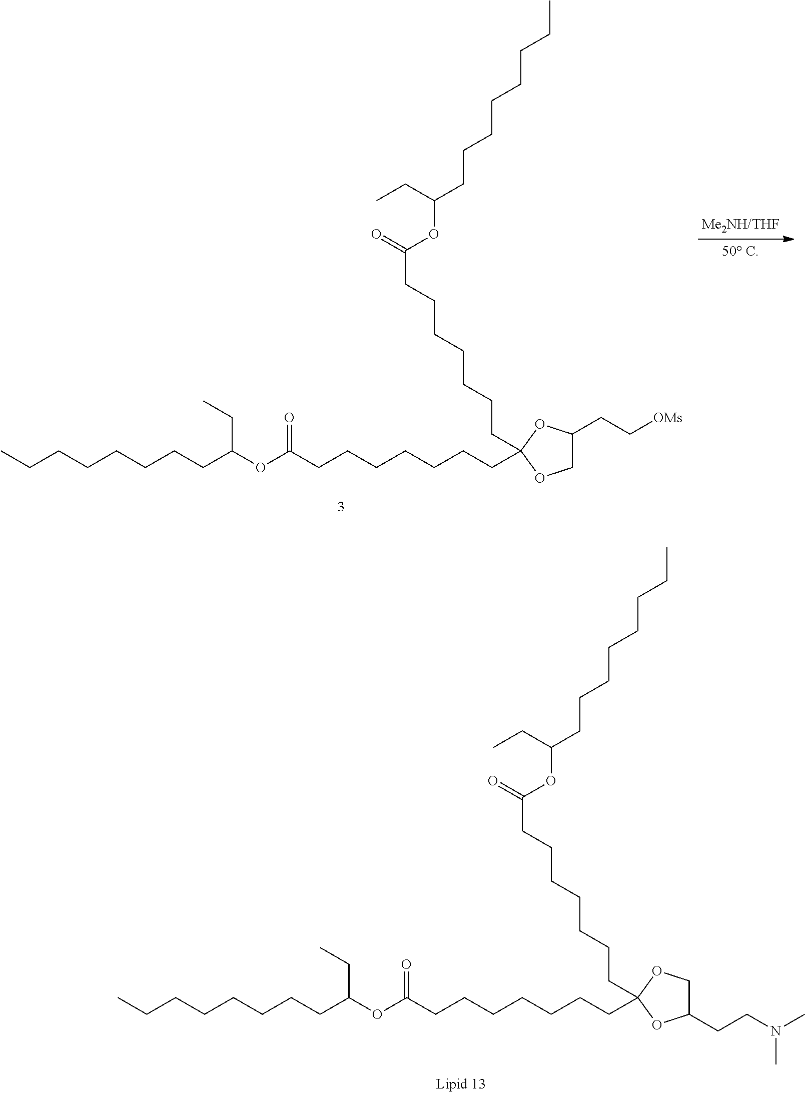 [SMILES: CCCCCCCCC(CC)OC(=O)CCCCCCCC1(CCCCCCCC(=O)OC(CC)CCCCCCCC)OCC(CCOS(C)(=O)=O)O1 ; CCCCCCCCC(CC)OC(=O)CCCCCCCC1(CCCCCCCC(=O)OC(CC)CCCCCCCC)OCC(CCN(C)C)O1]

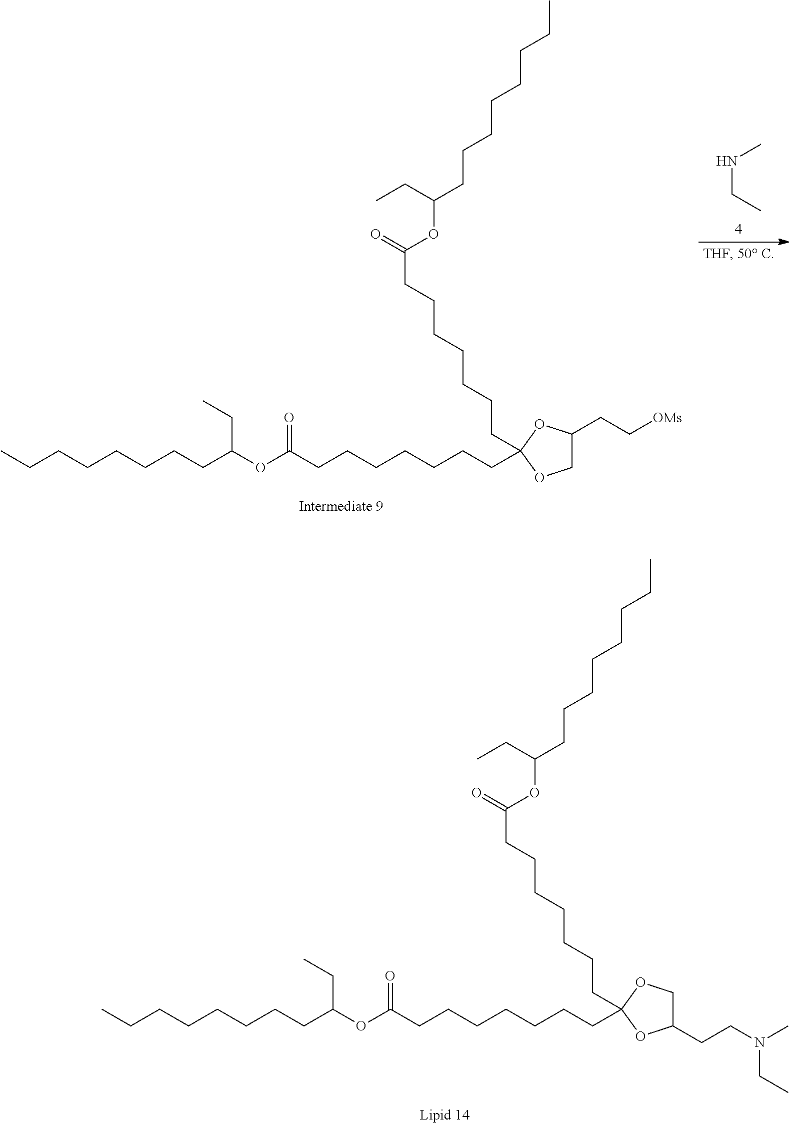

### Procedure for Preparation of Compound 2

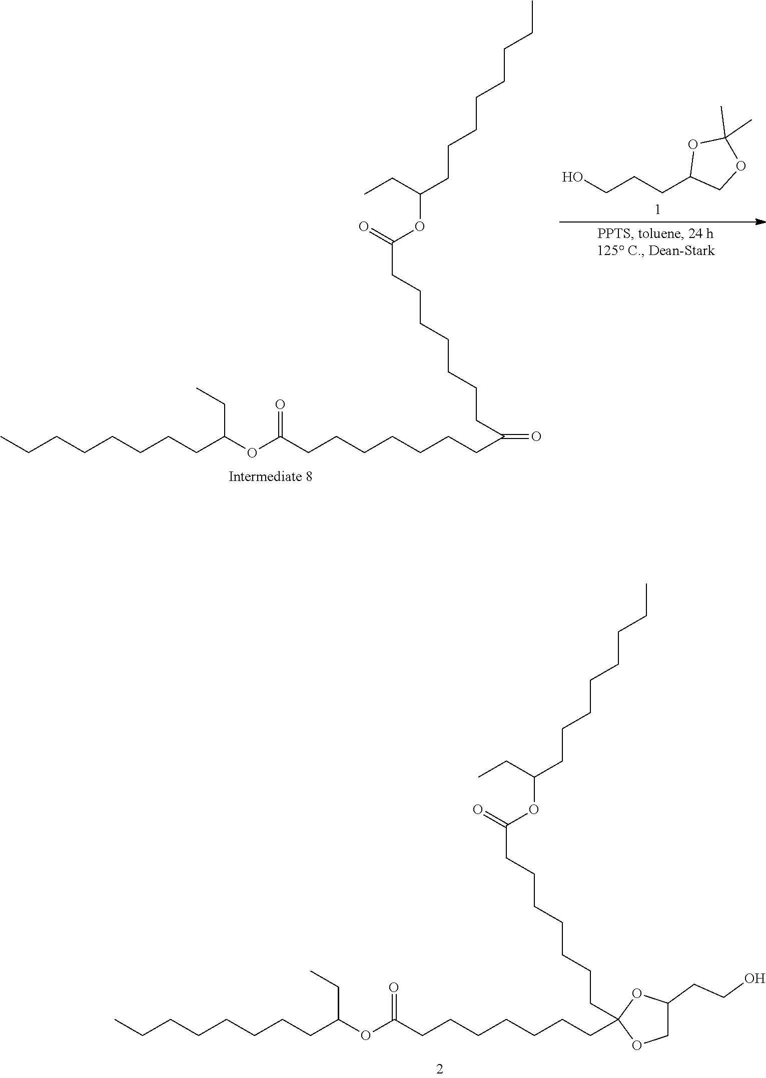 [SMILES: CCCCCCCCC(CC)OC(=O)CCCCCCCC(=O)CCCCCCCC(=O)OC(CC)CCCCCCCC ; CC1(C)OCC(CCCO)O1 ; CCCCCCCCC(CC)OC(=O)CCCCCCCC1(CCCCCCCC(=O)OC(CC)CCCCCCCC)OCC(CCO)O1]

To a solution of intermediate 8 (1 g, 1.61 mmol, 1 eq.) and compound 1 (586.61 mg, 4.01 mmol, 568.97 μL, 2.5 eq.) in toluene (40 mL) was added and PPTS (121.01 mg, 481.54 μmol, 0.3 eq.) at 25° C. and then the mixture was stirred at 125° C. for 12 hours equipped with Dean-Stark. The mixture was poured into sat. NaHCO₃ (aq., 50 mL), and extracted with EtOAc (50 mL*3). The combined organic layers were washed with brine (10 mL), dried over anhydrous Na₂SO₄, filtered and concentrated under reduced pressure. The residue was purified by column chromatography (SiO₂, Petroleum ether/Ethyl acetate=20/1˜10/1˜5/1) to give compound 2 (830 mg, 65.44% yield, >90% purity) as yellow oil.

¹HNMR: (400 MHz, CHLOROFORM-d) δ ppm 4.82 (quin, J=6.0 Hz, 2H), 4.32-4.19 (m, 1H), 4.12-4.05 (m, 1H), 3.81 (br t, J=5.2 Hz, 2H), 3.53 (t, J=8.0 Hz, 1H), 2.29 (t, J=7.6 Hz, 4H), 1.82 (q, J=5.6 Hz, 2H), 1.73-1.47 (m, 17H), 1.43-1.15 (m, 42H), 0.97-0.77 (m, 12H).

### Procedure for Preparation of Compound 3

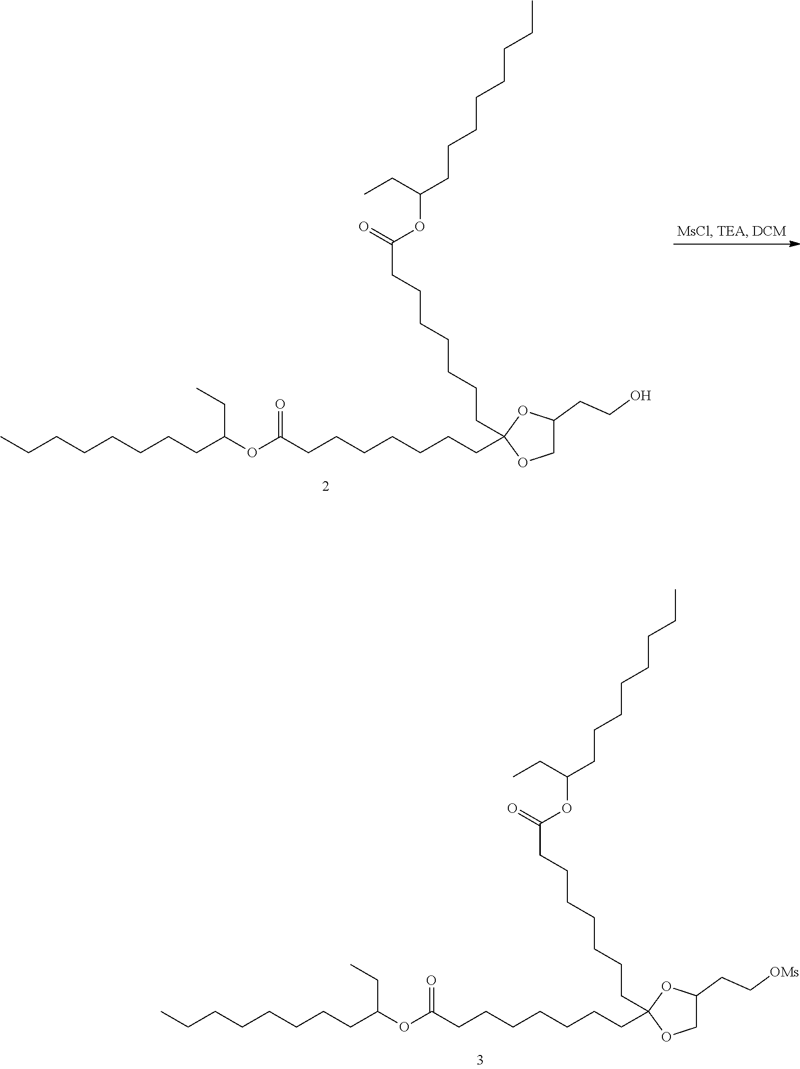 [SMILES: CCCCCCCCC(CC)OC(=O)CCCCCCCC1(CCCCCCCC(=O)OC(CC)CCCCCCCC)OCC(CCO)O1 ; CCCCCCCCC(CC)OC(=O)CCCCCCCC1(CCCCCCCC(=O)OC(CC)CCCCCCCC)OCC(CCOS(C)(=O)=O)O1]

To a mixture of compound 2 (820 mg, 1.15 mmol, 1 eq.) and TEA (583.42 mg, 5.77 mmol, 802.51 μL, 5 eq.) in DCM (20 mL) was added MsCl (396.28 mg, 3.46 mmol, 267.76 μL, 3 eq.) at 5° C., and then the mixture was stirred at 25° C. for 12 hours. The mixture was poured into ice-water (20 mL) and extracted with DCM (10 mL*3). The combined organic layers were washed with brine (10 mL), dried over anhydrous Na₂SO₄, filtered and concentrated under reduced pressure to give compound 4 (820 mg, crude) as a yellow oil, which was used directly for next step without further purification.

¹HNMR: (400 MHz, CHLOROFORM-d) δ ppm 4.89-4.74 (m, 2H), 4.45-4.30 (m, 2H), 4.22-4.14 (m, 1H), 4.12-4.06 (m, 1H), 3.53 (t, J=7.6 Hz, 1H), 3.03 (s, 3H), 2.29 (t, J=7.6 Hz, 4H), 2.09-1.91 (m, 2H), 1.67-1.46 (m, 18H), 1.38-1.20 (m, 42H), 0.97-0.79 (m, 12H).

### Procedure for Preparation of Lipid 13

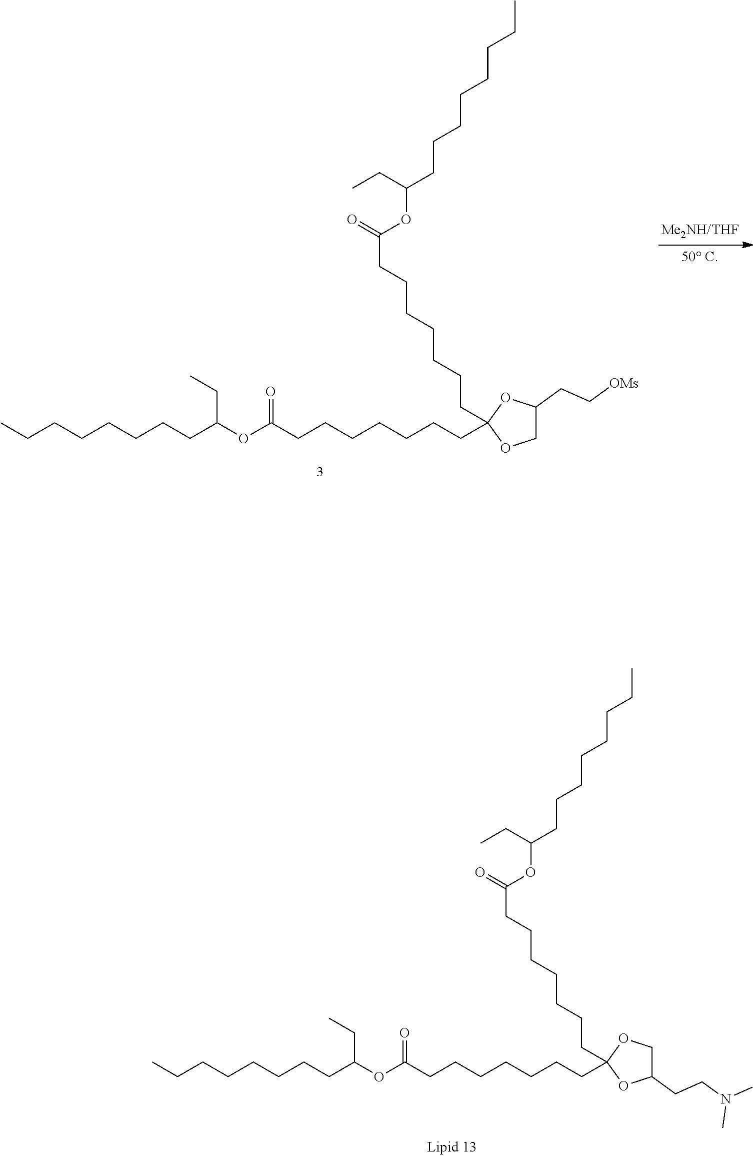 [SMILES: CCCCCCCCC(CC)OC(=O)CCCCCCCC1(CCCCCCCC(=O)OC(CC)CCCCCCCC)OCC(CCOS(C)(=O)=O)O1 ; CCCCCCCCC(CC)OC(=O)CCCCCCCC1(CCCCCCCC(=O)OC(CC)CCCCCCCC)OCC(CCN(C)C)O1]

A mixture of compound 3 (400 mg, 506.85 μmol, 1 eq.) in Me₂NH (2 M THE solution, 5 mL) was stirred at 50° C. for 16 hours. The reaction mixture was concentrated under reduced pressure. The residue was purified by column chromatography (SiO₂, A/B=100/1 to 10/1, phase A: PE:DCM=3:1, phase B: EtOAc, eluent with 0.1% TEA)₃ times to give Lipid 13 (250 mg, 66.15% yield, 99% purity) as a yellow oil.

LCMS: m/z 738.8 [M+1]⁺. ¹HNMR: (400 MHz, CHLOROFORM-d) δ ppm 4.84-4.76 (m, 2H), 4.11-4.01 (m, 2H), 3.50-3.43 (m, 1H), 2.43-2.24 (m, 7H), 2.22 (s, 6H), 1.88-1.73 (m, 1H), 1.71-1.62 (m, 3H), 1.60-1.47 (m, 14H), 1.37-1.22 (m, 41H), 0.90-0.83 (m, 12H).

### Procedure for Preparation of Lipid 14

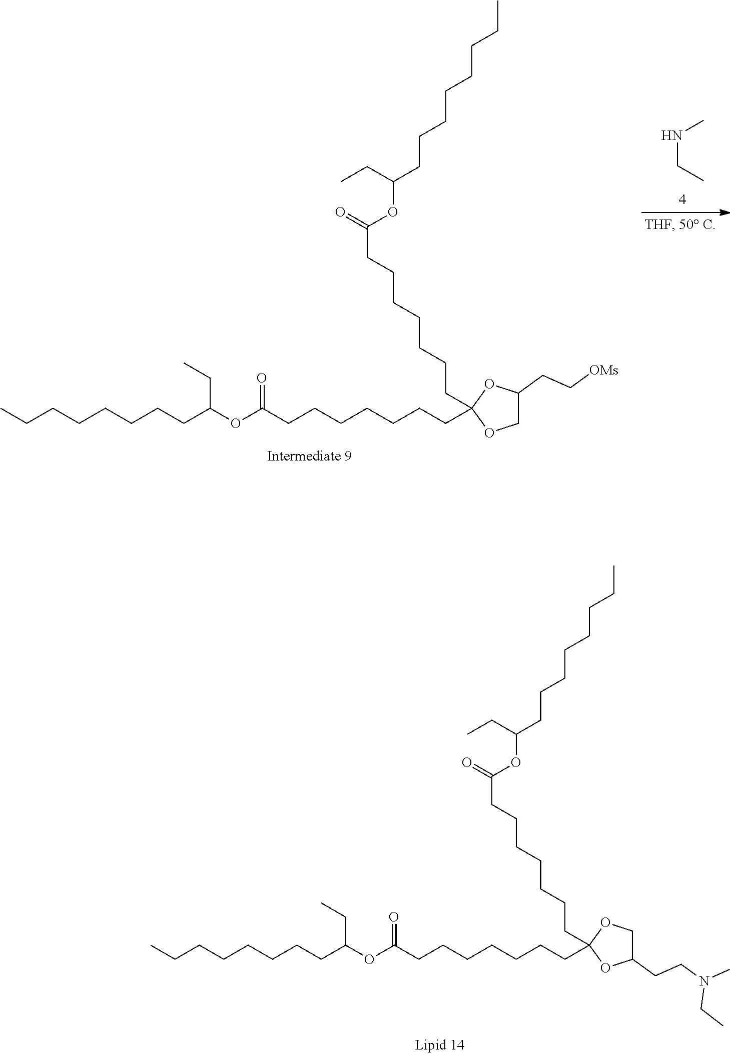

To a solution of intermediate 9 (400 mg, 506.85 μmol, 1 eq.) in THF (2 mL) was added compound 4 (299.60 mg, 5.07 mmol, 435.46 μL, 10 eq.) at 25° C., and then the mixture was stirred at 50° C. for 30 mins. The mixture was poured into sat. NaHCO₃ (aq., 50 mL) and extracted with EtOAc (20 mL*3). The combined organic layers were washed with brine (10 mL), dried over anhydrous Na₂SO₄, filtered and concentrated under reduced pressure. The residue was purified by flash silica gel chromatography (ISCO®; 20 g SepaFlash® Silica Flash Column, Eluent of 0˜20%˜70%, Ethyl acetate (1% TEA)/Petroleum ethergradient (1% TEA), @ 60 mL/min) twice to give Lipid 14 (260 mg, 67.51% yield, 99% purity) as a colorless oil.

LCMS: m/z 752.8 [M+1]⁺. ¹HNMR: (400 MHz, CHLOROFORM-d) δ ppm 4.88-4.76 (m, 2H), 4.13-4.01 (m, 2H), 3.53-3.44 (m, 1H), 2.58-2.36 (m, 4H), 2.31-2.24 (m, 6H), 1.88-1.72 (m, 2H), 1.70-1.44 (m, 18H), 1.40-1.20 (m, 40H), 1.09 (br t, J=7.2 Hz, 3H), 0.92-0.84 (m, 12H).

### Synthesis of Lipid 15

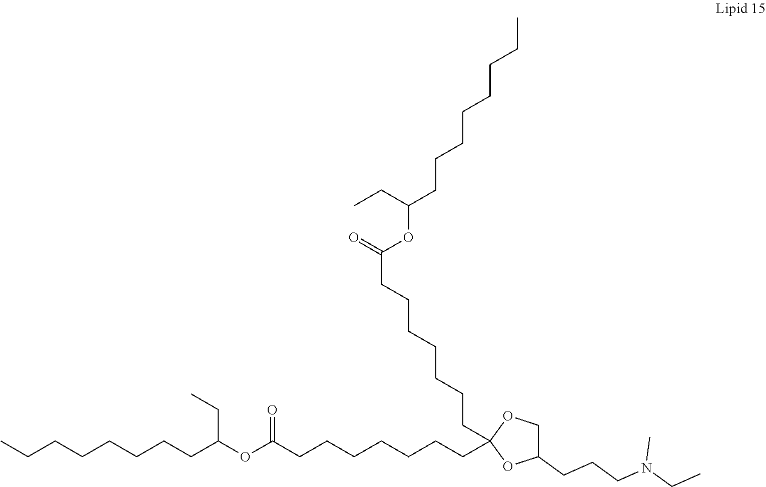 [SMILES: CCCCCCCCC(CC)OC(=O)CCCCCCCC1(CCCCCCCC(=O)OC(CC)CCCCCCCC)OCC(CCCN(C)CC)O1]

A scheme for the synthesis of Lipid 15 is provided in Scheme 14 below.

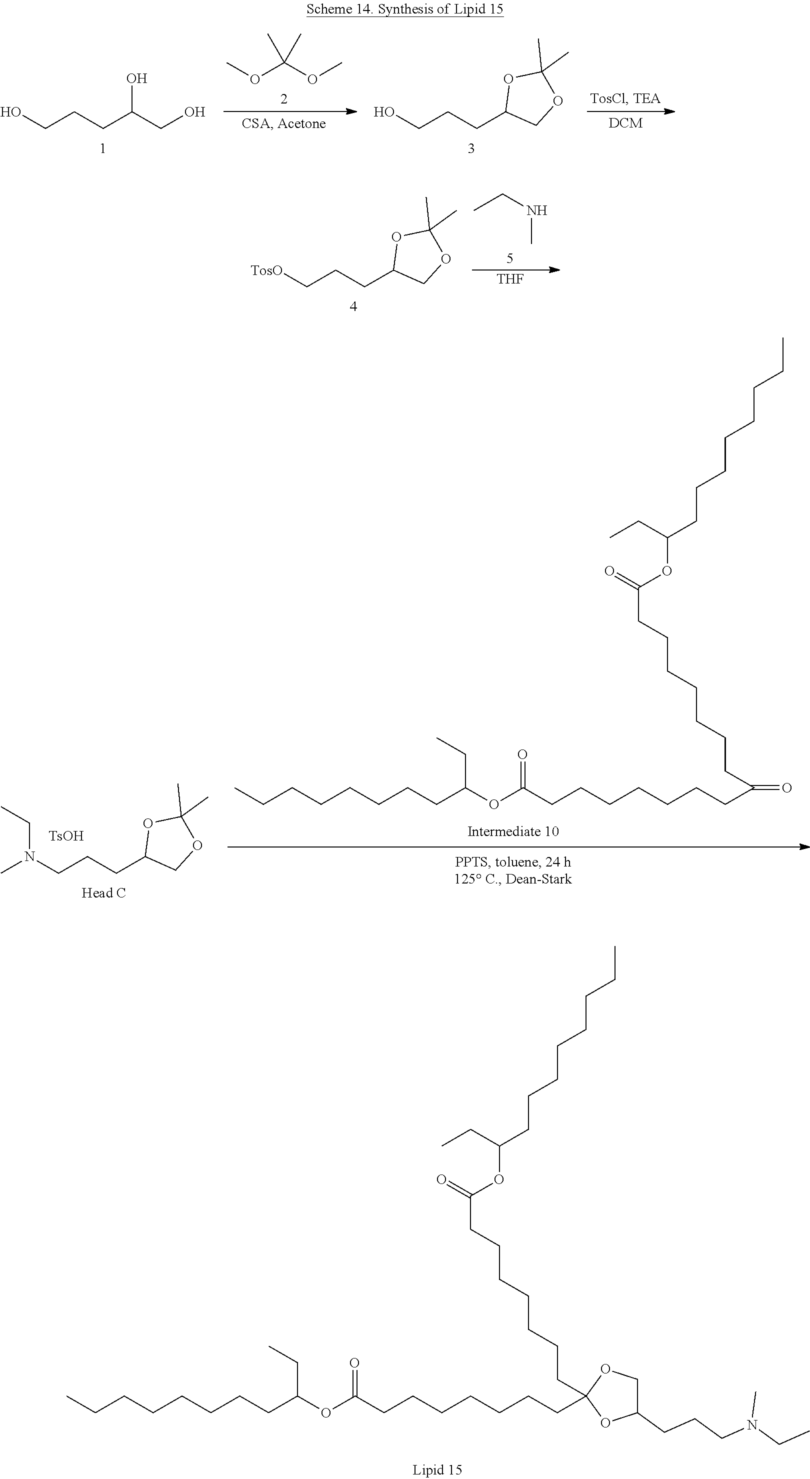

### Procedure for Preparation of Compound 3

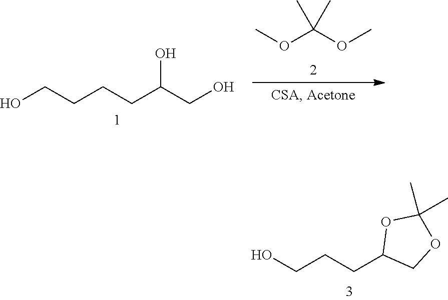

To a mixture of compound 1 (2 g, 16.65 mmol, 1 eq.) and compound 2 (3.47 g, 33.29 mmol, 4.08 mL, 2 eq.) in acetone (60 mL) was added CSA (386.69 mg, 1.66 mmol, 0.1 eq.), and then the mixture was stirred at 20° C. for 12 hours. The reaction mixture was directly concentrated under reduced pressure. The residue was purified by column chromatography (SiO₂, Petroleum ether/Ethyl acetate=10/1 to 1/1) to give compound 3 (2.1 g, 78.74% yield) as yellow oil.

¹HNMR: (400 MHz, CHLOROFORM-d) δ ppm 4.01-4.18 (m, 2H), 3.64-3.79 (m, 2H), 3.51-3.59 (m, 1H), 1.61-1.77 (m, 4H), 1.34-1.46 (m, 6H).

### Procedure for Preparation of Compound 4

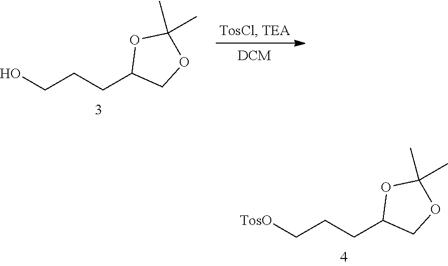

To a mixture of compound 3 (2.1 g, 13.11 mmol, 1 eq.) and TEA (3.98 g, 39.32 mmol, 5.47 mL, 3 eq.) in DCM (20 mL) was added TosCl (3.75 g, 19.66 mmol, 1.5 eq.), and then the mixture was stirred at 20° C. for 12 hours. The reaction mixture was concentrated under vacuum. The residue was purified by column chromatography (SiO₂, Petroleum ether/Ethyl acetate=10/1 to 1/1) to give compound 4 (3.5 g, 84.93% yield) as a yellow oil.

¹HNMR: (400 MHz, CHLOROFORM-d) δ ppm 7.75-7.85 (m, 2H), 7.31-7.39 (m, 2H), 3.97-4.16 (m, 4H), 3.44-3.54 (m, 1H), 2.46 (s, 3H), 1.66-1.91 (m, 2,H), 1.53-1.63 (m, 2H), 1.29-1.40 (m, 6H).

### Procedure for Preparation of Head C

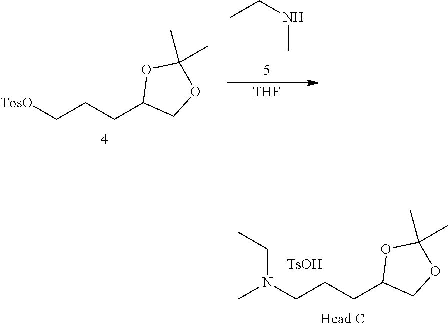

A mixture of compound 4 (3.5 g, 11.13 mmol, 1 eq.) and N-methylethanamine (1.97 g, 33.40 mmol, 2.87 mL, 3 eq.) in THF (7 mL) was stirred at 20° C. for 12 hours. The reaction mixture was concentrated under vacuum and then co-evaporated with toluene for three times to give Head C (3.8 g, 91.39% yield, TsOH salt) as a yellow solid.

¹HNMR: (400 MHz, CHLOROFORM-d) δ ppm 7.70-7.82 (m, 2H), 7.13-7.23 (m, 2H), 3.95-4.08 (m, 2H), 3.42-3.55 (m, 1H), 2.95-3.14 (m, 4H), 2.76 (s, 3H), 2.35 (s, 3H), 1.74-1.96 (m, 2H), 1.46-1.68 (m, 2H), 1.29-1.40 (m, 9H)

### Procedure for Preparation of Lipid 15

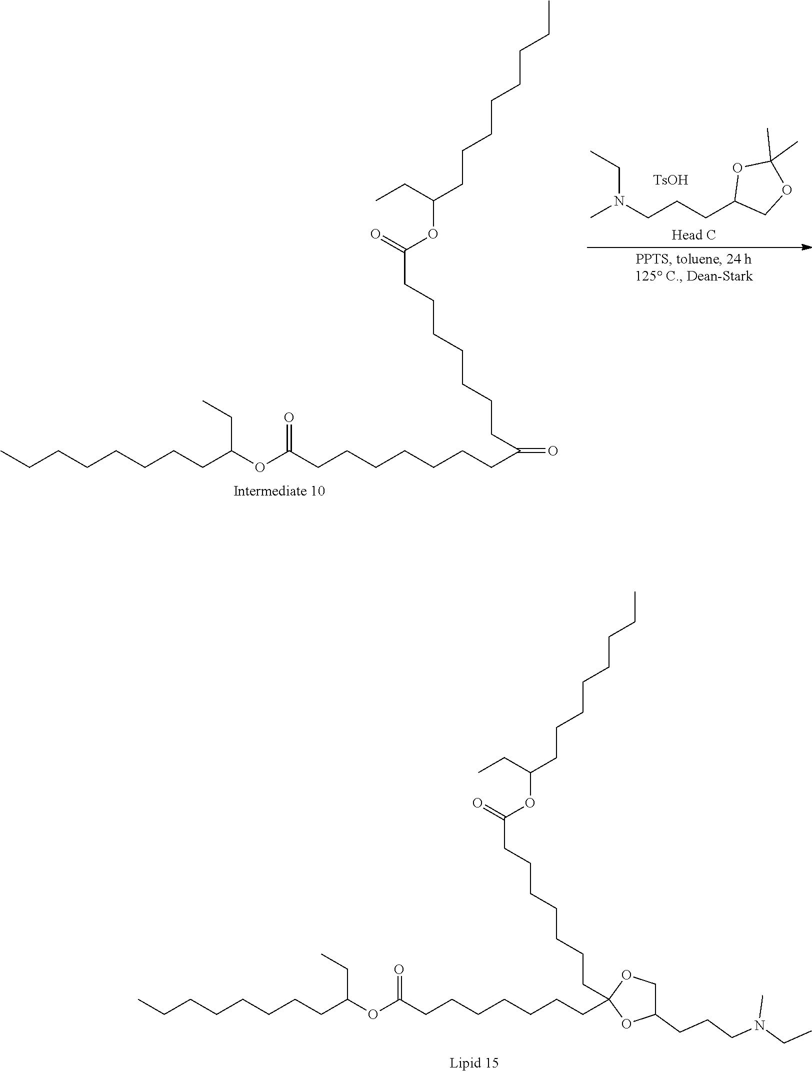

To a mixture of intermediate 10 (500 mg, 802.57 μmol, 1 eq.) and Head C (599.53 mg, 1.61 mmol, 2 eq., TsOH salt) in toluene (15 mL) was added PPTS (100.84 mg, 401.28 μmol, 0.5 eq.) and PTSA (45.80 mg, 240.77 μmol, 0.3 eq.), and then the mixture was stirred at 125° C. for 12 hours equipped with Dean-Stark. The reaction mixture was poured into sat. NaHCO₃ (aq., 40 mL) and extracted with EtO Ac (20 mL*2). The combined organic phases were washed with brine (20 mL), dried over anhydrous Na₂SO₄, filtered and concentrated under vacuum. The residue was purified by flash silica gel chromatography (SiO₂, PE/EA=10/1 to 5/1, eluent with 0.1% TEA), followed by another column purification (SiO₂, A/B=100/1 to 5/1, phase A: PE:DCM=3:1, phase B: EtOAc, eluent with 0.1% TEA) to give Lipid 15 (200.4 mg, 32.26% yield, 99% purity) as a yellow oil.

LCMS: m/z 766.7 [M+1]⁺. ¹HNMR: (400 MHz, CHLOROFORM-d) δ ppm 4.75-4.88 (m, 2H), 3.98-4.13 (m, 2H), 3.40-3.49 (m, 1H), 2.37-2.55 (m, 4H), 2.20-2.34 (m, 7H), 1.47-1.69 (m, 24H), 1.21-1.41 (m, 43H), 1.04-1.17 (m, 3H), 0.83-0.92 (m, 12H).

### Synthesis of Lipid 16

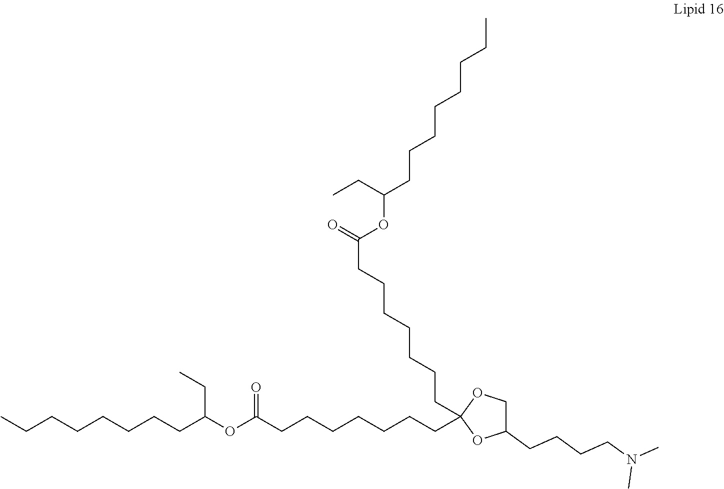 [SMILES: CCCCCCCCC(CC)OC(=O)CCCCCCCC1(CCCCCCCC(=O)OC(CC)CCCCCCCC)OCC(CCCCN(C)C)O1]

A scheme for the synthesis of Lipid 16 is provided in Scheme 15 below.

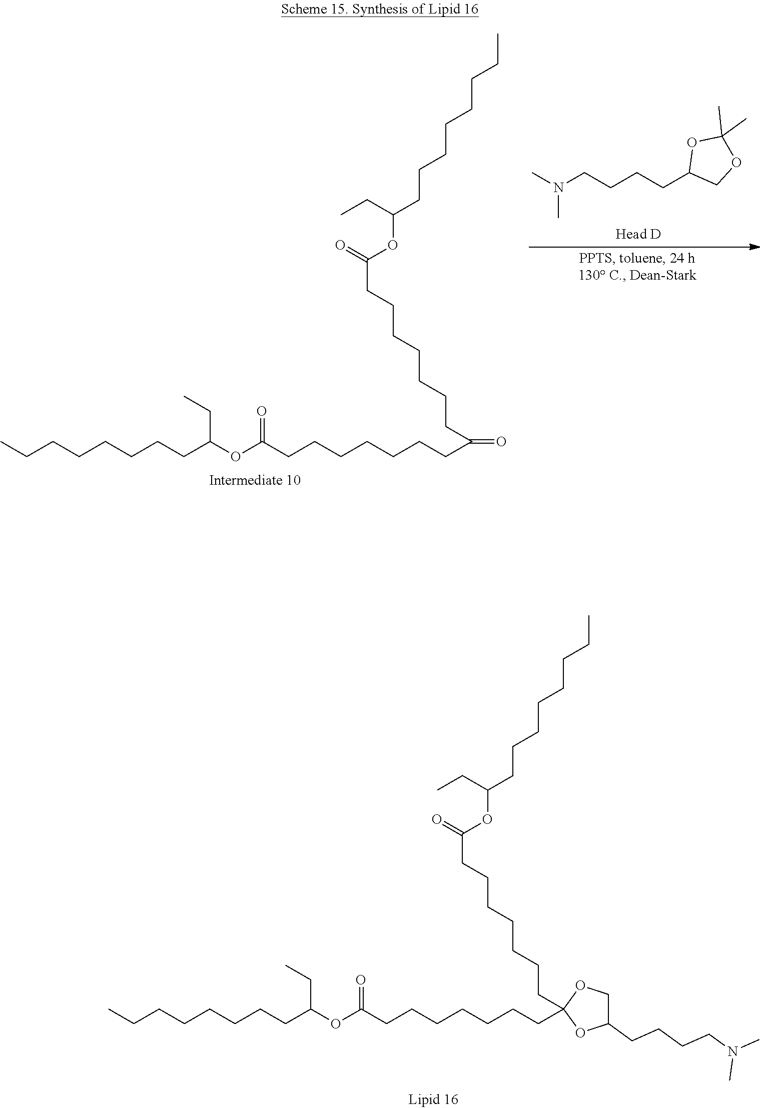

### Procedure for Preparation of Lipid 16

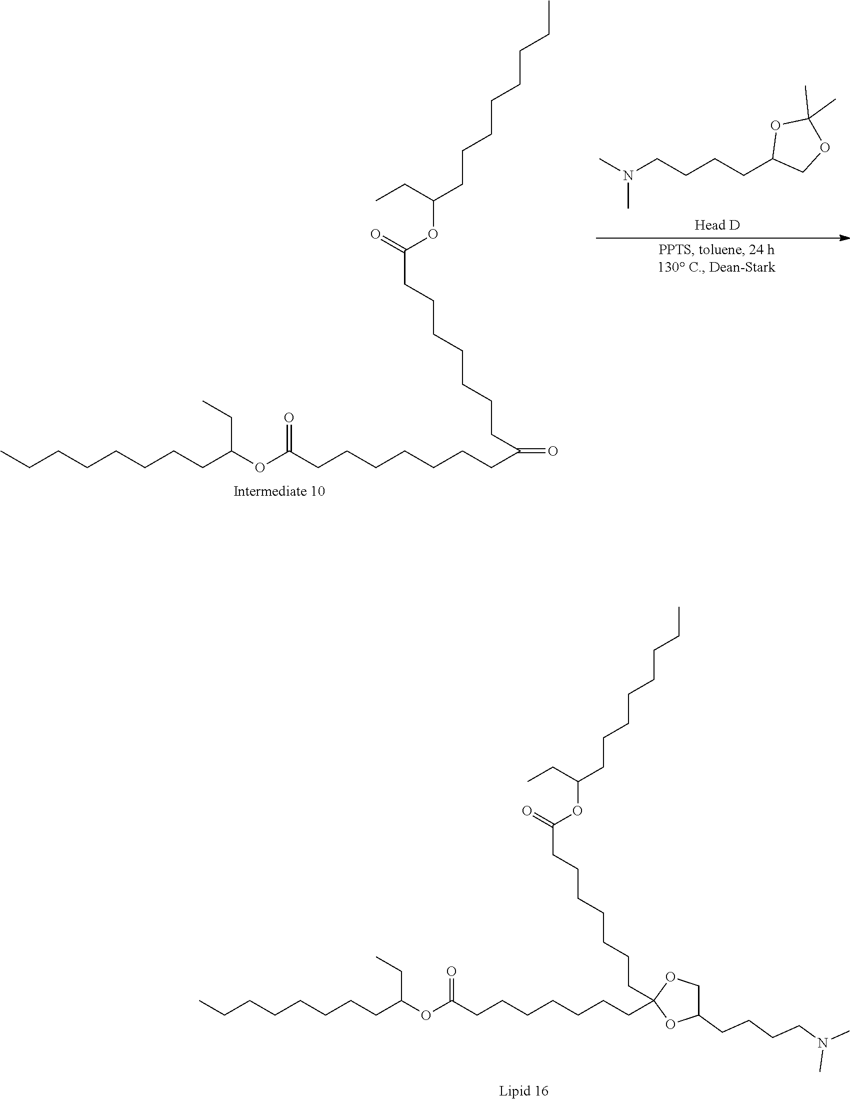

To a mixture of intermediate 10 (665.00 mg, 1.07 mmol, 1 eq.) and Head D (797.37 mg, 2.13 mmol, 2.00 eq., TsOH salt) in toluene (20 mL) was added PPTS (134.12 mg, 533.71 μmol, 0.5 eq.) and PTSA (81.22 mg, 426.97 μmol, 0.4 eq.). The mixture was stirred at 130° C. for 16 hours equipped with Dean-Stark. The reaction mixture was poured into sat. NaHCO₃ (aq., 10 mL) and extracted with EtOAc (20 mL*3). The combined organic layers were washed with brine (10 mL), dried over anhydrous Na₂SO₄, filtered and concentrated under reduced pressure. The residue was purified by column chromatography (SiO₂, A/B=100/1 to 6/1, phase A: PE:DCM=3:1, phase B: EtOAc, eluent with 0.1% TEA) to give Lipid 16 (216 mg, 26.41% yield) as a colorless oil.

Special LCMS: m/z 766.6 [M+1]⁺. ¹HNMR: (400 MHz, CHLOROFORM-d) δ ppm 4.84-4.77 (m, 2H), 4.07-3.99 (m, 2H), 3.46-3.39 (m, 1H), 2.30-2.23 (m, 6H), 2.21 (s, 6H), 1.65-1.45 (m, 20H), 1.38-1.21 (m, 42H), 0.90-0.84 (m, 12H).

### Synthesis of Lipid 17

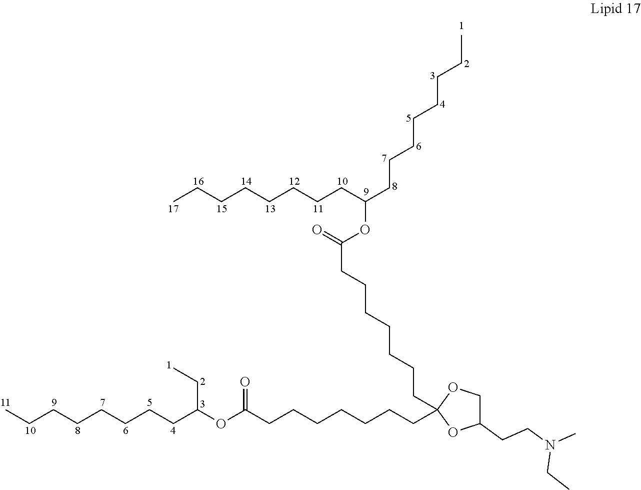 [SMILES: CCCCCCCCC(CC)OC(=O)CCCCCCCC1(CCCCCCCC(=O)OC(CCCCCCCC)CCCCCCCC)OCC(CCN(C)CC)O1]

A scheme for the synthesis of Lipid 17 is provided in Scheme 16 below.

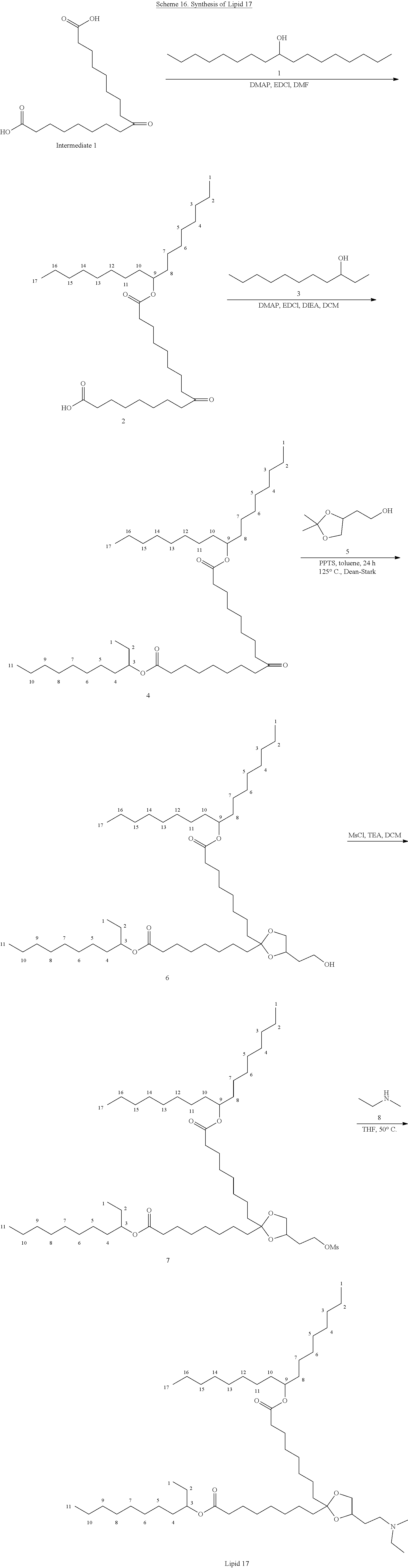

### Procedure for Preparation of Compound 2

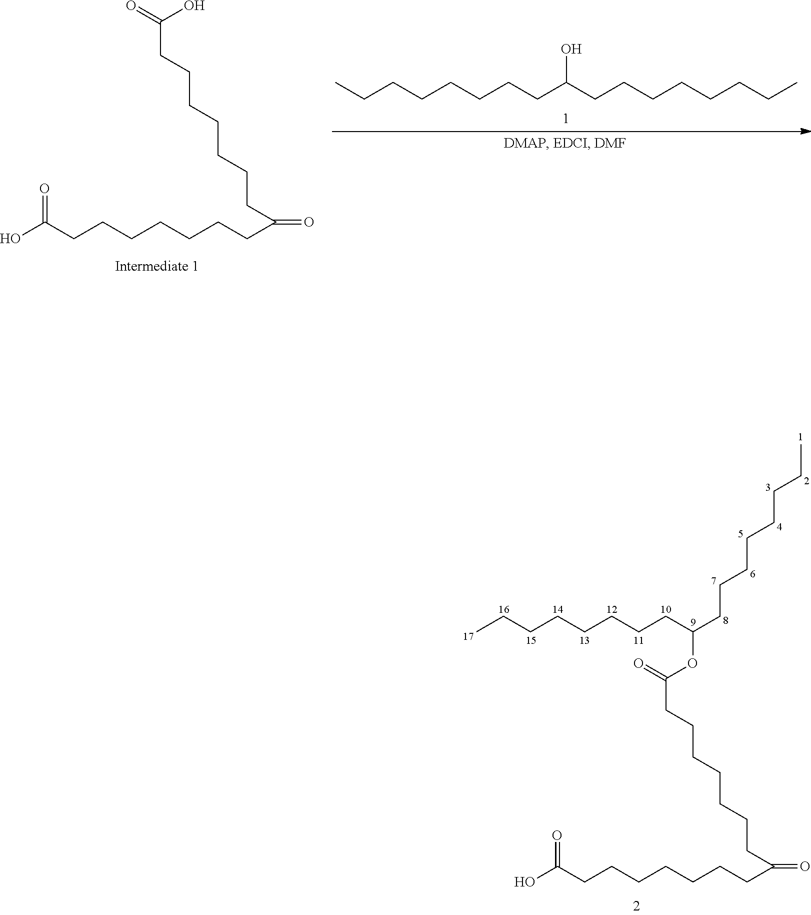 [SMILES: O=C(O)CCCCCCCC(=O)CCCCCCCC(=O)O ; CCCCCCCCC(O)CCCCCCCC ; CCCCCCCCC(CCCCCCCC)OC(=O)CCCCCCCC(=O)CCCCCCCC(=O)O]

To a mixture of intermediate 1 (9.19 g, 29.24 mmol, 1.5 eq.) and compound 1 (5 g, 19.50 mmol, 1 eq.) in DMF (40 mL) was added DMAP (2.62 g, 21.45 mmol, 1.1 eq.) and EDCI (4.86 g, 25.34 mmol, 1.3 eq.). The mixture was stirred at 25° C. for 12 hours. The mixture was diluted with H₂O (10 mL) and extracted with DCM (20 mL*2). The combined organic layers were washed with brine (20 mL), dried over anhydrous Na₂SO₄, filtered and concentrated under reduced pressure. The residue was purified by column chromatography (SiO₂, Petroleum ether/Ethyl acetate=I/O to 100/1) to give compound 2 (4 g, 37.11% yield) as a yellow oil.

¹HNMR: (400 MHz, CHLOROFORM-d) δ ppm 4.87 (quin, J=6.2 Hz, 1H), 2.43-2.25 (m, 8H), 1.66-1.47 (m, 12H), 1.34-1.22 (m, 36H), 0.88 (t, J=6.6 Hz, 6H).

### Procedure for Preparation of Compound 4

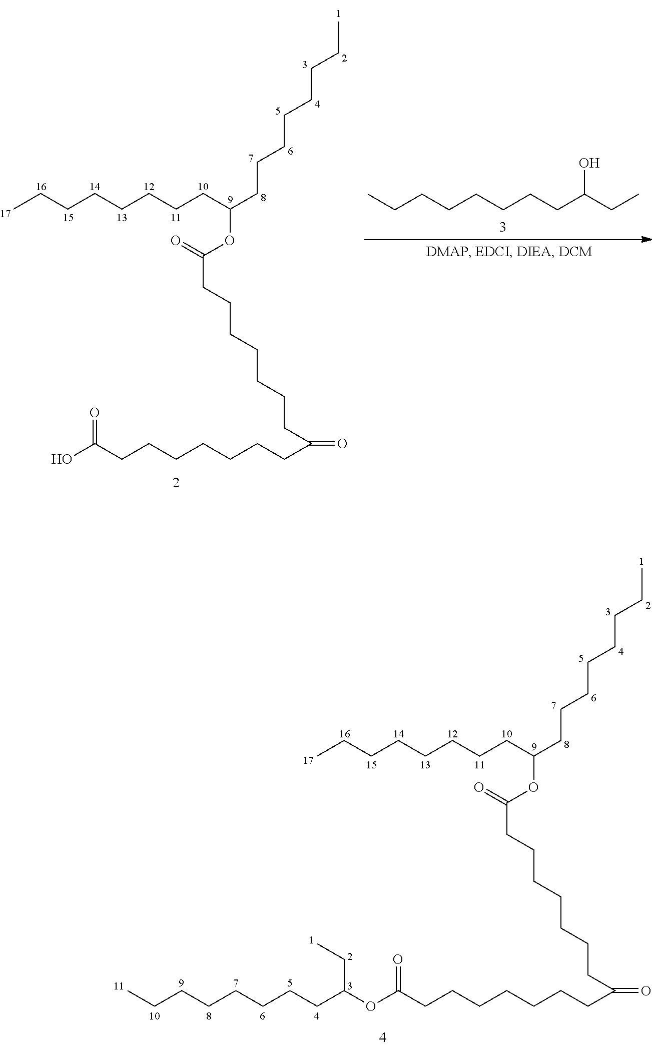 [SMILES: CCCCCCCCC(CCCCCCCC)OC(=O)CCCCCCCC(=O)CCCCCCCC(=O)O ; CCCCCCCCC(O)CC ; CCCCCCCCC(CC)OC(=O)CCCCCCCC(=O)CCCCCCCC(=O)OC(CCCCCCCC)CCCCCCCC]

To a mixture of compound 2 (1.5 g, 2.71 mmol, 1 eq.) and compound 3 (467.49 mg, 2.71 mmol, 1 eq.) in DCM (30 mL) was added DMAP (331.46 mg, 2.71 mmol, 1 eq.), EDCI (1.04 g, 5.43 mmol, 2 eq.) and DIEA (1.40 g, 10.85 mmol, 1.89 mL, 4 eq.). The mixture was stirred at 40° C. for 16 hours. The mixture was diluted with H₂O (20 mL) and extracted with DCM (30 mL*2). The combined organic layers were washed with brine (20 mL), dried over anhydrous Na₂SO₄, filtered and concentrated under reduced pressure. The residue was purified by column chromatography (SiO₂, Petroleum ether/Ethyl acetate=I/O to 100/1) to give compound 4 (1 g, 52.12% yield) as a colorless oil.

¹HNMR: (400 MHz, CHLOROFORM-d) δ ppm 4.90-4.78 (m, 2H), 2.37 (t, J=7.4 Hz, 4H), 2.28 (m, J=3.8, 7.6 Hz, 4H), 1.66-1.48 (m, 17H), 1.37-1.22 (m, 50H), 0.92-0.84 (m, 12H).

### Procedure for Preparation of Compound 6

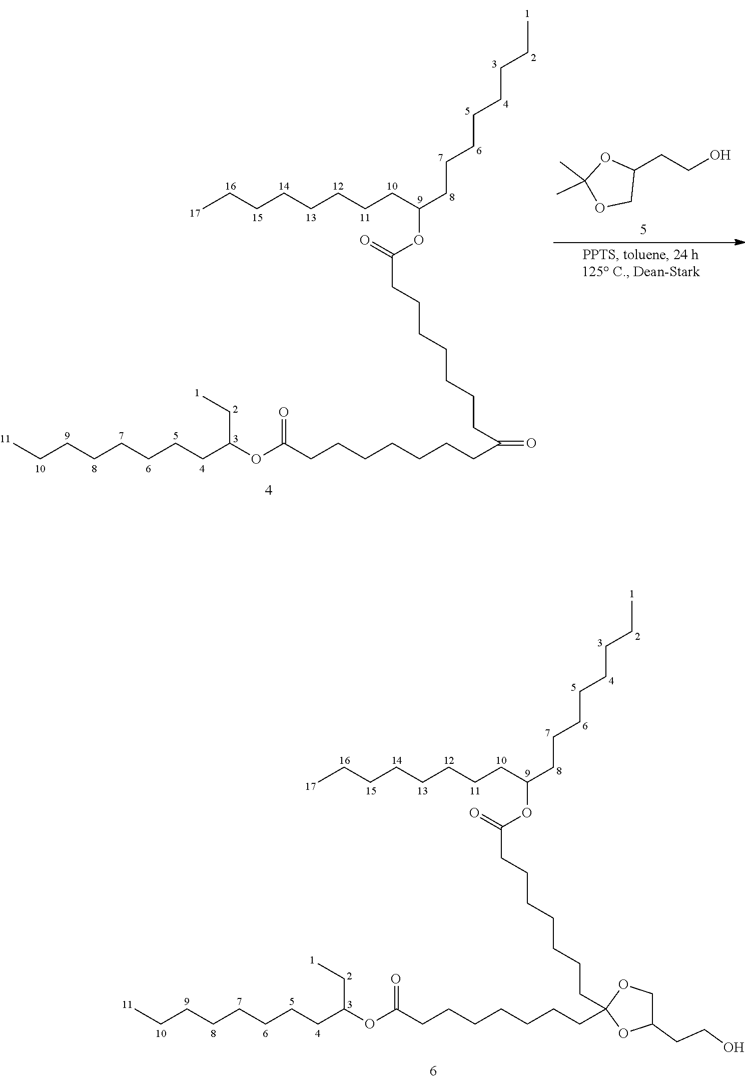

To a mixture of compound 4 (500 mg, 707.06 μmol, 1 eq.) and compound 5 (258.40 mg, 1.77 mmol, 250.63 μL, 2.5 eq.) in toluene (20 mL) was added PPTS (53.31 mg, 212.12 μmol, 0.3 eq.). The mixture was stirred at 130° C. for 12 hours equipped with Dean-Stark. The mixture was quenched by addition of sat. NaHCO₃ (aq., 10 mL) at 25° C., and then diluted with H₂O (5 mL) and extracted with DCM (20 mL*2). The combined organic layers were washed with Brine (10 mL*2), dried over anhydrous Na₂SO₄, filtered and concentrated under reduced pressure. The residue was purified by column chromatography (SiO₂, Petroleum ether/Ethyl acetate=10/1 to 3/1) to give compound 6 (410 mg, 72.92% yield) as a colourless oil.

¹HNMR: (400 MHz, CHLOROFORM-d) δ ppm 4.90-4.78 (m, 2H), 4.27-4.20 (m, 1H), 4.16-4.06 (m, 1H), 3.85-3.76 (m, 2H), 3.53 (t, J=7.8 Hz, 1H), 2.28 (dt, J=4.0, 7.6 Hz, 4H), 1.89-1.75 (m, 2H), 1.65-1.48 (m, 16H), 1.38-1.22 (m, 54H), 0.92-0.85 (m, 12H).

### Procedure for Preparation of Compound 7

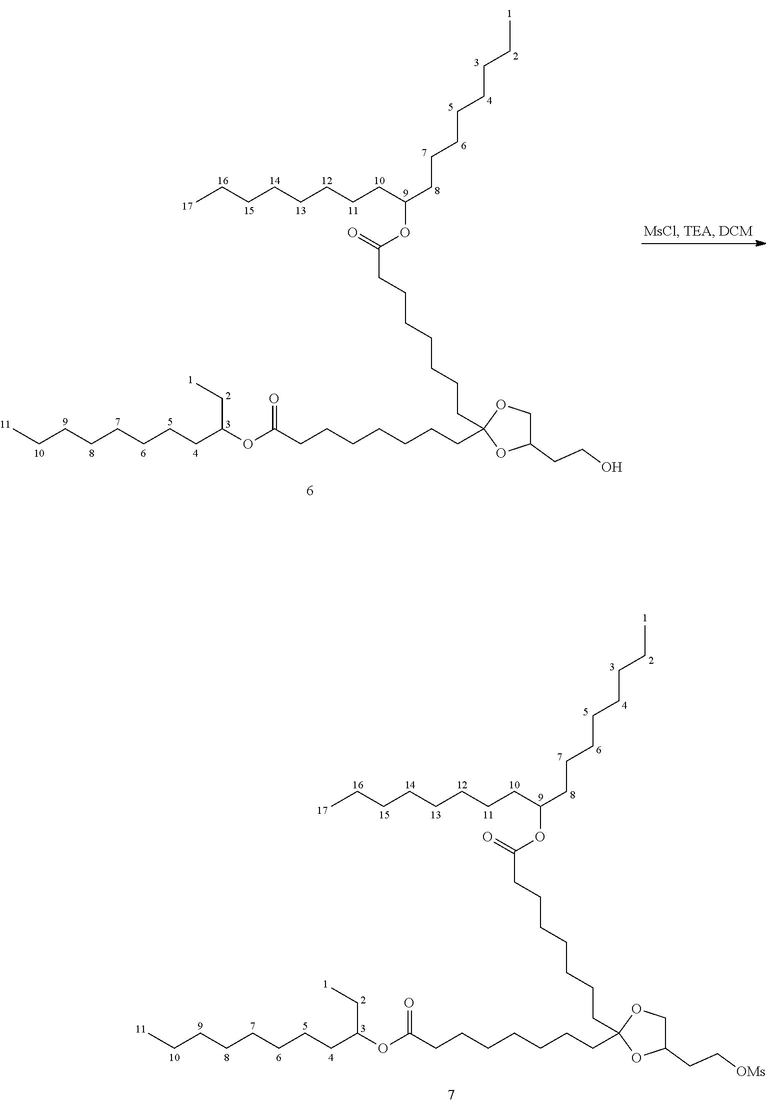 [SMILES: CCCCCCCCC(CC)OC(=O)CCCCCCCC1(CCCCCCCC(=O)OC(CCCCCCCC)CCCCCCCC)OCC(CCO)O1 ; CCCCCCCCC(CC)OC(=O)CCCCCCCC1(CCCCCCCC(=O)OC(CCCCCCCC)CCCCCCCC)OCC(CCOS(C)(=O)=O)O1]

To a solution of compound 6 (410 mg, 515.55 μmol, 1 eq.) in DCM (5 mL) was added TEA (260.84 mg, 2.58 mmol, 358.79 μL, 5 eq.) and MsCl (236.23 mg, 2.06 mmol, 159.61 μL, 4 eq.) at 0° C. The mixture was stirred at 25° C. for 2 hours. The mixture was poured into ice-water (10 mL) and extracted with DCM (10 mL*3). The combined organic layers were washed with brine (10 mL) and concentrated under reduced pressure to give compound 7 (540 mg, crude) as a yellow oil.

¹HNMR: EW48174-292-PIA (400 MHz, CHLOROFORM-d) δ ppm 4.90-4.78 (m, 2H), 4.44-4.30 (m, 2H), 4.23-4.07 (m, 2H), 3.58-3.40 (m, 2H), 3.21-3.11 (m, 2H), 3.03 (s, 3H), 2.29 (dt, J=4.0, 7.6 Hz, 4H), 1.63-1.48 (m, 15H), 1.35-1.23 (m, 53H), 1.05 (br d, J=6.4 Hz, 1H), 0.92-0.85 (m, 12H).

### Procedure for Preparation of Lipid 17

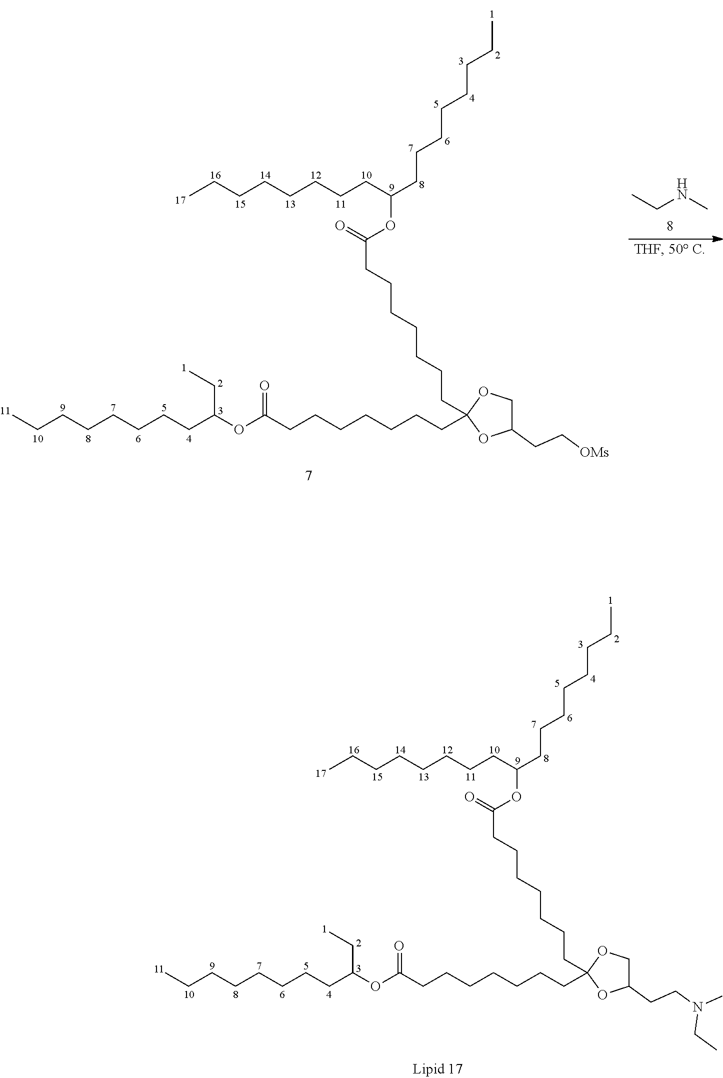

To a solution of compound 7 (535 mg, 612.58 μmol, 1 eq.) in THF (8 mL) was added compound 8 (1.38 g, 23.28 mmol, 2 mL, 38.00 eq.). The mixture was stirred at 50° C. for 16 hours. The mixture was poured into sat. NaHCO₃ (aq., 10 mL) and extracted with EtOAc (20 mL*3). The combined organic layers were washed with brine (10 mL), dried over anhydrous Na₂SO₄, filtered and concentrated under reduced pressure. The residue was purified by column chromatography (SiO₂, A/B=100/1 to 5/1, phase A: PE:DCM=3:1, phase B: EtOAc, eluent with 0.1% TEA) twice to give Lipid 17 (340 mg, 66.36% yield, 97% purity) as a colorless oil.

LCMS: m/z: 837.0 [M+1]⁺. ¹HNMR: (400 MHz, CHLOROFORM-d) δ ppm 4.90-4.79 (m, 2H), 4.12-4.03 (m, 211), 3.73 (q, J=7.0 Hz, 2H), 3.51-3.45 (m, 1H), 2.52-2.33 (m, 4H), 2.32-2.25 (m, 4H), 2.22 (s, 3H), 1.86-1.77 (m, 1H), 1.67-1.44 (m, 19H), 1.31-1.23 (m, 48H), 1.06 (t, J=7.2 Hz, 3H), 0.91-0.85 (m, 12H).

### Synthesis of Lipid 18

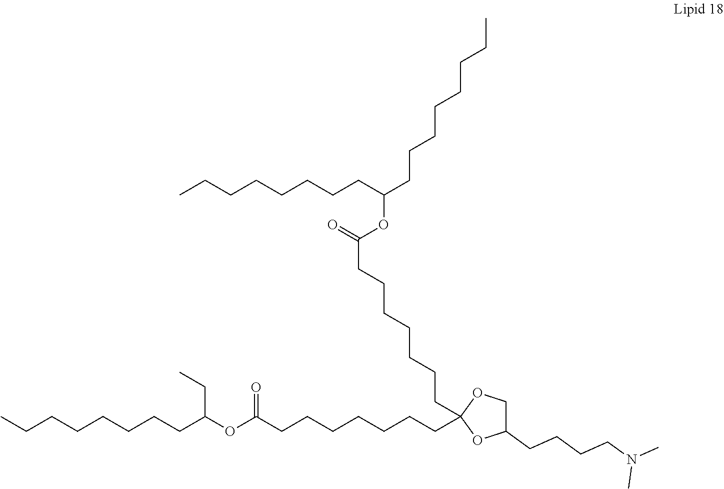 [SMILES: CCCCCCCCC(CC)OC(=O)CCCCCCCC1(CCCCCCCC(=O)OC(CCCCCCCC)CCCCCCCC)OCC(CCCCN(C)C)O1]

A scheme for the synthesis of Lipid 18 is shown in Scheme 17 below.

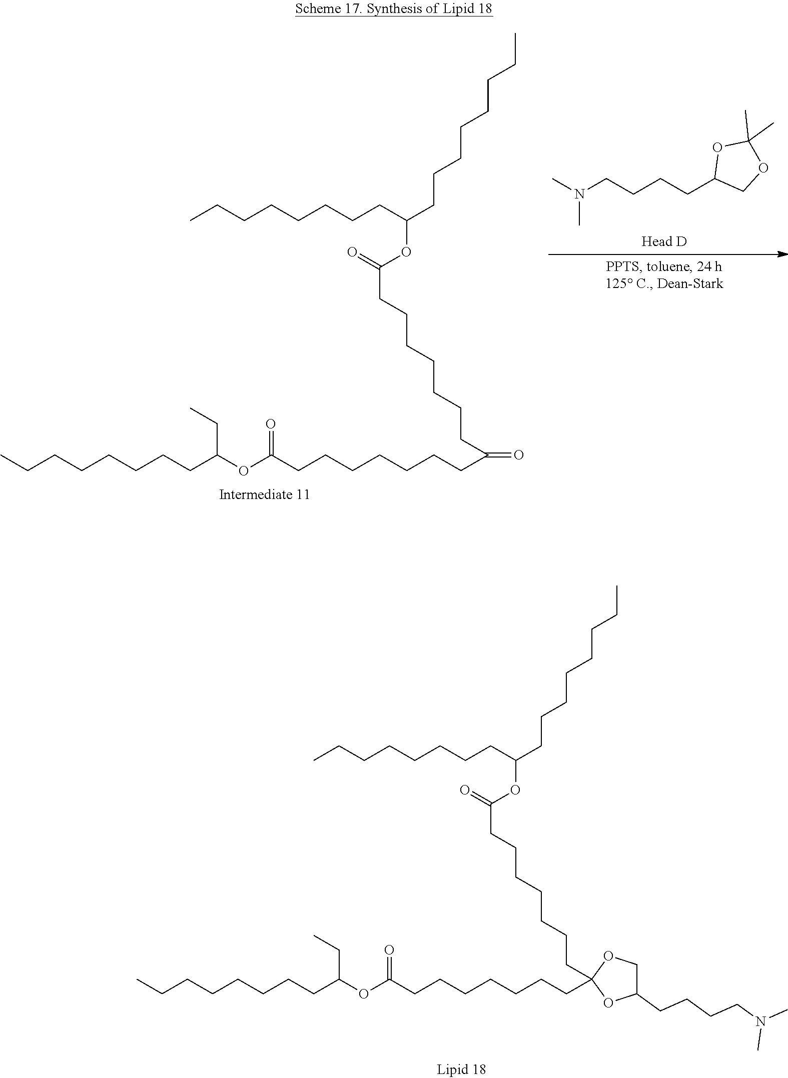

### Procedure for Preparation of Lipid 18

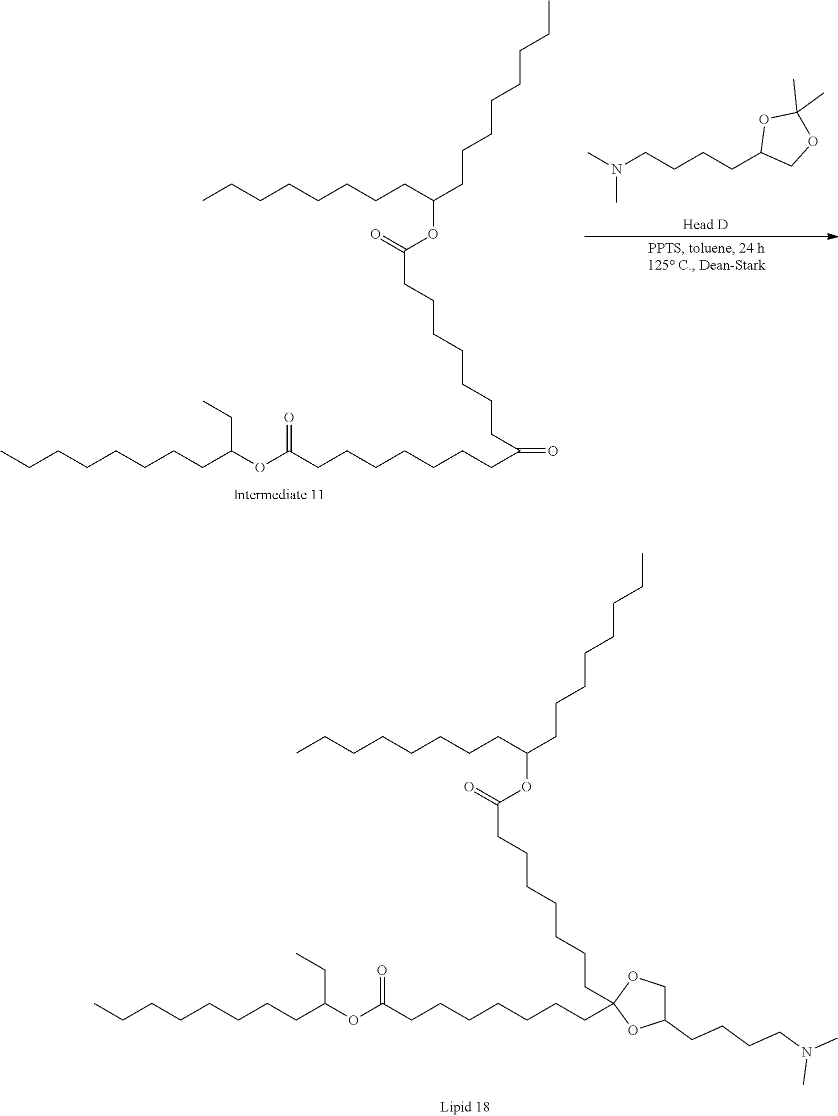

To a mixture of intermediate 11 (920 mg, 1.30 mmol, 1 eq.) and Head D (1.4 g, 3.75 mmol, 2.88 eq., TsOH salt) in toluene (20 mL) was added PPTS (163.47 mg, 650.49 μmol, 0.5 eq.) and PTSA (98.99 mg, 520.39 μmol, 0.4 eq.). The mixture was stirred at 130° C. for 16 hours equipped with Dean-Stark. The mixture was diluted with H₂O (10 mL) and extracted with DCM (20 mL*2). The combined organic layers were washed with brine (20 mL), dried over anhydrous Na₂SO₄, filtered and concentrated under reduced pressure. The residue was purified by column chromatography (SiO₂, PE/EA=10/1 to 5/1, eluent with 0.1% TEA), followed by another column purification (SiO₂, A/B=100/1 to 5/1, phase A: PE:DCM=3:1, phase B: EtOAc, eluent with 0.1% TEA) to give Lipid 18 (435 mg, 39.32% yield) as a colourless oil.

Special LCMS: m/z: 851.0 [M+1]⁺. ¹HNMR: (400 MHz, CHLOROFORM-d) δ ppm 4.90-4.78 (m, 2H), 4.07-3.99 (m, 2H), 3.46-3.40 (m, 1H), 2.30-2.24 (m, 6H), 2.22 (s, 6H), 1.65-1.44 (m, 21H), 1.34-1.20 (m, 53H), 0.92-0.84 (m, 12H).

### Synthesis of Lipid 19

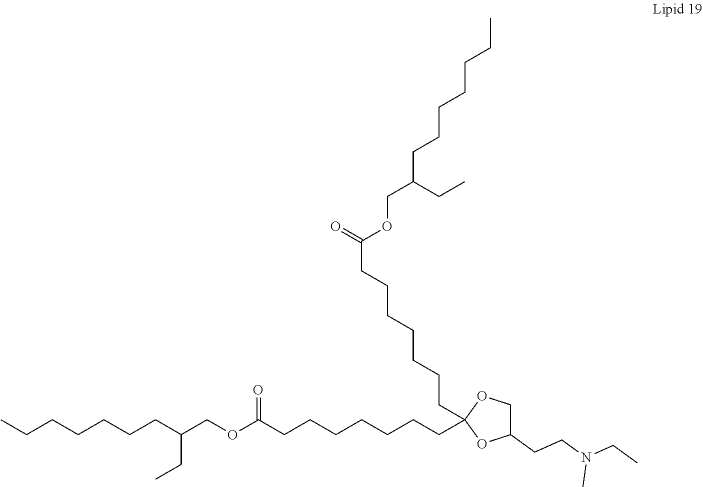 [SMILES: CCCCCCCC(CC)COC(=O)CCCCCCCC1(CCCCCCCC(=O)OCC(CC)CCCCCCC)OCC(CCN(C)CC)O1]

A scheme for the synthesis of Lipid 19 is provided below in Scheme 18.

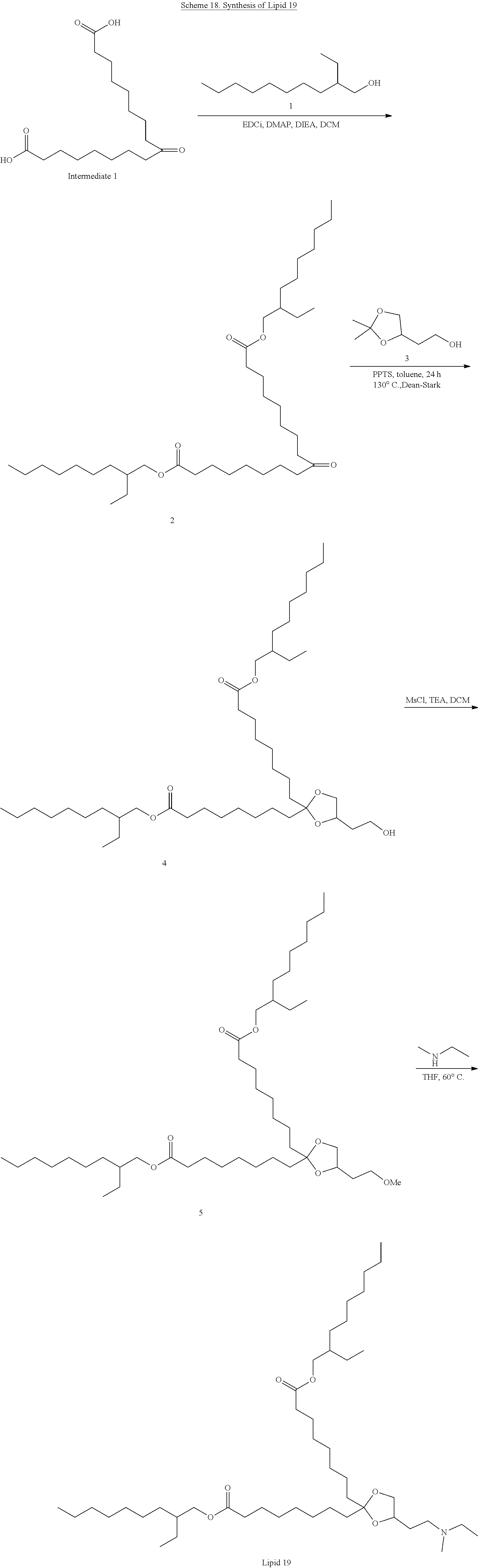

### Procedure for Preparation of Compound 2

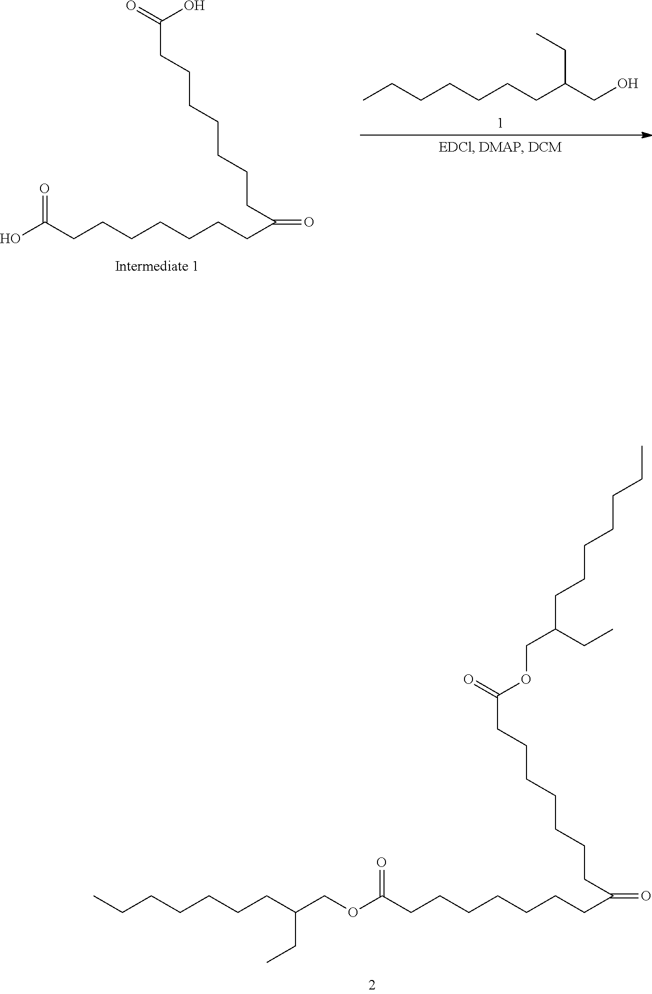 [SMILES: O=C(O)CCCCCCCC(=O)CCCCCCCC(=O)O ; CCCCCCCC(CC)CO ; CCCCCCCC(CC)COC(=O)CCCCCCCC(=O)CCCCCCCC(=O)OCC(CC)CCCCCCC]

To a mixture of intermediate 1 (780 mg, 2.48 mmol, 1 eq.) and compound 1 (897.66 mg, 5.21 mmol, 2.1 eq.) in DCM (10 mL) was added EDCI (2.38 g, 12.40 mmol, 5 eq.), DIEA (1.60 g, 12.40 mmol, 2.16 mL, 5 eq.) and DMAP (151.54 mg, 1.24 mmol, 0.5 eq.). The mixture was stirred at 40° C. for 12 hours. The mixture was diluted with H₂O (20 mL) and extracted with DCM (30 mL*2). The combined organic layers were washed with brine (20 mL), dried over anhydrous Na₂SO₄, filtered and concentrated under reduced pressure. The residue was purified by column chromatography (SiO₂, Petroleum ether/Ethyl acetate=I/O to 100/1) to give compound 2 (860 mg, 55.64% yield) as a yellow oil.

¹HNMR: EW48174-270-PIA (400 MHz, CHLOROFORM-d) δ ppm 4.02-3.94 (m, 4H), 2.40-2.26 (m, 8H), 1.74-1.49 (m, 11H), 1.39-1.21 (m, 41H), 0.92-0.85 (m, 12H).

### Procedure for Preparation of Compound 4

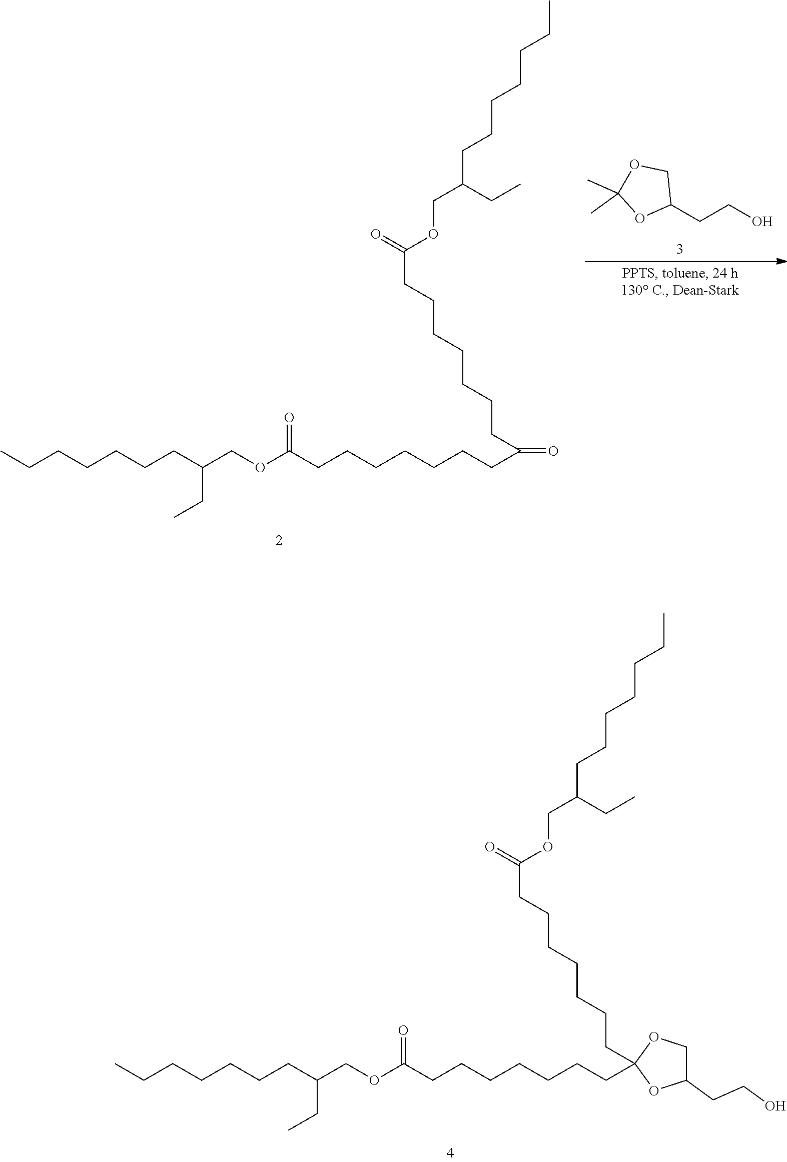

To a mixture of compound 2 (400 mg, 642.06 μmol, 1 eq.) and compound 3 (234.65 mg, 1.61 mmol, 227.59 μL, 2.5 eq.) in toluene (15 mL) was added PPTS (48.40 mg, 192.62 μmol, 0.3 eq.). The mixture was stirred at 130° C. for 12 hours equipped with Dean-Stark. The mixture was diluted with H₂O (20 mL) and extracted with DCM (30 mL*2). The combined organic layers were washed with brine (20 mL), dried over anhydrous Na₂SO₄, filtered and concentrated under reduced pressure. The residue was purified by column chromatography (SiO₂, Petroleum ether/Ethyl acetate=10/1 to 5/1) to give compound 4 (235 mg, 51.47% yield) as a yellow oil.

¹HNMR: (400 MHz, CHLOROFORM-d) δ ppm 4.26-4.19 (m, 1H), 4.10-3.93 (m, 5H), 3.84-3.75 (m, 2H), 3.52 (t, J=8.0 Hz, 1H), 2.29 (t, J=7.6 Hz, 5H), 1.87-1.76 (m, 2H), 1.65-1.52 (m, 10H), 1.40-1.24 (m, 45H), 0.94-0.85 (m, 12H).

### Procedure for Preparation of Compound 5

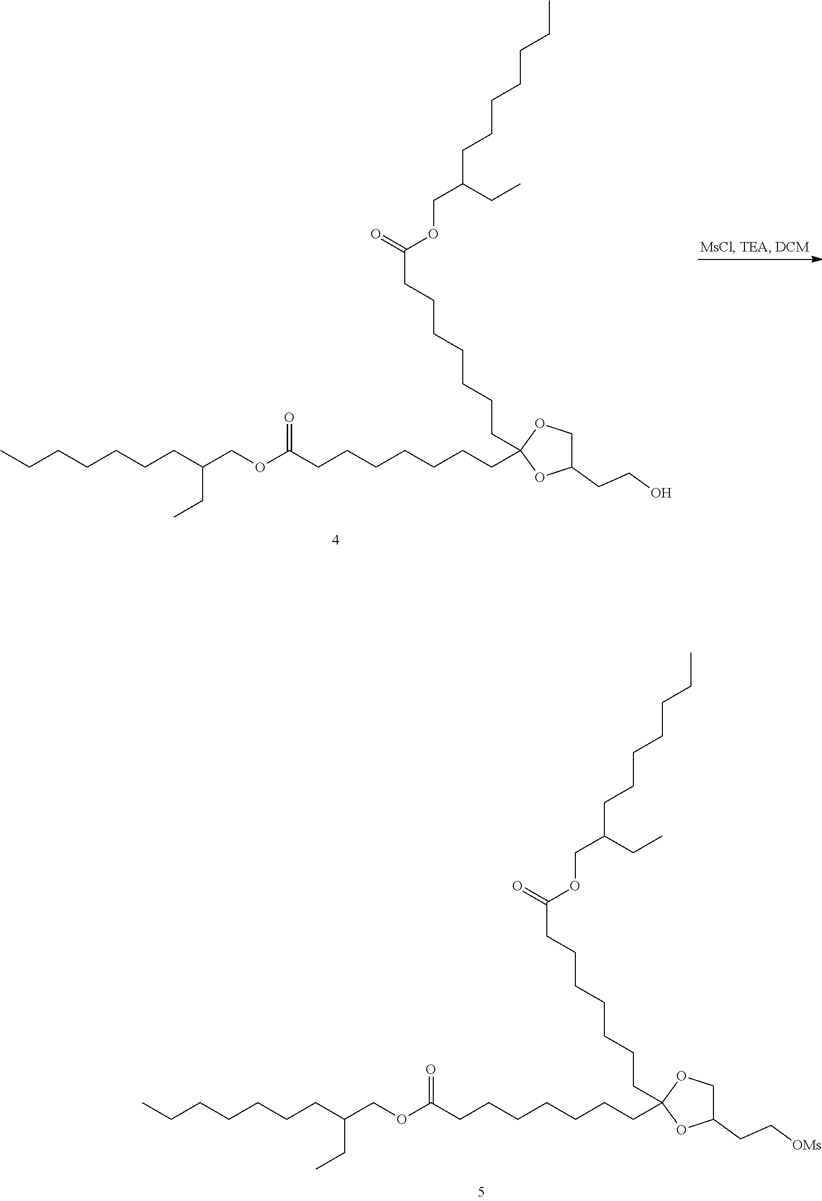 [SMILES: CCCCCCCC(CC)COC(=O)CCCCCCCC1(CCCCCCCC(=O)OCC(CC)CCCCCCC)OCC(CCO)O1 ; CCCCCCCC(CC)COC(=O)CCCCCCCC1(CCCCCCCC(=O)OCC(CC)CCCCCCC)OCC(CCOS(C)(=O)=O)O1]

To a solution of compound 4 (235 mg, 330.47 μmol, 1 eq.) in DCM (5 mL) was added TEA (167.20 mg, 1.65 mmol, 229.99 μL, 5 eq.) and MsCl (189.28 mg, 1.65 mmol, 127.89 μL, 5 eq.) at 0° C. The mixture was stirred at 25° C. for 2 hours. The mixture was poured into ice-water (10 mL), and extracted with DCM (10 mL*3). The combined organic layers were washed with brine (10 mL), dried over anhydrous Na₂SO₄, filtered and concentrated under reduced pressure to give compound 5 (446 mg, crude) as a yellow oil, which was used directly for next step without further purification.

¹HNMR: (400 MHz, CHLOROFORM-d) δ ppm 4.43-4.29 (m, 2H), 4.00-3.96 (m, 3H), 3.56-3.39 (m, 2H), 3.06-3.00 (m, 3H), 2.30 (t, J=7.6 Hz, 4H), 2.13-1.85 (m, 2H), 1.65-1.53 (m, 10H), 1.43-1.22 (m, 46H), 0.93-0.85 (m, 12H).

### Procedure for Preparation of Lipid 19

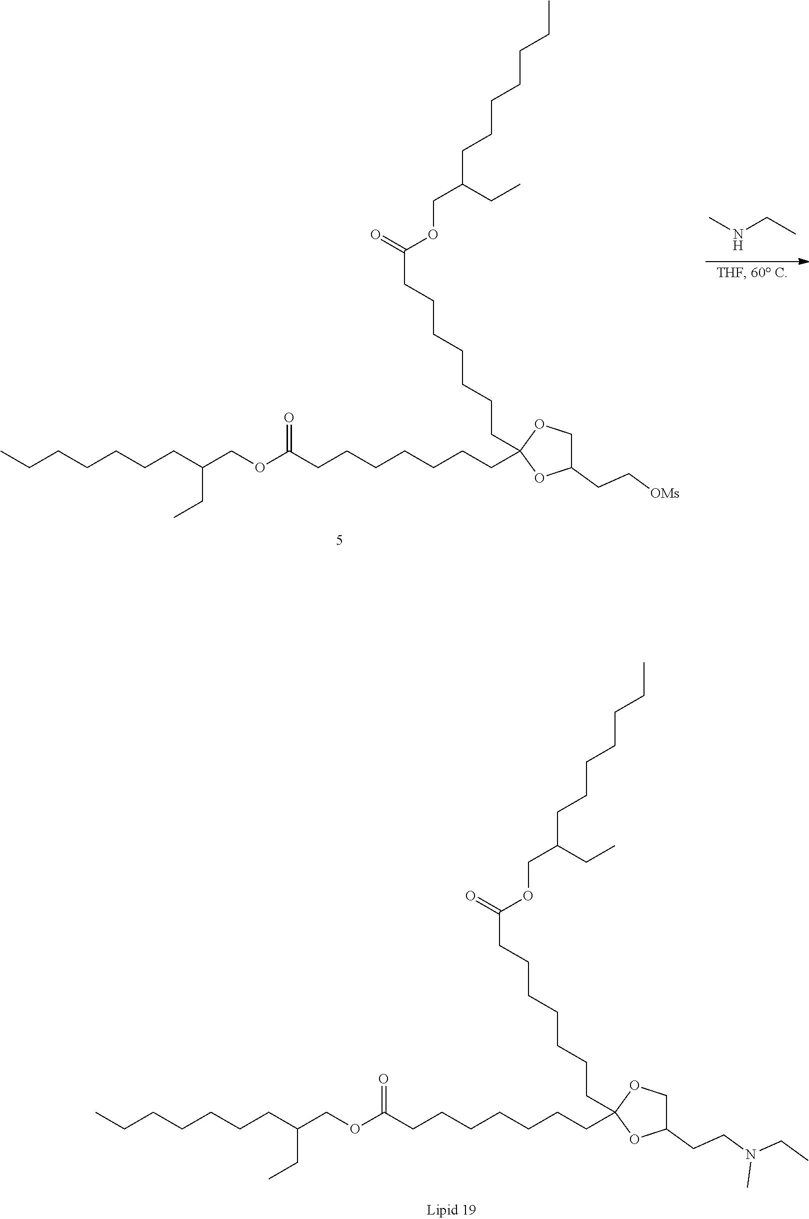

To a solution of compound 5 (440 mg, 557.53 μmol, 1 eq.) in THF (8 mL) was added N-methylethanamine (1.35 g, 22.76 mmol, 1.96 mL, 40.83 eq.). The mixture was stirred at 50° C. for 12 hours. The mixture was diluted with H₂O (20 mL) and extracted with DCM (30 mL*2). The combined organic layers were washed with brine (20 mL), dried over anhydrous Na₂SO₄, filtered and concentrated under reduced pressure. The residue was purified by column chromatography (SiO₂, PE/EA=10/1 to 5/1 eluant with 0.1% TEA), followed by another column purification (SiO₂, A/B=100/1 to 5/1, phase A: PE:DCM=3:1, phase B: EtOAc, eluent with 0.1% TEA), and then run the prep-NPLC {column: Welch Ultimate XB—SiOH 250*50*10 μm; mobile phase: [Hexane-EtOH (0.1% NH₃·H₂O)]; B %; 1%, isocratic elution mode} to give Lipid 19 (85.8 mg, 42.90% yield) as a colorless oil.

LCMS: m/z 752.9 [M+1]⁺. ¹HNMR: (400 MHz, CHLOROFORM-d) δ ppm 4.12-3.94 (m, 6H), 3.51-3.45 (m, 1H), 2.52-2.34 (m, 4H), 2.29 (t, J=7.6 Hz, 4H), 2.21 (s, 3H), 1.87-1.77 (m, 1H), 1.74-1.51 (m, 13H), 1.41-1.22 (m, 7H), 1.05 (t, J=7.2 Hz, 3H), 0.92-0.86 (m, 12H) Synthesis of Lipid 20

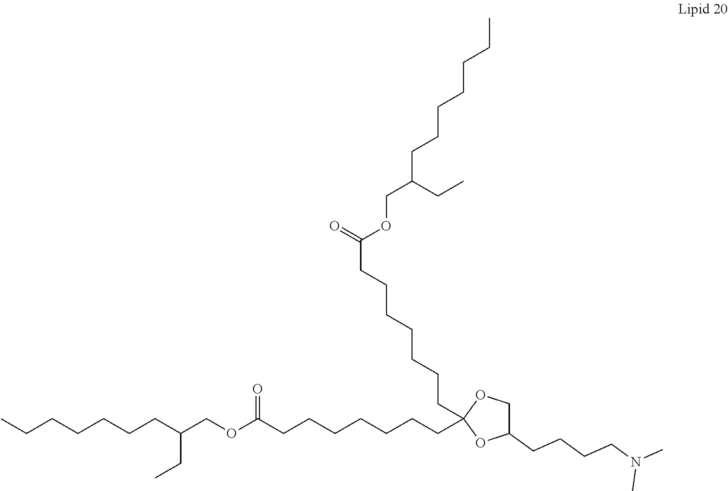 [SMILES: CCCCCCCC(CC)COC(=O)CCCCCCCC1(CCCCCCCC(=O)OCC(CC)CCCCCCC)OCC(CCCCN(C)C)O1]

A scheme for the synthesis of Lipid 20 is provided in Scheme 19 below.

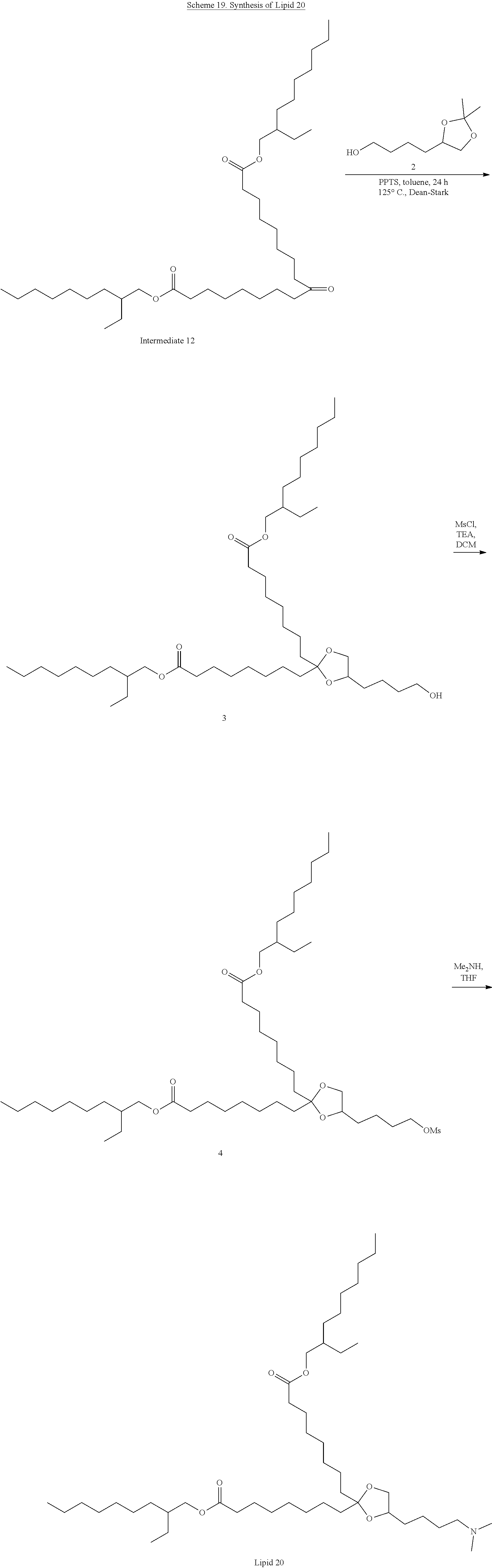 [SMILES: CCCCCCCC(CC)COC(=O)CCCCCCCC(=O)CCCCCCCC(=O)OCC(CC)CCCCCCC ; CC1(C)OCC(CCCCO)O1 ; CCCCCCCC(CC)COC(=O)CCCCCCCC1(CCCCCCCC(=O)OCC(CC)CCCCCCC)OCC(CCCCO)O1 ; CCCCCCCC(CC)COC(=O)CCCCCCCC1(CCCCCCCC(=O)OCC(CC)CCCCCCC)OCC(CCCCOS(C)(=O)=O)O1 ; CCCCCCCC(CC)COC(=O)CCCCCCCC1(CCCCCCCC(=O)OCC(CC)CCCCCCC)OCC(CCCCN(C)C)O1]

### Procedure for Preparation of Compound 3

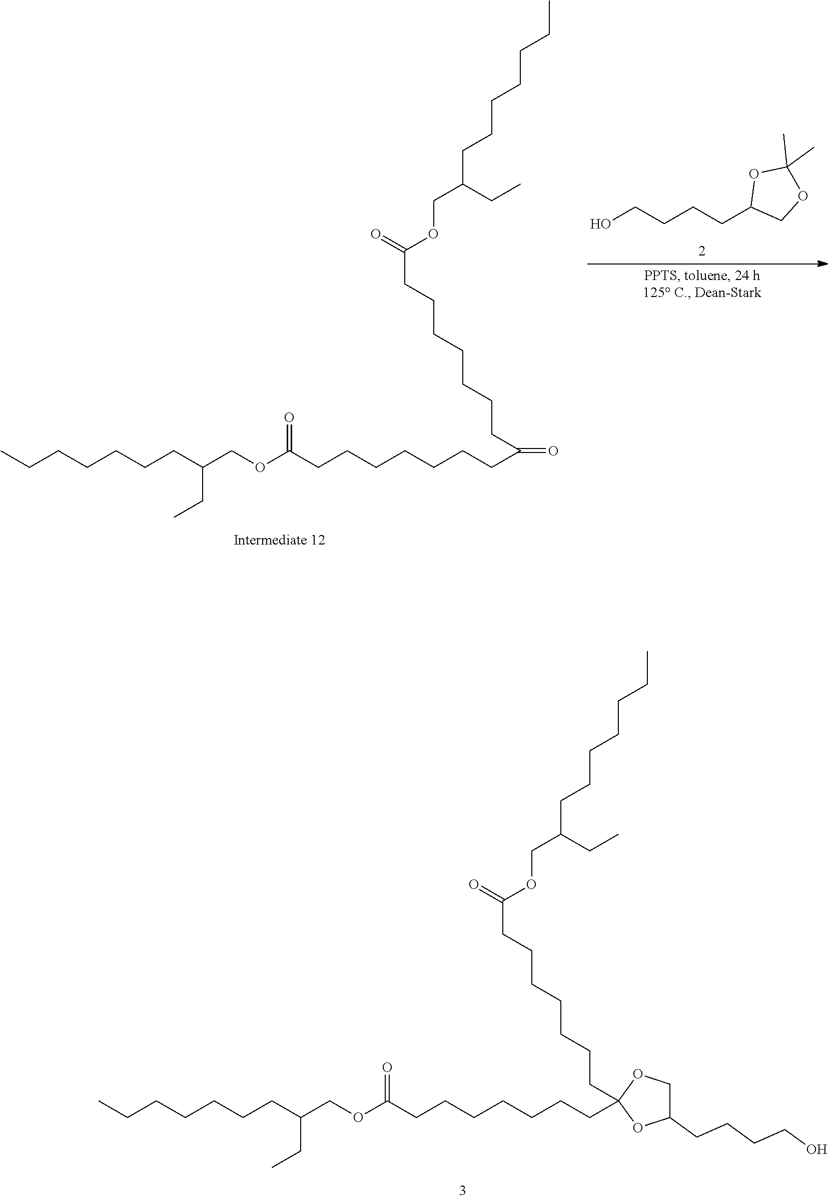 [SMILES: CCCCCCCC(CC)COC(=O)CCCCCCCC(=O)CCCCCCCC(=O)OCC(CC)CCCCCCC ; CC1(C)OCC(CCCCO)O1 ; CCCCCCCC(CC)COC(=O)CCCCCCCC1(CCCCCCCC(=O)OCC(CC)CCCCCCC)OCC(CCCCO)O1]

To a mixture of intermediate 12 (425 mg, 682.18 μmol, 1 eq.) and compound 2 (297.15 mg, 1.71 mmol, 2.5 eq.) in toluene (15 mL) was added PPTS (51.43 mg, 204.66 μmol, 0.3 eq.). The mixture was stirred at 130° C. for 12 hours equipped with Dean-Stark. The mixture was diluted with H₂O (20 mL) and extracted with DCM (30 mL*2). The combined organic layers were washed with brine (20 mL), dried over anhydrous Na₂SO₄, filtered and concentrated under reduced pressure to give compound 3 (310 mg, 61.48% yield) as a yellow oil.

¹HNMR: (400 MHz, CHLOROFORM-d) δ ppm 4.10-3.93 (m, 6H), 3.66 (t, J=6.4 Hz, 2H), 3.51-3.40 (m, 1H), 2.29 (t, J=7.5 Hz, 4H), 1.71-1.48 (m, 16H), 1.21 (br s, 45H), 0.88 (dt, J=2.0, 7.2 Hz, 12H).

### Procedure for Preparation of Compound 4

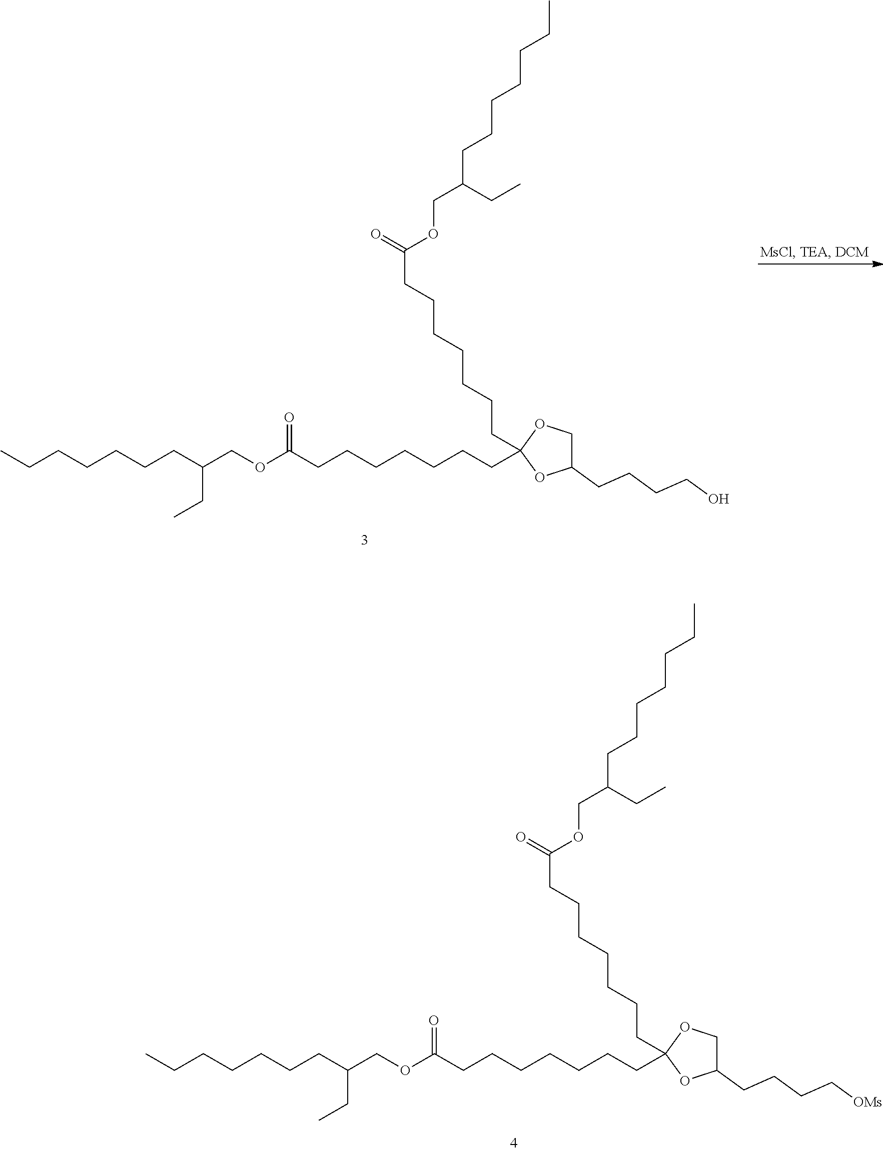 [SMILES: CCCCCCCC(CC)COC(=O)CCCCCCCC1(CCCCCCCC(=O)OCC(CC)CCCCCCC)OCC(CCCCO)O1 ; CCCCCCCC(CC)COC(=O)CCCCCCCC1(CCCCCCCC(=O)OCC(CC)CCCCCCC)OCC(CCCCOS(C)(=O)=O)O1]

To a solution of compound 3 (305 mg, 412.63 μmol, 1 eq.) in DCM (5 mL) was added TEA (208.77 mg, 2.06 mmol, 287.17 μL, 5 eq.) and MsCl (236.34 mg, 2.06 mmol, 159.69 μL, 5 eq.) at 0° C. The mixture was stirred at 25° C. for 2 hours. The mixture was poured into ice-water (10 mL), and extracted with DCM (10 mL*3). The combined organic layers were washed with brine (10 mL), dried over anhydrous Na₂SO₄, filtered and concentrated under reduced pressure to give compound 4 (576 mg, crude) as a yellow oil, which was used directly for next step without further purification.

¹HNMR: (400 MHz, CHLOROFORM-d) δ ppm 4.34-4.20 (m, 2H), 4.16-3.92 (m, 6H), 3.56-3.39 (m, 2H), 3.14 (s, 3H), 3.01 (s, 3H), 2.29 (t, J=7.6 Hz, 4H), 1.80 (quin, J=6.8 Hz, 2H), 1.71-1.57 (m, 8H), 1.54-1.53 (m, 1H), 1.52-1.24 (m, 47H), 0.95-0.82 (m, 12H).

### Procedure for Preparation of Lipid 20

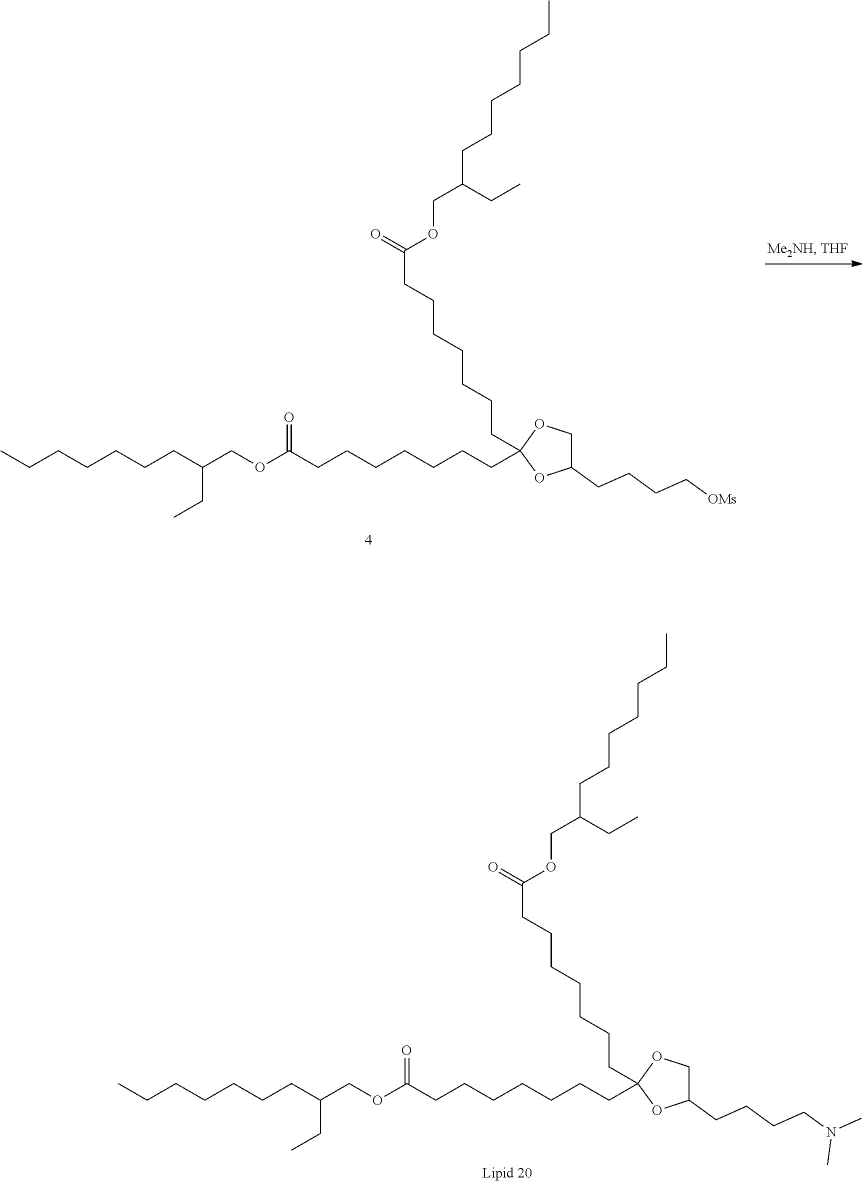 [SMILES: CCCCCCCC(CC)COC(=O)CCCCCCCC1(CCCCCCCC(=O)OCC(CC)CCCCCCC)OCC(CCCCOS(C)(=O)=O)O1 ; CCCCCCCC(CC)COC(=O)CCCCCCCC1(CCCCCCCC(=O)OCC(CC)CCCCCCC)OCC(CCCCN(C)C)O1]

A mixture of compound 4 (575 mg, 703.58 μmol, 1 eq.) in Me₂NH (2 M THF solution, 10 mL) was stirred at 50° C. for 16 hours. The reaction mixture was concentrated under reduced pressure. The residue was purified by column chromatography (SiO₂, PE/EA=10/1 to 5/1, eluent with 0.1% TEA), followed by another column purification (SiO₂, A/B=100/1 to 5/1, phase A; PE:DCM=3:1, phase B: EtOAc, eluent with 0.1% TEA), and then further purified by prep-NPLC {column: Welch Ultimate XB—SiOH 250*50*10 μm; mobile phase: [Hexane-EtOH (0.1% NH₃·H₂O]; B %: 1%, isocratic elution mode} to give Lipid 20 (84.2 mg, 36.61% yield) as a colourless oil.

LCMS: m/z 766.9 [M+1]⁺. ¹HNMR: (400 MHz, CHLOROFORM-d) δ ppm 4.08-3.94 (m, 6H), 3.46-3.40 (m, 1H), 2.32-2.23 (m, 6H), 2.21 (s, 6H), 1.67-1.46 (m, 15H), 1.44-1.25 (m, 47H), 0.93-0.85 (m, 12H).

### Synthesis of Lipid 21

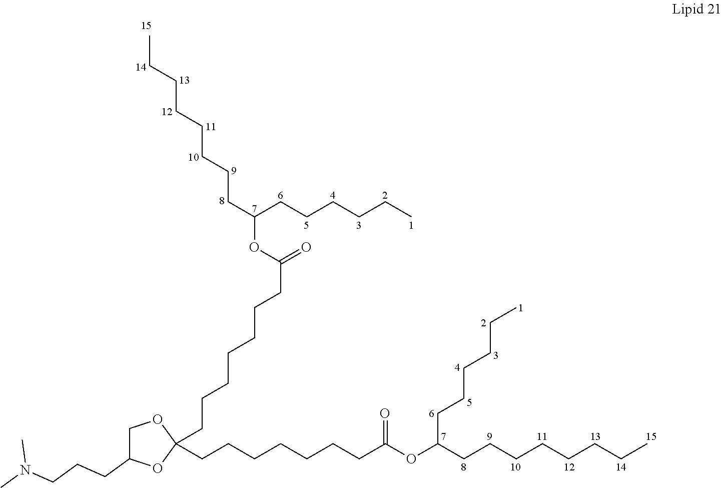 [SMILES: CCCCCCCCC(CCCCCC)OC(=O)CCCCCCCC1(CCCCCCCC(=O)OC(CCCCCC)CCCCCCCC)OCC(CCCN(C)C)O1]

A scheme for the synthesis of Lipid 21 is provided in Scheme 20 below.

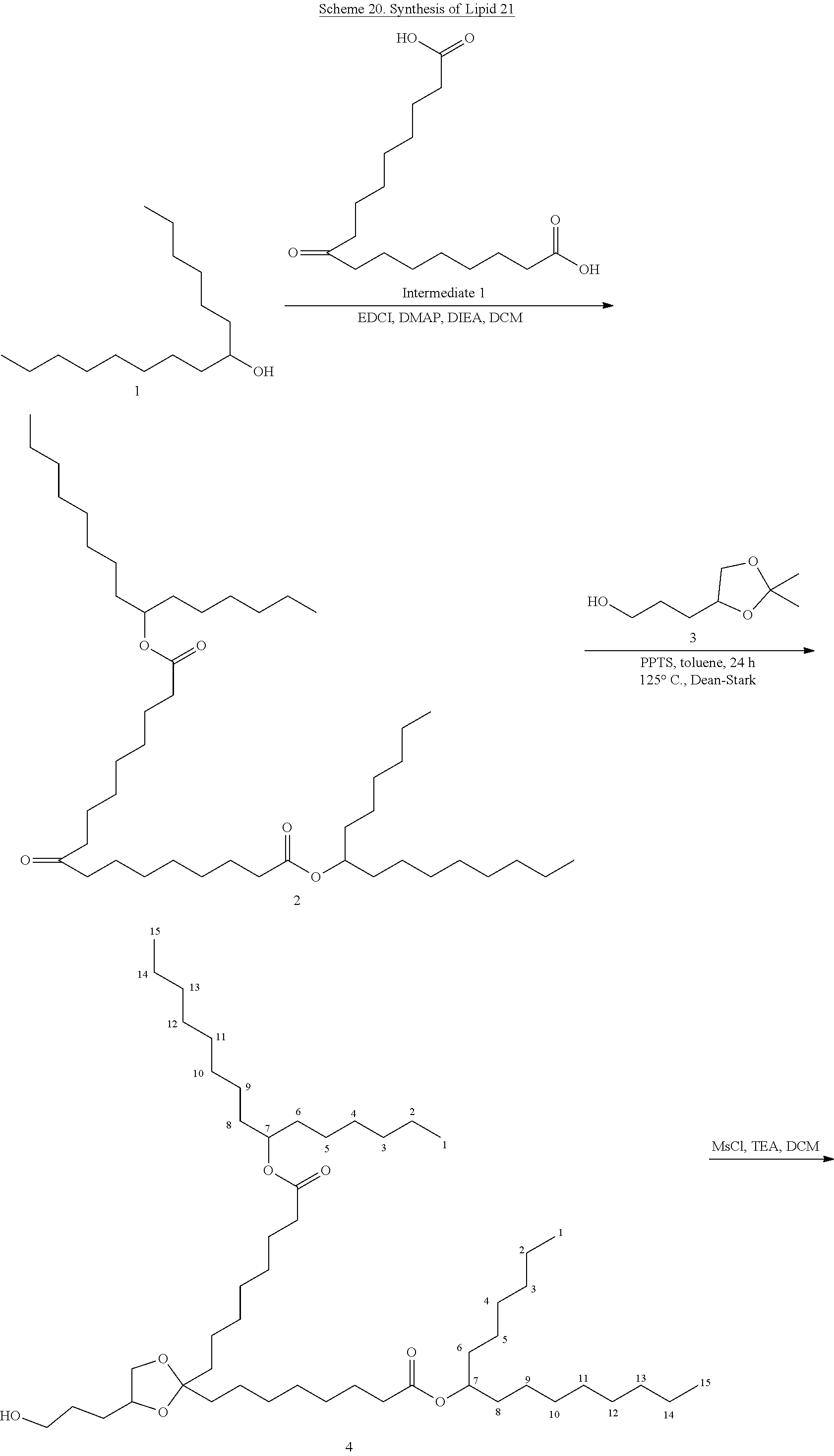

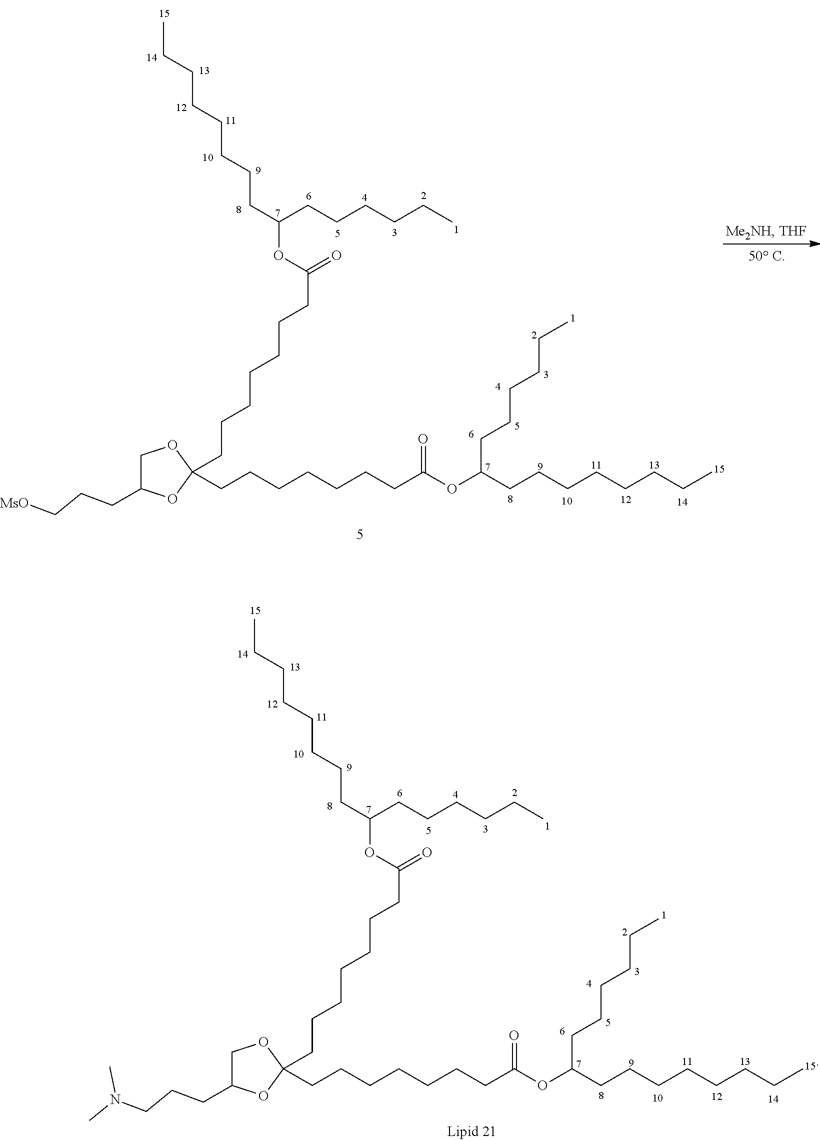

### Procedure for Preparation of Compound 3

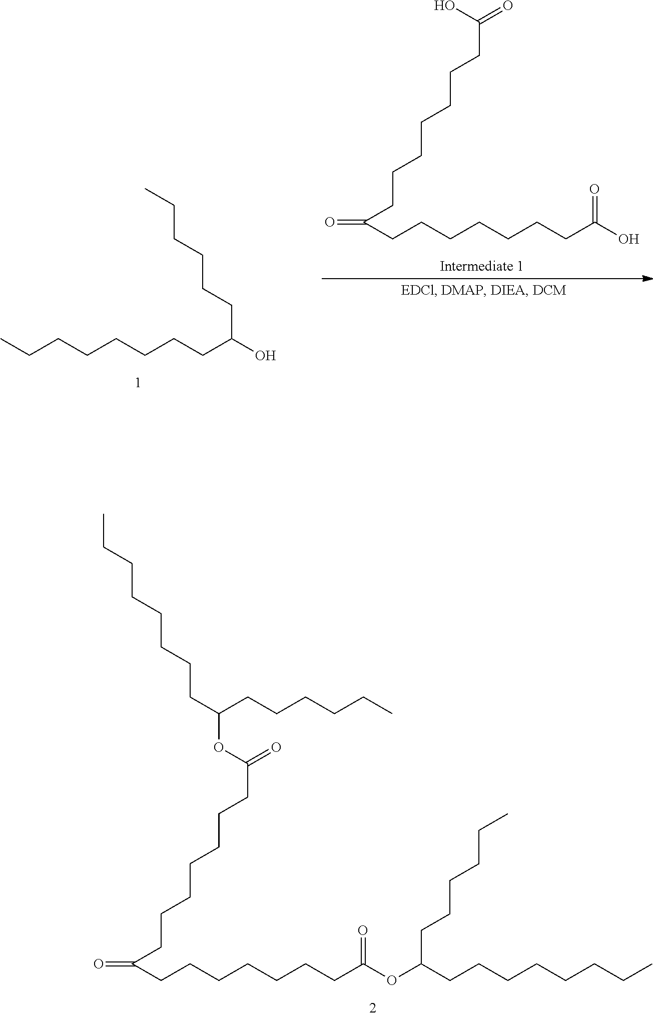 [SMILES: CCCCCCCCC(O)CCCCCC ; O=C(O)CCCCCCCC(=O)CCCCCCCC(=O)O ; CCCCCCCCC(CCCCCC)OC(=O)CCCCCCCC(=O)CCCCCCCC(=O)OC(CCCCCC)CCCCCCCC]

To a mixture of compound 1 (915.35 mg, 4.01 mmol, 2.1 eq.) and intermediate 1 (600 mg, 1.91 mmol, 1 eq.) in DCM (10 mL) was added EDCI (1.83 g, 9.54 mmol, 5 eq.), DMAP (116.57 mg, 954.15 μmol, 0.5 eq.) and DIEA (1.23 g, 9.54 mmol, 1.66 mL, 5 eq.). The mixture was stirred at 20° C. for 12 hours. The mixture was diluted with H₂O (20 mL) and extracted with DCM (10 mL*2). The combined organic layers were washed with brine (10 mL), dried over anhydrous Na₂SO₄, filtered and concentrated under reduced pressure. The residue was purified by column chromatography (SiO₂, Petroleum ether/Ethyl acetate=I/O to 100/1) twice to give compound 3 (860 mg, 71.67% yield) as a colorless oil.

¹HNMR: (400 MHz, CHLOROFORM-d) δ ppm 4.86 (quin, J=6.2 Hz, 2H), 2.37 (t, J=7.4 Hz, 4H), 2.27 (t, J=7.4 Hz, 4H), 1.66-1.47 (m, 16H), 1.33-1.22 (m, 52H), 0.88 (t, J=6.8 Hz, 12H)

### Procedure for Preparation of Compound 4

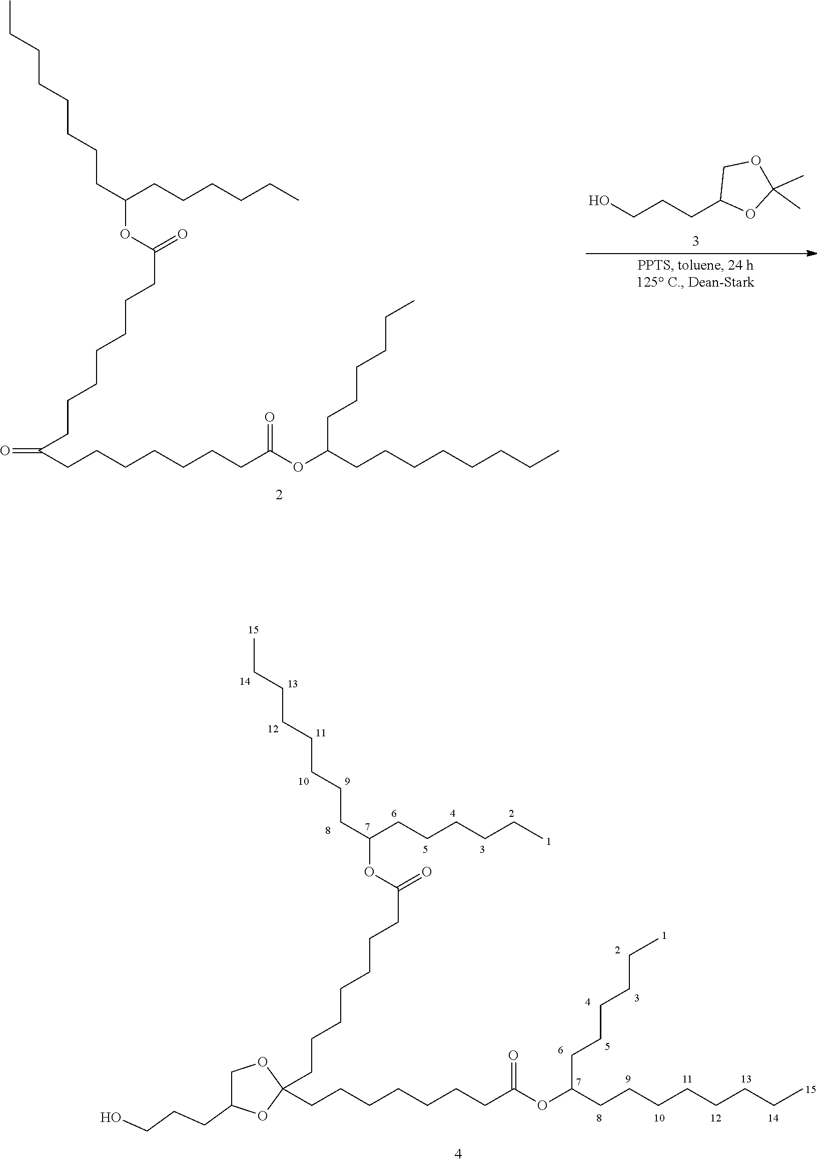 [SMILES: CCCCCCCCC(CCCCCC)OC(=O)CCCCCCCC(=O)CCCCCCCC(=O)OC(CCCCCC)CCCCCCCC ; CC1(C)OCC(CCCO)O1 ; CCCCCCCCC(CCCCCC)OC(=O)CCCCCCCC1(CCCCCCCC(=O)OC(CCCCCC)CCCCCCCC)OCC(CCCO)O1]

To a mixture of compound 2 (600 mg, 816.09 μmol, 1 eq.) and compound 3 (326.87 mg, 2.04 mmol, 2.5 eq.) in toluene (20 mL) was added PPTS (61.53 mg, 244.83 μmol, 0.3 eq.) at 25° C. The mixture was stirred at 125° C. for 12 hours equipped with Dean-Stark. The mixture was diluted with H₂O (10 mL) and extracted with DCM (20 mL*2). The combined organic layers were washed with brine (20 mL), dried over anhydrous Na₂SO₄, filtered and concentrated under reduced pressure. The residue was purified by column chromatography (SiO₂, PE:DCM=3:2/EA=100/1 to 5/1) to give compound 4 (590 mg, 86.34% yield) as a colorless oil.

¹HNMR: (400 MHz, CHLOROFORM-d) δ ppm 4.86 (quin, J=6.2 Hz, 2H), 4.15-4.02 (m, 2H), 3.76-3.63 (m, 2H), 3.46 (m, J=7.2 Hz, 1H), 2.27 (m, J=7.6 Hz, 4H), 2.19-2.10 (m, 1H), 1.71-1.47 (m, 20H), 1.37-1.20 (m, 57H), 0.87 (t, J=6.8 Hz, 12H).

### Procedure for Preparation of Compound 5

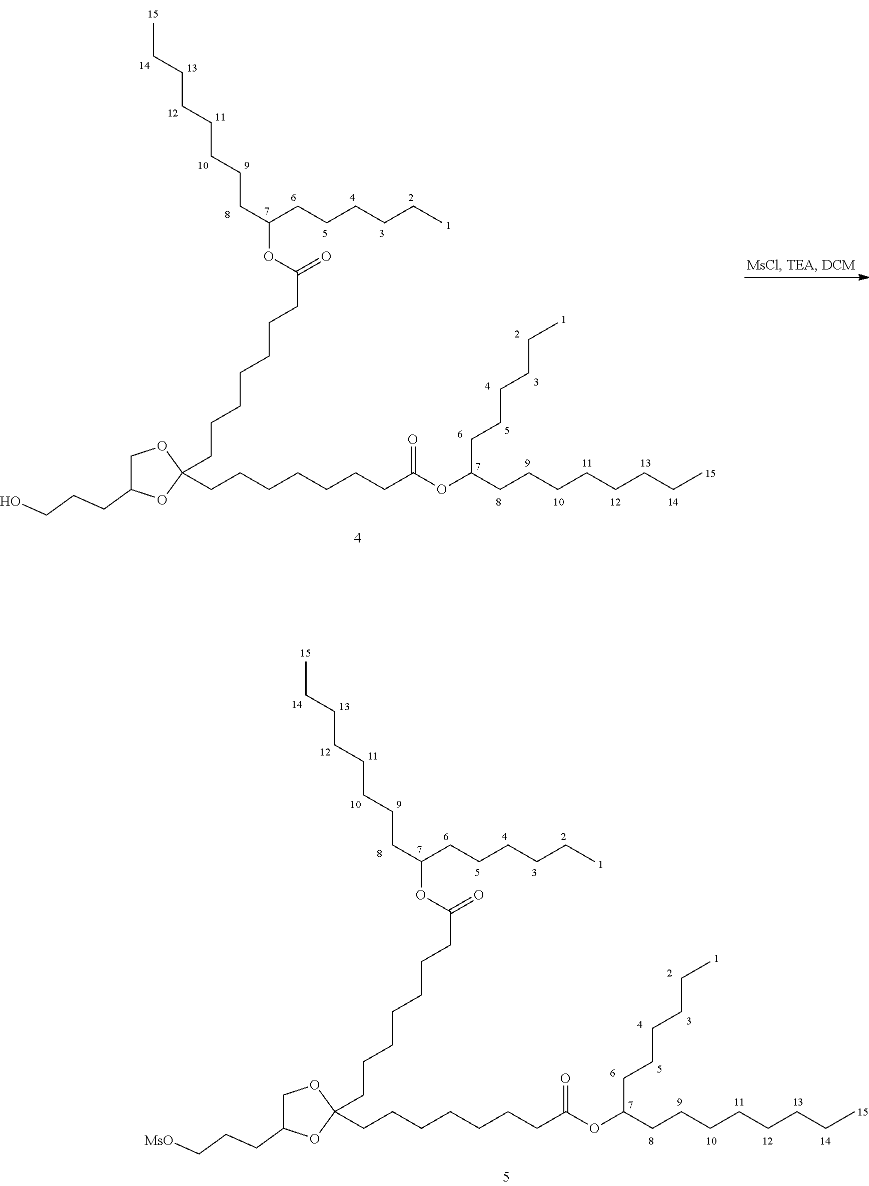 [SMILES: CCCCCCCCC(CCCCCC)OC(=O)CCCCCCCC1(CCCCCCCC(=O)OC(CCCCCC)CCCCCCCC)OCC(CCCO)O1 ; CCCCCCCCC(CCCCCC)OC(=O)CCCCCCCC1(CCCCCCCC(=O)OC(CCCCCC)CCCCCCCC)OCC(CCCOS(C)(=O)=O)O1]

To a solution of compound 4 (588 mg, 702.22 μmol, 1 eq.) in DCM (10 mL) was added TEA (588 mg, 5.81 mmol, 808.80 μL, 8.28 eq.) and MsCl (241.32 mg, 2.11 mmol, 163.05 μL, 3 eq.) at 0° C. The mixture was stirred at 25° C. for 2 hours. The mixture was poured into ice-water (10 mL) and extracted with DCM (10 mL*3). The combined organic layers were washed with brine (10 mL), dried over anhydrous Na₂SO₄, filtered and concentrated under reduced pressure to give compound 5 (510 mg, crude) as a yellow oil, which was used directly for next step without further purification.

¹HNMR: (400 MHz, CHLOROFORM-d) δ ppm 4.90-4.82 (m, 2H), 4.33-4.22 (m, 2H), 4.11-4.00 (m, 2H), 3.68 (s, 1H), 3.50-3.38 (m, 2H), 3.01 (s, 3H), 2.27 (t, J=7.6 Hz, 4H), 2.03-1.78 (m, 3H), 1.54-1.46 (m, 8H), 1.71-1.46 (m, 1H), 1.45-1.37 (m, 3H), 1.32-1.22 (m, 57H), 0.89-0.84 (m, 12H).

### Procedure for Preparation of Lipid 21

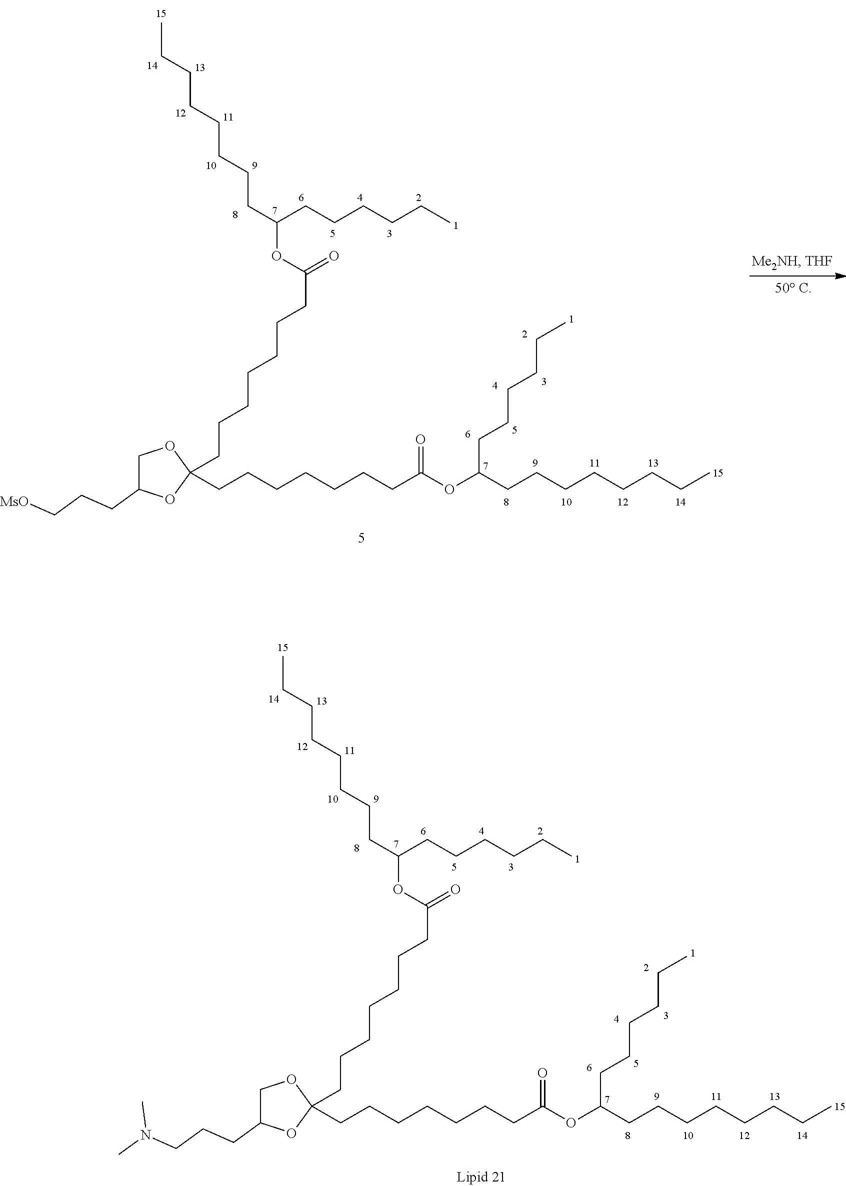

A mixture of compound 5 (500 mg, 546.19 μmol, 1 eq.) in Me₂NH (2 M THE solution, 8 mL) was stirred at 50° C. for 12 hours. The reaction mixture was concentrated under reduced pressure. The residue was purified by column chromatography (SiO₂, PE/EA=10/1 to 5/1, eluent with 0.1% TEA), followed by another round column purification (SiO₂, A/B=100/1 to 5/1, phase A: PE:DCM=3:1, phase B: EtOAc, eluent with 0.1% TEA), and then further purified by prep-NPLC {column: Welch Ultimate XB—SiOH 250*50*10 μm; mobile phase: [Hexane-EtOH]; B %: 1%, isocratic elution mode} to give Lipid 21 (76.2 mg, 66.67% yield) as a colorless oil.

LCMS: m/z: 865.1 [M+1]⁺. ¹HNMR: (400 MHz, CHLOROFORM-d) δ ppm 4.87 (m, J=6.2 Hz, 2H), 4.10-4.01 (m, 2H), 3.49-3.43 (m, 1H), 2.28 (t, J=7.6 Hz, 6H), 2.23 (s, 6H), 1.66-1.48 (m, 25H), 1.35-1.21 (m, 60H), 0.91-0.86 (m, 12H).

### Synthesis of Lipid 23

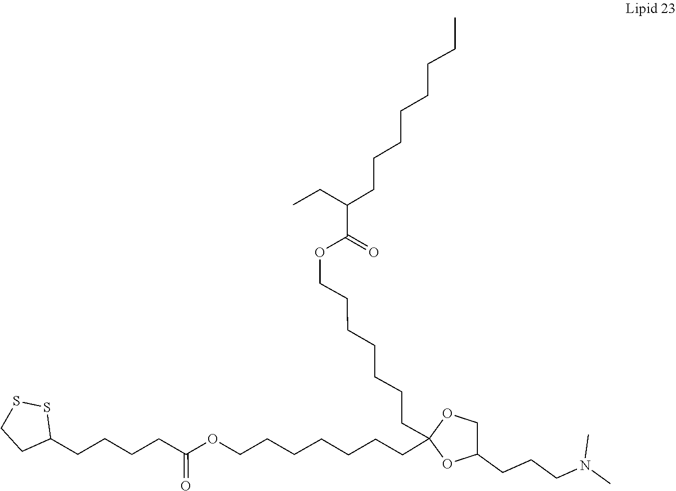 [SMILES: CCCCCCCCC(CC)C(=O)OCCCCCCCC1(CCCCCCCOC(=O)CCCCC2CCSS2)OCC(CCCN(C)C)O1]

A scheme for the synthesis of Lipid 23 is provided in Scheme 21 below.

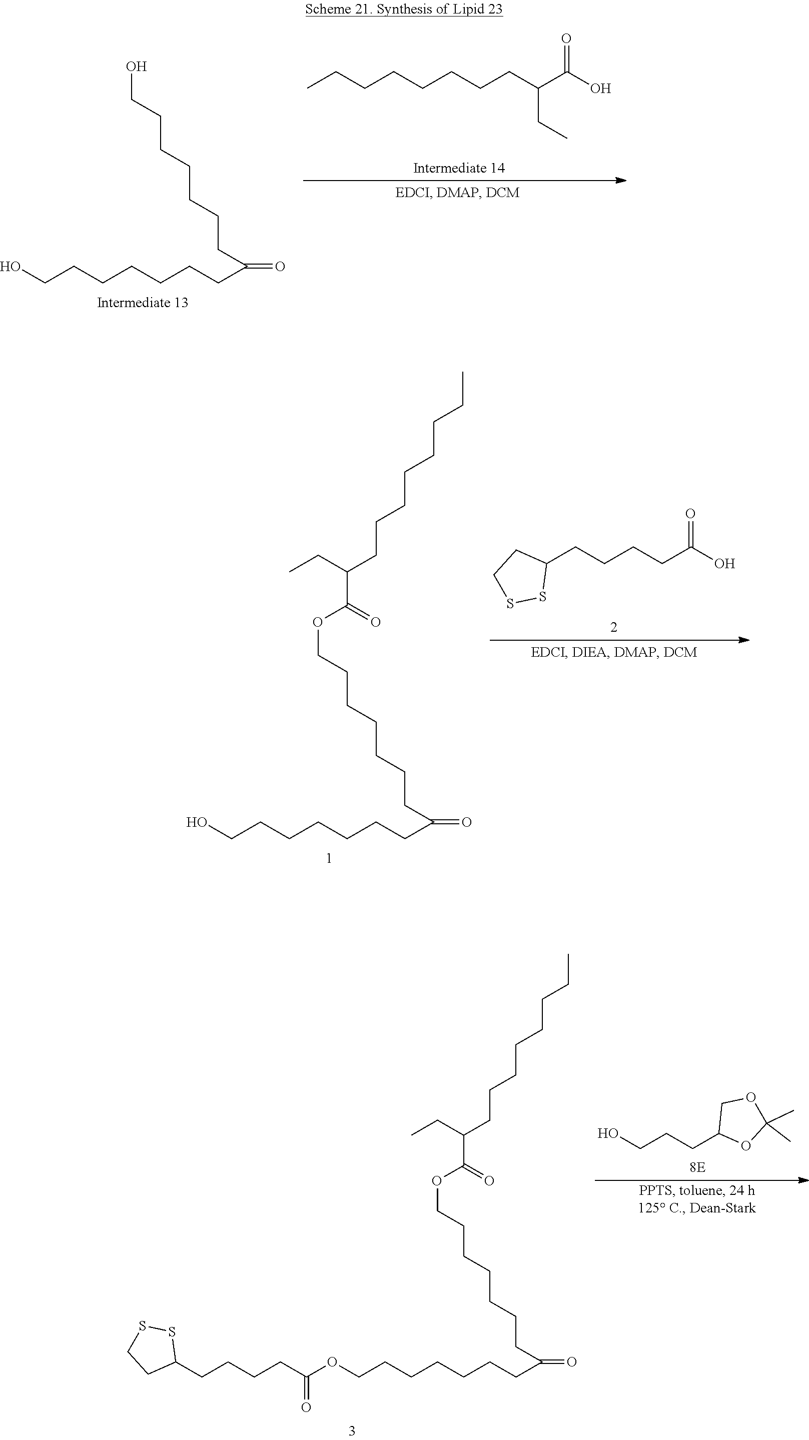 [SMILES: O=C(CCCCCCCO)CCCCCCCO ; CCCCCCCCC(CC)C(=O)O ; CCCCCCCCC(CC)C(=O)OCCCCCCCC(=O)CCCCCCCO ; O=C(O)CCCCC1CCSS1 ; CCCCCCCCC(CC)C(=O)OCCCCCCCC(=O)CCCCCCCOC(=O)CCCCC1CCSS1 ; CC1(C)OCC(CCCO)O1]

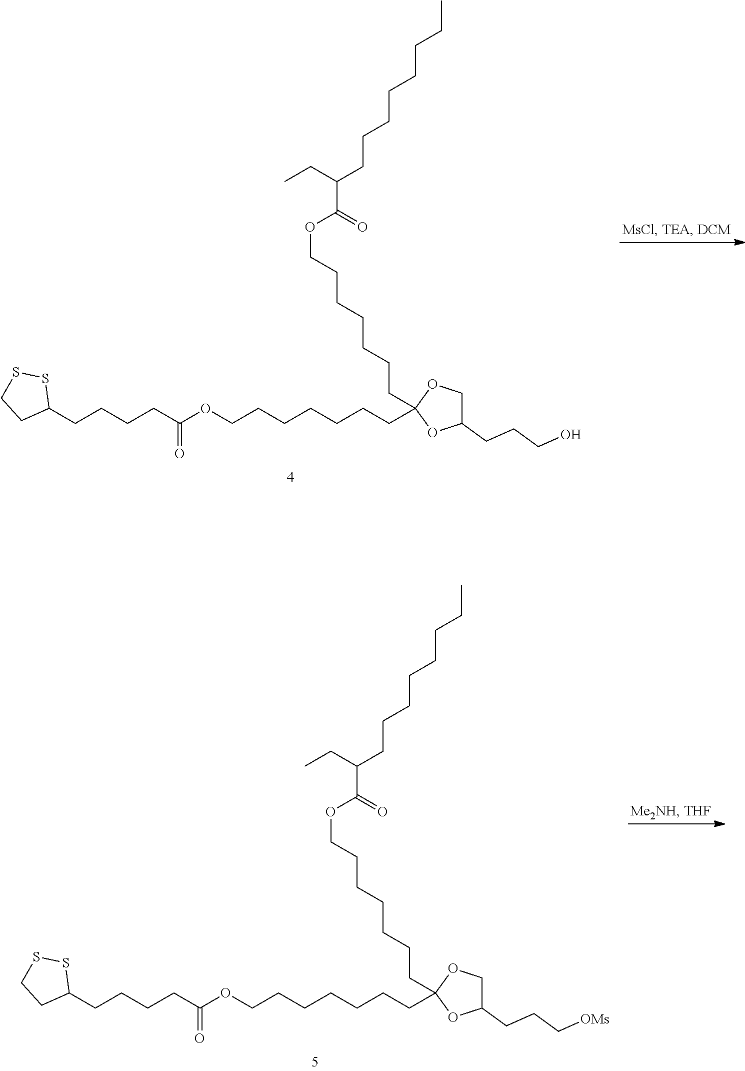 [SMILES: CCCCCCCCC(CC)C(=O)OCCCCCCCC1(CCCCCCCOC(=O)CCCCC2CCSS2)OCC(CCCO)O1 ; CCCCCCCCC(CC)C(=O)OCCCCCCCC1(CCCCCCCOC(=O)CCCCC2CCSS2)OCC(CCCOS(C)(=O)=O)O1]

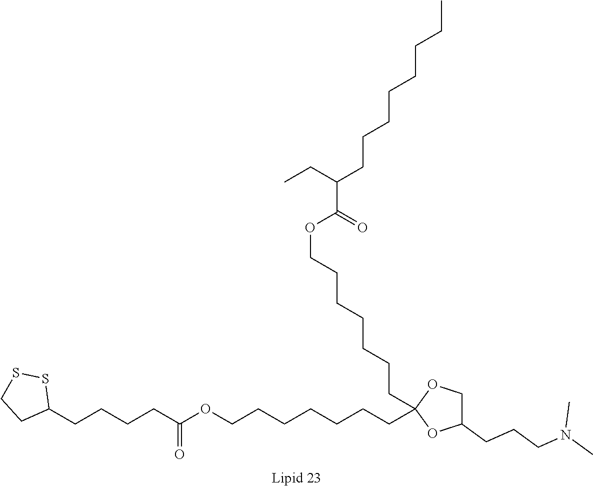 [SMILES: CCCCCCCCC(CC)C(=O)OCCCCCCCC1(CCCCCCCOC(=O)CCCCC2CCSS2)OCC(CCCN(C)C)O1]

### Procedure for Preparation of Compound 1

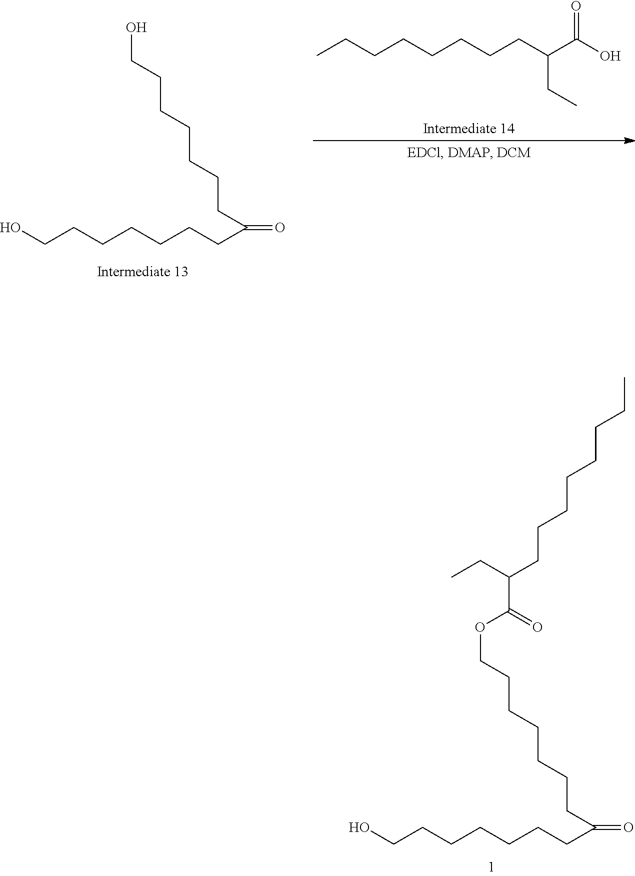 [SMILES: O=C(CCCCCCCO)CCCCCCCO ; CCCCCCCCC(CC)C(=O)O ; CCCCCCCCC(CC)C(=O)OCCCCCCCC(=O)CCCCCCCO]

To a mixture of intermediate 13 (1.5 g, 5.81 mmol, 1.5 eq.) and intermediate 14 (775.23 mg, 3.87 mmol, 1 eq.) in DCM (25 mL) was added EDCI (971.87 mg, 5.07 mmol, 1.31 eq.) and DMAP (486.97 mg, 3.99 mmol, 1.03 eq.). The mixture was stirred at 25° C. for 12 hours. The mixture was poured into ice-water (w/w=1/1, 30 mL), and then extracted with DCM (30 mL*3). The combined organic phases were washed with brine (50 mL), dried over anhydrous Na₂SO₄, filtered and concentrated in vacuo. The residue was purified by column chromatography (SiO₂, Petroleum ether/Ethyl acetate=5/1 to 3/1) to give compound 1 (700 mg, 41.04% yield) as a colorless oil. (One spot on TLC but not very clean on HNMR)

¹HNMR: (400 MHz, CHLOROFORM-d) δ ppm 4.11-4.02 (m, 3H), 3.64 (t, J=6.4 Hz, 2H), 2.39 (t, J=7.2 Hz, 4H), 2.33-2.19 (m, 1H), 1.68-1.42 (m, 12H), 1.41-1.20 (m, 36H), 0.94-0.83 (m, 6H).

### Procedure for Preparation of Compound 3

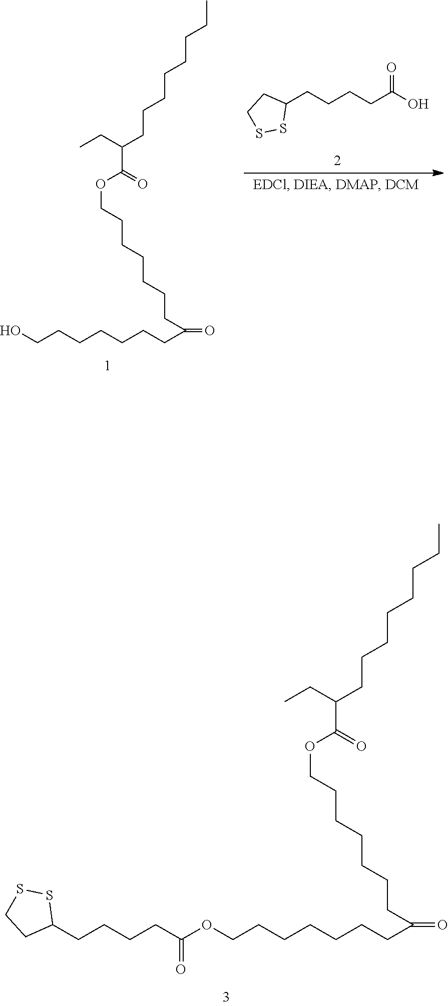 [SMILES: CCCCCCCCC(CC)C(=O)OCCCCCCCC(=O)CCCCCCCO ; O=C(O)CCCCC1CCSS1 ; CCCCCCCCC(CC)C(=O)OCCCCCCCC(=O)CCCCCCCOC(=O)CCCCC1CCSS1]

To a mixture of compound 1 (600 mg, 1.36 mmol, 1 eq.), compound 2 (360 mg, 1.74 mmol, 1.28 eq.), DIEA (720 mg, 5.57 mmol, 970.35 μL, 4.09 eq.) and DMAP (168 mg, 1.38 mmol, 1.01 eq.) in DCM (3 mL) was added EDCI (780.00 mg, 4.07 mmol, 2.99 eq.) at 25° C. and the mixture was stirred at 25° C. for 12 hours. The reaction mixture was concentrated under reduced pressure. The residue was purified by silica gel column (SiO₂, Petroleum ether/Ethyl acetate=50/1 to 5/1 to 1/1) to give compound 3 (350 mg, 40.87% yield) as a colourless oil.

¹HNMR: (400 MHz, CHLOROFORM-d) δ ppm 4.15-3.95 (m, 4H), 3.63-3.51 (m, 1H), 3.26-3.07 (m, 2H), 2.52-2.20 (m, 8H), 1.92 (dd, J=6.4, 13.2 Hz, 1H), 1.76-1.21 (m, 42H), 0.94-0.82 (m, 6H).

### Procedure for Preparation of Compound 4

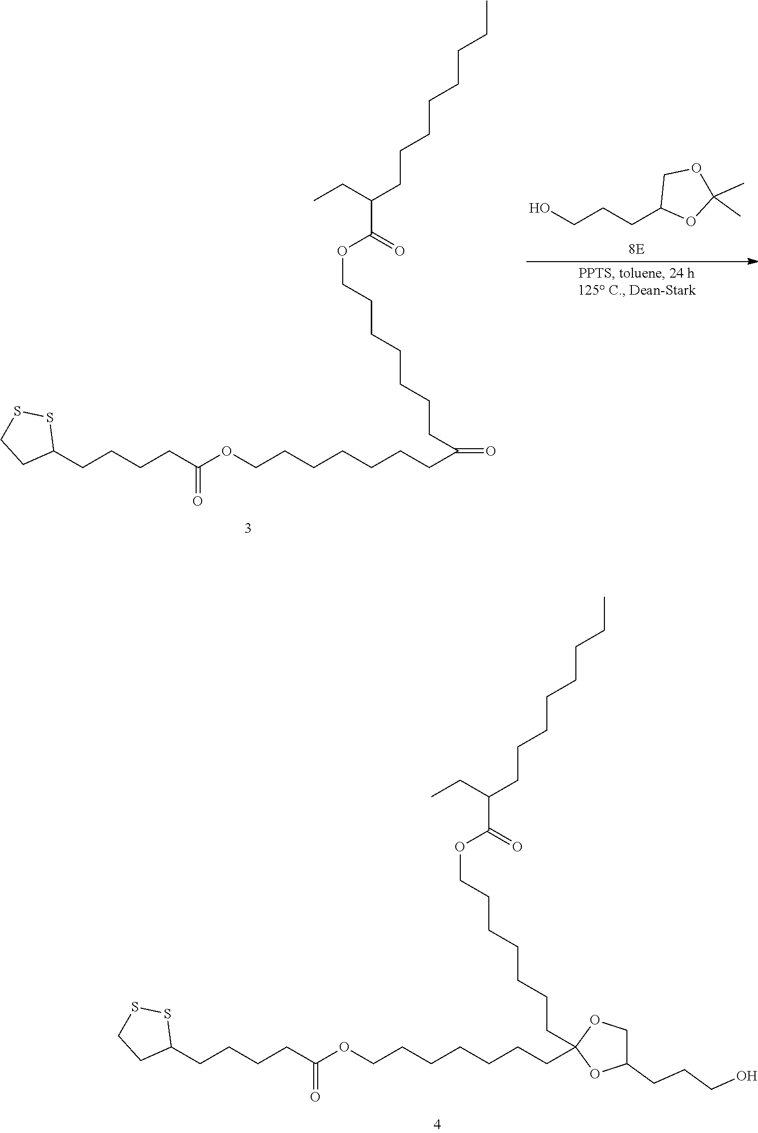 [SMILES: CCCCCCCCC(CC)C(=O)OCCCCCCCC(=O)CCCCCCCOC(=O)CCCCC1CCSS1 ; CC1(C)OCC(CCCO)O1 ; CCCCCCCCC(CC)C(=O)OCCCCCCCC1(CCCCCCCOC(=O)CCCCC2CCSS2)OCC(CCCO)O1]

To a mixture of compound 3 (350 mg, 556.43 μmol, 1 eq.) and compound 8E (222.87 mg, 1.39 mmol, 2.5 eq.) in toluene (10 mL) was added PPTS (69.92 mg, 278.22 μmol, 0.5 eq.) at 25° C. and the mixture was stirred at 125° C. for 12 hours equipped with Dean-Stark. The reaction mixture was poured into ice-water (20 mL), and then extracted with DCM (10 mL*3). The combined organic layers were washed with brine (10 mL), dried over anhydrous Na₂SO₄, filtered and concentrated under reduced pressure. The crude product was purified by column chromatography (SiO₂, Petroleum ether/Ethyl acetate=100/1 to 10:1 to 3:1) to give compound 4 (350 mg, 78.75% yield, >90% purity) as a yellow oil.

¹HNMR: (400 MHz, CHLOROFORM-d) δ ppm 4.18-4.01 (m, 8H), 3.76-3.64 (m, 4H), 3.61-3.52 (m, 2H), 3.47 (t, J=7.2 Hz, 1H), 3.28-3.02 (m, 2H), 2.47 (dd, J=6.4, 12.4 Hz, 1H), 2.36-2.21 (m, 3H), 1.98-1.87 (m, 2H), 1.75-1.21 (m, 65H), 0.94-0.84 (m, 6H).

### Procedure for Preparation of Compound 5

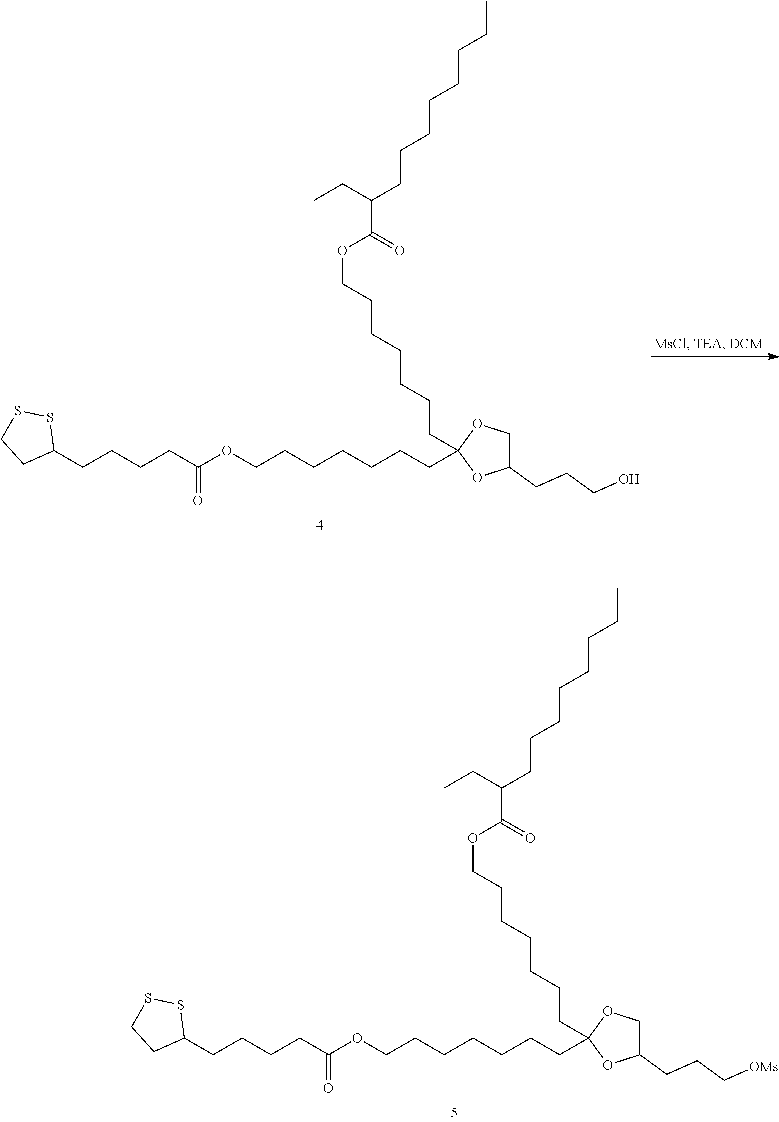 [SMILES: CCCCCCCCC(CC)C(=O)OCCCCCCCC1(CCCCCCCOC(=O)CCCCC2CCSS2)OCC(CCCO)O1 ; CCCCCCCCC(CC)C(=O)OCCCCCCCC1(CCCCCCCOC(=O)CCCCC2CCSS2)OCC(CCCOS(C)(=O)=O)O1]

To a mixture of compound 4 (350 mg, 478.71 μmol, 1 eq.) and TEA (242.20 mg, 2.39 mmol, 333.15 μL, 5 eq.) in DCM (3 mL) was added MsCl (200 mg, 1.75 mmol, 135.14 μL, 3.65 eq.) at 5° C., and then the mixture was stirred at 25° C. for 2 hours. The reaction mixture was poured into ice-water (20 mL), and then extracted with DCM (10 mL*3). The combined organic layers were washed with brine (10 mL), dried over anhydrous Na₂SO₄, filtered and concentrated under vacuum to give compound 5 (350 mg, 90.35% yield) as a yellow oil.

¹HNMR: (400 MHz, CHLOROFORM-d) δ ppm 4.33-4.25 (m, 4H), 4.17-4.11 (m, 1H), 4.08-4.05 (m, 4H), 3.50-3.45 (m, 1H), 3.24-3.09 (m, 5H), 2.52-2.41 (m, 1H), 2.37-2.27 (m, 2H), 2.02-1.78 (m, 6H), 1.75-1.59 (m, 15H), 1.54-1.44 (m, 4H), 1.38-1.23 (m, 30H), 0.89 (dt, J=3.2, 7.2 Hz, 6H).

### Procedure for Preparation of Lipid 23

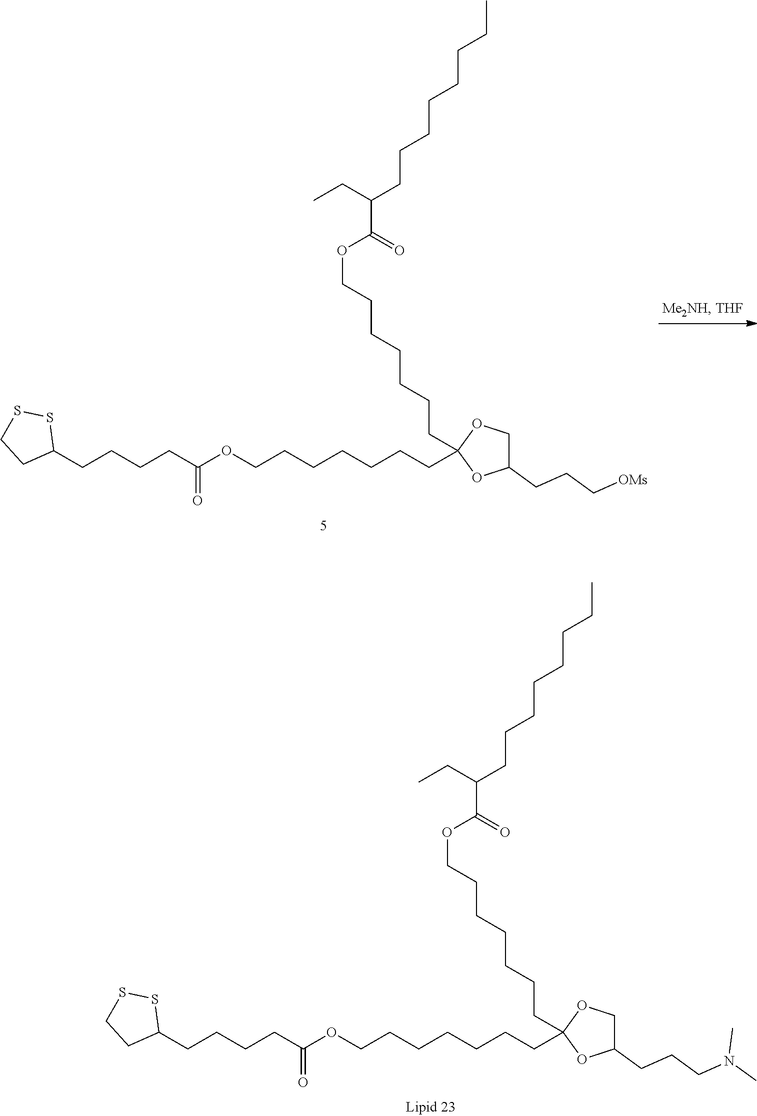 [SMILES: CCCCCCCCC(CC)C(=O)OCCCCCCCC1(CCCCCCCOC(=O)CCCCC2CCSS2)OCC(CCCOS(C)(=O)=O)O1 ; CCCCCCCCC(CC)C(=O)OCCCCCCCC1(CCCCCCCOC(=O)CCCCC2CCSS2)OCC(CCCN(C)C)O1]

A solution of compound 5 (350 mg, 432.51 μmol, 1 eq.) in Me₂NH (2 M THF solution, 20 mL, 92.48 eq.) was stirred at 50° C. for 12 hours. The reaction mixture was concentrated under reduced pressure. The crude product was purified by column chromatography (SiO₂, A/B=32/1 to 13/1: A=PE/DCM (v/v=5/1), B=EtOAc, 0.1% TEA added to B) twice to give Lipid 23 (180 mg, 53.24% yield, 97% purity) as a yellow oil.

Special LCMS: m/z, 758.7 [M+1]⁺. ¹HNMR: (400 MHz, CHLOROFORM-d) δ ppm 4.16-3.99 (m, 6H), 3.64-3.52 (m, 1H), 3.46 (t, J=7.2, 1H), 3.25-3.07 (m, 2H), 2.54-2.42 (m, 1H), 2.36-2.26 (m, 4H), 2.23 (s, 6H), 1.97-1.85 (m, 1H), 1.75-1.21 (m, 50H), 0.89 (dt, J=3.2, 7.2 Hz, 6H).

### Synthesis of Lipid 24 and Lipid 28

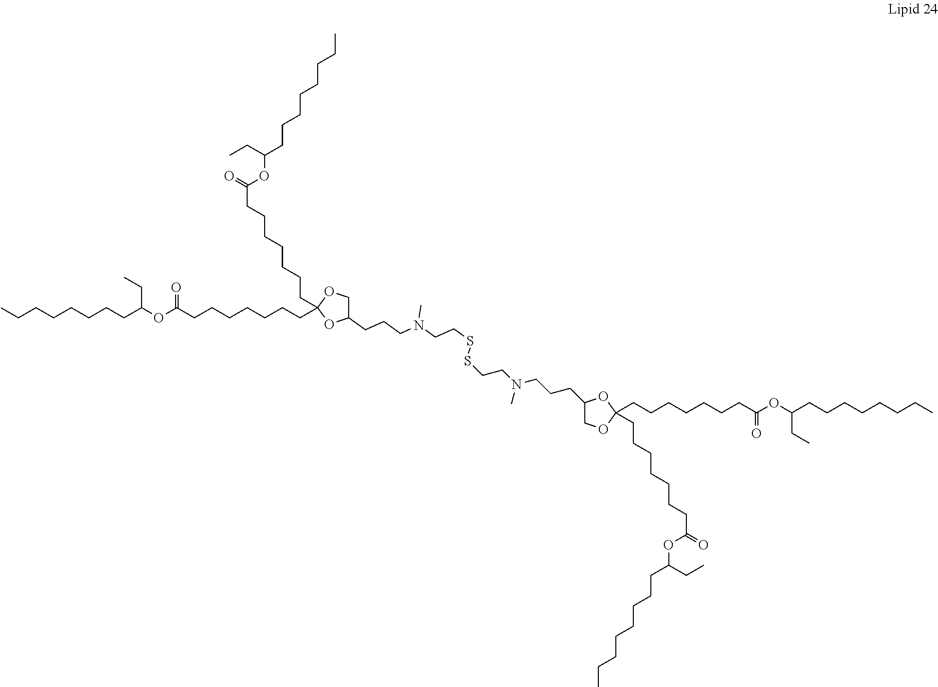 [SMILES: CCCCCCCCC(CC)OC(=O)CCCCCCCC1(CCCCCCCC(=O)OC(CC)CCCCCCCC)OCC(CCCN(C)CCSSCCN(C)CCCC2COC(CCCCCCCC(=O)OC(CC)CCCCCCCC)(CCCCCCCC(=O)OC(CC)CCCCCCCC)O2)O1]

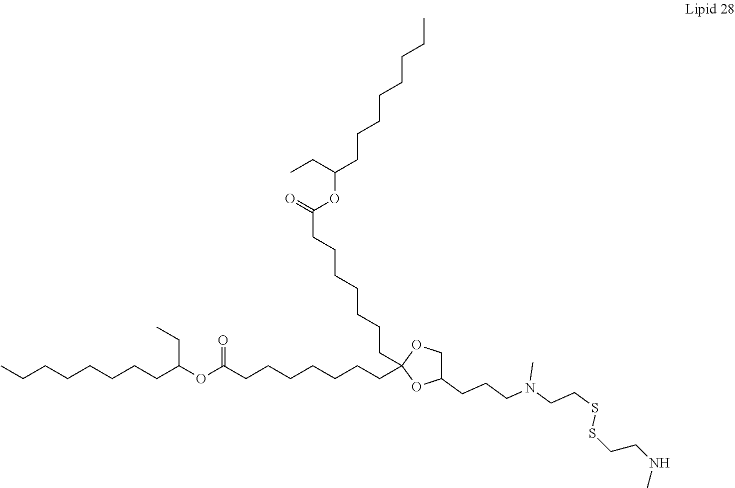 [SMILES: CCCCCCCCC(CC)OC(=O)CCCCCCCC1(CCCCCCCC(=O)OC(CC)CCCCCCCC)OCC(CCCN(C)CCSSCCNC)O1]

A scheme for the synthesis of Lipid 24 and Lipid 28 is provided in Scheme 22 below.

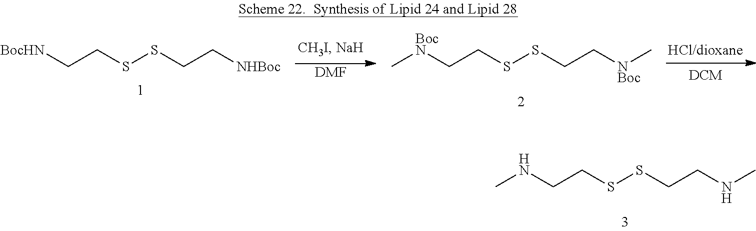

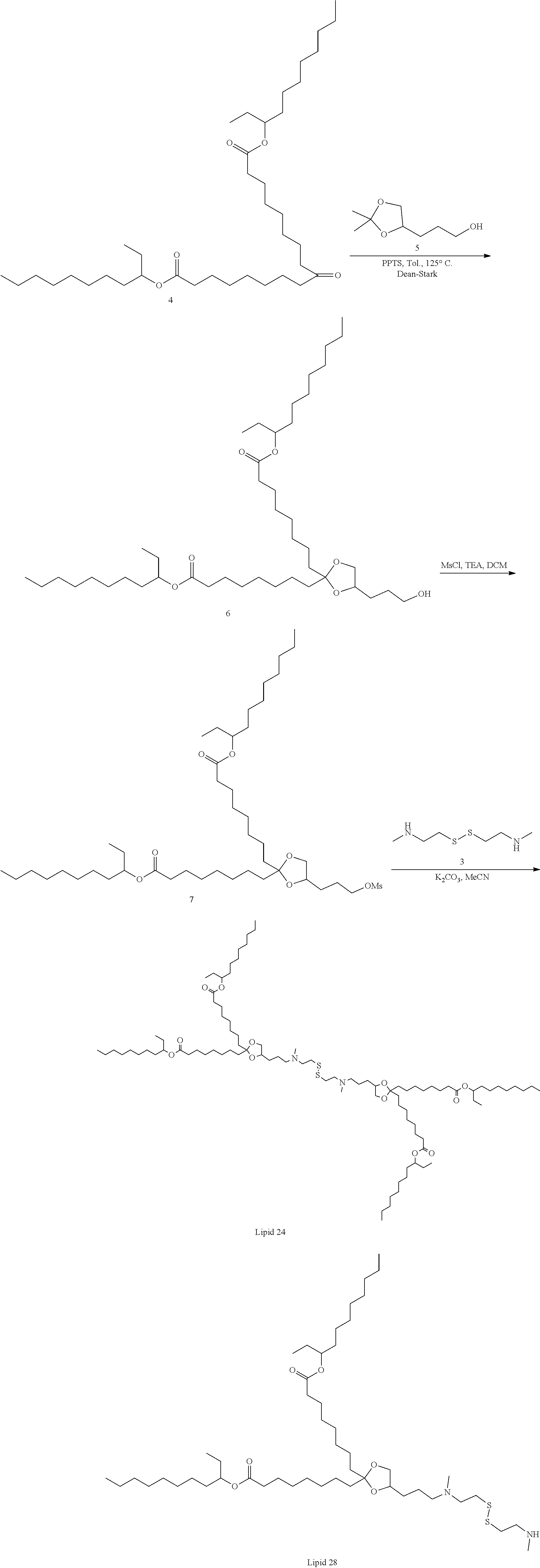

### Procedure for Preparation of Compound 2

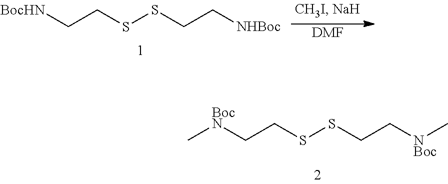

To a solution of compound 1 (5 g, 14.18 mmol, 1 eq.) in DMF (60 mL) was added NaH (1.25 g, 31.20 mmol, 60% purity, 2.2 eq.) and CH₃I (4.83 g, 34.04 mmol, 2.12 mL, 2.4 eq.) alternately at 0° C. The mixture was stirred at 25° C. for 2 hours. The mixture was poured into water (50 mL) at 0° C. and extracted with EtOAc (50 mL*2). The combined organic layers were washed with brine (30 mL*2), dried over anhydrous Na₂SO₄, filtered and concentrated in vacuo. The residue was purified by column chromatography (SiO₂, Petroleum ether/Ethyl acetate=I/O to 3/1) to give compound 2 (3.04 g, 56.32% yield) as a colorless oil.

¹H NMR (400 MHz, CHLOROFORM-d) δ 3.50 (br s, 4H), 2.89 (s, 6H), 2.82 (br s, 4H), 1.46 (s, 18H).

### Procedure for Preparation of Compound 3

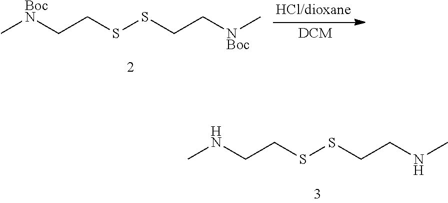

To a solution of compound 2 (3 g, 7.88 mmol, 1 eq.) in DCM (15 mL) was added HCl/dioxane (2 M, 15 mL, 3.81 eq.) at 25° C. The mixture was stirred at 25° C. for 2 hours. The reaction mixture was concentrated under reduced pressure and dried in vacuo to give crude compound 3 (1.985 g, 2HCl salt) as a white solid.

¹H NMR (400 MHz, DMSO-d₆) δ 9.45-9.26 (m, 4H), 3.24 (br s, 4H), 3.18-3.10 (m, 4H), 2.60 (br d, J=4.8 Hz, 6H).

### Procedure for Preparation of Compound 6

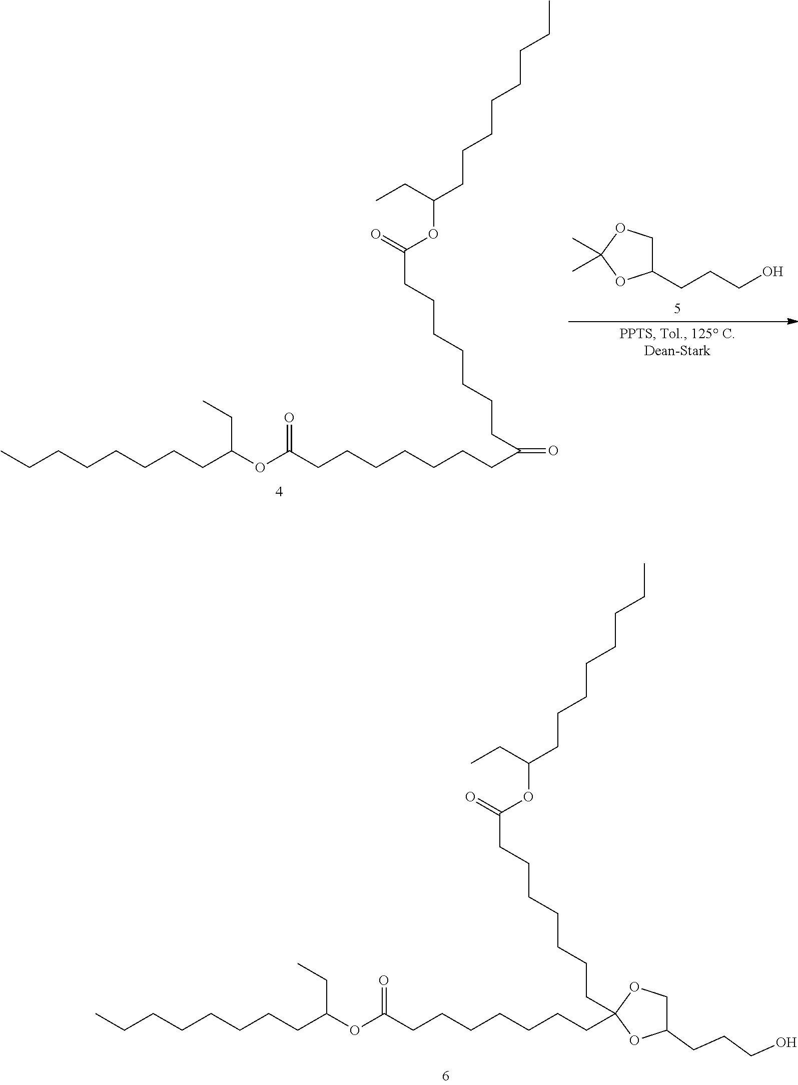 [SMILES: CCCCCCCCC(CC)OC(=O)CCCCCCCC(=O)CCCCCCCC(=O)OC(CC)CCCCCCCC ; CC1(C)OCC(CCCO)O1 ; CCCCCCCCC(CC)OC(=O)CCCCCCCC1(CCCCCCCC(=O)OC(CC)CCCCCCCC)OCC(CCCO)O1]

To a solution of compound 4 (2 g, 3.21 mmol, 1 eq.) and compound 5 (1.29 g, 8.03 mmol, 2.5 eq.) in toluene (20 mL) was added PPTS (242.02 mg, 963.08 μmol, 0.3 eq.). The mixture was stirred at 135° C. for 12 hours equipped with a Dean-Stark. The reaction mixture was quenched by addition of aq. NaHCO₃ (sat., 20 mL) at 25° C., and then diluted with H₂O (20 mL) and extracted with EtOAc (30 mL*2). The combined organic layers were washed with brine (10 mL*2), dried over anhydrous Na₂SO₄, filtered and concentrated under reduced pressure. The residue was purified by column chromatography (SiO₂, Petroleum ether/Ethyl acetate=I/O to 3/1) to give compound 6 (1.8 g, 77.32% yield) as a colorless oil.

¹H NMR (400 MHz, CHLOROFORM-d) δ 4.81 (quin, J=6.4 Hz, 2H), 4.13-4.03 (m, 2H), 3.73-3.64 (m, 2H), 3.50-3.43 (m, 1H), 2.28 (t, J=7.6 Hz, 4H), 1.70-1.47 (m, 20H), 1.37-1.22 (m, 40H), 0.91-0.84 (m, 12H).

### Procedure for Preparation of Compound 7

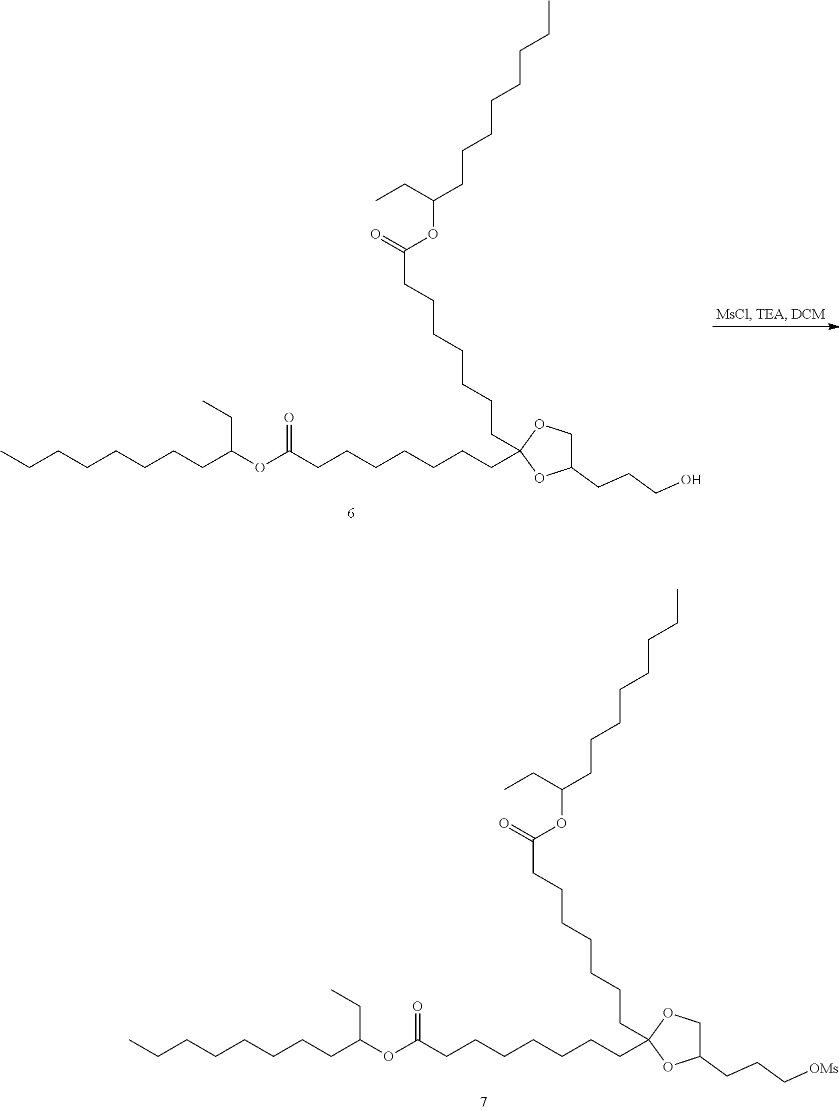 [SMILES: CCCCCCCCC(CC)OC(=O)CCCCCCCC1(CCCCCCCC(=O)OC(CC)CCCCCCCC)OCC(CCCO)O1 ; CCCCCCCCC(CC)OC(=O)CCCCCCCC1(CCCCCCCC(=O)OC(CC)CCCCCCCC)OCC(CCCOS(C)(=O)=O)O1]

To a solution of compound 6 (1.8 g, 2.48 mmol, 1 eq.) in DCM (20 mL) was added MsCl (853.05 mg, 7.45 mmol, 576.38 μL, 3 eq.) and TEA (1.26 g, 12.41 mmol, 1.73 mL, 5 eq.) at 0° C. The mixture was stirred at 20° C. for 2 hours. The reaction mixture was poured into ice-water (15 mL) and extracted with DCM (20 mL*3). The combined organic layers were washed with brine (20 mL), dried over anhydrous Na₂SO₄, filtered and concentrated under reduced pressure. The residue was purified by column chromatography (SiO₂, PE/EA=10/1 to 5/1, with 0.1% TEA as modifier) to give compound 7 (1.8 g, 90.28% yield) as a colorless oil.

¹H NMR (400 MHz, CHLOROFORM-d) δ 4.85-4.78 (m, 2H), 4.34-4.24 (m, 2H), 4.16-4.03 (m, 2H), 3.50-3.44 (m, 1H), 3.02 (s, 3H), 2.29 (t, J=7.6 Hz, 4H), 1.99-1.77 (m, 2H), 1.70-1.51 (m, 18H), 1.35-1.23 (m, 40H), 0.93-0.85 (m, 12H).

### Procedure for Preparation of Lipid 24 and Lipid 28

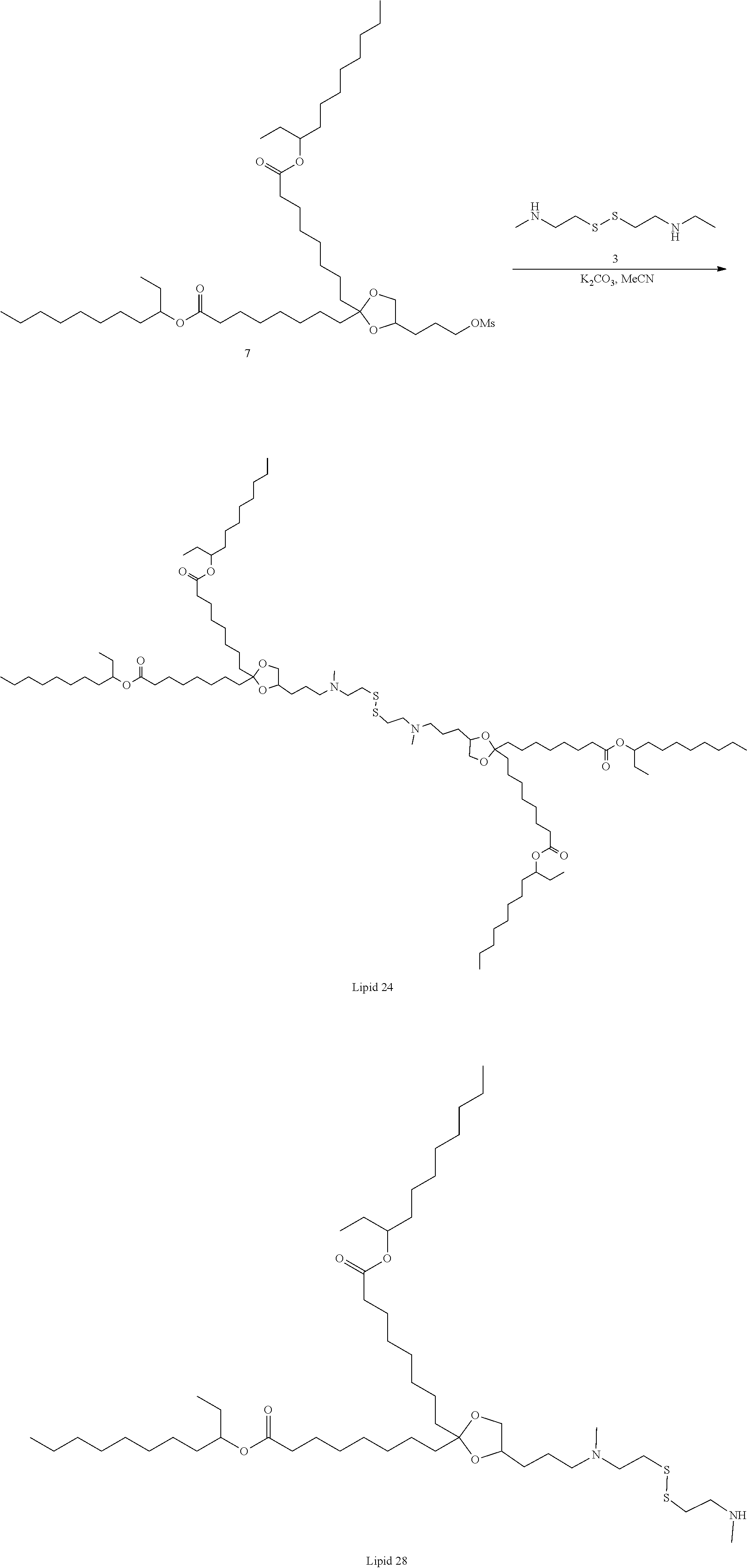

To a mixture of compound 7 (1.5 g, 1.87 mmol, 1 eq.) and K₂CO₃ (774.29 mg, 5.60 mmol, 3 eq.) in MeCN (15 mL) was added compound 3 (236.48 mg, 933.74 μmol, 0.5 eq., 2HCl salt) at 25° C., and then the mixture was stirred at 80° C. for 12 hours. The reaction mixture was filtered and the filtrate was concentrated under reduced pressure. The residue was purified by column chromatography (SIO₂, PE/EA=10/1 to 5/1, with 0.1% TEA as modifier) to give crude product 1 (Lipid 24), followed by eluting with (A/B=100/1 to 5/1, phase A: PE:DCM=3:1, phase B: MeOH, with 0.1% TEA as modifier) to give crude product 2 (Lipid 28).

The crude product 1 was further purified by column chromatography (Si₂O, DCM/acetone=100/1 to 5/1, with 0.1% TEA as modifier), followed by column chromatography (Si₂O, A/B=100/1 to 5/1, phase A: PE:DCM=3:1, phase B: Acetone, with 0.1% TEA as modifier) to give Lipid 24 (90 mg, 92% purity) and highly pure Lipid 24 (25 mg, 97.95% purity) as a yellow oil.

LCMS m/z 1594.7 [M+1]⁺. ¹H NMR (400 MHz, CHLOROFORM-d) δ 4.86-4.78 (m, 4H), 4.10-4.00 (m, 4H), 3.48˜ 3.41 (m, 2H), 2.87-2.75 (m, 4H), 2.69 (br d, J=7.6 Hz, 4H), 2.41 (br s, 4H), 2.34-2.21 (m, 14H), 1.69-1.47 (m, 44H), 1.34-1.24 (m, 76H), 0.92-0.85 (m, 24H).

The crude product 2 was further purified by prep-HPLC (PHS-Phenyl-Hexyl 150*25 mm*7 μm; mobile phase: [water (TFA)-ACN]; gradient: 48%-78% B over 15 min). The combined eluents were immediately neutralized by TEA and concentrated under reduced pressure to remove ACN, then the aqueous layer was extracted with a mixed solvent of (PE:EtOAc=3:1, 50 mL). The organic phase was washed with water (10 mL*3), dried over anhydrous Na₂SO₄, filtered and concentrated under reduced pressure to give Lipid 28 (110 mg, 96.83% purity) as a yellow oil.

LCMS m/z 887.8 [M+1]⁺. ¹H NMR (400 MHz, CHLOROFORM-d) δ 4.82 (quin, J=6.1 Hz, 2H), 4.08-4.01 (m, 2H), 3.48-3.42 (m, 1H), 2.94-2.89 (m, 2H), 2.87-2.78 (m, 4H), 2.72-2.64 (m, 2H), 2.47 (s, 3H), 2.41 (br t, J=6.8 Hz, 2H), 2.27 (br s, 4H), 2.26 (s, 3H), 1.64-1.51 (m, 18H), 1.39-1.21 (m, 42H), 0.91-0.85 (m, 12H).

### Synthesis of Lipid 25 and Lipid 29

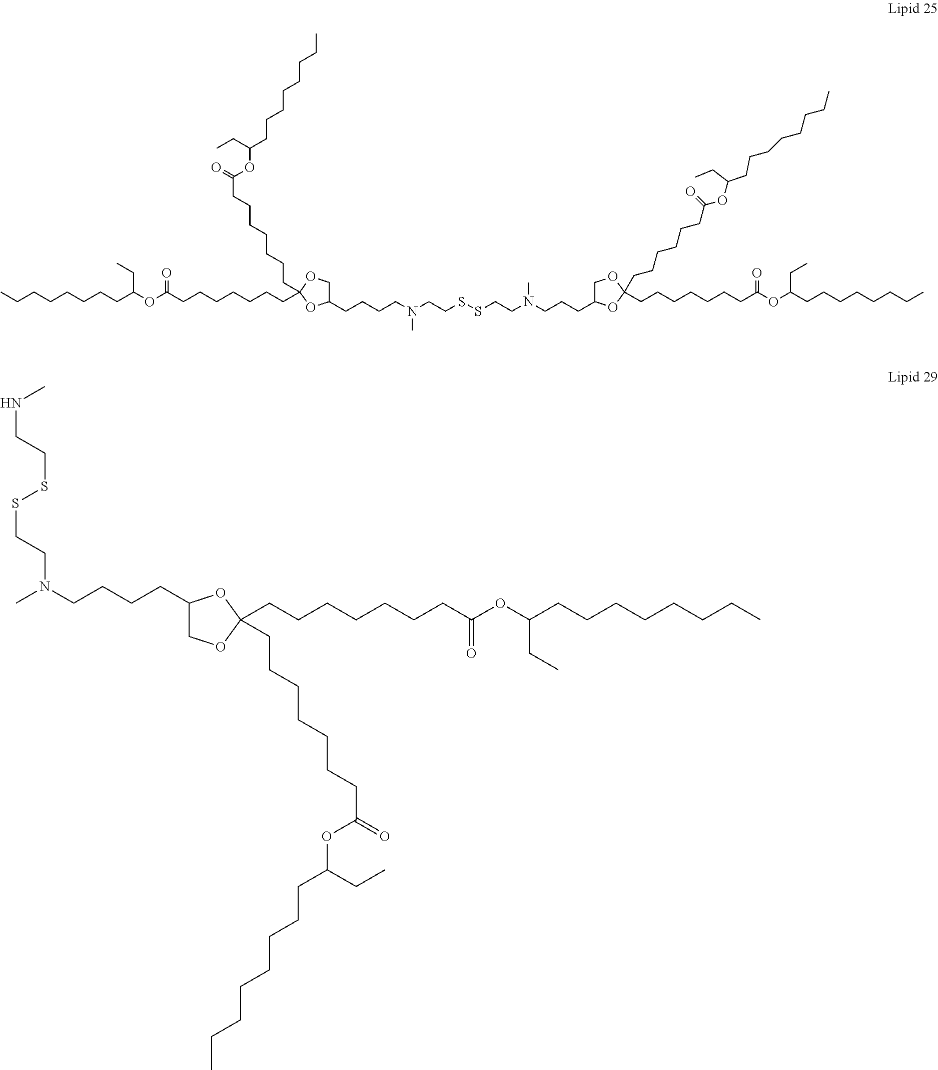

A scheme for the synthesis of Lipid 25 and Lipid 29 is provided in Scheme 23 below.

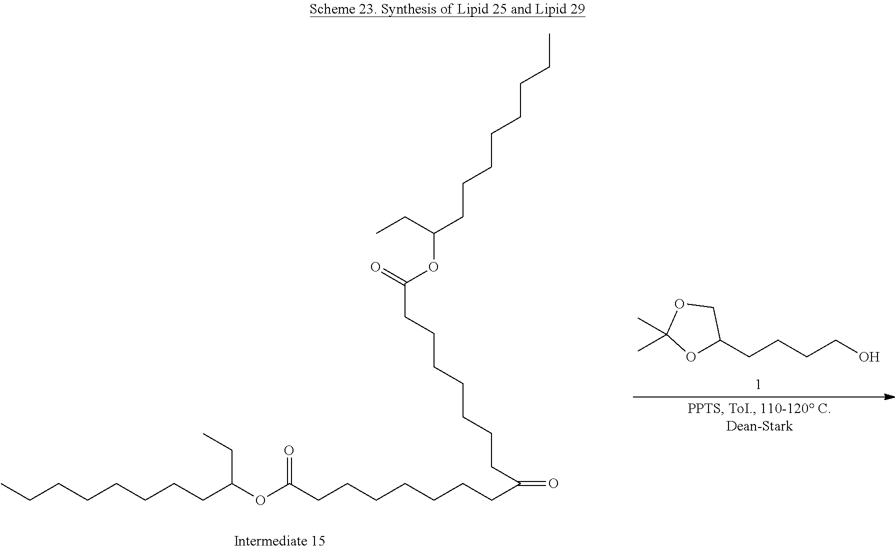 [SMILES: CCCCCCCCC(CC)OC(=O)CCCCCCCC(=O)CCCCCCCC(=O)OC(CC)CCCCCCCC ; CC1(C)OCC(CCCCO)O1]

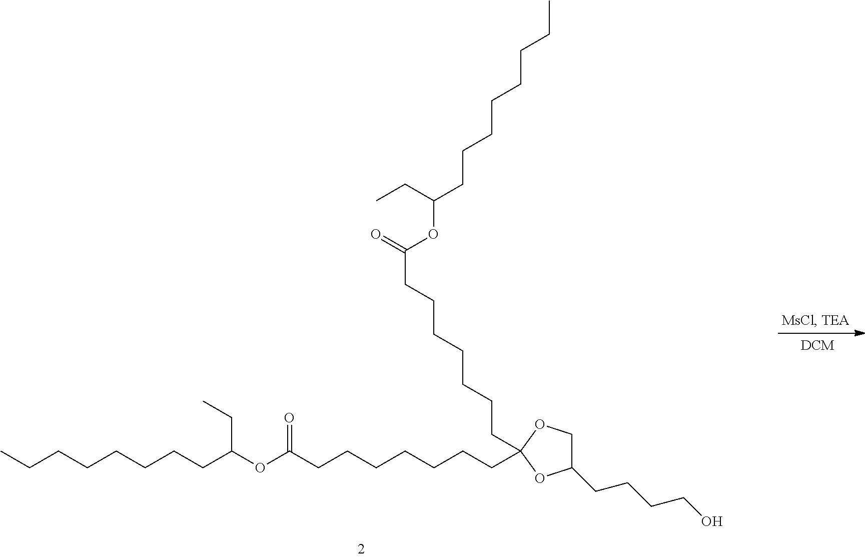 [SMILES: CCCCCCCCC(CC)OC(=O)CCCCCCCC1(CCCCCCCC(=O)OC(CC)CCCCCCCC)OCC(CCCCO)O1]

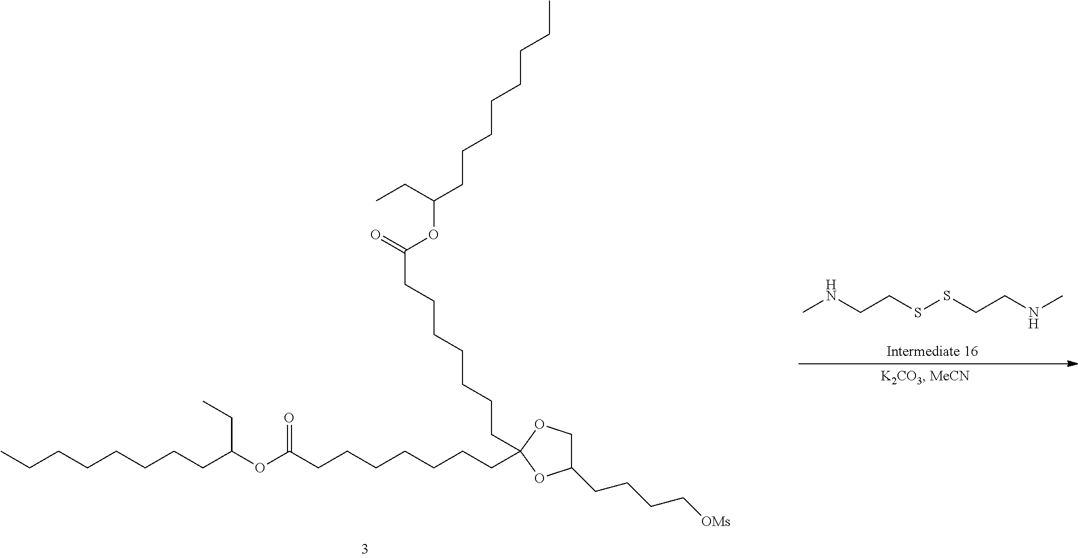

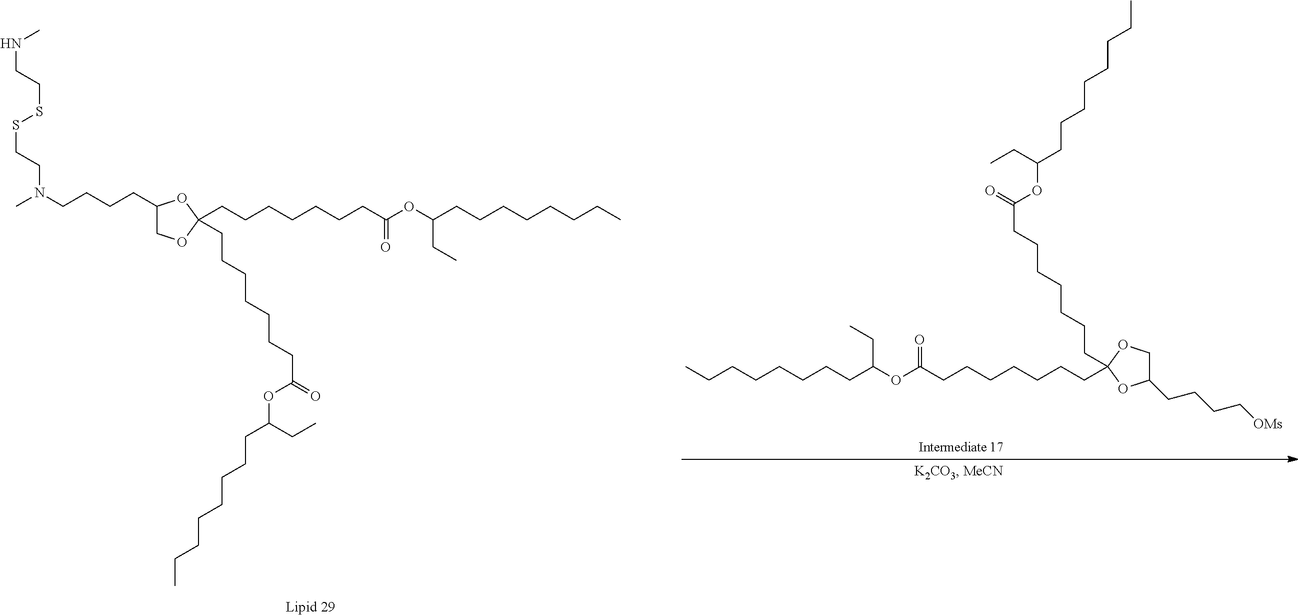 [SMILES: CCCCCCCCC(CC)OC(=O)CCCCCCCC1(CCCCCCCC(=O)OC(CC)CCCCCCCC)OCC(CCCCN(C)CCSSCCNC)O1 ; CCCCCCCCC(CC)OC(=O)CCCCCCCC1(CCCCCCCC(=O)OC(CC)CCCCCCCC)OCC(CCCCOS(C)(=O)=O)O1]

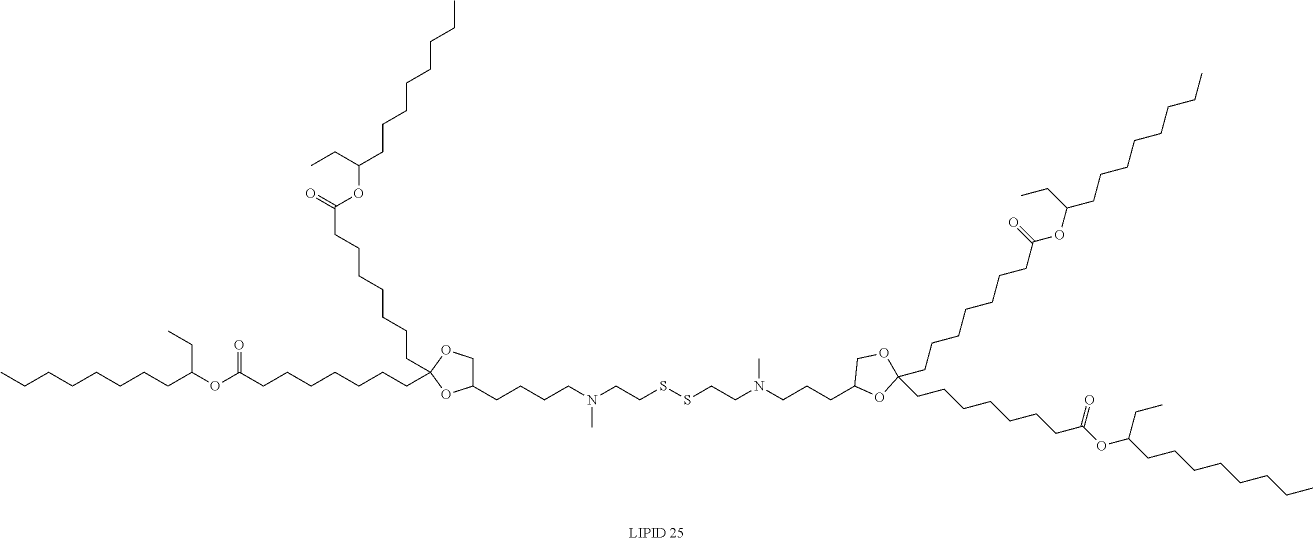 [SMILES: CCCCCCCCC(CC)OC(=O)CCCCCCCC1(CCCCCCCC(=O)OC(CC)CCCCCCCC)OCC(CCCCN(C)CCSSCCN(C)CCCC2COC(CCCCCCCC(=O)OC(CC)CCCCCCCC)(CCCCCCCC(=O)OC(CC)CCCCCCCC)O2)O1]

### Procedure for Preparation of Compound 2

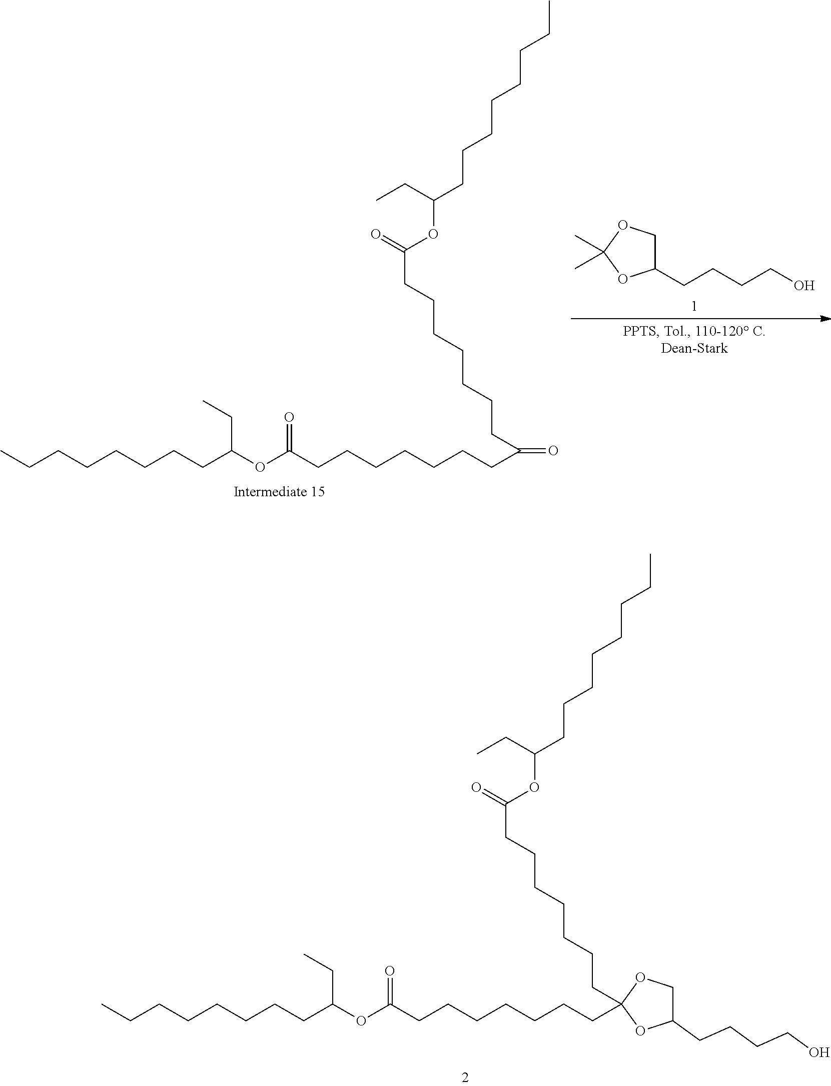 [SMILES: CCCCCCCCC(CC)OC(=O)CCCCCCCC(=O)CCCCCCCC(=O)OC(CC)CCCCCCCC ; CC1(C)OCC(CCCCO)O1 ; CCCCCCCCC(CC)OC(=O)CCCCCCCC1(CCCCCCCC(=O)OC(CC)CCCCCCCC)OCC(CCCCO)O1]

To a mixture of intermediate 15 (2.5 g, 4.01 mmol, 1 eq.) and compound 1 (1.75 g, 10.03 mmol, 2.5 eq.) in toluene (50 mL) was added PPTS (303 mg, 1.21 mmol, 0.3 eq.) at 15-25° C. The mixture was stirred at 110-120° C. for 12 hours equipped with a Dean-Stark. The reaction mixture was cooled to 15-25° C. and poured into saturated NaHCO₃ aqueous solution (50 mL). The mixture was extracted with EtOAc (30 mL*2). The combined organic layers were dried over anhydrous Na₂SO₄, filtered and concentrated under reduced pressure. The residue was purified by column chromatography (SiO₂, Petroleum ether: Ethyl acetate=30:1 to 10:1) to give compound 2 (2.42 g, 79.21% yield, 97.09% purity) as a colorless oil.

¹H NMR (400 MHz, CHLOROFORM-d) δ 4.82 (quin, J=6.0 Hz, 2H), 3.99-4.12 (m, 2H), 3.67 (t, J=6.4 Hz, 2H), 3.40-3.53 (m, 1H), 2.29 (t, J=7.6 Hz, 4H), 1.50-1.65 (m, 22H), 1.21-1.37 (m, 40H), 0.82-0.94 (m, 12H).

### Procedure for Preparation of Compound 3

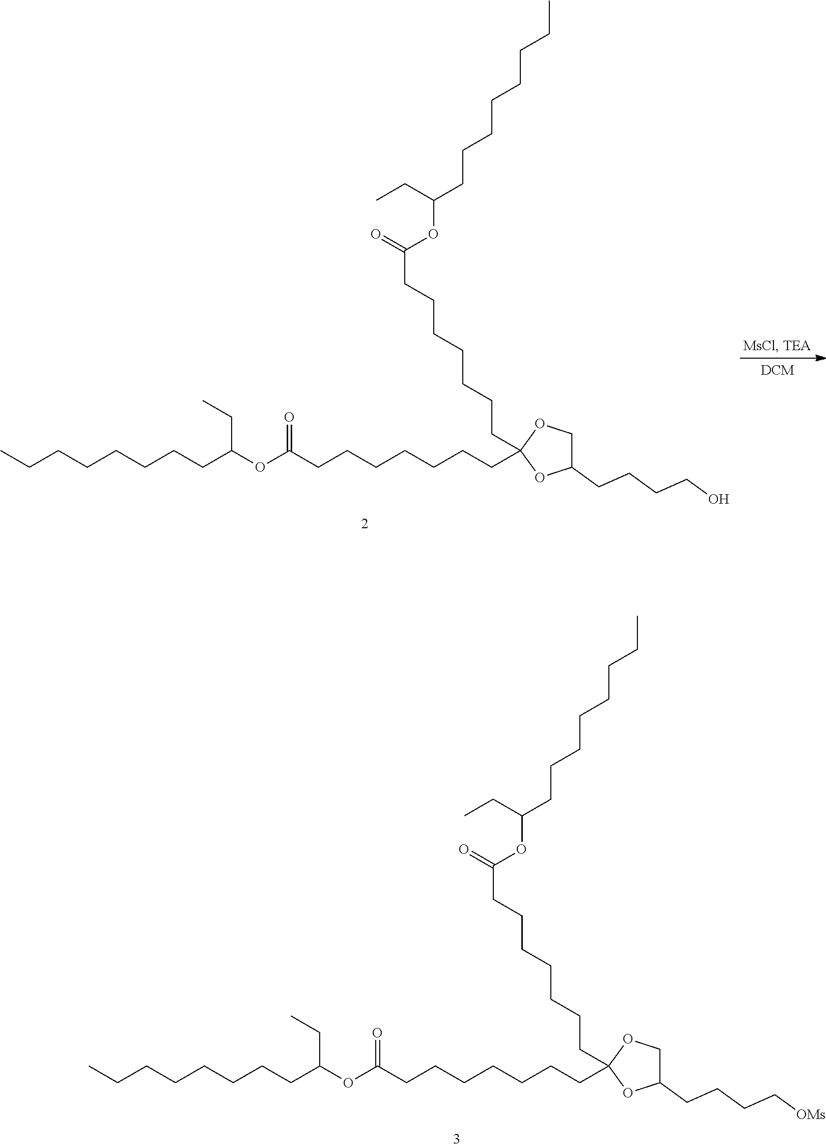 [SMILES: CCCCCCCCC(CC)OC(=O)CCCCCCCC1(CCCCCCCC(=O)OC(CC)CCCCCCCC)OCC(CCCCO)O1 ; CCCCCCCCC(CC)OC(=O)CCCCCCCC1(CCCCCCCC(=O)OC(CC)CCCCCCCC)OCC(CCCCOS(C)(=O)=O)O1]

To a solution of compound 2 (1.5 g, 2.03 mmol, 1 eq.) in DCM (20 mL) was added TEA (1.03 g, 10.15 mmol, 1.41 mL, 5 eq.) and MsCl (697.39 mg, 6.09 mmol, 471.21 μL, 3 eq.) dropwise at 0° C. The mixture was stirred at 25° C. for 2 hours. The reaction mixture was concentrated under reduced pressure. The residue was purified by column chromatography (SiO₂, Petroleum ether/Ethyl acetate=10/1 to 5/1) to give compound 3 (1.57 g, 94.67% yield) as a colorless oil.

¹H NMR (400 MHz, CHLOROFORM-d) δ 4.83 (t, J=6.0 Hz, 2H), 4.26 (t, J=6.4 Hz, 2H), 4.15-3.98 (m, 2H), 3.54-3.40 (m, 1H), 3.03 (s, 3H), 2.30 (t, J=7.6 Hz, 4H), 1.82 (t, J=6.8 Hz, 2H), 1.70-1.61 (m, 6H), 1.59-1.49 (m, 12H), 1.40-1.20 (m, 40H), 0.95-0.84 (m, 12H).

### Procedure for Preparation of Lipid 29

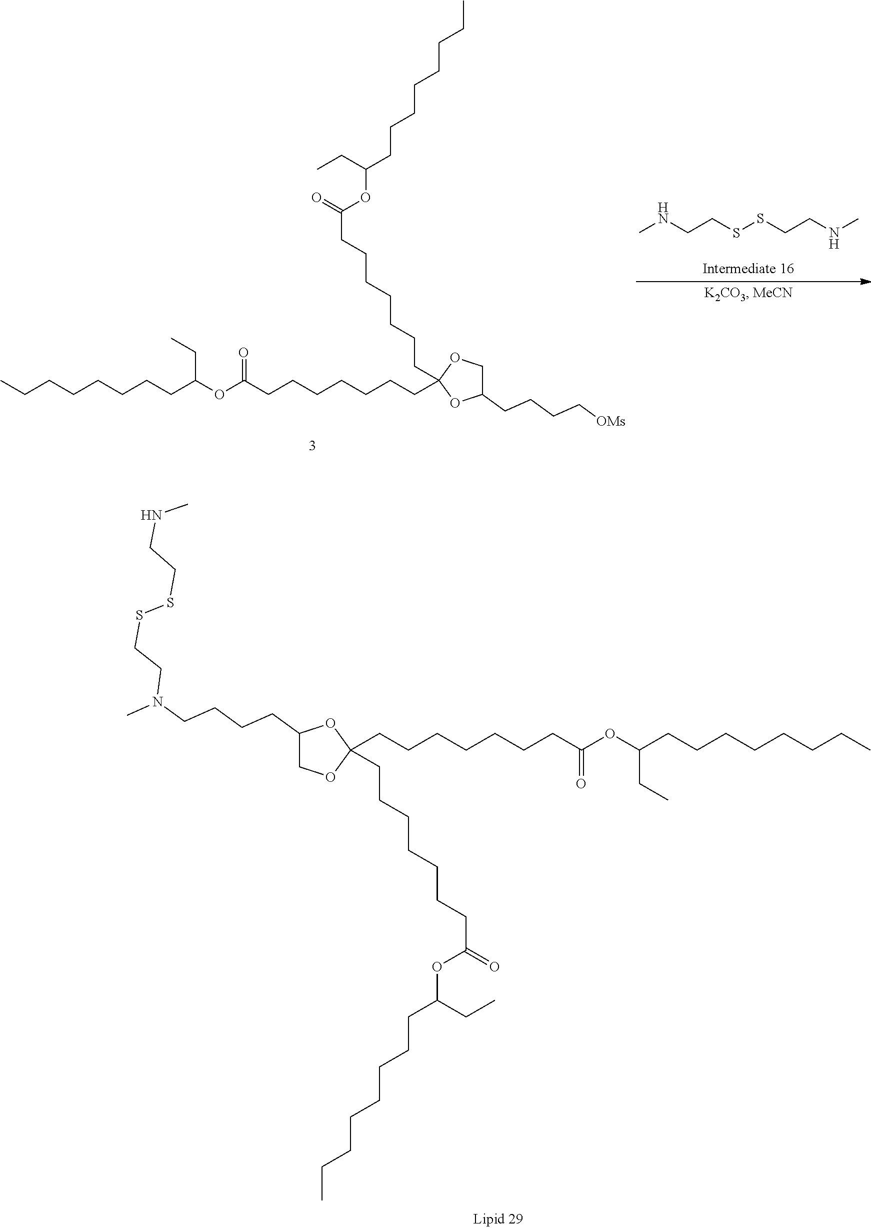

To a solution of compound 3 (600 mg, 734.17 μmol, 1 eq.) and intermediate 16 (198.59 mg, 1.10 mmol, 1.5 eq.) in MeCN (10 mL) was added K₂CO₃ (304.40 mg, 2.20 mmol, 3 eq.) at 25° C. The mixture was stirred at 80° C. for 12 hours. The reaction mixture was filtered and the filtrate was concentrated under reduced pressure. The residue was purified by column chromatography (SiO₂, A/B=100/1 to 5/1, phase A: PE:DCM=3:1, phase B: MeOH, with 0.1% TEA as modifier), followed by prep-HPLC (column: PHS-Phenyl-Hexyl 150*25 mm*7 μm, mobile phase: [water (TFA)-ACN]; gradient: 45%-75% B over 15 min). The combined eluents were immediately neutralized by TEA and concentrated under reduced pressure to remove CAN. The aqueous layer was extracted with a mixed solvent of (PE:EtOAc=3:1.50 mL). The organic phase was washed with water (10 mL*3), dried over anhydrous Na₂SO₄, filtered and concentrated under reduced pressure to give Lipid 29 (120 mg, 18.13% yield) as a yellow oil.

LCMS m/z 901.8 [M+1]⁺. ¹H NMR (400 MHz, CHLOROFORM-d) δ 4.83 (t, J=6.0 Hz, 2H), 4.13-3.98 (m, 2H), 3.45 (s, 1H), 3.27 (br s, 4H), 3.22-3.07 (m, 4H), 2.88 (s, 3H), 2.77 (s, 3H), 2.30 (t, J=7.6 Hz, 4H), 1.88-1.78 (m, 2H), 1.66-1.50 (m, 20H), 1.39-1.24 (m, 42H), 1.00-0.80 (m, 12H).

### Procedure for Preparation of Lipid 25

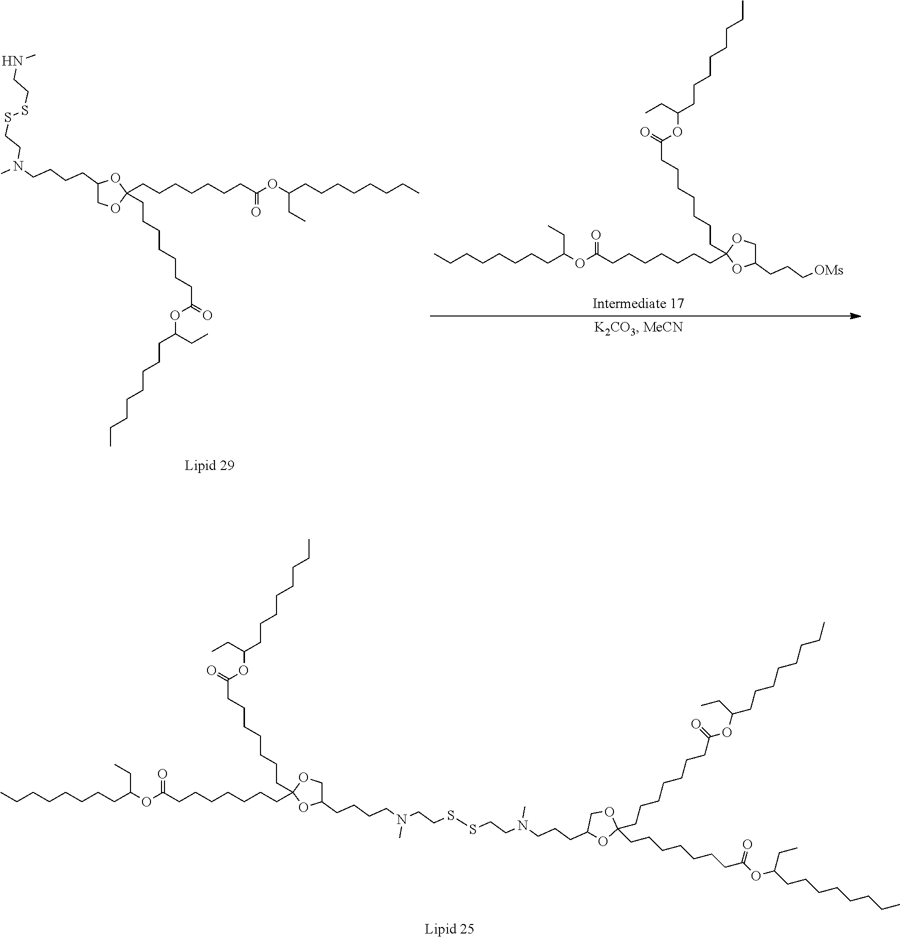 [SMILES: CCCCCCCCC(CC)OC(=O)CCCCCCCC1(CCCCCCCC(=O)OC(CC)CCCCCCCC)OCC(CCCCN(C)CCSSCCNC)O1 ; CCCCCCCCC(CC)OC(=O)CCCCCCCC1(CCCCCCCC(=O)OC(CC)CCCCCCCC)OCC(CCCOS(C)(=O)=O)O1 ; CCCCCCCCC(CC)OC(=O)CCCCCCCC1(CCCCCCCC(=O)OC(CC)CCCCCCCC)OCC(CCCCN(C)CCSSCCN(C)CCCC2COC(CCCCCCCC(=O)OC(CC)CCCCCCCC)(CCCCCCCC(=O)OC(CC)CCCCCCCC)O2)O1]

To a mixture of Lipid 29 (250 mg, 281.71 mol, 1 eq.) and intermediate 17 (345.34 mg, 422.56 μmol, 1.5 eq.) in MeCN (5 mL) was added K₂CO₃ (116.80 mg, 845.12 μmol, 3 eq.) at 25° C. The mixture was stirred at 100° C. for 12 hours. The reaction mixture was filtered and the filtrate was concentrated under reduced pressure. The residue was purified by column chromatography (SiO₂, PE/EA=10/1 to 5/1, with 0.1% TEA as modifier), followed by column chromatography (SiO₂, A/B=100/1 to 5/1, phase A: PE:DCM=3:1, phase B: EtOAc, with 0.1% TEA as modifier) multiple times to give Lipid 25 (80 mg, 38.04% yield, 95.02% purity) as a yellow oil.

LCMS m/z 805.0 [M/2+1]⁺. ¹H NMR (400 MHz, CHLOROFORM-d) δ 4.85 (quin, J=6.0 Hz, 4H), 4.13-4.04 (m, 4H), 3.53-3.45 (m, 2H), 2.90-2.79 (m, 4H), 2.78-2.66 (m, 4H), 2.50-2.39 (m, 4H), 2.37-2.22 (m, 14H), 1.61-1.51 (m, 42H), 1.45-1.21 (m, 80H), 0.99-0.86 (m, 24H).

### Synthesis of Lipid 26

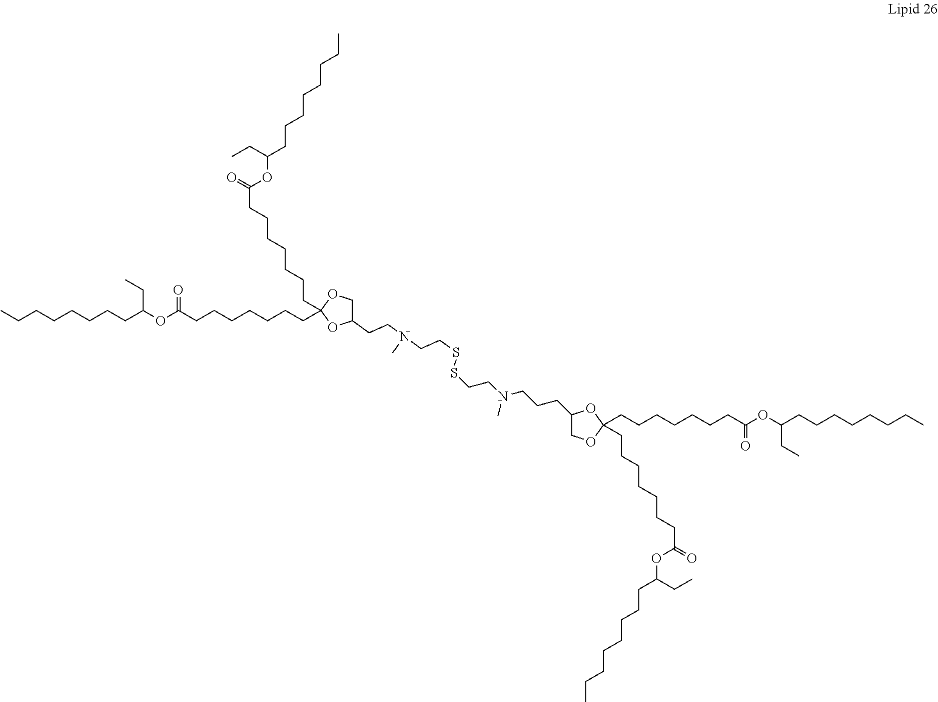 [SMILES: CCCCCCCCC(CC)OC(=O)CCCCCCCC1(CCCCCCCC(=O)OC(CC)CCCCCCCC)OCC(CCCN(C)CCSSCCN(C)CCC2COC(CCCCCCCC(=O)OC(CC)CCCCCCCC)(CCCCCCCC(=O)OC(CC)CCCCCCCC)O2)O1]

A scheme for the synthesis of Lipid 26 is provided in Scheme 24 below.

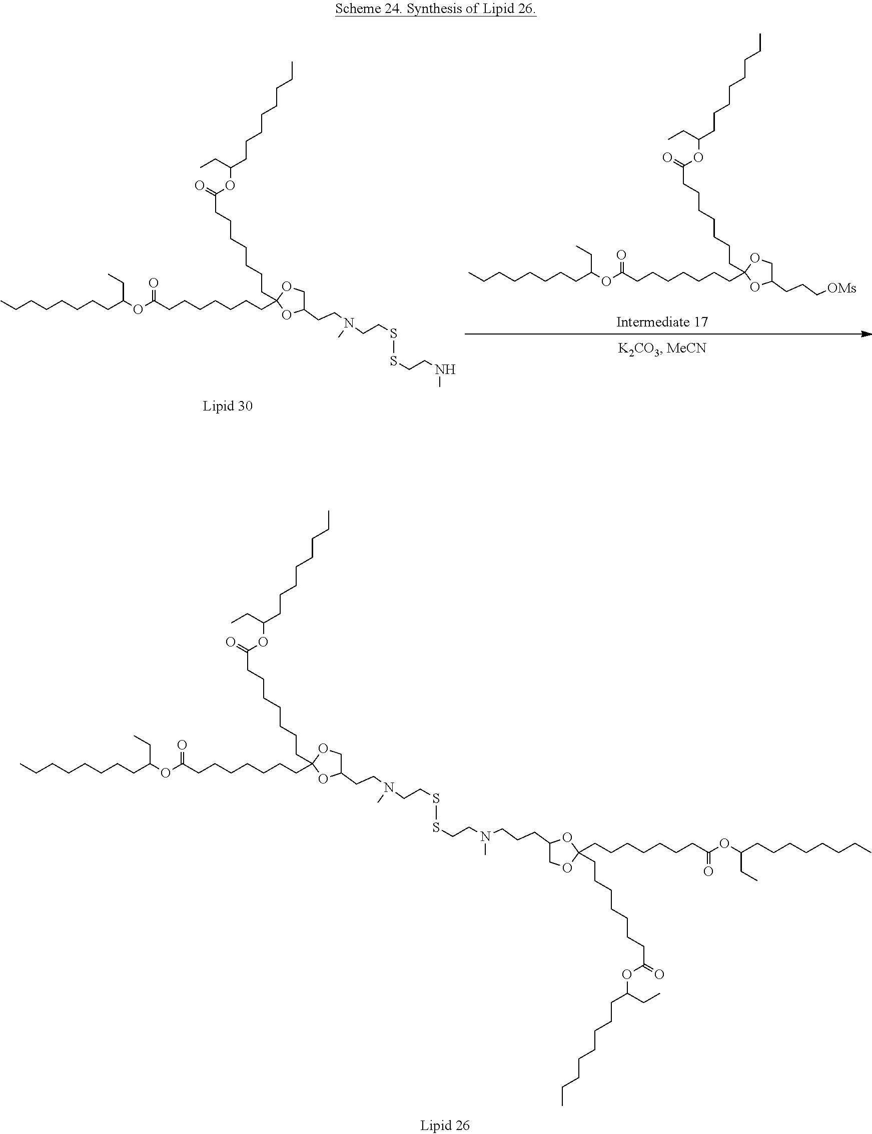 [SMILES: CCCCCCCCC(CC)OC(=O)CCCCCCCC1(CCCCCCCC(=O)OC(CC)CCCCCCCC)OCC(CCN(C)CCSSCCNC)O1 ; CCCCCCCCC(CC)OC(=O)CCCCCCCC1(CCCCCCCC(=O)OC(CC)CCCCCCCC)OCC(CCCOS(C)(=O)=O)O1 ; CCCCCCCCC(CC)OC(=O)CCCCCCCC1(CCCCCCCC(=O)OC(CC)CCCCCCCC)OCC(CCCN(C)CCSSCCN(C)CCC2COC(CCCCCCCC(=O)OC(CC)CCCCCCCC)(CCCCCCCC(=O)OC(CC)CCCCCCCC)O2)O1]

### Procedure for Preparation of Lipid 26

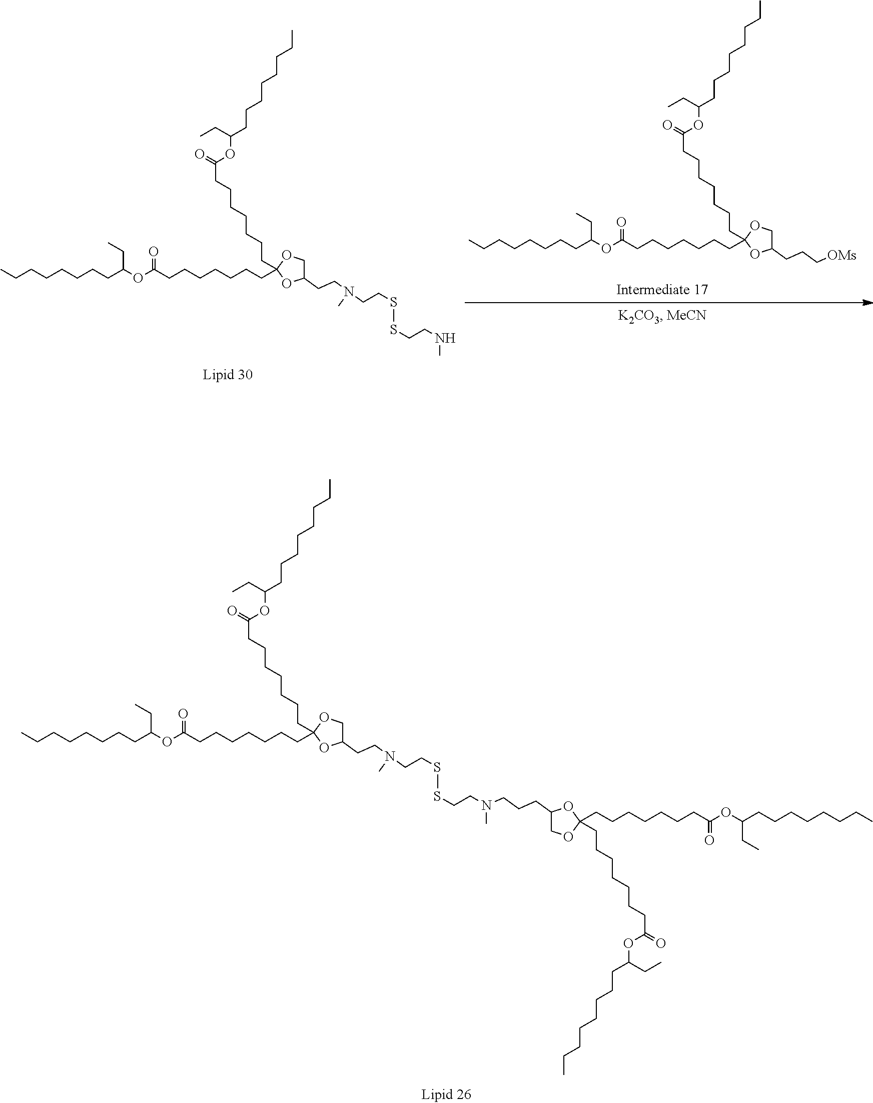 [SMILES: CCCCCCCCC(CC)OC(=O)CCCCCCCC1(CCCCCCCC(=O)OC(CC)CCCCCCCC)OCC(CCN(C)CCSSCCNC)O1 ; CCCCCCCCC(CC)OC(=O)CCCCCCCC1(CCCCCCCC(=O)OC(CC)CCCCCCCC)OCC(CCCOS(C)(=O)=O)O1 ; CCCCCCCCC(CC)OC(=O)CCCCCCCC1(CCCCCCCC(=O)OC(CC)CCCCCCCC)OCC(CCCN(C)CCSSCCN(C)CCC2COC(CCCCCCCC(=O)OC(CC)CCCCCCCC)(CCCCCCCC(=O)OC(CC)CCCCCCCC)O2)O1]

To a mixture of Lipid 30 (200 mg, 228.98 μmol, 1 eq.) and intermediate 17 (200.00 mg, 249.00 μmol, 1.09 eq.) in MeCN (5 mL) was added K₂CO₃ (94.94 mg, 686.95 μmol, 3 eq.). The mixture was stirred at 80° C. for 16 hours. The reaction mixture was poured into ice-water (15 mL), and then extracted with DCM (20 mL*3). The combined organic layers were washed with brine (20 mL), dried over anhydrous Na₂SO₄, filtered and concentrated under reduced pressure. The residue was purified by column chromatography (SiO₂, A/B=I/O to 10/1, phase A: PE/EA=3/2, phase B: Acetone, with 0.1% TEA as modifier), followed by prep-TLC (SiO₂, DCM:MeOH=20:1), and then further purified by prep-NPLC (Welch Ultimate XB—SiOH 250*50*10 μm; mobile phase: [Hexane-EtOH]; B %: 1%, isocratic elution mode) to give Lipid 26 (24 mg, 6.63% yield, 95.22% purity) as a yellow oil.

LCMS m/z 1580.4 [M+1]⁺. ¹H NMR (400 MHz, CHLOROFORM-d) δ 4.82 (quin, J=6.4 Hz, 4H), 4.12-4.01 (m, 4H), 3.50-3.42 (m, 2H), 2.85-2.76 (m, 4H), 2.69 (br d, J=7.2 Hz, 4H), 2.57-2.37 (m, 4H), 2.32-2.22 (m, 13H), 1.64-1.49 (m, 44H), 1.39-1.22 (m, 82H), 0.91-0.85 (m, 24H).

### Synthesis of Lipid 27 and Lipid 30

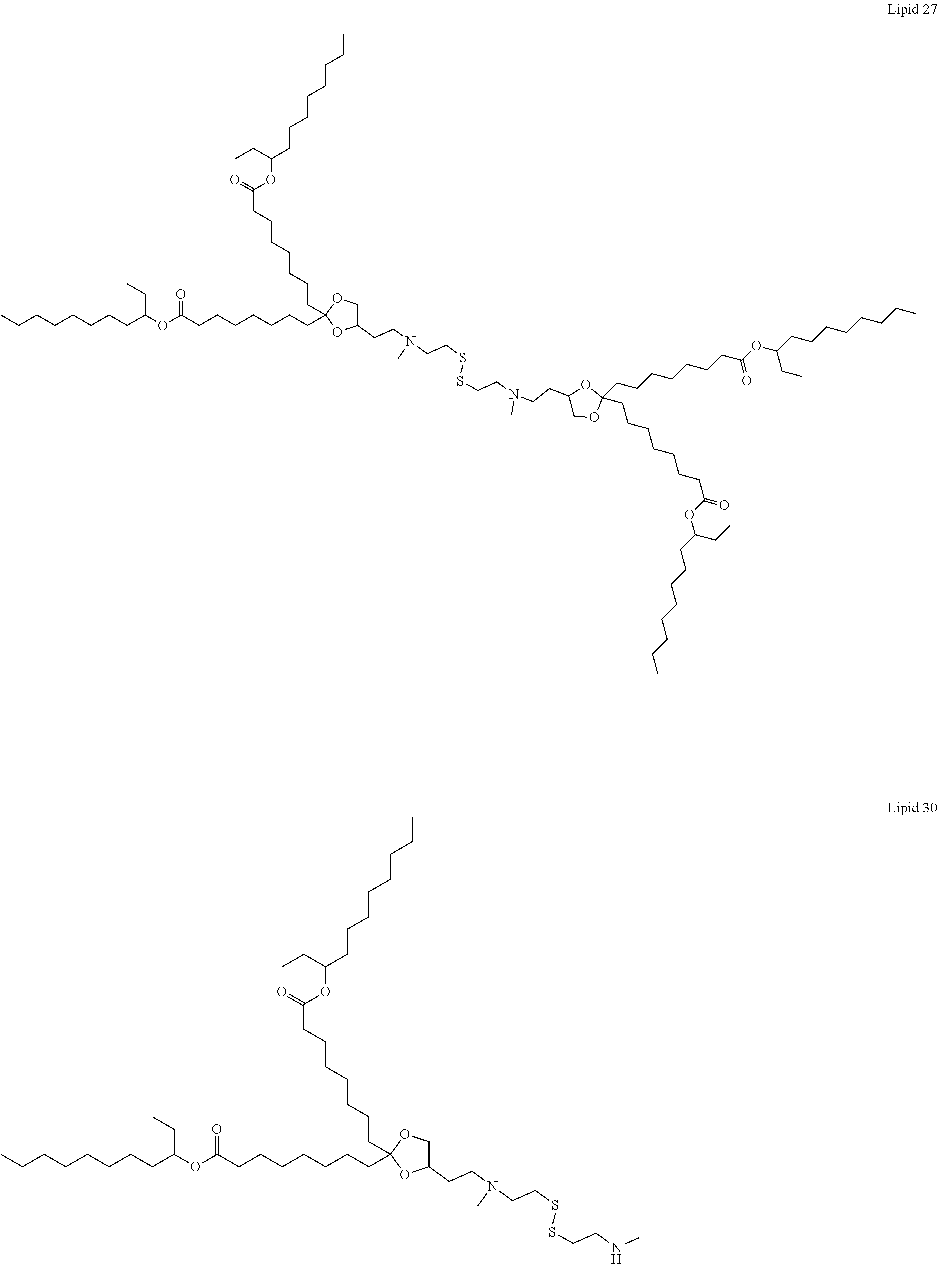

A scheme for the synthesis of Lipid 27 and Lipid 30 is provided in Scheme 25 below.

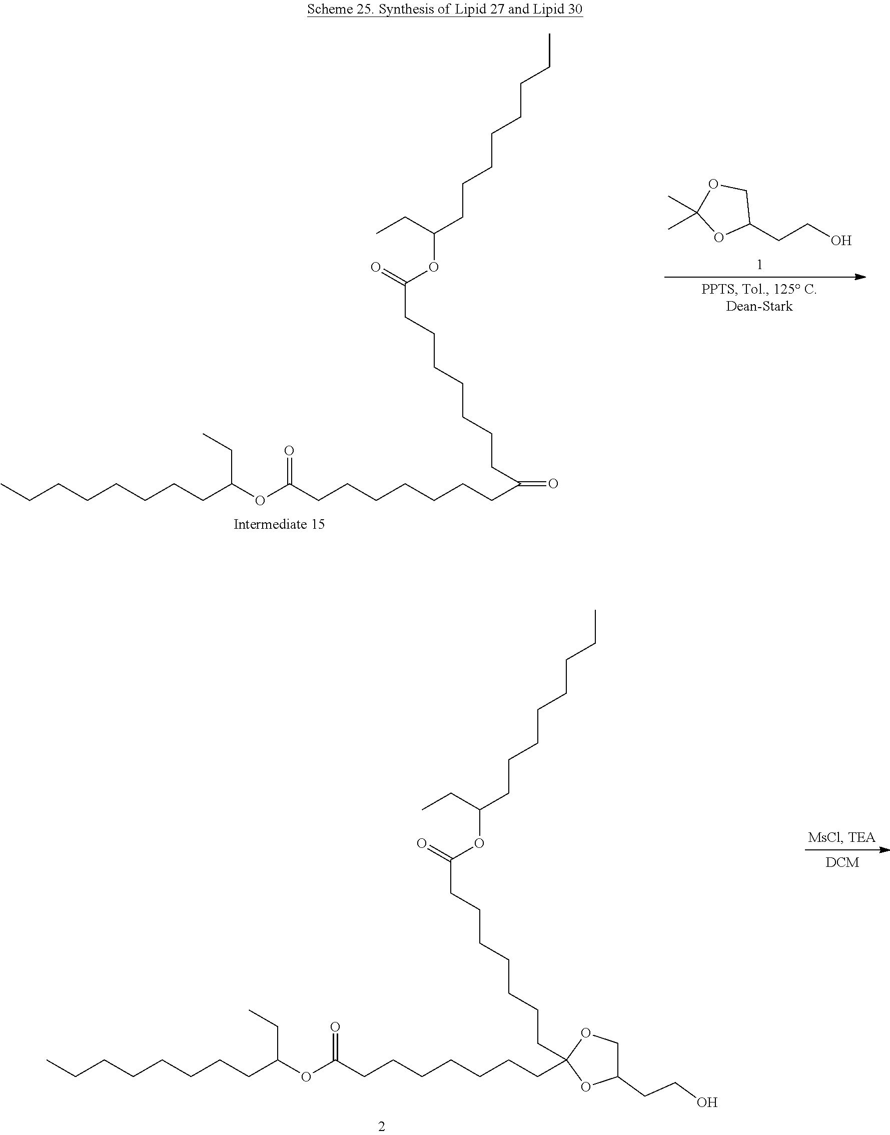

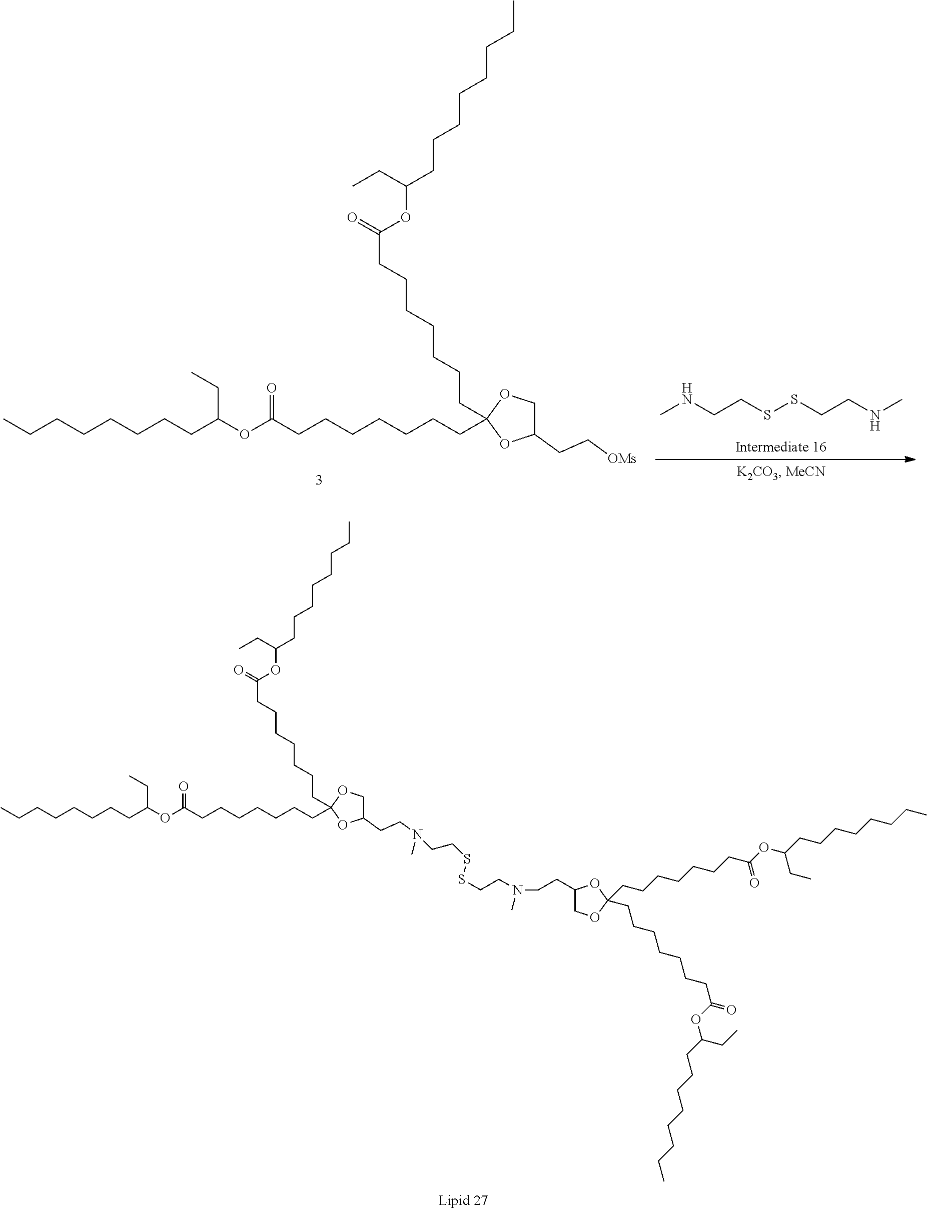

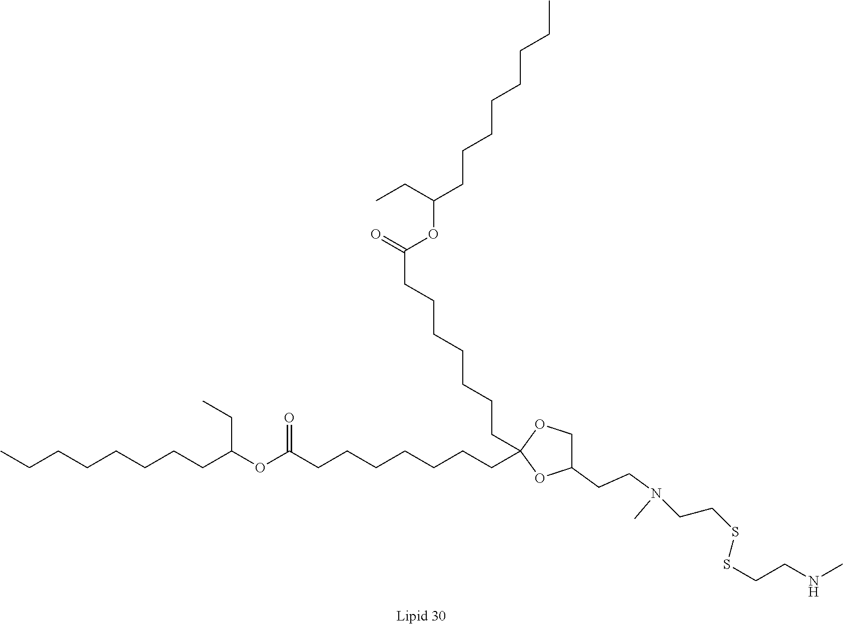 [SMILES: CCCCCCCCC(CC)OC(=O)CCCCCCCC1(CCCCCCCC(=O)OC(CC)CCCCCCCC)OCC(CCN(C)CCSSCCNC)O1]

### Procedure for Preparation of Compound 2

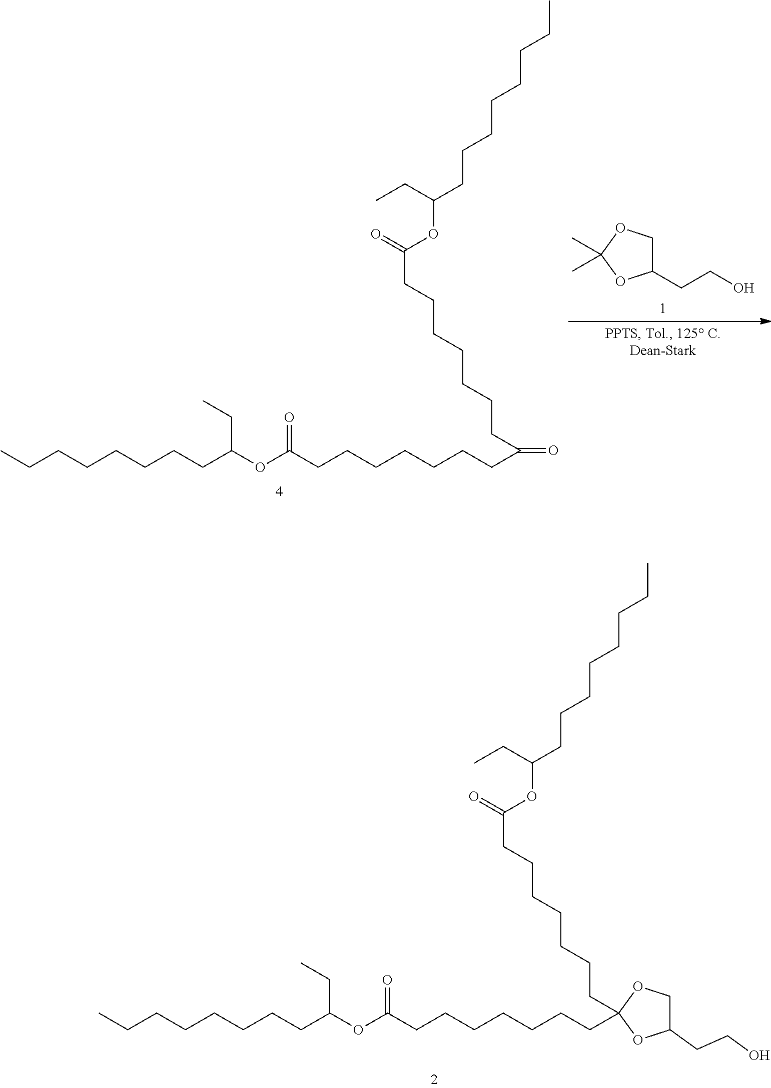

To a mixture of intermediate 15 (2 g, 3.21 mmol, 1 eq.) and compound 1 (1.17 g, 8.03 mmol, 1.14 mL, 2.5 eq.) in toluene (30 mL) was added PPTS (242.02 mg, 963.08 μmol, 0.3 eg.) at 25° C. The mixture was stirred at 125° C. for 12 hours equipped with a Dean-Stark. The reaction mixture was poured into saturated NaHCO₃ aqueous solution (40 mL) and stirred for 2 mins. The aqueous phase was extracted with DCM (30 mL*3). The combined organic phases were washed with brine (40 mL), dried over anhydrous Na₂SO₄, filtered and concentrated under vacuum. The residue was purified by column chromatography (SiO₂, Petroleum ether/Ethyl acetate=10/1 to 4/1) to give compound 2 (1.3 g, 56.95% yield) as a colorless oil.

¹H NMR (400 MHz, CHLOROFORM-d) δ 4.85 (quin, J=6.4 Hz, 2H), 4.33-4.21 (m, 1H), 4.18-4.07 (m, 1H), 3.84 (br t, J=4.8 Hz, 2H), 3.56 (t, J=8.0 Hz, 1H), 2.32 (t, J=7.6 Hz, 4H), 1.90-1.81 (m, 2H), 1.65-1.47 (m, 16H), 1.43-1.22 (m, 40H), 0.97-0.87 (m, 12H).

### Procedure for Preparation of Compound 3

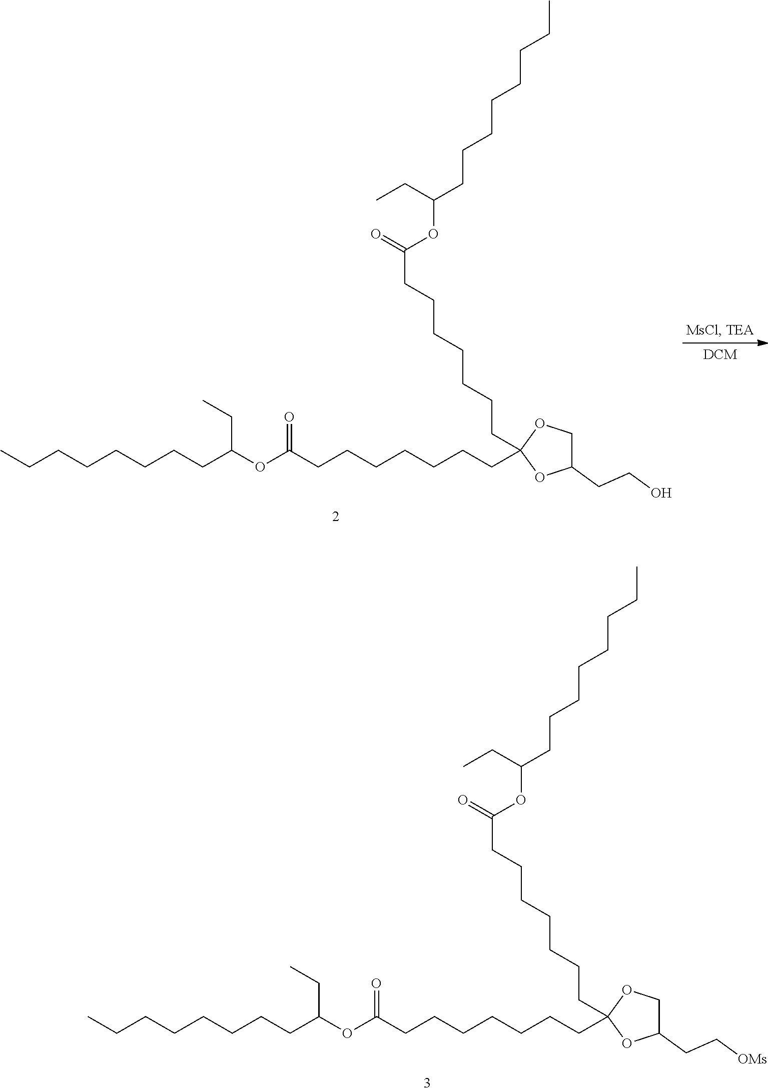 [SMILES: CCCCCCCCC(CC)OC(=O)CCCCCCCC1(CCCCCCCC(=O)OC(CC)CCCCCCCC)OCC(CCO)O1 ; CCCCCCCCC(CC)OC(=O)CCCCCCCC1(CCCCCCCC(=O)OC(CC)CCCCCCCC)OCC(CCOS(C)(=O)=O)O1]

To a mixture of compound 2 (1.3 g, 1.83 mmol, 1 eq.) and TEA (924.94 mg, 9.14 mmol, 1.27 mL, 5 eq.) in DCM (20 mL) was added MsCl (628.25 mg, 5.48 mmol, 424.49 μL, 3 eq.) dropwise at 0° C. The mixture was stirred at 25° C. for 2 hours. The reaction mixture was concentrated under reduced pressure. The residue was purified by column chromatography (SiO₂, Petroleum ether/Ethyl acetate=10/1 to 5/1) to give compound 3 (1.3 g, 90.11% yield) as a colorless oil.

¹H NMR (400 MHz, CHLOROFORM-d) δ 4.83 (t, J=6.0 Hz, 2H), 4.51-4.30 (m, 2H), 4.27-4.05 (m, 2H), 3.54 (t, J=7.6 Hz, 1H), 3.05 (s, 3H), 2.31 (t, J=7.5 Hz, 4H), 2.09-1.91 (m, 2H), 1.69-1.60 (m, 6H), 1.58-1.45 (m, 10H), 1.41-1.22 (m, 40H), 0.96-0.84 (m, 12H).

### Procedure for Preparation of Lipid 27 and Lipid 30

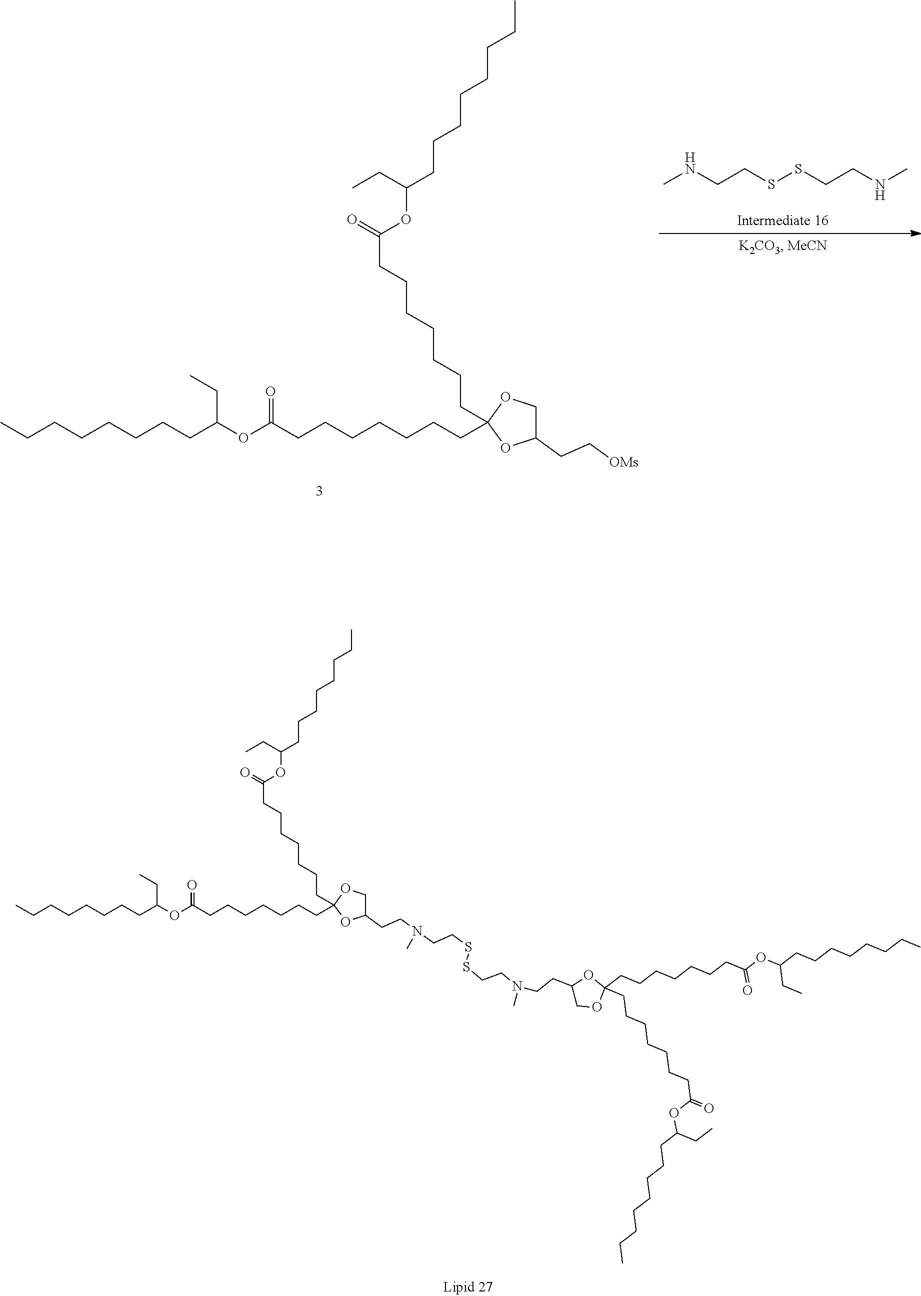

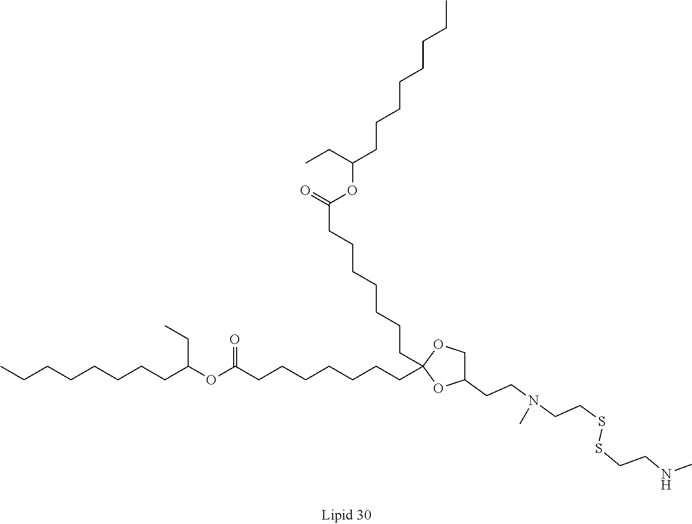 [SMILES: CCCCCCCCC(CC)OC(=O)CCCCCCCC1(CCCCCCCC(=O)OC(CC)CCCCCCCC)OCC(CCN(C)CCSSCCNC)O1]

To a suspension of compound 3 (1.575 g, 2.00 mmol, 1 eq.) and K₂CO₃ (827.45 mg, 5.99 mmol, 3 eq.) in MeCN (16 mL) was added intermediate 16 (707.59 mg, 2.79 mmol, 1.4 eq, 2HCl salt) at 25° C., and then the mixture was stirred at 80° C. for 12 hours. The reaction mixture was filtered and the filtrate was concentrated under reduced pressure. The residue was purified by column chromatography (SiO₂, PE/EA=10/1 to 5/1, with 0.1% TEA as modifier) to give crude product 1 (Lipid 27), followed by eluting with (A/B=100/1 to 5/1, phase A: PE:DCM=3:1, phase B: MeOH, with 0.1% TEA as modifier) to give crude product 2 (Lipid 30).

The crude product 1 was purified by prep-TLC (SiO₂, DCM:MeOH=15:1), followed by prep-NPLC (column: Welch Ultimate XB—SiOH 250*50*10 μm; mobile phase: [Hexane-EtOH]; B %: 1%, isocratic elution mode) to give Lipid 27 (60 mg, 73.50% yield, 98.58% purity) as a yellow oil.

LCMS m/z 1566.5 [M+1]⁺. ¹H NMR (400 MHz, CHLOROFORM-d) δ 4.83 (t, J=6.4 Hz, 4H), 4.10 (br s, 4H), 3.61-3.43 (m, 2H), 2.95-2.75 (m, 4H), 2.74-2.65 (m, 2H), 2.63-2.40 (m, 4H), 2.38-2.19 (m, 12H), 1.73-1.48 (m, 40H), 1.44-1.21 (m, 80H), 1.01-0.80 (m, 24H).

The crude product 2 was purified by prep-HPLC (column: PHS-Phenyl-Hexyl 150*25 mm*7 μm; mobile phase: [water (TFA)-ACN]; gradient: 45%-75% B over 15 min). The combined eluents were immediately neutralized by TEA and concentrated under reduced pressure to remove ACN. The aqueous layer was extracted with a mixed solvent of (PE:EtOAc=3:1, 50 mL). The combined organic phases were washed with water (10 mL*3), dried over anhydrous Na₂SO₄, filtered and concentrated under reduced pressure to give Lipid 30 (30 mg, 8.14% yield, 95.15% purity) as a light-yellow oil.

LCMS m/z 873.7 [M+1]⁺. 1H NMR (400 MHz, CHLOROFORM-d) δ 4.81 (quin, J=6.4 Hz, 2H), 4.13-4.03 (m, 2H), 3.51-3.43 (m, 1H), 2.99-2.92 (m, 2H), 2.89-2.79 (m, 4H), 2.73-2.68 (m, 2H), 2.49 (s, 3H), 2.32-2.26 (m, 7H), 1.91-1.43 (m, 20H), 1.38-1.20 (m, 40H), 0.91-0.85 (m, 12H).

### Synthesis of Lipid 31

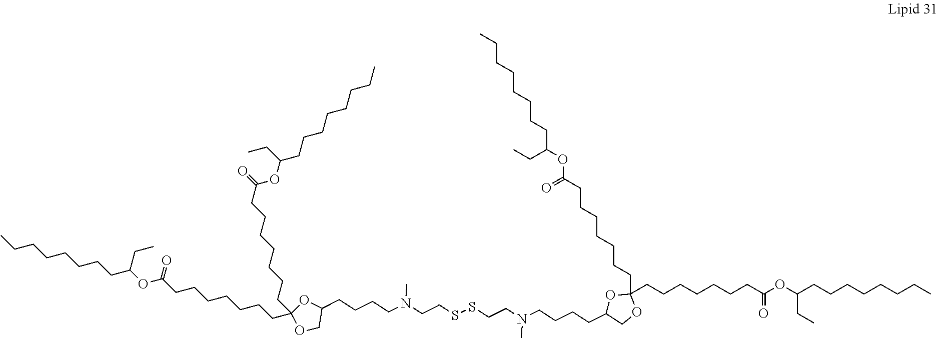 [SMILES: CCCCCCCCC(CC)OC(=O)CCCCCCCC1(CCCCCCCC(=O)OC(CC)CCCCCCCC)OCC(CCCCN(C)CCSSCCN(C)CCCCC2COC(CCCCCCCC(=O)OC(CC)CCCCCCCC)(CCCCCCCC(=O)OC(CC)CCCCCCCC)O2)O1]

A scheme for the synthesis of Lipid 31 is provided in Scheme 26 below.

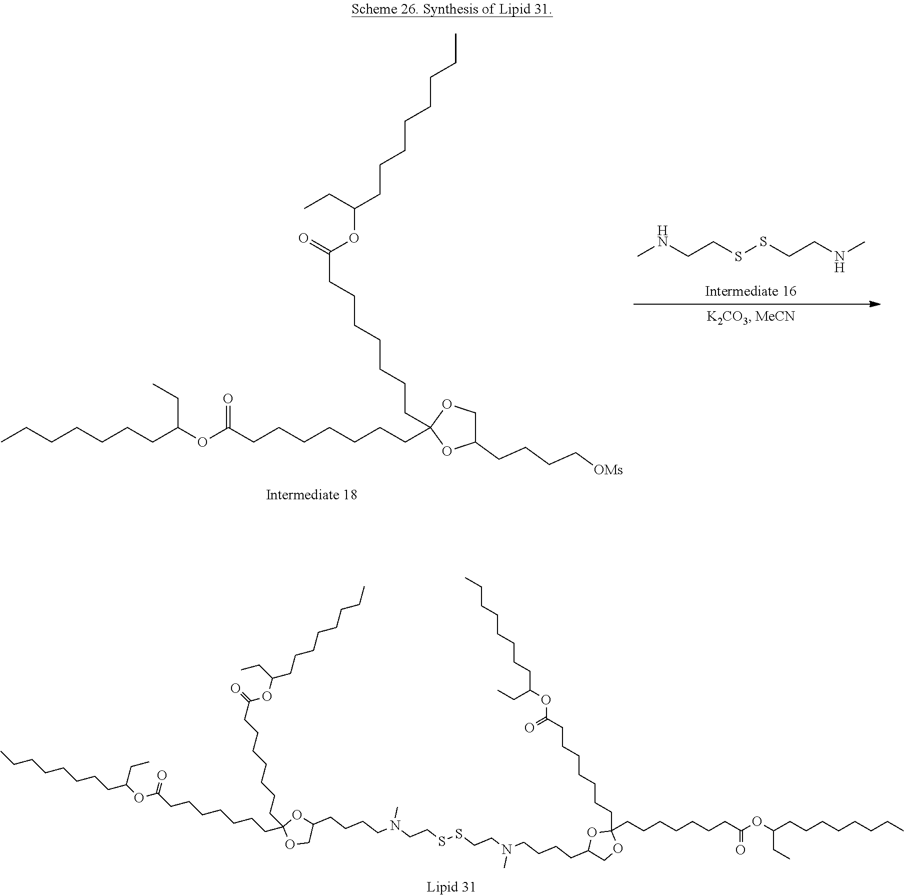

To a mixture of intermediate 16 (100 mg, 394.86 μmol, 1 eq., 2HCl salt) and intermediate 18 (968.09 mg, 1.18 mmol, 3 eq.) in MeCN (3 mL) was added K₂CO₃ (163.71 mg, 1.18 mmol, 3 eq.) at 25° C. The mixture was stirred at 100° C. for 12 hours. The reaction mixture was filtered and the filtrate was concentrated under reduced pressure. The residue was purified by column chromatography (SiO₂, PE/EA=10/1 to 5/1, with 0.1% TEA as modifier), followed by prep-NPLC (column: Welch Ultimate XB—SiOH 250*50*10 μm; mobile phase: [Hexane-EtOH]; B %: 1%, isocratic elution mode) to give Lipid 31 (121 mg, 19.37% yield, 95.47% purity) as a yellow oil.

LCMS m/z 1622.5 [M+1]⁺. ¹H NMR (400 MHz, CHLOROFORM-d) δ 4.83 (t, J=6.0 Hz, 4H), 4.14-3.99 (m, 4H), 3.52-3.41 (m, 2H), 2.91-2.78 (m, 4H), 2.78-2.59 (m, 4H), 2.39 (br d, J=6.0 Hz, 4H), 2.34-2.25 (m, 12H), 1.64-1.48 (m, 46H), 1.41-1.22 (m, 80H), 0.97-0.82 (m, 24H).

### Synthesis of Head D

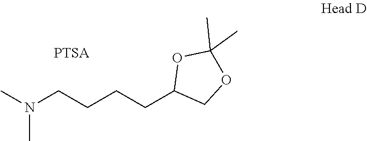

A scheme for the synthesis of Head D is provided in Scheme 27 below.

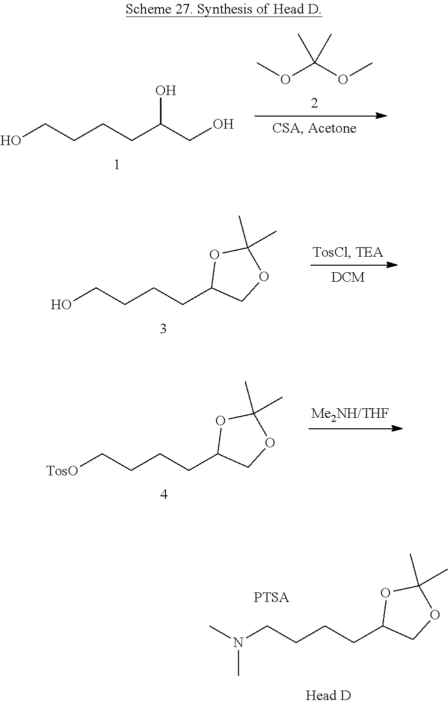

### Procedure for Preparation of Compound 3

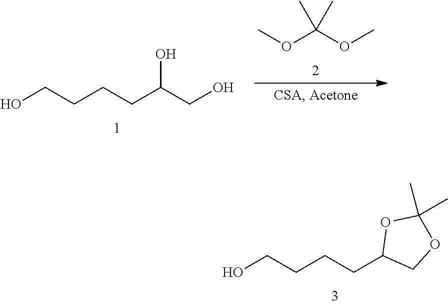

To a solution of compound 1 (10 g, 74.53 mmol, 1 eq.) in acetone (100 mL) was added compound 2 (15.52 g, 149.06 mmol, 18.26 mL, 2 eq.) and CSA (1.73 g, 7.45 mmol, 0.1 eq.). The mixture was stirred at 30° C. for 12 hours. 4A molecular sieve (10 g) was added into the mixture and the resulting suspension was stirred at 30° C. for another 20 mins. The mixture was filtered and the filtrate was concentrated under reduced pressure. The residue was purified by column chromatography (SiO₂, Petroleum ether/Ethyl acetate=10/1˜5/1˜1/3) to give compound 3 (7.2 g, 49.90% yield, >90% purity) as a yellow oil.

¹H NMR (400 MHz, CHLOROFORM-d) δ 4.17-3.99 (m, 2H), 3.67 (t, J=6.4 Hz, 2H), 3.56-3.48 (m, 1H), 1.74-1.48 (m, 6H), 1.41 (s, 3H), 1.36 (s, 3H).

### Procedure for Preparation of Compound 4

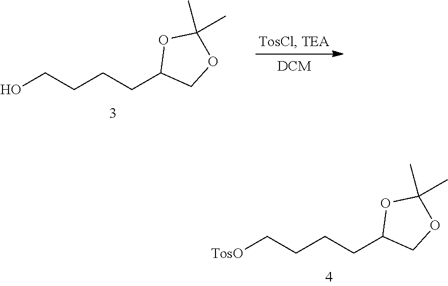

To a mixture of compound 3 (3.1 g, 17.79 mmol, 1 eq.) and TEA (5.40 g, 53.38 mmol, 7.43 mL, 3 eq.) in DCM (40 mL) was added TosCl (3.87 g, 20.28 mmol, 1.14 eq.) in portions at 25° C., and then the resulting mixture was stirred at 25° C. for 12 hours. The reaction mixture was filtered and the filtrate was concentrated under reduced pressure. The residue was purified by column chromatography (SiO₂, Petroleum ether/Ethyl acetate=20/1 to 5/1) to give compound 4 (4.1 g, 63.15% yield, >90% purity) as a yellow oil.

¹H NMR (400 MHz, CHLOROFORM-d) δ 7.81 (d, J=8.4 Hz, 2H), 7.36 (d, J=8.4 Hz, 2H), 4.09-3.97 (m, 4H), 3.54-3.43 (m, 1H), 2.47 (s, 3H), 1.71 (quin, J=6.8 Hz, 2H), 1.64-1.41 (m, 4H), 1.40 (s, 3H), 1.35 (s, 3H).

### Procedure for Preparation of Head D

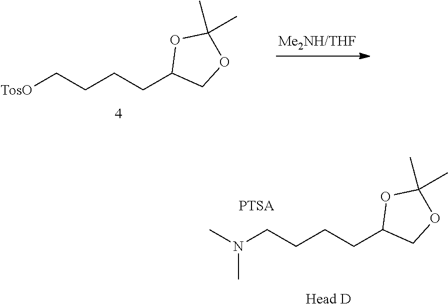

A solution of compound 4 (4.1 g, 12.48 mmol, 1 eq.) in Me₂NH/THF (2 M, 45.56 mL, 7.30 eq.) was stirred at 25° C. for 12 hours. The mixture was concentrated under reduced pressure. The residue was triturated with EA/PE=5/1 (50 mL) to give Head D (3.2 g, crude, PTSA salt) as a pink solid, which was used directly for next step without further purification.

MS m/z: 202.1 [M+1]⁺. ¹H NMR (400 MHz, CHLOROFORM-d) δ 11.01-10.83 (m, 1H), 7.80 (d, J=8.0 Hz, 2H), 7.22 (d, J=8.0 Hz, 2H), 4.11-3.97 (m, 2H), 3.55-3.46 (m, 1H), 3.10-2.99 (m, 2H), 2.91 (d, J=4.8 Hz, 6H), 2.39 (s, 3H), 1.92-1.77 (m, 2H), 1.68-1.44 (m, 4H), 1.42 (s, 3H), 1.37 (s, 3H).

### Synthesis of Lipid 32

### Procedure for Preparation of Compound 2

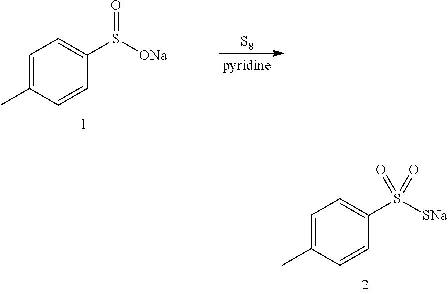

To a solution of compound 1 (10 g, 56.12 mmol, 1 eq.) in pyridine (100 mL) was added octathiocane (4.32 g, 16.84 mmol, 0.3 eq.) at 25° C. The mixture was stirred at 25° C. for 12 hrs. The reaction mixture was concentrated under reduced pressure, and then the residue was triturated with PE (50 mL) at 25° C. for 30 mins to give crude compound 2 (12.5 g) as a white solid, which was used directly for next step without further purification.

¹HNMR: (400 MHz, DEUTERIUM OXIDE) δ ppm 7.72 (br d, J=7.9 Hz, 2H), 7.29 (br d, J=7.5 Hz, 2H), 2.32 (br s, 3H).

### Procedure for Preparation of Compound 4

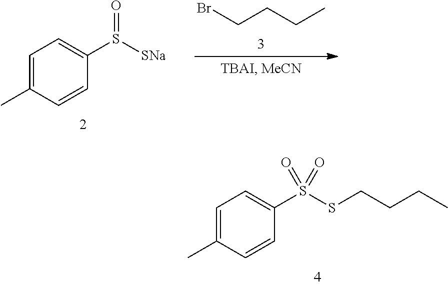

To a mixture of compound 2 (6.3 g, crude, 29.96 mmol, 1 eq.) and compound 3 (8.80 g, 44.95 mmol, 6.93 mL, 1.5 eq.) in MeCN (60 mL) was added TBAI (1.11 g, 3.00 mmol, 0.1 eq.) at 25° C. The mixture was stirred at 80° C. for 3 hrs. The reaction mixture was poured into ice-water (w/w=1/1, 20 mL), and then stirred for 2 mins. The aqueous phase was extracted with DCM (10 mL*3). The combined organic phases were washed with brine (30 mL), dried over anhydrous Na₂SO₄, filtered and concentrated under vacuum. The residue was purified by column chromatography (SiO₂, Petroleum ether/Ethyl acetate=10/1 to 5/1) to give compound 4 (6 g, 81.94% yield) as a yellow oil.

¹HNMR: (400 MHz, CHLOROFORM-d) δ ppm 7.87 (dd, J=8.3, 12.6 Hz, 2H), 7.48-7.34 (m, 2H), 3.08-2.84 (m, 2H), 2.56-2.45 (m, 3H), 1.74-1.61 (m, 2H), 1.40 (qd, J=7.5, 17.7 Hz, 2H), 1.00-0.84 (m, 3H).

### Procedure for Preparation of Compound 5

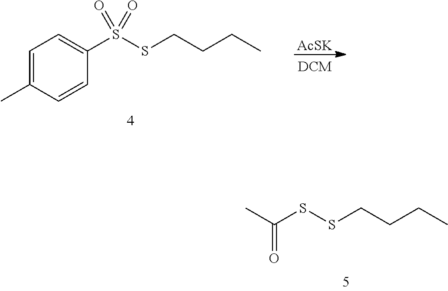

To a mixture of compound 4 (2.6 g, 10.64 mmol, 1 eq.) and AcSK (1.82 g, 15.96 mmol, 1.5 eq.) in DCM (30 mL) was stirred at 25° C. for 12 hrs. The reaction mixture was poured into ice-water (w/w=1/1, 30 mL) and stirred for 2 mins. The aqueous phase was extracted with DCM (20 mL*3). The combined organic phases were washed with brine (30 mL), dried over anhydrous Na₂SO₄, filtered and concentrated under vacuum. The residue was purified by column chromatography (SiO₂, Petroleum ether/Ethyl acetate=20/1 to 10/1) to give compound 5 (1.72 g, 98.40% yield) as a colorless oil.

¹HNMR: (400 MHz, CHLOROFORM-d) δ ppm 2.80-2.69 (m, 2H), 2.47 (s, 3H), 1.69-1.59 (m, 2H), 1.51-1.38 (m, 2H), 0.94 (t, J=7.2 Hz, 3H).

### Procedure for Preparation of Compound 8

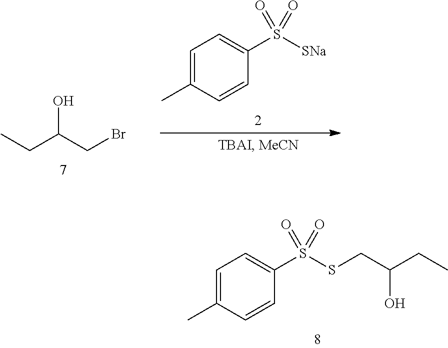

To a mixture of compound 2 (6 g, 28.54 mmol, 1 eq.) and compound 7 (9.36 g, 70% purity, 42.81 mmol, 1.5 eq.) in MeCN (50 mL) was added TBAI (1.05 g, 2.85 mmol, 0.1 eq.) at 25° C. The mixture was stirred at 80° C. for 3 hrs. The reaction mixture was poured into ice-water (w/w=1/1, 20 mL) and stirred for 2 mins. The aqueous phase was extracted with DCM (20 mL*3). The combined organic phases were washed with brine (30 mL), dried over anhydrous Na₂SO₄, filtered and concentrated under vacuum. The residue was purified by column chromatography (SiO₂, Petroleum ether/Ethyl acetate=5/1 to 3/1) to give compound 8 (6.1 g, 82.10% yield) as a light-yellow oil.

¹HNMR: (400 MHz, CHLOROFORM-d) δ ppm 7.86 (d, J=8.1 Hz, 2H), 7.39 (d, J=7.9 Hz, 2H), 3.84-3.74 (m, 1H), 3.20 (dd, J=3.5, 14.1 Hz, 1H), 3.03 (dd, J=8.0, 14.0 Hz, 1H), 2.49 (s, 3H), 2.15 (br d, J=2.4 Hz, 1H), 1.59-1.51 (m, 2H), 0.97 (t, J=7.4 Hz, 3H).

### Procedure for Preparation of Compound 9

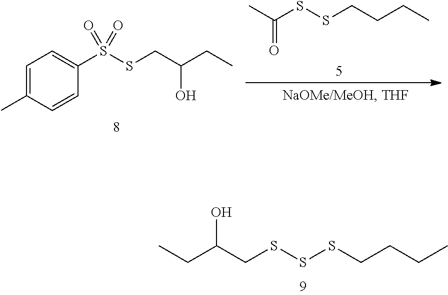

To a mixture of compound 8 (2 g, 7.68 mmol, 1 eq.) and compound 5 (1.39 g, 8.45 mmol, 1.1 eq.) in THF (50 mL) was added NaOMe (1 M MeOH solution, 7.68 mL, 1 eq.) at −78° C. The mixture was stirred at −78° C. for 30 mins. The reaction mixture was poured into ice-water (w/w=1/1, 30 mL) and stirred for 2 mins. The aqueous phase was extracted with DCM (20 mL*3). The combined organic phases were washed with brine (30 mL), dried over anhydrous Na₂SO₄, filtered and concentrated under vacuum. The residue was purified by column chromatography (SiO₂, Petroleum ether/Ethyl acetate=20/1 to 10/1) to give compound 9 (1.5 g, 81.93% yield) as a light-yellow oil.

LCMS: m/z 226.0 [M+1]⁺; ¹HNMR: (400 MHz, CHLOROFORM-d) δ ppm 3.99-3.84 (m, 1H), 3.12 (dd, J=3.2, 13.8 Hz, 1H), 2.96-2.88 (m, 2H), 2.81 (dd, J=8.8, 13.8 Hz, 1H), 2.33 (br s, 1H), 1.81-1.70 (m, 2H), 1.66-1.58 (m, 2H), 1.51-1.40 (m, 2H), 1.02 (t, J=7.5 Hz, 3H), 0.95 (t, J=7.4 Hz, 3H).

### Procedure for Preparation of Compound 10

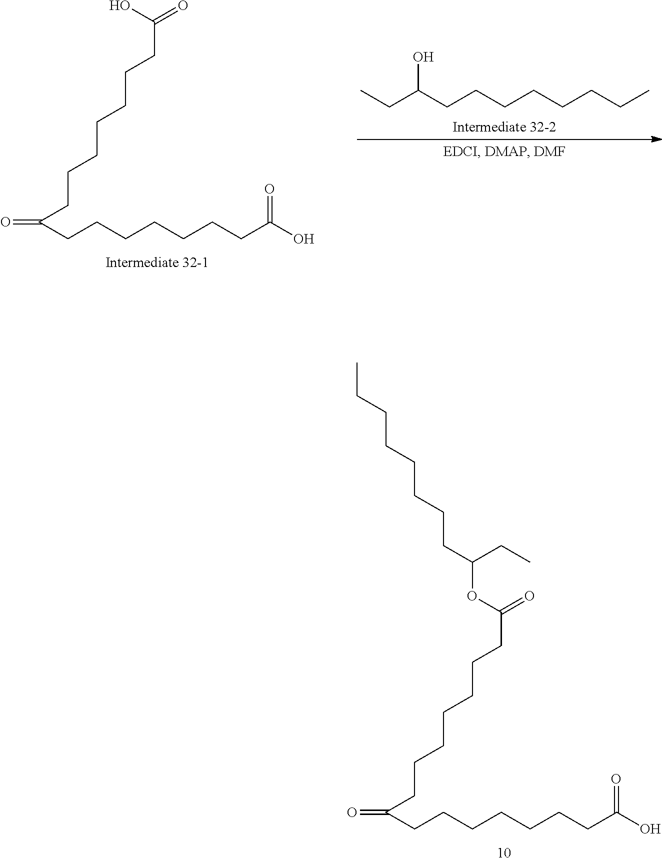 [SMILES: O=C(O)CCCCCCCC(=O)CCCCCCCC(=O)O ; CCCCCCCCC(O)CC ; CCCCCCCCC(CC)OC(=O)CCCCCCCC(=O)CCCCCCCC(=O)O]

To a mixture of intermediate 32-1 (3.56 g, 11.32 mmol, 1.3 eq.) and intermediate 32-2 (1.5 g, 8.71 mmol, 1 eq.) in DMF (30 mL) was added EDCI (2.19 g, 11.40 mmol, 1.31 eq.) and DMAP (1.10 g, 8.97 mmol, 1.03 eq.) at 25° C. The mixture was stirred at 25° C. for 12 hrs. The reaction mixture was poured into ice-water (w/w=1/1, 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether/Ethyl acetate=5/1 to 3/1) to give compound 10 (1 g, 24.51% yield) as a light-yellow oil.

¹H NMR: (400 MHz, CHLOROFORM-d) δ ppm 4.83 (t, J=6.0 Hz, 1H), 2.51-2.24 (m, 8H), 1.71-1.56 (m, 10H), 1.46-1.16 (m, 26H), 1.02-0.78 (m, 6H).

### Procedure for Preparation of Compound 11

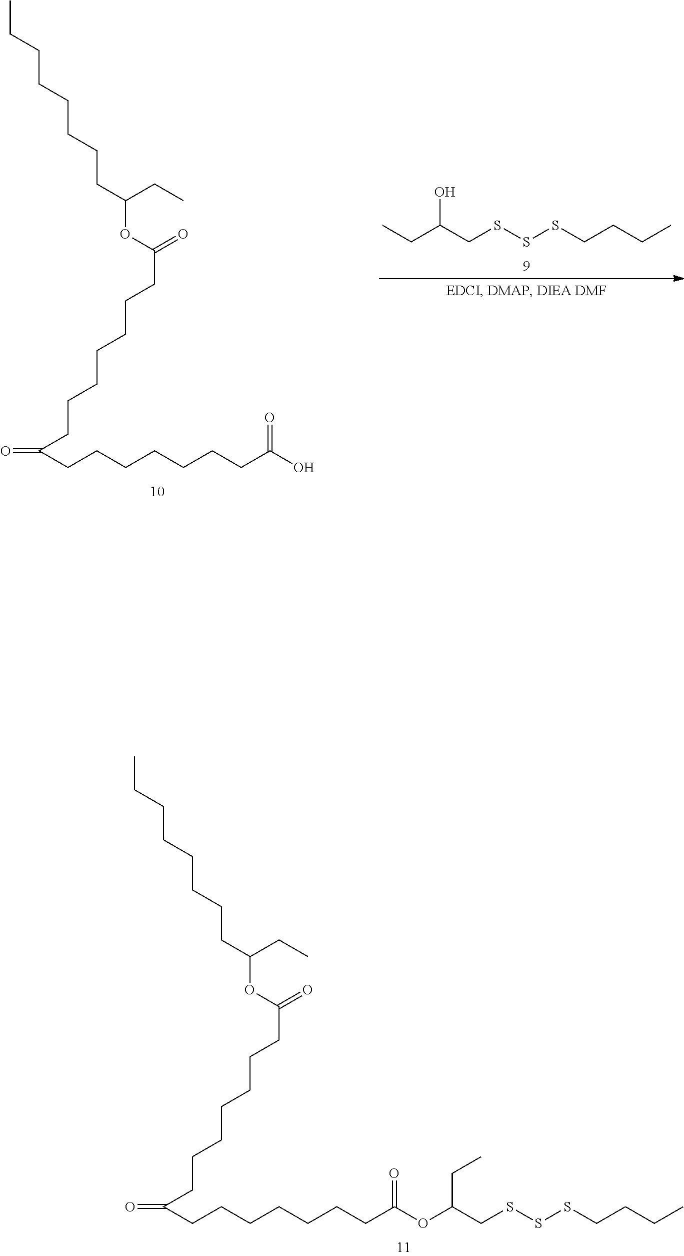 [SMILES: CCCCCCCCC(CC)OC(=O)CCCCCCCC(=O)CCCCCCCC(=O)O ; CCCCSSSCC(O)CC ; CCCCCCCCC(CC)OC(=O)CCCCCCCC(=O)CCCCCCCC(=O)OC(CC)CSSSCCCC]

To a mixture of compound 10 (1 g, 2.13 mmol, 1 eq.) and compound 9 (531.39 mg, 2.35 mmol, 1.1 eq.) in DCM (10 mL) was added EDCI (1.02 g, 5.33 mmol, 2.5 eq.), DIEA (1.38 g, 10.67 mmol, 1.86 mL, 5 eq.) and DMAP (268.47 mg, 2.20 mmol, 1.03 eq.) at 25° C. The mixture was stirred at 40° C. for 12 hrs. The reaction mixture was poured into ice-water (w/w=1/1, 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether/Ethyl acetate=5/1 to 3/1) to give compound 11 (1.3 g, 86.84% yield, 96.5% purity) as a light-yellow oil,

¹H NMR: (400 MHz, CHLOROFORM-d) δ ppm 5.14 (quin, J=6.0 Hz, 1H), 4.84 (t, J=6.0 Hz, 1H), 3.17-3.03 (m, 2H), 2.91 (t, J=7.2 Hz, 2H), 2.46-2.28 (m, 8H), 1.82-1.69 (m, 4H), 1.69-1.60 (m, 6H), 1.58-1.52 (m, 4H), 1.50-1.41 (m, 2H), 1.31 (brd, J=17.2 Hz, 24H), 1.04 (br d, J=3.6 Hz, 2H), 1.01-0.87 (m, 12H).

### Procedure for Preparation of Compound 13

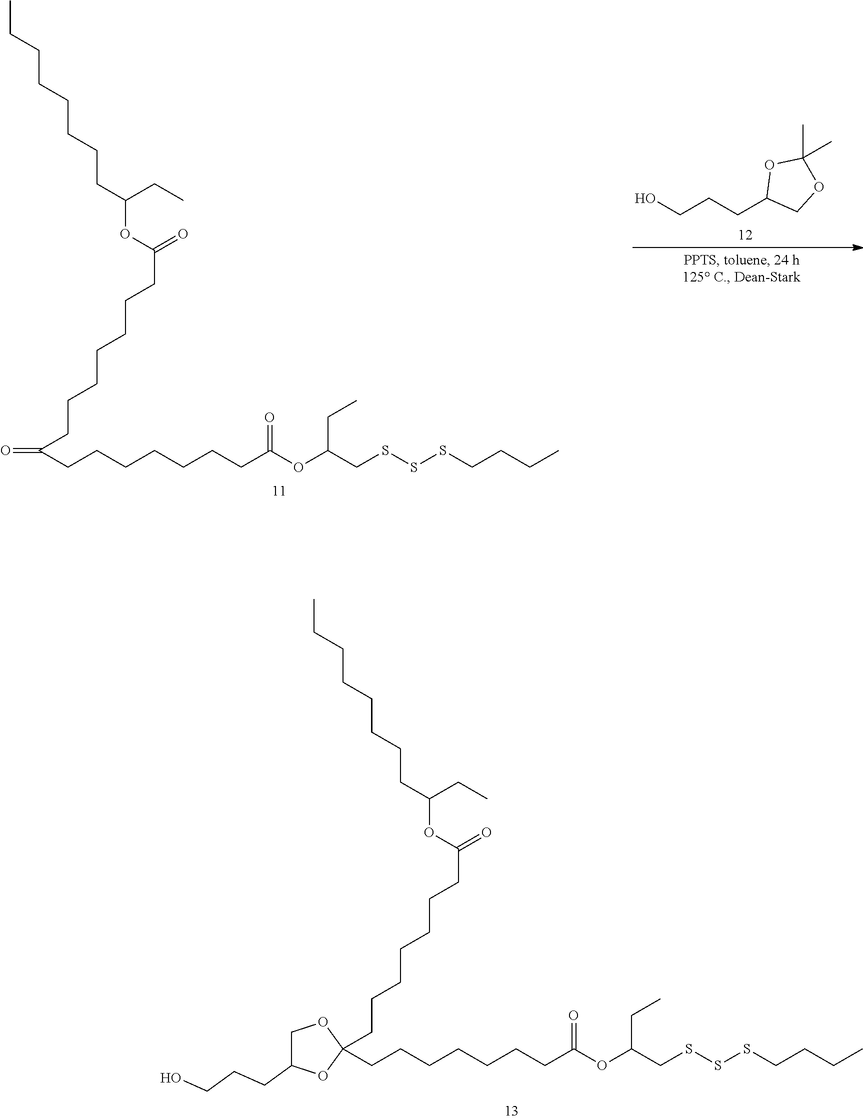 [SMILES: CCCCCCCCC(CC)OC(=O)CCCCCCCC(=O)CCCCCCCC(=O)OC(CC)CSSSCCCC ; CC1(C)OCC(CCCO)O1 ; CCCCCCCCC(CC)OC(=O)CCCCCCCC1(CCCCCCCC(=O)OC(CC)CSSSCCCC)OCC(CCCO)O1]

To a mixture of compound 11 (1.3 g, 1.92 mmol, 1 eq.) and compound 12 (768.97 mg, 4.80 mmol, 2.5 eq.) in toluene (30 mL) was added PPTS (144.74 mg, 575.97 μmol, 0.3 eq.) at 25° C. The mixture was stirred at 130° C. for 12 hrs. The reaction mixture was concentrated under reduced pressure. The residue was purified by column chromatography (SiO₂, Petroleum ether/Ethyl acetate=5/1 to 3/1) to give compound 13 (1.2 g, 74.41% yield, 92.77% purity) as a light-yellow oil.

¹H NMR; (400 MHz, CHLOROFORM-d) δ ppm 5.20-5.06 (m, 1H), 4.83 (quin, J=6.0 Hz, 1H), 4.18-4.04 (m, 2H), 3.71 (br d, J=4.8 Hz, 2H), 3.54-3.43 (m, 1H), 3.14-3.04 (m, 2H), 2.90 (t, J=7.2 Hz, 2H), 2.32 (td, J=7.6, 12.3 Hz, 4H), 2.20-2.08 (m, 1H), 1.82-1.59 (m, 16H), 1.57-1.42 (m, 6H), 1.39-1.26 (m, 28H), 0.99-0.86 (m, 12H).

### Procedure for Preparation of Compound 14

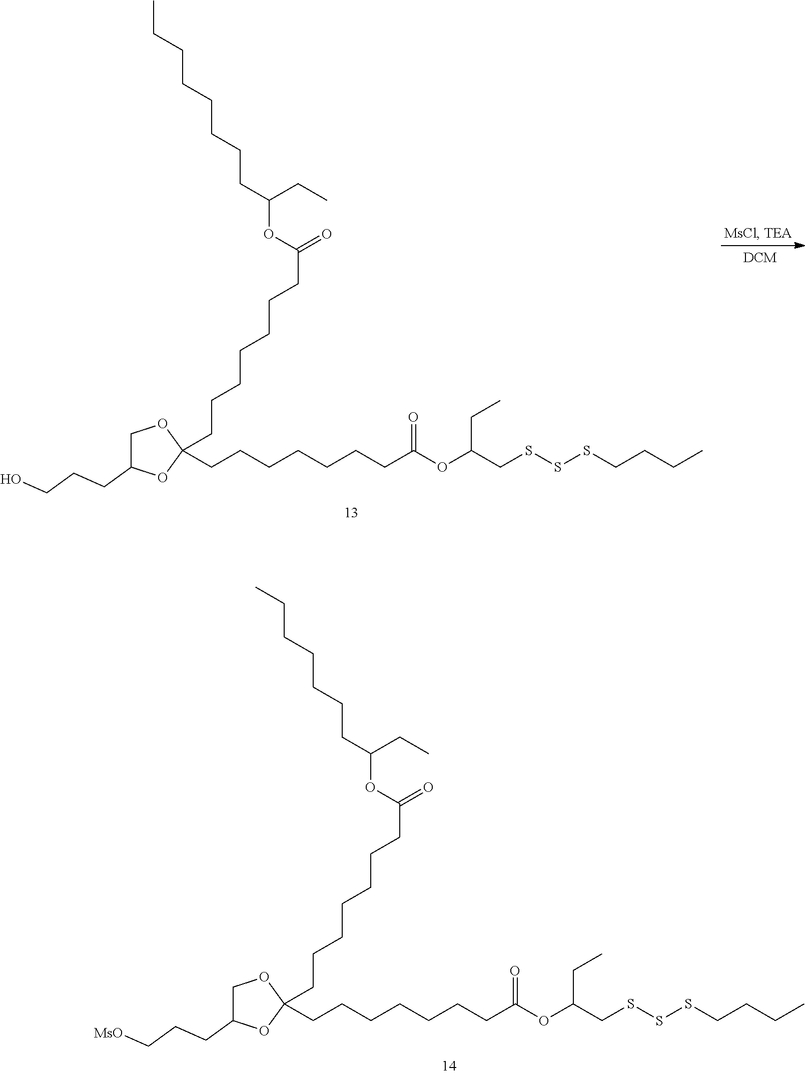 [SMILES: CCCCCCCCC(CC)OC(=O)CCCCCCCC1(CCCCCCCC(=O)OC(CC)CSSSCCCC)OCC(CCCO)O1 ; CCCCCCCC(CC)OC(=O)CCCCCCCC1(CCCCCCCC(=O)OC(CC)CSSSCCCC)OCC(CCCOS(C)(=O)=O)O1]

To a solution of compound 13 (1.2 g, 1.54 mmol, 1 eq.) in DCM (20 mL) was added TEA (779.13 mg, 7.70 mmol, 1.07 mL, 5 eq.) and MsCl (529.21 mg, 4.62 mmol, 357.57 μL, 3 eq.) at 25° C. The mixture was stirred at 25° C. for 2 hrs. The reaction mixture was poured into ice-water (w/w=1/1, 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether/Ethyl acetate=5/1 to 3/1) to give compound 14 (780 mg, 59.08% yield) as a light-yellow oil.

¹H NMR; (400 MHz, CHLOROFORM-d) δ ppm 5.21-5.08 (m, 1H), 4.84 (t, J=6.0 Hz, 1H), 4.41-4.25 (m, 2H), 4.12-4.04 (m, 2H), 3.57-3.44 (m, 1H), 3.14-3.07 (m, 2H), 3.05 (s, 3H), 2.91 (t, J=7.2 Hz, 2H), 2.33 (td, J=7.6, 12.3 Hz, 4H), 2.01-1.83 (m, 2H), 1.81-1.64 (m, 10H), 1.59 (br s, 6H), 1.50-1.42 (m, 2H), 1.40-1.25 (m, 32H), 1.01-0.89 (m, 12H).

### Procedure for Preparation of Lipid 32

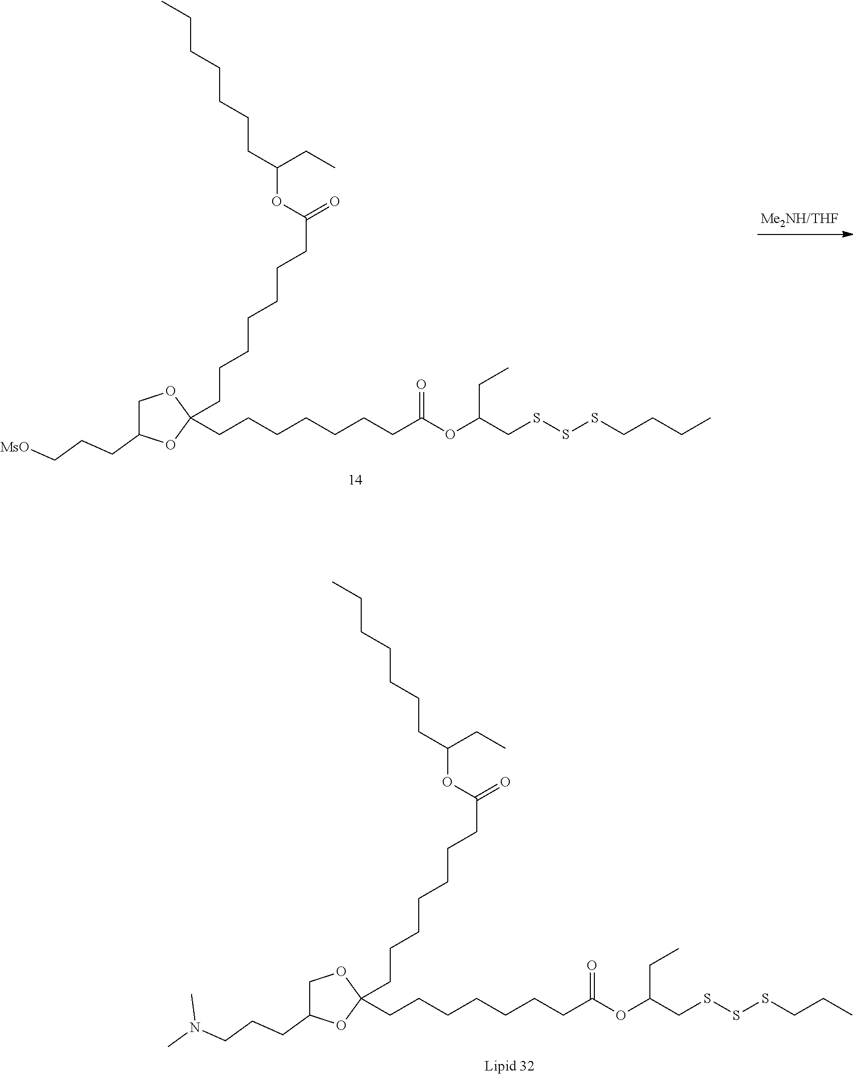

To a solution of compound 14 (700 mg, 816.48 μmol, 1 eq.) in THF (1 mL) was added Me₂NH (2 M THF solution, 11.67 mL, 28.58 eq.) at 25° C. The mixture was stirred at 40° C. for 12 hrs. The reaction mixture was poured into ice-water (w/w=1/1, 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by column chromatography (SiO₂, Petroleum ether/Ethyl acetate=10/1 to 3/1, with 0.1% TEA, followed by prep-HPLC (column: PHS-Phenyl-Hexyl 150*25 mm*7 μm; mobile phase: [water (FA)-MeOH]; gradient: 68%-98% B over 15 min) twice, and then purified by prep-NPLC (column: Welch Ultimate XB—SiOH 250*50*10 μm; mobile phase: [Hexane-EtOH]; B %: 1%, isocratic elution mode) to give Lipid 32 (50 mg, 7.23% yield, 95.15% purity) as a yellow oil.

LCMS: m/z 806.6 [M+1]⁺; ¹H NMR; (400 MHz, CHLOROFORM-d) δ ppm 5.24-5.08 (m, 1H), 4.85 (t, J=6.0 Hz, 1H), 4.17-4.03 (m, 2H), 3.50 (s, 1H), 3.26-3.01 (m, 4H), 2.93 (t, J=7.2 Hz, 2H), 2.86 (s, 2H), 2.82-2.69 (m, 2H), 2.60 (br d, J=7.1 Hz, 6H), 2.34 (td, J=7.6, 12.3 Hz, 4H), 1.83-1.74 (m, 6H), 1.53-1.40 (m, 8H), 1.32 (br d, J=16.0 Hz, 32H), 1.00-0.90 (m, 12H).

### Synthesis of Lipid 33

### Procedure for Preparation of Compound 1

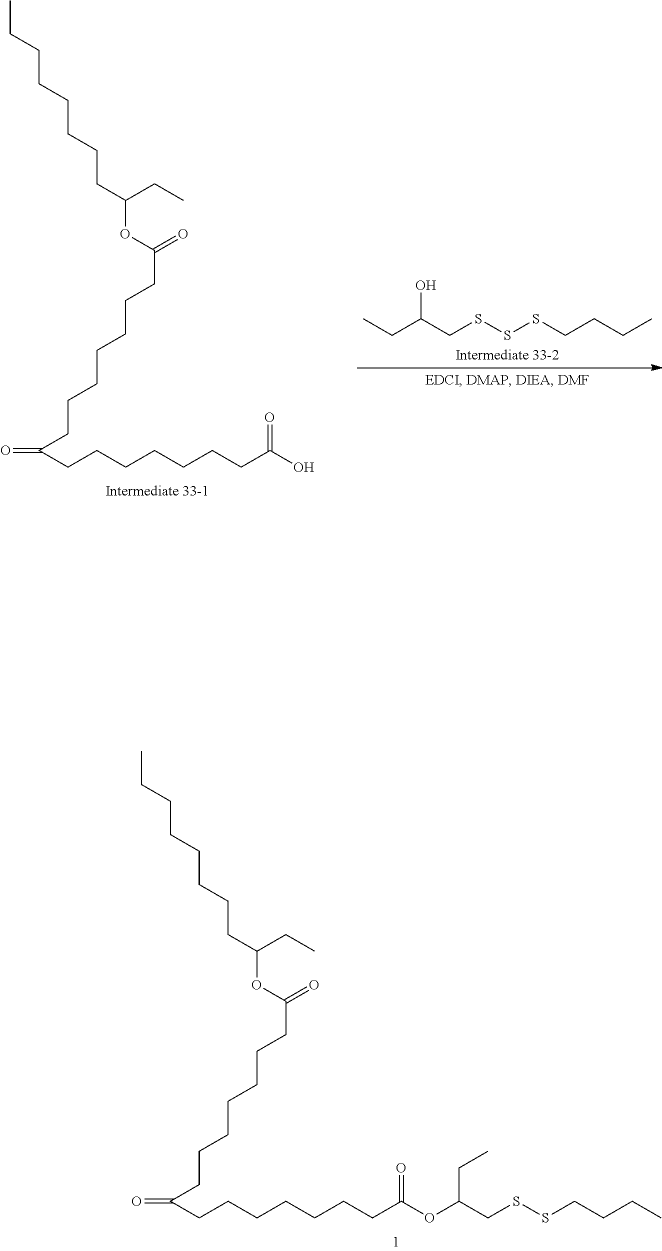 [SMILES: CCCCCCCCC(CC)OC(=O)CCCCCCCC(=O)CCCCCCCC(=O)O ; CCCCSSSCC(O)CC ; CCCCCCCCC(CC)OC(=O)CCCCCCCC(=O)CCCCCCCC(=O)OC(CC)CSSCCCC]

To a mixture of intermediate 33-1 (1 g, 2.13 mmol, 1 eq.) and intermediate 33-2 (483.08 mg, 2.13 mmol, 1 eq.) in DCM (10 mL) was added EDCI (1.02 g, 5.33 mmol, 2.5 eq.), DIEA (1.38 g, 10.67 mmol, 1.86 mL, 5 eq.) and DMAP (268.47 mg, 2.20 mmol, 1.03 eq.) at 25° C. The mixture was stirred at 25° C. for 12 hrs. The reaction mixture was concentrated under reduced pressure. The residue was purified by column chromatography (SiO₂, Petroleum ether/Ethyl acetate=5/1 to 3/1) to give compound 1 (1.4 g, 82.37% yield, 85% purity) as a light-yellow oil.

¹H NMR: (400 MHz, CHLOROFORM-d) δ ppm 5.06 (dd, J=5.6, 7.1 Hz, 1H), 4.83 (t, J=6.0 Hz, 1H), 2.89 (d, J=6.0 Hz, 2H), 2.80-2.67 (m, 2H), 2.45-2.25 (m, 8H), 1.83-1.61 (m, 8H), 1.58-1.50 (m, 8H), 1.48-1.39 (m, 2H), 1.38-1.22 (m, 24H), 0.99-0.86 (m, 12H).

### Procedure for Preparation of Compound 3

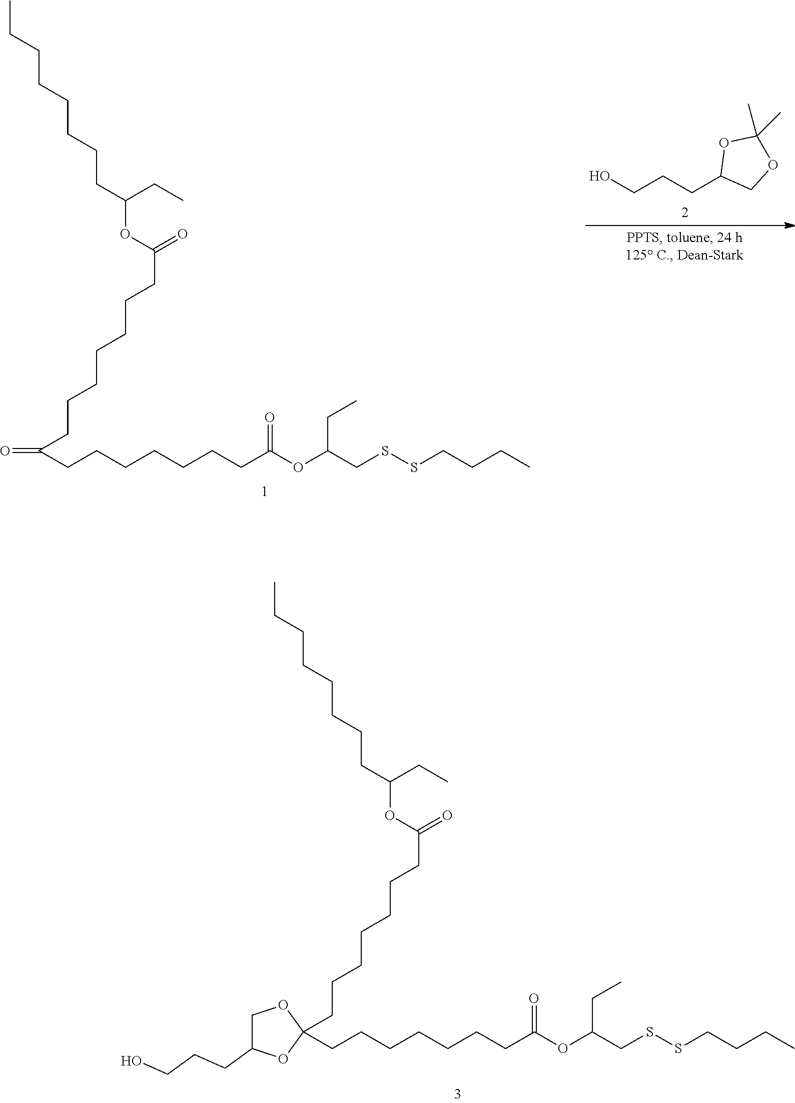 [SMILES: CCCCCCCCC(CC)OC(=O)CCCCCCCC(=O)CCCCCCCC(=O)OC(CC)CSSCCCC ; CC1(C)OCC(CCCO)O1 ; CCCCCCCCC(CC)OC(=O)CCCCCCCC1(CCCCCCCC(=O)OC(CC)CSSCCCC)OCC(CCCO)O1]

To a mixture of compound 1 (1.4 g, 2.07 mmol, 1 eq.) and compound 2 (828.13 mg, 5.17 mmol, 2.5 eq.) in toluene (30 mL) was added PPTS (155.88 mg, 620.28 μmol, 0.3 eq.) at 25° C. The mixture was stirred at 130° C. for 12 hrs. The reaction mixture was concentrated under reduced pressure. The residue was purified by column chromatography (SiO₂, Petroleum ether/Ethyl acetate=20/1 to 3/1) to give compound 3 (1.3 g, 62.94% yield, 78% purity) as a light-yellow oil.

1H NMR: (400 MHz, CHLOROFORM-d) δ ppm 5.19-4.95 (m, 1H), 4.83 (t, J=6.0 Hz, 1H), 4.19-4.03 (m, 2H), 3.71 (q, J=5.2 Hz, 2H), 3.55-3.43 (m, 1H), 2.89 (d, J=6.0 Hz, 2H), 2.80-2.67 (m, 2H), 2.32 (td, J=7.2, 12.3 Hz, 4H), 1.67-1.50 (m, 21H), 1.48-1.40 (m, 2H), 1.37-1.23 (m, 28H), 0.97-0.87 (m, 12H).

### Procedure for Preparation of Compound 4

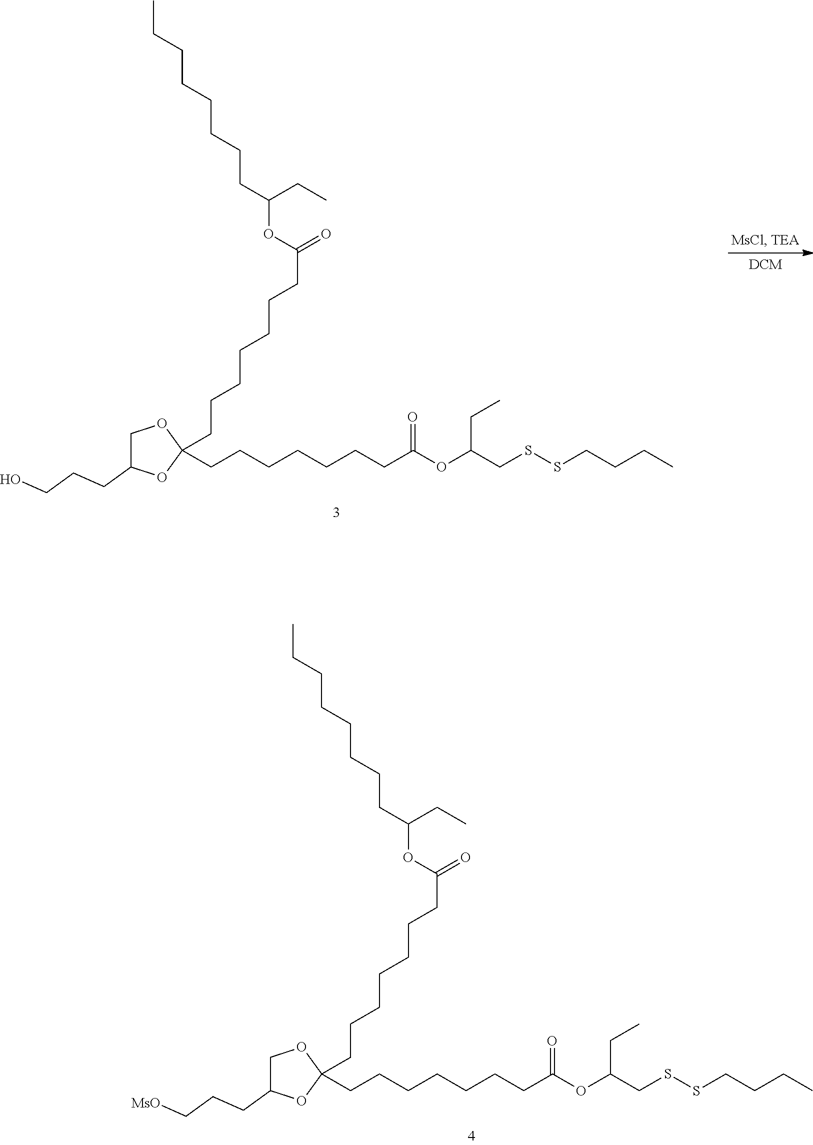 [SMILES: CCCCCCCCC(CC)OC(=O)CCCCCCCC1(CCCCCCCC(=O)OC(CC)CSSCCCC)OCC(CCCO)O1 ; CCCCCCCCC(CC)OC(=O)CCCCCCCC1(CCCCCCCC(=O)OC(CC)CSSCCCC)OCC(CCCOS(C)(=O)=O)O1]

To a solution of compound 3 (1.3 g, 1.67 mmol, 1 eg.) in DCM (15 mL) was added TEA (844.06 mg, 8.34 mmol, 1.16 mL, 5 eq.) and MsCl (573.31 mg, 5.00 mmol, 387.37 μL, 3 eq.) at 25° C. The mixture was stirred at 25° C. for 2 hrs. The reaction mixture was concentrated under reduced pressure. The residue was purified by column chromatography (SiO₂, Petroleum ether/Ethyl acetate=20/1 to 3/1) to give compound 4 (900 mg, 62.92% yield) as a yellow oil.

¹H NMR: (400 MHz, CHLOROFORM-d) δ ppm 5.13-5.00 (m, 1H), 4.83 (t, J=5.6 Hz, 1H), 4.30 (q, J=6.4 Hz, 2H), 4.20-4.00 (m, 2H), 3.56-3.43 (m, 1H), 3.04 (s, 3H), 2.89 (d, J=6.0 Hz, 2H), 2.73 (dt, J=1.2, 7.3 Hz, 2H), 2.32 (td, J=7.6, 12.2 Hz, 4H), 2.00-1.58 (m, 18H), 1.48˜ 1.41 (m, 2H), 1.30 (br d, J=16.0 Hz, 30H), 1.01-0.84 (m, 12H).

### Procedure for Preparation of Lipid 33

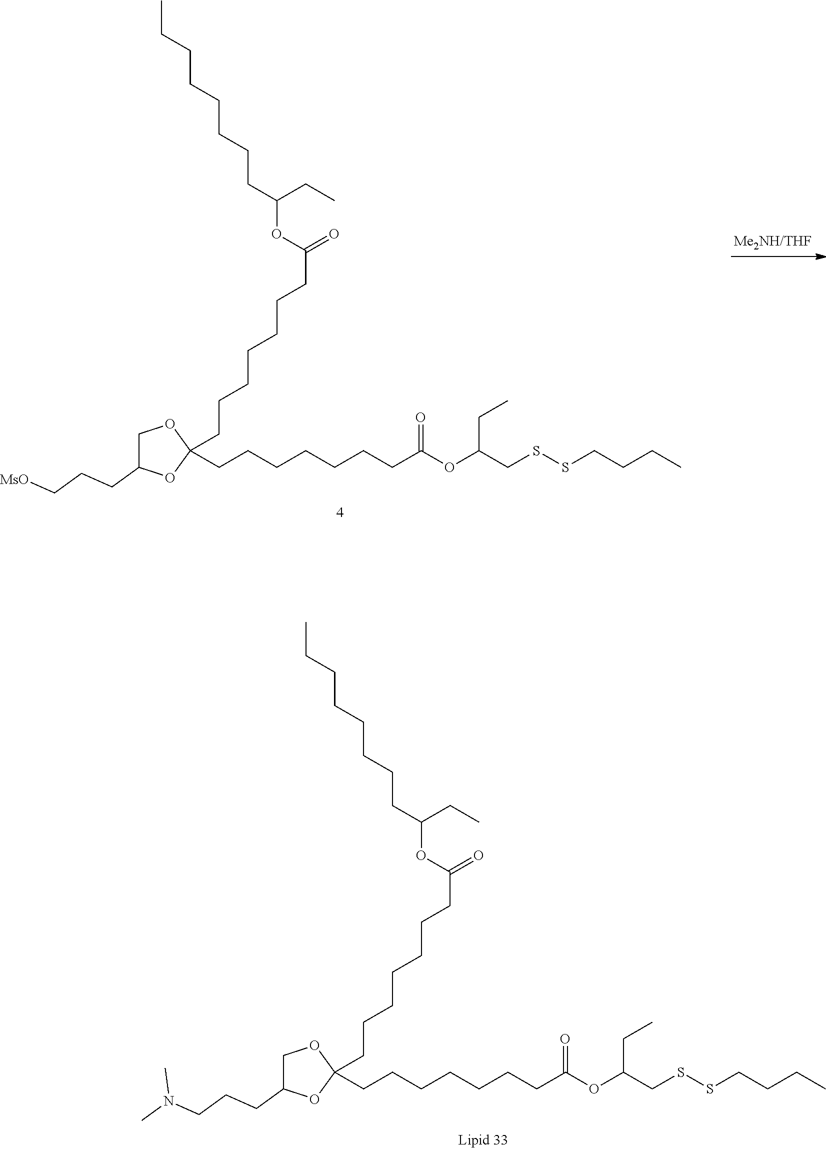

To a solution of compound 4 (900 mg, 1.05 mmol, 1 eq.) in THE (1 mL) was added Me₂NH (2 M THF solution, 6.29 mL, 11.99 eq.) at 25° C. The mixture was stirred at 40° C. for 1 hr. The reaction mixture was concentrated under reduced pressure. The residue was purified by prep-NPLC (column: Welch Ultimate XB—NH₂ 250*50*10 μm; mobile phase: [Hexane-EtOH (0.1% NH₃·H₂O)]; gradient: 1%-15% B over 15 min) twice, followed by prep-HPLC (column: PHS-Phenyl-Hexyl 150*25 mm*7 μm; mobile phase: [water (FA)-MeOH]; gradient: 68%-98% B over 15 min) twice to give Lipid 33 (120 mg, 14.67% yield, 99.39% purity) as a light-yellow oil.

LCMS: m/z 774.6 [M+1]⁺; ¹H NMR: (400 MHz, CHLOROFORM-d) δ ppm 5.17-5.03 (m, 1H), 4.86 (t, J=6.0 Hz, 1H), 4.18-4.02 (m, 2H), 3.57-3.43 (m, 1H), 2.92 (d, J=6.0 Hz, 2H), 2.76 (t, J=7.2 Hz, 2H), 2.45-2.24 (m, 12H), 1.83-1.71 (m, 4H), 1.65-1.23 (m, 46H), 1.06-0.84 (m, 12H).

### Synthesis of Lipid 34

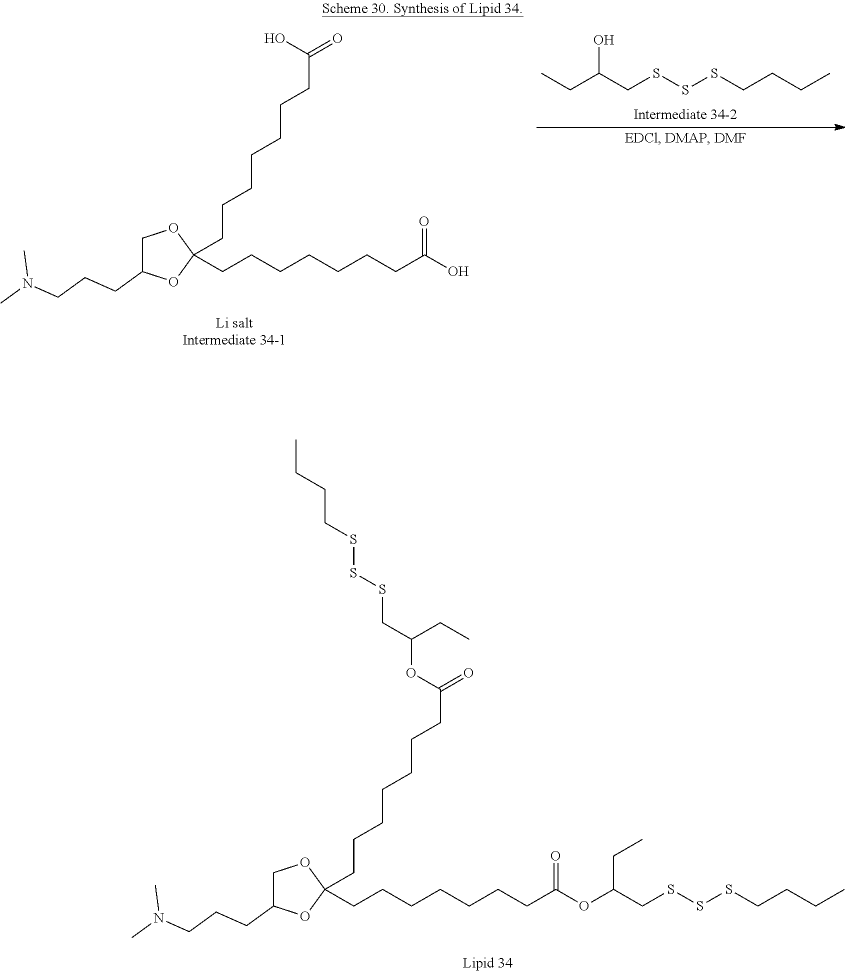 [SMILES: CN(C)CCCC1COC(CCCCCCCC(=O)O)(CCCCCCCC(=O)O)O1 ; CCCCSSSCC(O)CC ; CCCCSSSCC(CC)OC(=O)CCCCCCCC1(CCCCCCCC(=O)OC(CC)CSSSCCCC)OCC(CCCN(C)C)O1]

To a mixture of intermediate 34-1 (300 mg, 676.26 μmol, 1 eq.) and intermediate 34-2 (459.36 mg, 2.03 mmol, 3 eq.) in DMF (6 mL) was added EDCI (648.20 mg, 3.38 mmol, 5 eq.) and DMAP (247.85 mg, 2.03 mmol, 3 eq.) at 25° C. The mixture was stirred at 25° C. for 12 hrs. The reaction mixture was poured into ice-water (w/w=1/1, 30 mL) and stirred for 2 mins. The aqueous phase was extracted with DCM (30 mL*3). The combined organic phases were washed with brine (50 mL), dried over anhydrous Na₂SO₄, filtered and concentrated under vacuum. The residue was purified by prep-HPLC (column: X-Select CSH Phenyl-Hexyl 100*30 5u; mobile phase: [H₂O (0.225% FA)-ACN:THF=2:1]; gradient: 42%-72% B over 15.0 min), followed by prep-HPLC (column: X-Select CSH Phenyl-Hexyl 100*30 5u; mobile phase: [H₂O (0.225% FA)-EtOH]; gradient: 58%-88% B over 15.0 min) to give Lipid 34 (130 mg, 55.96% yield, 99% purity) as a colorless oil.

LCMS: m/z 860.7 [M+1]⁺; ¹H NMR: (400 MHz, CHLOROFORM-d) δ ppm 5.24-5.08 (m, 2H), 4.15-4.05 (m, 2H), 3.53-3.46 (m, 1H), 3.10 (t, J=5.6 Hz, 4H), 2.92 (t, J=7.2 Hz, 4H), 2.85-2.55 (m, 6H), 2.35 (t, J=7.6 Hz, 4H), 1.94-1.62 (m, 20H), 1.55-1.40 (m, 6H), 1.34 (br s, 16H), 1.04-0.89 (m, 12H).

### Synthesis of Lipid 35

### Procedure for Preparation of Compound 4

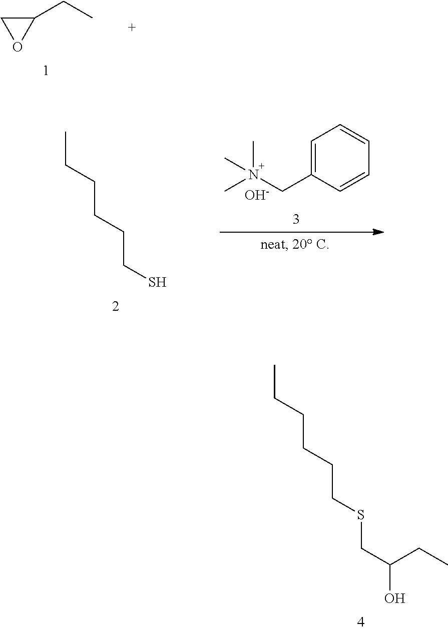

A mixture of compound 1 (2.45 g, 33.98 mmol, 2.96 mL, 1 eq.), compound 2 (4.02 g, 33.98 mmol, 4.78 mL, 1 eq.) and compound 3 (170.48 mg, 1.02 mmol, 185.31 μL, 0.03 eq.) was stirred at 20° C. for 12 hrs. The reaction mixture was directly purified by column chromatography (SiO₂, Petroleum ether/Ethyl acetate=10/1 to 3/1) to give compound 4 (5.3 g, 81.95% yield) as a colourless oil.

¹H NMR: (400 MHz, CHLOROFORM-d) δ ppm 3.69-3.50 (m, 1H), 2.74 (br dd, J=3.2, 13.6 Hz, 1H), 2.66-2.38 (m, 4H), 1.67-1.49 (m, 4H), 1.47-1.22 (m, 7H), 1.11-0.80 (m, 6H).

### Procedure for Preparation of Compound 5

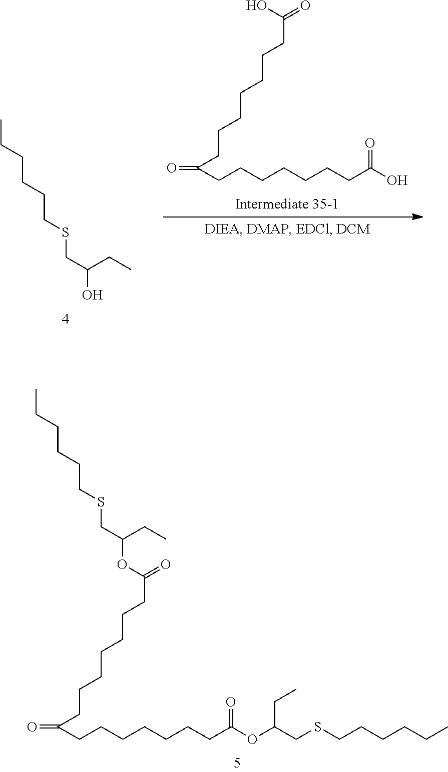 [SMILES: CCCCCCSCC(O)CC ; O=C(O)CCCCCCCC(=O)CCCCCCCC(=O)O ; CCCCCCSCC(CC)OC(=O)CCCCCCCC(=O)CCCCCCCC(=O)OC(CC)CSCCCCCC]

The solution of intermediate 35-1 (1 g, 3.18 mmol, 1 eq.) in DCM (10 mL) was added DIEA (2.06 g, 15.90 mmol, 2.77 mL, 5 eq.), EDCI (1.52 g, 7.95 mmol, 2.5 eq.), DMAP (971.38 mg, 7.95 mmol, 2.5 eq.) and compound 4 (1.33 g, 7.00 mmol, 2.2 eq.) at 25° C. The mixture was stirred at 40° C. for 12 hrs. The reaction mixture was quenched by addition of H₂O (100 mL), and then extracted with EtOAc (100 mL). The organic layer was washed with brine (100 mL), dried over anhydrous Na₂SO₄, filtered and concentrated under reduced pressure. The residue was purified by column chromatography (SiO₂, Petroleum ether/Ethyl acetate=10/1 to 5/1) to give compound 5 (1.2 g, 57.25% yield) as a colourless oil.

¹H NMR: (400 MHz, CHLOROFORM-d) δ ppm 4.97-4.83 (m, 2H), 2.64 (d, J=6.2 Hz, 4H), 2.54 (t, J=7.4 Hz, 4H), 2.37 (t, J=7.4 Hz, 4H), 2.31 (t, J=7.4 Hz, 4H), 1.83-1.70 (m, 2H), 1.69-1.51 (m, 16H), 1.46-1.19 (m, 25H), 0.96-0.83 (m, 12H).

### Procedure for Preparation of Compound 7

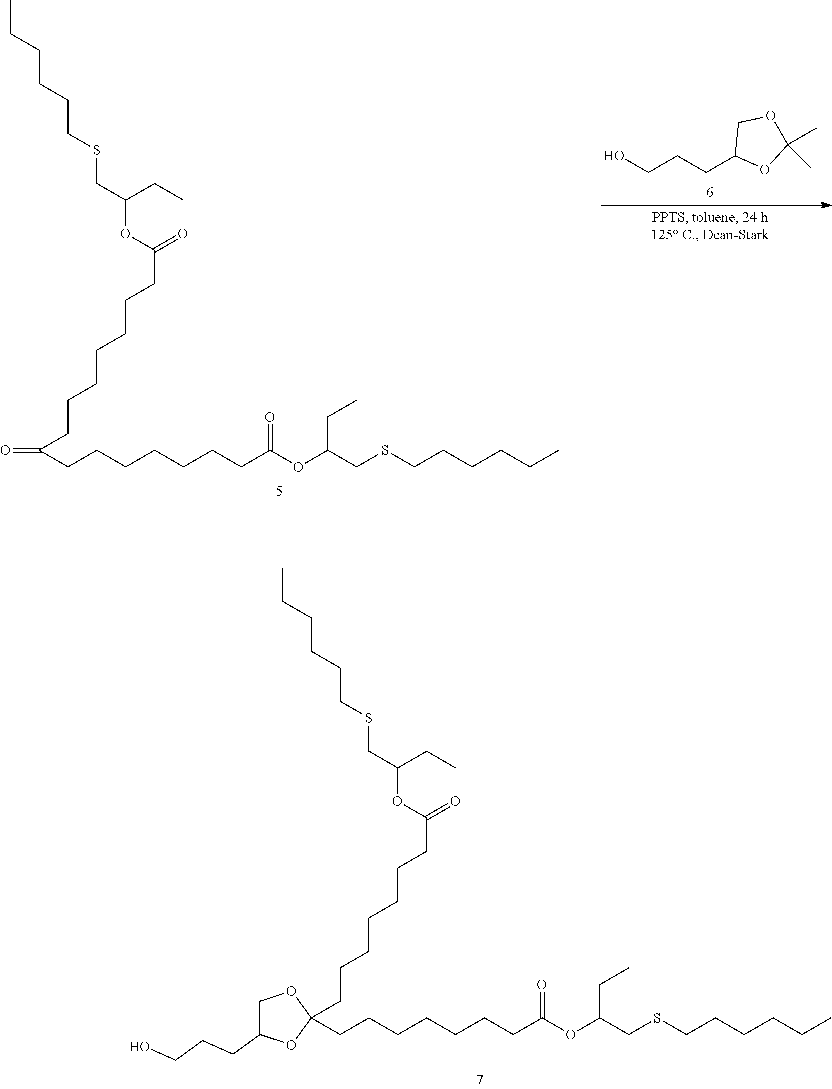 [SMILES: CCCCCCSCC(CC)OC(=O)CCCCCCCC(=O)CCCCCCCC(=O)OC(CC)CSCCCCCC ; CC1(C)OCC(CCCO)O1 ; CCCCCCSCC(CC)OC(=O)CCCCCCCC1(CCCCCCCC(=O)OC(CC)CSCCCCCC)OCC(CCCO)O1]

To a solution of compound 5 (850 mg, 1.29 mmol, 1 eq.) in toluene (40 mL) was added PPTS (97.23 mg, 386.91 μmol, 0.3 eq.) and compound 6 (516.55 mg, 3.22 mmol, 2.5 eq.). The mixture was stirred at 125° C. for 12 hrs equipped with Dean-Stark. The reaction mixture was quenched by addition of H₂O (30 mL), and then extracted with EtOAc (30 mL). The organic layer was washed with brine (30 mL), dried over anhydrous Na₂SO₄, filtered and concentrated under reduced pressure. The residue was purified by column chromatography (SiO₂, Petroleum ether/Ethyl acetate=10/1 to 2/1) to give compound 7 (730 mg, 71.39% yield) as a colourless oil.

¹H NMR: (400 MHz, CHLOROFORM-d) δ ppm 4.97-4.84 (m, 2H), 4.18-4.00 (m, 2H), 3.69 (qd, J=5.0, 9.8 Hz, 2H), 3.54-3.41 (m, 1H), 2.65 (d, J=6.0 Hz, 4H), 2.54 (t, J=7.4 Hz, 4H), 2.31 (t, J=7.4 Hz, 4H), 1.84-1.72 (m, 2H), 1.71-1.52 (m, 19H), 1.41-1.21 (m, 29H), 0.96-0.84 (m, 12H).

### Procedure for Preparation of Compound 9

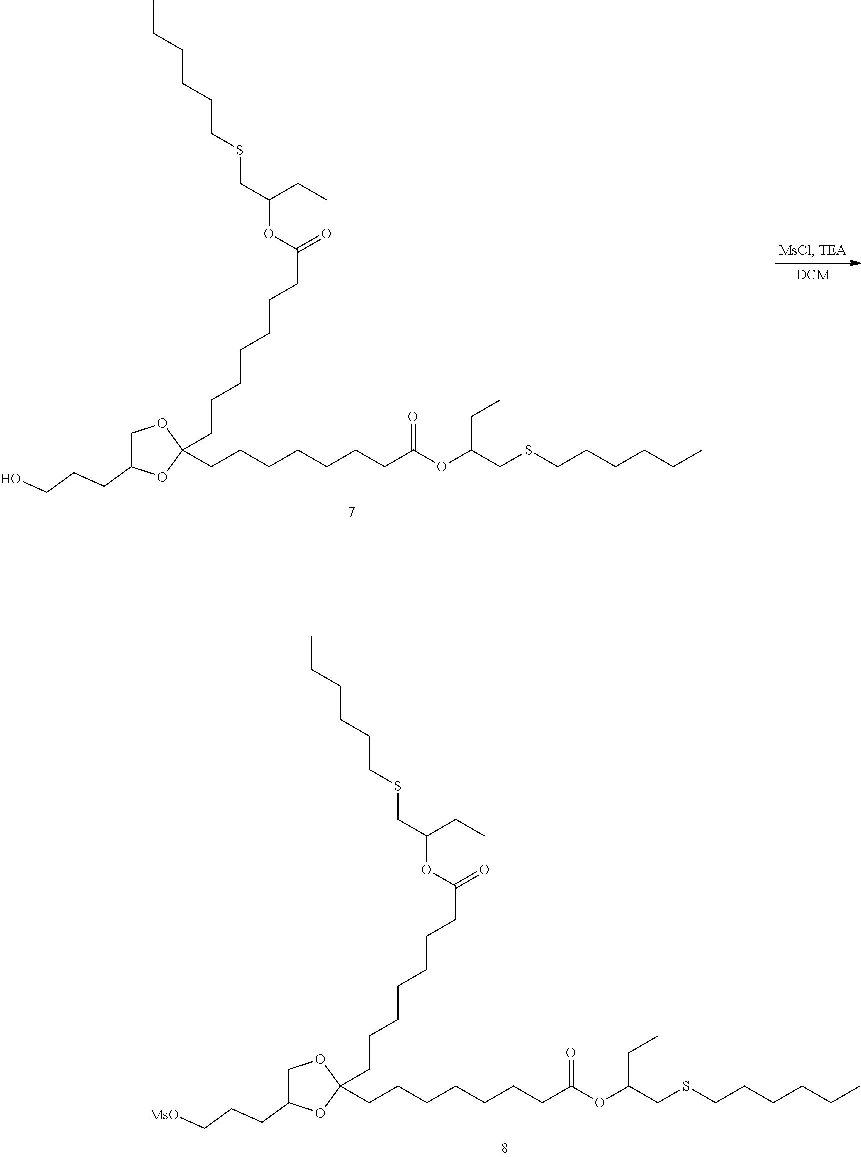 [SMILES: CCCCCCSCC(CC)OC(=O)CCCCCCCC1(CCCCCCCC(=O)OC(CC)CSCCCCCC)OCC(CCCO)O1 ; CCCCCCSCC(CC)OC(=O)CCCCCCCC1(CCCCCCCC(=O)OC(CC)CSCCCCCC)OCC(CCCOS(C)(=O)=O)O1]

To a mixture of compound 7 (720 mg, 945.87 μmol, 1 eq.) and TEA (478.56 mg, 4.73 mmol, 658.26 μL, 5 eq.) in DCM (8 mL) was added MsCl (325.05 mg, 2.84 mmol, 219.63 μL, 3 eq.) dropwise at 0° C. The mixture was stirred at 0° C. for 3 hrs. The reaction mixture was quenched by addition of ice-cold water (10 mL), and then extracted with EtOAc (15 mL*3). The combined organic layers were washed with brine (50 mL), dried over anhydrous Na₂SO₄, filtered and concentrated under reduced pressure. The residue was purified by column chromatography (SiO₂, Petroleum ether/Ethyl acetate=I/O to 0/1) to give compound 8 (640 mg, 75.78% yield) as a colourless oil.

¹H NMR: (400 MHz, CHLOROFORM-d) δ ppm 4.97-4.82 (m, 2H), 4.37-4.21 (m, 2H), 4.09-4.01 (m, 2H), 3.50-3.41 (m, 1H), 3.01 (s, 3H), 2.64 (d, J=6.2 Hz, 4H), 2.54 (t, J=7.2 Hz, 4H), 2.31 (t, J=7.4 Hz, 4H), 1.98-1.71 (m, 4H), 1.70-1.51 (m, 17H), 1.43-1.20 (m, 31H), 0.90 (td, J=7.2, 9.8 Hz, 12H).

### Procedure for Preparation of Lipid 35

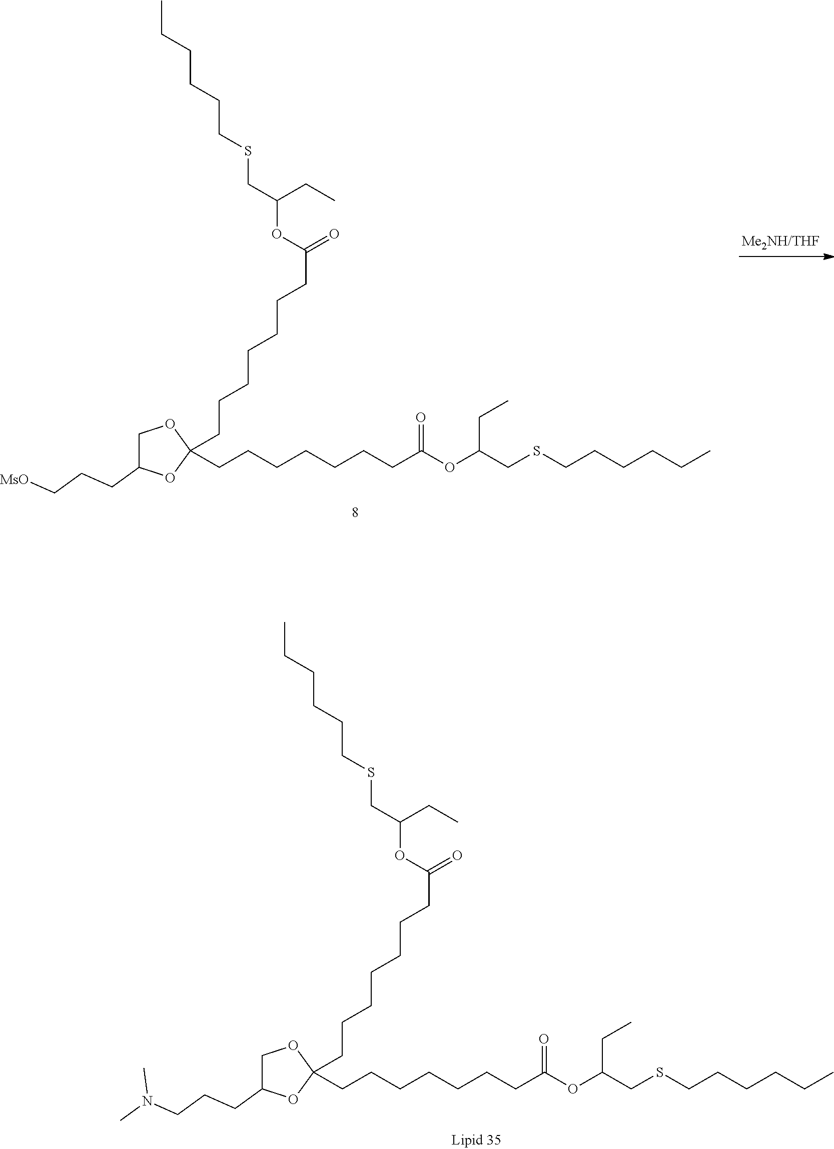

To a solution of compound 8 (640 mg, 762.54 μmol, 1 eq.) in THF (7 mL) was added Me₂NH (2 M THF solution, 1.14 mL, 3 eq.). The mixture was stirred at 50° C. for 12 hrs. The reaction mixture was concentrated under reduced pressure. The residue was purified by column chromatography (SiO₂, Petroleum ether/Ethyl acetate=20/1 to 5/1) to give Lipid 35 as a colourless oil.

LCMS: m/z 788.6 [M+1]}; 1H NMR: (400 MHz, CHLOROFORM-d) δ ppm 4.96-4.83 (m, 2H), 4.12-3.98 (m, 2H), 3.50-3.39 (m, 1H), 2.64 (d, J=6.0 Hz, 4H), 2.54 (t, J=7.4 Hz, 4H), 2.34-2.26 (m, 6H), 2.22 (s, 6H), 1.83-1.70 (m, 2H), 1.68-1.51 (m, 17H), 1.43-1.20 (m, 29H), 0.90 (td, J=7.2, 9.8 Hz, 12H).

### Synthesis of Lipid 36

### Procedure for Preparation of Compound 7

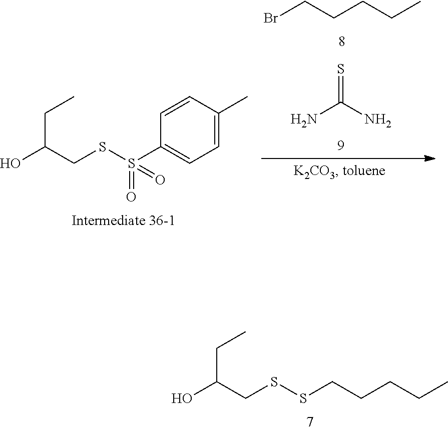

To a mixture of intermediate 36-1 (3 g, 11.52 mmol, 1 eq.), compound 8 (3.48 g, 23.04 mmol, 2.88 mL, 2 eq.) and compound 9 (1.75 g, 23.04 mmol, 2 eq.) in toluene (30 mL) was added K₂CO₃ (1.59 g, 11.52 mmol, 1 eq.). The mixture was stirred at 80° C. for 12 hrs. The reaction mixture was poured into water (50 mL) and extracted with EtOAc (50 mL*2). The combined organic layers were washed with brine (50 mL), dried over anhydrous Na₂SO₄, filtered and concentrated under reduced pressure. The residue was purified by column chromatography (SiO₂, Petroleum ether/Ethyl acetate=I/O to 20/1), followed by prep-HPLC (column: Phenomenex luna C18 150*40 mm*15 μm; mobile phase: [water (FA)-ACN]; gradient: 42%-72% B over 18 min) to give compound 7 (830 mg, 33.3% yield, 99.7% purity) as a yellow oil.

¹H NMR: (400 MHz, CHLOROFORM-d) δ ppm 3.86-3.76 (m, 1H), 2.88 (dd, J=13.6, 3.2 Hz, 1H), 2.76-2.69 (m, 2H), 2.64 (dd, J=13.6, 8.8 Hz, 1H), 2.14 (br d, J=2.0 Hz, 1H), 1.70 (quin, J=7.2 Hz, 2H), 1.63-1.53 (m, 2H), 1.43-1.29 (m, 4H), 1.00 (t, J=7.6 Hz, 3H), 0.95-0.87 (m, 3H).

### Procedure for Preparation of Compound 1

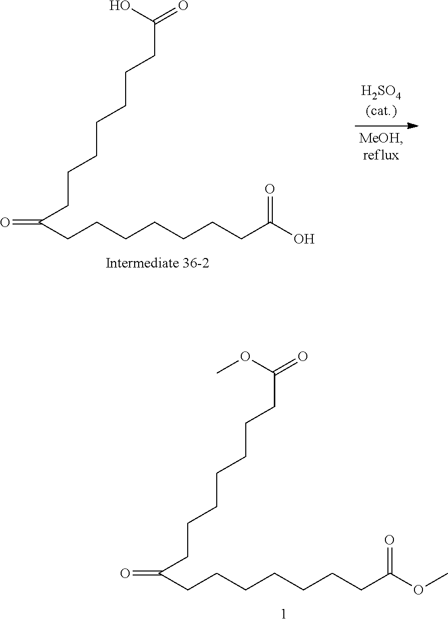 [SMILES: O=C(O)CCCCCCCC(=O)CCCCCCCC(=O)O ; COC(=O)CCCCCCCC(=O)CCCCCCCC(=O)OC]

To a solution of intermediate 36-2 (5 g, 15.90 mmol, 1 eq.) in MeOH (100 mL) was added H₂SO₄ (311.94 mg, 3.18 mmol, 169.53 μL, 0.2 eq.). The mixture was stirred at 65° C. for 6 hrs. The reaction mixture was concentrated under reduced pressure to remove MeOH (retained ˜30 mL), and then poured into ice-water (200 mL). The mixture was filtered, and the filtered cake was dried under vacuum to give crude compound 6 (4.35 g) as a yellow solid.

¹H NMR: (400 MHz, DMSO-d₆) δ ppm 3.57 (s, 6H), 2.37 (t, J=7.2 Hz, 4H), 2.27 (t, J=7.6 Hz, 4H), 1.54-1.38 (m, 8H), 1.27-1.15 (m, 12H).

### Procedure for Preparation of Compound 3

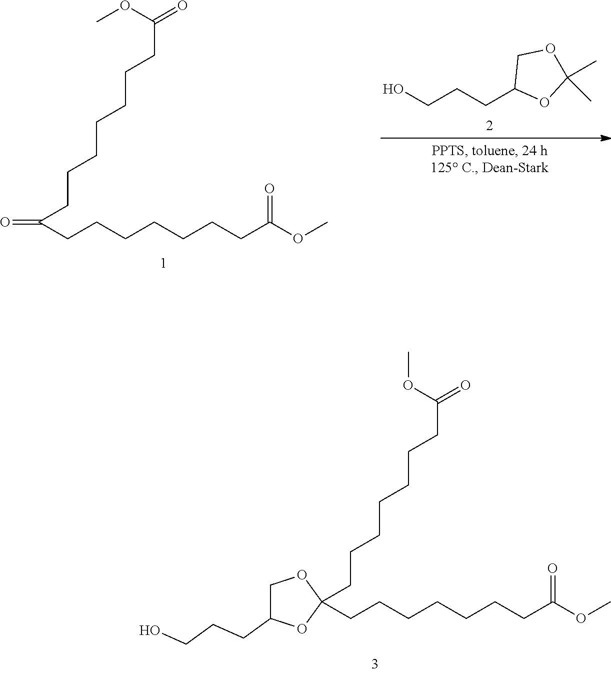 [SMILES: COC(=O)CCCCCCCC(=O)CCCCCCCC(=O)OC ; CC1(C)OCC(CCCO)O1 ; COC(=O)CCCCCCCC1(CCCCCCCC(=O)OC)OCC(CCCO)O1]

To a mixture of compound 1 (2.5 g, 7.30 mmol, 1 eq.) and compound 2 (2.92 g, 18.25 mmol, 2.5 eq.) in toluene (50 mL) was added PPTS (917.24 mg, 3.65 mmol, 0.5 eq.). The mixture was stirred at 135° C. for 12 hrs. After cooled to 25° C., the reaction mixture was poured into ice-water (60 mL), and then extracted with EtOAc (60 mL*3). The combined organic layers were washed with brine (80 mL), dried over anhydrous Na₂SO₄, filtered and concentrated under reduced pressure. The residue was purified by column chromatography (SiO₂, Petroleum ether/Ethyl acetate=I/O to 3/1) to give compound 3 (2.8 g, 52.3% yield, 60.6% purity) as a yellow oil.

¹H NMR: (400 MHz, CHLOROFORM-d) δ ppm 4.15-4.05 (m, 2H), 3.66 (s, 8H), 3.49-3.43 (m, 1H), 2.30 (t, J=7.6 Hz, 4H), 2.20 (br s, 1H), 1.67 (br d, J=4.0 Hz, 4H), 1.63-1.54 (m, 8H), 1.30 (br s, 16H).

### Procedure for Preparation of Compound 4

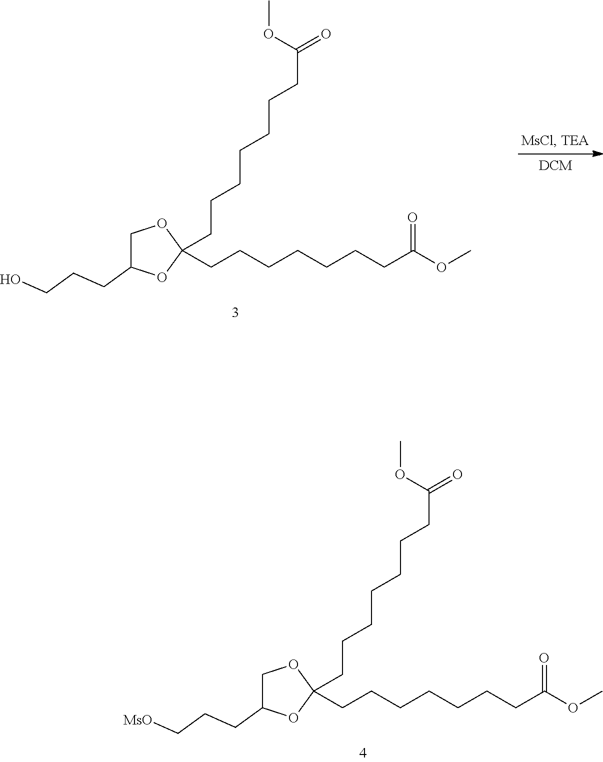 [SMILES: COC(=O)CCCCCCCC1(CCCCCCCC(=O)OC)OCC(CCCO)O1 ; COC(=O)CCCCCCCC1(CCCCCCCC(=O)OC)OCC(CCCOS(C)(=O)=O)O1]

To a mixture of compound 3 (2.8 g, 6.30 mmol, 1 eq.) and TEA (3.19 g, 31.49 mmol, 4.38 mL, 5 eq.) in DCM (28 mL) was added MsCl (2.16 g, 18.89 mmol, 1.46 mL, 3 eq.) dropwise at 0° C. The mixture was stirred at 25° C. for 1 hr. The reaction mixture was poured into ice-water (50 mL), and then extracted with DCM (50 mL*3). The combined organic layers were dried over anhydrous Na₂SO₄, filtered and concentrated under reduced pressure. The residue was purified by column chromatography (SiO₂, Petroleum ether/Ethyl acetate=I/O to 2/1) to give compound 4 (2.39 g, 56.3% yield, 77.6% purity) as a yellow oil.

¹H NMR: (400 MHz, CHLOROFORM-d) δ ppm 4.34-4.23 (m, 2H), 4.10-4.01 (m, 2H), 3.66 (s, 6H), 3.50-3.43 (m, 1H), 3.01 (s, 3H), 2.30 (t, J=7.6 Hz, 4H), 1.76-1.46 (m, 12H), 1.37-1.27 (m, 16H).

### Procedure for Preparation of Compound 5

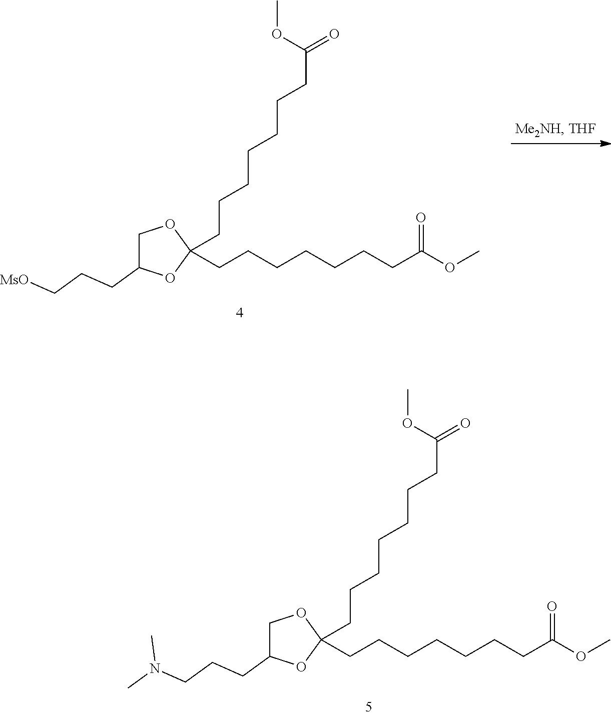

A mixture of compound 4 (2.39 g, 4.57 mmol, 1 eq.) in Me₂NH (2 M THF solution, 159.33 mL, 69.69 eq.) was stirred at 40° C. for 12 hrs. The reaction mixture was poured into water (40 mL) and extracted with EtOAc (40 mL*3). The combined organic layers were washed with brine (50 mL), dried over anhydrous Na₂SO₄, filtered and concentrated under reduced pressure. The residue was purified by column chromatography (SiO₂, DCM/MeOH=100/1 to 10/1) to give compound 5 (1.42 g, 61.7% yield, 93.7% purity) as a yellow oil.

LCMS: m/z 472.4 [M+1]⁺; ¹H NMR: (400 MHz, CHLOROFORM-d) δ ppm 4.08-3.99 (m, 2H), 3.66 (s, 6H), 3.48-3.41 (m, 1H), 2.34-2.27 (m, 6H), 2.24 (s, 6H), 1.67-1.46 (m, 12H), 1.37-1.26 (m, 16H).

### Procedure for Preparation of Compound 6

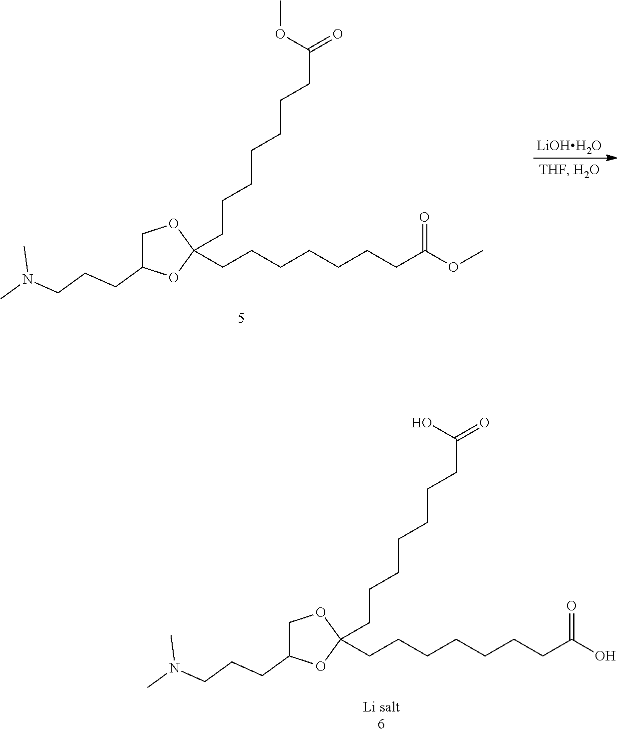

To a solution of compound 5 (1.42 g, 3.01 mmol, 1 eq.) in a mixed solvent of THF (12 mL) and H₂O (4 mL) was added LiOH·H₂O (379.01 mg, 9.03 mmol, 3 eq.). The mixture was stirred at 25° C. for 12 hrs. The reaction mixture was concentrated under reduced pressure to remove THE. The residue was directly freeze-dried to give crude compound 6 (1.35 g, Li salt) as a yellow solid, which was used directly for next step without further purification.

LCMS: m/z 444.4 [M+1]⁺; ¹H NMR: (400 MHz, METHANOL-d₄) δ ppm 4.13-4.01 (m, 2H), 3.45 (t, J=7.2 Hz, 1H), 2.35 (br t, J=6.8 Hz, 2H), 2.24 (s, 6H), 2.15 (t, J=7.6 Hz, 4H), 1.67-1.50 (m, 12H), 1.32 (br s, 16H).

### Procedure for Preparation of Lipid 36

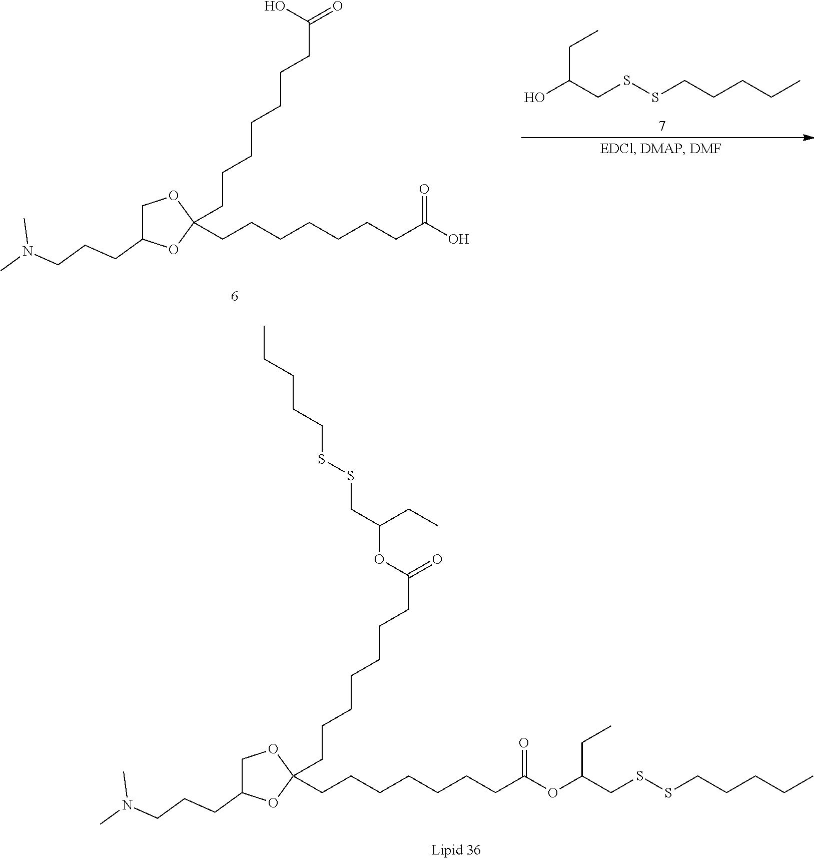 [SMILES: CN(C)CCCC1COC(CCCCCCCC(=O)O)(CCCCCCCC(=O)O)O1 ; CCCCCSSCC(O)CC ; CCCCCSSCC(CC)OC(=O)CCCCCCCC1(CCCCCCCC(=O)OC(CC)CSSCCCCC)OCC(CCCN(C)C)O1]

To a mixture of compound 6 (200 mg, 450.84 μmol, 1 eq.) and compound 7 (281.84 mg, 1.35 mmol, 3 eq.) in DMF (5 mL) was added EDCI (432.13 mg, 2.25 mmol, 5 eq.) and DMAP (165.23 mg, 1.35 mmol, 3 eq.). The mixture was stirred at 25° C. for 12 hrs. The reaction mixture was poured into water (25 mL) and extracted with EtOAc (20 mL*3). The combined organic layers were washed with brine (30 mL), dried over anhydrous Na₂SO₄, filtered and concentrated under reduced pressure. The residue was purified by column chromatography (SiO₂, (Petroleum ether: DCM=3:1)/Ethyl acetate=I/O to 10/1, 0.1% TEA as modifier), followed by prep-HPLC (column: X-Select CSH Phenyl-Hexyl 100*30 5u; mobile phase: [H₂O) (0.225% FA)-MeOH]; gradient: 68%-98% B over 15.0 min), and then purified by prep-HPLC (column: X-Select CSH Phenyl-Hexyl 100*30 5u; mobile phase: [H₂O (0.225% FA)-ACN:THF=2:1]; gradient: 45%-75% B over 15.0 min) to give Lipid 36 (78 mg, 20.0% yield, 95.0% purity) as a yellow oil.

HRMS: m/z 824.5032 [M+1]⁺; ¹H NMR: (400 MHz, CHLOROFORM-d) δ ppm 5.04 (quin, J=6.0 Hz, 2H), 4.11-4.00 (m, 2H), 3.50-3.41 (m, 1H), 2.87 (d, J=6.0 Hz, 4H), 2.71 (t, J=7.2 Hz, 4H), 2.35-2.27 (m, 6H), 2.23 (s, 6H), 1.78-1.50 (m, 20H), 1.40-1.26 (m, 24H), 0.96-0.87 (m, 12H).

### Synthesis of Lipid 37. Lipid 38, and Lipid 39

### Procedure for Preparation of Compound 2

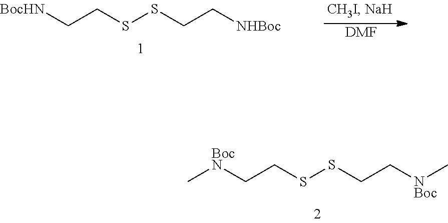

To a solution of compound 1 (5 g, 14.18 mmol, 1 eq.) in DMF (60 mL) was added CH₃₁ (4.83 g, 34.04 mmol, 2.12 mL, 2.4 eq.) and NaH (1.25 g, 31.20 mmol, 60% purity, 2.2 eq.) at 0° C. The mixture was stirred at 25° C. for 2 hours. The mixture was poured into water (500 mL) at 0° C. and extracted with EtOAc (200 mL*3). The organic layers were washed with brine (30 mL*2), dried over anhydrous Na₂SO₄, filtered and concentrated in vacuo. The residue was purified by column chromatography (SiO₂, Petroleum ether/Ethyl acetate=I/O to 3/1) to give compound 2 (3.04 g, 56.32% yield) as a colorless oil.

¹H NMR (400 MHz, CHLOROFORM-d) δ ppm 3.50 (br s, 4H), 2.89 (s, 6H), 2.82 (br s, 4H), 1.46 (s, 18H).

### Procedure for Preparation of Compound 4

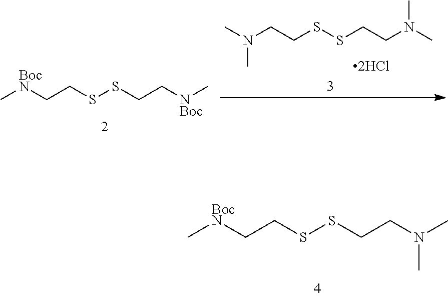

To a solution of compound 3 (960.94 mg, 3.42 mmol, 1 eq., 2 HCl salt) and compound 2 (1.3 g, 3.42 mmol, 1 eq.) in MeOH (6.5 mL) was added a solution of 5.4 M NaOMe MeOH solution (6.33 mL, 10 eq.). The mixture was stirred at 25° C. for 12 hours. The reaction mixture was filtered and concentrated under reduced pressure. The residue was purified by column chromatography (SiO₂, Petroleum ether/Ethyl acetate=1/0˜1/1˜0/1) to give compound 4 (500 mg, 48.71% yield, 98% purity) as a yellow oil.

LCMS: m/z 295.2 [M+1]; 1H NMR (400 MHz, CHLOROFORM-d) δ ppm 3.54-3.43 (m, 2H), 2.89 (s, 3H), 2.85-2.77 (m, 4H), 2.64-2.58 (m, 2H), 2.27 (s, 6H), 1.46 (s, 9H).

### Procedure for Preparation of Compound 5

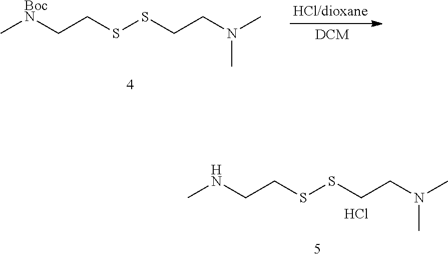

To a solution of compound 4 (500 mg, 1.70 mmol, 1 eq.) in DCM (2.7 mL) was added HCl/dioxane (2 M, 2.50 mL, 2.94 eq.). The mixture was stirred at 25° C. for 16 hours. The reaction mixture was concentrated under vacuum to give crude compound 5 (475 mg, HCl salt) as a white solid, which was used directly for next step without further purification.

¹H NMR (400 MHz, DMSO-d₆) δ ppm 10.10-9.86 (m, 1H), 8.37 (br s, 1H), 2.51-2.50 (m, 6H), 2.17 (br s, 4H), 1.90-1.86 (m, 3H), 1.69-1.64 (m, 2H), 1.63-1.59 (m, 2H)

### Procedure for Preparation of Lipid 37

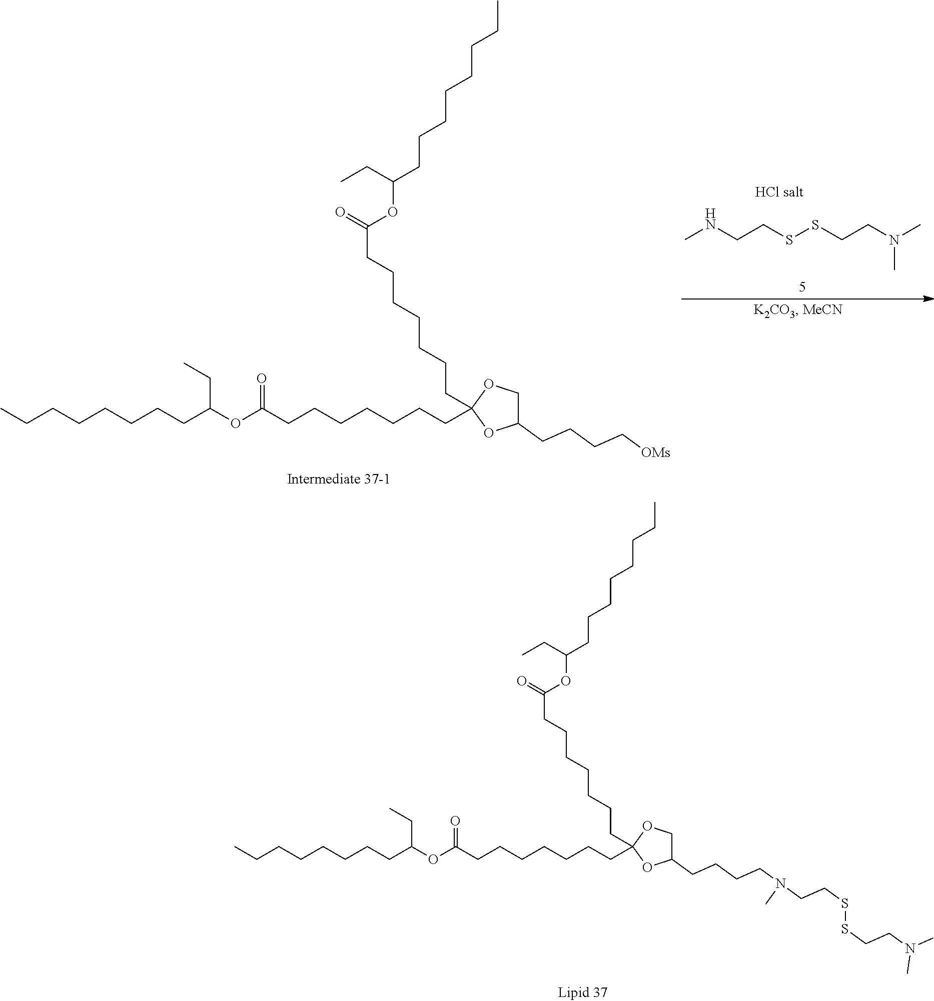

To a solution of compound 5 (127.10 mg, 550.63 μmol, 1.5 eq., HCl salt) and intermediate 37-1 (300 mg, 367.09 μmol, 1 eq.) in MeCN (4 mL) was added K₂CO₃ (152.20 mg, 1.10 mmol, 3 eq.). The mixture was stirred at 90° C. for 16 hours. The reaction mixture was filtered and the filtrate was concentrated under reduced pressure. The residue was purified by column chromatography (SiO₂, Petroleum ether/Ethyl acetate=I/O to 0/1), followed by prep-HPLC (column: PHS-Phenyl-Hexyl 150*25 mm*7 μm; mobile phase: [water (TFA)-MeOH]; gradient: 68%-98% B over 15 min) to give Lipid 37 (43.4 mg, 21.27% yield) as a colorless oil.

HRMS: m/z 915.7273 [M+1]⁺; ¹H NMR (400 MHz, CHLOROFORM-d) δ ppm 4.82 (quin, J=6.0 Hz, 2H), 4.03 (br d, J=3.6 Hz, 2H), 3.48-3.40 (m, 1H), 2.88-2.81 (m, 4H), 2.76 (br d, J=7.2 Hz, 2H), 2.71-2.64 (m, 2H), 2.45 (br s, 2H), 2.34-2.27 (m, 12H), 1.66-1.50 (m, 20H), 1.36-1.23 (m, 44H), 0.91-0.85 (m, 12H).

### Procedure for Preparation of Lipid 38

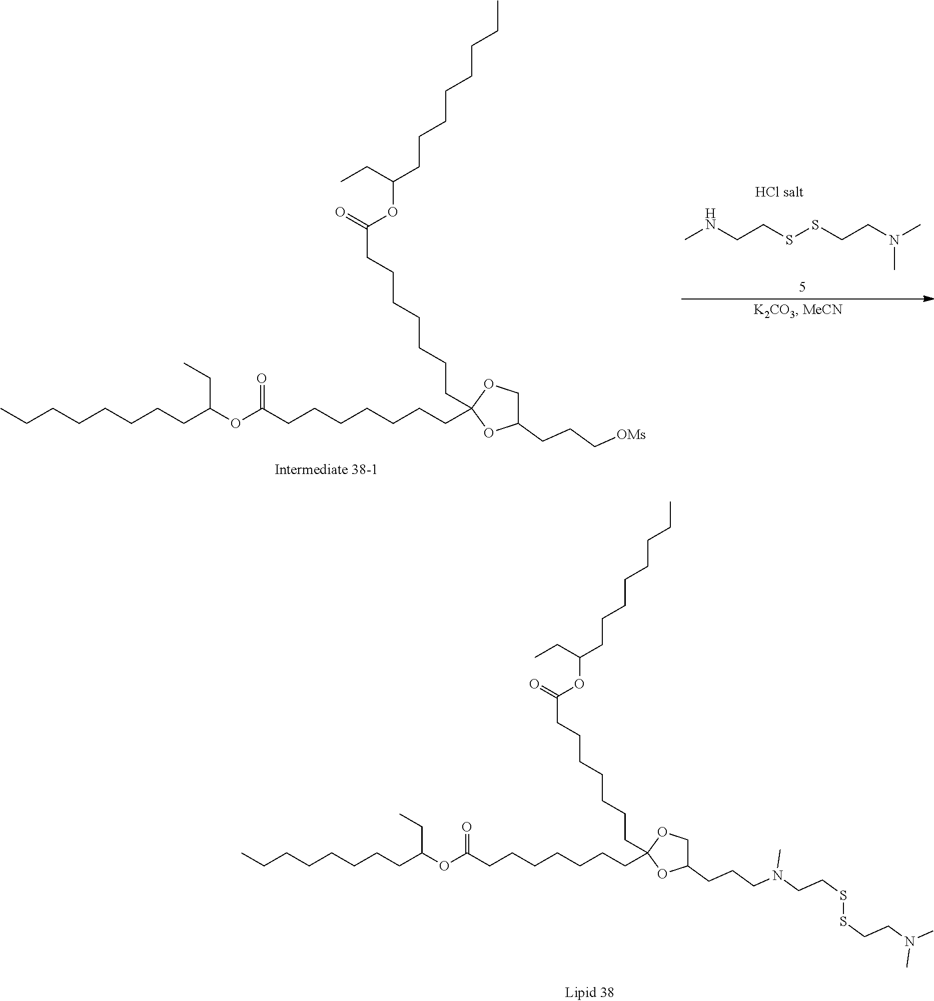

To a solution of compound 5 (129.32 mg, 560.24 μmol, 1.5 eq., HCl salt) in MeCN (4 mL) was added K₂CO₃ (154.86 mg, 1.12 mmol, 3 eq.) and intermediate 38-1 (300 mg, 373.50 μmol, 1 eq.). The mixture was stirred at 90° C. for 16 hours. The reaction mixture was filtered and the filtrate was concentrated under reduced pressure. The residue was purified by prep-HPLC (column: PHS-Phenyl-Hexyl 150*25 mm*7 μm; mobile phase: [water (TFA)-ACN]; gradient: 48%-78% B over 15 min) to give Lipid 38 (36 mg, 10.16% yield, 95% purity) as a colorless oil.

LCMS: m/z 901.8 [M+1]⁺; ¹H NMR (400 MHz, CHLOROFORM-d) δ ppm 4.82 (quin, J=6.4 Hz, 2H), 4.08-4.01 (m, 2H), 3.48-3.42 (m, 1H), 2.88-2.79 (m, 4H), 2.74-2.61 (m, 4H), 2.47-2.38 (m, 2H), 2.32-2.26 (m, 12H), 1.85-1.69 (m, 6H), 1.61-1.49 (m, 16H), 1.33-1.22 (m, 39H), 0.91-0.85 (m, 12H).

### Procedure for Preparation of Lipid 39

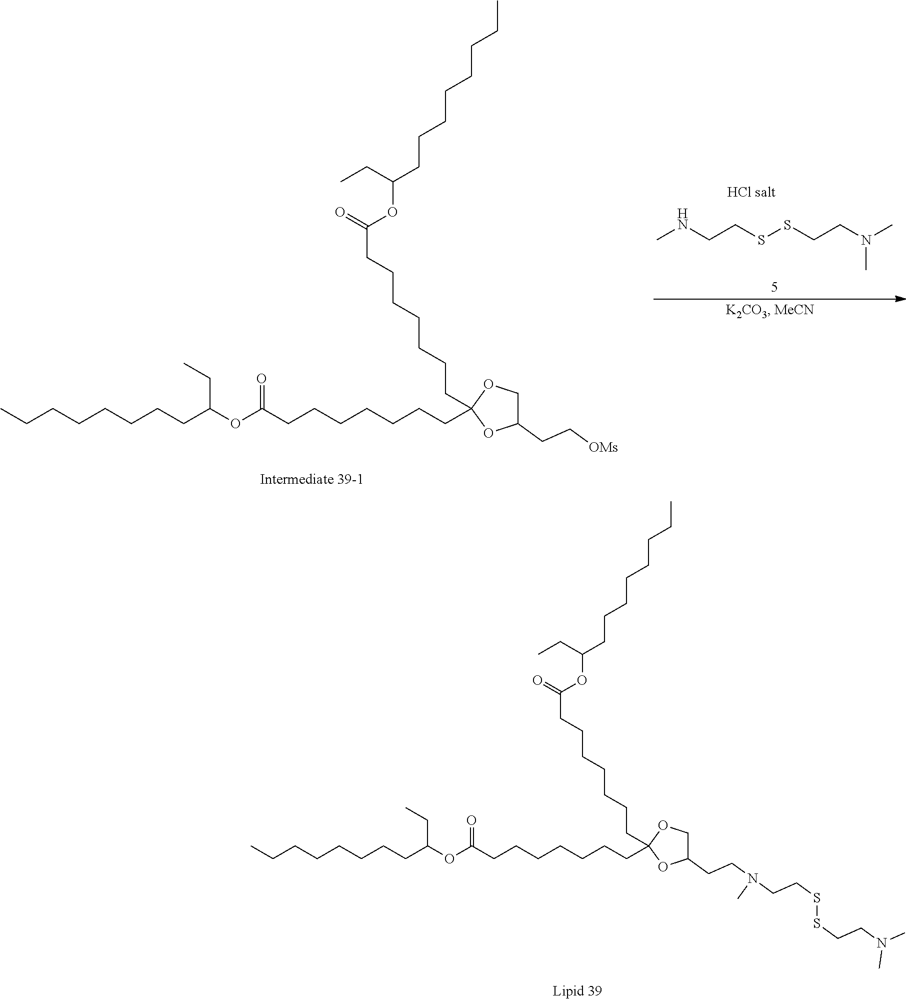

To a solution of compound 5 (131.61 mg, 570.20 μmol, 1.5 eq., HCl salt) in MeCN (4 mL) was added K₂CO₃ (157.62 mg, 1.14 mmol, 3 eq.) and intermediate 39-1 (300 mg, 380.13 μmol, 1 eq.). The mixture was stirred at 90° C. for 16 hours. The reaction mixture was filtered and the filtrate was concentrated under reduced pressure. The residue was purified by column chromatography (SiO₂, DCM/MeOH=1/0 to 10/1), followed by (column: PHS-Phenyl-Hexyl 150*25 mm*7 μm; mobile phase: [water (TFA)-MeOH]; gradient: 68%-98% B over 15 min) to give Lipid 39 (24 mg, 23.76% yield, 99% purity) as a yellow oil.

HRMS: m/z 887.6962 [M+1]⁺; ¹H NMR (400 MHz, CHLOROFORM-d) δ ppm 4.81 (quin, J=6.0 Hz, 2H), 4.13-4.02 (m, 2H), 3.51-3.43 (m, 1H), 2.86-2.78 (m, 4H), 2.74-2.61 (m, 4H), 2.57-2.41 (m, 2H), 2.31-2.25 (m, 12H), 2.15 (br s, 2H), 1.85-1.74 (m, 1H), 1.67-1.44 (m, 18H), 1.34-1.23 (m, 38H), 0.92-0.84 (m, 12H).

### Synthesis of Lipid 40. Lipid 41, and Lipid 42

### Procedure for Preparation of Compound 2

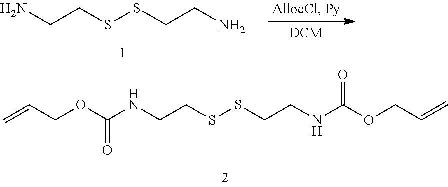 [SMILES: NCCSSCCN ; C=CCOC(=O)NCCSSCCNC(=O)OCC=C]

To a solution of compound 1 (5 g, 22.20 mmol, 1 eq., 2 HCl salts) in DCM (40 mL) was added pyridine (29.40 g, 371.68 mmol, 30 mL, 16.74 eq.) and AllocCl (10.70 g, 88.81 mmol, 9.42 mL, 4 eq.) at 0° C. The mixture was stirred at 20° C. for 4 hrs. The reaction mixture was quenched by addition of H₂O (10 mL) at 0° C., and then extracted with DCM (10 mL*2). The combined organic layers were dried over anhydrous Na₂SO₄, filtered and concentrated under reduced pressure. The residue was purified by column chromatography (SiO₂, Petroleum ether/Ethyl acetate=I/O to 3/1) to give compound 2 (2.1 g, 27.75% yield, 94% purity) as a white solid.

¹H NMR (400 MHz, CHLOROFORM-d) δ ppm 5.92 (tdd, J=5.6, 11.0, 16.8 Hz, 2H), 5.31 (br d, J=17.2 Hz, 4H), 5.22 (br d, J=10.4 Hz, 2H), 4.57 (br d, J=5.3 Hz, 4H), 3.51 (dd, J=18.4, 6.4 Hz, 4H), 2.82 (t, J=6.4 Hz, 4H).

### Procedure for Preparation of Compound 3

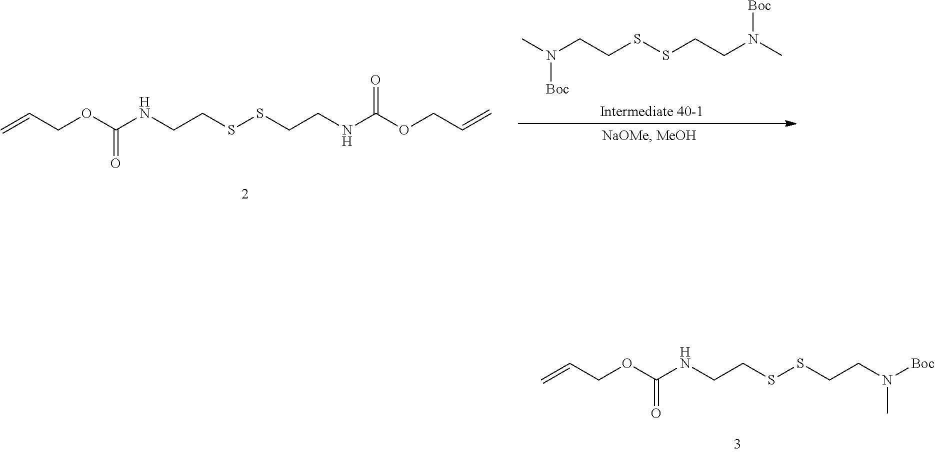

To a mixture of compound 2 (1.5 g, 4.68 mmol, 1 eq.) and intermediate 40-1 (1.78 g, 4.68 mmol, 1 eq.) in MeOH (5 mL) was added NaOMe (5.4 M MeOH solution, 7 mL, 8.07 eq.). The mixture was stirred at 25° C. for 12 hrs. The reaction mixture was quenched by addition of sat. NH₄Cl aqueous solution (5 mL) at 0° C., and then extracted with EtOAc (10 mL*2). The combined organic layers were washed with brine (10 mL), dried over anhydrous Na₂SO₄, filtered and concentrated under reduced pressure. The residue was purified by column chromatography (SiO₂, Petroleum ether/Ethyl acetate=I/O to 10/1 to 3/1) to give compound 3 (1 g, 56.07% yield, 92% purity) as a yellow oil.

¹H NMR (400 MHz, CHLOROFORM-d) δ ppm 5.97-5.86 (m, 1H), 5.72 (br s, 1H), 5.30 (dd, J=1.2, 17.2 Hz, 1H), 5.21 (br d, J=10.0 Hz, 1H), 4.56 (br d, J=5.4 Hz, 2H), 3.50 (br s, 4H), 2.89 (s, 3H), 2.81 (br d, J=6.0 Hz, 4H), 1.46 (s, 9H).

### Procedure for Preparation of Compound 4

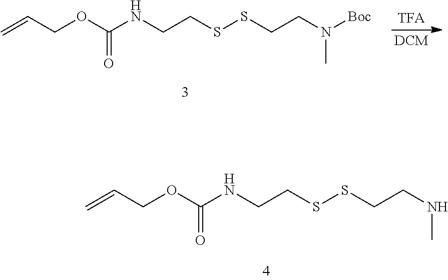 [SMILES: C=CCOC(=O)NCCSSCCN(C)C(=O)OC(C)(C)C ; C=CCOC(=O)NCCSSCCNC]

To a solution of compound 3 (1 g, 2.85 mmol, 1 eq.) in DCM (3 mL) was added TFA (4.39 g, 38.46 mmol, 2.86 mL, 13.48 eq.). The mixture was stirred at 20° C. for 2 hrs. The reaction mixture was concentrated under reduced pressure to give crude compound 4 (1.2 g, TFA salt) as a brown oil, which was used directly for next step without further purification.

¹H NMR (400 MHz, CHLOROFORM-d) δ ppm 10.87 (br s, 2H), 8.51 (br s, 1H), 5.90 (dt, J=5.2, 10.8 Hz, 1H), 5.35-5.21 (m, 2H), 4.58 (br s, 2H), 3.52 (br s, 2H), 3.41 (br s, 2H), 3.11-2.93 (m, 2H), 2.89-2.77 (m, 5H).

### Procedure for Preparation of Lipid 40

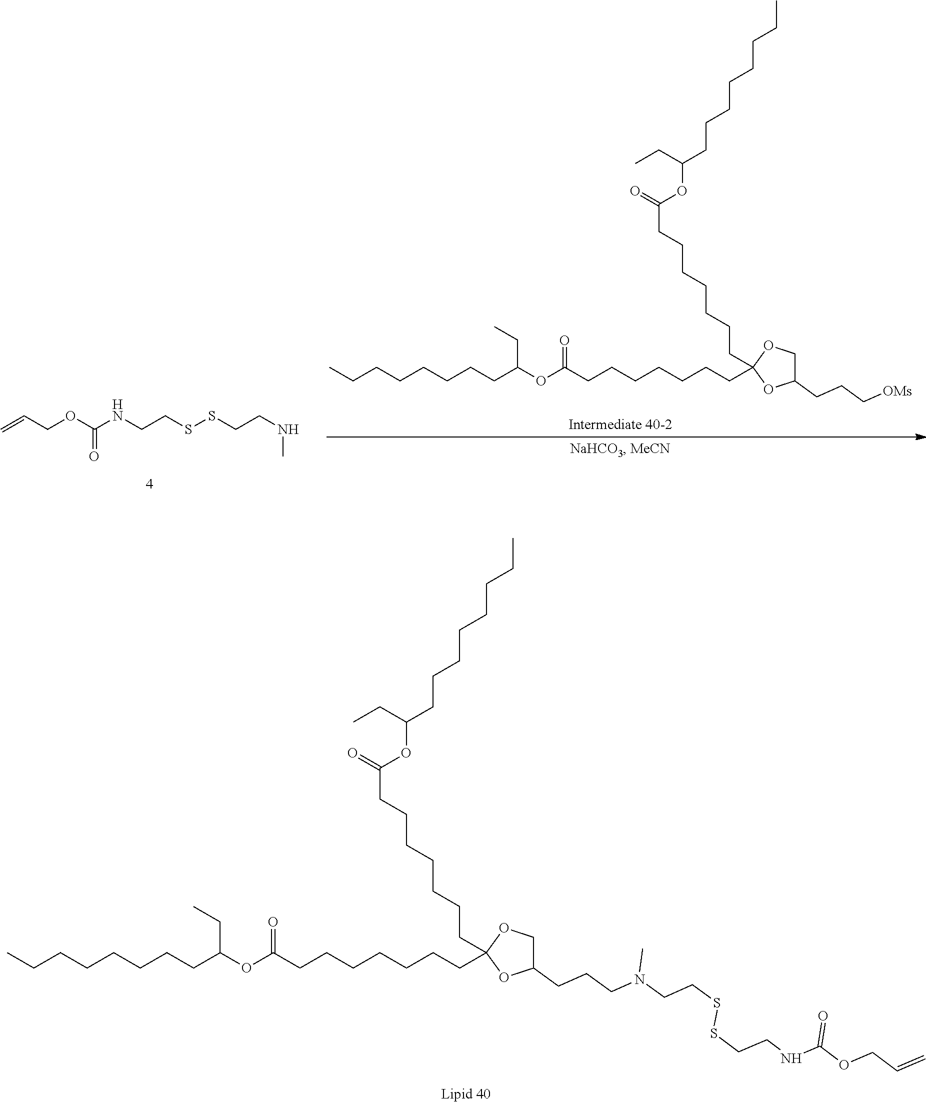 [SMILES: CCCCCCCCC(CC)OC(=O)CCCCCCCC1(CCCCCCCC(=O)OC(CC)CCCCCCCC)OCC(CCCOS(C)(=O)=O)O1 ; C=CCOC(=O)NCCSSCCNC ; C=CCOC(=O)NCCSSCCN(C)CCCC1COC(CCCCCCCC(=O)OC(CC)CCCCCCCC)(CCCCCCCC(=O)OC(CC)CCCCCCCC)O1]

To a mixture of intermediate 40-2 (1.2 g, 1.49 mmol, 1 eq.) and compound 4 (1.2 g, 3.29 mmol, 2.20 eq., TFA salt) in MeCN (15 mL) was added NaHCO₃ (753.03 mg, 8.96 mmol, 6 eq.). The mixture was stirred at 85° C. for 16 hrs. The reaction mixture was filtered and the filtrate was concentrated under reduced pressure. The residue was purified by column chromatography (SiO₂, PE/EA=10/1 to 5/1, 0.1% TEA as modifier), followed by another column purification (SiO₂, A/B=100/1 to 5/1, phase A: Petroleum ether:DCM=3:1, phase B: EtOAc, 0.1% TEA as modifier) to give Lipid 40 (650 mg, 43.17% yield, 95% purity) as a yellow oil.

HRMS: m/z 957.7033 [M+1]⁺; ¹H NMR (400 MHz, CHLOROFORM-d) δ ppm 6.00-5.86 (m, 1H), 5.32 (dd, J=1.3, 17.2 Hz, 1H), 5.23 (br d, J=10.4 Hz, 1H), 4.82 (t, J=6.0 Hz, 2H), 4.58 (br d, J=5.2 Hz, 2H), 4.12-4.03 (m, 2H), 3.74-3.64 (m, 2H), 3.52 (q, J=6.4 Hz, 2H), 3.49-3.44 (m, 1H), 2.83 (t, J=6.4 Hz, 2H), 2.29 (s, 4H), 1.74-1.46 (m, 22H), 1.40-1.19 (m, 42H), 0.93-0.83 (m, 12H).

### Procedure for Preparation of Lipid 41

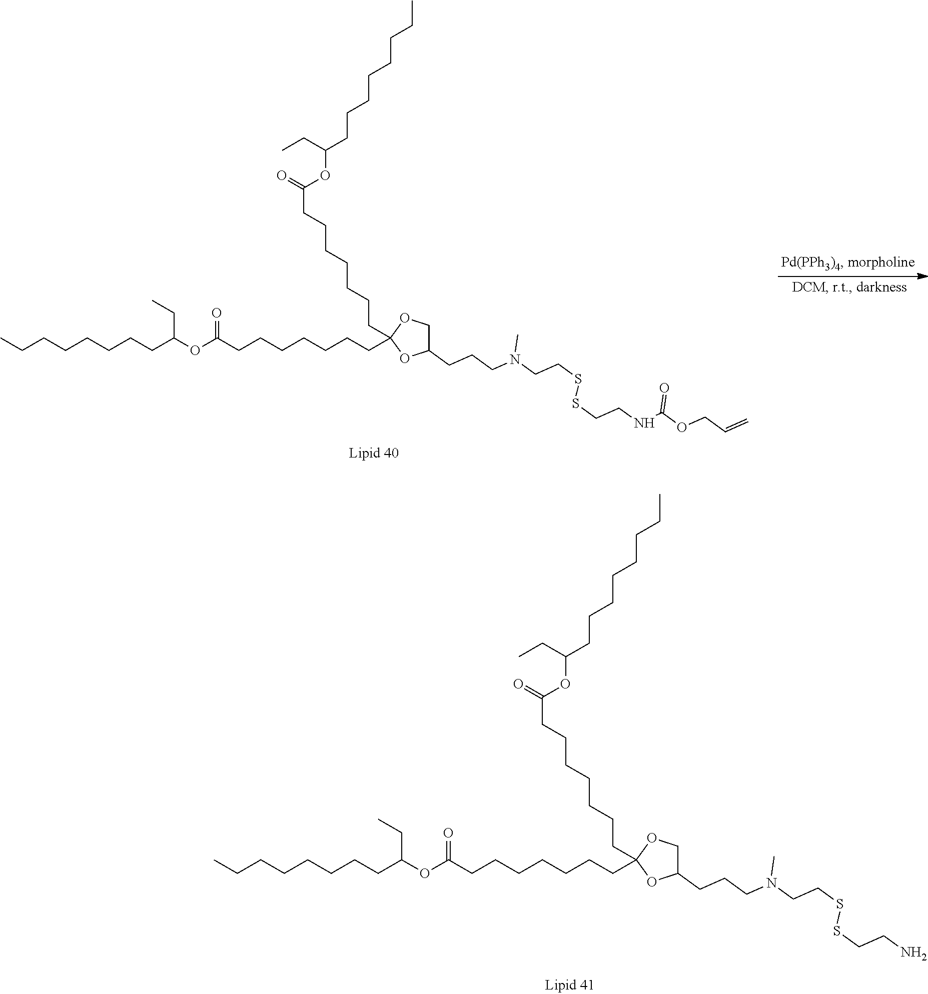

To a solution of Lipid 40 (410 mg, 428.20 μmol, 1 eq.) in DCM (3 mL) was added Pd (PPh₃)₄ (49.48 mg, 42.82 μmol, 0.1 eq.) and morpholine (18.65 mg, 214.10 μmol, 18.84 μL, 0.5 eq.). The mixture was stirred at 25° C. for 2 hrs. The reaction mixture was quenched by addition of H₂O (5 mL), and then extracted with DCM (5 mL*2). The combined organic layers were filtered and the filtrate was concentrated under reduced pressure. The residue was purified by prep-HPLC (column: X-Select CSH Phenyl-Hexyl 100*30 5u; mobile phase: [H₂O (0.1% TFA)-EtOH]; gradient: 58%-88% B over 15.0 min) to give Lipid 41 (160 mg, 22.69% yield, 95% purity) as a colourless oil.

HRMS: m/z 873.6771 [M+1]⁺; ¹H NMR (400 MHz, CHLOROFORM-d) δ ppm 4.81 (s, 2H), 4.04 (s, 2H), 3.45 (s, 1H), 3.03 (t, J=6.4 Hz, 2H), 2.85-2.75 (m, 4H), 2.72 (br d, J=8.0 Hz, 2H), 2.43 (s, 2H), 2.32-2.24 (m, 7H), 1.90 (br s, 2H), 1.68-1.47 (m, 20H), 1.38-1.21 (m, 40H), 0.92-0.82 (m, 12H).

### Procedure for Preparation of Lipid 42

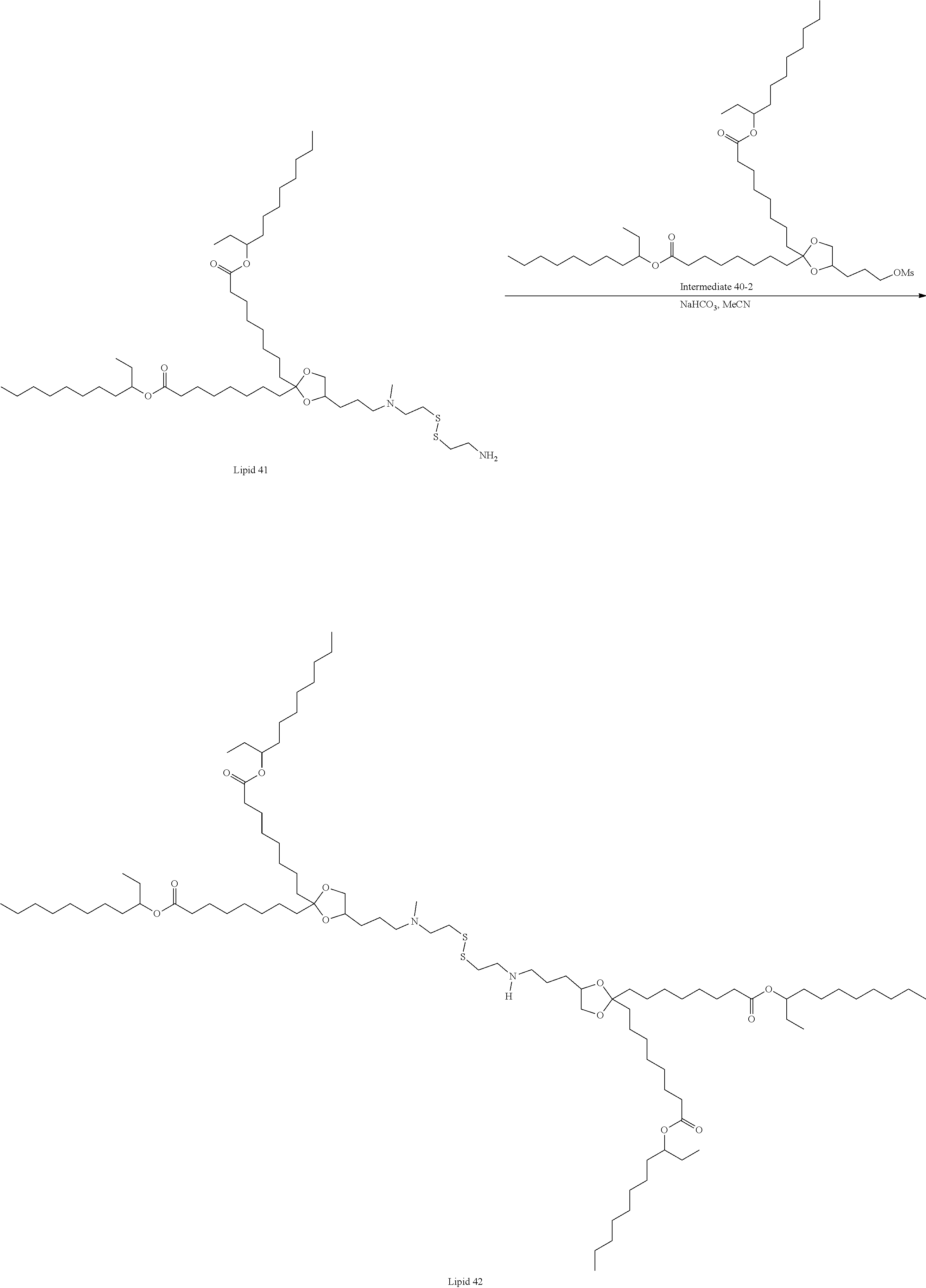

To a mixture of Lipid 41 (120 mg, 137.39 μmol, 1 eq.) and intermediate 40-2 (275.89 mg, 343.48 μmol, 2.5 eq.) in MeCN (4 mL) was added NaHCO₃ (34.63 mg, 412.17 μmol, 3 eq.). The mixture was stirred at 85° C. for 16 hrs. The reaction mixture was filtered and the filtrate was concentrated under reduced pressure. The residue was purified by prep-HPLC (column: X-Select CSH Phenyl-Hexyl 100*30 5u; mobile phase: [H₂O (0.1% TFA)-EtOH]; gradient: 68%-98% B over 17.0 min), followed by prep-SFC (column: DAICEL CHIRALPAK IF (250 mm*30 mm, 10 μm); mobile phase: [CO₂-IPA:ACN=4:1 (0.1% NH₃H₂O)]; B %: 45%, isocratic elution mode), and then purified by prep-HPLC (column: X-Select CSH Phenyl-Hexyl 100*30 5u; mobile phase: [H₂O (0.225% FA)-EtOH]; gradient: 68%-98% B over 15.0 min) to give Lipid 42 (20 mg, 66.67% yield) as a colourless oil.

HRMS: m/z 1581.2922 [M+1]⁺; ¹H NMR (400 MHz, CHLOROFORM-d) δ ppm 4.82 (t, J=6.0 Hz, 4H), 4.04 (br d, J=4.4 Hz, 4H), 3.50-3.40 (m, 2H), 3.02 (br s, 2H), 2.96-2.82 (m, 4H), 2.76 (br d, J=5.2 Hz, 4H), 2.55-2.45 (m, 2H), 2.29 (br t, J=7.6 Hz, 11H), 1.73-1.44 (m, 42H), 1.38-1.22 (m, 80H), 0.94-0.82 (m, 24H).

### Synthesis of Lipid 43 and Lipid 44

### Procedure for Preparation of Lipid 43 and Lipid 44

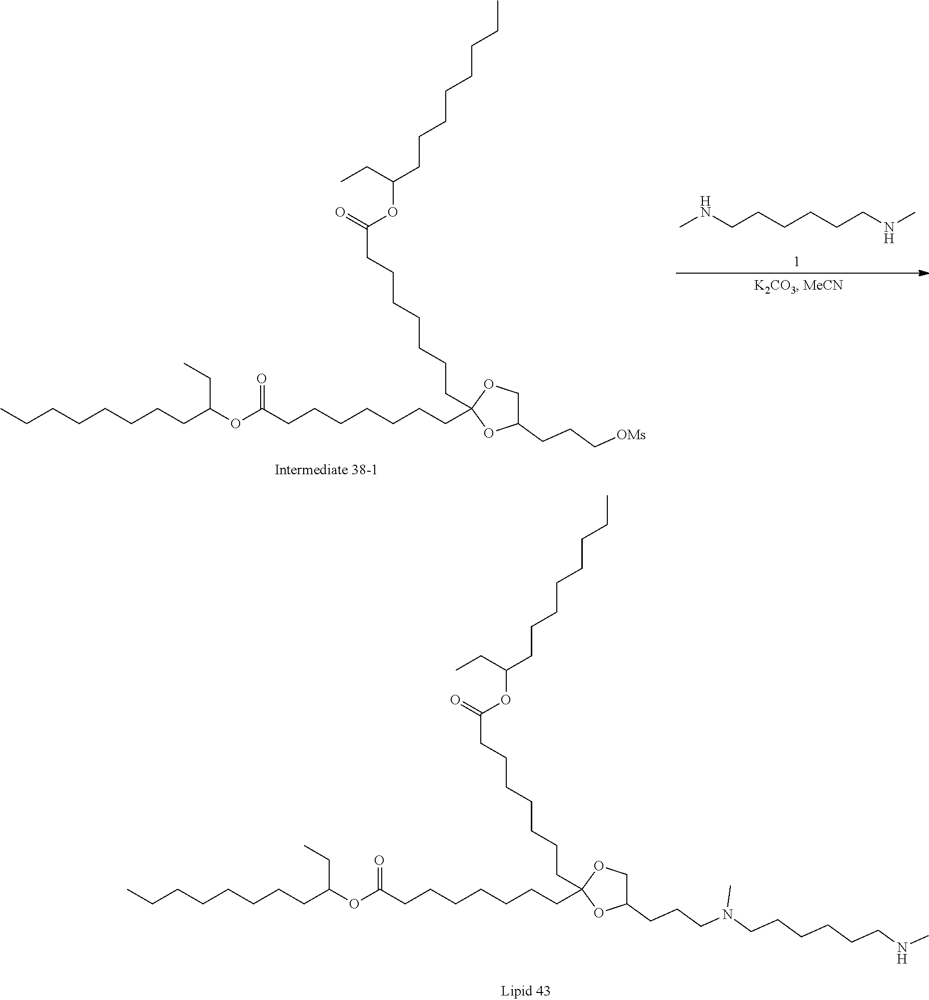

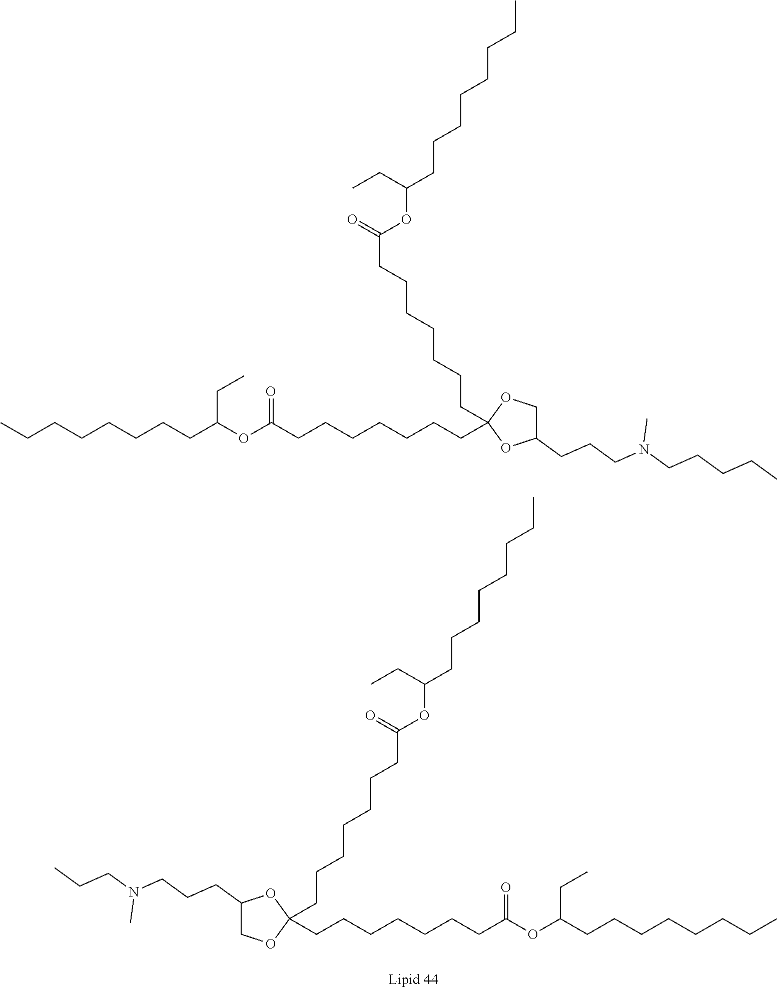

To a mixture of intermediate 38-1 (500 mg, 622.49 μmol, 1 eq.) and compound 1 (269.40 mg, 1.87 mmol, 3 eq.) in MeCN (6 mL) was added K₂CO₃ (258.10 mg, 1.87 mmol, 3 eq.), and then the mixture was stirred at 85° C. for 16 hrs. The reaction mixture was filtered and the filtrate was concentrated under reduced pressure. The residue was purified by prep-HPLC (column: UniSil 3-100 C18 Ultra (150*25 mm*3 μm); mobile phase: [H₂O (0.225% FA)-ACN]; gradient: 35%-65% B over 14.0 min) to give crude Lipid 43 (300 mg) as a colourless oil and crude Lipid 44 (100 mg) as a colourless oil.

The crude Lipid 43 (300 mg) was further purified by prep-HPLC (column: X-Select CSH Phenyl-Hexyl 100*30 5u; mobile phase: [H₂O (0.1% TFA)-MeOH]; gradient: 68%-98% B over 15.0 min) to give Lipid 43 (245 mg, 46.23% yield) as a colourless oil.

HRMS: m/z 851.7820 [M+1]⁺; ¹H NMR (400 MHz, CHLOROFORM-d) d ppm 4.87-4.75 (m, 2H), 4.11-4.00 (m, 2H), 3.48-3.42 (m, 1H), 2.94-2.83 (m, 2H), 2.74-2.65 (m, 4H), 2.63 (s, 3H), 2.50-2.45 (m, 3H), 2.29 (s, 4H), 1.44 (br d, J=8.0 Hz, 1H), 1.41-1.21 (m, 42H), 0.97-0.80 (m, 12H).

The crude Lipid 44 (100 mg) was further purified by prep-HPLC (column: X-Select CSH Phenyl-Hexyl 100*30 5u; mobile phase: [H₂O (0.1% TFA)-MeOH]; gradient: 80%-98% B over 15.0 min) to give Lipid 44 (42 mg, 4.33% yield) as a colourless oil.

HRMS: m/z 1559.3945 [M+1]⁺; ¹H NMR (400 MHz, CHLOROFORM-d) δ ppm 4.82 (t, J=6.2 Hz, 4H), 4.15-3.97 (m, 4H), 3.51-3.37 (m, 2H), 2.73-2.16 (m, 21H), 1.56 (br dd, J=7.2, 13.5 Hz, 44H), 1.39-1.21 (m, 84H), 0.95-0.81 (m, 24H).

### Synthesis of Lipid 45 and Lipid 46

### Procedure for Preparation of Lipid 45 and Lipid 46

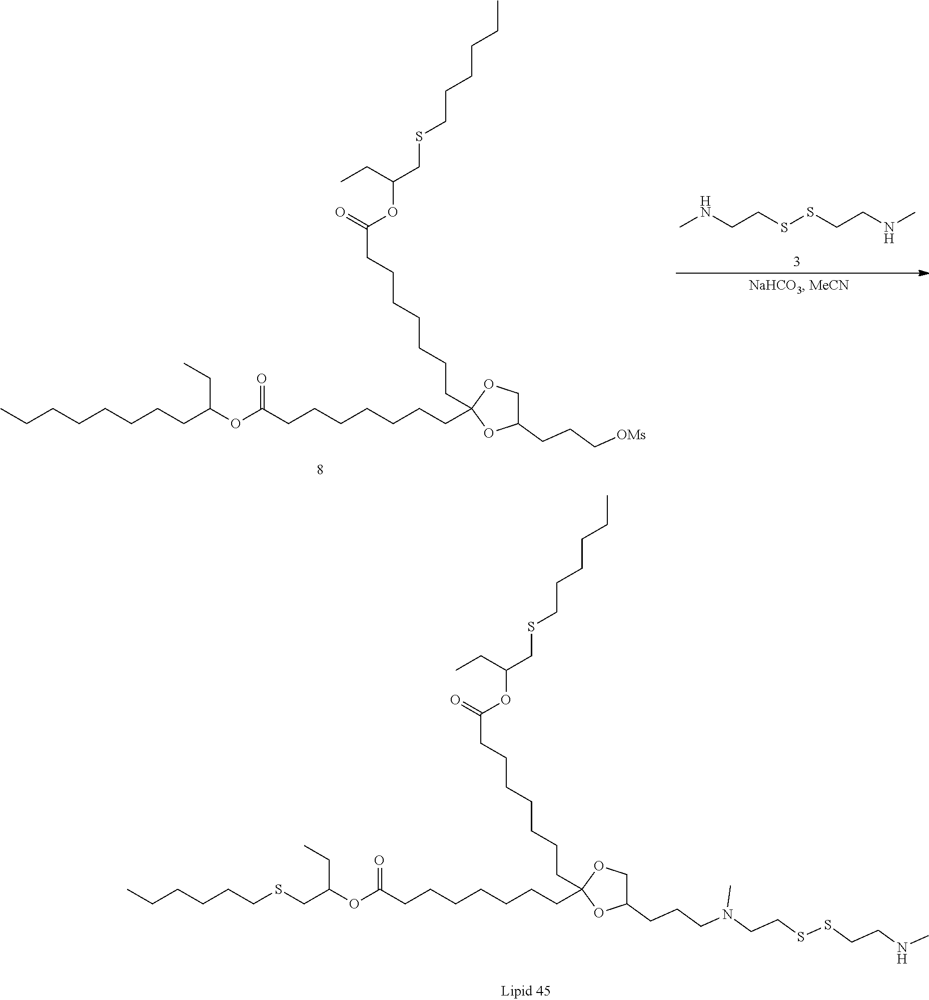

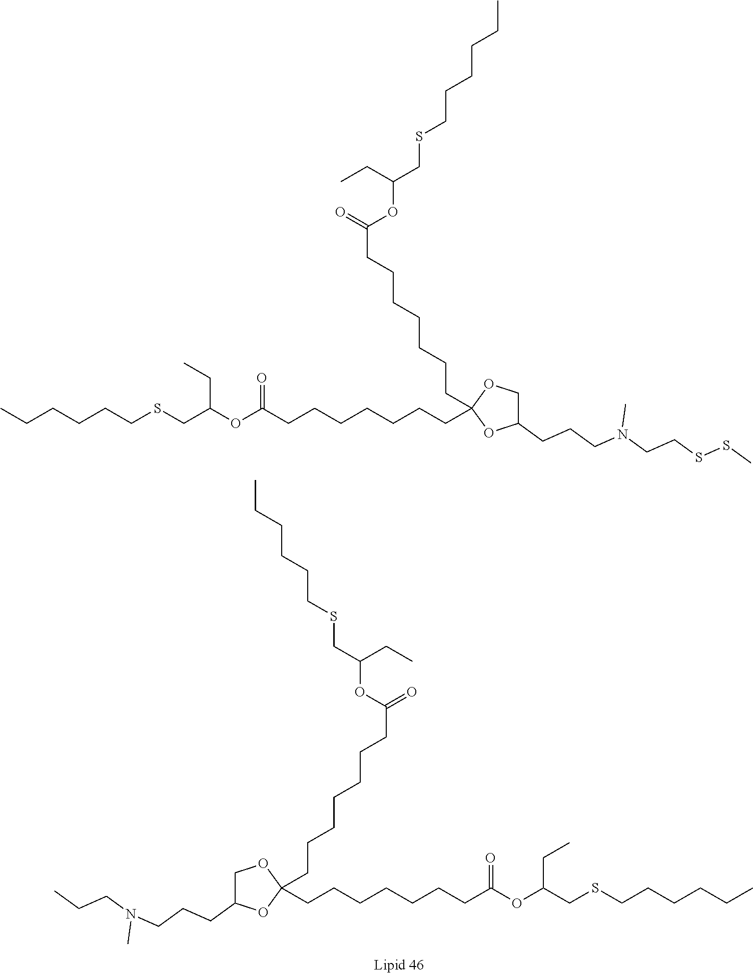

To a mixture of compound 8 (400 mg, 476.59 μmol, 1 eq.) and NaHCO₃ (120.11 mg, 1.43 mmol, 3 eq.) in MeCN (5 mL) was added compound 3 (362.10 mg, 1.43 mmol, 3 eq., 2 HCl salt) at 25° C., and then the mixture was stirred at 85° C. for 16 hrs. The reaction mixture was filtered and the filtrate was concentrated under reduced pressure. The residue was purified by prep-HPLC (column: X-Select CSH Phenyl-Hexyl 100*30 5u; mobile phase: [H₂O (0.1% TFA)-MeOH]; gradient: 62%-92% B over 14.0 min), followed by prep-HPLC (column: X-Select CSH Phenyl-Hexyl 100*30 5u; mobile phase: [H₂O (0.1% TFA)-MeOH]; gradient: 65%-95% B over 15.0 min) and prep-HPLC (column: X-Select CSH Phenyl-Hexyl 100*30 5u; mobile phase: [H₂O (0.1% TFA)-MeOH]; gradient: 80%-98% B over 15.0 min) respectively to give Lipid 45 (139 mg, 31.59% yield) as a yellow oil and Lipid 46 (20 mg, 2.52% yield) as a yellow oil.

Lipid 45: HRMS: m/z 923.6070 [M+1]⁺; ¹H NMR (400 MHz, CHLOROFORM-d) δ ppm 4.96-4.85 (m, 2H), 4.10-3.99 (m, 2H), 3.49-3.40 (m, 1H), 3.04-2.98 (m, 2H), 2.92-2.81 (m, 4H), 2.77-2.71 (m, 2H), 2.65 (d, J=6.4 Hz, 4H), 2.53 (s, 7H), 2.46 (br t, J=6.8 Hz, 3H), 2.30 (s, 7H), 1.77 (ddd, J=4.8, 7.4, 14.1 Hz, 2H), 1.68-1.48 (m, 18H), 1.40-1.25 (m, 28H), 0.95-0.85 (m, 12H).

Lipid 46: HRMS: m/z 1667.1321 [M+1]⁺; ¹H NMR (400 MHz, CHLOROFORM-d) δ ppm 4.97-4.85 (m, 4H), 4.04 (br d, J=5.6 Hz, 4H), 3.48-3.41 (m, 2H), 3.02-2.83 (m, 7H), 2.65 (d, J=6.0 Hz, 10H), 2.54 (t, J=7.2 Hz, 10H), 2.49-2.40 (m, 4H), 2.31 (t, J=7.6 Hz, 8H), 1.82-1.70 (m, 6H), 1.69-1.52 (m, 34H), 1.45-1.21 (m, 59H), 0.90 (td, J=7.2, 9.7 Hz, 24H).

### Synthesis of Lipid 47 and Lipid 48

### Procedure for Preparation of Compound 2

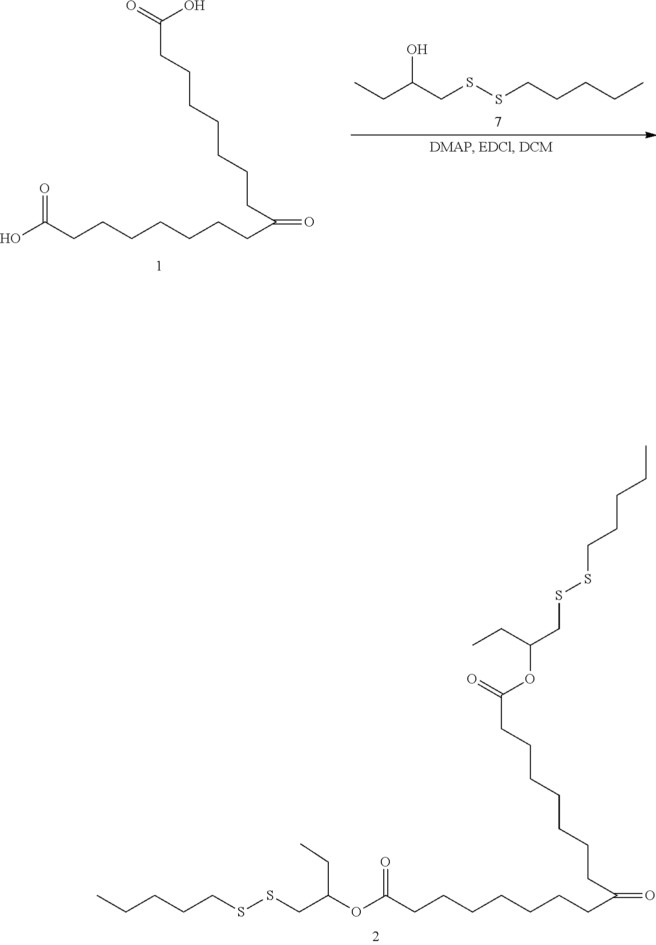 [SMILES: O=C(O)CCCCCCCC(=O)CCCCCCCC(=O)O ; CCCCCSSCC(O)CC ; CCCCCSSCC(CC)OC(=O)CCCCCCCC(=O)CCCCCCCC(=O)OC(CC)CSSCCCCC]

To a mixture of compound 1 (500 mg, 1.59 mmol, 1 eq.) and compound 7 (662.76 mg, 3.18 mmol, 2 eq.) in DCM (10 mL) was added EDCI (1.52 g, 7.95 mmol, 5 eq.) and DMAP (582.83 mg, 4.77 mmol, 3 eq.), and then the mixture was stirred at 25° C. for 12 hrs. The reaction mixture was poured into aq. NH₄Cl (sat., 20 mL) and extracted with DCM (20 mL*3). The combined organic layers were dried over anhydrous Na₂SO₄, filtered and concentrated under reduced pressure. The residue was purified by column chromatography (SiO₂, Petroleum ether/Ethyl acetate=1/to 30/1) to give compound 2 (1.08 g, 84.3% yield, 86.3% purity) as a yellow oil.

¹H NMR (400 MHz, CHLOROFORM-d) δ ppm 5.04 (quin, J=6.0 Hz, 2H), 2.87 (d, J=6.0 Hz, 4H), 2.76-2.67 (m, 4H), 2.38 (t, J=7.2 Hz, 4H), 2.32 (t, J=7.6 Hz, 4H), 1.77-1.54 (m, 16H), 1.41-1.26 (m, 20H), 0.96-0.88 (m, 12H).

### Procedure for Preparation of Compound 4

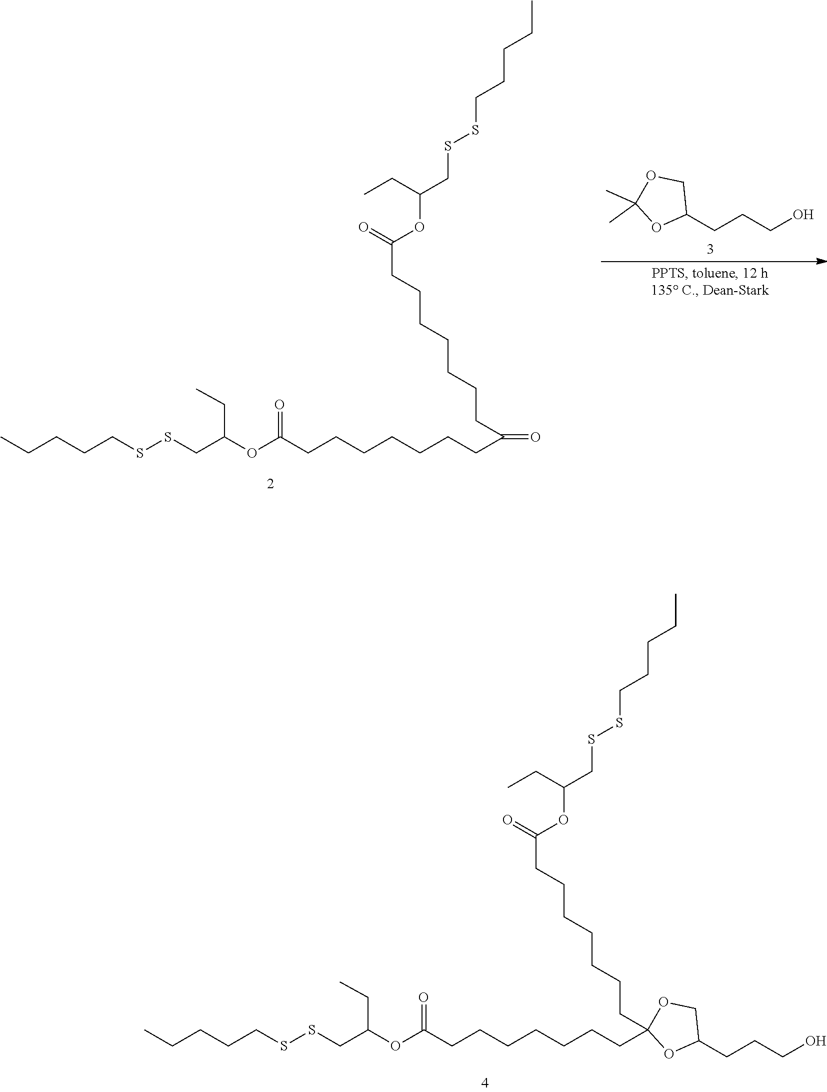 [SMILES: CCCCCSSCC(CC)OC(=O)CCCCCCCC(=O)CCCCCCCC(=O)OC(CC)CSSCCCCC ; CC1(C)OCC(CCCO)O1 ; CCCCCSSCC(CC)OC(=O)CCCCCCCC1(CCCCCCCC(=O)OC(CC)CSSCCCCC)OCC(CCCO)O1]

To a mixture of compound 2 (1.08 g, 1.55 mmol, 1 eq.) and compound 3 (622.26 mg, 3.88 mmol, 2.5 eq.) in toluene (20 mL) was added PPTS (195.21 mg, 776.81 μmol, 0.5 eq.), and then the mixture was stirred at 135° C. for 12 hrs equipped with Dean-Stark. The reaction mixture was quenched by addition of sat. NaHCO₃ aqueous solution (20 mL) at 25° C., and then diluted with H₂O (20 mL) and extracted with EtOAc (30 mL*2). The combined organic layers were washed with brine (10 mL*2), dried over anhydrous Na₂SO₄, filtered and concentrated under reduced pressure. The residue was purified by column chromatography (SiO₂, Petroleum ether/Ethyl acetate=10/1˜5/1˜1/3) to give compound 4 (900 mg, 67.57% yield, 93% purity) as a colourless oil.

¹H NMR (400 MHz, CHLOROFORM-d) δ ppm 5.10-4.99 (m, 2H), 4.18-4.00 (m, 2H), 3.76-3.63 (m, 2H), 3.52-3.43 (m, 1H), 2.94-2.84 (m, 4H), 2.76-2.66 (m, 4H), 2.37-2.27 (m, 4H), 1.79-1.62 (m, 20H), 1.43-1.26 (m, 26H), 0.93 (s, 12H).

### Procedure for Preparation of Compound 5

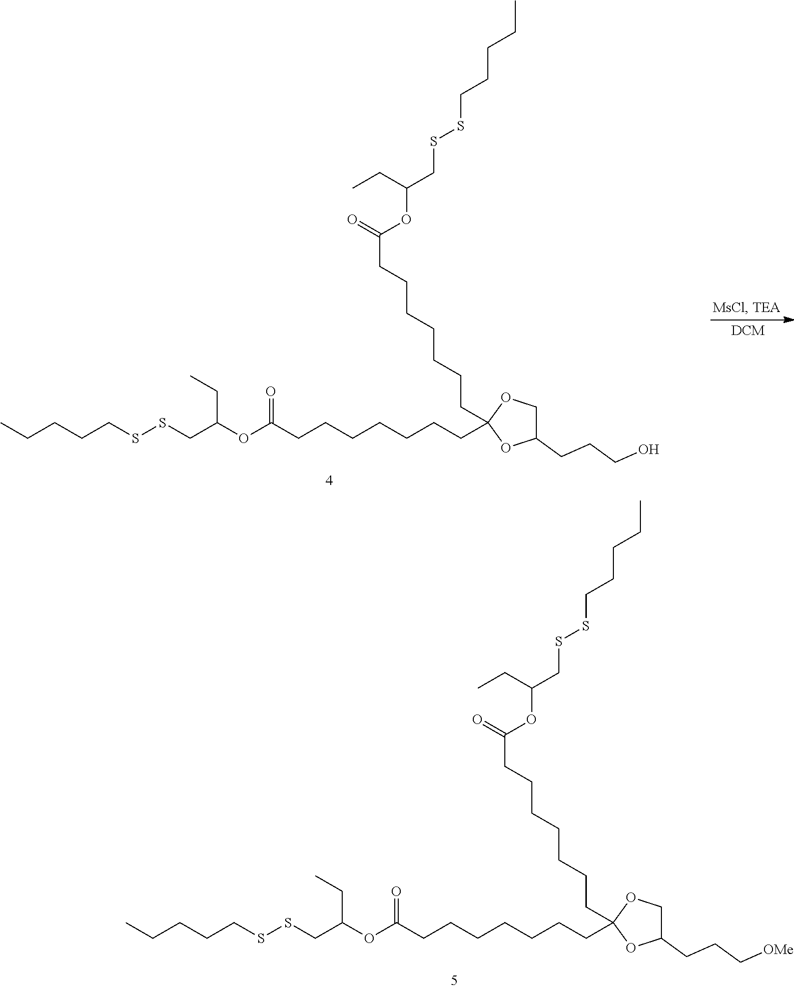 [SMILES: CCCCCSSCC(CC)OC(=O)CCCCCCCC1(CCCCCCCC(=O)OC(CC)CSSCCCCC)OCC(CCCO)O1 ; CCCCCSSCC(CC)OC(=O)CCCCCCCC1(CCCCCCCC(=O)OC(CC)CSSCCCCC)OCC(CCCOC)O1]

To a solution of compound 4 (800 mg, 1.00 mmol, 1 eq.) in DCM (10 mL) was added MsCl (344.82 mg, 3.01 mmol, 3 eq.) and TEA (507.67 mg, 5.02 mmol, 5 eq.) at 0° C., and then the reaction mixture was stirred at 20° C. for 2 hrs. The reaction mixture was poured into ice-water (10 mL), and then extracted with DCM (10 mL*3). The combined organic layers were concentrated under reduced pressure. The residue was purified by column chromatography (SiO₂, PE/EA=10/1 to 5/1, eluent with 0.1% TEA) to give compound 5 (650 mg, 74.00% yield) as a colourless oil.

¹H NMR (400 MHz, CHLOROFORM-d) δ ppm 5.09-4.98 (m, 2H), 4.34-4.22 (m, 2H), 4.11-4.01 (m, 2H), 3.52-3.42 (m, 1H), 3.02 (s, 3H), 2.87 (d, J=6.0 Hz, 4H), 2.71 (dt, J=1.2, 7.3 Hz, 4H), 2.32 (t, J=7.6 Hz, 4H), 1.97-1.56 (m, 20H), 1.42-1.26 (m, 24H), 0.96-0.87 (m, 12H).

### Procedure for Preparation of Lipid 47 and Lipid 48

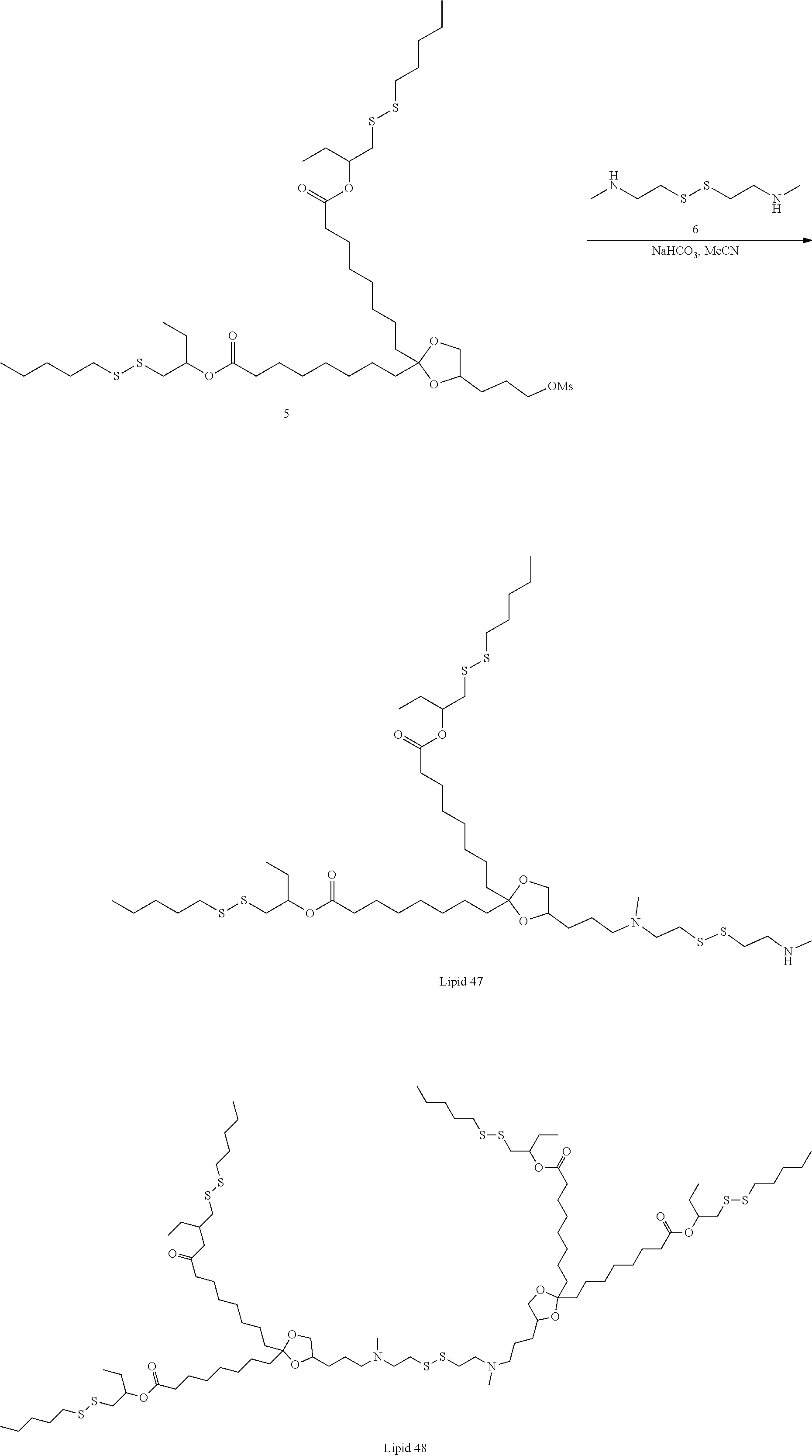

To a mixture of compound 5 (500 mg, 571.18 μmol, 1 eq.) and compound 6 (433.97 mg, 1.71 mmol, 3 eq., 2 HCl salt) in MeCN (5 mL) was added NaHCO₃ (143.96 mg, 1.71 mmol, 3 eq.), and then the mixture was stirred at 85° C. for 16 hrs. The reaction mixture was filtered and the filtrate was concentrated under reduced pressure. The residue was purified by prep-HPLC (column: X-Select CSH Phenyl-Hexyl 100*30 5u; mobile phase: [H₂O (0.1% TFA)-MeOH]; gradient: 68%-98% B over 15.0 min) to give crude Lipid 47 and crude Lipid 48.

The crude Lipid 47 was further purified by prep-HPLC (column: X-Select CSH Phenyl-Hexyl 100*30 5u; mobile phase: [H₂O) (0.1% TFA)-EtOH]; gradient: 58%-88% B over 15.0 min) to give Lipid 47 (54 mg, 9.85% yield) as a yellow oil.

HRMS: m/z 959.5209 [M+1]⁺; ¹H NMR (400 MHz, CHLOROFORM-d) δ ppm 5.03 (td, J=6.0, 12.3 Hz, 2H), 4.97-4.76 (m, 2H), 4.11-3.94 (m, 2H), 3.49-3.40 (m, 1H), 3.11 (s, 2H), 2.93 (s, 2H), 2.87 (d, J=6.0 Hz, 6H), 2.79-2.66 (m, 6H), 2.60 (s, 3H), 2.49 (br t, J=7.2 Hz, 2H), 2.36-2.26 (m, 7H), 1.82-1.48 (m, 20H), 1.43-1.24 (m, 24H), 0.96-0.86 (m, 12H).

The crude Lipid 48 was further purified by prep-HPLC (column: X-Select CSH Phenyl-Hexyl 100*30 5u; mobile phase: [H₂O) (0.1% TFA)-MeOH]; gradient: 68%-98% B over 15.0 min) and prep-HPLC (column: X-Select CSH Phenyl-Hexyl 100*30 5u; mobile phase: [H₂O (0.1% TFA)-EtOH]; gradient: 68%-98% B over 15.0 min) to give Lipid 48 (25 mg, 2.52% yield) as a colourless oil.

HRMS: m/z 1737.9442 [M+1]⁺; ¹H NMR (400 MHz, CHLOROFORM-d) δ ppm 5.04 (quin, J=6.0 Hz, 4H), 4.11-4.01 (m, 4H), 3.48-3.42 (m, 2H), 3.14 (q, J=7.2 Hz, 4H), 3.09-3.00 (m, 4H), 2.87 (d, J=6.1 Hz, 9H), 2.71 (t, J=7.2 Hz, 12H), 2.32 (t, J=7.6 Hz, 8H), 1.83-1.49 (m, 40H), 1.43-1.24 (m, 53H), 0.92 (q, J=6.8 Hz, 24H).

### Synthesis of Lipid 49

### Procedure for Preparation of Compound 3

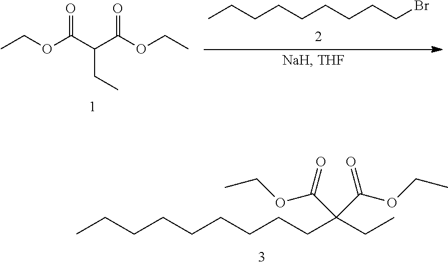

To a mixture of compound 1 (5 g, 26.56 mmol, 4.98 mL, 1 eq.) and compound 2 (8.25 g, 39.85 mmol, 1.5 eq.) in THF (50 mL) was added NaH (1.59 g, 39.85 mmol, 60% purity, 1.5 eq.) at 25° C., and then the mixture was stirred at 60° C. for 2 hrs. The reaction mixture was poured into water (200 mL) and extracted with EtOAc (200 mL). The organic layer was washed with brine (100 mL), dried over anhydrous Na₂SO₄, filtered and concentrated in vacuo. The residue was purified by column chromatography (SiO₂, Petroleum ether/Ethyl acetate=20/1 to 10/1) to give compound 3 (8 g, 95.77% yield) as a white solid.

¹H NMR (400 MHz, CHLOROFORM-d) δ ppm 4.18 (q, J=7.2 Hz, 4H), 2.01-1.79 (m, 4H), 1.34-1.21 (m, 18H), 1.18-1.09 (m, 2H), 0.91-0.86 (m, 3H), 0.82 (t, J=7.6 Hz, 3H).

### Procedure for Preparation of Compound 4

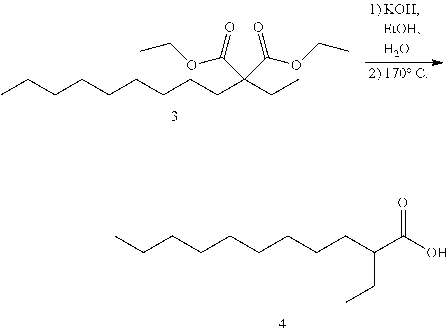

To a solution of compound 3 (5 g, 15.90 mmol, 1 eq.) in EtOH (50 mL) was added KOH (2 M aqueous solution, 55.65 mL, 7 eq.) at 0° C., and then the resulting mixture was stirred at 80° C. for 12 hrs. The reaction mixture was poured into water (200 mL) and extracted with EtOAc (200 mL). The organic layer was washed with brine (100 mL), dried over anhydrous Na₂SO₄, filtered and concentrated in vacuo. And then the obtained white oil (3.7 g) was directly heated to 170° C. for 4 hrs under stirring. The residue was purified by column chromatography (SiO₂, Petroleum ether/Ethyl acetate=20/1 to 10/1) to give compound 4 (1.8 g, 72.32% yield) as a white oil.

¹H NMR (400 MHz, CHLOROFORM-d) δ ppm 2.30 (tt, J=5.6, 8.5 Hz, 1H), 1.73-1.41 (m, 4H), 1.39-1.18 (m, 14H), 1.01-0.83 (m, 6H).

### Procedure for Preparation of Compound 7

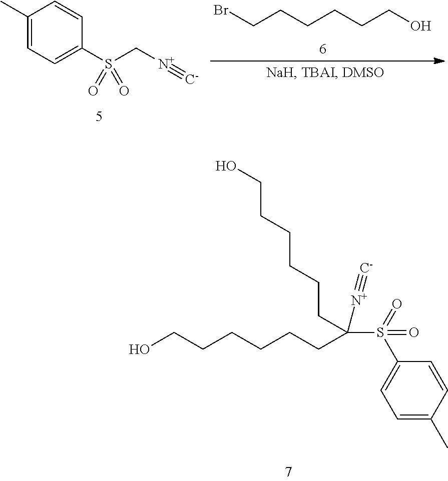

To DMSO (50 mL) was added NaH (2.56 g, 64.02 mmol, 60% purity, 2.5 eq.) in portions at 10° C., and then the mixture was stirred at 25° C. for 1 hr. Compound 5 (5 g, 25.61 mmol, 1 eq.) was added in portions at the same temperature, followed by TBAI (945.94 mg, 2.56 mmol, 0.1 eq.). After stirring at 25° C. for another 15 mins, compound 6 (9.74 g, 53.78 mmol, 2.1 eq.) was added portionwise, and then the mixture was stirred at 25° C. for 1 hrs under N₂ atmosphere. The reaction mixture was poured into water (200 mL) and extracted with EtOAc (200 mL). The organic layer was washed with brine (120 mL), dried over anhydrous Na₂SO₄, filtered and concentrated in vacuo to give crude compound 7 (12 g) as a white oil, which was used directly for next step without further purification.

LCMS: m/z 396.2 [M+1]⁺; ¹H NMR (400 MHz, CHLOROFORM-d) 0 ppm 7.92-7.80 (m, 2H), 7.47-7.35 (m, 2H), 3.71-3.52 (m, 4H), 3.48-3.34 (m, 1H), 2.53-2.41 (m, 3H), 2.08-1.83 (m, 5H), 1.42-1.18 (m, 23H), 0.95-0.74 (m, 7H).

### Procedure for Preparation of Compound 8

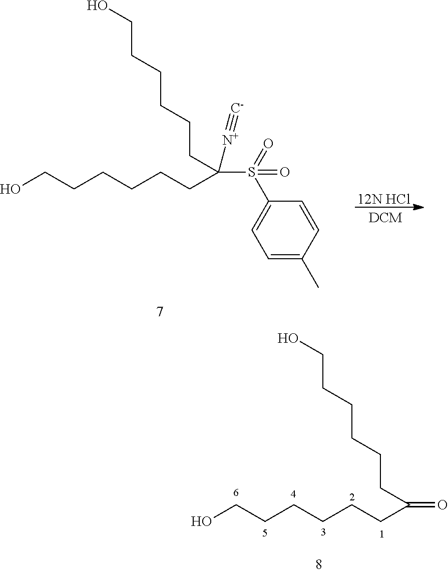

To a solution of compound 7 (3 g, 7.58 mmol, 1 eq.) in DCM (45 mL) was added conc. HCl (15 mL) at 25° C., and then the mixture was stirred at 25° C. for 2 hrs. The reaction mixture was directly freeze-dried and the residue was purified by column chromatography (SiO₂, Petroleum ether/Ethyl acetate=10/1 to 1/1) to give compound 8 (560 mg, 13.46% yield) as a white solid.

¹H NMR (400 MHz, CHLOROFORM-d) δ ppm 3.64 (t, J=6.6 Hz, 4H), 2.40 (t, J=7.2 Hz, 4H), 1.63-1.53 (m, 10H), 1.42-1.28 (m, 8H).

### Procedure for Preparation of Compound 9

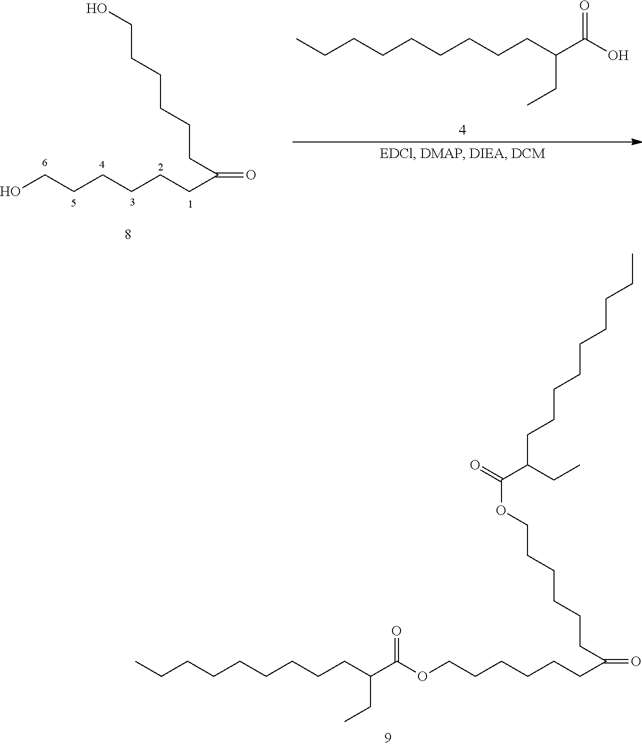 [SMILES: O=C(CCCCCCO)CCCCCCO ; CCCCCCCCCC(CC)C(=O)O ; CCCCCCCCCC(CC)C(=O)OCCCCCCC(=O)CCCCCCOC(=O)C(CC)CCCCCCCCC]

To a mixture of compound 8 (560 mg, 2.43 mmol, 1 eq.) and compound 4 (1.04 g, 4.86 mmol, 2 eq.) in DCM (5 mL) was added DIEA (2.83 g, 21.88 mmol, 3.81 mL, 9 eq.), EDCI (1.86 g, 9.72 mmol, 4 eq.) and DMAP (297.01 mg, 2.43 mmol, 1 eq.), and then the mixture was stirred at 40° C. for 12 hrs. The reaction mixture was concentrated under reduced pressure and the residue was purified by column chromatography (SiO₂, Petroleum ether/Ethyl acetate=20/1 to 10/1) to give compound 9 (1.3 g, 83.26% yield) as a yellow oil.

¹H NMR (400 MHz, CHLOROFORM-d) δ ppm 4.07 (t, J=6.6 Hz, 4H), 2.39 (t, J=7.4 Hz, 4H), 2.25 (tt, J=5.4, 8.8 Hz, 2H), 1.69-1.51 (m, 18H), 1.26 (s, 34H), 0.94-0.84 (m, 12H).

### Procedure for Preparation of Compound 11

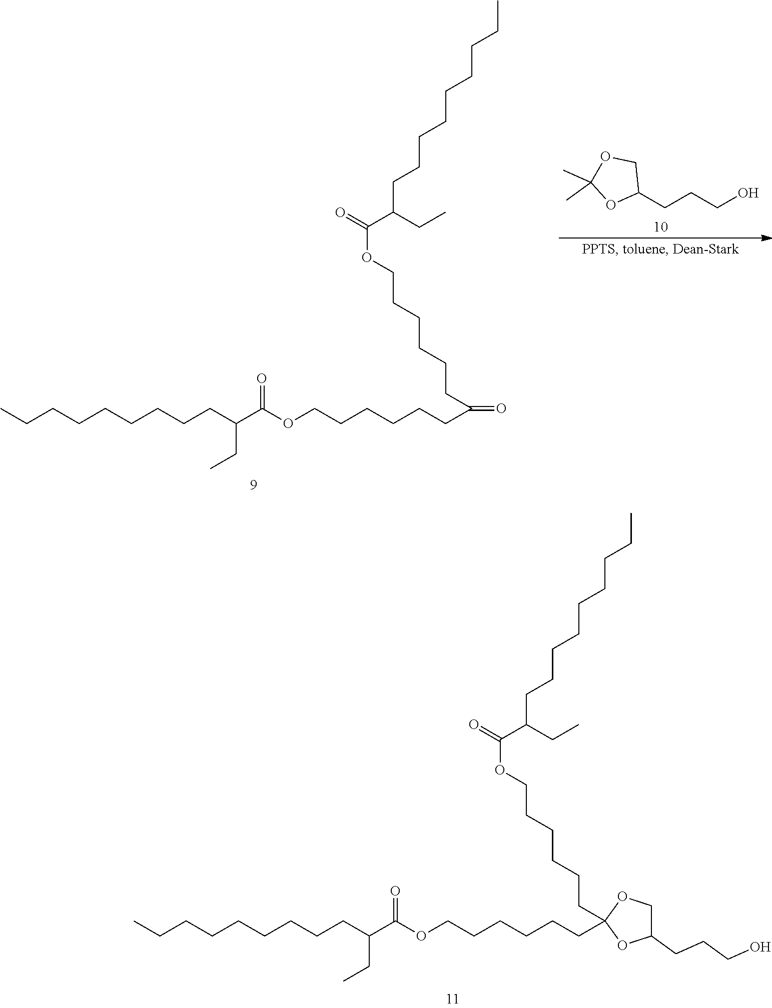 [SMILES: CCCCCCCCCC(CC)C(=O)OCCCCCCC(=O)CCCCCCOC(=O)C(CC)CCCCCCCCC ; CC1(C)OCC(CCCO)O1 ; CCCCCCCCCC(CC)C(=O)OCCCCCCC1(CCCCCCOC(=O)C(CC)CCCCCCCCC)OCC(CCCO)O1]

To a mixture of compound 9 (1.38 g, 2.22 mmol, 1 eq.) and compound 10 (887.20 mg, 5.54 mmol, 2.5 eq.) in toluene (30 mL) was added PPTS (167.00 mg, 664.53 μmol, 0.3 eq.) at 25° C., and then the mixture was stirred at 135° C. for 12 hrs equipped with a Dean-Stark trap. The reaction mixture was poured into a suspension of NaHCO₃ and water (w/w=1/1, 80 mL) and stirred for 2 min. The aqueous phase was extracted with ethyl acetate (40 mL*3). The combined organic phases were washed with brine (100 mL), dried over anhydrous Na₂SO₄, filtered and concentrated under vacuum. The residue was purified by column chromatography (SiO₂, Petroleum ether/Ethyl acetate=10/1 to 3/1) to give compound 11 (1.4 g, 67.99% yield) as a colorless oil.

¹H NMR (400 MHz, CHLOROFORM-d) δ ppm 4.07 (quin, J=5.8 Hz, 6H), 3.74-3.61 (m, 2H), 3.52-3.42 (m, 1H), 2.32-2.20 (m, 2H), 1.73-1.53 (m, 20H), 1.39-1.22 (m, 40H), 0.89 (dt, J=2.4, 7.1 Hz, 12H).

### Procedure for Preparation of Compound 12

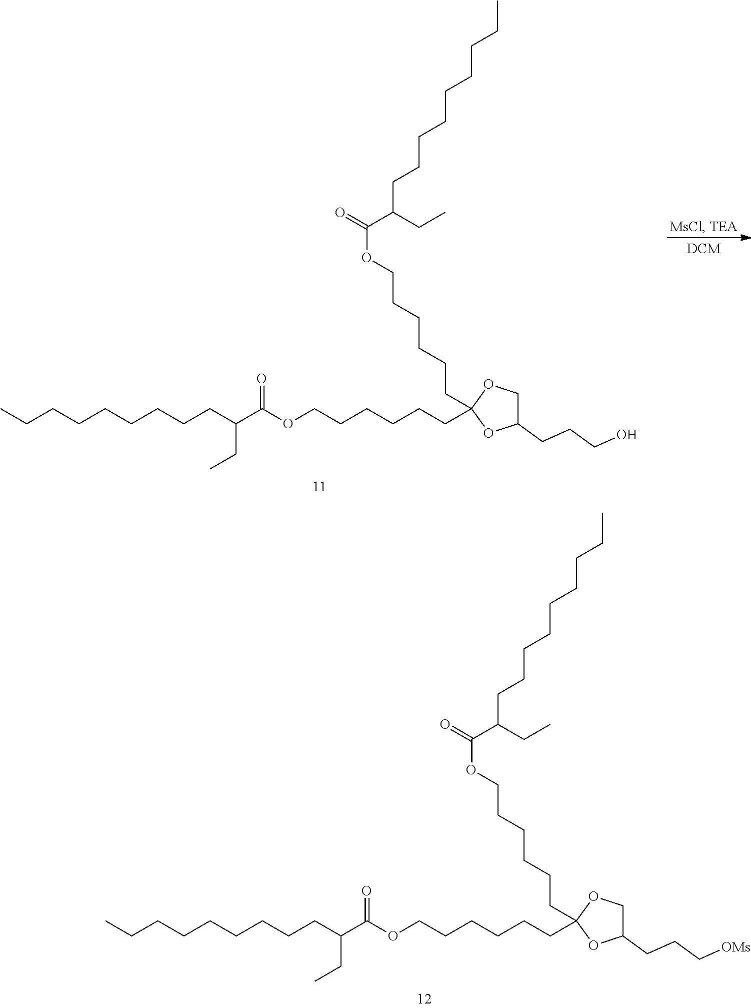 [SMILES: CCCCCCCCCC(CC)C(=O)OCCCCCCC1(CCCCCCOC(=O)C(CC)CCCCCCCCC)OCC(CCCO)O1 ; CCCCCCCCCC(CC)C(=O)OCCCCCCC1(CCCCCCOC(=O)C(CC)CCCCCCCCC)OCC(CCCOS(C)(=O)=O)O1]

To a solution of compound 11 (1.4 g, 1.93 mmol, 1 eq.) in DCM (14 mL) was added MsCl (663.48 mg, 5.79 mmol, 448.30 μL, 3 eq.) and TEA (976.83 mg, 9.65 mmol, 1.34 mL, 5 eq.) at 0° C., and then the mixture was stirred at 25° C. for 2 hrs. The reaction mixture was pourted into ice-water (60 mL) and extracted with DCM (40 mL*3). The combined organic layers were washed with brine (60 mL), dried over anhydrous Na₂SO₄, filtered and concentrated under reduced pressure. The residue was purified by column chromatography (SiO₂, PE/EA=10/1 to 5/1, eluting with 0.1% TEA) to give compound 12 (1.27 g, 81.89% yield) as a colourless oil.

¹H NMR (400 MHz, CHLOROFORM-d) δ ppm 4.36-4.21 (m, 2H), 4.11-4.02 (m, 6H), 3.51-3.43 (m, 1H), 3.02 (d, J=1.0 Hz, 3H), 2.35-2.18 (m, 2H), 1.97-1.77 (m, 2H), 1.70-1.55 (m, 18H), 1.35-1.22 (m, 40H), 0.93-0.83 (m, 12H).

### Procedure for Preparation of Compound 49

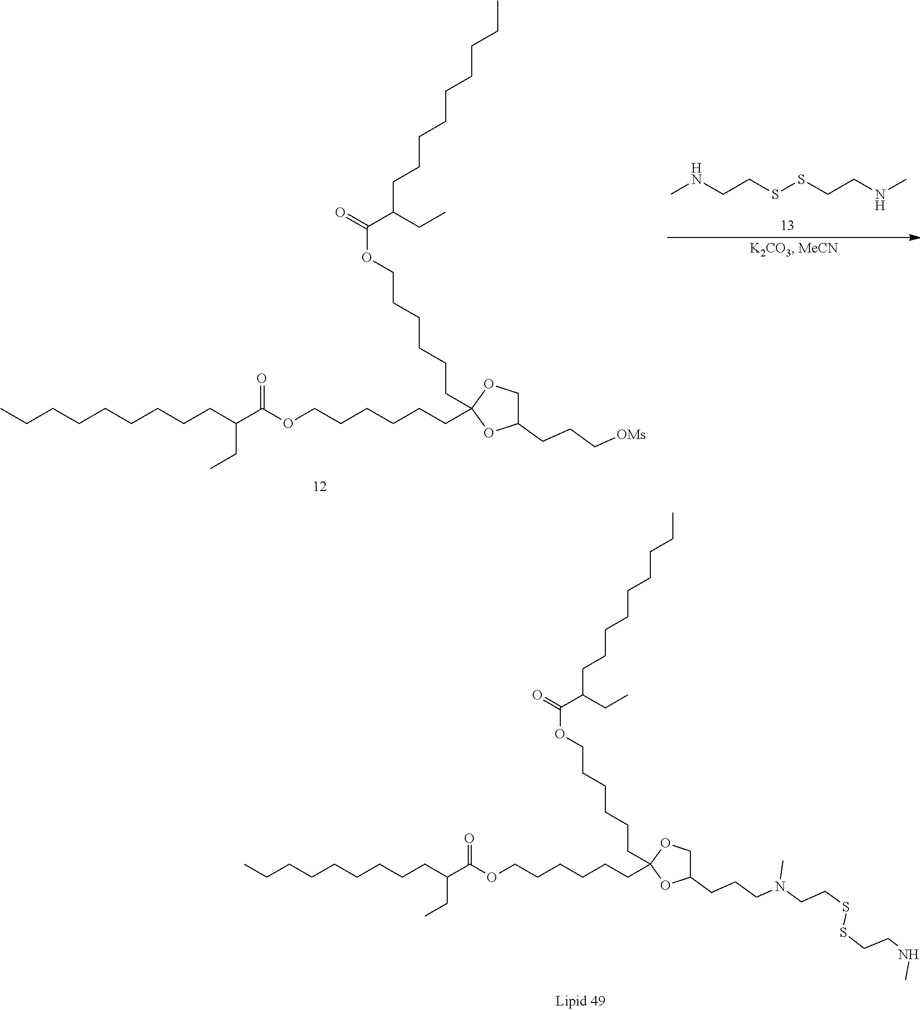

To a mixture of compound 12 (600 mg, 746.99 μmol, 1 eq.) and compound 13 (567.54 mg, 2.24 mmol, 3 eq., 2 HCl salt) in MeCN (6 mL) was added NaHCO₃ (564.77 mg, 6.72 mmol, 9 eq.) at 25° C., and then the mixture was stirred at 85° C. for 12 hrs. The reaction mixture was concentrated under reduced pressure and the residue was purified by prep-HPLC (column: X-Select CSH Phenyl-Hexyl 100*30 5u; mobile phase: [H₂O) (0.1% TFA)-EtOH]; gradient: 55%-85% B over 15.0 min), followed by prep-HPLC (column: X-Select CSH Phenyl-Hexyl 100*30 5u; mobile phase: [H₂O) (0.1% TFA)-EtOH]; gradient: 58%-88% B over 15.0 min) to give Lipid 49 (70 mg, 10.14% yield) as a yellow oil

HRMS: m/z 887.6968 [M+1]⁺; ¹H NMR (400 MHz, CHLOROFORM-d) δ ppm 11.82 11.32 (m, 1H), 10.30-9.53 (m, 2H), 4.20-3.92 (m, 6H), 3.69-2.99 (m, 13H), 2.98-2.82 (m, 6H), 2.76 (br s, 3H), 2.27 (tt, J=5.4, 8.7 Hz, 2H), 2.02-1.80 (m, 2H), 1.76-1.13 (m, 59H), 1.00-0.76 (m, 12H).

### Synthesis of Lipid 50 and Lipid 51

### Procedure for the Preparation of Compound 2

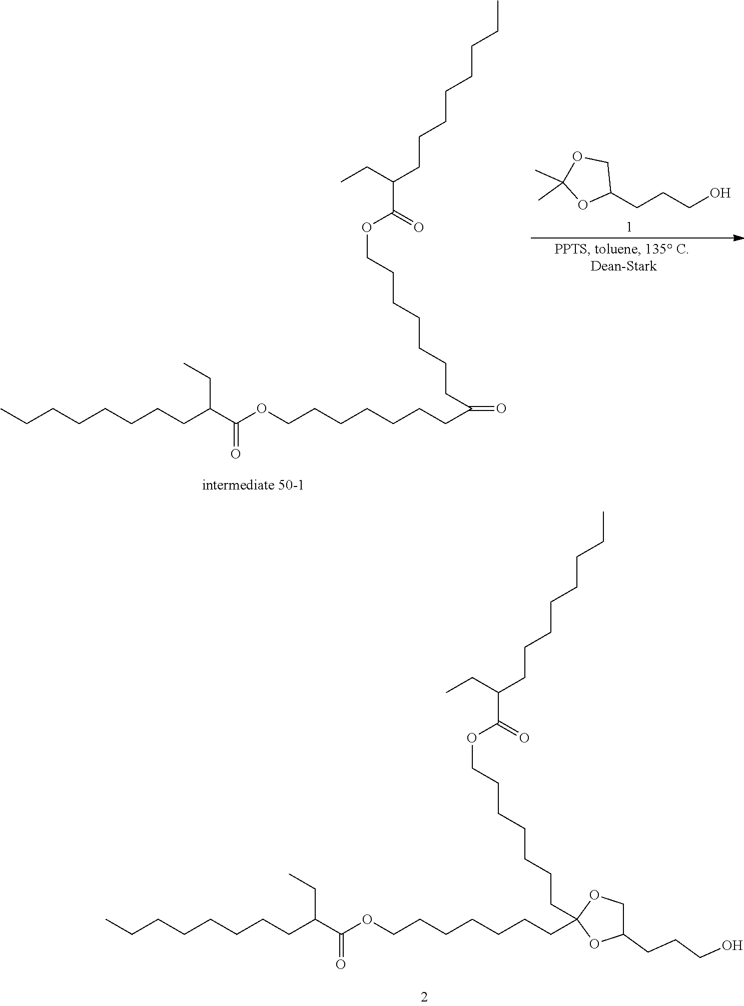 [SMILES: CCCCCCCCC(CC)C(=O)OCCCCCCCC(=O)CCCCCCCOC(=O)C(CC)CCCCCCCC ; CC1(C)OCC(CCCO)O1 ; CCCCCCCCC(CC)C(=O)OCCCCCCCC1(CCCCCCCOC(=O)C(CC)CCCCCCCC)OCC(CCCO)O1]

To a mixture of intermediate 50-1 (1.1 g, 1.77 mmol, 1 eq.) and compound 1 (707.19 mg, 4.41 mmol, 2.5 eq.) in toluene (30 mL) was added PPTS (133.11 mg, 529.70 μmol, 0.3 eq.) at 25° C., and then the mixture was stirred at 135° C. for 12 hrs equipped with a Dean-Stark trap. The reaction mixture was poured into a suspension of NaHCO₃ and water (w/w=1/1, 50 mL) and stirred for 2 min. The aqueous phase was extracted with EtOAc (100 mL*3). The combined organic phases were washed with brine (100 mL), dried over anhydrous Na₂SO₄, filtered and concentrated under vacuum. The residue was purified by column chromatography (SiO₂,

Petroleum ether/Ethyl acetate=10/1 to 3/1) to give compound 2 (1.15 g, 84.43% yield) as a colorless oil.

¹H NMR (400 MHz, CHLOROFORM-d) δ ppm 4.15-4.03 (m, 6H), 3.77-3.65 (m, 2H), 3.53-3.45 (m, 1H), 2.27 (ddd, J=3.6, 5.4, 8.8 Hz, 2H), 1.73-1.55 (m, 21H), 1.47-1.24 (m, 40H), 0.90 (dt, J=3.2, 7.1 Hz, 12H).

### Procedure for Preparation of Compound 3

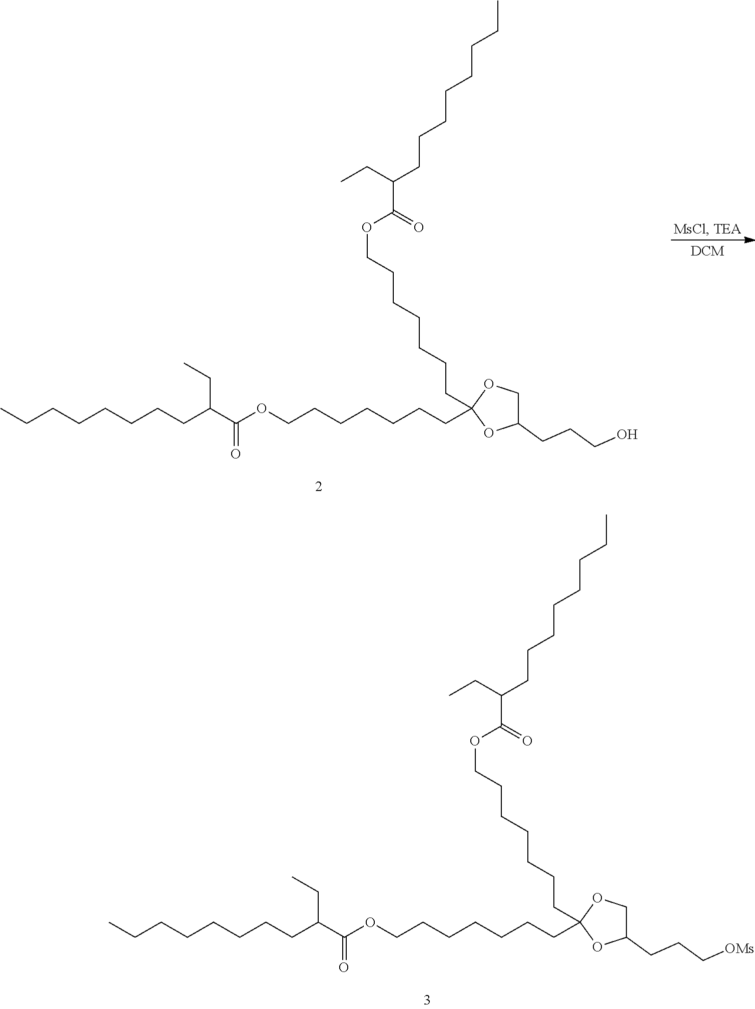 [SMILES: CCCCCCCCC(CC)C(=O)OCCCCCCCC1(CCCCCCCOC(=O)C(CC)CCCCCCCC)OCC(CCCO)O1 ; CCCCCCCCC(CC)C(=O)OCCCCCCCC1(CCCCCCCOC(=O)C(CC)CCCCCCCC)OCC(CCCOS(C)(=O)=O)O1]

To a solution of compound 2 (1.15 g, 1.59 mmol, 1 eq.) in DCM (15 mL) was added MsCl (763.01 mg, 6.66 mmol, 515.55 μL, 4.2 eq.) and TEA (802.39 mg, 7.93 mmol, 1.10 mL, 5 eq.) at 25° C., and then the mixture was stirred at 25° C. for 2 hrs. The reaction mixture was concentrated under reduced pressure. The residue was purified by column chromatography (SiO₂, Petroleum ether/Ethyl acetate=10/1 to 3/1) to give compound 3 (1.2 g, 94.20% yield) as a colorless oil.

¹H NMR (400 MHz, CHLOROFORM-d) δ ppm 4.39-4.25 (m, 2H), 4.16-4.03 (m, 6H), 3.56-3.45 (m, 1H), 3.05 (s, 3H), 2.36-2.20 (m, 2H), 2.02-1.79 (m, 2H), 1.75-1.60 (m, 12H), 1.58-1.46 (m, 6H), 1.45-1.19 (m, 40H), 0.91 (dt, J=3.2, 7.1 Hz, 12H).

### Procedure for Preparation of Lipid 50 and Lipid 51

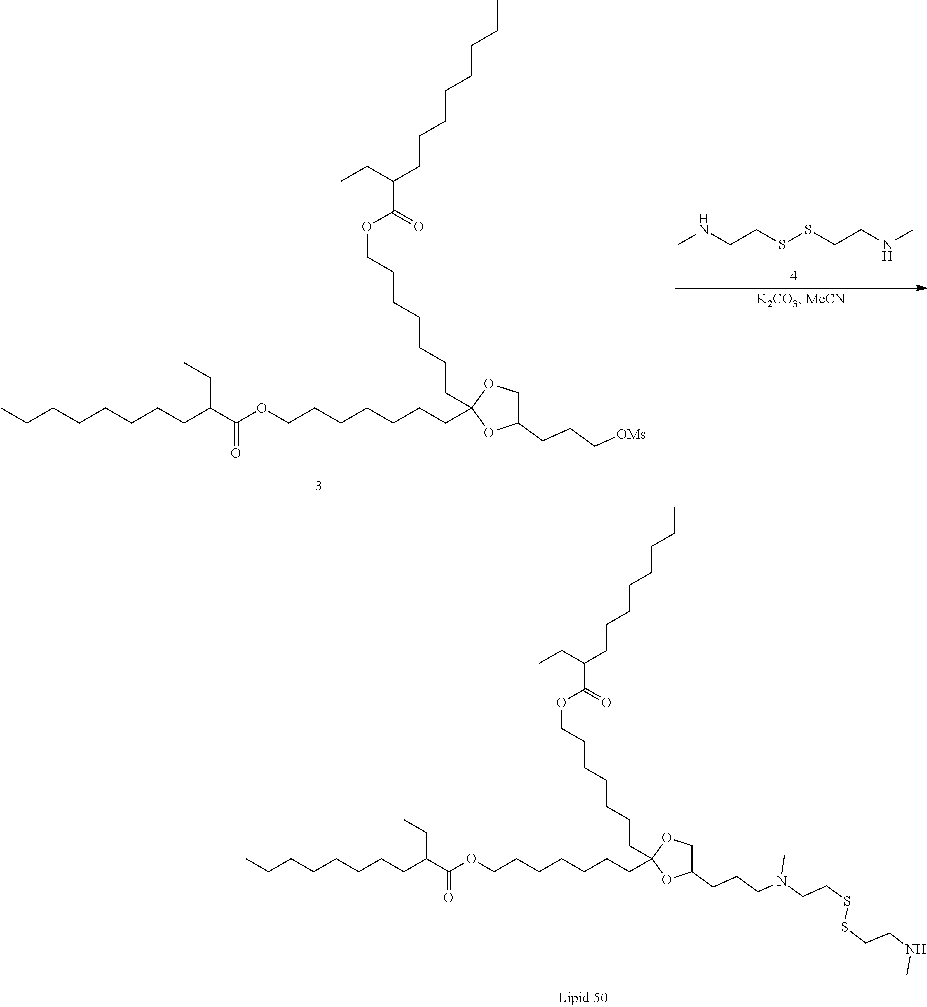

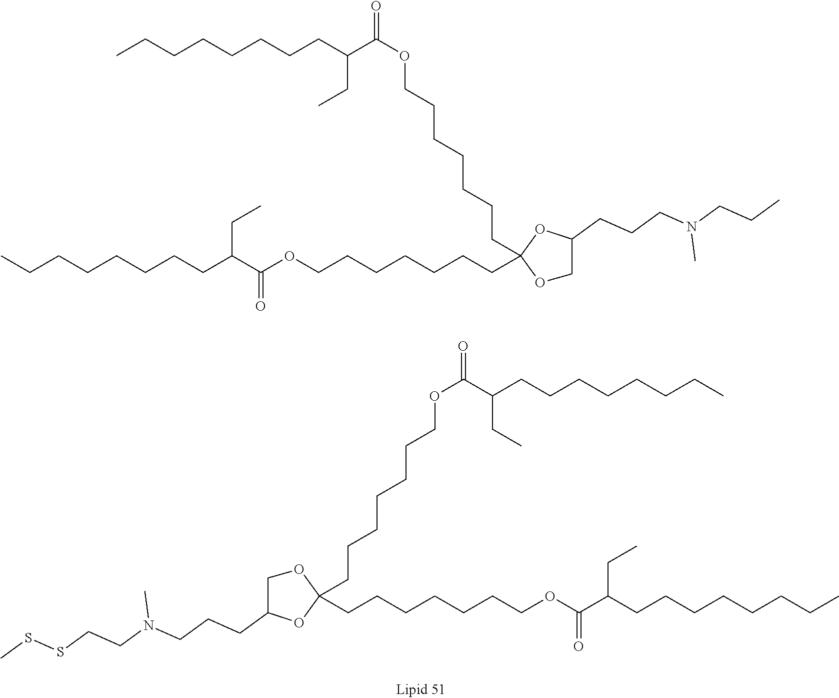

To a mixture of compound 3 (500 mg, 622.49 μmol, 1 eq.) and compound 4 (394.13 mg, 1.56 mmol, 2.5 eq., 2 HCl salt) in MeCN (5 mL) was added K₂CO₃ (258.10 mg, 1.87 mmol, 3 eq.) at 25° C., and then the mixture was stirred at 85° C. for 12 hrs. The reaction mixture was concentrated under reduced pressure and the residue was purified by prep-HPLC (column: Waters Xbridge C18 150*25 mm*5 μm; mobile phase: [H₂O (0.1% TFA)-EtOH]; gradient: 55%-85% B over 14.0 min) to give crude Lipid 50 (300 mg) as a colorless oil and crude Lipid 51 (120 mg) as a colorless oil.

The crude Lipid 50 (300 mg) was further purified by prep-NPLC (column: Welch Ultimate XB—SiOH 250*50 mm*10 μm; mobile phase: [Hexane-EtOH]; gradient: 1%-10% B over 15.0 min) to give Lipid 50 (168.2 mg, 30.44% yield) s a light-yellow oil.

HRMS: m/z 887.6938 [M+1]⁺; ¹H NMR (400 MHz, CHLOROFORM-d) δ ppm 4.16-4.00 (m, 6H), 3.52-3.41 (m, 1H), 3.14-2.76 (m, 8H), 2.55 (s, 3H), 2.50 (br t, J=7.2 Hz, 2H), 2.33 (s, 3H), 2.30-2.26 (m, 2H), 1.72-1.43 (m, 21H), 1.41-1.21 (m, 42H), 0.90 (dt, J=3.2, 7.2 Hz, 12H).

The crude Lipid 51 (120 mg) was further purified by prep-NPLC (column: Welch Ultimate XB—SiOH 250*50 mm*10 μm; mobile phase: [Hexane-EtOH]; gradient: 1%-10% B over 15.0 min) to give Lipid 51 (74 mg, 7.45% yield) as a yellow oil.

HRMS: m/z 1595.3093 [M+1]⁺; ¹H NMR (400 MHz, CHLOROFORM-d) δ ppm 4.17-3.95 (m, 12H), 3.53-3.42 (m, 2H), 2.89-2.64 (m, 6H), 2.52-2.35 (m, 4H), 2.32-2.21 (m, 8H), 1.75-1.47 (m, 40H), 1.46-1.18 (m, 84H), 0.90 (dt, J=3.2, 7.2 Hz, 24H).

### Synthesis of Lipid 52

### Procedure for Preparation of Compound 3

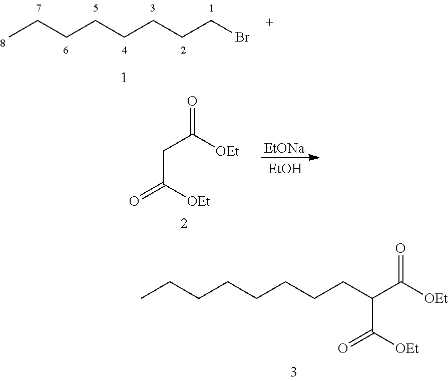

To a solution of compound 2 (10 g, 62.43 mmol, 9.48 mL, 1 eq.) and EtONa (4.25 g, 62.43 mmol, 1 eq.) in EtOH (150 mL) was added compound 1 (9.65 g, 49.95 mmol, 8.69 mL, 0.8 eq.) at 0° C., and then the mixture was stirred at 50° C. for 1 hr. The reaction mixture was poured into a mixture of EtOAc (40 mL) and water (40 mL) and then layers were separated. The aqueous phase was extracted with EtOAc (30 mL*2). The combined organic layers were washed with brine (60 mL), dried over anhydrous Na₂SO₄, filtered and concentrated under vacuum. The residue was purified by flash silica gel chromatography (0˜10%, Petroleum ether/Ethyl acetate) to give compound 3 (15.02 g, 70.66% yield) as a white solid.

LCMS: m/z 273.4 [M+1]⁺; ¹H NMR (400 MHz, CHLOROFORM-d) δ ppm 4.24-4.10 (m, 4H), 3.34-3.25 (m, 1H), 1.91-1.78 (m, 2H), 1.43-1.18 (m, 18H), 0.91-0.78 (m, 3H).

### Procedure for Preparation of Compound 4

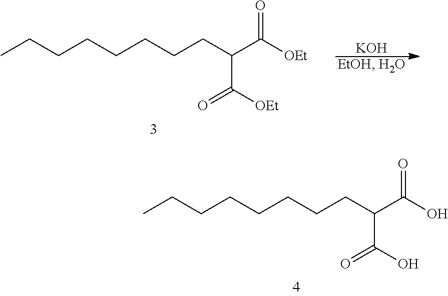

To a solution of compound 3 (10 g, 36.71 mmol, 1 eq.) in a mixed solvent of EtOH (150 mL) and H₂O (20 mL) was added KOH (8.24 g, 146.85 mmol, 4 eq.) at 25° C., and then the mixture was stirred at 80° C. for 2.5 hr. The reaction mixture was adjusted to pH=5 with 6 N HCl aqueous solution. The precipitate was collected by filtration and dried in vacuo to give compound 4 (7.62 g, 76.77% yield) as a white solid, which was used directly for the next step without further purification.

¹H NMR (400 MHz, DMSO-d₆) δ ppm 12.63 (br s, 2H), 3.17 (t, J=7.4 Hz, 1H), 1.77-1.60 (m, 2H), 1.31-1.18 (m, 12H), 0.91-0.80 (m, 3H).

### Procedure for Preparation of Compound 5

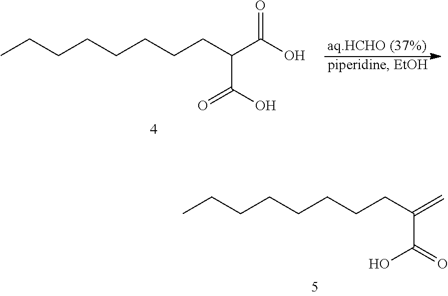

To a solution of compound 4 (7 g, 32.37 mmol, 1 eq.) and 37% HCHO aqueous solution (7.23 mL, 97.10 mmol, 3 eq.) in EtOH (80 mL) was added piperidine (12.79 mL, 129.47 mmol, 4 eq.), and then the mixture was stirred at 80° C. for 12 hrs. The reaction mixture was adjusted to pH-6 with 1 N HCl aqueous solution, and then poured into a mixture of EtOAc (400 mL) and water (400 mL). Layers were separated, and the aqueous phase was extracted with EtOAc (300 mL*2). The combined organic layers were washed with brine (600 mL), dried over anhydrous Na₂SO₄, filtered and concentrated under vacuum. The residue was purified by flash silica gel chromatography (0˜20%, Petroleum ether/Ethyl acetate) to give compound 5 (3.62 g, 48.56% yield) as a white solid.

LCMS: m/z 185.3 [M+1]⁺; ¹H NMR (400 MHz, CHLOROFORM-d) δ ppm 6.29 (s, 1H), 5.65 (s, 1H), 2.66-2.27 (m, 2H), 1.52-1.26 (m, 12H), 0.96-0.86 (m, 3H).

### Procedure for Preparation of Compound 6

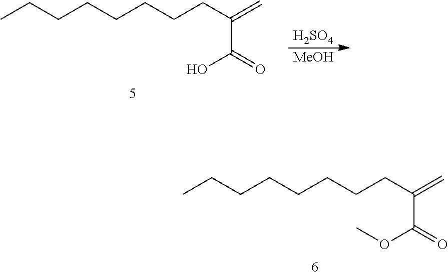

To a solution of compound 5 (2 g, 10.85 mmol, 1 eq.) in MeOH (30 mL) was added conc. H₂SO₄ (1.16 mL, 21.71 mmol, 2 eq.), and then the mixture was stirred at 60° C. for 2 hrs. The reaction mixture was concentrated under vacuum and then poured into a mixture of EtOAc (40 mL) and water (40 mL). Layers were separated and the aqueous phase was extracted with EtOAc (30 mL*2). The combined organic layers were washed with brine (60 mL), dried over anhydrous Na₂SO₄, filtered and concentrated under vacuum. The residue was purified by flash silica gel chromatography (0˜10%, Petroleum ether/Ethyl acetate) to give compound 6 (1.82 g, 67.65% yield) as a white solid.

¹H NMR (400 MHz, CHLOROFORM-d) δ ppm 6.13 (s, 1H), 5.52 (d, J=1.3 Hz, 1H), 3.75 (s, 3H), 2.29 (t, J=7.6 Hz, 2H), 1.50-1.42 (m, 2H), 1.35-1.26 (m, 10H), 0.88 (d, J=3.5 Hz, 3H).

### Procedure for Preparation of Compound 8

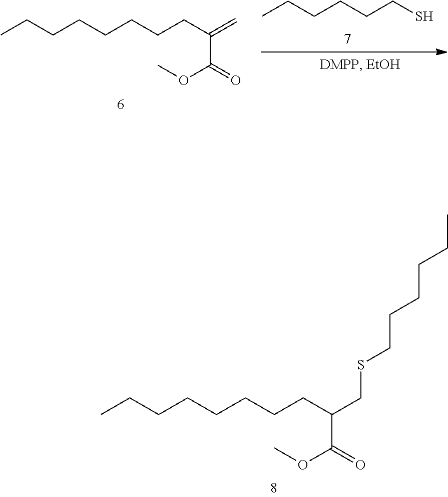

To a mixture of compound 6 (800 mg, 4.03 mmol, 1 eq.) and compound 7 (572.41 mg, 4.84 mmol, 1.2 eq.) in EtOH (15 mL) was added DMPP (111.46 mg, 806.85 μmol, 0.2 eq.), and then the mixture was stirred at 80° C. for 12 hrs. The reaction mixture was poured into a mixture of EtOAc (40 mL) and water (40 mL). Layers were separated and the aqueous phase was extracted with EtOAc (30 mL*2). The combined organic layers were washed with brine (60 mL), dried over anhydrous Na₂SO₄, filtered and concentrated under vacuum. The residue was purified by flash silica gel chromatography (0˜10%, Petroleum ether/Ethyl acetate) to give compound 8 (1.22 g, 80.25% yield) as a white solid.

LCMS: m/z 317.2 [M+1]⁺; ¹H NMR (400 MHz, CHLOROFORM-d) ¿ ppm 3.70 (s, 3H), 2.79-2.71 (m, 1H), 2.65-2.55 (m, 2H), 2.53-2.45 (m, 2H), 1.66-1.52 (m, 4H), 1.40-1.34 (m, 2H), 1.30-1.23 (m, 16H), 0.88 (dt, J=3.9, 6.8 Hz, 6H).

### Procedure for Preparation of Acid A

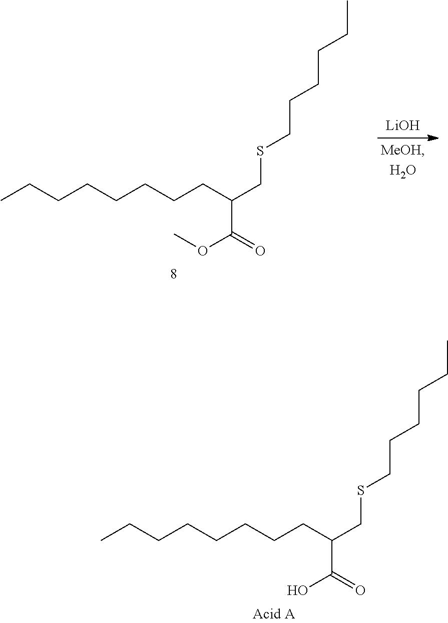

To a solution of compound 8 (4.2 g, 13.27 mmol, 1 eq.) in a mixed solvent of H₂O (10 mL) and MeOH (60 mL) was added LiOH·H₂O (2.23 g, 53.07 mmol, 4 eq.), and then the mixture was stirred at 50° C. for 2 hrs. The reaction mixture was adjusted to pH=5 with 1 N HCl aqueous solution. The precipitate was collected by filtration and dried in vacuo to give Acid A (4.82 g, 96.07% yield) as a white oil, which was used directly for the next step without further purification.

MS: m/z 301.2 [M−1]⁺; ¹H NMR (400 MHz, CHLOROFORM-d) 0 ppm 2.85-2.76 (m, 1H), 2.69-2.58 (m, 2H), 2.56-2.48 (m, 2H), 1.74-1.61 (m, 2H), 1.60-1.52 (m, 2H), 1.43-1.24 (m, 18H), 0.96-0.83 (m, 6H).

### Procedure for Preparation of Compound 10

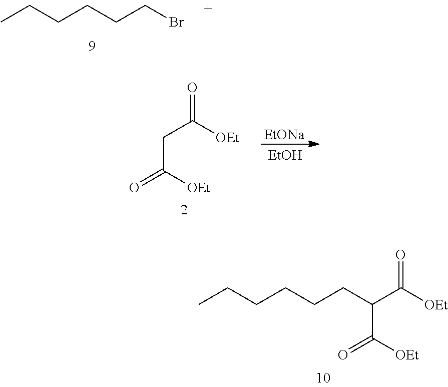

To a solution of compound 2 (11.6 g, 72.7 mmol, 1.2 eq.) in EtOH (100 mL) was added EtONa (4.12 g, 60.6 mmol, 1 eq.) at 25° C., and then added compound 9 (10.0 g, 60.6 mmol, 1 eq.) to the mixture. The mixture was stirred at 50° C. for 12 hrs. The reaction mixture was quenched by addition of water (80 mL) at 0° C., and then extracted with EtOAc (80.0 mL*3). The combined organic layers were washed with brine (30 mL*3), dried over anhydrous Na₂SO₄, filtered and concentrated under reduced pressure. The residue was purified by column chromatography (SiO₂, Petroleum ether/Ethyl acetate=I/O to 50/1) to give compound 10 (7.93 g, 48% yield) as a colorless liquid.

LCMS: m/z 267.1 [M+23]; 1H NMR (400 MHz, CHLOROFORM-d) δ ppm 4.18 (qd, J=7.2, 1.2 Hz, 4H), 3.30 (t, J=7.6 Hz, 1H), 1.83-1.92 (m, 2H), 1.22-1.32 (m, 14H), 0.83-0.90 (m, 3H).

### Procedure for Preparation of Compound 11

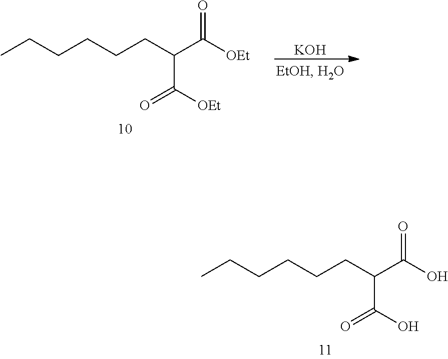 [SMILES: CCCCCCC(C(=O)OCC)C(=O)OCC ; "KOH" ; CCCCCCC(C(=O)O)C(=O)O]

To a solution of compound 10 (7.92 g, 32.4 mmol, 1 eq.) in a mixed solvent of EtOH (120 mL) and H₂O (40 mL) was added KOH (7.27 g, 130 mmol, 4 eq.) at 25° C., and then the mixture was stirred at 80° C. for 2 hrs. The reaction mixture was quenched by addition of 1N HCl aqueous solution (100 mL) at 0° C., and then diluted with water (50 mL) and extracted with EtOAc (150 mL*3). The combined organic layers were washed with brine (50 mL*2), dried over anhydrous Na₂SO₄, filtered and concentrated under reduced pressure to give crude compound 11 (5.988 g) as a white solid, which was used directly for the next step without further purification.

¹H NMR (400 MHz, DMSO-de) 5 ppm 11.97-13.31 (m, 2H), 3.18 (t, J=7.4 Hz, 1H), 1.63-1.75 (m, 2H), 1.25 (s, 8H), 0.82-0.88 (m, 3H).

### Procedure for Preparation of Compound 12

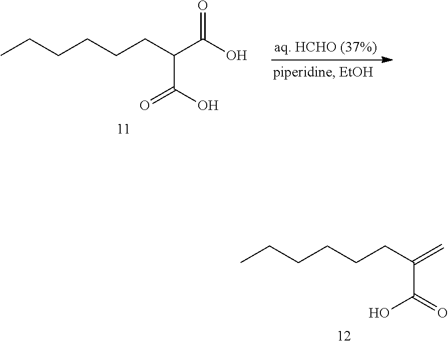

To a solution of compound 11 (5.99 g, 31.8 mmol, 1 eq.) in EtOH (60 mL) was added piperidine (10.8 g, 127 mmol, 12.6 mL, 4 eq.) and 37% HCHO aqueous solution (7.11 mL, 95.4 mmol, 3 eq.) at 25° C., and then the mixture was stirred at 80° C. for 12 hrs. The reaction mixture was quenched by addition of 1N HCl (50 mL) at 0° C., and then diluted with water (50 mL) and extracted with DCM (50 mL*3). The combined organic layers were washed with brine (20 mL*2), dried over anhydrous Na₂SO₄, filtered and concentrated under reduced pressure. The residue was purified by column chromatography (SiO₂, Petroleum ether/Ethyl acetate=I/O to 50/1) to give compound 12 (3.04 g, 55% yield) as a yellow liquid.

¹H NMR (400 MHz, CHLOROFORM-d) δ ppm 10.24-13.46 (m, 1H), 6.30 (s, 1H), 5.65 (d, J=1.2 Hz, 1H), 2.31 (t, J=7.6 Hz, 2H), 1.44-1.54 (m, 2H), 1.26-1.38 (m, 6H), 0.86-0.93 (m, 3H).

### Procedure for Preparation of Compound 13

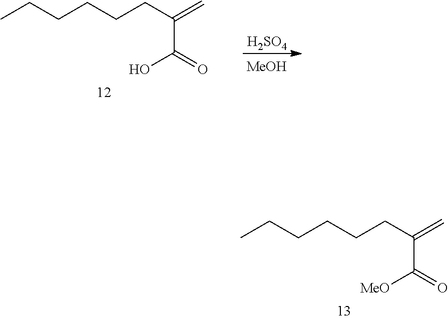

To a solution of compound 12 (2.28 g, 14.6 mmol, 1 eq.) in MeOH (34 mL) was added conc. H₂SO₄ (1.56 mL, 29.2 mmol, 2 eq.), and then the mixture was stirred at 60° C. for 2 hrs. The reaction mixture was concentrated under reduced pressure. The residue was diluted with water (10 mL) and extracted with EtOAc (20 mL*3). The combined organic layers were washed with brine (20 mL), dried over anhydrous Na₂SO₄, filtered and concentrated under reduced pressure to give crude compound 13 (2.02 g) as a yellow oil, which was used directly for the next step without further purification.

LCMS: m/z 171.1 [M+1]⁺; ¹H NMR (400 MHz, CHLOROFORM-d) δ ppm 6.13 (s, 1H), 5.53 (d, J=1.6 Hz, 1H), 3.76 (s, 3H), 2.30 (t, J=7.6 Hz, 2H), 1.42-1.50 (m, 2H), 1.29-1.36 (m, 6H), 0.86-0.91 (m, 3H).

### Procedure for Preparation of Compound 14

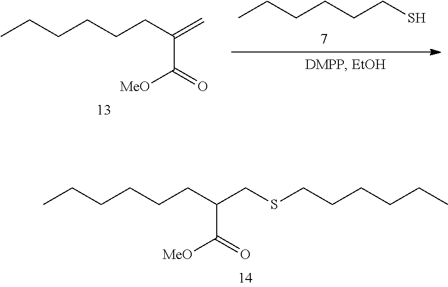

To a solution of compound 13 (2.02 g, 11.8 mmol, 1 eq.) in EtOH (40 mL) was added DMPP (328 mg, 2.37 mmol, 0.2 eq.) and compound 7 (2.10 g, 17.8 mmol, 1.5 eq.) at 25° C., and then the mixture was stirred at 80° C. for 3 hrs under N₂ atmosphere. The reaction mixture was concentrated under reduced pressure and the residue was purified by column chromatography (SiO₂, Commercial hexanes: Ethyl acetate=I/O to 50/1) to give compound 14 (2.10 g, 55% yield) as a colorless oil.

LCMS: m/z 289.2 [M+1]; 1H NMR (400 MHz, CHLOROFORM-d) δ ppm 3.71 (s, 3H), 2.72-2.81 (m, 1H), 2.56-2.66 (m, 2H), 2.51 (t, J=7.4 Hz, 2H), 1.53-1.63 (m, 4H), 1.26-1.41 (m, 14H), 0.86-0.91 (m, 7H).

### Procedure for Preparation of Acid B

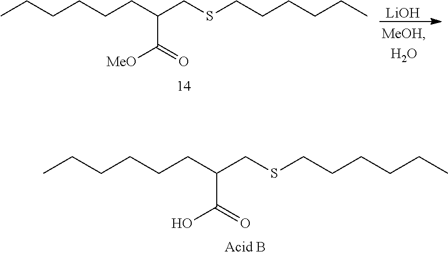

To a solution of compound 14 (2.10 g, 7.27 mmol, 1 eq.) in a mixed solvent of MeOH (17.5 mL) and H₂O (3.5 mL) was added LiOH·H₂O (916 mg, 21.8 mmol, 3 eq.) at 25° C., and then the mixture was stirred at 50° C. for 12 hrs. The reaction mixture was quenched by addition of 1N HCl (20 mL) at 0° C., and then diluted with water (10 mL) and extracted with EtOAc (20 mL*3). The combined organic layers were washed with brine (20 mL*2), dried over anhydrous Na₂SO₄, filtered and concentrated under reduced pressure. The residue was purified by column chromatography (SiO₂, PE: Ethyl acetate=1/0 to 1/10) to give Acid B (1.10 g, 50% yield) as a yellow oil.

LCMS: m/z 275.2 [M+1]; 1H NMR (400 MHz, CHLOROFORM-d) δ ppm 2.74-2.84 (m, 1H), 2.58-2.69 (m, 2H), 2.53 (t, J=7.4 Hz, 2H), 1.53-1.73 (m, 4H), 1.25-1.43 (m, 14H), 0.85-0.93 (m, 6H).

### Procedure for Preparation of Compound 15

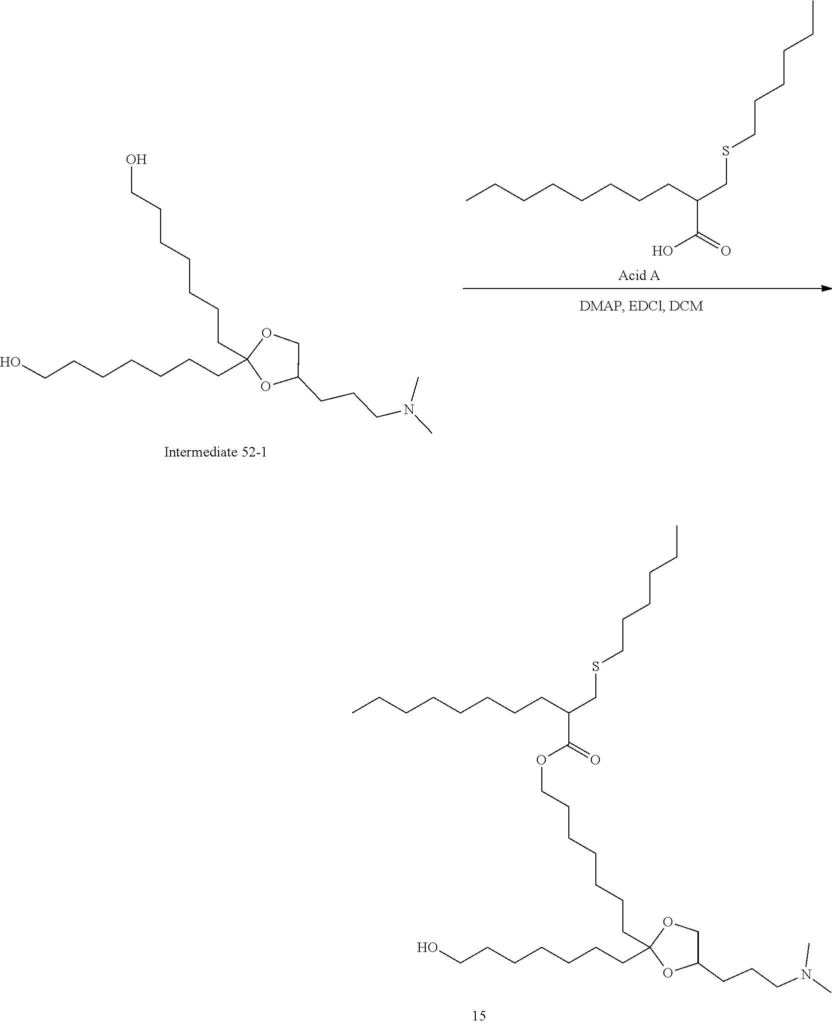 [SMILES: CN(C)CCCC1COC(CCCCCCCO)(CCCCCCCO)O1 ; CCCCCCCCC(CSCCCCCC)C(=O)O ; CCCCCCCCC(CSCCCCCC)C(=O)OCCCCCCCC1(CCCCCCCO)OCC(CCCN(C)C)O1]

To a mixture of Acid A (390.25 mg, 1.29 mmol, 1 eq.) and intermediate 52-1 (500 mg, 1.29 mmol, 1 eq.) in DCM (6 mL) was added DMAP (189.12 mg, 1.55 mmol, 1.2 eq.) and EDCI (296.75 mg, 1.55 mmol, 1.2 eq.), and then the mixture was stirred at 25° C. for 3 hrs. The reaction mixture was poured into a mixture of EtOAc (40 mL) and water (40 mL). Layers were separated and the aqueous phase was extracted with EtOAc (30 mL*2). The combined organic layers were washed with brine (60 mL), dried over anhydrous Na₂SO₄, filtered and concentrated under vacuum. The residue was purified by flash silica gel chromatography (0˜10%, Petroleum ether/Ethyl acetate) to give compound 15 (682 mg, 71.58% yield) as a white solid.

LCMS: m/z 672.7 [M+1]⁺; ¹H NMR (400 MHz, CHLOROFORM-d) δ ppm 4.21-3.98 (m, 4H), 3.72-3.58 (m, 1H), 3.51-3.41 (m, 1H), 3.17-2.98 (m, 7H), 2.78-2.45 (m, 9H), 1.88-1.41 (m, 18H), 1.39-1.20 (m, 30H), 1.03-0.80 (m, 6H).

### Procedure for Preparation of Lipid 52

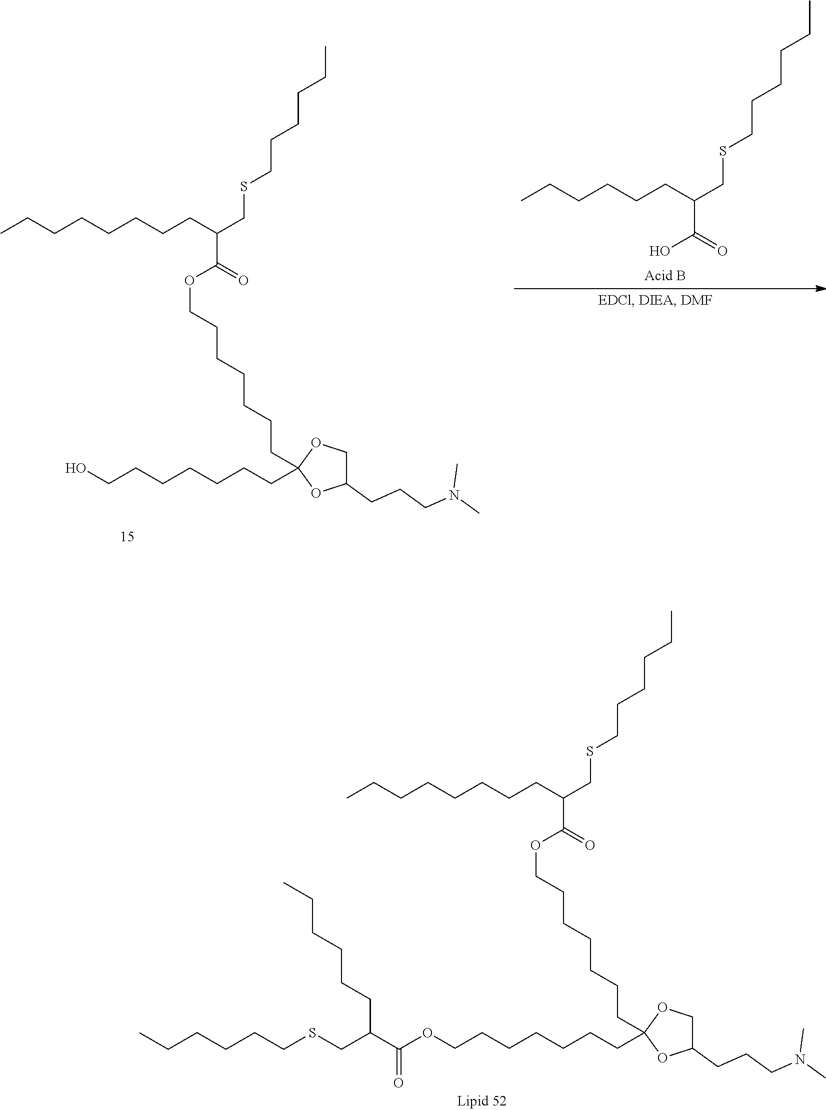 [SMILES: CCCCCCCCC(CSCCCCCC)C(=O)OCCCCCCCC1(CCCCCCCO)OCC(CCCN(C)C)O1 ; CCCCCCSCC(CCCCCC)C(=O)O ; CCCCCCCCC(CSCCCCCC)C(=O)OCCCCCCCC1(CCCCCCCOC(=O)C(CCCCCC)CSCCCCCC)OCC(CCCN(C)C)O1]

To a mixture of Acid B (816.73 mg, 2.98 mmol, 4 eq.) and compound 15 (500 mg, 743.94 μmol, 1 eq.) in DCM (5 mL) was added DIEA (288.45 mg, 2.23 mmol, 3 eq.) and EDCI (285.23 mg, 1.49 mmol, 2 eq.), and then the mixture was stirred at 25° C. for 2.5 hr. The reaction mixture was poured into a mixture of EtOAc (40 mL) and water (40 mL). Layers were separated and the aqueous phase was extracted with EtOAc (30 mL*2). The combined organic layers were washed with brine (60 mL), dried over anhydrous Na₂SO₄, filtered and concentrated under vacuum. The residue was purified by prep-HPLC (column: X-Select CSH Phenyl-Hexyl 100*30 5u; mobile phase: [H₂O (0.225% FA)-EtOH]; gradient: 60%-90% B over 16.0 min), followed by prep-HPLC (column: X-Select CSH Phenyl-Hexyl 100*30 5u; mobile phase: [H₂O (0.225% FA)-ACN]; gradient: 62%-92% B over 20.0 min) to give Lipid 52 (260 mg, 37.64% yield) as a colourless oil.

HRMS: m/z 928.7473 [M+1]⁺; ¹H NMR (400 MHz, CHLOROFORM-d) δ ppm 4.15-3.92 (m, 6H), 3.50-3.42 (m, 1H), 2.81-2.71 (m, 2H), 2.67-2.55 (m, 4H), 2.50 (d, J=7.4 Hz, 3H), 2.39-2.30 (m, 2H), 2.27 (s, 6H), 1.71-1.49 (m, 20H), 1.40-1.21 (m, 48H), 0.95-0.82 (m, 12H).

### Synthesis of Lipid 53

### Procedure for Preparation of Compound 3

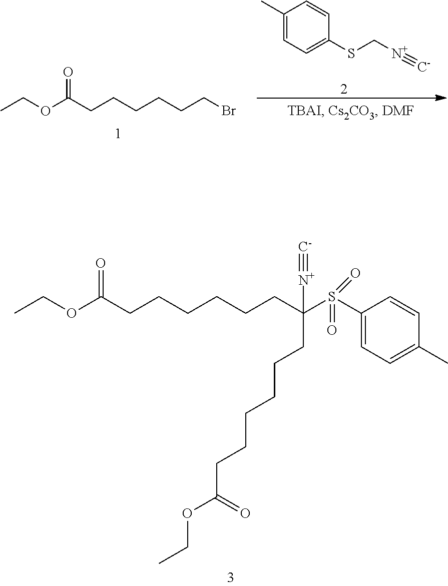

To a solution of compound 1 (1 g, 4.22 mmol, 2.1 eq.) in DMF (5 mL) was added Cs₂CO₃ (1.96 g, 6.02 mmol, 3 eq.), TBAI (74.2 mg, 201 μmol, 0.1 eq.) and compound 2 (392 mg, 2.01 mmol, 1 eq.), and then the mixture was stirred at 50° C. for 1 hr. The reaction mixture was diluted with water (20 mL) and extracted with EtOAc (25 mL*3). The combined organic layers were washed with brine (10 mL*3), dried over anhydrous Na₂SO₄, filtered and concentrated under reduced pressure to give crude compound 3 (1 g) as a yellow oil.

¹H NMR (400 MHz, CHLOROFORM-d) δ ppm 7.82-7.89 (m, 2H), 7.39-7.46 (m, 2H), 4.12 (q, J=7.2 Hz, 4H), 2.43-2.53 (m, 3H), 2.28 (t, J=7.4 Hz, 4H), 1.79-2.06 (m, 4H), 1.59-1.70 (m, 4H), 1.29-1.58 (m, 12H), 1.25 (t, J=7.2 Hz, 6H).

### Procedure for Preparation of Compound 4

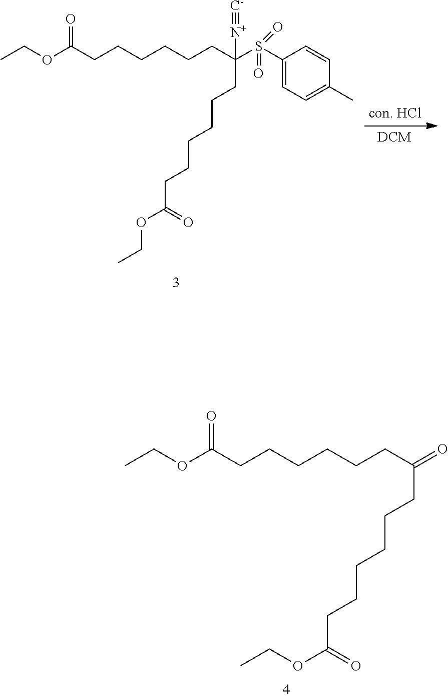

To a solution of compound 3 (1 g, 1.97 mmol, 1 eq.) in DCM (7.50 mL) was added conc. HCl (2.5 mL), and then the mixture was stirred at 25° C. for 2 hrs. The reaction mixture was diluted with water (20 mL) and extracted with DCM (25 mL*3). The combined organic layers were washed with brine (10 mL*3), dried over anhydrous Na₂SO₄, filtered and concentrated under reduced pressure. The residue was purified by column chromatography (SiO₂, Petroleum ether/Ethyl acetate=I/O to 10/1) to give compound 4 (460 mg, 61% yield) as a yellow oil.

LCMS: m/z 343.2 [M+1]⁺; ¹H NMR (400 MHz, CHLOROFORM-d) δ ppm 4.12 (q, J=7.2 Hz, 4H), 2.38 (t, J=7.4 Hz, 4H), 2.28 (t, J=7.6 Hz, 4H), 1.52-1.66 (m, 8H), 1.23-1.35 (m, 14H).

### Procedure for Preparation of Compound 6

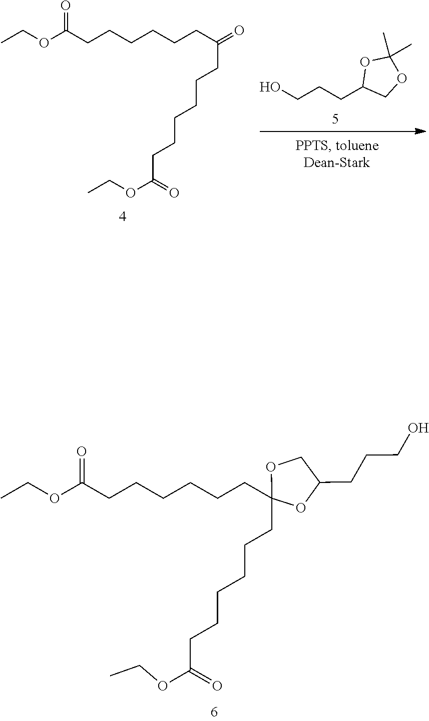 [SMILES: CCOC(=O)CCCCCCC(=O)CCCCCCC(=O)OCC ; CC1(C)OCC(CCCO)O1 ; CCOC(=O)CCCCCCC1(CCCCCCC(=O)OCC)OCC(CCCO)O1]

To a solution of compound 4 (460 mg, 1.34 mmol, 1 eq.) in toluene (5 mL) was added PPTS (169 mg, 672 μmol, 0.5 eq.) and compound 5 (538 mg, 3.36 mmol, 2.5 eq.), and then the mixture was stirred at 135° C. for 12 hrs equipped with a Dean-Stark trap. The reaction mixture was poured into sat. NaHCO₃ (aq, 60 mL) and stirred for 2 mins. The aqueous phase was extracted with DCM (30 mL*3). The combined organic phases were dried over anhydrous Na₂SO₄, filtered and concentrated under vacuum. The residue was purified by flash silica gel chromatography (ISCO®; 20 g SepaFlash® Silica Flash Column, Eluent of 0˜15% Ethyl acetate/Petroleum ethergradient @ 50 mL/min) to give compound 6 (201 mg, 30% yield) as a yellow oil.

¹H NMR (400 MHz, CHLOROFORM-d) δ ppm 4.01-4.20 (m, 6H), 3.63-3.75 (m, 2H), 3.43-3.52 (m, 1H), 2.24-2.34 (m, 4H), 1.56-1.69 (m, 12H), 1.32 (d, J=3.6 Hz, 12H), 1.26 (t, J=7.0 Hz, 6H).

### Procedure for Preparation of Compound 7

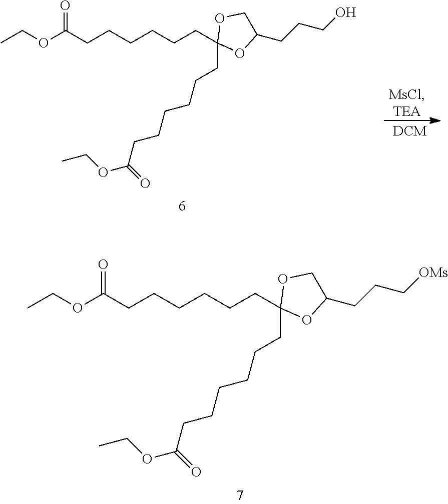

To a solution of compound 6 (200 mg, 450 μmol, 1 eq.) in DCM (3 mL) was added MsCl (155 mg, 1.35 mmol, 104 aL, 3 eq.) and TEA (228 mg, 2.25 mmol, 313 μL, 5 eq.) at 0° C., and then the mixture was stirred at 25° C. for 1 hr. The reaction mixture was concentrated under reduced pressure. The residue was purified by flash silica gel chromatography (ISCOR; 4 g SepaFlash® Silica Flash Column, Eluent of 0˜20% Ethyl acetate/Petroleum ethergradient @ 25 mL/min) to give compound 7 (178 mg, 68% yield) as s yellow oil.

¹H NMR (400 MHz, CHLOROFORM-d) δ ppm 4.22-4.33 (m, 2H), 4.12 (q, J=7.2 Hz, 4H), 4.01-4.09 (m, 2H), 3.42-3.50 (m, 1H), 3.14 (s, 2H), 3.01 (s, 3H), 2.28 (t, J=7.6 Hz, 4H), 1.78-1.96 (m, 2H), 1.52-1.70 (m, 10H), 1.31 (s, 12H), 1.25 (t, J=7.2 Hz, 6H).

### Procedure for Preparation of Compound 8

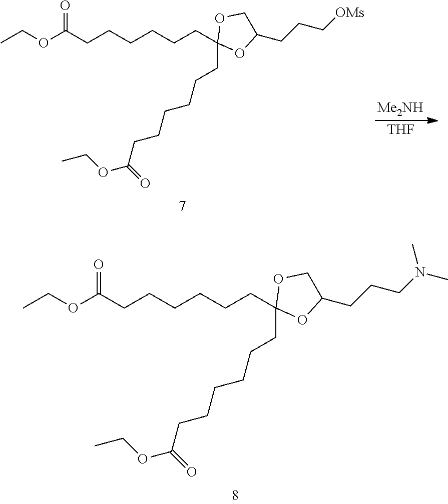 [SMILES: "Me2NH" ; CCOC(=O)CCCCCCC1(CCCCCCC(=O)OCC)OCC(CCCOS(C)(=O)=O)O1 ; CCOC(=O)CCCCCCC1(CCCCCCC(=O)OCC)OCC(CCCN(C)C)O1 ; "THF"]

To a solution of compound 7 (178 mg, 341 μmol, 1 eq.) in THF (2 mL) was added Me₂NH (2 M THE solution, 19.7 mL), and then the mixture was stirred at 55° C. for 12 hrs. The reaction mixture was concentrated under reduced pressure. The residue was purified by flash silica gel chromatography (ISCO®; 4 g SepaFlash® Silica Flash Column, Eluent of 0˜10% DCM/MeOH gradient @ 18 mL/min) to give compound 8 (109 mg, 66% yield) as a yellow oil.

LCMS: m/z 472.5 [M+1]⁺; ¹H NMR (400 MHz, CHLOROFORM-d) δ ppm 3.99-4.17 (m, 6H), 3.39-3.48 (m, 1H), 2.25-2.33 (m, 6H), 2.23 (s, 6H), 1.45-1.66 (m, 12H), 1.27-1.43 (m, 12H), 1.25 (t, J=7.2 Hz, 6H).

### Procedure for Preparation of Compound 9

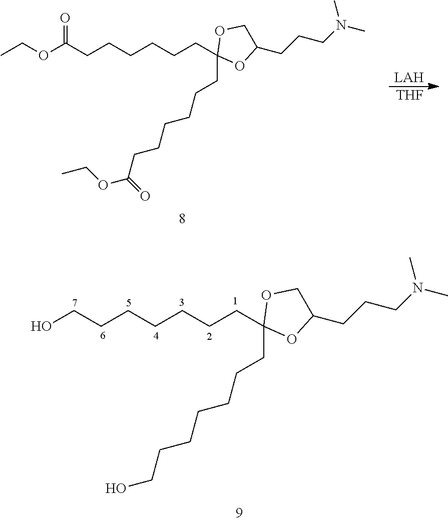 [SMILES: CCOC(=O)CCCCCCC1(CCCCCCC(=O)OCC)OCC(CCCN(C)C)O1 ; "LAH" ; CN(C)CCCC1COC(CCCCCCCO)(CCCCCCCO)O1 ; "THF"]

To a solution of compound 8 (109 mg, 231 μmol, 1 eq.) in THF (1 mL) was added LiAlH₄ (2.5 M THF solution, 462 μL, 5 eq.) dropwise slowly, and then the mixture was stirred at 20° C. for 1 hr under N₂ atmosphere. The reaction mixture was quenched by addition of sat. Na₂SO₄ aqueous solution (0.7 mL), and then dried over anhydrous Na₂SO₄, filtered and concentrated under reduced pressure to give crude compound 9 (90 mg) as a yellow oil.

LCMS: m/z 388.4 [M+1]⁺; ¹H NMR (400 MHz, CHLOROFORM-d) δ ppm 3.96-4.11 (m, 2H), 3.59 (t, J=6.6 Hz, 4H), 3.44 (t, J=7.0 Hz, 1H), 2.23-2.33 (m, 2H), 2.20 (s, 6H), 1.54 (dd, J=14.0, 6.4 Hz, 12H), 1.30 (s, 16H).

### Procedure for Preparation of Lipid 53

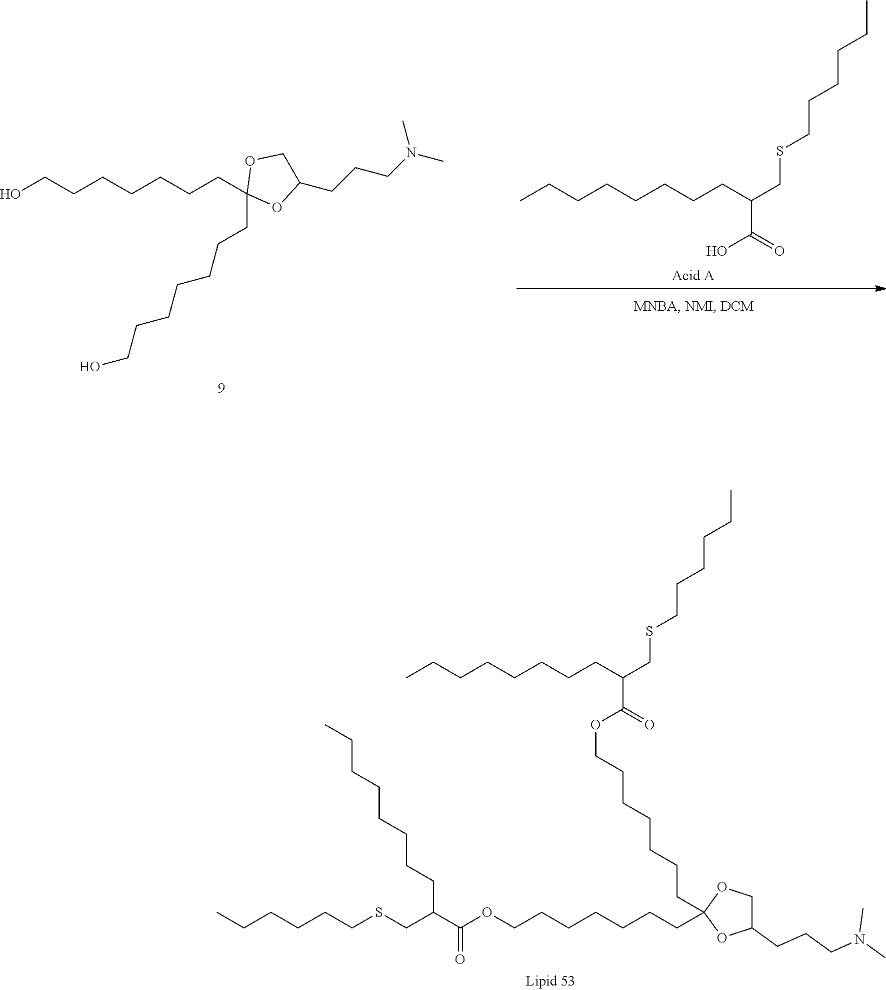 [SMILES: CN(C)CCCC1COC(CCCCCCCO)(CCCCCCCO)O1 ; CCCCCCCCC(CSCCCCCC)C(=O)O ; CCCCCCCCC(CSCCCCCC)C(=O)OCCCCCCCC1(CCCCCCCOC(=O)C(CCCCCCCC)CSCCCCCC)OCC(CCCN(C)C)O1]

To a mixture of compound 9 (90 mg, 232 μmol, 1 eq.) and Acid A (211 mg, 697 μmol, 3 eq.) in DCM (1 mL) was added NMI (95.2 mg, 1.16 mmol, 5 eq.) and MNBA (240 mg, 697 μmol, 3 eq.), and then the mixture was stirred at 25° C. for 12 hrs. The reaction mixture was concentrated under reduced pressure. The residue was purified by flash silica gel chromatography (ISCOR; 20 g SepaFlash® Silica Flash Column, Eluent of 3˜10% PE/EA gradient @ 40 mL/min), followed by prep-HPLC (column: X-Select CSH Phenyl-Hexyl 100*30 5 μm; mobile phase: [H₂O (0.225% FA)-EtOH]; gradient: 62%-94% B over 17.0 min), and then purified by prep-HPLC (column: X-Select CSH Phenyl-Hexyl 100*30 5u; mobile phase: [H₂O (0.225% FA)-EtOH]; gradient: 65%-95% B over 15.0 min) to give Lipid 53 (256.09 mg, 62% yield) as a colorless oil.

HRMS: m/z 956.7797 [M+1]⁺; ¹H NMR (400 MHz, CHLOROFORM-d) δ ppm 8.55 (s, 1H), 4.10 (t, J=6.6 Hz, 4H), 4.01-4.08 (m, 2H), 3.41-3.50 (m, 1H), 2.71-2.81 (m, 2H), 2.54˜ 2.66 (m, 4H), 2.51 (t, J=7.4 Hz, 4H), 2.33 (t, J=6.0 Hz, 2H), 2.26 (s, 6H), 1.53-1.67 (m, 18H), 1.20-1.42 (m, 54H), 0.85-0.92 (m, 12H).

### Synthesis of Lipid 54

### Procedure for Preparation of Compound 4

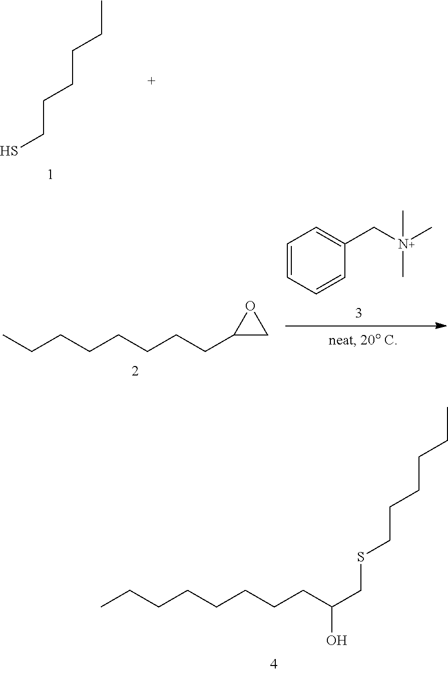

To a mixture of compound 1 (1 g, 8.46 mmol, 1 eq.) and compound 2 (1.32 g, 8.46 mmol, 1 eq.) was added compound 3 (42.43 mg, 253.72 μmol, 0.03 eq.), and then the mixture was stirred at 25° C. for 12 hrs. The reaction mixture was poured into ice-water (w/w=1/1, 30 mL) and stirred for 2 mins. The aqueous phase was extracted with EtOAc (20 mL*3). The combined organic phases were washed with brine (20 mL), dried over anhydrous Na₂SO₄, filtered and concentrated under vacuum. The residue was purified by column chromatography (SiO₂, Petroleum ether/Ethyl acetate=20/1 to 10/1) to give compound 4 (2.2 g, 85.29% yield) as a colourless oil.

¹H NMR (400 MHz, CHLOROFORM-d) δ ppm 3.76-3.61 (m, 1H), 2.78 (dd, J=3.2, 13.6 Hz, 1H), 2.56 (t, J=7.2 Hz, 2H), 2.47 (dd, J=9.2, 13.6 Hz, 1H), 1.67-1.42 (m, 8H), 1.40-1.27 (m, 14H), 0.99-0.87 (m, 6H).

### Procedure for Preparation of Compound 6

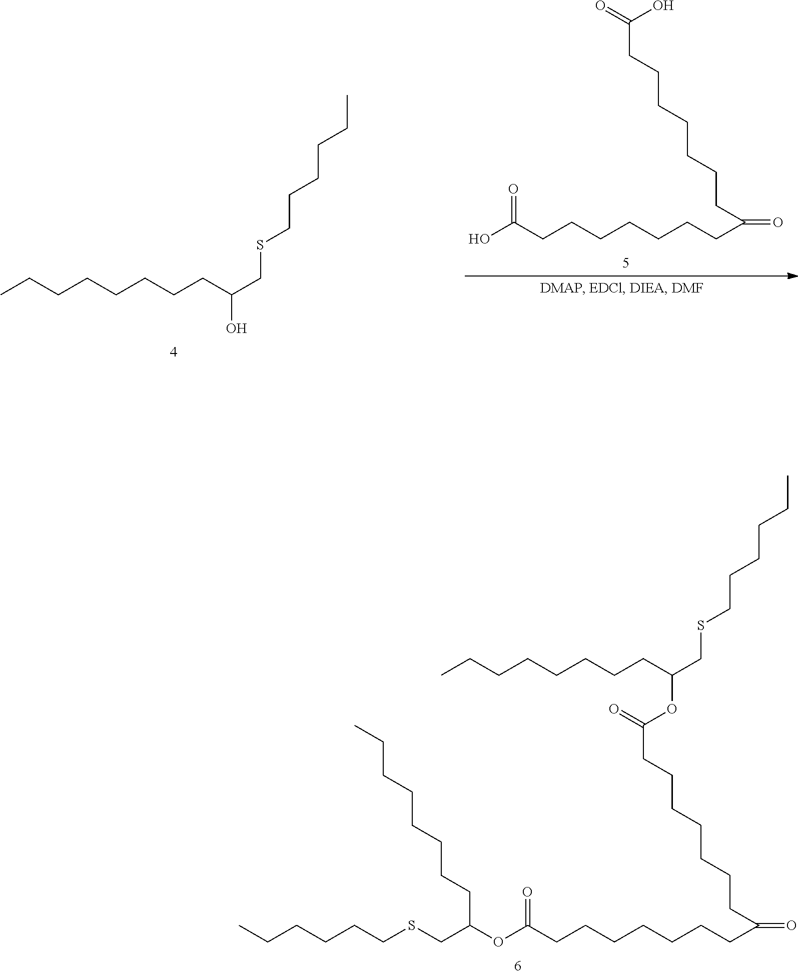 [SMILES: CCCCCCCCC(O)CSCCCCCC ; O=C(O)CCCCCCCC(=O)CCCCCCCC(=O)O ; CCCCCCCCC(CSCCCCCC)OC(=O)CCCCCCCC(=O)CCCCCCCC(=O)OC(CCCCCCCC)CSCCCCCC]

To a mixture of compound 5 (1.2 g, 3.82 mmol, 1 eq.) and compound 4 (2.10 g, 7.63 mmol, 2 eq.) in DCM (12 mL) was added DIEA (2.96 g, 22.90 mmol, 3.99 mL, 6 eq.), DMAP (1.17 g, 9.54 mmol, 2.5 eq.) and EDCI (2.19 g, 11.45 mmol, 3 eq.) at 25° C., and then the mixture was stirred at 25° C. for 12 hrs. The reaction mixture was concentrated under reduced pressure. The residue was purified by column chromatography (SiO₂, Petroleum ether/Ethyl acetate=20/1 to 10/1) to give compound 6 (2.8 g, 87.78% yield) as a colorless oil.

¹H NMR (400 MHz, CHLOROFORM-d) d ppm 5.05-4.92 (m, 2H), 2.66 (d, J=6.0 Hz, 4H), 2.59-2.53 (m, 4H), 2.40 (t, J=7.6 Hz, 4H), 2.32 (t, J=7.6 Hz, 4H), 1.72-1.53 (m, 18H), 1.43-1.28 (m, 46H), 0.90 (dt, J=2.8, 6.8 Hz, 12H).

### Procedure for Preparation of Compound 8

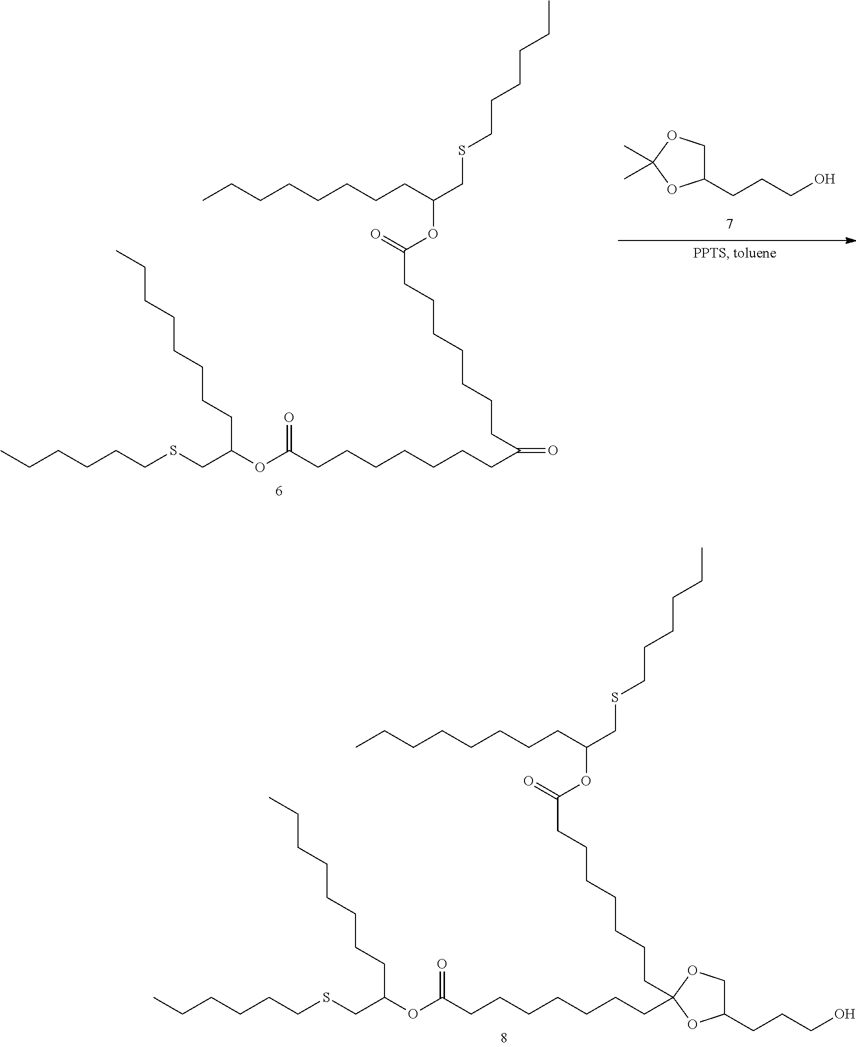 [SMILES: CCCCCCCCC(CSCCCCCC)OC(=O)CCCCCCCC(=O)CCCCCCCC(=O)OC(CCCCCCCC)CSCCCCCC ; CC1(C)OCC(CCCO)O1 ; CCCCCCCCC(CSCCCCCC)OC(=O)CCCCCCCC1(CCCCCCCC(=O)OC(CCCCCCCC)CSCCCCCC)OCC(CCCO)O1]

To a mixture of compound 6 (2.8 g, 3.38 mmol, 1 eq.) and compound 7 (1.36 g, 8.46 mmol, 2.5 eq.) in toluene (30 mL) was added PPTS (255.13 mg, 1.02 mmol, 0.3 eq.) at 25° C., and then the mixture was stirred at 130° C. for 12 hrs equipped with a Dean-Stark trap. The reaction mixture was poured into a suspension of NaHCO₃ and water (w/w=1/1, 50 mL) and stirred for 2 mins. The aqueous phase was extracted with DCM (40 mL*3). The combined organic phases were washed with brine (40 mL), dried over anhydrous Na₂SO₄, filtered and concentrated under vacuum. The residue was purified by column chromatography (SiO₂, Petroleum ether/Ethyl acetate=10/1 to 3/1) to give compound 8 (2.3 g, 72.39% yield) as a yellow oil.

¹H NMR (400 MHz, CHLOROFORM-d) δ ppm 5.07-4.91 (m, 2H), 4.19-4.06 (m, 2H), 3.80-3.67 (m, 2H), 3.50 (t, J=7.2 Hz, 1H), 2.67 (d, J=6.0 Hz, 4H), 2.57 (t, J=7.2 Hz, 4H), 2.33 (t, J=7.6 Hz, 4H), 1.61 (br dd, J=7.6, 14.2 Hz, 20H), 1.48-1.21 (m, 52H), 1.03-0.85 (m, 12H).

### Procedure for Preparation of Compound 9

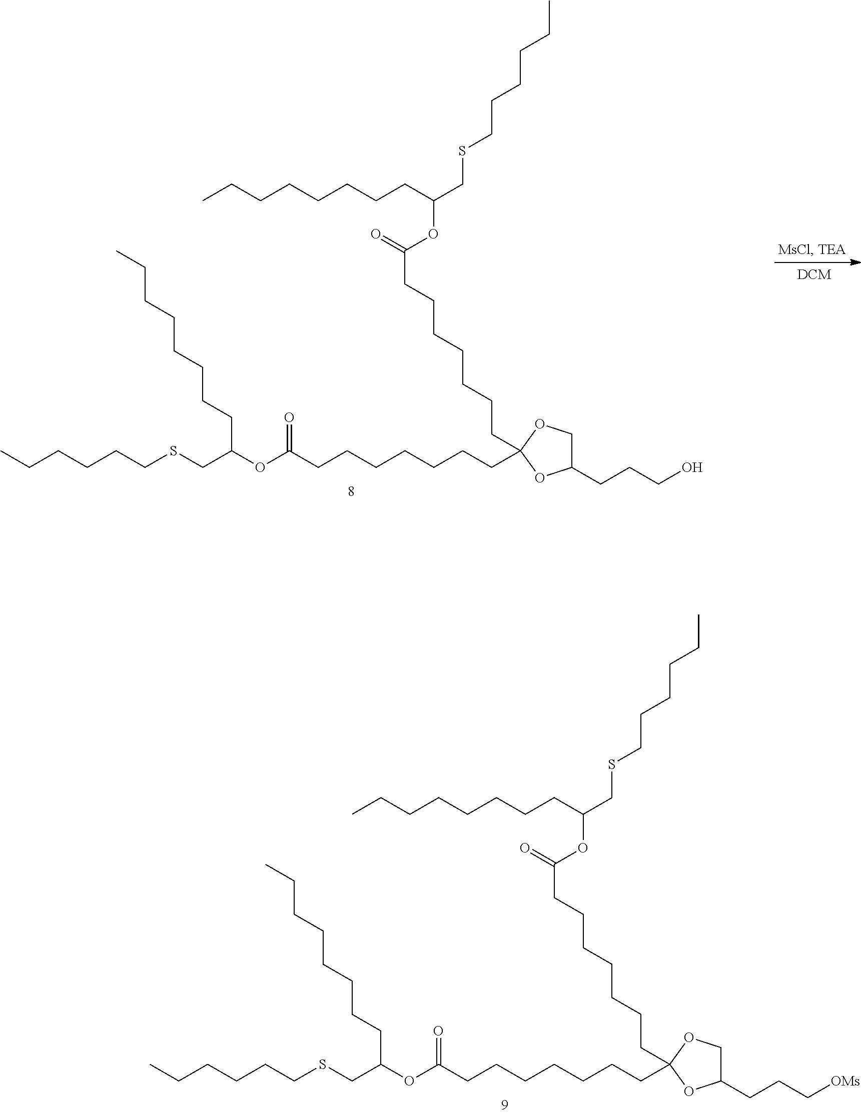 [SMILES: CCCCCCCCC(CSCCCCCC)OC(=O)CCCCCCCC1(CCCCCCCC(=O)OC(CCCCCCCC)CSCCCCCC)OCC(CCCO)O1 ; CCCCCCCCC(CSCCCCCC)OC(=O)CCCCCCCC1(CCCCCCCC(=O)OC(CCCCCCCC)CSCCCCCC)OCC(CCCOS(C)(=O)=O)O1]

To a solution of compound 8 (2.3 g, 2.47 mmol, 1 eq.) in DCM (20 mL) was added TEA (1.25 g, 12.37 mmol, 1.72 mL, 5 eq.) and MsCl (1.22 g, 10.64 mmol, 823.52 μL, 4.3 eq.) at 0° C., and then the mixture was stirred at 25° C. for 2 hrs. The reaction mixture was poured into ice-water (w/w=1/1, 50 mL) and stirred for 2 mins. The aqueous phase was extracted with DCM (40 mL*3). The combined organic phases were washed with brine (40 mL), dried over anhydrous Na₂SO₄, filtered and concentrated under vacuum. The residue was purified by column chromatography (SiO₂, Petroleum ether/Ethyl acetate=10/1 to 3/1) to give compound 9 (2.3 g, 92.25% yield) as a yellow oil.

¹H NMR (400 MHz, CHLOROFORM-d) δ ppm 5.06-4.91 (m, 2H), 4.40-4.25 (m, 2H), 4.13-4.04 (m, 2H), 3.57-3.44 (m, 1H), 3.04 (s, 3H), 2.67 (d, J=6.0 Hz, 4H), 2.57 (t, J=7.6 Hz, 4H), 2.33 (t, J=7.6 Hz, 4H), 2.00-1.81 (m, 2H), 1.78-1.61 (m, 12H), 1.59-1.53 (m, 6H), 1.45-1.26 (m, 52H), 0.91 (dt, J=2.8, 6.8 Hz, 12H).

### Procedure for Preparation of Lipid 54

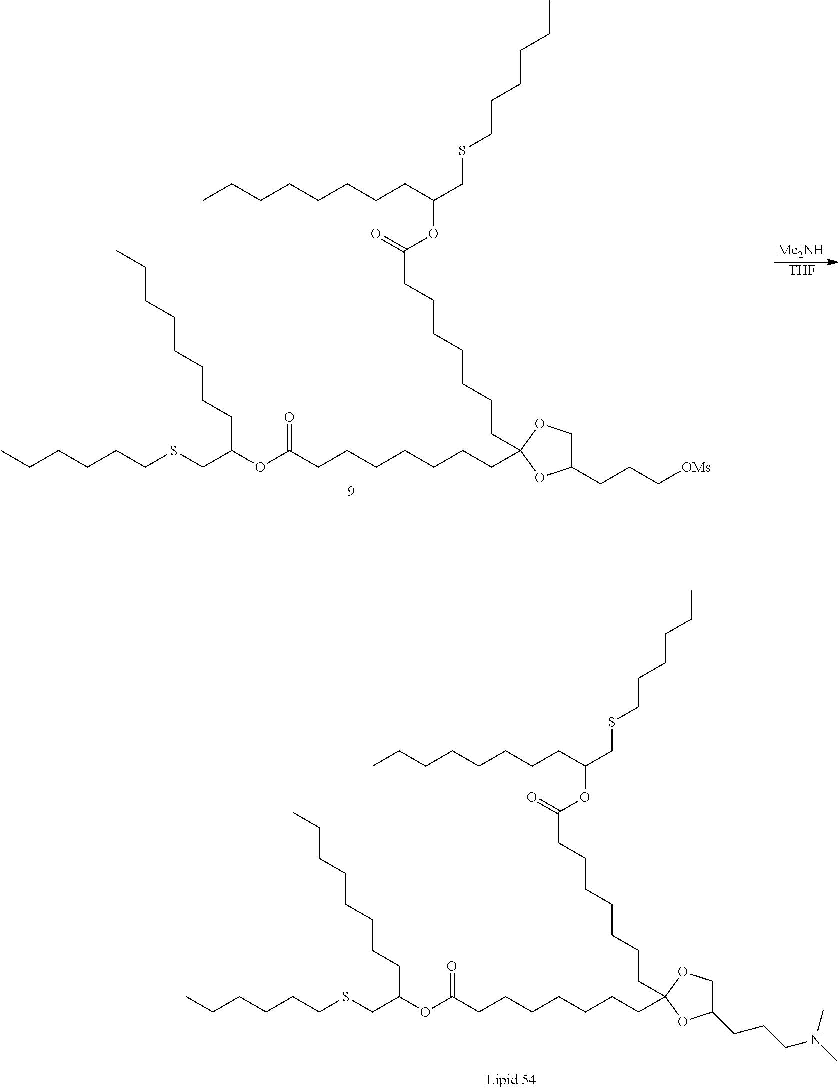 [SMILES: "Me2NH" ; CCCCCCCCC(CSCCCCCC)OC(=O)CCCCCCCC1(CCCCCCCC(=O)OC(CCCCCCCC)CSCCCCCC)OCC(CCCOS(C)(=O)=O)O1 ; CCCCCCCCC(CSCCCCCC)OC(=O)CCCCCCCC1(CCCCCCCC(=O)OC(CCCCCCCC)CSCCCCCC)OCC(CCCN(C)C)O1 ; "THF"]

To a solution of compound 9 (2.3 g, 2.28 mmol, 1 eq.) in THF (20 mL) was added Me₂NH (2 M THF solution, 20 mL) at 25° C., and then the mixture was stirred at 40° C. for 12 hrs. The reaction mixture was concentrated under reduced pressure. The residue was purified by column chromatography (SiO₂, Petroleum ether/Ethyl acetate with 0.1% TEA=10/1 to 3/1), followed by prep-NPLC (column: Welch Ultimate XB—NH₂ 250*50 mm*10 μm; mobile phase: [Hexane-EtOH]; B %: 1%, isocratic elution mode) to give Lipid 54 (400 mg, 17.67% yield) as a light-yellow oil.

HRMS: m/z 956.7813 [M+1]⁺; ¹H NMR (400 MHz, CHLOROFORM-d) δ ppm 5.06-4.92 (m, 2H), 4.17-4.02 (m, 2H), 3.55-3.43 (m, 1H), 2.67 (d, J=6.0 Hz, 4H), 2.57 (t, J=7.6 Hz, 4H), 2.51-2.29 (m, 11H), 1.73-1.54 (m, 22H), 1.48-1.23 (m, 52H), 0.91 (dt, J=2.8, 6.8 Hz, 12H).

### Synthesis of Lipid 55

### Procedure for Preparation of Compound 2

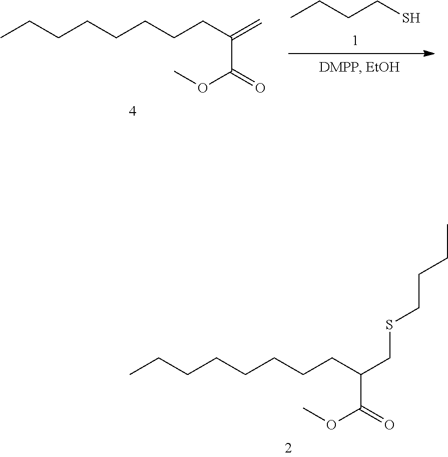

To a solution of compound 4 (250 mg, 1.26 mmol, 1 eq.) in EtOH (4.00 mL) was added DMPP (34.8 mg, 252. μmol, 0.2 eq) and compound 1 (171 mg, 1.89 mmol, 1.5 eq.) at 25° C., and then the mixture was stirred at 80° C. for 2 hrs under N₂ atmosphere. The reaction mixture was concentrated under reduced pressure. The residue was purified by column chromatography (SiO₂, PE: Ethyl acetate=I/O to 20/1) to give compound 2 (344 mg, 85% yield) as a colorless liquid.

LCMS: m/z 289.1 [M+1]⁺; ¹H NMR (400 MHz, CHLOROFORM-d) δ ppm 3.71 (s, 2H), 2.72-2.80 (m, 1H), 2.56-2.67 (m, 2H), 2.52 (t, J=7.4 Hz, 2H), 1.53-1.64 (m, 4H), 1.39-1.46 (m, 2H), 1.23-1.31 (m, 12H), 0.84-0.96 (m, 6H).

### Procedure for Preparation of Compound 3

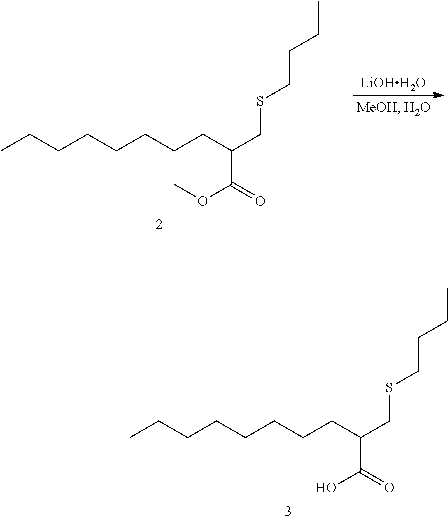

To a solution of compound 2 (344 mg, 1.19 mmol, 1 eq.) in a mixed solvent of MeOH (2.5 mL) and H₂O (0.5 mL) was added LiOH·H₂O (150 mg, 3.58 mmol, 3 eq.) at 25° C., and then the mixture was stirred at 50° C. for 12 hrs. The reaction mixture was quenched by addition of 1N HCl (20 mL) at 0° C., and then diluted with water (10 mL) and extracted with EtOAc (20 mL*3). The combined organic layers were washed with brine (20 mL*2), dried over anhydrous Na₂SO₄, filtered and concentrated under reduced pressure. The residue was purified by column chromatography (SiO₂, PE: Ethyl acetate=I/O to 10/1) to give compound 4 (252 mg, 69. % yield) as a yellow oil.

LCMS: m/z 275.2 [M+1]⁺; ¹H NMR (400 MHz, CHLOROFORM-d) δ ppm 2.76-2.84 (m, 1H), 2.59-2.69 (m, 2H), 2.54 (t, J=7.2 Hz, 2H), 1.52-1.70 (m, 4H), 1.41 (dd, J=15.2, 7.2 Hz, 2H), 1.27 (m, 12H), 0.87-0.94 (m, 6H).

### Procedure for Preparation of Lipid 55

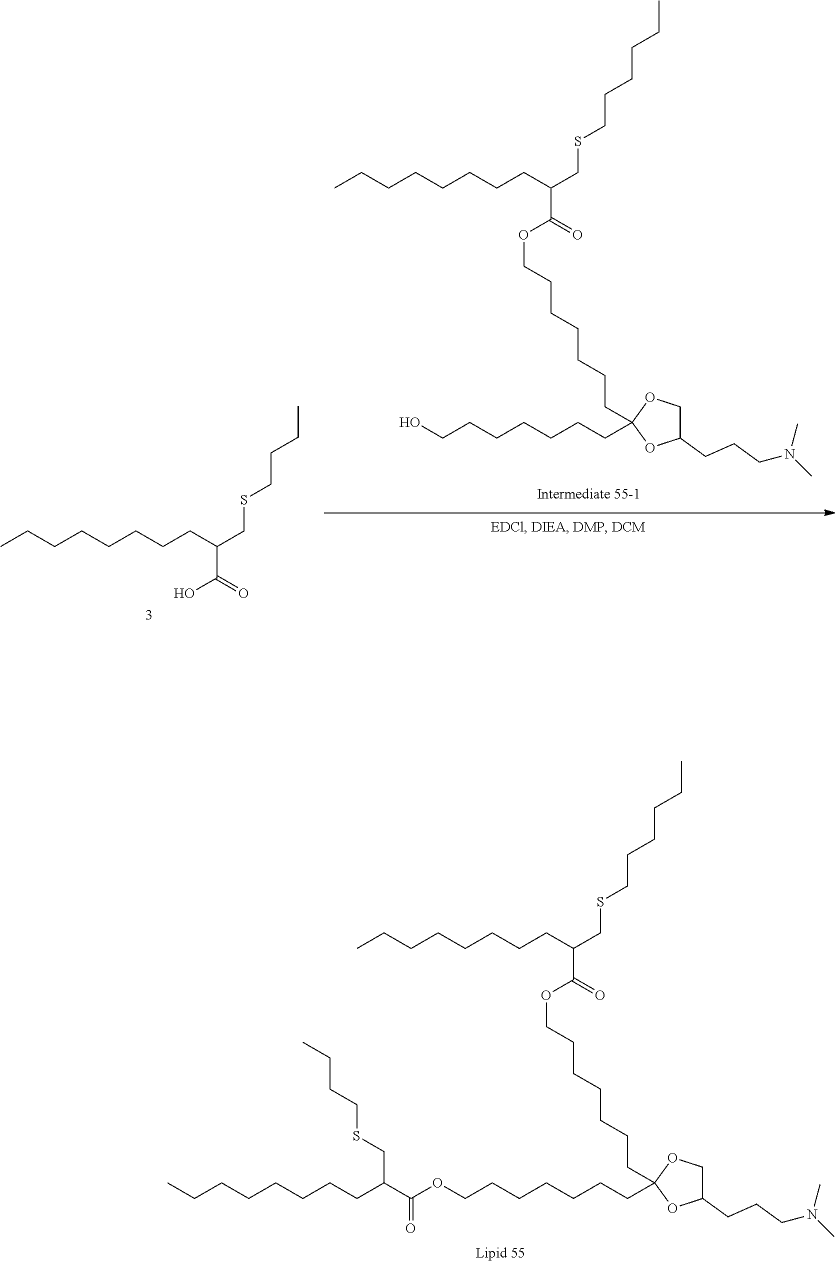 [SMILES: CCCCCCCCC(CSCCCC)C(=O)O ; CCCCCCCCC(CSCCCCCC)C(=O)OCCCCCCCC1(CCCCCCCO)OCC(CCCN(C)C)O1 ; CCCCCCCCC(CSCCCC)C(=O)OCCCCCCCC1(CCCCCCCOC(=O)C(CCCCCCCC)CSCCCCCC)OCC(CCCN(C)C)O1]

To a mixture of compound 3 (200 mg, 729 μmol, 1 eq.) and intermediate 55-1 (490 mg, 729 μmol, 1 eq.) in DCM (4 mL) was added DMAP (97.9 mg, 802 μmol, 1.1 eq.), DIEA (188 mg, 1.46 mmol, 2 eq.) and EDCI (279 mg, 1.46 mmol, 2 eq.), and then the mixture was stirred at 25° C. for 12 hrs. The reaction mixture was concentrated under reduced pressure. The residue was purified by flash silica gel chromatography (ISCO®; 20 g SepaFlash® Silica Flash Column, Eluent of 0˜10% PE/EA gradient @ 40 mL/min), followed by prep-HPLC (column: X-Select CSH Phenyl-Hexyl 100*30 5u; mobile phase: [H₂O (0.225% FA)-EtOH]; gradient: 68%-98% B over 25.0 min) to give Lipid 55 (217.9 mg, 31.16% yield) as a yellow oil.

HRMS: m/z 928.7453 [M+1]⁺; ¹H NMR (400 MHz, CHLOROFORM-d) δ ppm 4.00-4.14 (m, 6H), 3.41-3.50 (m, 1H), 2.71-2.81 (m, 2H), 2.48-2.65 (m, 8H), 2.30 (t, J=6.8 Hz, 2H), 2.23 (s, 6H), 1.53-1.67 (m, 18H), 1.19-1.43 (m, 50H), 0.85-0.94 (m, 12H).

### Synthesis of Lipid 56

### Procedure for Preparation of Compound 2

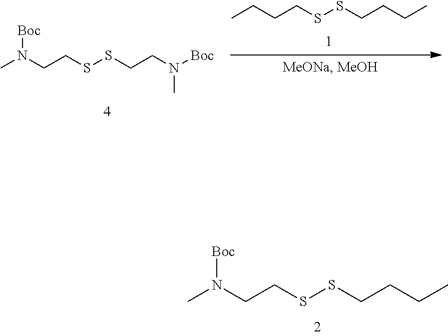

To a mixture of compound 4 (1 g, 2.63 mmol, 1 eq.) and compound 1 (468.67 mg, 2.63 mmol, 1 eq.) in MeOH (10 mL) was added NaOMe (5.4 M MeOH solution, 5.00 mL), and then the mixture was stirred at 25° C. for 12 hrs. The reaction mixture was concentrated under reduced pressure. The residue was purified by column chromatography (SiO₂, PE:EA=I/O to 3/1) to give compound 2 (700 mg) as a yellow oil.

### Procedure for Preparation of Compound 3

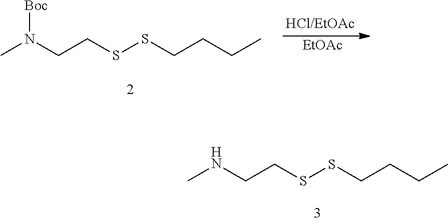

To a mixture of compound 2 (600 mg, crude) in EtOAc (50 mL) was added HCl/EtOAc (4 M, 11.04 mL), and then the mixture was stirred at 25° C. for 2 hrs. The reaction mixture was concentrated under reduced pressure to give crude compound 3 (375 mg, 76.89% yield, HCl salt) as a white solid.

¹H NMR (400 MHz, DMSO-d₆) δ ppm 9.16 (br s, 2H), 3.30 (s, 3H), 3.14-3.05 (m, 2H), 3.00-2.92 (m, 2H), 2.71 (t, J=7.2 Hz, 2H), 1.57 (quin, J=7.3 Hz, 2H), 1.33 (sxt, J=7.2 Hz, 2H), 0.85 (t, J=7.2 Hz, 3H).

### Procedure for Preparation of Lipid 56

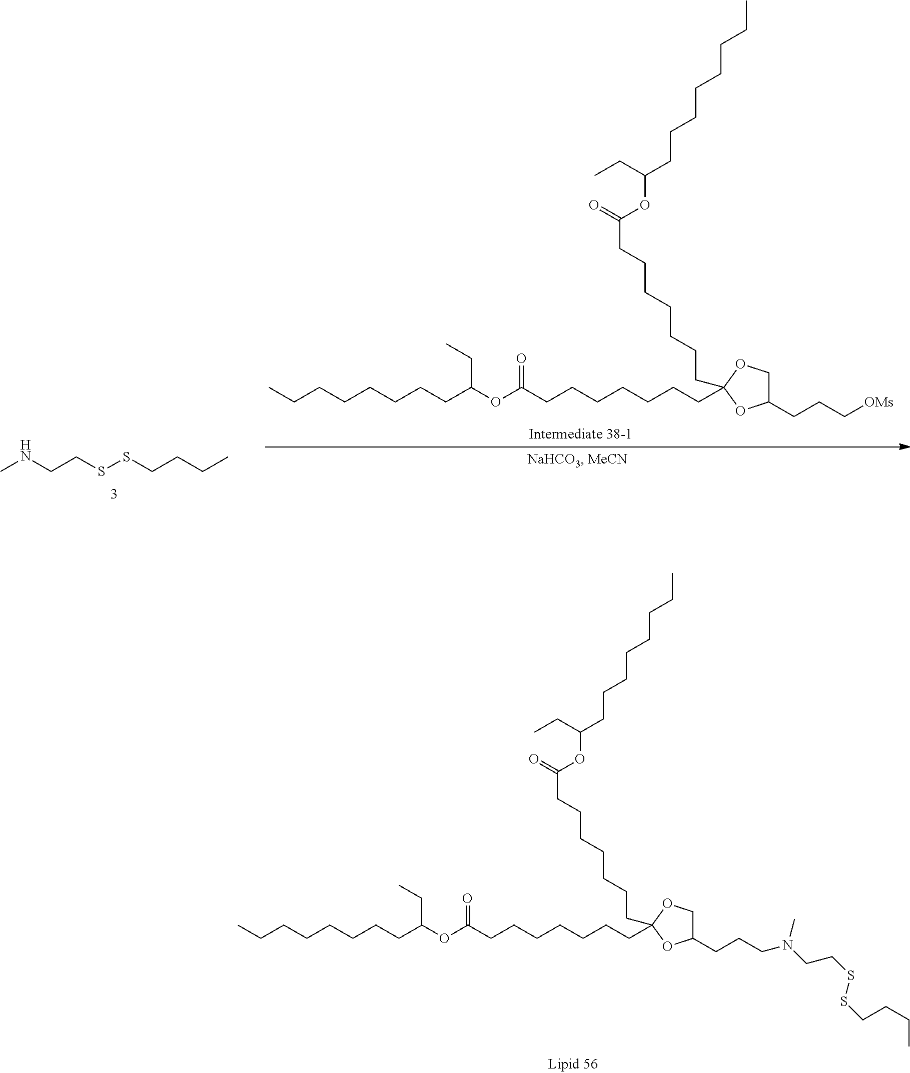

To a mixture of intermediate 38-1 (400 mg, 506.85 μmol, 1 eq.) and compound 3 (328.14 mg, 1.52 mmol, 3 eq., HCl salt) in MeCN (5 mL) was added NaHCO₃ (212.89 mg, 2.53 mmol, 5 eq.), and then the mixture was stirred at 85° C. for 16 hrs. The reaction mixture was filtered and the filtrate was concentrated under reduced pressure. The residue was purified by column chromatography (SiO₂, PE/EA=10/1 to 5/1, eluting with 0.1% TEA), followed by column purification (SiO₂, A/B=100/1 to 5/1, phase A: PE:DCM=3:1, phase B: EtOAc, eluting with 0.1% TEA) to give Lipid 56 (230 mg, 38.33% yield) as a colourless oil.

HRMS: m/z 886.7016 [M+1]⁺; ¹H NMR (400 MHz, CHLOROFORM-d) δ ppm 4.82 (quin, J=6.4 Hz, 2H), 4.11-3.98 (m, 2H), 3.51-3.40 (m, 1H), 2.86-2.75 (m, 2H), 2.71 (s, 4H), 2.41 (br t, J=6.0 Hz, 2H), 2.33-2.22 (m, 7H), 1.72-1.48 (m, 22H), 1.46-1.39 (m, 2H), 1.38-1.21 (m, 40H), 0.98-0.84 (m, 15H).

### Example 2. Preparation of LNPS

Exemplary LNPs were produced using single ionizable Lipid 1 as synthesized in Example 1.

### Preparation of LNP 1

A lipid solution for LNP 1 was prepared according to Table 1A and the following:
1) The lipid solution was prepared in BSC hood to ensure clean environment.
2) Autoclaved 4 mL glass vials and RNAse/DNAse-free conical tubes were used for lipid solution and RNA solution preparation, respectively.
3) All buffer used in preparation was filtered through 0.2 μm membrane.
4) The lipid solutions were prepared by mixing individual lipid stock solutions which were filtered through 0.2 μm PTFE syringe filters.
5) The RNA solution were prepared by mixing the RNA stock solutions with buffer which was filtered through filter units with 0.2 μm membrane.
6) All prepared solutions were used within 1 hour for LNP fabrications.

TABLE 1A

| | Concentration of lipid | Volume of lipid | Lipid |
|---|---|---|---|
| | stock solution in | stock solution | molar |
| Component | ethanol (mM) | (μL) | ratio |
| Lipid 1 | 20 | 450 | 49.22 |
| Cholesterol | 45 | 160 | 39.37 |
| DSPC | 20 | 90 | 9.84 |
| DMG-PEG2K | 20 | 13.8 | 1.51 |
| DiIC18(5)-DS | 0.5 | 21.9 | 0.06 |
| Ethanol | | 1264.3 | |
| Total lipid solution | | 2000 | |

### Preparation of LNP 2

A lipid solution for LNP 2 was prepared according to Table 2A and the following:
1) The lipid solution was prepared in BSC hood to ensure clean environment.
2) Autoclaved 4 mL glass vials and RNAse/DNAse-free conical tubes were used for lipid solution and DNA solution preparation, respectively.
3) All buffer used in preparation was filtered through 0.2 μm membrane.
4) The lipid solutions were prepared by mixing individual lipid stock solutions which were filtered through 0.2 μm PTFE syringe filters.
5) The DNA solutions were prepared by mixing the DNA stock solution with buffer which was filtered through filter units with 0.2 μm membrane.
6) All prepared solutions were used within 1 hour for LNP fabrications.

TABLE 2A

| | | Lipid stock | Lipid |
|---|---|---|---|
| | Lipid stock solution | solution vol. | molar |
| Component | concentration (mM) | (μL) | ratio |
| Lipid 1 | 20 | 71.2 | 49.22 |
| Cholesterol | 20 | 57 | 39.37 |
| DSPC | 10 | 28.5 | 9.84 |
| DMG-PEG2K | 2 | 19.0 | 1.31 |
| Tri-GalNAc | 0.5 | 11.6 | 0.20 |
| DiIC18(5)-DS | 0.5 | 8.7 | 0.06 |
| Ethanol | | 404.2 | |
| Total lipid solution | | 600 | |

LNP 2 formulation was prepared according to Table 2B and the following:
1) 1.40 mL of DNA solution and 0.467 mL of lipid solution were loaded in a 3 mL BD syringe and a 1 mL BD syringe, respectively.
2) The syringes were loaded on Ignite with the following parameters: N/P ratio=4.81, flow ratio=3, start waste=0.45 mL, end waste=0.05 mL.
3) A 15 mL Falcon tube was loaded in the sample position and Ignite was run.
4) 1.37 mL of LNPs were collected and immediately diluted by adding 26.15 mL of HEPES buffered saline (25 mM HEPES, 150 mM NaCl, pH 7.4).
5) The diluted LNPs sample was transferred to one well of 24-well plate (100,000 kDa MWCOmembrane with volume of 10 ml/well) and loaded on Big Tuna for buffer exchange and concentration.
6) HEPES buffered saline was loaded in reservoir. Buffer exchange target was set as 95%.
7) After buffer exchange, the LNP sample was concentrated to 500 μL and collected into 1.5 mL Eppendorf tube.
8) The collected sample was filtered through 0.2 μm syringe filter and stored in 4° C. fridge.

TABLE 2B

| Component | Vol (μL) |
|---|---|
| 1 mg/mL ceNLuci-mCherry stock | 75 |
| Buffer (20 mM Malic acid, 30 mM NaCl, pH 3.0) | 1425 |
| Total DNA solution | 1500 |

### Preparation of LNP 3

A lipid solution for LNP 3 was prepared according to Table 3A and the following:
1) The lipid solution was prepared in BSC hood to ensure clean environment.
2) Autoclaved 4 mL glass vials and RNAse/DNAse-free conical tubes were used for lipid solution and DNA solution preparation, respectively.
3) All buffer used in preparation was filtered through 0.2 μm membrane.
4) The lipid solutions were prepared by mixing individual lipid stock solutions which were filtered through 0.2 μm PTFE syringe filters.
5) The DNA solutions were prepared by mixing the DNA stock solution with buffer which was filtered through filter units with 0.2 μm membrane.
6) All prepared solutions were used within 1 hour for LNP fabrications.

TABLE 3A

| | Lipid stock | Lipid stock | Lipid |
|---|---|---|---|
| | solution | solution | molar |
| Component | concentration (mM) | volume (μL) | ratio |
| Lipid 1 | 20 | 474.7 | 49.22 |
| Cholesterol | 45 | 168.7 | 39.37 |
| DSPC | 20 | 95.5 | 9.90 |
| DMG-PEG2K | 20 | 12.6 | 1.31 |
| Tri-GalNAc | 0.5 | 77.2 | 0.20 |
| Ethanol | | 3171.3 | |
| Total lipid solution | | 4000 | |

LNP 3 formulation was prepared according to Table 3B and the following:
1) 9.90 mL of DNA solution and 3.30 mL of lipid solution were loaded in a 10 mL BD syringe and a 5 mL BD syringe, respectively.
2) The syringes were loaded on Ignite with the following parameters: N/P ratio=4.81, flow ratio=3, start waste=1.00 mL, end waste=0.05 mL.
3) A 15 mL Falcon tube was loaded in the sample position and Ignite was run.
4) 12.15 mL of LNPs were collected and immediately diluted by adding 109.35 mL of HEPES buffered saline (25 mM HEPES, 150 mM NaCl, pH 7.4).
5) The diluted LNPs sample was concentrated to ˜30 mL using Repligen KrosFlo KR2i TFF system, and buffer exchanged to HEPES buffered saline.
6) After buffer exchange, the LNP sample was concentrated to ˜12 mL and collected into a 50 mL Falcon tube.
7) The LNP sample was further concentrated to 1.0 ml using Repligen MicroKros 100,000 kDa MWCO Hollowfiber, and then filtered through 0.2 μm syringe filter and stored in 4° C. fridge.

TABLE 3B

| Component | Vol (μL) |
|---|---|
| 1 mg/mL ceNLuci-mCherry stock (μL) | 500 |
| Buffer (20 mM Malic acid, 30 mM NaCl, pH 3.0) | 9500 |
| Total DNA solution | 10000 |

### Preparation of LNP 4

A lipid solution for LNP 4 was prepared according to Table 4A and the following:
1) The lipid solution was prepared in BSC hood to ensure clean environment.
2) Autoclaved 4 mL glass vials and RNAse/DNAse-free conical tubes were used for lipid solution and DNA solution preparation, respectively.
3) All buffer used in preparation was filtered through 0.2 μm membrane.
4) The lipid solutions were prepared by mixing individual lipid stock solutions which were filtered through 0.2 μm PTFE syringe filters.
5) The Nanoplasmid DNA solutions were prepared by mixing the DNA stock solution with buffer which was filtered through filter units with 0.2 μm membrane.
6) All prepared solutions were used within 1 hour for LNP fabrications.

TABLE 4A

| | Lipid stock | Lipid stock | Lipid |
|---|---|---|---|
| | solution | solution | molar |
| Component | concentration (mM) | volume (μL) | ratio |
| 15b | 10 | 29.6 | 5.29 |
| Lipid 1 | 20 | 125.1 | 44.71 |
| Cholesterol | 20 | 105 | 37.50 |
| DSPC | 10 | 56 | 10.00 |
| DMG-PEG2K | 2 | 70 | 2.50 |
| Ethanol | | 414.4 | |
| Total lipid solution | | 800 | |

*15b is a lipid with the following structure:

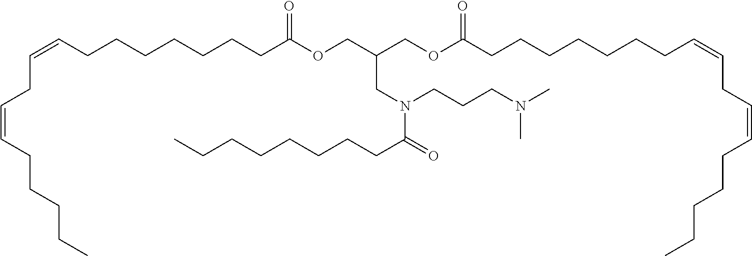 [SMILES: CCCCC/C=C\C/C=C\CCCCCCCC(=O)OCC(COC(=O)CCCCCCC/C=C\C/C=C\CCCCC)CN(CCCN(C)C)C(=O)CCCCCCCC]

LNP 4 formulation was prepared according to Table 4B and the following:
1) 1.90 mL of DNA solution and 0.633 mL of lipid solution were loaded in a 3 mL BD syringe and a 1 mL BD syringe, respectively.
2) The syringes were loaded on Ignite with the following parameters: N (15b)/P ratio=0.75, N (Lipid 1)/P ratio=6.34, flow ratio=3, start waste=0.45 mL, end waste=0.05 mL.
3) A 15 mL Falcon tube was loaded in the sample position and Ignite was run.
4) 2.03 mL of LNPs were collected and immediately diluted by adding 10.17 mL of HEPES buffered saline (25 mM HEPES, 150 mM NaCl, pH 7.4).
5) The diluted LNPs sample was transferred to two wells of 24-well plate (100,000 kDa MWCOmembrane with volume of 10 ml/well) and loaded on Big Tuna for buffer exchange and concentration.
6) HEPES buffered saline was loaded in reservoir. Buffer exchange target was set as 95%.
7) After buffer exchange, the LNP sample was concentrated to 500 μL and collected into 1.5 mL Eppendorf tube.
8) The collected sample was filtered through 0.2 μm syringe filter and stored in 4° C. fridge.

TABLE 4B

| Component | Vol (μL) |
|---|---|
| 1 mg/mL NLuci-mCherry stock (μL) | 100 |
| Buffer (20 mM Malic acid, 30 mM NaCl, pH 3.0) | 1900 |
| Total DNA solution | 2000 |

### Example 3. Characterization of LNPS

This Example describes the characterization of LNPs produced in Example 2. Samples of the LNPs produced in Example 2 were characterized to determine the average hydrodynamic diameter, polydispersity index (PDI), zeta potential, and encapsulation efficiency. The results are set forth in Table 5.

TABLE 5

| | | | Zeta | | Zeta | Encapsulation |
|---|---|---|---|---|---|---|
| | Components of LNP | | Average | | potential | efficiency |
| LNP | (molar ratio) | Payload | (nm) | PDI | (mV) | (%) |
| 1 | Lipid 1:Chol:DSPC:DMG- | RNA | 79 | 0.06 | 1.5 | 59 |
| | PEG2000:DilC18(5)-DS = | | | | | |
| | 49.22:39.37:9.84:1.51:0.06 | | | | | |
| 2 | Lipid 1:Chol:DSPC:DMG- | FLuci-mCherry, | 114 | 0.2 | −0.2 | 93 |
| | PEG2000:Tri- | V.2 | | | | |
| | GalNA:DilC18(5)-DS = | | | | | |
| | 49.22:39.37:9.84:1.31:0.2:0.06 | | | | | |
| 3 | Lipid 1:Chol:DSPC:DMG- | FLuci-mCherry, | 112 | 0.06 | −3.7 | 85 |
| | PEG2000:Tri-GalNA = | V.2 | | | | |
| | 49.22:39.37:9.9:1.31:0.2 | | | | | |
| 4 | 15b:Lipid 1:Chol:DSPC:DPG- | Luci | 98 | 0.15 | 1.4 | 94 |
| | PEG2000 = | Nanoplasmid | | | | |
| | 5.29:44.71:37.5:10:2.5 | DNA | | | | |

*Chol = Cholesterol; DSPC = 1,2-Distearoyl-sn-glycero-3-phosphorylcholine; DMG-PEG2000 = 1,2-dimyristoyl-rac-glycero-3-methoxypolyethylene glycol-2000; DilC18(5)-DS = 1,1-Dioctadecyl-3,3,3,3-tetramethylindodicarbocyanine-5,5-disulfonic acid.

### Example 4. LNP Stabilities after Freeze/Thaw Cycle

### 1. Preparation of LNPS

A lipid solution for an LNP using an ionizable lipid of the present disclosure was prepared according to Table 6 and the following:
1. The lipid solution was prepared in BSC hood to ensure clean environment.
2. 4 mL glass vials were autoclaved and RNAse/DNAse-free conical tubes were used for lipid solution and mRNA solution preparation, respectively.
3. All buffer used in preparation was filtered through 0.2 μm membrane.
4. The lipid solutions were prepared by mixing individual lipid stock solutions which were filtered through 0.2 μm PTFE syringe filters.
5. The mRNA solution was prepared by mixing the mRNA stock solution with desired buffer which was filtered thorough filter units with 0.2 μm membrane.
6. All prepared solutions were used within 1 hour for LNP fabrications.

TABLE 6
Preparation of lipid solution

| | Conc. of | Vol of | |
|---|---|---|---|
| | lipid stock | lipid stock | Lipid |
| | solution in | solution | molar |
| Component | ethanol (mM) | (μL) | ratio |
| Ionizable lipid | 50 | 36.0 | 49.22 |
| Chol | 45 | 32.0 | 39.38 |
| DSPC | 20 | 18.1 | 9.9 |
| DMG-PEG2K | 2 | 27.4 | 1.5 |
| Ethanol | — | 286.5 | — |
| Total lipid | — | 400 | — |
| solution | | | |

Note:
the molar ratio between amines (N) in ionizable lipid and the phosphate (P) in the mRNA backbone (N/P molar ratio) = 4.81; Chol = Cholesterol; DSPC = 1,2-Distearoyl-sn-glycero-3-phosphorylcholine; DMG-PEG2000 = 1,2-dimyristoyl-rac-glycero-3-methoxypolyethylene glycol-2000.

### 2. Preparation of Firefly Luciferase (FLuc) MRNA LNP Formulations

Firefly luciferace (FLuc) mRNA LNP formulation baed on an LNP of the present disclosure is prepared according to Table 7 and below:
1. 0.9 mL of RNA solution and 0.3 mL of lipid solution were loaded in two 1 mL BD syringes, respectively.
2. The syringes were loaded on Ignite with the desired parameters.
3. A 15 mL Falcon tube was loaded in the sample position and run Ignite with flow rate of 20 mL/min and flow ratio of 3:1.
4. 0.85 mL of LNPs were collected and immediately diluted by adding 3.4 mL of HEPES buffered saline (25 mM HEPES, 75 mM NaCl, 5% sucrose, pH 7.4).
5. The diluted LNPs sample was transferred to a 50 mL Amicon tubes with 100,000 kDa MWCO filter membrane for buffer exchange and concentration.
6. The Amicon tube with LNP sample was centrifuged @ 500 rcf until volume was decreased to 1 mL.
7. 1 mL of HEPES buffered saline was added and centrifuged until volume was decreased to 1 mL. Repeated once.
8. 1 mL of HEPES buffered saline was added and centrifuged until volume was decreased to 0.5 mL.
9. The sample was collected and filtered through 0.2 μm syringe filter with PES membrane.
10. The filtered sample was diluted to 100 μg/mL, and stored in 2 mL Eppendorf tube in 4 C fridge.

TABLE 7
Preparation of Firefly Luciferase (FLuc) mRNA solution

| Component | Vol (uL) |
|---|---|
| 1 mg/mL FLuc mRNA stock (uL) | 100 |
| Buffer (30 mM Sodium Acetate, 30 mM NaCl, pH 4.0) | 900 |
| Total FLuc mRNA solution | 1000 |

3. Characterization Data and Freeze/Thaw (F/T) Stability of FLuc mRNA LNP Formulations

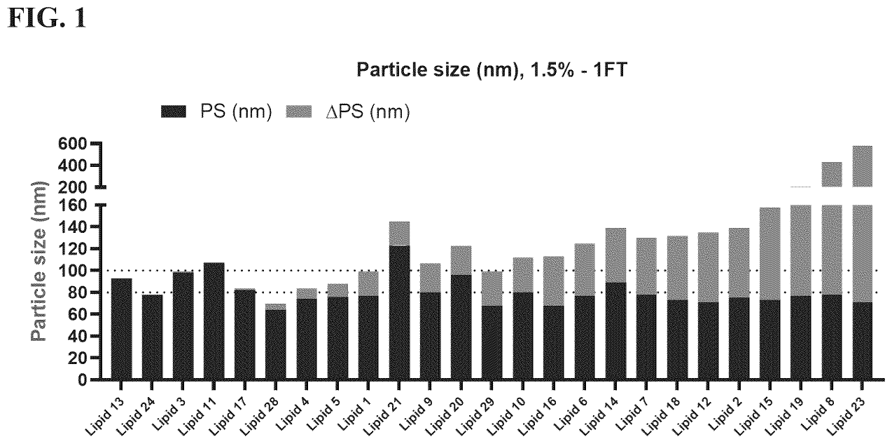

LNPs prepared were characterized to determine their pKa, PS, pDI, ZP, EE % before and after one Freeze/Thaw (F/T) cycle. The results are summarized in Table 8 and FIG. 1.

TABLE 8
Characterizations of FLuc mRNA LNP
formulations pre- and post-F/T

| | | PS | | ZP | EE | Post 1 F/T cycle | | |
|---|---|---|---|---|---|---|---|---|
| Lipids | pKa | (nm) | PdI | (mV) | % | PS (nm) | PdI | ZP (mV) |
| Lipid 13 | 6.703 | 93 | 0.2 | −2.4 | 89 | 89 | 0.08 | −1.3 |
| Lipid 24 | 5.17 | 78 | 0.04 | −15.4 | 66 | 78 | 0.05 | −18.3 |
| Lipid 3 | 6.81 | 98 | 0.22 | −10.6 | 95 | 99 | 0.08 | −5.2 |
| Lipid 11 | 6.60 | 107 | 0.01 | 0 | 95 | 108 | 0.02 | −6.1 |
| Lipid 17 | 6.57 | 82 | 0.11 | −6.6 | 93 | 84 | 0.1 | −8 |
| Lipid 28 | 7.23 | 64 | 0.14 | 10.4 | 91 | 70 | 0.19 | 6.6 |
| Lipid 4 | 6.76 | 74 | 0.08 | −6.8 | 97 | 84 | 0.06 | −12 |
| Lipid 5 | 6.51 | 76 | 0.09 | −12.6 | 95 | 88 | 0.08 | −8.7 |
| Lipid 1 | 7.13 | 77 | 0.07 | 1.4 | 95 | 99 | 0.14 | 0.6 |
| Lipid 21 | 6.91 | 123 | 0.02 | −1.9 | 82 | 145 | 0.04 | −4 |
| Lipid 9 | 6.85 | 80 | 0.06 | −5.6 | 96 | 107 | 0.06 | −3.4 |
| Lipid 20 | 7.31 | 96 | 0.04 | 5.9 | 94 | 123 | 0.12 | 6 |
| Lipid 30 | 7.68 | 68 | 0.11 | 9.4 | 90 | 99 | 0.09 | 0.2 |
| Lipid 10 | 7.38 | 80 | 0.1 | −7.5 | 93 | 112 | 0.22 | −6.2 |
| Lipid 16 | 7.44 | 68 | 0.05 | 2.5 | 96 | 113 | 0.31 | 5.4 |
| Lipid 6 | 7.28 | 77 | 0.18 | 2 | 96 | 125 | 0.16 | 7.1 |
| Lipid 14 | 6.66 | 89 | 0.05 | −2.9 | 72 | 139 | 0.13 | −4.2 |
| Lipid 7 | 7.54 | 78 | 0.13 | 0.8 | 94 | 130 | 0.14 | 1.1 |
| Lipid 18 | 7.26 | 73 | 0.05 | −0.3 | 95 | 132 | 0.14 | 0.2 |
| Lipid 12 | 7.38 | 71 | 0.09 | 2.6 | 94 | 135 | 0.14 | 0.1 |
| Lipid 2 | 7.04 | 75 | 0.03 | −0.7 | 94 | 139 | 0.11 | 0.5 |
| Lipid 15 | 7.04 | 73 | 0.03 | −1.2 | 96 | 158 | 0.17 | 1.8 |
| Lipid 19 | 6.88 | 77 | 0.11 | −2.1 | 87 | 210 | 0.1 | −2 |
| Lipid 8 | 7.03 | 78 | 0.05 | −3.6 | 87 | 432 | 0.49 | 2.7 |
| Lipid 23 | 7.45 | 71 | 0.12 | 0 | 84 | 583 | 0.65 | −4.8 |

Note:
Note:
Molar ratio of ionizable lipid:cholesterol:DSPC:DMG-PEG2K = 49.22:39.38:9.9:1.5; N/P molar ratio = 4.81; PS = Particle size; PDI = Polydispersity; ZP = Zeta potential; EE = Encapsulation efficiency; F/T = Freeze/Thaw

As shown in FIG. 1, LNPs made of Lipid 13, Lipid 24, Lipid 3, Lipid 11, and Lipid 17 are all very stable over one freeze-thaw cycle.

### Example 5. Modifying Surface Charge of Lipid 24 Lnps by Varying Formulation N/P Ratio

### 1. Preparation of LNP6

A lipid solution for an LNP using Lipid 24 was prepared according to Table 9 and the following:
1. The lipid solution was prepared in BSC hood to ensure clean environment.
2. Autoclaved 4 mL glass vials and RNAse/DNAse-free conical tubes were used for lipid solution and DNA solution preparation, respectively.
3. All buffer used in preparation was filtered through 0.2 μm membrane.
4. The lipid solutions were prepared by mixing individual lipid stock solutions which were filtered through 0.2 μm PTFE syringe filters.
5. The DNA solution was prepared by mixing the DNA stock solution with desired buffer which was filtered thorough filter units with 0.2 μm membrane.
6. All prepared solutions were used within 1 hours for LNP fabrications.

TABLE 9
Preparation of lipid solution (LNP 6)

| | Conc. of | Vol of | |
|---|---|---|---|
| | lipid stock | lipid stock | Lipid |
| | solution in | solution | molar |
| Component | ethanol (mM) | (uL) | ratio |
| LNP 24 | 20 | 59.2 | 49.22 |
| Chol | 45 | 21.0 | 39.37 |
| DSPC | 20 | 11.9 | 9.90 |
| DMG-PEG2K | 20 | 1.2 | 1.01 |
| Tri-GalNAc | 0.5 | 24.1 | 0.5 |
| Ethanol | — | 282.6 | — |
| Total lipid solution | — | 400 | — |

Note:
N/P molar ratio = 6; Tri-GalNAc = Triantennary N-acetyl galactosamine.

2. Preparation of Firefly Luciferase (FLuc) mRNA LNP Formulations (LNP 6)

A lipid solution for an LNP using Lipid 24 was prepared according to Table 10 and the following:
1. 0.9 mL of DNA solution and 0.3 mL of lipid solution were loaded in two 1 mL BD syringes, respectively.
2. The syringes were loaded on Ignite with the desired parameters.
3. A 15 mL Falcon tube was loaded in the sample position and run Ignite with flow rate of 20 mL/min and flow ratio of 3:1.
4. 0.85 mL of LNPs were collected and immediately diluted by adding 3.4 mL of 1×HEPES buffered saline (25 mM HEPES, 150 mM NaCl, pH7.4).
5. The diluted LNPs sample was transferred to a 50 mL Amicon tubes with 100,000 kDa MWCO filter membrane for buffer exchange and concentration.
6. The Amicon tube with LNP sample was centrifuged @ 500rcf until volume was decreased to 1 mL.
7. 1 mL of 1×HEPES buffered saline was added and centrifuged until volume was decreased to 1 mL. Repeated once.
8. 1 mL of 1×HEPES buffered saline was added and centrifuged until volume was decreased to 0.5 mL.
9. The final sample was collected and filtered through 0.2 μm syringe filter with PES membrane, and stored in 2 mL Eppendorf tube in 4 C fridge.

TABLE 10
Preparation of DNA solution (LNP 6)

| Component | Vol (uL) |
|---|---|
| 1 mg/mL dsDNA stock (uL) | 50 |
| Buffer (20 mM malic acid, 30 mM NaCl, pH 3.0) | 950 |
| Total dsDNA solution | 1000 |

3. N/P Ratios of dsDNA LNP Formulations

N/P ratio is the molar ratio between amines (N) in ionizable lipid and the phosphate (P) in the nucleic acid backbone. By varying the N/P ratio and PEGylated lipid content, the surface charge of Lipid 24 LNPs could be varied from negative to neutral, as listed in Table 11.

TABLE 11
Characterizations of dsDNA LNP formulations of ionizable lipid Lipid 24

| | | N/P molar | PS | | ZP | EE |
|---|---|---|---|---|---|---|
| LNP | Components of LNP (molar ratio) | ratio | (nm) | PDI | (mV) | (%) |
| LNP 7 | Lipid 24:Chol:DSPC:DMG- | 4.81 | 86 | 0.04 | −15.9 | 59 |
| | PEG2000:Tri-GalNAc = | | | | | |
| | 49.22:38.37:9.9:2:0.5 | | | | | |
| LNP 8 | Lipid 24:Chol:DSPC:DMG- | 4.81 | 92 | 0.05 | −13.2 | 66 |
| | PEG2000:Tri-GalNAc = | | | | | |
| | 49.22:39.37:9.9:1.01:0.5 | | | | | |
| LNP 6 | Lipid 24:Chol:DSPC:DMG- | 6 | 91 | 0.04 | −0.29 | 65 |
| | PEG2000:Tri-GalNAc = | | | | | |
| | 49.22:39.37:9.9:1.01:0.5 | | | | | |

### Example 6. PKA Values of LNPS of Novel Lipids

### 1. Preparation of LNP

A lipid solution for an LNP was prepared according to Table 12 and the following:
1. The lipid solution was prepared in BSC hood to ensure clean environment.
2. Autoclaved 4 mL glass vials and RNAse/DNAse-free conical tubes were used for lipid solution and DNA solution preparation, respectively.
3. All buffer used in preparation was filtered through 0.2 μm membrane.
4. The lipid solutions were prepared by mixing individual lipid stock solutions which were filtered through 0.2 μm PTFE syringe filters.
5. The DNA solution was prepared by mixing the DNA stock solution with desired buffer which was filtered thorough filter units with 0.2 μm membrane.
6. All prepared solutions were used within 1 hour for LNP fabrications.

TABLE 12
Preparation of lipid solution

| | Conc. of | Vol of | |
|---|---|---|---|
| | lipid stock | lipid stock | Lipid |
| | solution in | solution | molar |
| Component | ethanol (mM) | (uL) | ratio |
| Ionizable lipid | 20 | 94.9 | 49.22 |
| Chol | 20 | 75.9 | 39.37 |
| DSPC | 20 | 19.1 | 9.9 |
| DMG-PEG2K | 2 | 29.1 | 1.51 |
| Ethanol | — | 180.9 | — |
| Total lipid solution | — | 400 | — |

Note:
N/P molar ratio = 4.81.

### 2. Preparation of LNP

eGFP Nanoplasmid LNP formulations was prepared according to Table 13 and the following:
1. 0.9 mL of eGFP Nanoplasmid solution and 0.3 mL of lipid solution were loaded in two 1 mL BD syringes, respectively.
2. The syringes were loaded on Ignite with the desired parameters.
3. A 15 mL Falcon tube was loaded in the sample position and run Ignite with flow rate of 20 mL/min and flow ratio of 3:1.
4. 0.85 mL of LNPs were collected and immediately diluted by adding 4.25 mL of 1×HEPES buffered saline (25 mM HEPES, 150 mM NaCl, pH7.4).
5. The diluted LNPs sample was transferred to a 50 mL Amicon tubes with 100,000 kDa MWCO filter membrane for buffer exchange and concentration.
6. The Amicon tube with LNP sample was centrifuged @ 500rcf until volume was decreased to 1 mL.
7. 1 mL of 1×HEPES buffered saline was added and centrifuged until volume was decreased to 1 mL. Repeated once.
8. 1 mL of 1×HEPES buffered saline was added and centrifuged until volume was decreased to 0.3 mL.
9. The final sample was collected and filtered through 0.2 μm syringe filter with PES membrane, and stored in 2 mL Eppendorf tube in 4 C fridge.

TABLE 13
Preparation of eGFP Nanoplasmid solution (LNP 9)

| Component | Vol (uL) |
|---|---|
| 1 mg/mL eGFP Nanoplasmid stock (uL) | 100 |
| Buffer (20 mM malic acid, 30 mM NaCl, pH 3.0) | 900 |
| Total eGFP Nanoplasmid solution | 1000 |

### 3. pKa Values of LNPs of Novel Lipids

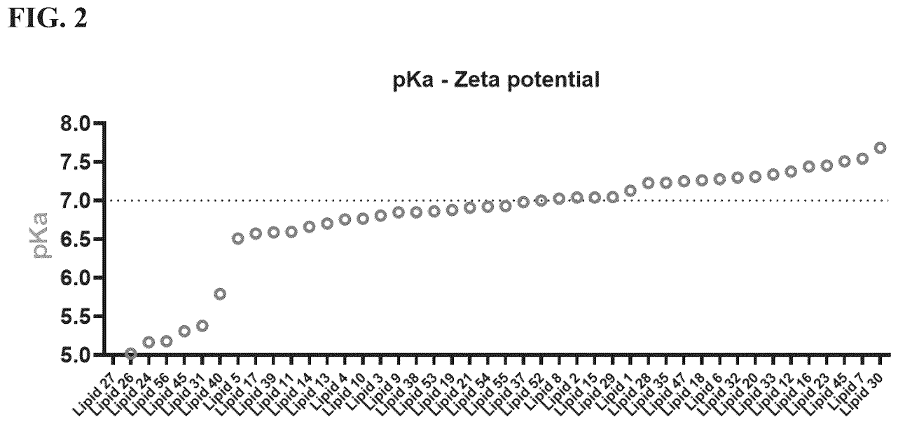

LNP's pKa values were measured using acid-base titration method with 2(p-toluidino)-6-naphthalene sulfonic acid (TNS) as the fluorescent marker. By design, the pKa values are distributed around 7.0 from pH 6.5 to pH7.6, with one exception of Lipid 24, whose pKa is very low to 5.167. pKa values of LNPs are provided in Table 14 and FIG. 2.

TABLE 14
pKa values of LNPs of novel lipids

| Formulation | Ionizable lipids | pKa (TNS) | R\hat{ }2 |
|---|---|---|---|
| LNP 43 | Lipid 27 | 4.82 | 0.99 |
| LNP 42 | Lipid 26 | 5.02 | 0.98 |
| LNP 10 | Lipid 24 | 5.17 | 1 |
| LNP 60 | Lipid 56 | 5.18 | 0.84 |
| LNP 53 | Lipid 45 | 5.31 | 1 |
| LNP 45 | Lipid 31 | 5.38 | 1 |
| LNP 52 | Lipid 40 | 5.79 | 0.97 |
| LNP 11 | Lipid 5 | 6.51 | 1 |
| LNP 9 | Lipid 17 | 6.57 | 1 |
| LNP 51 | Lipid 39 | 6.59 | 0.98 |
| LNP 12 | Lipid 11 | 6.6 | 0.99 |
| LNP 13 | Lipid 14 | 6.66 | 0.99 |
| LNP 14 | Lipid 13 | 6.7 | 0.99 |
| LNP 15 | Lipid 4 | 6.76 | 0.99 |
| LNP 16 | Lipid 10 | 6.77 | 1 |
| LNP 17 | Lipid 3 | 6.81 | 0.93 |
| LNP 18 | Lipid 9 | 6.85 | 0.99 |
| LNP 50 | Lipid 38 | 6.85 | 0.99 |
| LNP 57 | Lipid 53 | 6.86 | 0.99 |
| LNP 19 | Lipid 19 | 6.88 | 0.98 |
| LNP 20 | Lipid 21 | 6.91 | 0.99 |
| LNP 58 | Lipid 54 | 6.92 | 0.96 |
| LNP 59 | Lipid 55 | 6.93 | 0.99 |
| LNP 49 | Lipid 37 | 6.98 | 0.96 |
| LNP 56 | Lipid 52 | 7 | 0.98 |
| LNP 21 | Lipid 8 | 7.03 | 0.99 |
| LNP 23 | Lipid 15 | 7.04 | 0.99 |
| LNP 22 | Lipid 2 | 7.04 | 0.98 |
| LNP 44 | Lipid 29 | 7.05 | 0.91 |
| LNP 24 | Lipid 1 | 7.13 | 0.98 |
| LNP 25 | Lipid 28 | 7.23 | 0.97 |
| LNP 48 | Lipid 35 | 7.23 | 0.91 |
| LNP 55 | Lipid 47 | 7.25 | 0.8 |
| LNP 26 | Lipid 18 | 7.26 | 0.78 |
| LNP 27 | Lipid 6 | 7.28 | 0.99 |
| LNP 46 | Lipid 32 | 7.3 | 0.99 |
| LNP 28 | Lipid 20 | 7.31 | 0.97 |
| LNP 47 | Lipid 33 | 7.34 | 0.98 |
| LNP 29 | Lipid 12 | 7.38 | 0.98 |
| LNP 30 | Lipid 16 | 7.44 | 0.97 |
| LNP 31 | Lipid 23 | 7.45 | 0.98 |
| LNP 54 | Lipid 45 | 7.51 | 0.71 |
| LNP 32 | Lipid 7 | 7.54 | 0.95 |
| LNP 33 | Lipid 30 | 7.68 | 0.94 |

### Example 7. In Vivo Expression and Tissue Distribution of Fluc mRNA LNP Formulations

### 1. Lipid Solution for LNP Formulation Procedures

A lipid solution for an LNP was prepared according to Table 15 and following:
1. The lipid solution was prepared in BSC hood to ensure clean environment.
2. Autoclaved 4 mL glass vials and RNAse/DNAse-free conical tubes were used for lipid solution and DNA solution preparation, respectively.
3. All buffer used in preparation was filtered through 0.2 μm membrane.
4. The lipid solutions were prepared by mixing individual lipid stock solutions which were filtered through 0.2 μm PTFE syringe filters.
5. The mRNA solution was prepared by mixing the FLuc mRNA stock solution with desired buffer which was filtered thorough filter units with 0.2 μm membrane.
6. All prepared solutions were used within 1 hours for LNP fabrications.

TABLE 15
Preparation of lipid solution

| | Conc. of | Vol of | |
|---|---|---|---|
| | lipid stock | lipid stock | Lipid |
| | solution in | solution | molar |
| Component | ethanol (mM) | (uL) | ratio |
| Lipid 4 | 20 | 134.8 | 49.22 |
| Cholesterol | 45 | 47.9 | 39.38 |
| DSPC | 20 | 26.9 | 9.84 |
| DMG-PEG2K | 2 | 41.1 | 1.5 |
| DilC18(5)-DS | 0.5 | 6.6 | 0.06 |
| Ethanol | — | 222.7 | — |
| Total lipid solution | — | 480 | — |

Note:
N/P molar ratio = 6; DilC(18)-DS = 1,1-Dioctadecyl-3,3,3,3-tetramethylindodicarbocyanine-5,5-disulfonic acid.

2. Preparation of Fluc mRNA Solution

Fluc mRNA formulations was prepared according to Table 16.

TABLE 16
Preparation of FLuc mRNA solution

| Component | Vol (μL) |
|---|---|
| 1 mg/mL Fluc mRNA stock (uL) | 120 |
| Buffer (30 mM Sodium Acetate, 30 mM NaCl, pH 4.0) | 1080 |
| Total FLuc mRNA solution | 1200 |

3. Preparation of Fluc mRNA LNP Formulations

The lipid solution and the Fluc mRNA solution were used to prepare Fluc mRNA LNP, according to the following:
1. 1.1 mL of Fluc mRNA solution and 0.367 mL of lipid solution were loaded in a 3 mL BD syringe and a 1 mL BD syringe, respectively.
2. The syringes were loaded on Ignite with the desired parameters.
3. A 15 mL Falcon tube was loaded in the sample position and run Ignite.
1. 0.97 mL of LNPs were collected and immediately diluted by adding 3.87 mL of HEPES buffered saline (25 mM HEPES, 75 mM NaCl, 5% sucrose, pH7.4).
4. The diluted LNPs sample was transferred to a 50 mL Amicon tubes with 100,000 kDa MWCO filter membrane for buffer exchange and concentration.
5. The Amicon tube with LNP sample was centrifuged @ 500rcf until volume was decreased to 1 mL.
6. 1 mL of 1×HEPES buffered saline was added and centrifuged until volume was decreased to 1 mL. Repeated once.
7. 1 mL of 1×HEPES buffered saline was added and centrifuged until volume was decreased to 0.3 mL.
8. The final sample was collected and filtered through 0.2 μm syringe filter with PES membrane, and stored in 2 mL Eppendorf tube in 4 C fridge.

The obtained LNPs were characterized and demonstrated in Table 17.

TABLE 17
Characterizations of LNPs for in vivo screening

| | | N/P molar | PS | | ZP | EE | Endotoxin |
|---|---|---|---|---|---|---|---|
| LNP # | Lipids | ratio | (nm) | PdI | (mV) | (%) | (EU/mL) |
| LNP 34 | Lipid 4 | 6 | 75 | 0.04 | −7.2 | 92 | <0.2 |
| LNP 35 | Lipid C | 4.81 | 63 | 0.04 | −6.1 | 87 | <0.2 |
| LNP 36 | Lipid 6 | 4.81 | 72 | 0.05 | 0.1 | 85 | <0.2 |
| LNP 37 | Lipid 7 | 4.81 | 85 | 0.05 | 0.2 | 70 | <0.2 |
| LNP 38 | Lipid 12 | 4.81 | 75 | 0.02 | −0.4 | 88 | <0.2 |
| LNP 39 | Lipid 2 | 4.81 | 79 | 0.04 | −1.6 | 90 | <0.2 |
| LNP 40 | Lipid 1 | 4.81 | 81 | 0.07 | 1.8 | 82 | <0.2 |
| LNP 41 | Lipid 28 | 4.81 | 65 | 0.18 | 7.9 | 96 | <0.2 |

Note:
Lipid C is the control lipid of the structure below, used for comparison. See Cornebise et al., “Discovery of a Novel Amino Lipid That Improves Lipid Nanoparticle Performance through Specific Interactions with mRNA” Adv. Funct. Mater. 2022, 32, 2106727.

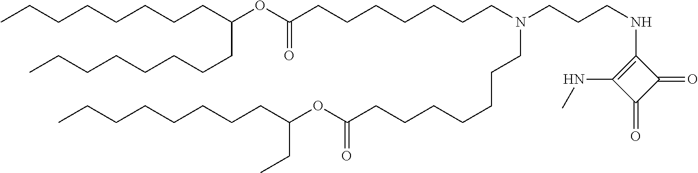 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

### 4. LNPs In Vitro Distribution and Expression

In vivo distribution and expression study was performed using the LNPs prepared in Table 17. Samples were dosed at 0.5 mg/kg to SKH1 female mice through tail IV injection. After 6 hours of injection, all mice were imaged for luciferase expression. After 24 hours, all mice were dissected and selected organs were removed and imaged for luciferase expression.

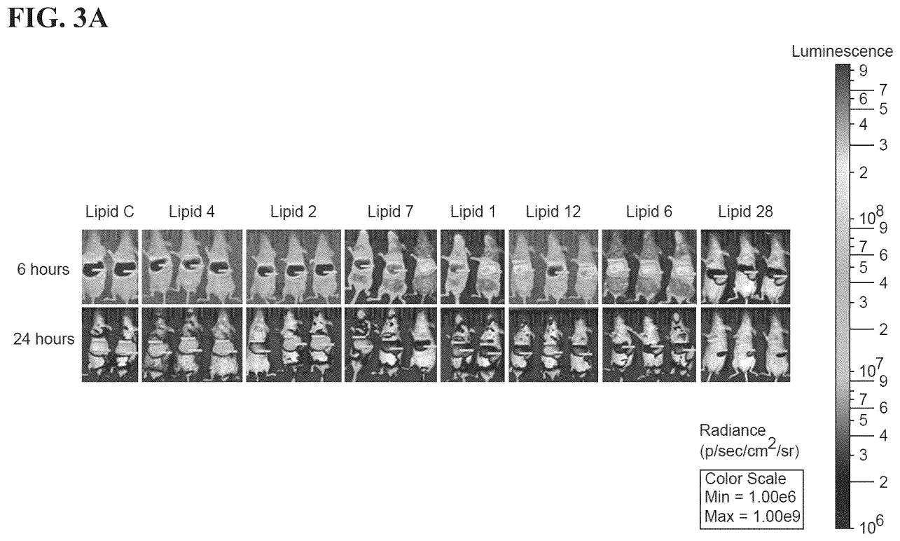

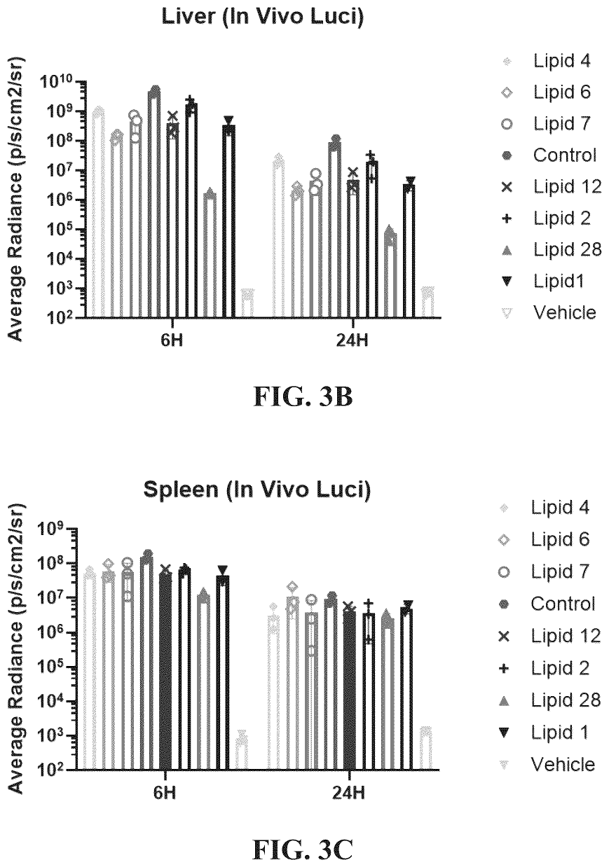

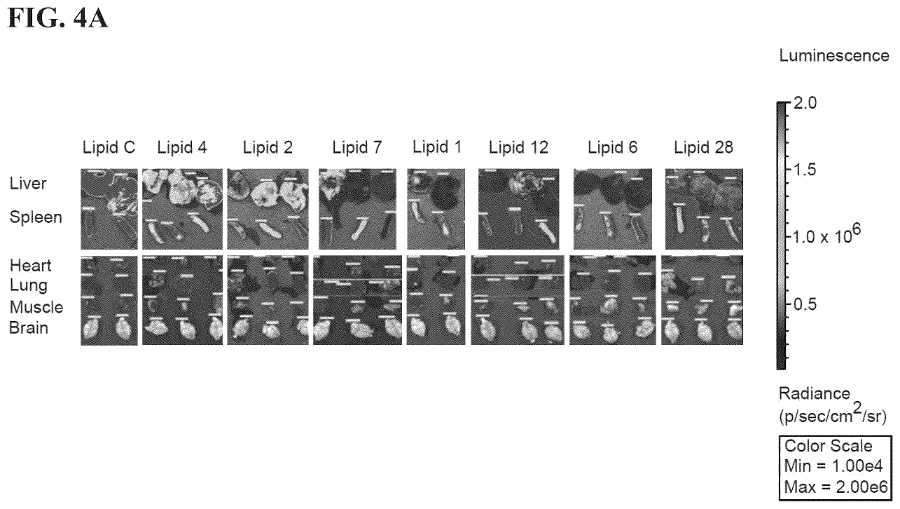

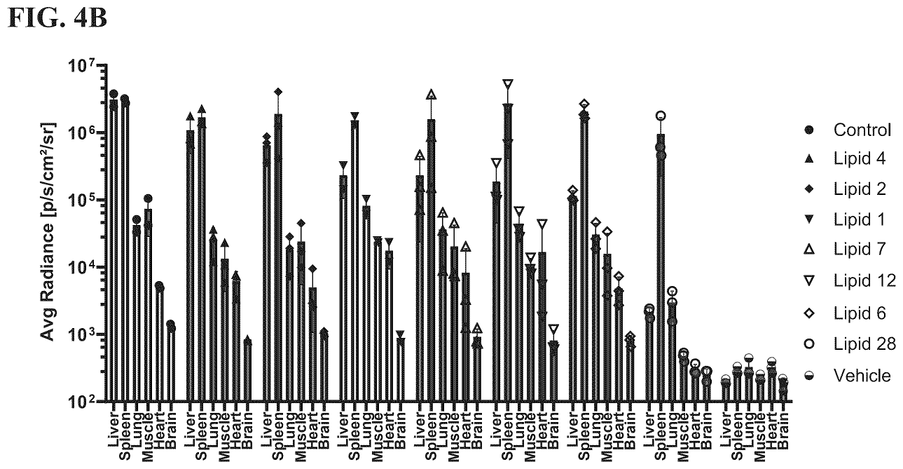

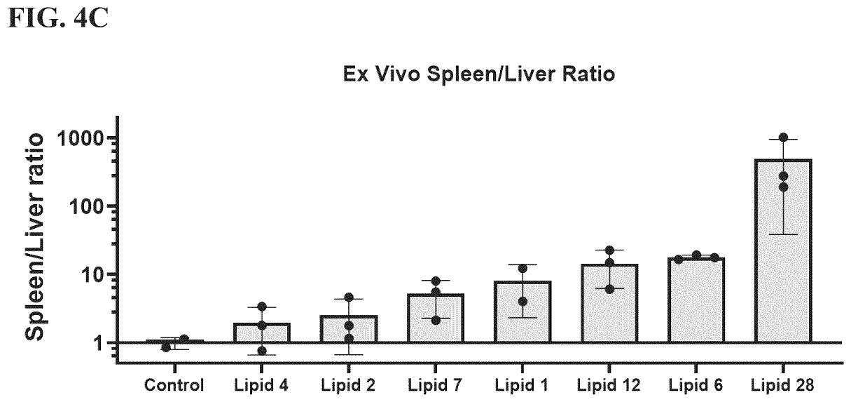

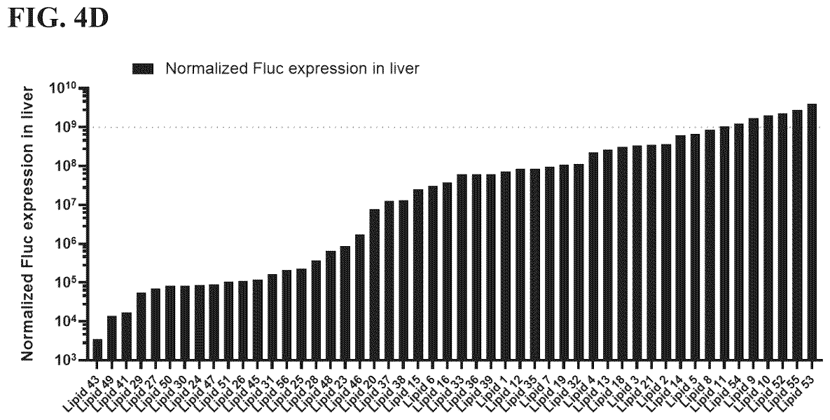

Compared to the control Lipid C, which targets liver, all the formulations of new lipids show a trend of decreased liver expression, while the spleen expressions are maintained at a similar level by in vivo imaging (FIG. 3A, FIG. 3B, and FIG. 3C). LNPs of lipid Lipid 28 gave the lowest liver expression. The ex vivo imaging for liver and spleen data (FIG. 4A) is consistent with the in vivo imaging results. By significantly de-targeting liver, the spleen/liver ratio of luciferase expression for LNPs of Lipid 28 reached to >500 by ex vivo imaging (FIG. 4A, FIG. 4B, and FIG. 4C). All spleen/liver ratios of other formulations range from 1 to 17.

The liver expressions of luciferase after 24 hours of LNP injection were plotted in FIG. 4D. It is possible to fine tune the tissue targeting by adjusting the lipid structures only. Here we demonstrate LNP formulation with strong spleen tropism was achieved successfully without using any target ligand or additional targeting excipient.

### Example 8: Transfection of Human Hematopoietic STEM Cells

The following example relates to enhanced transfection of human HSC in vivo using LNPs containing increased levels of helper lipids, such as neutral phospholipids, for example 20 mol % DSPC.

### Preparation and Characterization of Lipid Nanoparticles

Lipid nanoparticles (LNPs) encapsulating an mRNA cargo were prepared by mixing an aqueous mRNA solution and an ethanolic lipid blend solution (containing ionizable lipid, DSPC, DPG-PEG and Cholesterol at lipid ratios shown in TABLE 18) using an in-line microfluidic mixing process. The mRNA cargo was mCherry mRNA. Lipid nanoparticles were produced with different amounts of helper lipids, e.g., DSPC of 10 mol % or 20 mol %.

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were mounted into the luer ports of the mixing cartridge. The two solutions were then mixed at a 3:1 v/v ratio of mRNA solution (1.5 mL) to lipid solution (0.5 mL) at a total flow rate of 9 mL/min using the NanoAssemblr Ignite. The resulting suspension was held at room temperature for a minimum of 5 minutes before proceeding to ethanol removal and buffer exchange.

Following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with HBS exchange buffer (25 mM pH 7.4 HEPES buffer with 150 mM NaCl). The LNP suspension (2 mL) was then loaded into the device and centrifuged at 500 RCF until the volume was reduced by half volume (1 mL). The suspension was then diluted with exchange buffer (1 mL, 25 mM pH 7.4 HEPES buffer) to bring the suspension back to the original volume. This process of two-fold concentration and two-fold dilution was repeated five additional times for a total of six discontinuous diafiltration steps. The LNP suspension was then exchanged into MBS (25 mM pH 6.5 MES buffer with 150 mL NaCl) by diluting ten-fold with MBS and centrifuging at 500 RCF until the volume was reduced by one tenth. This ten-fold dilution with MBS and ten-fold concentration step was repeated one more time. The retentate containing the LNPs in MBS was recovered from the centrifugal ultrafiltration device and stored at 4° C. until further use.

Samples of the LNPs were characterized to determine the average hydrodynamic diameter, zeta potential, and mRNA content (total and dye-accessible mRNA). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer. The results for Formulation A and B LNPs are shown in TABLE 18.

TABLE 18

| | | | | | | DLS Z- | | Zeta | |
|---|---|---|---|---|---|---|---|---|---|
| | | | | | | Avg. | | Potential | |
| | | | | Fab | mRNA | Diameter | DLS | at pH 7.4 | Endotoxin |
| No. | Lipids | Lipid mol % | N/P | density | (μg/ml) | (nm) | PDI | (mV) | (EU/ml) |
| A | Lipid 1/Chol/ | 49.24/38.4/ | 4.81 | 12 | 150 | 80 | 0.1 | 1.4 | <0.4 |
| | DSPC/DPG- | 9.85/2.51 | | | | | | | |
| | PEG2K | | | | | | | | |
| B | Lipid 1/Chol/ | 49.24/28.25/ | 4.81 | 12 | 150 | 104 | 0.1 | 0.8 | <0.4 |
| | DSPC/DPG- | 20/2.51 | | | | | | | |
| | PEG2K | | | | | | | | |

### Preparation of Conjugates to Enable T Cell and Hematopoietic Stem Cell Targeting

Antibody fragments that bind to HSC-specific targets (CD117) were conjugated to DSPE-PEG (3.4 K)-maleimide and DSPE-PEG (2 k)-maleimide, respectively, via covalent coupling between the maleimide group and a C-terminal cysteine in the heavy chain (HC). In a representative reaction, the protein solution in PBS with S mM EDTA was concentrated to 3-4 mg/mL using a 10 kDa MWCO regenerated cellulose centrifugal filter. C-terminal cysteine residues were selectively reduced using 2-5 mM. TCEP at room temperature for 1.5 hours. TCEP was removed using a 7K MWCO spin desalting column. The reduced protein was mixed with a mixture of DSPE-PEG-Mal and DSPE-PEG-OMe at a 1:1 molar ratio of DSPE-PEG-Mal to Fab or V_{HH} and a 1:4 molar ratio of DSPE-PEG-Mal:DSPE-PEG-OMe. The reaction mixture was incubated at 37° C. for 2 hours and then quenched using 15 mM cysteine at 37° C. for 15 min. The resulting DSPE-PEG-Fab or DSPE-PEG-V_{HH} micelles were then concentrated and buffer-exchanged into pH 7.4 HEPES buffered saline using a 50K MWCO regenerated cellulose centrifugal filter. The concentration/buffer exchange process also removes residual cystine and unreacted Fab or VHH. The material was characterized by SDS-PAGE and HPLC. Fab or VHH concentration was determined by measuring the absorbance at 280 nm.

### Transfection of Human Hematopoietic Stem Cells

CD117+/CD34+ human hematopoietic stem cells (HSCs) were used to generate humanized NSG™ mice. HSC-targeted LNPs coated with anti-CD117 Fab were engineered using LNP formulations comprising either 10 mol % DSPC (Formulation A) or 20 mol % DSPC (Formulation B).

Humanized NSG™ mice were treated, by intravenous injection, with 1 mg/kg of LNP encapsulating mCherry mRNA coated with the anti-CD117 Fab and transfection efficiency was measured in NSG™ bone marrow.

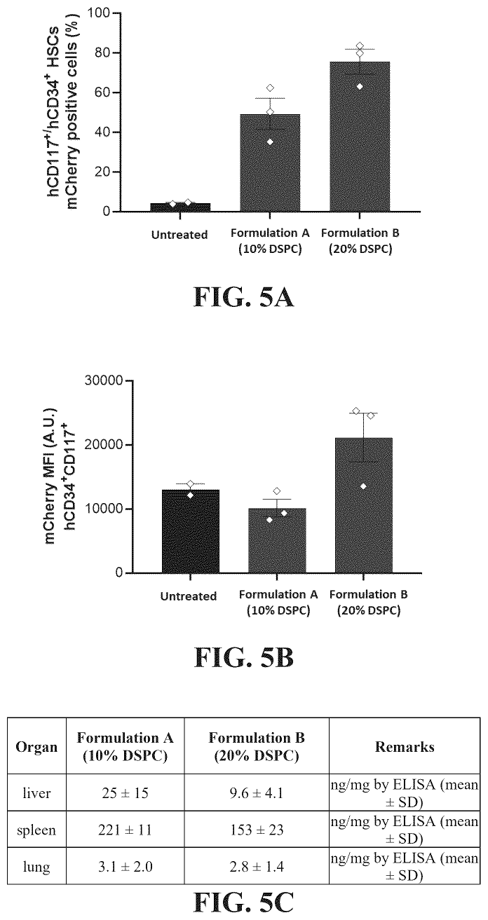

In vivo transfection efficiency increased from 50% for Formulation A to 75% for Formulation B (FIG. 5A). A 2-fold increase of median fluorescence intensity was measured in Formulation B transfected HSCs compared to Formulation A transfected HSCs in vivo. (FIG. 5B). Off-tissue targeting signal measured in the liver, spleen, and lung were lower in NSG™ mice treated with 20% mol % DPSC LNP Formulation B compared to 10 mol % DPSC LNP Formulation A (FIG. 5C).

### Example 9: Delivery of LNPS to Placenta for Luciferase Expression

### 1. Lipid Solution for LNP Formulation Procedures

A lipid solution for an LNP was prepared according to Table 19 and following:
1. The lipid solution was prepared in BSC hood to ensure clean environment.
2. Autoclaved 4 mL glass vials and RNAse/DNAse-free conical tubes were used for lipid solution and DNA solution preparation, respectively.
3. All buffer used in preparation was filtered through 0.2 μm membrane.
4. The lipid solutions were prepared by mixing individual lipid stock solutions which were filtered through 0.2 μm PTFE syringe filters.
5. The mRNA solution was prepared by mixing the FLuc mRNA stock solution with desired buffer which was filtered thorough filter units with 0.2 μm membrane.
6. All prepared solutions were used within 1 hours for LNP fabrications.

TABLE 19
Preparation of lipid solution

| | Conc. of | Vol of | |
|---|---|---|---|
| | lipid stock | lipid stock | Lipid |
| | solation in | solation | molar |
| Component | ethanol (mM) | (uL) | ratio |
| Lipid 1 | 20 | 225.1 | 49.22 |
| Cholesterol | 45 | 80.0 | 39.38 |
| DSPC | 20 | 45.0 | 9.84 |
| DMG-PEG2K | 2 | 6.9 | 1.5 |
| DilC18(5)-DS | 0.5 | 11.0 | 0.06 |
| Ethanol | — | 632.0 | — |
| Total lipid solution | — | 1000 | — |

Note:
N/P molar ratio = 6; DilC(18)-DS = 1,1-Dioctadecyl-3,3,3,3-tetramethylindodicarbocyanine-5,5-disulfonic acid.

2. Preparation of Fluc mRNA Solution

Fluc mRNA formulations was prepared according to Table 20.

TABLE 20
Preparation of FLuc mRNA solution

| Component | Vol (uL) |
|---|---|
| 1 mg/mL Fluc mRNA stock (uL) | 250 |
| Buffer (30 mM Sodium Acetate, 30 mM NaCl, pH 4.0) | 2250 |
| Total FLuc mRNA solution | 2500 |

3. Preparation of Fluc mRNA LNP Formulations

The lipid solution and the Fluc mRNA solution were used to prepare Fluc mRNA LNP, according to the following:
1. 2.4 mL of Fluc mRNA solution and 0.8 mL of lipid solution were loaded in a 3 mL BD syringe and a 1 mL BD syringe, respectively.
2. The syringes were loaded on Ignite with the desired parameters.
3. A 15 mL Falcon tube was loaded in the sample position and run Ignite.
4. 2.7 mL of LNPs were collected and immediately diluted by adding 10.8 mL of HEPES buffered saline (25 mM HEPES, 75 mM NaCl, 5% sucrose, pH7.4).
5. The diluted LNPs sample was transferred equally to two 50 mL Amicon tubes with 100K filter membrane for buffer exchange and concentration.
6. The Amicon tubes with LNP sample were centrifuged @ 500rcf until volume was decreased to 1 mL. The LNP samples in each Amicon tube was combined into one Amicon tube.
7. 2 mL of 1×HEPES buffered saline was added and centrifuged until volume was decreased to 2 mL. Repeated once, and the final volume was decreased to 1 mL.
8. 1 mL of 1×HEPES buffered saline was added and centrifuged until volume was decreased to 0.5 mL.
9. The final sample was collected and filtered through 0.2 μm syringe filter with PES membrane, and stored in 2 mL Eppendorf tube in 4 C fridge.

The obtained LNPs were characterized and demonstrated in Table 21.

TABLE 21
Characterizations of LNPs for in vivo screening

| | | N/P | | | | | |
|---|---|---|---|---|---|---|---|
| | | molar | PS | | ZP | EE | Endotoxin |
| Formulation | Lipids | ratio | (nm) | PdI | (mV) | (%) | (EU/mL) |
| LNP 61 | Lipid 1 | 4.81 | 115 | 0.05 | −0.3 | 66 | <0.4 |
| LNP 62 | Lipid 12 | 4.81 | 91 | 0.05 | 2.9 | 74 | <0.4 |
| LNP 63 | Lipid 35 | 4.81 | 96 | 0.06 | −0.1 | 82 | <0.4 |
| LNP 64 | Lipid 28 | 4.81 | 66 | 0.06 | 9.7 | 97 | <0.4 |
| LNP 65 | Lipid 45 | 4.81 | 69 | 0.02 | 7.4 | 78 | <0.4 |
| LNP 66 | Lipid 53 | 4.81 | 79 | 0.05 | −8.4 | 54 | <0.4 |

Note:
The lipid ratio of Lipid 53 LNPs is Lipid 53:Cholesterol:DSPC:DMG-PEG:DilC18(5)-DS = 49.22:29.28:19.94:1.5:0.06.

### 4. LNPs In Vivo Distribution and Expression in Pregnant Mice

In vivo distribution and expression study was performed using the LNPs prepared in Table 21. Samples were dosed at 0.5 mg/kg to C57B1/6 embryonic day 14 (E14) mice through tail IV injection. After 24 hours, IVIS images were taken following luciferin injection. Placenta and fetus were removed and imaged for luciferase expression.

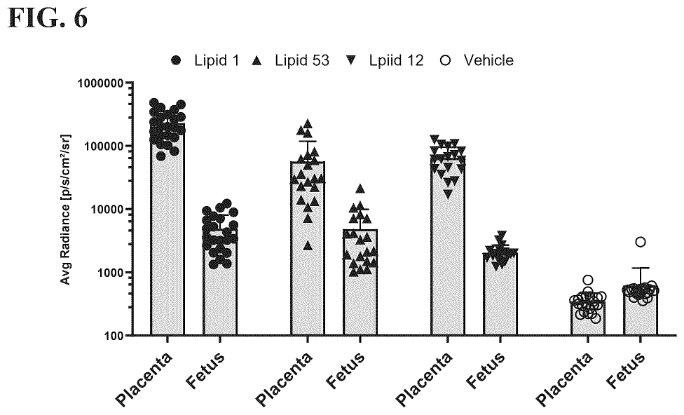

Three formulations (LNP 61 comprising Lipid 1; LNP 62 comprising Lipid 12; and LNP 66 comprising Lipid 53) demonstrate clear placenta expression, while two of them are fetus-free expression (FIG. 6).

### INCORPORATION BY REFERENCE

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, scientific articles, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1: A compound of Formula (I): 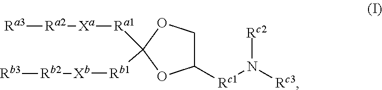
or a salt thereof, wherein:
R^{a1} and R^{b1} are each independently C₁₋₁₂ alkylene;
X^{a} and X^{b} are each independently —C(O)O—* or —OC(O)—*, wherein * indicates the point of attachment to R^{a1} or R^{b1},
R^{a2} and R^{b2} are each independently a bond or C₁₋₃ alkylene;
R^{a3} is 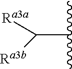
and R^{b3} is 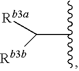
wherein R^{a3a}, R^{a3b}, R^{b3a}, and R^{b3b} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3;
R^{c1} is C₁₋₆ alkylene;
R^{c2} is H or C₁₋₆ alkyl; and
R^{c3} is C₁₋₆ alkyl, 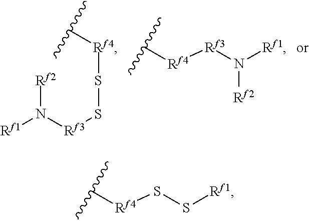
wherein:
R^{f1} is H, C₁₋₆ alkyl, or 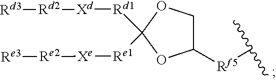
R^{f2} is H, C₁₋₆ alkyl, or —C(O)O—C₂₋₆ alkenyl;
R^{f3}, R^{f4}, and R^{f5} are each independently C₁₋₆ alkylene; and
R^{d1} and R^{e1} are each independently C₁₋₁₂ alkylene;
X^{d} and X^{e} are each independently —C(O)O—* or —OC(O)—*, wherein * indicates the point of attachment to R^{d1} or R^{c1};
R^{d2} and R^{e2} are each independently a bond or C₁₋₃ alkylene; and
R^{d3} is 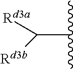
and R^{c3} is 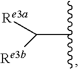
wherein R^{d3a}, R^{d3b}, R^{c3a}, and R^{c3b} are each independently H, C₁₋₁₂ alkyl optionally substituted with heterocylyl, or —(C₁₋₁₀ alkylene)-Sₙ—(C₁₋₁₀ alkyl), wherein n is each independently 1, 2, or 3;
with the proviso that when R^{c1} is —CH₂— and R^{c2} and R^{c3} are each methyl, then none of R^{a3a}, R^{a3b}, R^{b3a}, and R^{b3b} is H; when R^{c1} is —(CH₂)₂— and R^{c2} and R^{c3} are each methyl, then R^{b3a} is not H and R^{b3b} is ethyl; when R^{c1} is —(CH₂)₂— and R^{c2}, R^{c3}, or both R^{c2} and R^{c3} are not methyl, then none of R^{a3a}, R^{a3b}, R^{b3a}, and R^{b3b} is H; when R^{c1} is —(CH₂)₃—, R^{c2} and R^{c3} are each methyl, and one of R^{a3a} R^{a3b}, R^{b3a}, and R^{b3b} is H, then at least one of R^{a3a}, R^{a3b}, R^{b3a}, and R^{b3b} that is not H is substituted with a heterocylyl; and when R^{c1} is —(CH₂)₄—, R^{c2} and R^{c3} are each methyl, then none of R^{a3a}, R^{a3b}, R^{b3a}, and R^{b3b} is H.

2: The compound of claim 1, or a salt thereof, wherein R^{a1} and R^{b1} are each independently a linear C₁₋₁₂ alkylene; X^{a} and X^{b} are each —C(O)O—*, or X^{a} and X^{b} are each —OC(O)—*; and R^{a2} and R^{b2} are each a bond, or R^{a2} and R^{b2} are each —CH₂—.

3: The compound of claim 1, or a salt thereof, wherein R^{a3a}, R^{a3b} R^{b3a}, and R^{b3b} are each independently a linear C₁₋₁₂ alkyl.

4: The compound of claim 1, or a salt thereof, wherein at least one of R^{a3a}, R^{a3b}, R^{b3a}, and R^{b3b} is C₁₋₁₂ alkyl substituted with a 5- to 10-membered heterocyclyl comprising a disulfide bond or —(C₁₋₁₀ alkylene)-Sₙ—(C₁₋₁₀ alkyl).

5: The compound of claim 1, or a salt thereof, wherein R^{a3} and R^{b3} are each independently

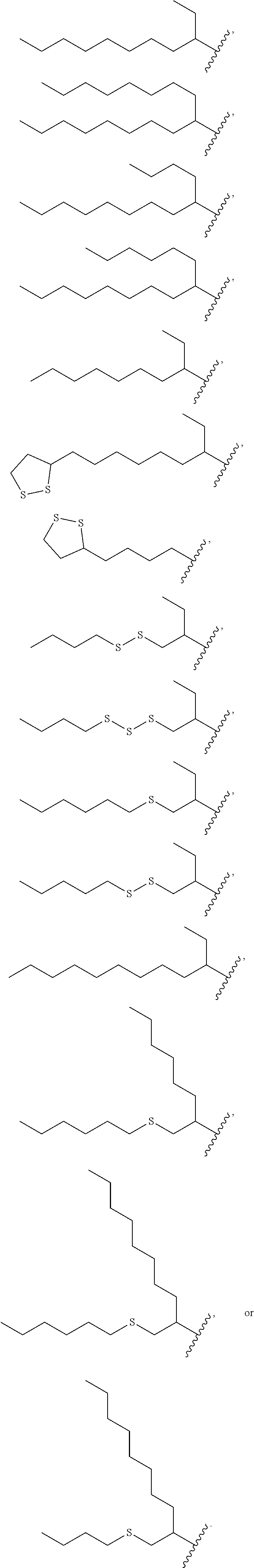

6: The compound of claim 1, or a salt thereof, wherein R^{c1} is —(CH₂)₂—, —(CH₂)₃—, or —(CH₂)₄—; and R^{c2} is methyl or ethyl.

7: The compound of claim 1, or a salt thereof, wherein R^{c3} is C₁₋₆ alkyl.

8: The compound of claim 1, or a salt thereof, wherein R^{c3} is methyl or ethyl, wherein when R^{c1} is —(CH₂)₂— and R^{c2} is methyl, then R^{c3} is not methyl.

9: The compound of claim 1, or a salt thereof, wherein R^{c3} is

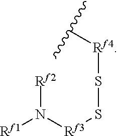

10: The compound of claim 1, or a salt thereof, wherein R^{c3} is

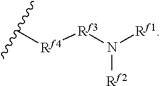

11: The compound of claim 1, or a salt thereof, wherein R^{c3} is

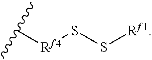

12: The compound of claim 9, or a salt thereof, wherein R^{f1} is C₁₋₆ alkyl.

13: The compound of claim 9, or a salt thereof, wherein R^{f1} is H, methyl, or n-butyl.

14: The compound of claim 9, or a salt thereof, wherein R^{f1} is

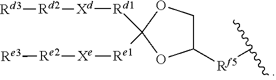

15: The compound of claim 9, or a salt thereof, wherein R^{f2} is H, methyl, ethyl, or —C(O)O—CH₂CH═CH₂; and R^{f3} and R^{f4} are each —(CH₂)₂—, or R^{f3} and R^{f4} are each —(CH₂)₃—.

16: The compound of claim 9, or a salt thereof, wherein R^{f4} is —(CH₂)₂—.

17: The compound of claim 14, or a salt thereof, wherein R^{f5} is —(CH₂)₂—, —(CH₂)₃—, or —(CH₂)₄—.

18: The compound of claim 14, or a salt thereof, wherein R^{d1} and R^{e1} are each independently a linear C₁₋₁₂ alkyelene; X^{d} and X^{e} are each —C(O)O—*, or X^{d} and X^{e} are each —OC(O)—*; R^{d2} and R^{e2} are each a bond, or R^{d2} and R^{e2} are each —CH₂—.

19: The compound of claim 14, or a salt thereof, wherein R^{d3a}, R^{d3b}, R^{c3a}, and R^{c3b} are each independently a linear C₁₋₁₂ alkyl.

20: The compound of claim 14, or a salt thereof, wherein at least one of R^{d3a}, R^{d3b}, R^{c3a}, and R^{c3b} is C₁₋₁₂ alkyl substituted with a 5- to 10-membered heterocyclyl comprising a disulfide bond or —(C₁₋₁₀ alkylene)-Sₙ—(C₁₋₁₀ alkyl).

21: The compound of claim 14, or a salt thereof, wherein R^{d3} and R^{e3} are each independently

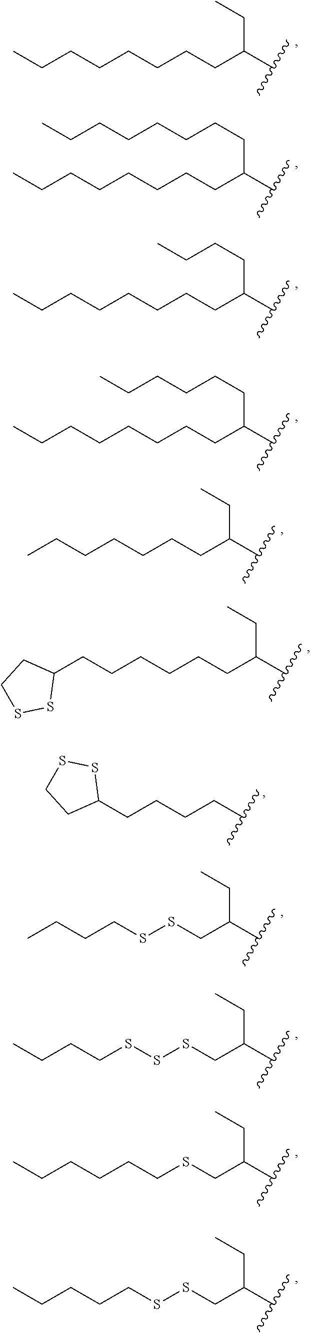

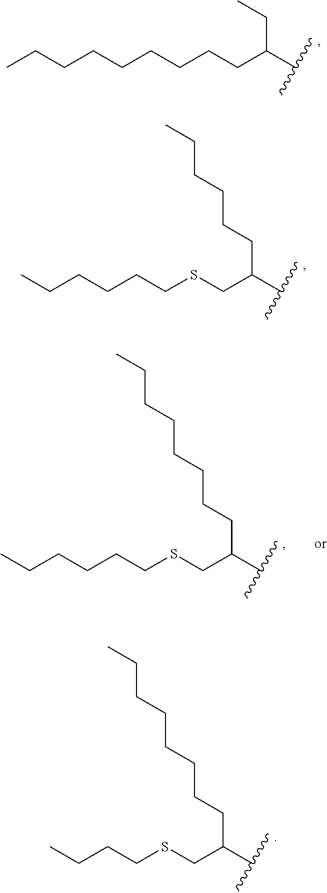 [SMILES: *C(CC)CCCCCCCCC ; *C(CCCCCC)CSCCCCCC ; *C(CCCCCCCC)CSCCCCCC ; *C(CCCCCCCC)CSCCCC]

22: The compound of claim 1, or a salt thereof, wherein the compound or the salt thereof is selected from the group consisting of the compounds of Table 1 and salts thereof.

23: A lipid nanoparticle (LNP) comprising a lipid blend for targeted delivery of a nucleic acid into an immune cell or a hematopoietic stem cell (HSC), wherein the lipid blend comprises an ionizable cationic lipid that is the compound of claim 1, or a salt thereof.

24: The LNP of claim 23, further comprising a lipid-cell targeting group conjugate comprising the compound of Formula (V): [Lipid]-[optional linker]-[cell targeting group], wherein the cell targeting group is an immune cell targeting group.

25: The LNP of claim 24, wherein the immune cell targeting group comprises an antibody that binds (i) a T cell antigen, wherein the T cell antigen is CD3, CD4, CD7, CD8, or any combination thereof (e.g., both CD3 and CD8, both CD4 and CD8, or both CD7 and CD8); (ii) a Natural Killer (NK) cell antigen, wherein the NK cell antigen is CD7, CD8, CD56, or any combination thereof (e.g., both CD7 and CD8); (iii) a macrophage antigen, a monocyte antigen, or a dendritic antigen, or any combination thereof, wherein the macrophage antigen comprises CDIIB, CD68, CD80, CD86, TRL-2, TRL-4, iNOS, MHC-II, CD163, CD206, CD209, FIZZ1, or Ym1/2, or a combination thereof; or any combination of (i) to (iii).

26: The LNP of claim 23, further comprising a lipid-cell targeting group conjugate comprising the compound of Formula (V): [Lipid]-[optional linker]-[cell targeting group], wherein the cell targeting group is an HSC targeting group.

27: The LNP of claim 26, wherein the HSC targeting group comprises an antibody that binds an antigen on the HSC comprising CD34, CD105, or CD117, or any combination thereof.

28: The LNP of claim 24, wherein the cell targeting group is covalently coupled to a lipid in the lipid blend via a polyethylene glycol (PEG) containing linker, wherein the lipid covalently coupled to the immune cell targeting group via a PEG containing linker is distearoylglycerol (DSG), distearoyl-phosphatidylethanolamine (DSPE), dimyristoyl-phosphatidylethanolamine (DMPE), distearoyl-glycero-phosphoglycerol (DSPG), dimyristoyl-glycerol (DMG), dipalmitoyl-phosphatidylethanolamine (DPPE), dipalmitoyl-glycerol (DPG), or ceramide.

29: The LNP of claim 23, wherein the lipid blend further comprises one or more of a structural lipid, a neutral phospholipid, and a free PEG-lipid.

30: The LNP of claim 29, wherein (i) the structural lipid is sterol; (ii) the neutral phospholipid is selected from the group consisting of phosphatidylcholine, phosphatidylethanolamine, distearoyl-sn-glycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin; and (iii) the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols, for example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyristoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]-N,N-ditetradecylacetamide, or a PEG-distearoyl-phosphatidylethanolamine (PEG-DSPE) lipid.

31: The LNP of claim 29, wherein the neutral phospholipid is 1,2-distearoyl-sn-glycero-3-phosphocholine (DSPC), wherein the neutral phospholipid is present in the lipid blend in a range of about 19 mol % to about 21 mol %.

32: The LNP of claim 23, further comprising a nucleic acid, wherein the nucleic acid is encapsulated in the LNP.

33: The LNP of claim 24, wherein the cell targeting group comprises an antibody, and the antibody is a Fab or an immunoglobulin single variable (ISV) domain (e.g., a Nanobody).

34: A method of targeting the delivery of a nucleic acid to a cell, the method comprising contacting the cell with the LNP of claim 23, wherein the LNP comprises the nucleic acid.

35: A method of treating, ameliorating, or preventing a symptom of a disorder or disease in a subject in need thereof, the method comprising administering to the subject an LNP of claim 23, wherein the LNP comprises a nucleic acid and delivers the nucleic acid into a cell of the subject.

36: A method of targeting the delivery of a nucleic acid to a non-liver cell, the method comprising contacting the non-liver cell with an LNP comprising the compound of claim 1, or a salt thereof, wherein (i) R^{x3} is 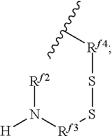
or (ii) R^{a3} and R^{b3} are each independently 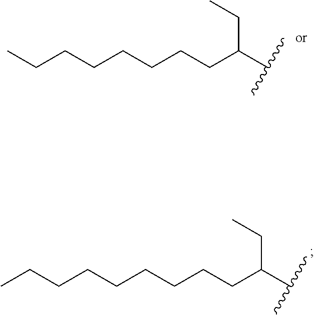 [SMILES: *C(CC)CCCCCCCC ; *C(CC)CCCCCCCCC]
or both (i) and (ii).

37: A method of targeting the delivery of a nucleic acid to a liver cell, the method comprising contacting the liver cell with an LNP comprising the compound of claim 1, or a salt thereof, wherein (i) R^{a3} and R^{b3} are each independently 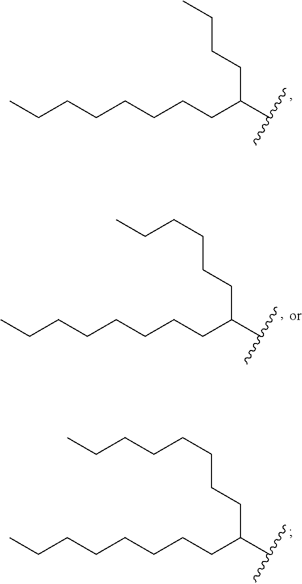 [SMILES: *C(CCCC)CCCCCCCC ; *C(CCCCCC)CCCCCCCC ; *C(CCCCCCCC)CCCCCCCC]
or at least one of R^{a3} and R^{b3} is

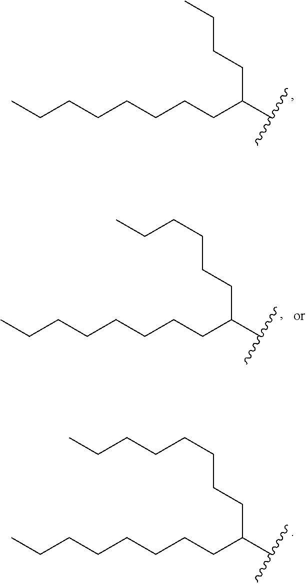 [SMILES: *C(CCCC)CCCCCCCC ; *C(CCCCCC)CCCCCCCC ; *C(CCCCCCCC)CCCCCCCC]

38: A method of targeting the delivery of a nucleic acid to a placental cell, the method comprising contacting the placental cell with an LNP comprising the compound of claim 1, or a salt thereof.
